(19) United States
(12) Patent Application Publication
(10) Pub. No.: US 2025/0297208 A1
(43) Pub. Date: Sep. 25, 2025

(54) YEAST WITH ENHANCED ASTAXANTHIN

(71) Applicant: Bioproton Pty Ltd, Brisbane (AU)

(72) Inventor: Carlos Luna-Flores, Brisbane (AU)

(21) Appl. No.: 18/848,173

(22) PCT Filed: Mar. 23, 2022
(86) PCT No.: PCT/AU2022/050258

§ 371 Date: Sep. 18, 2024

(51) Int. Cl. C12N 1/16; C12C 1/18; C12N 15/81; C12P 7/22; C12P 19/02; C12P 19/12

(52) U.S. Cl. CPC C12N 1/16; C12C 1/18; C12N 15/815; C12P 7/22; C12P 19/02; C12P 19/12; C12N 2840/102

(57) ABSTRACT

An isolated nucleic acid associated with increased accumulation of astaxanthin derived from a Xanthophyllomyces dendrorhous strain. The isolated nucleic acid comprising nucleotide sequence set forth in SEQ ID NOs: 1240-12684 or a nucleotide sequence at least 80% identical thereto, or a fragment of the isolated nucleic acid, is provided, wherein the nucleotide sequence of the isolated nucleic acid is not identical to any one of the nucleotide sequences set forth in SEQ ID NOs: 12685-19331. Associated proteins, genetic construct, cells, and methods are also provided.

### CROSS REFERENCE TO RELATED APPLICATIONS

This application is a U.S. National Stage under 35 U.S.C. § 371 of PCT/AU2022/050258 filed Mar. 23, 2022.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created May 16, 2025, is named “2025-05-16 Substitute Sequence Listing BPRT0001PA” and is 56,342,754 bytes in size.

### TECHNICAL FIELD

This invention relates to production of astaxanthin (AX). More particularly, the invention relates to biological production of AX such as using modified strains of Xanthophyllomyces dendrorhous.

### BACKGROUND

Astaxanthin (AX) is a carotenoid of the xanthophyll group known for its red-pinkish pigmentation. Due to its strong antioxidant properties, AX has significant application in health supplement and pharmaceutical industries. AX is also associated with desirable flesh colour in certain marine animals consumed as seafood such as shrimp, krill, crayfish, salmon, and trout. Generally, such animals cannot synthesise AX de-novo, and AX supplementation is used during production by aquaculture.

Currently, AX is synthesised primarily using petrochemical feedstock by a double Wittig reaction. AX can also be biologically synthesised, for example using the microalgae Haematococcus pluvialis, the gram-negative bacteria Paracoccus carotinifaciens, and the yeast Xanthophyllomyces dendrorhous (alternatively known as Phaffia rhodozyma). At present, the market size for pure AX is about 670 metric tonnes per annum, valued at about US$1.1 billion, with this market expected to exceed sales of US$2.25 billion by 2025. Synthetically produced AX is priced from about US$1,000 per kilogram, while biologically produced AX is priced from about US$7,000 per kilogram. Synthetic AX dominates the current global market due to its significantly lower cost, although health and safety concerns, environmental issues associated with the synthetic production process, and observed higher antioxidant activity of biologically produced AX as compared to synthetic AX, along with the trends towards use of natural products, sees significant current interest in the biological production of AX.

X. dendrorhous is a basidiomycetous yeast that produces AX as its main fermentation product using the mevalonate pathway. This yeast is typically preferred to other microbial AX producers for industrial purposes in view of superior growth rate, productivity, and robustness, and an ability to assimilate a wide diversity of carbon sources from feedstock or waste products including sucrose, glucose, fructose, xylose, glycerol, molasses, and bagasse hydrolysate (among others). Nevertheless, wild-type strains of X. dendrorhous produce relatively low yields of AX (200-400 μg/gDCW) which limits industrial efficacy. A detailed evaluation has suggested that AX yield, biomass density, and fermenter volume parameters of above 4,000 μg/gDCW, 60 g/L, and 1,500 L, respectively, could result in biological production of AX from X. dendrorhous that would be industrially competitive with existing synthetic approaches.

With the preceding in mind, new approaches for biological production of AX would be desirable. It would be particularly desirable, in at least some instances, to develop new approaches suitable for production of AX from yeast, particularly X. dendrorhous.

Reference to prior art in the background is not, and should not be taken to be, a suggestion that the prior art forms part of the common general knowledge in any jurisdiction.

### SUMMARY

A first aspect of the invention provides an isolated nucleic acid comprising a nucleotide sequence set forth in SEQ ID NOs:1240-12684 or a nucleotide sequence at least 80% identical thereto, or a fragment of the isolated nucleic acid.

Suitably, the nucleotide sequence of the isolated nucleic acid is not identical to any one of the nucleotide sequences set forth in SEQ ID NOs: 12685-19331.

In embodiments, the nucleotide sequence of the isolated nucleic acid is not identical to any one of the nucleotide sequences set forth in SEQ ID NOs:1-1239.

In embodiments, the nucleotide sequence of the isolated nucleic acid is or comprises a variant of a nucleotide sequence set forth in SEQ ID NOs:12685-19331. In embodiments, the nucleotide sequence is or comprises a variant of a nucleotide sequence set forth in SEQ ID NOs:1-1239.

The nucleotide sequence of the isolated nucleic acid may be a variant of a genic, regulatory, or intergenic region sequence as set out in Table 12. In embodiments, the variant nucleotide sequence is associated with a change in an amino acid sequence encoded by and/or expression of one or more CDS sequences set out in Table 12.

The nucleotide sequence of the isolated nucleic acid may be a variant of a genic, regulatory, or intergenic region sequence as set out in Table 13. In embodiments, the variant nucleotide sequence is associated with a change in amino acid sequence encoded by and/or expression of one or more CDS sequences set out in Table 13.

The nucleotide sequence of the isolated nucleic acid may be a variant of a genic, regulatory, or intergenic sequence as set out in Table 14. In embodiments, the variant is associated with a change in amino acid sequence encoded by and/or expression of one or more CDS sequences set out in Table 14.

The nucleotide sequence of the isolated nucleic acid may be a variant of a genic, regulatory, or intergenic sequence as set out in Table 15. In embodiments, the variant is associated with a change in amino acid sequence encoded by and/or expression of one or more CDS sequences set out in Table 15.

In embodiments, the nucleotide sequence of the isolated nucleic acid is a variant of a sequence encoding a transcript set out in Table 16.

In embodiments, the nucleotide sequence of the isolated nucleic acid is a variant of a regulatory sequence for a transcript as set out in Table 16.

In embodiments, the nucleotide sequence of the isolated nucleic acid is a variant of a sequence encoding a transcript set out in Table 17.

In embodiments, the nucleotide sequence of the isolated nucleic acid is a variant of a regulatory sequence for a transcript as set out in Table 17.

In embodiments, the nucleotide sequence of the isolated nucleic acid is a variant of a CDS sequence set forth in Table 18.

In embodiments, the nucleotide sequence of the isolated nucleic acid is a variant of a regulatory sequence for a CDS sequence set forth in Table 18.

In embodiments, the nucleotide sequence of the isolated nucleic acid is a variant of a CDS sequence set forth in Table 19.

In embodiments, the nucleotide sequence of the isolated nucleic acid is a variant of a regulatory sequence for a CDS sequence set forth in Table 19.

In embodiments, the nucleotide sequence of the isolated nucleic acid comprises a nucleotide change at a position of a variation X₂₂₃-X₈₃₉₅, as set out in Table 12. In embodiments, the nucleotide change is at a different position than any of the variations X₁-X₂₂₂, as set out in Table 12.

In embodiments, the nucleotide sequence of the isolated nucleic acid comprises a nucleotide change of a variation X₂₂₃-X₈₃₉₅, as set out in Table 12. In embodiments, the nucleotide change is different than any of the variations X₁-X₂₂₂, as set out in Table 12.

In embodiments, the nucleotide sequence of the isolated nucleic acid comprises a nucleotide change at a position of a variation Y₁-Y₃₆₈, as set out in Table 13. In embodiments, the nucleotide sequence comprises a nucleotide change of a variation Y₁-Y₃₆₈, as set out in Table 13.

In embodiments, the nucleotide sequence of the isolated nucleic acid comprises a nucleotide change at a position of a variation Z₁-Z₂₅, as set out in Table 14. In embodiments, the nucleotide sequence comprises a nucleotide change of a variation Z₁-Z₂₅, as set out in Table 14.

In embodiments, the nucleotide sequence of the isolated nucleic acid comprises a nucleotide change at a position of a V₁-V₂₆, as set out in Table 15. In embodiments, the nucleotide sequence comprises a nucleotide change of a variation V₁-V₂₆, as set out in Table 15.

In embodiments, the isolated nucleic acid comprises a nucleotide sequence set forth in SEQ ID NOs:1240-2795 or a variant thereof, wherein the nucleotide sequence or variant thereof is not identical to any one of the nucleotide sequences set forth in SEQ ID NOs:12685-19331. In embodiments, the nucleotide sequence or variant thereof is not identical to any one of the nucleotide sequences set forth in SEQ ID NOs:1-1239.

In embodiments, the isolated nucleic acid comprises a nucleotide sequence set forth in SEQ ID NOs:2796-4360 or a variant thereof, wherein the nucleotide sequence or variant thereof is not identical to any one of the nucleotide sequences set forth in SEQ ID NOs:12685-19331. In embodiments, the nucleotide sequence or variant thereof is not identical to any one of the nucleotide sequences set forth in SEQ ID NOs:1-1239.

In embodiments, the isolated nucleic acid comprises a nucleotide sequence set forth in SEQ ID NOs:4361-5500 or a variant thereof, wherein the nucleotide sequence or variant thereof is not identical to any one of the nucleotide sequences set forth in SEQ ID NOs:12685-19331. In embodiments, the nucleotide sequence or variant thereof is not identical to any one of the nucleotide sequences set forth in SEQ ID NOs:1-1239.

In embodiments, the isolated nucleic acid comprises a nucleotide sequence set forth in SEQ ID NOs:5501-6921 or a variant thereof, wherein the nucleotide sequence or variant thereof is not identical to any one of the nucleotide sequences set forth in SEQ ID NOs:12685-19331. In embodiments, the nucleotide sequence or variant thereof is not identical to any one of the nucleotide sequences set forth in SEQ ID NOs:1-1239.

In embodiments, the isolated nucleic acid comprises a nucleotide sequence set forth in SEQ ID NOs:6922-8057 or a variant thereof, wherein the nucleotide sequence or variant thereof is not identical to any one of the nucleotide sequences set forth in SEQ ID NOs:12685-19331. In embodiments, the nucleotide sequence or variant thereof is not identical to any one of the nucleotide sequences set forth in SEQ ID NOs:1-1239.

In embodiments, the isolated nucleic acid comprises a nucleotide sequence set forth in SEQ ID NOs:8058-9311 or a variant thereof, wherein the nucleotide sequence or variant thereof is not identical to any one of the nucleotide sequences set forth in SEQ ID NOs:12685-19331. In embodiments, the nucleotide sequence or variant thereof is not identical to any one of the nucleotide sequences set forth in SEQ ID NOs:1-1239.

In embodiments, the isolated nucleic acid comprises a nucleotide sequence set forth in SEQ ID NOs:9312-11033 or a variant thereof, wherein the nucleotide sequence or variant thereof is not identical to any one of the nucleotide sequences set forth in SEQ ID NOs:12685-19331. In embodiments, the nucleotide sequence or variant thereof is not identical to any one of the nucleotide sequences set forth in SEQ ID NOs:1-1239.

In embodiments, the isolated nucleic acid comprises a nucleotide sequence set forth in SEQ ID NOs:11034-12684 or a variant thereof, wherein the nucleotide sequence or variant thereof is not identical to any one of the nucleotide sequences set forth in SEQ ID NOs:12685-19331. In embodiments, the nucleotide sequence or variant thereof is not identical to any one of the nucleotide sequences set forth in SEQ ID NOs:1-1239.

A second aspect of the invention provides an isolated protein encoded by an isolated nucleic acid comprising a nucleotide sequence set forth in SEQ ID NOs:1240-12684 or a nucleotide sequence at least 80% identical thereto, or a fragment or derivative of the isolated protein. Suitably, the isolated protein of the second aspect is encoded by the isolated nucleic acid of the first aspect.

In embodiments, the isolated protein of the first aspect is of a protein classification selected from: short-chain dehydrogenase; deoxyribodipyrimidine photolyase/cryptochrome; nuclear transport factor 2; 60s ribosomal protein 132; armadillo/beta-catenin-like repeat-containing protein; 60s ribosomal protein 110a; pyridoxamine 5′-phosphate oxidase-like, FMN-binding domain; glutaredoxin-related protein; glycosyl transferase, family 8-glycogenin; mitochondrial carrier; nucleosome assembly protein; sterile alpha motif, type 2; snare protein ykt6; UDP-glucose dehydrogenase; predicted translation factor, contains W2 domain; G-protein beta subunit-like protein; heat shock protein HSS1; 40s ribosomal protein s7; ATP synthase f1 beta subunit; catalase 1; stress responsive alpha-beta barrel; cytokinin riboside 5′-monophosphate phosphoribohydrolase LOG; EF-hand domain pair; 20s proteasome subunit; ferrochelatase; glycine hydroxymethyltransferase; carboxypeptidase s; NADH-ubiquinone oxidoreductase 304 kDa subunit precursor; phytoene dehydrogenase; ribosomal protein L49/IMG2; nop10p-domain-containing protein; thioredoxin/protein disulfide isomerase; predicted dehydrogenase; 6-phosphogluconate dehydrogenase; NADH-dehydrogenase (ubiquinone); COPII vesicle protein; ornithine aminotransferase; ER-associated protein catabolism-related protein; isocitrate dehydrogenase; AAA atpase; probable NADP-dependent dehydrogenase acting on 3-hydroxy acids; CNDP dipeptidase; actin-related protein Arp2/3 complex, subunit ARPC2; branched-chain amino acid aminotransferase ii; carbon-nitrogen hydrolase; aspartate aminotransferase; NADPH oxidase; 26s proteasome subunit p45; pre-mRNA-splicing factor rsel; porphobilinogen deaminase; prolyl oligopeptidase; ABC transporter; 40s ribosomal protein s9; polyadenylate-binding protein; ATP-dependent RNA helicase dhx8; fatty acid synthase complex subunit alpha; glycosyltransferase family 35 protein; WD repeat protein; heat shock protein 60; succinate dehydrogenase; translocase of outer mitochondrial membrane complex, subunit TOM70/TOM72; nucleic acid-binding protein; nucleotide excision repair factor NEF2, RAD23 component; t-complex protein alpha subunit (tcp-1-alpha); k506-binding protein 2; aromatic amino acid aminotransferase; adenylate kinase; alpha-aminoadipate reductase lys1p; coatomer protein subunit alpha; 40s ribosomal protein s21; carbamoyl-phosphate synth; histone acetyltransferase SAGA, TRRAP/TRA1 component, PI-3 kinase superfamily; SAM-dependent RNA methyltransferase; related to 2-hydroxy-3-oxopropionate reductase; transcriptional coactivator p100; 60s ribosomal protein 113a; ornithine carbamoyltransferase; eukaryotic translation initiation factor 5b; aconitate hydratase; RNA 2-o-methyltransferase fibrillarin; t-complex protein beta subunit (tcp-1-beta); voltage-dependent ion-selective channel; coatomer beta subunit; succinate-ligase (adp-forming); carbamoyl-phosphate synthase; related to ste23-metalloprotease involved in a-factor processing; microtubule binding protein; pyridoxalphosphate-dependent enzyme/predicted threonine synthase; fact complex subunit SPT16; SLY1 vesicle trafficking secl-like protein; cytoplasm protein; NADH dehydrogenase; phosphoglycerate kinase; arm repeat-containing protein; ribonuclease III domain; GTP binding protein 4; peptidyl-prolyl cis-trans isomerase b; Translation initiation factor 4F, ribosome/mRNA-bridging subunit (eIF-4G); eukaryotic polypeptide chain release factor 3; asparagine synthase (glutamine-hydrolyzing); splicing factor U2AF, large subunit (RRM superfamily); NADH-cytochrome b5 reductase; histidine biosynthesis trifunctional-protein; Enoyl-CoA hydratase; alcohol; imidazoleglycerol phosphate synthase; thioredoxin-like fold; ef-hand; electron-transferring-flavoprotein dehydrogenase; MDF1-domain-containing protein; transcription factor IIS, N-terminal; heat shock protein 70; pyruvate carboxylase; homoaconitate hydratase; uncharacterized conserved coiled-coil protein; alternative splicing factor SRp55/B52/SRp75 (RRM superfamily); eukaryotic translation initiation factor 3 subunit 7; threonyl-trna synthetase; RmlC-like jelly roll fold; 60s ribosomal protein 120; mRNA splicing factor; pre-mrna-processing protein 45; atp-dependent rma helicase rrp3; dihydrolipoyllysine-residue acetyltransferase; Acyl-CoA synthetase; ribosomal protein S5; phenylalanyl-tRNA synthetase subunit beta; wd40 repeat-like protein; vacuolar ATP synthase subunit d; phosphatidylserine decarboxylase; vigilin; RNA recognition motif domain; plasma membrane h( )-atpase 1; RRM motif-containing protein; predicted GTPase-activating protein; F1-ATP synthase assembly protein; acetyl-hydrolase; peptidyl-prolyl cis-trans isomerase; antiviral helicase; acetyl CoA carboxylase; age pka protein kinase; ATP-dependent RNA helicase pitchoune; Microtubule-associated protein; cell-cycle nuclear protein, contains WD-40 repeats; phosphoserine aminotransferase; vacuolar protein sorting-associated protein; GMP synthase; translational regulator gcn20-like abc transporter; GDP-mannose pyrophosphorylase; acetyl CoA acyltransferase 2; phosphoketolase; delta 12 fatty acid desaturase; vacuolar protein 8; predicted haloacid-halidohydrolase and related hydrolases; class iii adh enzyme; t-complex protein 1; isocitrate lyase; atpase; 6-phosphogluconolactonase; mitochondrial inner membrane protein; t-complex protein 1 subunit delta; adaptor protein complex ap-1 gamma subunit; rRNA processing protein Rrp5; succinate:fumarate antiporter; predicted proline-serine-threonine phosphatase-interacting protein (PSTPIP); phospho-2-dehydro-3-deoxyheptonate aldolase; RNA-binding domain-containing protein; epsilon DNA polymerase; cullins; asparaginyl-tRNA synthetase; dihydroxy-acid dehydratase; SNARE protein SED5/Syntaxin 5; centromere microtubule binding protein cbf5; histidyl-trna synthetase; endoplasmic reticulum protein EP58, contains filamin rod domain and KDEL motif; 3-isopropylmalate dehydrogenase; Glycosyl transferase, family 1; eukaryotic translation initiation factor 3 subunit 6; phosphoglycerate mutase family; chromatin remodelling complex ATPase chain; predicted hydrolases or acyltransferases (alpha/beta hydrolase superfamily); NADH dehydrogenase subunits 2, 5, and related proteins; synaptobrevin-like protein; 40s ribosomal protein s6; ubiquitin C-terminal hydrolase UCHL 1; polyC-binding proteins alphaCP-1 and related KH domain proteins; nucleolar RNA-associated protein (NRAP); WD40 repeat-containing protein; pyruvate decarboxylase; RhoGEF GTPase; Ca2-dependent lipid-binding protein CLB1/vesicle protein vp115/Granuphilin A, contains C2 domain; molecular co-chaperone STI1; vacuolar H-ATPase V1 sector, subunit E; p-loop containing nucleoside triphosphate hydrolase protein; spliceosome subunit; microtubule-binding protein involved in cell cycle control; karyopherin (importin) beta 3; DNA-dependent RNA polymerase ii second largest subunit; coatomer subunit gamma; dehydrogenase kinase; mitochondrial pyruvate dehydrogenase el component beta subunit; glycoside hydrolase family 13 protein; NAD-specific glutamate dehydrogenase; mitochondrial 50s ribosomal protein 13; Ran GTPase-activating protein; FKBP-type peptidyl-prolyl cis-trans isomerase; 60s ribosomal protein 119; small nuclear ribonucleoprotein splicing factor; mannosyltransferase; dUTP pyrophosphatase; GST, gst; glutamate-trna ligase; mov34-domain-containing protein; mitochondrial nuclease; 1,4-benzoquinone reductase-like; thiamine biosynthetic bifunctional enzyme; protein of unknown function DUF3602; upf0041-domain-containing protein; 60s ribosomal protein 111; serine/threonine protein phosphatase 2A, regulatory subunit; argininosuccinate lyase; elongation factor 1 beta delta chain; bar-domain-containing protein; uridylate kinase; phosphatidylethanolamine n-methyltransferase; stomatin family protein; ubiquitin-conjugating enzyme; glycosyltransferase family 2 protein; signal recognition particle protein; B-cell receptor-associated protein and related proteins; RNA-binding S4 domain; Drebrins and related actin binding proteins; small gtpase-binding protein; gtp cyclohydrolase i; ps16 protein; predicted hydrolase related to dienelactone hydrolase; nuclear localization sequence binding protein; SWI SNF complex protein; GTP-binding protein ypt1; ATPase, F0 complex, subunit H; metal resistance protein ycf1; outer membrane protein, MIM1/TOM13, mitochondrial; ubiquitin-protein ligase molybdopterin-converting factor; GTP-binding protein; predicted mitochondrial carrier protein; 28 kda golgi snare protein; dead-domain-containing protein; trehalose-phosphate synthase (UDP-forming); ran protein binding protein; pkinase-domain-containing protein; ribosome recycling factor domain; phosphatase; nucleic acid-binding, GB-fold; ATP-dependent RNA helicase dbp5; mRNA export protein (contains WD40 repeats); protein phosphatase 2A regulatory subunit A and related proteins; glutaminyl-tRNA synthetase; prolactin regulatory element-binding protein/protein transport protein SEC12p; ribosome assembly protein; C4-type Zn-finger protein; exosomal 3′-5′ exoribonuclease complex subunit Rrp40; transcription regulator HTH, APSES-type DNA-binding domain; RIB7, arfC; 60s ribosomal protein 112; guanylate kinase; predicted membrane protein; glycerol-3-phosphate o-acyltransferase; cactin; translation initiation factor eif3 subunit; biotin holocarboxylase synthetase/biotin-protein ligase; 60s ribosomal protein 123; Inositol monophosphatase; RAS-domain-containing protein; maltase glucoamylase and related hydrolases, glycosyl hydrolase family 31; ribosomal protein S24/S35, mitochondrial, conserved domain; peptide methionine sulfoxide reductase; NAD-dependent formate dehydrogenase; molecular chaperone (DnaJ superfamily); immunoglobulin-like fold; translational repressor pumilio/PUF3 and related RNA-binding proteins (PUF superfamily); urease accessory protein; modular protein with glycoside hydrolase family 13 and glycosyltransferase family 5 domains; orotidine-5-phosphate decarboxylase; phosphoprotein/predicted coiled-coil protein; nucleosome remodeling subunit cafl nurf55 msi1; zinc finger, RING/FYVE/PHD-type; prefoldin subunit 6, KE2 family; thioredoxin h; ADF-like domain-containing protein; alcohol dehydrogenase, class V; 60s ribosomal protein 113; glycoside hydrolase family 3 protein; delta 9 fatty acid desaturase; predicted regulator of rRNA gene transcription (MYB-binding protein); regulator of ribosome synthesis; hexose transport-related protein; protein-histidine kinase; DNA-directed RNA polymerase II subunit I; inositol-3-phosphate synthase; protein transport protein sec22; taurine catabolism dioxygenase TauD/TfdA; ATPase inhibitor, IATP, mitochondria; and glycoside hydrolase family 32 protein.

In embodiments, the isolated protein of the second aspect is of a protein classification selected from: ferredoxin/adrenodoxin reductase; cytochrome; ATP synthase; NADH dehydrogenase; fatty acid desaturase; Acyl-CoA-oxidase; pantothenate kinase; polyphosphate multikinase; G protein-coupled receptor; and succinate dehydrogenase.

In embodiments, the isolated protein of the second aspect is selected from ferredoxin/adrenodoxin reductase; mitochondrial cytochrome b2; cytochrome b; cytochrome c oxidase subunit 1; ATP synthase subunit 6; NADH dehydrogenase subunit 4; cytochrome c oxidase subunit 2; cytochrome c oxidase subunit 3; NADH dehydrogenase subunit 2; NADH dehydrogenase subunit 5; NADH dehydrogenase subunit 6; cytochrome c oxidase subunit 3; delta 9 fatty acid desaturase; Acyl-CoA-oxidase; pantothenate kinase PanK; geranylgeranyl pyrophosphate synthase; fumarate reductase; sucrose transporter; inositol polyphosphate multikinase, ARGR transcription regulatory complex component; G protein-coupled receptor, rhodopsin-like; succinate dehydrogenase; and ATP synthase subunit mitochondrial.

A third aspect of the invention provides a method of modifying a nucleic acid or protein, including a step of changing one or more nucleotides or amino acids of the nucleic acid or protein, to produce:
an isolated nucleic acid comprising a nucleotide sequence set forth in SEQ ID NOs:1240-12684 or a nucleotide sequence at least 80% identical thereto, or a fragment of the isolated nucleic acid, or
an isolated protein encoded by an isolated nucleic acid comprising a nucleotide sequence set forth in SEQ ID NOs:1240-12684 or a nucleotide sequence at least 80% identical thereto, or a fragment or derivative of the isolated protein.

Suitably, the method of the third aspect is a method of producing the isolated nucleic acid of the first aspect or the isolated protein of the second aspect.

In embodiments, the method of modifying the nucleic acid or protein according to the third aspect is a method of mutagenising the nucleic acid or protein.

A fourth aspect of the invention provides a nucleic acid vector or construct comprising the isolated nucleic acid of the first aspect.

In embodiments, the vector or construct of the fourth aspect is an expression vector or construct. In embodiments, the vector or construct is adapted for protein expression in yeast.

In embodiments, the vector or construct of the fourth aspect is a silencing vector or construct. In embodiments, the vector or construct is adapted for gene silencing in yeast.

In embodiments, the vector or construct of the fourth aspect is an editing construct. In embodiments, the editing construct is adapted for gene editing in yeast.

A fifth aspect of the invention provides a cell comprising the nucleic acid of the first aspect, the protein of the second aspect, or the vector or construct of the fourth aspect.

In embodiments, the cell of the fifth aspect is a prokaryotic cell. The prokaryotic cell may be a bacterial cell. In embodiments, the bacterial cell is a Paracoccus cell. The Paracoccus cell may be Paracoccus carotinifaciens.

In embodiments, the cell is a eukaryotic cell. The eukaryotic cell may be selected from a plant cell, an animal cell, an algal cell, and a fungal cell.

In embodiments, the algal cell is a microalgae cell. The microalgae cell may be a Haematococcus cell. In embodiments, the Haematococcus cell is Haematococcus pluvialis.

In embodiments the fungal cell is a yeast cell. The yeast cell may be a Xanthophyllomyces cell. In embodiments, the Xanthophyllomyces cell is Xanthophyllomyces dendrorhous.

A sixth aspect of the invention provides an isolated organism comprising the cell of the fifth aspect. Suitably, the isolated organism is selected from a bacterial strain, algal strain, fungal strain, yeast strain, plant, or animal In embodiments, the organism of the sixth aspect is a yeast strain. In embodiments, the yeast strain is a Xanthophyllomyces dendrorhous strain.

A seventh aspect of the invention provides a method of producing astaxanthin including a step of expressing the isolated nucleic acid of the first aspect or the isolated protein of the second aspect, to thereby produce the astaxanthin.

In embodiments, the expression of the nucleic acid or protein according to the method of the seventh aspect is in vitro expression.

In embodiments, the expression of the nucleic acid or protein according to the method of the seventh aspect is in vivo expression.

An eighth aspect of the invention provides a method of producing astaxanthin including a step of performing metabolism with the cell of the fifth or the organism of the sixth aspect, to thereby produce the astaxanthin.

In embodiments, the step of performing metabolism according to the eighth aspect is a step of performing fermentation with the cell of the fifth aspect or the organism of the sixth aspect.

In embodiments, the cell according to the method of the eighth aspect is Xanthophyllomyces dendrorhous cell.

In embodiments, the method of the eighth aspect includes a step of combining the cell of the fifth aspect or the organism of the sixth aspect with one or more metabolites.

In embodiments, the one or more metabolites combined with the cell or organism comprise a nitrogen source metabolite. The nitrogen source metabolite may be selected from urea, ammonium sulphate, yeast extract, malt extract, bactopeptone, and dried corn steep liquor. In embodiments, the nitrogen source metabolite is or comprises malt extract.

In embodiments, the one or more metabolites combined with the cell or organism comprise a carbon source metabolite. The carbon source metabolite may be selected from molasses, glucose, glycerol, and sucrose. In embodiments, the carbon source metabolite is or comprises molasses.

In a ninth aspect, the invention provides astaxanthin produced according to the method of the seventh or eighth aspect.

In a tenth aspect, the invention provides a non-astaxanthin by-product of the method of the seventh or eighth aspect. In embodiments, the by-product is an invertase enzyme.

In an eleventh aspect, the invention provides a formulation comprising the cell of the fifth aspect or a part thereof, the organism of the sixth aspect or a part thereof, the astaxanthin of the ninth aspect, and/or the by-product of the tenth aspect.

In a twelfth aspect, the invention provides a method of supplementing an animal with the astaxanthin of the ninth aspect or the formulation of the eleventh aspect.

In embodiments, the animal supplemented according to the method of the twelfth aspect is a farmed animal. The farmed animal may be an aquaculture animal. In embodiments, the aquaculture animal according to the method of the ninth aspect is a crustacean or a fish. The crustacean may be selected from shrimp, krill, crab, and crayfish. The fish may be selected from salmon and trout.

In embodiments, the animal supplemented according to the method of the twelfth aspect is a domestic animal or a companion animal. The domestic animal or companion animal may be selected from a canine animal (e.g. a dog), a feline animal (e.g. a cat), and an equine animal (e.g. a horse).

In embodiments, the animal supplement according to the method of the twelfth aspect is a human.

A thirteenth aspect of the invention provides a method of treating or preventing a disease or disorder in a subject, including a step of administering the astaxanthin of the ninth aspect or the formulation of the eleventh aspect to the subject.

Suitably, the subject according to the thirteenth aspect is an animal subject. In embodiments, the subject according to the method of the thirteenth aspect is a human subject.

A fourteenth aspect of the invention provides a method of co-cultivating the cell of the fifth aspect or the organism of the sixth aspect with a further cell or organism. In embodiments of the method of the fourteenth aspect, the cell of the fifth aspect or the organism of the sixth aspect is a yeast cell or organism, and the further cell organism is an algal cell or organism. In embodiments, the cell of the fifth aspect is a Xanthophyllomyces dendrorhous cell or the organism of the sixth aspect is Xanthophyllomyces dendrorhous.

A fifteenth aspect of the invention provide a co-cultivated cell or organism produced according to the method of the fourteenth aspect. In embodiments, the co-cultivated cell or organism is a yeast cell or organism. In embodiments, the co-cultivated cell is aXanthophyllomyces dendrorhous cell or the co-cultivated organism is Xanthophyllomyces dendrorhous.

### BRIEF DESCRIPTION OF THE FIGURES

In order that the invention may be readily understood and put into practical effect, typical embodiments will now be described by way of example with reference to the accompanying figures, wherein:

FIG. 1 sets forth a schematic of methodology developed to obtain and analyse mutant strains of X. dendrorhous producing enhanced AX.

FIG. 2 sets forth relative carotenoid production (as a multiple of wild-type production) in mutant strains of X. dendrorhous identified using screening with antimycin.

FIG. 3 sets forth relative carotenoid production (as a multiple of wild-type production) in mutant strains of X. dendrorhous identified using screening with β-ionone.

FIG. 4 sets forth relative carotenoid production (as a multiple of wild-type production) in mutant strains of X. dendrorhous identified using screening with diphenyamine.

FIG. 5 sets forth relative carotenoid production (as a multiple of wild-type production) in mutant strains of X. dendrorhous identified using screening with flow cytometry and YM plates. Strains MYM0 to MYM13 and MYM18 to MYM40 screened with NTG-FACS; Strains MYM16 to MYM17 UV-FACS and MYM42-MYM55 screened with UV-FACS.

FIG. 6 sets forth relative carotenoid production (as a multiple of wild-type production) in mutant strains of X. dendrorhous identified using screening with flow cytometry and YM plates. Strains MYM56-MYM65 screened with UV-FACS; strains MYM66 to MYM89 screened with MS-FACS; strains MYM91 to MYM96 screened with NTG-FACS.

FIG. 7 sets forth relative carotenoid production in shake flask culture (as a multiple of wild-type production) in fifteen selected mutant strains of X. dendrorhous. Strains with the index MYM were isolated using FACS-YM screening; strains with the index MAMY were isolated using YM supplemented with antimycin; strains with the index MB were isolated using YM plates supplemented with β-ionone; strains with the index MDHA were isolated using YM plates supplemented with diphenylamine.

FIGS. 8 and 9 set forth kinetic model validation for fermentation using mutant X. dendrorhous strain MYM0 in batch culture. Circle=experimental biomass; square=experimental carotenoids; rhombus=experimental sugars. Smooth black line=simulated biomass; smooth light grey line=simulated carotenoids; smooth grey line=simulated sugars.

FIGS. 10 and 11 set forth kinetic model validation for fermentation using mutant X. dendrorhous strain MYM0 in fed-batch culture. Circle=experimental biomass; square=experimental carotenoids; rhombus=experimental sugars. In FIG. 10, smooth black line=simulated biomass; smooth light grey line=simulated carotenoids; smooth grey line=simulated sugars. In FIG. 11, smooth black line=simulated flow rate; smooth grey line=simulated volume.

FIG. 12 sets forth a schematic of variants detected across the genomes of the sequenced mutant strains and the re-sequenced X. dendrorhous CBS 6938 strain. Conserved mutations across the selected mutant strains, but not in the re-sequenced X. dendrorhous CBS 6938 strain, are shown as ‘Shared’.

FIG. 13 sets forth transmembrane helices (as predicted using TMHMM Server 2.0) of a mutant GPCR protein with a premature stop codon identified in all sequenced mutant strains of X. dendrorhous CBS 6938. A. Protein in wild-type X. dendrorhous CBS 6938. B. Protein in sequenced mutant strains of X. dendrorhous CBS 6938.

FIGS. 14 to 17 set forth fermentation data for the mutant X. dendrorhous strain BPAX-A1 (MYM0) and wild-type X. dendrorhous strain CBS6938. The data represent the average of three biological replicates. FIG. 14=fermentation profile for X. dendrorhous BPAX-A1; FIG. 15=specific rates for X. dendrorhous BPAX-A1; FIG. 16=fermentation profile for X. dendrorhous CBS6938; FIG. 17=specific rates for X. dendrorhous CBS6938. In FIG. 14 and FIG. 16: orange line and circle=glucose; blue line and circle=biomass; red line and circle=total carotenoids; red line and triangle=β-carotene; red line and asterisk=cantaxanthin; red line and square=astaxanthin. In FIG. 15 and FIG. 17: orange circle=glucose; blue circle=biomass; red circle=total carotenoids; red triangle=β-carotene; red asterisk=cantaxanthin; red square=astaxanthin.

FIG. 18 sets forth principal component analyses of metabolites at four growth phases of wild-type X. dendrorhous strain CBS6938 and mutant X. dendrorhous strain BPAX-A1 (MYM0).

FIG. 19 sets forth metabolite profile heat maps for X. dendrorhous BPAX-A1 (MYM0). For each metabolite, the response ratio of mutant strain to wild-type strain (X. dendrorhous CBS6938) was normalized to Log 2. Each column represents one of the growth phases studied: Phase 1, Phase 2, Phase 3, and Phase 4—these phases representing points across the kinetic of AX production. Blue colour indicates decreased expression and red colour indicates increased expression across a heat map representation as shown in FIG. 19D. Amino acid (FIG. 19A), fatty acid (FIG. 19C) and central carbon (FIG. 19B) metabolites were assessed.

FIG. 20 sets forth metabolic pathway representation of the central carbon metabolism, carotenoid biosynthesis, and electron transport chain. Bar charts represent metabolite abundance normalized to 100 across Phase 1, Phase 2, Phase 3, and Phase 4 or RNA-seq data in FPKM for Phase 3. Red bar=metabolites in the mutant strain X. dendrorhous BPAX-A1 (MYM0); Light blue bar=metabolites in the wild-type strain X. dendrorhous strain CBS6938; Dark red bar=transcripts in the mutant strain X. dendrorhous BPAX-A1 (MYM0); Green bar=transcripts in the wild-type strain X. dendrorhous strain CBS6938. An asterisk below a bar indicates statistical significance (p<0.05) for metabolites or (q<0.05) for transcripts.

FIG. 21 sets forth fatty acid profile for exemplary AX-containing formulations as described in Example 3 herein.

FIG. 22 sets forth a volcano plot of a comparison of protein expression between X. dendrorhous strain BPAX-A1 (MYM0) and wild-type X. dendrorhous strain CBS6938 during the Phase 3 growth phase. Dashed line is adjusted p-value cutoff (0.05). Blue circles are down-regulated proteins. Red circles are up-regulated proteins. A subset of CDS IDs corresponding to the differentially regulated proteins are given—the full list of CDS IDs for differentially regulated proteins is provided in Table 18.

FIG. 23 sets forth a volcano plot of a comparison of protein expression between X. dendrorhous strain BPAX-A1 (MYM0) and wild-type X. dendrorhous strain CBS6938 during the Phase 4 growth phase. Dashed line is adjusted p-value cutoff (0.05). Blue circles are down-regulated proteins. Red circles are up-regulated proteins. A subset of CDS IDs corresponding to the differentially regulated proteins are given—the full list of CDS IDs for differentially regulated proteins is provided in Table 19.

### BRIEF DESCRIPTION OF THE SEQUENCE LISTING

SEQ ID NOs:1-1239: Genomic sequence contigs from re-sequencing of a sample of X. dendrorhous CBS 6938.

SEQ ID NOs:1240-2795: Genomic sequence contigs of mutant X. dendrorhous strain MAMY3.

SEQ ID NOs:2796-4360: Genomic sequence contigs of mutant X. dendrorhous strain MAMY6.

SEQ ID NOs:4361-5500: Genomic sequence contigs of mutant X. dendrorhous strain MB18.

SEQ ID NOs:5501-6921: Genomic sequence contigs of mutant X. dendrorhous strain MB24.

SEQ ID NOs:6922-8057: Genomic sequence contigs of mutant X. dendrorhous strain MYM0.

SEQ ID NOs:8058-9311: Genomic sequence contigs of mutant X. dendrorhous strain MYM6.

SEQ ID NOs:9312-11033: Genomic sequence contigs of mutant X. dendrorhous strain MYM44.

SEQ ID NOs:11034-12684: Genomic sequence contigs of mutant X. dendrorhous strain MYM92.

SEQ ID NOs:12685-12950: Genomic sequence scaffolds of X. dendrorhous CBS 6938 (ATCC 96594) as published by Sharma et al. BMC genomics 16.1 (2015): 1-13 and publicly available via fungi.ensembl.org/info/data/ftp/index.html.

SEQ ID NOs:12951-19331: CDS sequences of X. dendrorhous CBS 6938 (ATCC 96594) as published by Sharma et al. BMC genomics 16.1 (2015): 1-13 and publicly available via fungi.ensembl.org/info/data/ftp/index.html.

### Biological Deposits

The following biological deposits are incorporated herein under the provisions of the Budapest Treaty. To avoid doubt, this incorporation is not, and is not to be interpreted as, a suggestion that the incorporated deposits are required to work the invention.

CBS 145279: Phaffia rhodozyma BPAX-A1 (alternatively referred to herein as Xanthophyllomyces dendrorhous MYM0) deposited with Westerdijk Fungalbio Diversity Institute, an International Depository Authority at Uppsalalaan 8, 3584 CT Utrecht, Netherlands, on 6 Dec. 2018.

CBS 145280: Phaffia rhodozyma BPAX-A2 (alternatively referred to herein as Xanthophyllomyces dendrorhous MYM92) deposited with Westerdijk Fungalbio Diversity Institute, an International Depository Authority at Uppsalalaan 8, 3584 CT Utrecht, Netherlands, on 6 Dec. 2018.

### DETAILED DESCRIPTION

This invention is at least partly predicated on the identification of mutations in yeast associated with increased accumulation of astaxanthin.

One aspect of the invention is directed to isolated nucleic acids. In typical embodiments, the isolated nucleic acids are associated with increased accumulation of astaxanthin, as herein described.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

The term “nucleic acid” as used herein designates single or double-stranded DNA and RNA. DNA includes genomic DNA and cDNA. RNA includes mRNA, RNA, RNAi, siRNA, cRNA and autocatalytic RNA. Nucleic acids may also be DNA-RNA hybrids. A nucleic acid comprises a nucleotide sequence which typically includes nucleotides that comprise an A, G, C, T or U base. However, nucleotide sequences may include other bases such as inosine, methylycytosine, methylinosine, methyladenosine and/or thiouridine, although without limitation thereto.

A “polynucleotide” is a nucleic acid having eighty (80) or more contiguous nucleotides, while an “oligonucleotide” has less than eighty (80) contiguous nucleotides.

A “probe” may be a single or double-stranded oligonucleotide or polynucleotide, suitably labelled for the purpose of detecting complementary sequences in northern or Southern blotting, for example.

A “primer” is usually a single-stranded oligonucleotide, typically having 15-50 contiguous nucleotides, which is capable of annealing to a complementary nucleic acid ‘template’ and being extended in a template-dependent fashion by the action of a DNA polymerase such as Taq polymerase, RNA-dependent DNA polymerase or Sequenase™.

A typical embodiment of this aspect provides an isolated nucleic acid comprising a nucleotide sequence set forth in SEQ ID NOs:1240-12684 or a nucleotide sequence at least 80% identical thereto, or a fragment of the isolated nucleic acid.

Reference is made herein to sequence identity, in the context of comparisons of nucleotide and/or amino acid sequences. Terms used generally herein to describe sequence relationships between respective sequences of nucleic acids and proteins may include “comparison window”, “sequence identity”, “percentage of sequence identity” and “substantial identity”, and the like.

Because respective nucleic acids/proteins or sequences thereof may each comprise (1) only one or more portions that are shared, and (2) one or more portions which are divergent between the nucleic acids/proteins, sequence comparisons are typically performed by comparing sequences over a “comparison window” to identify and compare local regions of sequence similarity. A “comparison window” refers to a conceptual segment of typically 6, 9 or 12 contiguous residues that is compared to a reference sequence. The comparison window may comprise additions or deletions (i.e., gaps) of about 20% or less as compared to the reference sequence for optimal alignment of the respective sequences.

Optimal alignment of sequences for aligning a comparison window may be conducted by computerised implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, WI, USA) or by inspection, and the best alignment (i.e., resulting in the highest percentage homology over the comparison window) generated by any of the various methods selected. Reference also may be made to the BLAST family of programs as for example disclosed by Altschul et al, 1997, Nucl. Acids Res. 25 3389. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999). Other assessment of sequence alignment approaches can be found, for example, in Thompson et al, 2011, PLOS ONE. 6 (3): e18093, and Nuin et al, 2006, BMC Bioinformatics. 7: 471.

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, U) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program, Version 2.5 for Windows, as was made available from Hitachi Software engineering Co., Ltd., South San Francisco, California, USA.

Typically, the isolated nucleic acid according to this aspect comprises a nucleotide sequence at least about 85%, 90%, 91%, 92%, 93%, 94%, 95%, 96%, 97%, 98%, or 99% identical to a nucleotide sequence set forth in SEQ ID NOs:1240-12684.

As set out hereinabove, this aspect is also directed to fragments of the isolated nucleic acid. More generally, reference is made herein to fragments of nucleic acids and proteins. It will be understood that, as used herein, a nucleic acid or protein “fragment” includes a nucleotide sequence or amino acid sequence, respectively, of less than 100% of that of the full nucleic acid or protein, respectively.

The nucleic acid or protein fragment may comprise about, or at least about, 10, 20, 50, 100, 150, 200, 300, 400, 500, 600, 700, 800, 900, 1000, 1500, 2000, 5000, or 10000 contiguous nucleotides or amino acids of the nucleic acid or protein, respectively. In embodiments, the nucleic acid or protein fragment comprises about, or at least about, 1%, 2%, 3%, 4%, 5%, 10%, 20%, 30%, 40%, 50%, 60%, 70%, 80%, or 90-99% of the full length of the nucleic acid or protein, respectively.

Suitably, the nucleotide sequence of the isolated nucleic acid of this aspect is not identical to any one of the nucleotide sequences set forth in SEQ ID NOs:12685-12950.

The skilled person will appreciate that SEQ ID NOs:12685-12950 correspond to the published genome sequence of the sequenced wild-type yeast strain Xanthophyllomyces dendrorhous CBS 6938 as reported in Sharma et al. BMC genomics 16.1 (2015): 1-13.

Suitably, the nucleotide sequence of the isolated nucleic acid of this aspect is not identical to any one of the nucleotide sequences set forth in SEQ ID NOs:12951-19331.

The skilled person will appreciate that SEQ ID NOs:12951-19331 correspond to the published CDS annotations of the sequenced wild-type yeast strain Xanthophyllomyces dendrorhous CBS 6938 as reported in Sharma et al. BMC genomics 16.1 (2015): 1-13.

Typically, the nucleotide sequence of the isolated nucleic acid according to this aspect is not identical to any one of the nucleotide sequences set forth in SEQ ID NOs:1-1239.

As set out in Example 1, SEQ ID NOs:1-1239 correspond to the re-sequenced genome of wild-type Xanthophyllomyces dendrorhous CBS 6938.

The skilled person will appreciate that various annotations for Xanthophyllomyces dendrorhous CBS 6938 are publicly available as at the filing date, including those annotations hosted at fungi.ensembl.org/info/data/ftp/index.html. To avoid doubt, all such publicly available annotation data is incorporated herein in full, by reference.

In some typical embodiments, the nucleotide sequence of the isolated nucleic acid of this aspect is a variant of a nucleotide sequence set forth in SEQ ID NOs: 12685-19331.

As used herein, a nucleotide or amino acid sequence “variant” will be understood to have one or more nucleotides or amino acids changes, respectively, inclusive of substitutions and deletions. Said changes may be referred to as “variations” of the nucleotide or amino acid sequence.

Typically, variants of a nucleotide sequence or amino acid sequence share at least about 70% or 75%, more typically at least about 80% or 85%, and even more typically at least about 90%, 91%, 92%, 93%, 94%, 95%, 96%, 97%, 98%, or 99% sequence identity with the nucleotide sequence or amino acid sequence.

In some embodiments, a nucleic acid comprising a variant of a nucleotide sequence will hybridize to isolated nucleic acids comprising the nucleotide sequence, under at least low stringency conditions, typically under at least medium stringency conditions, more typically under high stringency conditions.

“Hybridize” and “hybridization” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing between complementary purines and pyrimidines as is well known in the art. In this regard, it will be appreciated that modified purines (for example, inosine, methylinosine and methyladenosine) and modified pyrimidines (thiouridine and methylcytosine) may also engage in base pairing.

“Stringency” as used herein, refers to temperature and ionic strength conditions, and presence or absence of certain organic solvents and/or detergents during hybridisation. The higher the stringency, the higher will be the required level of complementarity between hybridizing nucleotide sequences.

In general terms, “high stringency conditions” designate those conditions under which only nucleic acid having a high frequency of complementary bases will hybridize.

Reference herein to high stringency conditions includes and encompasses:
(i) from at least about 31% v/v to at least about 50% v/v formamide and from at least about 0.01 M to at least about 0.15 M salt for hybridisation at 42° C., and at least about 0.01 M to at least about 0.15 M salt for washing at 42° C.;
(ii) 1% BSA, 1 mM EDTA, 0.5 M NaHP0 4 (pH 7.2), 7% SDS for hybridization at 65° C., and (a) 0.1×SSC, 0.1% SDS; or (b) 0.5% BSA, 1 mM EDTA, 40 mM NaHPO₄ (pH 7.2), 1% SDS for washing at a temperature in excess of 65° C. for about one hour; and
(iii) 0.2×SSC, 0.1% SDS for washing at or above 68° C. for about 20 minutes.

In general, washing is carried out at Tₘ=69.3+0.41 (G+C) %−12° C. In general, the Tₘ of a duplex DNA decreases by about 1° C. with every increase of 1% in the number of mismatched bases.

Notwithstanding the above, stringent conditions are well known in the art, such as described in Chapters 2.9 and 2.10 of Ausubel et al, supra. The skilled person will also recognize that various factors can be manipulated to optimize the specificity of the hybridization. Optimization of the stringency of the final washes can serve to ensure a high degree of hybridization.

It will be appreciated by the skilled person that isolated nucleic acids comprising a variant nucleotide sequence may be producing using a nucleic acid amplification technique. Suitable nucleic acid amplification techniques are well known to the skilled person and include polymerase chain reaction (PCR), strand displacement amplification (SDA), rolling circle replication (RCR), nucleic acid sequence-based amplification (NASBA), Q-β replicase amplification, and helicase-dependent amplification, although without limitation thereto.

As used herein, an “amplification product” refers to a nucleic acid product generated by nucleic acid amplification.

Particularly for analytical purposes, nucleic acid amplification techniques may include quantitative and semi-quantitative techniques such as qPCR, real-time PCR and competitive PCR, as are well known in the art.

Suitably, isolated nucleic acids comprising a variant of a nucleotide sequence may be produced using nucleic acid amplification techniques using one or more degenerate primers based on, or derived from, an isolated nucleic acid comprising the nucleotide sequence.

It is well understood in the art that some nucleotide changes do not change an encoded amino acid sequence (“synonymous” changes), while some nucleotide sequences change an encoded amino acid sequence (“non-synonymous” changes). In some typical embodiments, the nucleotide sequence of the isolated nucleic acid is a variant of a nucleotide sequence set forth in SEQ ID NOs: 12685-19331 comprising a non-synonymous nucleotide change.

It is well understood in the art that some amino acid changes do not substantially change affect activity (“conservative” changes), while some amino acid changes substantially affect protein activity (“non-conservative” changes). In some typical embodiments, the nucleotide sequence of the isolated nucleic acid is a variant of a nucleotide sequence set forth in SEQ ID NOs: 12685-19331 encoding a non-conservative amino acid change.

It will be further understood that nucleotide and/or amino acid variations may be more specifically characterised in a range of ways as will be known to the skilled person. For the purposes of this invention, nucleotide changes may (by way of non-limiting example) be characterised with reference to a gene or CDS region as: ‘synonymous’ (resulting in no change to an encoded amino acid sequence); ‘missense’ (resulting in a change to an encoded amino acid sequence); ‘upstream’ (occurring upstream of the gene or CDS); ‘upstream less than or equal to 100 base pairs’ (occurring within 100 base pairs of the start of the gene or CDS), ‘downstream’ (occurring downstream of the gene or CDS), ‘downstream less than or equal to 100 bp’ (occurring within 100 base pairs of the end of the gene or CDS); ‘intergenic’ (occurring in a region between respective genes or CDSs); ‘intron variant’ (occurring in an intron of the gene or unspliced sequence containing the CDS); ‘splice variant’ (causing a variation in splicing of the gene or unspliced sequence containing the CDS); ‘stop gained’ (introducing a stop codon into the gene or CDS); ‘start lost’ (removing a start codon from the gene or CDS).

In some typical embodiments, the nucleotide sequence of the isolated nucleic acid is a variant of a nucleotide sequence set forth in SEQ ID NOs: 12685-19331 characterised as ‘synonymous’, ‘missense’, ‘upstream’, ‘upstream less than or equal to 100 base pairs’, ‘downstream’, ‘downstream less than or equal to 100 bp’, ‘intergenic’, ‘intron variant’, ‘splice variant’, ‘stop gained’, or ‘start lost’, with reference to one or more suitable genes or CDSs.

Tables 12, 13, 14, and 15 set out hereinbelow show nucleotide sequence variations identified relative to genomic scaffolds published by Sharma et al. BMC genomics 16.1 (2015): 1-13, which scaffolds are incorporated herein as SEQ ID NOs:12685-12950, as hereinabove described. CDS sequences as published by Sharma et al. BMC genomics 16.1 (2015): 1-13 in or near to which the nucleotide sequence variations are located (as applicable) are also shown in Tables 12-15, which CDS sequences are incorporated herein as SEQ ID NOs:12951-19331, as hereinabove described.

With reference to Sharma et al. BMC genomics 16.1 (2015): 1-13, SEQ ID NOs:12685-12950, and SEQ ID NOs:12951-19331, the skilled person will readily appreciate that each respective nucleotide sequence variation as set out in Tables 12-15 can be characterised as a variation of:
a genic region, i.e. a region from the transcription start site to the end of the 3′ UTR for a given gene;
a regulatory region, i.e. a region associated with regulation of expression of a given gene; or
an intergenic region, i.e. a region that is not a genic or regulatory region.

By way of example, as per Table 12:
variation X₂₂₃ is a variation of a nucleotide sequence in a genic region of CDS CED80056;
variation X₂₄₆ is a variation of a nucleotide sequence in a regulatory region for CDS CED80058;
variation X₂₆₀ is a variation of a nucleotide sequence in an intergenic region located towards the start of genomic scaffold 7 as per Sharma et al, supra.

By way of another example, as per Table 13:
variation Y₂₄ is a variation of a nucleotide sequence in a genic region of CDS CED80060;
variation Y₃₂₂ is a variation of a nucleotide sequence in a regulatory region for CDS CED85243.

By way of another example, as per Table 14:
variation Z₉ is a variation of a nucleotide sequence in a genic region of CDS CDZ96153;
variation Z₂₄ is a variation of a nucleotide sequence in a regulatory region for CDS CED83975.

By way of another example, as per Table 15:
variation V₃ is a variation of a nucleotide sequence in a genic region of CDS CDZ98193;
variation V₂₃ is a variation of a nucleotide sequence in a regulatory region for CDZ96151.

It will be further appreciated that variations as set forth in Tables 12-15 may be more specifically characterised, such as in the manner hereinabove described including characterisations such as synonymous, missense, upstream, downstream, intergenic, intron variant, splice variant, stop gained, or start lost. Characterisation in said (or similar) manner is provided in Tables 12-15.

With reference to Example 1 and Table 12, it will be appreciated that variations X₁-X₂₂₂ are variations identified in the re-sequenced wild-type Xanthophyllomyces dendrorhous strain relative to the published genome sequence in Sharma et al., supra.

In some typical embodiments, the nucleotide sequence of the isolated nucleic acid of this aspect is a variant of a genic, regulatory, or intergenic sequence as set out in Table 12. The nucleotide sequence variant may be associated with a change in an encoded amino acid sequence and/or expression of a CDS sequence set out in Table 12.

In some typical embodiments, the nucleotide sequence of the isolated nucleic acid of this aspect is a variant of a genic, regulatory, or intergenic region as set out in Table 13. The nucleotide sequence variant may be associated with a change in an encoded amino acid sequence and/or expression of a CDS sequence set out in Table 13.

In some typical embodiments, the nucleotide sequence of the isolated nucleic acid of this aspect is a variant of a genic, regulatory, or intergenic region as set out in Table 14. The nucleotide sequence variant may be associated with a change in an encoded amino acid sequence and/or expression of a CDS sequence set out in Table 14.

In some typical embodiments, the nucleotide sequence of the isolated nucleic acid of this aspect is a variant of a genic, regulatory, or intergenic region as set out in Table 15. The nucleotide sequence variant may be associated with a change in an encoded amino acid sequence and/or expression of a CDS sequence set out in Table 15.

Tables 16 and 17 set out hereinbelow show transcript expression in a mutant Xanthophyllomyces dendrorhous strain (MYM0) relative to the wild-type CBS 6938 strain. In some typical embodiments, the nucleotide sequence of the isolated nucleic acid of this aspect is a variant of a nucleotide sequence encoding a transcript set out in Table 16. In some typical embodiments, the nucleotide sequence of the isolated nucleic acid of this aspect is a variant of a nucleotide sequence encoding a transcript set forth in Table 17.

With reference to Sharma et al. BMC genomics 16.1 (2015): 1-13, SEQ ID NOs:12685-12950, and SEQ ID NOs:12951-19331, at least certain regulatory sequences associated with control of expression of the transcripts set out in Table 16 and Table 17 will be apparent to or readily determinable by the skilled person. The skilled person is further directed to all annotations available at fungi.ensembl.org/info/data/ftp/index.html for the wild-type CBS 6938 strain, incorporated herein by reference.

Accordingly, in some typical embodiments, the nucleotide sequence of the isolated nucleic acid of this aspect is a regulatory sequence for a transcript set out in Table 16. In some typical embodiments, the nucleotide sequence of the isolated nucleic acid of this aspect is a regulatory sequence for a transcript set out in Table 17.

Tables 18 and 19 set out hereinbelow show CDS sequences encoding proteins differentially expressed (based on proteomic analysis) in Xanthophyllomyces dendrorhous strain MYM0 relative to the wild-type CBS 6938 strain. In some typical embodiments, the nucleotide sequence of the isolated nucleic acid of this aspect is a variant of a CDS sequence set out in Table 18 and/or Table 19. The nucleotide sequence variant may be associated with a change in an encoded amino acid sequence and/or expression of a CDS sequence set out in Table 18 and/or Table 19.

With reference to Sharma et al. BMC genomics 16.1 (2015): 1-13, SEQ ID NOs:12685-12950, and SEQ ID NOs:12951-19331, at least certain regulatory sequences associated with control of expression of CDS sequences and encoded proteins set out in Table 18 and Table 19 will be apparent to or readily determinable by the skilled person. The skilled person is further directed to all annotations available at fungi.ensembl.org/info/data/ftp/index.html for the wild-type CBS 6938 strain, incorporated herein by reference.

Accordingly, in some typical embodiments, the nucleotide sequence of the isolated nucleic acid of this aspect is a regulatory sequence for a CDS sequence and/or encoded protein set out in Table 18 and/or Table 19.

In some typical embodiments, the nucleotide sequence of the isolated nucleic acid of this aspect comprises a nucleotide change at a position of one or more variations X₂₂₃-X₈₃₉₅, as set forth in Table 12, relative to SEQ ID NOs:12685-12950. Typically, the nucleotide change comprises a change at a different position than any of the variations X₁-X₂₂₂, as set forth in Table 12, relative to SEQ ID NOs:12685-12950.

In some typical embodiments, the nucleotide sequence of the isolated nucleic acid comprises a nucleotide change of one or more variations X₂₂₃-X₈₃₉₅, as set forth in Table 12, relative to SEQ ID NOs:12685-12950. Typically, the nucleotide change comprises a change that is different than any of the variations X₁-X₂₂₂, as set forth in Table 12, relative to SEQ ID NOs:12685-12950.

In some typical embodiments, the nucleotide sequence of the isolated nucleic acid comprises a nucleotide change at a position of one or more variations Y₁-Y₃₆₈, as set forth in Table 13, relative to SEQ ID NOs:12685-12950.

In some typical embodiments, the nucleotide sequence of the isolated nucleic acid comprises a nucleotide change of one or more variations Y₁-Y₃₆₈, as set forth in Table 13, relative to SEQ ID NOs:12685-12950.

In some typical embodiments, the nucleotide sequence of the isolated nucleic acid comprises a nucleotide change at a position of one or more variations Z₁-Z₂₅, as set forth in Table 14, relative to SEQ ID NOs:12685-12950.

In some typical embodiments, the nucleotide sequence of the isolated nucleic acid comprises a nucleotide change of one or more variations Z₁-Y₂₅, as set forth in Table 14, relative to SEQ ID NOs:12685-12950.

In some typical embodiments, the nucleotide sequence of the isolated nucleic acid comprises a nucleotide change at a position of one or more variations V₁-Z₂₆, as set forth in Table 15, relative to SEQ ID NOs:12685-12950.

In some typical embodiments, the nucleotide sequence of the isolated nucleic acid comprises a nucleotide change of one or more variations V₁-V₂₆, as set forth in Table 15, relative to SEQ ID NOs:12685-12950.

SEQ ID NOs:1240-2795 are genomic sequence contigs of mutant X. dendrorhous strain MAMY3. In some typical embodiments, the isolated nucleic acid comprises a nucleotide sequence set forth in SEQ ID NOs:1240-2795 or a variant thereof, wherein the nucleotide sequence or variant thereof is not identical to any one of the nucleotide sequences set forth in SEQ ID NOs:12685-12950 or SEQ ID NOs:12951-19331. Typically, the nucleotide sequence or variant thereof is not identical to any one of the nucleotide sequences set forth in SEQ ID NOs:1-1239.

SEQ ID NOs:2796-4360 are genomic sequence contigs of mutant X. dendrorhous strain MAMY6. In some typical embodiments, the isolated nucleic acid comprises a nucleotide sequence set forth in SEQ ID NOs:2796-4360 or a variant thereof, wherein the nucleotide sequence or variant thereof is not identical to any one of the nucleotide sequences set forth in SEQ ID NOs:12685-12950 or SEQ ID NOs:12951-19331. Typically, the nucleotide sequence or variant thereof is not identical to any one of the nucleotide sequences set forth in SEQ ID NOs:1-1239.

SEQ ID NOs:4361-5500 are genomic sequence contigs of mutant X. dendrorhous strain MB18. In some typical embodiments, the isolated nucleic acid comprises a nucleotide sequence set forth in SEQ ID NOs:4361-5500 or a variant thereof, wherein the nucleotide sequence or variant thereof is not identical to any one of the nucleotide sequences set forth in SEQ ID NOs:12685-12950 or SEQ ID NOs:12951-19331. Typically, the nucleotide sequence or variant thereof is not identical to any one of the nucleotide sequences set forth in SEQ ID NOs:1-1239.

SEQ ID NOs:5501-6921 are genomic sequence contigs of mutant X. dendrorhous strain MB24. In some typical embodiments, the isolated nucleic acid comprises a nucleotide sequence set forth in SEQ ID NOs:5501-6921 or a variant thereof, wherein the nucleotide sequence is not identical to any one of the nucleotide sequences set forth in SEQ ID NOs:12685-12950 or SEQ ID NOs:12951-19331. Typically, the nucleotide sequence or variant thereof is not identical to any one of the nucleotide sequences set forth in SEQ ID NOs:1-1239.

SEQ ID NOs:6922-8057 are genomic sequence contigs of mutant X. dendrorhous strain MYM0. In some typical embodiments, the isolated nucleic acid comprises a nucleotide sequence set forth in SEQ ID NOs:6922-8057 or a variant thereof, wherein the nucleotide sequence or variant thereof is not identical to any one of the nucleotide sequences set forth in SEQ ID NOs:12685-12950 or SEQ ID NOs:12951-19331. Typically, the nucleotide sequence or variant thereof is not identical to any one of the nucleotide sequences set forth in SEQ ID NOs:1-1239.

SEQ ID NOs:8058-9311 are genomic sequence contigs of mutant X. dendrorhous strain MYM6. In some typical embodiments, the isolated nucleic acid comprises a nucleotide sequence set forth in SEQ ID NOs:8058-9311 or a variant thereof, wherein the nucleotide sequence or variant thereof is not identical to any one of the nucleotide sequences set forth in SEQ ID NOs:12685-12950 or SEQ ID NOs:12951-19331. Typically, the nucleotide sequence or variant thereof is not identical to any one of the nucleotide sequences set forth in SEQ ID NOs:1-1239.

SEQ ID NOs:9312-11033 are genomic sequence contigs of mutant X. dendrorhous strain MYM44. In some typical embodiments, the isolated nucleic acid comprises a nucleotide sequence set forth in SEQ ID NOs:9312-11033 or a variant thereof, wherein the nucleotide sequence is not identical to any one of the nucleotide sequences set forth in SEQ ID NOs:12685-12950 or SEQ ID NOs:12951-19331. Typically, the nucleotide sequence or variant thereof is not identical to any one of the nucleotide sequences set forth in SEQ ID NOs:1-1239.

SEQ ID NOs:11034-12684 are genomic sequence contigs of mutant X. dendrorhous strain MYM92. In some typical embodiments, the isolated nucleic acid comprises a nucleotide sequence set forth in SEQ ID NOs:11034-12684 or a variant thereof, wherein the nucleotide sequence is not identical to any one of the nucleotide sequences set forth in SEQ ID NOs:12685-12950 or SEQ ID NOs:12951-19331. Typically, the nucleotide sequence or variant thereof is not identical to any one of the nucleotide sequences set forth in SEQ ID NOs:1-1239.

Another aspect of the invention provides an isolated protein encoded by the isolated nucleic acid of the preceding aspect, or a fragment, variant, or derivative of the isolated protein.

By “protein” is meant an amino acid polymer. The amino acids may be natural or non-natural amino acids, D- or L-amino acids as are well understood in the art.

A “peptide” is a protein having no more than fifty (50) amino acids.

A “polypeptide” is a protein having more than fifty (50) amino acids.

As used herein, “derivative” proteins have been altered, for example by conjugation or complexing with other chemical moieties, by post-translational modification (e.g. phosphorylation, acetylation etc.), modification of glycosylation (e.g. adding, removing or altering glycosylation) and/or inclusion of additional amino acid sequences as is understood in the art.

Additional amino acid sequences may include fusion partner amino acid sequences which create a fusion protein. By way of example, fusion partner amino acid sequences may assist in detection and/or purification of the isolated fusion protein. Non-limiting examples include metal-binding (e.g. polyhistidine) fusion partners, maltose binding protein (MBP), Protein A, glutathione S-transferase (GST), fluorescent protein sequences (e.g. GFP), epitope tags such as Myc, FLAG and haemagglutinin tags.

For the particular purpose of fusion polypeptide purification by affinity chromatography, relevant matrices for affinity chromatography include glutathione-, amylose-, and nickel- or cobalt conjugated resins respectively. Many such matrices have been made available in kit form, such as the QIAexpress™ system (Qiagen) useful with (HIS6) fusion partners and the Pharmacia GST purification system.

Preferably, the fusion partners also have protease cleavage sites, such as for Factor X or Thrombin, which allow the relevant protease to partially digest the fusion polypeptide of the invention and thereby liberate the recombinant polypeptide of the invention therefrom. The liberated polypeptide can then be isolated from the fusion partner by subsequent chromatographic separation.

Other protein derivatives include, but are not limited to, modification to amino acid side chains, incorporation of unnatural amino acids and/or their derivatives during peptide, polypeptide or protein synthesis and the use of crosslinkers and other methods which impose conformational constraints on the isolated protein, fragments and variants disclosed herein.

In embodiments, the isolated protein of this aspect is of a protein classification selected from: cytochrome c oxidase subunit 1; ATP synthase subunit 6; NADH dehydrogenase; NADH dehydrogenase subunit 4; cytochrome c oxidase subunit 2; NADH dehydrogenase subunits 2, 5; cytochrome b; NADH dehydrogenase subunit 6; cytochrome c oxidase subunit 3; V-SNARE; DNA polymerase zeta; mRNA splicing factor; ubiquitin-conjugating enzyme E2-binding protein; transcription coactivator protein; phosphoenolpyruvate carboxykinase; mercaptopyruvate sulfurtransferase/thiosulfate sulfurtransferase; HLH transcription factor EBF/Olf-1; peptidase S9; capl-related protein; C4-type Zn-finger protein; endonuclease MUS81; p-loop containing nucleoside triphosphate hydrolase protein; inorganic phosphate transporter; nuclear AAA ATPase (VCP subfamily); leucine-trna ligase; xanthine uracil permease; basic-leucine zipper domain protein; DNA-binding centromere protein B (CENP-B); dimeric dihydrodiol dehydrogenase; pre-mRNA splicing factor prp1; ATP-dependent clp protease proteolytic subunit; myosin 5; MYND Zn-finger protein; splicing factor 3b, subunit 2; OCH1; phosphoinositide phosphatase; ATP synthase subunit mitochondrial kinase-like protein; small molecule transporter protein; B-block binding subunit of TFIIIC; pre-mRNA-splicing factor clf1; transcriptional regulator protein; DnaJ superfamily molecular chaperone; atypical pikk frap protein kinase; 1,4-alpha-glucan branching enzyme/starch branching enzyme II; cyclin-dependent kinase regulatory subunit; heat shock protein 60; FOG protein; density-regulated protein related to translation initiation factor 1 (eIF-1/SUI1); CDC12-septin; MRG-domain-containing protein; mitochondrial cytochrome b2; eukaryotic translation initiation factor 2 subunit alpha; Zn-finger protein; DNA mismatch repair protein; ATPase; RNA polymerase II, subunit POLR2C/RPB3; ATP-dependent metallopeptidase HFL; sexual differentiation process protein ISP4; extracellular protein SEL-1; nucleic acid-binding, OB-fold protein; dead-domain-containing protein; 60s ribosomal protein 121; protein phosphatase; regulatory subunit PPP1R3C/D; ATP-dependent RNA helicase dhx8; RmlC-like jelly roll fold protein; white collar 1 protein; UV radiation resistance associated protein; SAP family cell cycle dependent phosphatase-associated protein; exosomal 3′-5′ exoribonuclease complex, subunit Rrp44/Dis3; ABC transporter; peptidase M28; delta 9 fatty acid desaturase; microtubule binding protein; DNA replication factor large subunit; beta and beta-prime subunits of DNA dependent RNA-polymerase; aspartate-semialdehyde dehydrogenase; metalloexopeptidase; acyl-oxidase; E3 ubiquitin ligase; TPR repeat-containing protein; peroxisomal half ABC transporter; nucleosome assembly protein; mRNA cleavage and polyadenylation factor II complex, subunit CFT2 (CPSF subunit); inositol polyphosphate multikinase, component of the ARGR transcription regulatory complex; ATP-dependent RNA helicase DBP5; SMAD/FHA domain; RNA polymerase II transcription initiation/nucleotide excision repair factor TFIIH, subunit TFB2; immunoglobulin-like; pre-mRNA splicing factor; g-protein alpha-subunit; F-box protein containing LRR; DRIM (down-regulated in metastasis)-like proteins; acid phosphatase; extracellular matrix glycoprotein Laminin subunits alpha and gamma; GTP-binding protein ypt1; DNA repair protein; mRNA (guanine-n7-)-methyltransferase; ataxia telangiectasia protein; ornithine decarboxylase antizyme; aldo/keto reductase family proteins; spermidine/spermine synthases family; membrane protein; high-affinity cell membrane calcium channel; WD40/YVTN repeat-like-containing domain; Golgi apparatus membrane protein TVP23; conserved hypothetical protein CHP02453; AAA-type ATPase; HSP20-like chaperone; pantothenate kinase PanK; mediator complex, subunit Med4; M13 family peptidase; Ca-transporting ATPase; histone tail methylase containing SET domain; ribonuclease iii; zinc finger, RING-type; 40s ribosomal protein s15; nuclear export receptor crml; voltage-gated chloride channel; phosphatidylinositol 4-kinase; geranylgeranyl pyrophosphate synthase; membrane coat complex retromer, subunit VPS5/SNX1; iron permease FTR1; t-complex protein alpha subunit (tcp-1-alpha); short chain-type dehydrogenase; polyadenylation factor I complex, subunit, Yth1 (CPSF subunit); lipase; RNA polymerase III, subunit C34; cytoplasm protein; plant ascorbate peroxidase; lysine-tRNA ligase; retrotransposon tyl-copia subclass; GTP-binding protein; ring finger protein; cytochrome b5; tetraspanin/peripherin; snf2 family amino-terminal protein; arginyl-tRNA synthetase; dihydropteroate synthase; START-like domain protein; WD40 repeat-containing protein; alanine-tRNA ligase; protein kinase essential for the initiation of DNA replication; palp-domain-containing protein; arginase deacetylase; ATP-NAD kinase; fumarate reductase; peptidase c1b bleomycin hydrolase; cytochrome c oxidase, subunit IV/COX5b; 1-aminocyclopropane-1-carboxylate synthase, and related proteins; endosomal p24a protein; sirtuin 4 and related class II sirtuins (SIR2 family); protein-tyrosine/dual specificity phosphatase; calcineurin responsive transcription factor przl; acyl-n-acyltransferase; maintenance of telomere capping protein 1, Mtcl; beta-1,6-N-acetylglucosaminyltransferase; Zn(2)-C6 fungal-type DNA-binding domain; nuclear pore complex, Nup155 component; E3 ubiquitin protein ligase; transmembrane protein; major facilitator superfamily domain, general substrate transporter; protein kinase; glucosyltransferase-Alg8p; Golgi-associated protein/Nedd4 WW domain-binding protein; origin recognition complex, subunit 1; cysteine proteinase; related to C2H2 zinc finger protein FLBC; C-14 reductase; hydroxymethylglutaryl-CoA reductase; RNA polymerase II transcription initiation/nucleotide excision repair factor TFIIH, subunit TFB4; microfibrillar-associated protein MFAP1; tetratricopeptide-like helical; SNF2-family ATP dependent chromatin remodeling factor SNF21; arsenical pump-driving atpase; guanylate kinase; fatty acid-2 hydroxylase; sucrose transporter; RNA polymerase I termination factor, MYB superfamily; ferredoxin/adrenodoxin reductase; WD40 repeat-like protein; HSP90 co-chaperone CNS1 (contains TPR repeats); RhoGEF GTPase; vacuole import and degradation protein; mitochondrial Fe2 transporter MMT 1 and related transporters (cation diffusion facilitator superfamily); glycoside hydrolase, superfamily; CLASP N-terminal domain; pinin/SDK/MemA protein; G protein-coupled receptor, rhodopsin-like; histone deacetylase clr6; methylase protein; and RAB protein geranylgeranyltransferase component A.

It will be appreciated that the preceding protein classifications correspond to proteins encoded by one or more nucleotide sequences as herein described with reference to Examples 1 and 2 and Tables 12 and 13.

In some embodiments, the isolated protein of this aspect is of a protein classification selected from: short-chain dehydrogenase; deoxyribodipyrimidine photolyase/cryptochrome; nuclear transport factor 2; 60s ribosomal protein 132; armadillo/beta-catenin-like repeat-containing protein; 60s ribosomal protein 110a; pyridoxamine 5′-phosphate oxidase-like, FMN-binding domain; glutaredoxin-related protein; glycosyl transferase, family 8-glycogenin; mitochondrial carrier; nucleosome assembly protein; sterile alpha motif, type 2; snare protein ykt6; UDP-glucose dehydrogenase; predicted translation factor, contains W2 domain; G-protein beta subunit-like protein; heat shock protein HSS1; 40s ribosomal protein s7; ATP synthase f1 beta subunit; catalase 1; stress responsive alpha-beta barrel; cytokinin riboside 5′-monophosphate phosphoribohydrolase LOG; EF-hand domain pair; 20s proteasome subunit; ferrochelatase; glycine hydroxymethyltransferase; carboxypeptidase s; NADH-ubiquinone oxidoreductase 304 kDa subunit precursor; phytoene dehydrogenase; ribosomal protein L49/IMG2; nop10p-domain-containing protein; thioredoxin/protein disulfide isomerase; predicted dehydrogenase; 6-phosphogluconate dehydrogenase; NADH-dehydrogenase (ubiquinone); COPII vesicle protein; ornithine aminotransferase; ER-associated protein catabolism-related protein; isocitrate dehydrogenase; AAA atpase; probable NADP-dependent dehydrogenase acting on 3-hydroxy acids; CNDP dipeptidase; actin-related protein Arp2/3 complex, subunit ARPC2; branched-chain amino acid aminotransferase ii; carbon-nitrogen hydrolase; aspartate aminotransferase; NADPH oxidase; 26s proteasome subunit p45; pre-mRNA-splicing factor rsel; porphobilinogen deaminase; prolyl oligopeptidase; ABC transporter; 40s ribosomal protein s9; polyadenylate-binding protein; ATP-dependent RNA helicase dhx8; fatty acid synthase complex subunit alpha; glycosyltransferase family 35 protein; WD repeat protein; heat shock protein 60; succinate dehydrogenase; translocase of outer mitochondrial membrane complex, subunit TOM70/TOM72; nucleic acid-binding protein; nucleotide excision repair factor NEF2, RAD23 component; t-complex protein alpha subunit (tcp-1-alpha); k506-binding protein 2; aromatic amino acid aminotransferase; adenylate kinase; alpha-aminoadipate reductase lys1p; coatomer protein subunit alpha; 40s ribosomal protein s21; carbamoyl-phosphate synth; histone acetyltransferase SAGA, TRRAP/TRA1 component, PI-3 kinase superfamily; SAM-dependent RNA methyltransferase; related to 2-hydroxy-3-oxopropionate reductase; transcriptional coactivator p100; 60s ribosomal protein 113a; ornithine carbamoyltransferase; eukaryotic translation initiation factor 5b; aconitate hydratase; RNA 2-o-methyltransferase fibrillarin; t-complex protein beta subunit (tcp-1-beta); voltage-dependent ion-selective channel; coatomer beta subunit; succinate-ligase (adp-forming); carbamoyl-phosphate synthase; related to ste23-metalloprotease involved in a-factor processing; microtubule binding protein; pyridoxalphosphate-dependent enzyme/predicted threonine synthase; fact complex subunit SPT16; SLY1 vesicle trafficking secl-like protein; cytoplasm protein; NADH dehydrogenase; phosphoglycerate kinase; arm repeat-containing protein; ribonuclease III domain; GTP binding protein 4; peptidyl-prolyl cis-trans isomerase b; Translation initiation factor 4F, ribosome/mRNA-bridging subunit (eIF-4G); eukaryotic polypeptide chain release factor 3; asparagine synthase (glutamine-hydrolyzing); splicing factor U2AF, large subunit (RRM superfamily); NADH-cytochrome b5 reductase; histidine biosynthesis trifunctional-protein; Enoyl-CoA hydratase; alcohol; imidazoleglycerol phosphate synthase; thioredoxin-like fold; ef-hand; electron-transferring-flavoprotein dehydrogenase; MDF1-domain-containing protein; transcription factor IIS, N-terminal; heat shock protein 70; pyruvate carboxylase; homoaconitate hydratase; uncharacterized conserved coiled-coil protein; alternative splicing factor SRp55/B52/SRp75 (RRM superfamily); eukaryotic translation initiation factor 3 subunit 7; threonyl-trna synthetase; RmlC-like jelly roll fold; 60s ribosomal protein 120; mRNA splicing factor; pre-mrna-processing protein 45; atp-dependent rma helicase rrp3; dihydrolipoyllysine-residue acetyltransferase; Acyl-CoA synthetase; ribosomal protein S5; phenylalanyl-tRNA synthetase subunit beta; wd40 repeat-like protein; vacuolar ATP synthase subunit d; phosphatidylserine decarboxylase; vigilin; RNA recognition motif domain; plasma membrane h( )-atpase 1; RRM motif-containing protein; predicted GTPase-activating protein; F1-ATP synthase assembly protein; acetyl-hydrolase; peptidyl-prolyl cis-trans isomerase; antiviral helicase; acetyl CoA carboxylase; age pka protein kinase; ATP-dependent RNA helicase pitchoune; Microtubule-associated protein; cell-cycle nuclear protein, contains WD-40 repeats; phosphoserine aminotransferase; vacuolar protein sorting-associated protein; GMP synthase; translational regulator gcn20-like abc transporter; GDP-mannose pyrophosphorylase; acetyl CoA acyltransferase 2; phosphoketolase; delta 12 fatty acid desaturase; vacuolar protein 8; predicted haloacid-halidohydrolase and related hydrolases; class iii adh enzyme; t-complex protein 1; isocitrate lyase; atpase; 6-phosphogluconolactonase; mitochondrial inner membrane protein; t-complex protein 1 subunit delta; adaptor protein complex ap-1 gamma subunit; rRNA processing protein Rrp5; succinate:fumarate antiporter; predicted proline-serine-threonine phosphatase-interacting protein (PSTPIP); phospho-2-dehydro-3-deoxyheptonate aldolase; RNA-binding domain-containing protein; epsilon DNA polymerase; cullins; asparaginyl-tRNA synthetase; dihydroxy-acid dehydratase; SNARE protein SED5/Syntaxin 5; centromere microtubule binding protein cbf5; histidyl-trna synthetase; endoplasmic reticulum protein EP58, contains filamin rod domain and KDEL motif; 3-isopropylmalate dehydrogenase; Glycosyl transferase, family 1; eukaryotic translation initiation factor 3 subunit 6; phosphoglycerate mutase family; chromatin remodelling complex ATPase chain; predicted hydrolases or acyltransferases (alpha/beta hydrolase superfamily); NADH dehydrogenase subunits 2, 5, and related proteins; synaptobrevin-like protein; 40s ribosomal protein s6; ubiquitin C-terminal hydrolase UCHL 1; polyC-binding proteins alphaCP-1 and related KH domain proteins; nucleolar RNA-associated protein (NRAP); WD40 repeat-containing protein; pyruvate decarboxylase; RhoGEF GTPase; Ca2-dependent lipid-binding protein CLB1/vesicle protein vp115/Granuphilin A, contains C2 domain; molecular co-chaperone STI1; vacuolar H-ATPase V1 sector, subunit E; p-loop containing nucleoside triphosphate hydrolase protein; spliceosome subunit; microtubule-binding protein involved in cell cycle control; karyopherin (importin) beta 3; DNA-dependent RNA polymerase ii second largest subunit; coatomer subunit gamma; dehydrogenase kinase; mitochondrial pyruvate dehydrogenase el component beta subunit; glycoside hydrolase family 13 protein; NAD-specific glutamate dehydrogenase; mitochondrial 50s ribosomal protein 13; Ran GTPase-activating protein; FKBP-type peptidyl-prolyl cis-trans isomerase; 60s ribosomal protein 119; small nuclear ribonucleoprotein splicing factor; mannosyltransferase; dUTP pyrophosphatase; GST, gst; glutamate-trna ligase; mov34-domain-containing protein; mitochondrial nuclease; 1,4-benzoquinone reductase-like; thiamine biosynthetic bifunctional enzyme; protein of unknown function DUF3602; upf0041-domain-containing protein; 60s ribosomal protein 111; serine/threonine protein phosphatase 2A, regulatory subunit; argininosuccinate lyase; elongation factor 1 beta delta chain; bar-domain-containing protein; uridylate kinase; phosphatidylethanolamine n-methyltransferase; stomatin family protein; ubiquitin-conjugating enzyme; glycosyltransferase family 2 protein; signal recognition particle protein; B-cell receptor-associated protein and related proteins; RNA-binding S4 domain; Drebrins and related actin binding proteins; small gtpase-binding protein; gtp cyclohydrolase i; ps16 protein; predicted hydrolase related to dienelactone hydrolase; nuclear localization sequence binding protein; SWI SNF complex protein; GTP-binding protein ypt1; ATPase, F0 complex, subunit H; metal resistance protein ycf1; outer membrane protein, MIM1/TOM13, mitochondrial; ubiquitin-protein ligase molybdopterin-converting factor; GTP-binding protein; predicted mitochondrial carrier protein; 28 kda golgi snare protein; dead-domain-containing protein; trehalose-phosphate synthase (UDP-forming); ran protein binding protein; pkinase-domain-containing protein; ribosome recycling factor domain; phosphatase; nucleic acid-binding, GB-fold; ATP-dependent RNA helicase dbp5; mRNA export protein (contains WD40 repeats); protein phosphatase 2A regulatory subunit A and related proteins; glutaminyl-tRNA synthetase; prolactin regulatory element-binding protein/protein transport protein SEC12p; ribosome assembly protein; C4-type Zn-finger protein; exosomal 3′-5′ exoribonuclease complex subunit Rrp40; transcription regulator HTH, APSES-type DNA-binding domain; RIB7, arfC; 60s ribosomal protein 112; guanylate kinase; predicted membrane protein; glycerol-3-phosphate o-acyltransferase; cactin; translation initiation factor eif3 subunit; biotin holocarboxylase synthetase/biotin-protein ligase; 60s ribosomal protein 123; Inositol monophosphatase; RAS-domain-containing protein; maltase glucoamylase and related hydrolases, glycosyl hydrolase family 31; ribosomal protein S24/S35, mitochondrial, conserved domain; peptide methionine sulfoxide reductase; NAD-dependent formate dehydrogenase; molecular chaperone (DnaJ superfamily); immunoglobulin-like fold; translational repressor pumilio/PUF3 and related RNA-binding proteins (PUF superfamily); urease accessory protein; modular protein with glycoside hydrolase family 13 and glycosyltransferase family 5 domains; orotidine-5-phosphate decarboxylase; phosphoprotein/predicted coiled-coil protein; nucleosome remodeling subunit cafl nurf55 msi1; zinc finger, RING/FYVE/PHD-type; prefoldin subunit 6, KE2 family; thioredoxin h; ADF-like domain-containing protein; alcohol dehydrogenase, class V; 60s ribosomal protein 113; glycoside hydrolase family 3 protein; delta 9 fatty acid desaturase; predicted regulator of rRNA gene transcription (MYB-binding protein); regulator of ribosome synthesis; hexose transport-related protein; protein-histidine kinase; DNA-directed RNA polymerase II subunit I; inositol-3-phosphate synthase; protein transport protein sec22; taurine catabolism dioxygenase TauD/TfdA; ATPase inhibitor, IATP, mitochondria; glycoside hydrolase family 32 protein.

It will be appreciated that the preceding protein classifications correspond to proteins differentially expressed (based on proteomic analysis) in Xanthophyllomyces dendrorhous strain MYM0 relative to the wild-type strain, with reference to Example 3 and Tables 18 and 19.

In some typical embodiments, the isolated protein of this aspect is of a protein classification selected from: ferredoxin/adrenodoxin reductase; cytochrome; ATP synthase; NADH dehydrogenase; fatty acid desaturase; Acyl-CoA-oxidase; pantothenate kinase; polyphosphate multikinase; G protein-coupled receptor; and succinate dehydrogenase. In some typical embodiments, the isolated protein of a classification selected from ferredoxin/adrenodoxin reductase; mitochondrial cytochrome b2; cytochrome b; cytochrome c oxidase subunit 1; ATP synthase subunit 6; NADH dehydrogenase subunit 4; cytochrome c oxidase subunit 2; cytochrome c oxidase subunit 3; NADH dehydrogenase subunit 2; NADH dehydrogenase subunit 5; NADH dehydrogenase subunit 6; cytochrome c oxidase subunit 3; delta 9 fatty acid desaturase; Acyl-CoA-oxidase; pantothenate kinase PanK; geranylgeranyl pyrophosphate synthase; fumarate reductase; sucrose transporter; inositol polyphosphate multikinase, ARGR transcription regulatory complex component; G protein-coupled receptor, rhodopsin-like; succinate dehydrogenase; and ATP synthase subunit mitochondrial.

It will be appreciated that the preceding protein classifications correspond to proteins encoded by one or more nucleotide sequences in which variations considered of particular interest have been identified, with reference to Examples 1 and 2 and Tables 14 and 15.

A related aspect of the invention provides an antibody or antibody fragment that binds, or has been raised against, an isolated protein of the preceding aspect.

As used herein an “antibody” is or comprises an immunoglobulin. The term “immunoglobulin” includes any antigen-binding protein product of a mammalian immunoglobulin gene complex, including immunoglobulin isotypes IgA, IgD, IgM, IgG and IgE and antigen-binding fragments thereof. Included in the term “immunoglobulin” are immunoglobulins that are chimeric or humanised or otherwise comprise altered or variant amino acid residues, sequences and/or glycosylation, whether naturally occurring or produced by human intervention (e.g. by recombinant DNA technology).

Antibody fragments include Fab and Fab′2 fragments, diabodies, triabodies and single chain antibody fragments (e.g. scVs), although without limitation thereto. Typically, an antibody comprises respective light chain and heavy chain variable regions that each comprise CDR 1, 2, and 3 amino acid sequences. A typical antibody fragment comprises at least one light chain variable region CDR and/or at least one heavy chain variable region CDR.

Antibodies and antibody fragments as described herein may be polycolonal or more typically monoclonal. Monoclonal antibodies may be produced using the standard method as for example, described in an article by Kohler & Milstein, 1975, Nature 256, 495, or by more recent modifications thereof as for example described in Chapter 2 of Coligan et al, CURRENT PROTOCOLS IN IMMUNOLOGY, by immortalizing spleen or other antibody producing cells derived from a production species which has been inoculated with an isolated protein or a fragment thereof. It will also be appreciated that antibodies may be produced as recombinant synthetic antibodies or antibody fragments by expressing a nucleic acid encoding the antibody or antibody fragment in an appropriate host cell. Recombinant synthetic antibody or antibody fragment heavy and light chains may be co-expressed from different expression vectors in the same host cell or expressed as a single chain antibody in a host cell. Non-limiting examples of recombinant antibody expression and selection techniques are provided in Chapter 17 of Coligan et al, CURRENT PROTOCOLS IN IMMUNOLOGY and Zuberbuhler et al, 2009, Protein Engineering, Design & Selection 22 169.

In some embodiments, the antibody or antibody fragment is labelled. The label may be selected from a group including a chromogen, a catalyst, biotin, digoxigenin, an enzyme, a fluorophore, a chemiluminescent molecule, a radioisotope, a drug or other chemotherapeutic agent, a magnetic bead and/or a direct visual label.

It will be appreciated that the antibody or antibody fragment of this aspect may be used for the detection and/or purification of an isolated protein disclosed herein.

Another aspect of the invention provides a method of modifying a nucleic acid or protein, including a step of changing one or more nucleotides or amino acids of the nucleic acid or protein, to produce the isolated nucleic acid or the isolated protein of the preceding aspects.

In embodiments, the method of modifying the nucleic acid or protein according to this aspect is a method of mutagenizing the nucleic acid or protein, or a nucleic acid encoding the protein.

In some typical embodiments, the method according to this aspect includes a step of mutagenising a cell or an organism to induce mutations in the genetic material of the cell or organism, to thereby modify the nucleic acid or protein.

The cell may be prokaryotic cell, such as a bacterial cell. In some typical embodiments, the bacterial cell is a Paracoccus cell. The Paracoccus cell may be Paracoccus carotinifaciens.

The cell may be eukaryotic cell, such as a plant cell, an animal cell, an algal cell, and a fungal cell.

In some typical embodiments, the algal cell is a microalgae cell. The microalgae cell may be a Haematococcus cell. In embodiments, the Haematococcus cell is Haematococcus pluvialis.

In some typical embodiments of the fungal cell is a yeast cell. The yeast cell may be a Xanthophyllomyces cell. In some typical embodiments, the Xanthophyllomyces cell is Xanthophyllomyces dendrorhous.

The terms “mutant”, “mutation” and “mutated” are used herein generally to encompass synonymous, non-synonymous, conservative, and nonconservative nucleic acid base pair substitutions, deletions and/or insertions introduced into genetic material. For example, mutations may be introduced into chromosomal DNA and genomic DNA, RNA such as unspliced and spliced mRNA, tRNA and other forms of genetic material as are known in the art.

Mutagenesis of the genetic material of an organism may result in introduction of mutations in one or a plurality of nucleic acid molecules. Genome-wide mutagenesis of organisms is well-known in the art. In alternative embodiments, mutations can be introduced or induced by targeting specific loci or regions. It will be appreciated that gain-of-function and loss-of-function mutations may be achieved as a result of mutagenesis, although without limitation thereto.

Mutations may be induced or introduced using either non-specific methods such as random mutagenesis or alternatively by using specific methods such as targeted mutagenesis. Induced mutations may include single- or multiple-nucleotide substitutions, deletions and/or insertions, either alone or in combination. Mutagenesis methods of the present invention are inclusive of in vitro, in vivo and in situ methodology.

Chemical mutagenesis is a useful method of genome-wide random mutagenesis methods using alkylating agents such as ethylmethanesulfonate (EMS) and dimethyl sulfate (DMS) or other chemical mutagens such as ethidium bromide, formic acid, hydrazine, sodium bisulphite, and diepoxybutane.

Physical mutagenesis using physical mutagens as for example irradiation using ionising radiation (such as β, γ or X-ray radiation), UV irradiation and fast neutron irradiation of cells may also be used for genome-wide random mutagenesis. It will be appreciated by a person skilled in the art that the time and dosage of exposure of the cell or organism, to a mutagen is dependent on the cell, organism, and mutagen that is used and can be readily determined by a skilled person.

Mutations may be introduced into nucleic acids by random or site-directed mutagenesis as are well known in the art. Non-limiting examples of nucleic acid mutagenesis methods are provided in Chapter 8 of CURRENT PROTOCOLS IN MOLECULAR BIOLOGY Eds Ausubel et al., (John Wiley & Sons, Inc. 1995-2008).

Mutagenesis methods can also include incorporation of dNTP analogs into nucleic acids (Zaccolo et al., 1996, J. Mol. Biol. 255 589) and PCR-based random mutagenesis such as described in Stemmer, 1994, Proc. Natl. Acad. Sci. USA 91 10747 or Shafikhani et al., 1997, Biotechniques 23 304. It is further noted that PCR-based random mutagenesis kits have been made commercially available, such as the Diversify™ kit (Clontech).

Mutations produced by a nucleic acid sequence amplification-based technique may be introduced into the genetic material of a cell.

As used herein, a “nucleic acid sequence amplification technique” includes but is not limited to polymerase chain reaction (PCR) as for example described in Chapter 15 of CURRENT PROTOCOLS IN MOLECULAR BIOLOGY Eds. Ausubel et al. (John Wiley & Sons NY USA 1995-2001) strand displacement amplification (SDA); rolling circle replication (RCR) as for example described in International Application WO 92/01813 and International Application WO 97/19193; nucleic acid sequence-based amplification (NASBA) as for example described by Sooknanan et al. 1994, Biotechniques 17 1077; ligase chain reaction (LCR) as for example described in International Application WO89/09385 and Chapter 15 of CURRENT PROTOCOLS IN MOLECULAR BIOLOGY supra; Q-β replicase amplification as for example described by Tyagi et al., 1996, Proc. Natl. Acad. Sci. USA 93 5395 and helicase dependent amplification as for example described in International Publication WO 2004/02025.

Region-specific mutagenesis and directed mutagenesis using PCR may also be employed to construct nucleic acid mutants according to the invention. Oligonucleotide mediated (or site-directed) mutagenesis may also be used. A non-limiting example of oligonucleotide-mediated site-directed mutagenesis procedures to introduce small clusters of point mutations throughout a target region is provided in Ausubel et al., supra. Briefly, mutations are introduced into a sequence by annealing a synthetic oligo nucleotide containing one or more mismatches to the sequence of interest cloned into a single-stranded M13 vector. This template is grown in an Escherichia coli dut- ung- strain, which allows the incorporation of uracil into the template strand. The oligonucleotide is annealed to the template and extended with T4 DNA polymerase to create a double-stranded heteroduplex. Finally, the heteroduplex is introduced into a wild-type E. coli strain, which will prevent replication of the template strand due to the presence of apurinic sites (generated where uracil is incorporated), thereby resulting in plaques containing only mutated DNA. It is also noted that site-directed mutagenesis kits have been made commercially available, such as the QuikChange™ kit (Stratagene).

Alternatively, linker-scanning mutagenesis of DNA may be used to introduce clusters of point mutations throughout a sequence of interest that has been cloned into a plasmid vector. For example, reference may be made to Ausubel et al., supra, (in particular, Chapter 8) which describes a first protocol that uses complementary oligonucleotides and requires a unique restriction site adjacent to the region that is to be mutagenised. A nested series of deletion mutations is first generated in the region. A pair of complementary oligonucleotides is synthesised to fill in the gap in the sequence of interest between the linker at the deletion endpoint and the nearby restriction site. The linker sequence provides the desired clusters of point mutations as it is moved or ‘scanned’ across the region by its position at the varied endpoints of the deletion mutation series.

Mutations may also be induced or introduced by insertion of one or a plurality of nucleotides or base pairs into the genetic material. Transposon and retrotransposon mutagenesis (for example as described in Walbot 2000, Curr Opin Plant Biol 3 103; U.S. Pat. No. 6,720,479; Voytas 1996, Genetics 142 569) are possible methods for insertional mutagenesis. Other methods of insertional mutagenesis include targeted methods such as homologous recombination and site-specific recombination. A non-limiting example of homologous recombination is the T-DNA system (for example as described in Wang et al. 2001, Gene 272 249; and Iida & Terada 2005, Plant Mol. Biol. 59 205). An example of site-specific recombination is the cre-lox recombination system of bacteriophage P1. Chimeric RNA/DNA oligonucleotide-directed gene targeting is also a useful technique for the generation of site-specific point mutations such as deletions, insertions and/or base changes in higher organisms including plants (see for example as described in Iida & Terada, 2005, Plant Mol. Biol. 59 205; and Rice et al., 2000, Plant Physiol, 123 427).

Mutations may also be introduced by deletional mutagenesis of one or a plurality of nucleotides, or a region of a genetic locus. For example, fast neutron deletion mutagenesis can be effective for genome-wide deletional mutagenesis method and utilises fast neutron bombardment to create randomly mutagenised populations, and more particularly knockout mutations such as described Li et al., 2002, Comp. Funct. Genomics 3 158. It will be appreciated that targeted deletional mutagenesis may be achieved by using a variety of other nucleic acid-based mutagenesis methods as herein described, such as, but not limited to oligonucleotide-based mutagenesis.

Targeting Induced Local Lesions in Genomes (‘TILLING’) is particularly amenable for random mutagenesis to generate point mutations in many organisms. TILLING combines traditional chemical mutagenesis following by high-throughput screening for point mutations. Reference is made to McCallum et al., 2000, Nat. Biotechnol. 18 455; Till et al., 2003, Methods Mol. Biol. 236 205; Henikoff et al., 2004, Plant Physiol. 135 630; and Till et al., 2003, Genome Res. 13 524 for non-limiting examples of TILLING methods that may be applicable to the present invention.

In certain embodiments, mutations are introduced into the genetic material of a cell or organism via “genome editing”.

“Genome editing” is a method for mutagenesis in which DNA is inserted, substituted, modified, or deleted from the genetic material of an organism in a targeted manner, typically using engineered nucleases.

Methods for genome editing include ‘zinc finger nuclease’ methods, as described for example by Miller et al., 2007, Nat. Biotech. 25 778; ‘CRISPR Cas’ methods, as described for example by Cong et al., Science 339 819; and ‘TALEN’ methods, as described for example by Bedell et al., Nature 491, 114.

As will be understood by those skilled in the art, genome editing typically comprises the transformation of a cell or tissue with one or more genetic constructs facilitating the expression of:
(i) one or more DNA nucleases; and
(ii) one or more molecules that guide the cleavage of DNA at a targeted region within the genetic material of an organism by said nuclease(s).

Targeted DNA breaks are thereby induced in the genetic material of the organism. These targeted DNA breaks are generally double stranded DNA breaks, although without limitation thereto.

In embodiments of genome editing wherein a zinc finger nuclease method is used, the one or more molecules that guide the cleavage of DNA at a targeted region within the genetic material of an organism by the nuclease(s) are proteins comprising a zinc finger DNA-binding domain. Generally, a plurality of the proteins are fused to the nuclease (s), and the plurality of zinc finger DNA-binding domains of the proteins bind with at least partial specificity to the targeted region, and thereby induce cleavage of the targeted region by the nuclease (s).

In embodiments of genome editing wherein a TALEN method is used, the one or more molecules that guide the cleavage of DNA at a targeted region within the genetic material of an organism by the nuclease(s) are proteins comprising a transcription activator-like effector DNA-binding (‘TALE’) domain. Generally, a plurality of the proteins are fused to the nuclease (s), and the plurality of TALE DNA-binding domains of the proteins bind with at least partial specificity to the targeted region, and thereby induce cleavage of the targeted region by said nuclease(s).

In embodiments of genome editing wherein a CRISPR/Cas method is used, the nuclease is a CRISPR-associated (‘Cas’) nuclease, and the one or more molecules that guide the cleavage of DNA at a targeted region is a “guide” RNA molecule (or ‘gRNA’) with homology to the targeted region. Generally, the gRNA molecule forms a complex with the Cas nuclease and guides binding of the Cas nuclease to the targeted region with at least partial specificity, and thereby induces cleavage of the targeted region by the Cas nuclease.

It will be further understood that targeted DNA breaks induced during genome editing can facilitate non homologous end joining or homology-dependent repair.

“Non-homologous end joining” is a cellular mechanism for DNA break repair wherein cleaved DNA ends are ligated, which is typically ‘error prone’, i.e. introduces nucleotide sequence variation, e.g. insertions or deletions, at the site of the DNA break. DNA breakage followed by error-prone non-homologous end joining induced by genome editing can be used to inactivate targeted regions within the genetic material of organisms (as described for example by Gaj et al., 2013 Trends Microbiol. 31 397).

“Homology-dependent repair” is a cellular mechanism for DNA break repair wherein a nucleic acid possessing homology to the region surrounding a DNA break is used as a template for repair of the DNA break. Genome editing can be used to introduce nucleic acid variants into targeted regions within the genetic material of organisms (as described for example by Gaj et al., 2013 Trends Microbiol 31 397) by inducing DNA breakage followed by homology-dependent repair in the presence of a ‘donor molecule’, wherein said donor molecule comprises homology to the region surrounding the DNA break.

As will be understood by those skilled in the art, genome editing comprising homology-dependent repair can be used for ‘allele replacement’, wherein a nucleic acid sequence of the genetic material of an organism is ‘substituted’, ‘exchanged’, or ‘replaced’ with a variant or variation of the nucleic acid sequence.

In some typical embodiments, the step of mutagenising a cell or organism to induce mutations in the genetic material of the cell or organism to thereby modify the nucleic acid or protein according to this aspect is a step of randomly mutagenising the cell using a chemical and/or physical mutagen.

In some typical embodiments, the chemical mutagen is or includes N-methyl-N′-nitro-N-nitrosoguanidine and/or ethyl methanesulfonate.

In some typical embodiments, the physical mutagen is or includes ultra violet light.

Another aspect of the invention provides a nucleic acid vector or construct comprising the isolated nucleic acid of the invention, and one or more additional nucleotide sequences. Typically, the genetic construct is in the form of, or comprises genetic components of, a plasmid, bacteriophage, cosmid, or yeast or bacterial artificial chromosome, as are well known in the art.

Genetic constructs may be suitable for maintenance and propagation of the isolated nucleic acid in bacteria or yeast or other host cells, for manipulation by recombinant DNA technology and/or expression of the nucleic acid or an encoded protein.

For the purposes of host cell expression, the genetic construct will be an expression construct. Suitably, the expression construct comprises one or more isolated nucleic acids or fragments disclosed herein operably linked to one or more additional sequences in an expression vector.

It will be understood that an “expression vector” may be either a self-replicating extra-chromosomal vector such as a plasmid, or a vector that integrates into a host genome.

By “operably linked” is meant that said additional nucleotide sequence(s) is/are positioned relative to the isolated nucleic acid, typically to initiate, regulate or otherwise control transcription.

In some embodiments, the additional nucleotide sequences are regulatory sequences. Regulatory nucleotide sequences will generally be appropriate for the host cell used for expression. Numerous types of appropriate expression vectors and suitable regulatory sequences are known in the art for a variety of host cells.

The one or more regulatory nucleotide sequences may include, but are not limited to, promoter sequences, leader or signal sequences, ribosomal binding sites, transcriptional start and termination sequences, translational start and termination sequences, and enhancer or activator sequences.

Constitutive or inducible promoters as known in the art may be used. The promoters may be either naturally occurring promoters, or hybrid promoters that combine elements of more than one promoter.

In some embodiments, the additional nucleotide sequence is a selectable marker gene to allow the selection of transformed host cells. Selectable marker genes are well known in the art and will vary with the host cell used.

The expression construct may also include an additional nucleotide sequence encoding a fusion partner (typically provided by the expression vector) so a protein of the invention is expressed as a fusion protein, as hereinbefore described.

By way of example only, an isolated protein of the invention may be produced by a method including the steps of:
(i) preparing an expression construct which comprises an isolated nucleic acid of the invention, operably linked to one or more regulatory nucleotide sequences;
(ii) transfecting or transforming a suitable host cell with the expression construct;
(iii) expressing a recombinant protein in said host cell; and
(iv) isolating the recombinant protein from said host cell.

Suitable host cells for expression may be prokaryotic or eukaryotic. For example, suitable host cells may be mammalian cells, plant cells, yeast cells, insect cells or bacterial cells. In some typical embodiments, the host cell for expression of an isolated protein according to the invention is a bacterial cell. In some typical embodiments, the host cell for expression of an isolated protein according to the invention is an algal cell. In some typical embodiments, the host cell for expression of an isolated protein according to the invention is a yeast cell.

Introduction of genetic constructs into host cells (whether prokaryotic or eukaryotic) is well known in the art, as for example described in CURRENT PROTOCOLS IN MOLECULAR BIOLOGY Eds. Ausubel et al, (John Wiley & Sons, Inc. 1995-2009), in particular Chapters 9 and 16.

Recombinant proteins may be conveniently prepared by a person skilled in the art using standard protocols as for example described in Sambrook, et al, MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al, (John Wiley & Sons, Inc. 1995-2009), in particular Chapters 10 and 16; and CURRENT PROTOCOLS IN PROTEIN SCIENCE Eds. Coligan et al, (John Wiley & Sons, Inc. 1995-2009), in particular Chapters 1, 5 and 6.

It will further be understood that, in some typical embodiments, the vector or construct of this aspect is a gene silencing or gene regulating vector or construct.

It will be appreciated by the skilled person that gene silencing or gene regulating vectors or constructs of the invention are typically adapted to produce non-coding sequences with the capacity to silence or regulate gene expression. Typically, the non-coding sequences are “small RNA” sequences.

As used herein, “small RNA” will be understood to refer to small, non-coding RNA molecules that have the capacity to bind to and regulate the expression, translation and/or replication of other nucleic acid molecules. The skilled person is directed to Ipsaro, J. J., & Joshua-Tor, L., 2015, Nature Struc. & Mol. Biol. 22 20 for summary of small, non-coding RNA molecules.

It will be understood that, as used herein, the term small RNA encompasses all such molecules, regardless of the particular name that may be used in a scientific or research context. By way of non-limiting example, the skilled person will readily appreciate that, as used herein, the term small RNA encompasses small non-coding RNA molecules referred to as “miRNA” and “siRNA”.

It will be further understood that small RNA molecules generally have a high degree of nucleotide sequence identity with a nucleic acid molecule for which they have the capacity to bind to and regulate the expression, translation, and/or replication of However, it will also be understood that a small RNA molecule need not necessarily have 100% identity to such a sequence.

Nucleic acid vectors and constructs for gene silencing or regulation using small RNA molecules are widely available in a range of forms, as is well-known in the art.

In some typical embodiments, the gene silencing or gene regulating vector or construct is adapted for transformation of yeast. For a recent review of small RNA-based gene regulation for strain engineering in yeast, the skilled person is directed to Chen et al. Front. Bioeng. Biotechnol., 2 Jul. 2020 doi.org/10.3389/fbioe.2020.00731.

It will also be understood that, in some typical embodiments, the vector or construct of this aspect is a genome editing construct. Details of genome editing and constructs therefor is provided hereinabove.

In some typical embodiments, the editing construct is adapted for genome editing in yeast. For recent review of genome editing approaches in yeast, the skilled person is directed to Yang, Z., & Blenner, M. (2020). Genome editing systems across yeast species. Current Opinion in Biotechnology, 66, 255-266.

Another aspect of the invention provides a cell comprising the nucleic acid, protein, or vector or construct of the preceding aspects.

In some embodiments, the cell of this aspect is a prokaryotic cell. The prokaryotic cell may be a bacterial cell. The bacterial cell may be Gram-negative or Gram-positive. The bacterial cell may be aerobic, anaerobic, or facultatively anaerobic.

In embodiments, the bacterial cell is of the order Enterobacterales. In embodiments, the bacterial cell is of the family Enterobacteriaceae. In embodiments, the bacterial cell is of the genus Escherichia. In an embodiment, the bacterial cell is Escherichia coli.

In embodiments, the bacterial cell is of the order Caulobacterales. In embodiments, the bacterial cell is of the family Caulobacteraceae. In embodiments, the bacterial cell is of the genus Brevundimonas. In an embodiment, the bacterial cell is Brevundimonas vesicularis.

In embodiments, the bacterial cell is of the order Sphingomonadales. In embodiments, the bacterial cell is of the family Sphingomonadaceae. In embodiments, the bacterial cell is of the genus Sphingomonas. In an embodiment, the bacterial cell is Sphingomonas astaxanthinifaciens.

In embodiments, the bacterial cell is of the order Rhodobacterales. In embodiments, the bacterial cell is of the family Rhodobacteraceae. In embodiments, the bacterial cell is of the genus Paracoccus. In a typical embodiment, the Paracoccus cell is Paracoccus carotinifaciens.

In some typical embodiments, the cell is a eukaryotic cell. The eukaryotic cell may be selected from an animal cell, a plant cell, an algal cell, and a fungal cell.

The animal cell may be a marine animal cell. The animal cell may be a crustacean cell. In embodiments, the animal cell is prawn cell or shrimp cell. In embodiments, the animal cell is a krill cell.

The plant cell may be an angiosperm cell. The plant cell be a monocot or a dicot cell. The plant cell may be an algal cell. The algal cell may be a microalgae cell.

In embodiments, the plant cell is of the order Ranunculales. In embodiments, the plant cell is of the family Ranunculaceae. In a typical embodiment, the plant cell is of the genus Adonis. The plant cell may be of a species selected from Adonis aestivalis, Adonis aleppica, Adonis amurensis, Adonis annua, Adonis bobroviana, Adonis chrysocyathus, Adonis coerulea, Adonis Cyllene, Adonis davidii, Adonis dentata, Adonis distorta, Adonis flammea, Adonis macrocarpa, Adonis nepalensis, Adonis palaestina, Adonis pyrenaica, Adonis ramose, Adonis sibirica, Adonis sutchuenensis, Adonis tianschanica, Adonis vernalis, and Adonis volgensis.

In some embodiments, the algal cell is a microalgae cell. In embodiments, the microalgae cell is of the order Chlamydomonadales. In embodiments, the microalgae cell is of the family Haematococcaceae. In embodiments, the microalgae cell is of the genus Haematococcus. In a typical embodiment, the microalgae cell is Haematococcus pluvialis.

Typically, the cell is a fungal cell. More typically, the cell is a yeast cell.

In embodiments, the yeast cell is of the order Saccharomycetales. In embodiments, the yeast cell is of the family Saccharomycetaceae. In embodiments, the yeast cell is of the genus Saccharomyces. In an embodiment, the yeast cell is Saccharomyces cerevisiae.

In embodiments, the yeast cell is of the family Dipodascaceae. In embodiments, the yeast cell is of the genus Yarrowia. In an embodiment, the yeast cell is Yarrowia lipolytica.

In embodiments, the yeast cell is of the order Cystofilobasidiales. In embodiments, the yeast cell is of the family Cystofilobasidiaceae. In an embodiment, the yeast cell is of the genus Xanthophyllomyces. In a typical embodiment, the cell is Xanthophyllomyces dendrorhous.

A related aspect provides an organism comprising the cell of the preceding aspect. The organism may be any suitable organism inclusive of bacteria, plant, algae, animal, fungi, and yeast organisms.

In some typical embodiments, the organism is an algal strain. More typically, the organism is a yeast strain. Exemplary yeast strains according to this aspect include mutant strains MAMY3, MAMY6, MB18, MB24, MYM0, MYM6, MYM44, and MYM92 as described in Example 1.

It will be appreciated that the cell or organism according to this aspect may be a cell or organism produced according to the mutagenesis approaches for modifying nucleic acid or proteins as hereinabove described.

Another aspect of the invention provides a method of co-cultivating a cell or organism according to the preceding aspect with a further cell or organism. The further cell or organism may be any suitable cell or organism, including prokaryotic or eukaryotic cells and organisms. In some typical embodiments, the further cell or organism is an algal cell or organism. Typically, the algal cell or organism is a microalgae cell or organism. In a typical embodiment, the microalgae cell or organism is Haematococcus pluvialis.

Another aspect of the invention provides a method of producing astaxanthin including a step of expressing the isolated nucleic acid or the isolated protein of the preceding aspects, to thereby produce the astaxanthin.

The expression of the isolated nucleic acid or the isolated protein according to the method of this aspect may be in vitro expression, in vivo expression, or in situ expression. In embodiments, the isolated nucleic acid or isolated protein is expressed in a cell or organism, to thereby produce the astaxanthin. The cell or organism may be a prokaryotic, eukaryotic, animal, plant, algal, microalgal, fungal, or yeast cell or organism as set out in the preceding aspects. Typically, the cell or organism according to the method of this aspect is selected from a bacterial cell or organism, an algal cell or organism, a fungal cell or organism, and a yeast cell or organism.

In some typical embodiments, the method of this aspect includes a step of expressing the isolated nucleic acid or the isolated protein in a bacterial cell, to thereby produce the astaxanthin. In embodiments, the bacterial cell is a Paracoccus cell. The Paracoccus cell may be Paracoccus carotinifaciens.

In some typical embodiments, the method of this aspect includes a step of expressing the isolated nucleic acid or the isolated protein in a microalgae cell, to thereby produce the astaxanthin.

The microalgae cell may be a Haematococcus cell. In embodiments, the Haematococcus cell is Haematococcus pluvialis.

In some typical embodiments, the method of this aspect includes a step of expressing the isolated nucleic acid or the isolated protein in a fungal cell or, more typically, a yeast cell, to thereby produce the astaxanthin. The yeast cell may be a Xanthophyllomyces cell. Typically, the Xanthophyllomyces cell is Xanthophyllomyces dendrorhous.

In some typical embodiments, the astaxanthin is produced according to the method of this aspect at an increased or enhanced level or rate by expression of the isolated nucleic acid or isolated protein, as compared to a level or rate by expression of a corresponding wild-type nucleic acid or protein. Suitably, the corresponding wild-type nucleic acid or protein is a nucleic or protein of wild-type Xanthophyllomyces dendrorhous strain CBS 6938.

In some typical embodiments, the level or rate of expression of astaxanthin is increased or enhanced at least about 5%, 10%, 15%, 20%, 25%, 50%, 75%, 100%, 150%, 200%, 300%, 400%, 500%, 600%, 700%, 800%, 900%, 1000%, 1500%, 2000%, 3000%, 4000%, or 5000%, as compared to the level or rate by expression of the corresponding wild-type nucleic acid or protein.

Another aspect of the invention provides a method of producing astaxanthin including a step of performing metabolism with a cell or organisms expressing an isolated nucleic acid, isolated protein, or vector or construct as hereinabove described, to thereby produce the astaxanthin.

In embodiments, the cell or organism according to this aspect is a prokaryotic, eukaryotic, animal, plant, algal, microalgal, fungal, or yeast cell or organism as hereinabove described. More typically, the cell or organism according to the method of this aspect is selected from a bacterial cell or organism, an algal cell or organism, a fungal cell or organism, and a yeast cell or organism.

In some typical embodiments, the cell is a Paracoccus bacterial cell. More typically, the bacterial cell is a Paracoccus carotinifaciens cell.

In some typical embodiments, the cell is Haematococcus algal cell. More typically, the algal cell is a Haematococcus pluvialis cell.

In some typical embodiments, the yeast cell is a Xanthophyllomyces cell. More typically, the yeast cell is a Xanthophyllomyces dendrorhous.

Typically, the step of performing metabolism with the cell or organism according to this aspect to produce the astaxanthin is a step of performing fermentation with the cell or organism.

Typically, the method of this aspect includes a step of combining the cell or organism with one or more metabolites.

In some typical embodiments, the one or more metabolites comprise a nitrogen source metabolite. Typically, the nitrogen source metabolite is selected from urea, ammonium sulphate, yeast extract, malt extract, bactopeptone, and dried corn steep liquor. More typically, the nitrogen source metabolite is or comprises malt extract.

In some typical embodiments, the one or more metabolites comprise a carbon source metabolite. Typically, the carbon source metabolite is selected from molasses, glucose, glycerol, and sucrose. More typically, the carbon source metabolite is or comprises molasses.

In some typical embodiments, the astaxanthin is produced according to the method of this aspect at an increased or enhanced level or rate by metabolism with the cell expressing the isolated nucleic acid, isolated protein, or vector or construct, as compared to a level or rate by metabolism of a corresponding wild-type cell or organism. In some typical embodiments, the corresponding wild-type cell organism is, or is of, Xanthophyllomyces dendrorhous strain CBS 6938.

In some typical embodiments, the level or rate of expression of astaxanthin is increased or enhanced at least about 5%, 10%, 15%, 20%, 25%, 50%, 75%, 100%, 150%, 200%, 300%, 400%, 500%, 600%, 700%, 800%, 900%, 1000%, 1500%, 2000%, 3000%, 4000%, or 5000%, as compared to the level or rate by metabolism of the corresponding wild-type cell.

In relation to aspects of the invention that can result in increased or enhanced astaxanthin production as hereinabove described, it will be appreciated with reference to Example 1 and Example 2, that increases of astaxanthin production of approaching 50 times (i.e. 5000%) have been achieved using mutant Xanthophyllomyces dendrorhous strains of the invention.

Another aspect of the invention provides a non-astaxanthin by-product or secondary product of the method of the preceding aspect.

The skilled person will readily appreciate that cellular metabolism to produce astaxanthin will typically produce a variety of by-products. In embodiments, the by-product according to this aspect is a by-product of fermentation.

In a typical embodiment, the by-product according to this aspect is an invertase enzyme. The skilled person will appreciate that invertase converts sucrose to glucose and fructose, and is one of the most widely used enzymes in food industry. Invertase is industrially produced by yeast fermentation, such that it could be desirable to produce invertase in conjunction with astaxanthin using the method of the preceding aspect.

Another aspect of the invention provides a formulation comprising the cell (or part thereof), organism (or part thereof), astaxanthin, and/or by-product of the preceding aspects. The formulation will suitably include one or more other components inclusive of buffers, excipients, and diluents, and/or one or more additional active agents.

In some typical embodiments, the formulation of this aspect is a substantially dry formulation, such as a granular or powdered formulation. Typically, the substantially dry formulation comprises astaxanthin.

In some typical embodiments, the formulation of this aspect is a liquid or semi-liquid formulation, such as an aqueous or oil-based formulation. Typically, the liquid or semi-liquid formulation comprises astaxanthin.

In some typical embodiments, the formulation of this aspect is a solid or semi-solid formulation, such as an oleoresin or encapsulated oleoresin formulation. Typically, the solid or semi-solid formulation comprises astaxanthin.

In some typical embodiments, the formulation of this aspect, such as the substantially dry formulation, liquid or semi-liquid formulation, or solid or semi-solid formulation, comprises cell wall derivatives of the cell (such as the plant cell or more typically the yeast cell) of the cell or organism according to the invention as herein described.

The formulation of this aspect may be adapted for administration to or consumption by an animal.

In some typical embodiments, the formulation is adapted for administration to or consumption by a farmed animal. The farmed animal may be an aquaculture animal, typically a seafood aquaculture animal.

In some typical embodiments, the formulation is adapted for administration to or consumption by a pet animal, a domestic animal, or a companion animal. The pet animal, domestic animal, or companion animal may be a feline animal (e.g. cat), a canine animal (e.g. dog), or an equine animal (e.g. horse). The formulation may be a pet food formulation or the like.

In some typical embodiments, the formulation is adapted for administration to or consumption by a human. The formulation adapted for consumption by a human may comprise protein. In some typical embodiments, the formulation is an astaxanthin-supplemented protein formulation such as a protein bar, protein drink, or protein powder, or the like.

In some typical embodiments, the formulation of this aspect comprises beta-glucans.

In some typical embodiments, the formulation of this aspect comprises polyphenols.

Another aspect of the invention provides a method of supplementing an animal with the astaxanthin or formulation according to the preceding aspects.

In some typical embodiments of this aspect, the animal supplemented according to the method of this aspect is a farmed animal. Typically, the farmed animal is an aquaculture animal. Typically, the aquaculture animal is a crustacean or a fish. In some typical embodiments, the crustacean is selected from shrimp, krill, crab, and crayfish. In some typical embodiments, the fish is selected from salmon and trout. A related aspect provides a farmed animal product produced by or from a farmed animal, such as a seafood animal, supplemented according to the method of this aspect.

In some typical embodiments, the animal supplemented according to the method of this aspect is a pet animal, a domestic animal, or a companion animal. Typically, the pet animal, domestic animal, or companion animal is selected from a feline animal (e.g. domestic cat), a canine animal (e.g. domestic dog), and an equine animal (e.g. domestic horse).

In some typical embodiments, the supplemented animal is a human.

Another aspect of the invention provides a method of treating or preventing a disease or disorder in a subject, including a step of administering the astaxanthin or formulation according to the preceding aspects to the subject. Suitably, the subject is an animal subject. Typically, the animal subject is a human.

In some typical embodiments, the disease or disorder treated according to the method of this aspect is a wound. The method according to this aspect may be a method of treating, alleviating symptoms or, or controlling infection in a wound. The administration may be topical administration to the wound and/or oral administration to the subject.

A related aspect provides use of the astaxanthin, by-product, or secondary product according to the preceding aspect in the manufacture of a formulation, composition, or medicament for the treatment of a disease or disorder in a subject.

### EXAMPLES

### Example 1: Enhanced Astaxanthin Production in Mutant Xanthophyllomyces dendrorhous Strains

Astaxanthin (AX) is a potent antioxidant with increasing biotechnological and commercial potential as a feed supplement. AX is also known for giving salmonids and crustaceans their characteristic pink colour. The red yeast Xanthophyllomyces dendrorhous (previously known as Phaffia rhodozyma) naturally produces AX as its main fermentation product but wild-type strains and those previously generated through classical random mutagenesis produce relatively low yields of AX, such that existing strains do not meet desired commercial requirements.

This example describes X. dendrorhous CBS 6938 mutant strains generated through chemical and ultraviolet radiation mutagenesis in combination with screening, the strains exhibiting comparatively high AX production. Additionally, this example describes enhancement of mutant X. dendrorhous strain AX production using culture media optimization and fed-batch culture kinetic modelling. Under optimised conditions, an approximately 50-fold increase in AX production as compared to the wild-type strain has been achieved, with a total biomass of around 100 gDCW/L and a carotenoid production of 1 g/L.

This example further describes whole genome sequencing of eight X. dendrorhous mutant strains showing substantially increased AX production to identify genomic changes. Genomic variant analyses found 368 conserved mutations across the selected strains with notable mutations including those identified in regulators and catalysts of AX precursors in the mevalonate pathway, the electron transport chain, oxidative stress mechanisms, and carotenogenesis.

### Materials and Methods

### Strains, Media, and Growth Conditions

The wild-type strain of X. dendrorhous CBS 6938 was obtained from the CBS Fungal Biodiversity Centre culture collection. The strain was kept on agar plates at 4° C. for short term storage of up to one month or glycerol stock (glycerol 20%) at −80° C. for long term storage. YM media containing (g/L): yeast extract (3), malt extract (3), bactopeptone (5), and sucrose (20) as a carbon source was used for routine liquid culture and inoculum preparation. Agar (15 g/L) was supplemented to prepare YM agar plates. When specified, YM media was buffered with potassium hydrogen phthalate (20 g/L). For flask fermentations, 50 mL of liquid YM media in a 250 mL shake-flask was cultured on a rotary shaker at 250 rpm and 20° C. Flasks for fermentation were inoculated with a pre-culture growing at the mid-exponential phase by using 10% of inoculum at A600.

### Analytic Techniques

### Optical Density (A600)

The absorbance of cultures with X. dendrorhous was measured at 600 nm in a spectrophotometer. The sample was diluted within a range of 0.1-1 absorbance units.

### Dried Cell Weight

One to two millilitres of cells were vacuum filtered using a Millipore HAWP04700 filter (pore size of 0.45 μm and diameter of 47 mm) with the filter humidified with distilled water before filtrating the cells. The filter with filtered cells was then dried in an oven at 80° C. for 48 h and weighed. The difference between the weight of the filter with and without cells, and the volume taken as a sample, were used to calculate the dried cell weight, represented as gDCW/L.

### Total Carotenoids

The DMSO technique was used to determine the total carotenoids of samples (Sedmak et al., 1990). For this, 5-50 pL of sample was placed into a 2 mL Eppendorf tube and centrifuged at 14,000 rpm for 1 min, in which the supernatant was discarded and the pellet kept. The cells were then washed two times with distilled water. Next, 0.5 mL of heated DMSO at 55° C. was added to the pellet and vortexed for 30 s. To ensure total disruption, the cells were incubated for an additional five minutes at 55° C. and vortexed again for 30 sec. Next, 0.1 mL of 0.01 M sodium phosphate at pH 7 and 1 mL of 1:1 hexane:ethyl acetate was added, followed by vortexing for 30 s. The tube was then centrifuged at 14,000 rpm for 1 min to separate the solution into two phases. Finally, 0.7 mL of the organic phase (the phase on the top with the pigments) was transferred into a 700 pL Quartz Cuvette to measure absorbance at 480 nm. The total carotenoids were calculated with the following equation:

$$Total Carotenoids , \frac{u g}{L} = \frac{[ A 480 ] [ V 1 ] [ 1 E 1 {0}^{7} ]}{[ E ] [ L ] [ V 2 ]}$$

A480: Absorbance at 480 nm; V1: Volume of the organic phase, mL; E: Extinction coefficient of 2150; L: Length of the Quartz Cuvette (generally 1 cm); V2: Volume of the sample, mL.

### Hplc Analyses for Astaxanthin and Other Carotenoids

The samples were obtained using the DMSO extraction method for total carotenoid analyses. Samples and standards were diluted in hexane:ethyl acetate 1:1 (v/v) before injection of 30 pL. The column was a Luna® 3 pm Silica (2) 100 A, LC Column 150×4.6 mm (Phenomenex Cat. No. 00F-4162-E0). The column was equipped with a security guard cartridge holder (Phenomenex Cat. No. KJO-4282) and a security guard cartridge Silica 4×3 mm ID (Phenomenex Cat. No. AJO-4348) so as to extend lifespan. The mobile phase was a mixture of hexane:acetone (82:18, v/v) which was injected at a flow rate of 1.2 mL/min. The column temperature was ambient and carotenoids were detected in a UV/VIS detector at 474 nm. Standards of astaxanthin, p-Carotene, canthaxanthin, zeaxanthin, 9-cis-AX, 13-cis-AX were used to identify carotenoids in the sample. When required, the column was cleaned with 10 volumes each of hexane, methylene chloride, isopropanol, methylene chloride, and hexane:acetone (82:18, v/v). For water removal (when needed), the column was flushed with 60 mL of 2.5% of 2,2-dimethoxy propane, and 2.5% of glacial acetic in hexane. The column was stored in hexane or isopropanol.

### Carbon Source

### Glucose

The DNS method was used to measure the concentration of reducing sugars (Miller, 1959). The DNS solution consisted of (g/100 mL): DNS (1), NaOH (1.6), KOH (2.24), Na K Tartrate (30) to 100 mL of distilled water. The technique was adapted so as to be undertaken in microplates of 96-wells where 10 pL of each sample was placed into a 200 pL well of a PCR plate. Then 70 pL of water was added to each well. The blank used 80 pL of water. Next, 120 pL of the DNS solution was added to each well. This solution was centrifuged for 5 min at 4000 g and heated in a thermocycler for 5 min at 100° C. Finally, 170 pL was transferred into a 96-well plate to measure absorbance at 540 nm in a microplate reader. A linear growth curve of glucose (in a range of 0.1 to 10 g/L) was created and used to calculate the concentration of glucose in the sample in g/L.

### Sucrose

Sucrose was measured using the DNS technique, as for glucose. However, the sucrose was firstly inverted using an acid treatment where 10 pL of the sample was treated with 2 pL of HCl 37% at 90° C. for 5 min. To neutralise the solution, 5 pL of NaOH 10 M was added. After the inversion, the technique was performed as for glucose. The concentration of sucrose was calculated using a linear curve in the linear range of 0.1 to 10 g/L.

### Mutagenic and Screening Methods

### Mutagenic Technique Using N-Methyl-N′-Nitro-N-Nitrosoguanidine (NTG)

Cells growing at the mid-exponential phase were washed twice with citrate buffer 0.1 M at pH 5.5. The cells were then treated with NTG at 0.1 g/L (dissolved in citrate buffer) for 30 min. These treated cells were then washed twice with a phosphate buffer 0.1 M at pH 7. Before plating on YM agar plates, treated cells were incubated on YM media on a rotary incubator at 20° C. and 250 rpm for 3 h. The plated cells were incubated at 20° C. for seven days. Cells with an increased red-colour were selected and the NTG treatment repeated. This protocol was performed until any increasing concentration of the red colour was no longer observed.

### Mutagenic Technique Using UV-Light

Cells growing at the mid-exponential phase were washed twice with a phosphate buffer 0.1 M at pH 7 and centrifuged at 4000 rpm for 5 min. Cells adjusted to an A₆₀₀ of 0.3, and 5 mL were placed in a plate and treated with the UV-light source of a biosafety cabinet for 10 min.

### Mutagenic Technique Using Ethyl Methyl Sulfonate (EMS)

Cells growing at the mid-exponential phase were washed twice with a phosphate buffer 0.1 M at pH 7. Cells were centrifuged at 4000 rpm for 5 min. Cells were adjusted to an A₆₀₀ of 0.3, and 5 mL were resuspended in the same buffer containing 4% EMS and treated for 2 h. Finally, treated cells were washed twice with the phosphate buffer and subjected to further screening analyses.

Screening Methods with Selective Pressure

Cells treated with the different mutagenic agents were incubated for 3 h in YM media at 20° C. and plated YM agar plates supplemented with 1 mM β-ionone (Bon et al., 1997), 75 pM diphenylamine (Chumpolkulwong et al., 1997), or 50 pM of antimycin A (Sigma Cat. No. A8674) (An et al., 1989). To perform screening with fluorescence activated cell sorting (FACS), treated cells were grown in a buffered YM media, stressed with 20 mM H₂O₂ after 1 day of incubation, supplemented with 20 g/L of sucrose after 3 days of incubation and allowed to grow for an additional 3 days.

Screening and selection of putatively superior strains using FACS was performed as described in Ukibe et al., 2008. Initially, the BD FACS Aria II flow cytometer, equipped with an ion laser emitting at 488 nm, was used to select putatively superior strains. Before analyses, cells were washed twice with a 10 mM potassium phosphate buffer (pH 7.4) and filtered through a 40 mm nylon mesh and placed into a 5 mL polystyrene 12×75 mm tube (BD Cat. No. 352063). Florescence emissions were measured in two channels at wavelengths of 490-550 nm and 665-685 nm. In further screenings, the cells were screened in a BD FACS ARIA III Cell Sorter using a yellow-green laser excited to 561 nm and a blue laser excited to 488 nm. The forward scatter signal (FS), side scatter signal (SC), and fluorescence intensities were measured simultaneously. Fluorescence emission was measured at 670/14 nm and 530/30 and cells with the highest fluorescence were collected in YM sterile medium. Sorted cells were then plated in YM plates and incubated for 7 days at 20° C. In all strategies, colonies with an increased red colour were selected for further analyses.

### High Throughput Screening

Selected colonies were cultivated in 24 deep well plates (Axygen Cat. No. AX-P-DW-10ML-24-C) with 3 mL of YM buffered culture media in each well. The plates were covered with breathable paper (Axygen Cat. No. AX-BF-400-S-1) to allow the exchange of gasses. The plates were incubated in a shaker incubator at 250 rpm and 20° C. for 6 days. The cells were stressed with 20 mM of H₂O₂ at day 1 of incubation and allowed to grow for an additional 2 days before being supplemented with 30 g/L of sucrose and allowed to grow for an additional 3 days.

### Culture Media Optimization

Nutritional requirements were studied in three steps. First, single factor designs were used to test the effect of different nitrogen and carbon sources. A response surface was then used to optimise the significant media components in a screening factorial design. Finally, a feeding profile was designed in a fed-batch culture to maximise AX production.

### Single Factor Design Experiments

The media composition to test nitrogen sources was made of (g/L): magnesium sulphate (1.5), monobasic potassium phosphate (1.5), and sucrose (20). Potassium hydrogen phthalate was used as a buffer (20 g/L). The pH was adjusted to 5.5 with NaOH 2 M. The nitrogen sources tested were (g/L): yeast extract (5), malt extract (5), bactopeptone (5), dried corn steep liquor (5), urea (2.14) and ammonium sulphate (4.71). The media to test different carbon sources was made of (g/L): ammonium sulphate (5), magnesium sulphate (1.5), monobasic potassium phosphate (1.5), yeast extract (3), dried corn steep liquor (5), and malt extract (3). Potassium hydrogen phthalate was used as a buffer (20 g/L). The carbon sources tested were sucrose, glucose, molasses, and glycerol.

### Statistical Experimental Design and Surface Response

An initial screening test was conducted with seven components of the culture media using a factorial design 2^{k-p} (k=factors=7; p=fractionation=4). The basal media to perform the design was made of (g/L): magnesium sulphate (1.5), calcium chloride (0.8), iron sulphate heptahydrate (0.019), and potassium hydrogen phthalate as a buffer (20). The media was supplemented with trace salts (mg/L): citric acid (15), ZnSO₄·7H₂O (5); CuSO₄·5H₂O (0.75); MnSO₄ (0.60); H₃BO₃ (0.60); Na₂MoO₄-2H₂O (0.60); KI (0.15), and vitamins (mg/L): vitamin B3 (Niacin) (3); vitamin B5 (Pantothenic acid) (4.5); vitamin B1 (Thiamine) (3); vitamin B6 (Pyridoxine) (0.3); vitamin B7 (Biotin) (0.18); p-aminobenzoic acid (1.8). The pH was adjusted to 5.5 with sodium hydroxide 2 M. The factors tested were bactopeptone (Low Level 1 g/L; High Level 3 g/L), malt extract (Low Level 1 g/L; High Level 3 g/L), yeast extract (Low Level 3 g/L; High Level 5 g/L), dried corn steep liquor (Low Level 2 g/L; High Level 5 g/L), potassium phosphate monobasic (KH₂PO₄) (Low Level 1 g/L; High Level 2 g/L), Sucrose (Low Level 20 g/L; High Level 30 g/L), or a vitamin cocktail (Low Level 1X; High Level 2X). Further optimization was performed using a Central Composite Design and Response Surface Methodology.

### Model Development for Fed-Batch Design

The model was first developed as a batch culture and then extrapolated to a fed-batch system. The parameters of the batch model were obtained from fermentations on flask culture using 10, 15, and 25 g/L in an optimised culture media. The model was developed on the following assumptions:
1. Sucrose is the only limiting carbon source;
2. There is no nitrogen limitation;
3. The pH is known and controlled throughout the fermentation at pH=5.5.

### Batch Model

The differential mass balance equations (1) to (6) describe the dynamics of AX production in batch fermentation as follows:

$$\frac{d X}{d t} = uX ( biomass ) \qquad ( 1 )$$

$$\frac{d S}{d t} = - qsX ( sucrose ) \qquad ( 2 )$$

$$\frac{d P}{d t} = qpX ( carotenoids ) \qquad ( 3 )$$

$$u = u max ( \frac{S}{K s + S} ) {( \frac{1 - P}{K p} )}^{r h o} ( specific growth rate ) \qquad ( 4 )$$

$$qs = ( u / Yxs ) + ms ( Specific substrate rate ) \qquad ( 5 )$$

$$qp = ( α * u ) + β ( Specific production rate ) \qquad ( 6 )$$

Eq. (1) represents the growth rate and Eq. (4) its specific rate (μ). Eq. (2) represents the consumption rate of sucrose and Eq. (5) its specific rate. Eq. (3) represents the carotenoid expression and Eq. (6) its specific production rate that considers growth associated and non-associated production.

### Fed-Batch Model

The batch model was extrapolated to a fed-batch culture to design the feeding profile.

The fed-batch model is represented as equations (7) to (11) whereby a differential equation to represent volume (V) used to calculate the factor dilution (D) were added to the equations (1) to (3).

$$\frac{d X}{d t} = u X - DX ( biomass ) \qquad ( 7 )$$

$$\frac{d S}{d t} = - qsX + D ( S o - S ) ( sucrose ) \qquad ( 8 )$$

$$\frac{d P}{d t} = q p X - DP ( carotenoids ) \qquad ( 9 )$$

$$\frac{d V}{d t} = F ( volume ) \qquad ( 10 )$$

$$D = F / V ( factor dilution ) \qquad ( 11 )$$

In equation (8), So represents sugar concentration in the feeding solution. Equations (12) and (13) were used to design an exponential feeding profile:

$$F exp = F {o ( e )}^{u t} ( exponential feeding rate ) \qquad ( 12 )$$

$$Fo = ( \frac{u}{Yxs} ) ( X o ) ( Vo ) ( initial feeding rate ) \qquad ( 13 )$$

Where Xo and Vo represent the initial biomass and volume, respectively.

### Reliability of the Model

The coefficient of determination (R²) was used to determine the reliability of the model. The R² was calculated as follows:

$${R}^{2} = \frac{1}{m} {\sum }_{j = 1}^{m} ( 1 - \frac{SSE}{SST} )$$

$$SSE = {\sum }_{i = 1}^{n} {Δ}_{i}^{2}$$

$$SST = {\sum }_{i = 1}^{n} {( {y}_{i} - \overline{y} )}^{2}$$

$$\overline{y} = \frac{1}{n} {\sum }_{i = 1}^{n} {y}_{i}$$

### Parameter Estimation of the Model

The model parameters were obtained from batch fermentations at different initial carbon sources. The package SBPDgui of the System Biology Toolbox 2 (SBTOOLBOX2) (Schmidt and Jirstrand, 2006) was used to determine the model parameters.

### Instrumented Fermenters

Laboratory optimization of AX production in X. dendrorhous was performed in a 2 L Biostat A fermenter. The fermenter was configured with 2 six-blade Rushton impellers with a diameter of 5 cm, three baffles, one ring sparger, and ports for acid, base, antifoam, feeding solution, and sampling. The fermenters were equipped with probes and controllers of pH, dO₂, temperature, and antifoam to measure and control these parameters, respectively. Optimal fermenter conditions were as follows: the fermenter was inoculated using a 10% inoculum culture at an A₆₀₀ from 5 to 10 (cells growing at the mid-exponential phase). The pH was maintained at 5.5 using 12.5% of Ammonium Hydroxide or 2 M of sulphuric acid. The foam was controlled by adding Antifoam C (Sigma Cat. No. A8011). The temperature was controlled at 20° C. using a heater jacket or chiller. The dissolved oxygen was controlled at 70% of air saturation by using cascade changes in agitation (400-1200 rpm), air flow rate (0.4-5 VVM), or pure oxygen flow rate (0-0.5 VVM).

### Culture Medium for Fed-Batch Fermentation

The following media was used for high cell densities in fed-batch fermentations (BYM). The BYM media was made of (g/L): yeast extract (5), malt extract (5), monobasic potassium phosphate (5), magnesium sulphate (1.5), ammonium sulphate (4), FeSO₄·7H₂O (0.10), CaCl₂) (0.4), sucrose (20). The media was supplemented with trace salts (mg/L): citric acid (225); ZnSO₄·7H₂O (75); CuSO₄·5H₂O (11.25); MnSO₄ (9); H₃BO₃ (9); Na₂MoO₄·2H₂O (9); KI (2.25), and vitamins (mg/L): vitamin B3 (Niacin) (18.99); vitamin B5 (Pantothenic acid) (28.48); vitamin B1 (Thiamine) (18.99); vitamin B6 (Pyridoxine) (1.89); vitamin B7 (Biotin) (1.13); p-aminobenzoic acid (11.39).

### Feeding Solution and Feeding Strategy

The BYM 2X at 500 g/L of sucrose was used for the growth phase (0-3.5 days) of the fed-batch culture. The BYM 2X was made of (g/L): yeast extract (10), malt extract (10), monobasic potassium phosphate (10), FeSO₄·7H₂O (0.20), CaCl₂) (0.8), and sucrose (500). Carbon source and culture medium components were sterilised separately at 121° C. for 15 min. The media was supplemented with trace salts (mg/L): citric acid (345); ZnSO₄·7H₂O (5); CuSO₄·5H₂O (17.25); MnSO₄ (13.8); H₃BO₃ (13.8); Na₂MoO₄·2H₂O (13.8); KI (3.45), and vitamins (mg/L): vitamin B3 (Niacin) (30); vitamin B5 (Pantothenic acid) (45); vitamin B1 (Thiamine) (30); vitamin B6 (Pyridoxine) (3); vitamin B7 (Biotin) (1.8); p-aminobenzoic acid (18). The pH was adjusted to 5.5 with sodium hydroxide 2 M. From the 3.5 to 7 days of cultivation, the feeding solution consisted of sucrose at 800 g/L. The feeding strategy was designed using the kinetic model for a fed-batch culture. The feeding profile consisted of batch—fed-batch (exponential feed rate at 0.08 h⁻¹)—fed-batch (constant feeding rate at 4.16 mL/h)—batch.

### Genome Sequencing and Bioinformatics Analyses

### DNA-Sequencing

DNA was extracted using the YeaStar Genomic DNA Kit (Zymoresearch Cat. No. D2002) and quantified using the Nanodrop 1000 (Thermo Scientific) and Qubit dsDNA BR assay kit (Life Technologies Cat. No. Q32850). The DNA quality was determined by running a 1% agarose gel with a DNA gel stain SYBR safe (Life Technologies Cat. No. S33102). The gel was visualised using a ChemiDoc MP system (Bio-Rad). The Illumina platform was used to sequence the genomes of nine strains (wild-type X. dendrorhous CBS 6938 and eight mutant strains). Sequencing was performed using MiSeq V3 600 Cycle 300 PE (Illumina Cat. No. MS-102-3003). Libraries were prepared using the Illumina Nextera XT library preparation kit (Illumina Cat. No. FC-121-2003).

### Bioinformatics

Sequenced genomes were analysed using the following bioinformatics tools. Firstly, FastQC was used to evaluate the quality of the Illumina reads (Andrews, 2010). Trimmomatic was then used to remove poor quality reads (Bolger et al., 2014). Assembly of reads was performed using a reference genome. For this step, Bowtie2 was used to align the reads against the publicly available genome X. dendrorhous CBS 6938 (accession ids in the European Nucleotide Archive: LN483084-LN483350) (Langmead and Salzberg, 2012; Sharma et al., 2015). Then, the Velvet genome assembly algorithm was used to assemble the reads (Zerbino, 2010). To objectively determine genome similarity, the genome-to-genome distance calculator (GGDC 2.0) was used to calculate the genome distance between two genomes (Auch et al., 2010). The TMHMM server 2.0 was used to predict transmembrane helix proteins (Krogh et al., 2001). Variant analyses was performed with GATK by using the recommended best practices for a non-model organism (Auwera et al., 2013; Kryvokhyzha, 2016). Here, BWA-MEM was used to align the Illumina reads against the published genome X. dendrorhous CBS 6938 (Li and Durbin, 2010; Sharma et al., 2015). In further filter analyses, the detected variants, namely SNPs and INDELs, were filtered out if the number of reads harbouring the mutation were below the total reads. BCFtools and Samtools were used to compare and manipulate the VCF files resulting from the variant analyses (Li et al., 2009). SnpEff was used to annotate the variants and SnpSift to filter them (Cingolani et al., 2012). IGV viewer was used for visualization (Thorvaldsdottir et al., 2013).

### Results

Mutant Strains with Enhanced Carotenoid Production

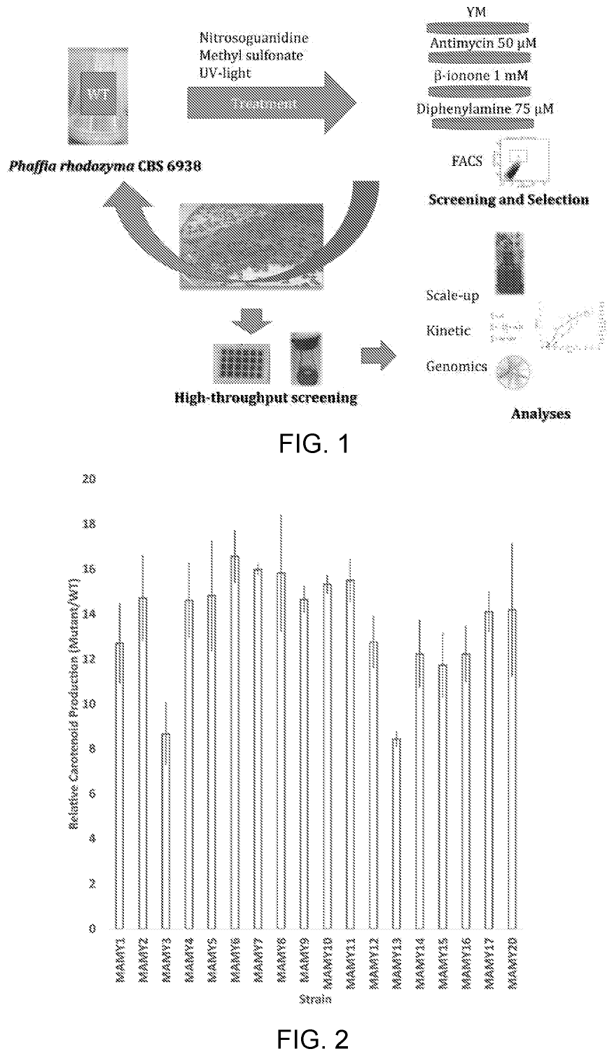

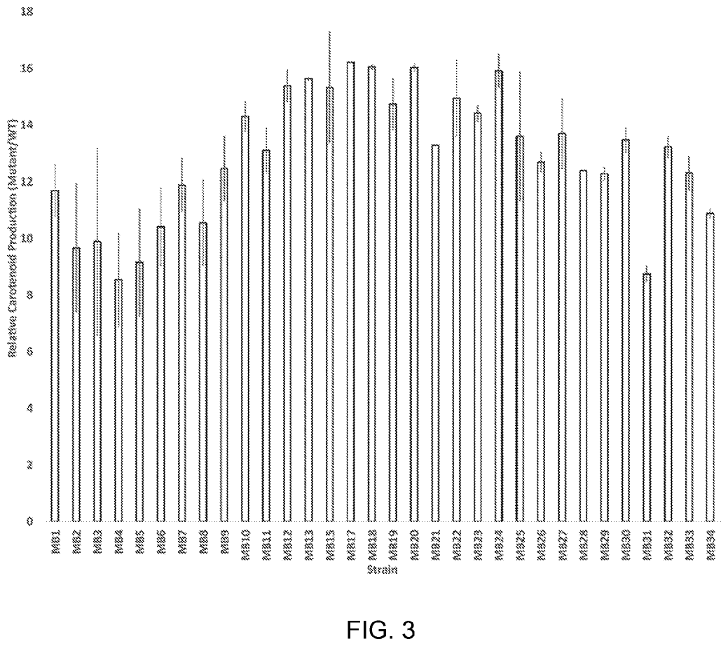

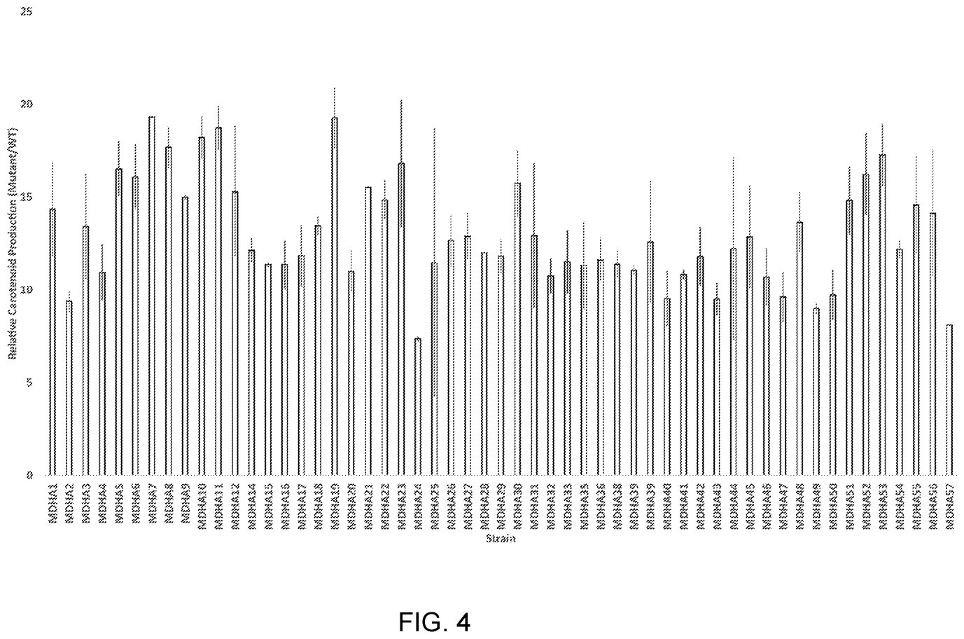

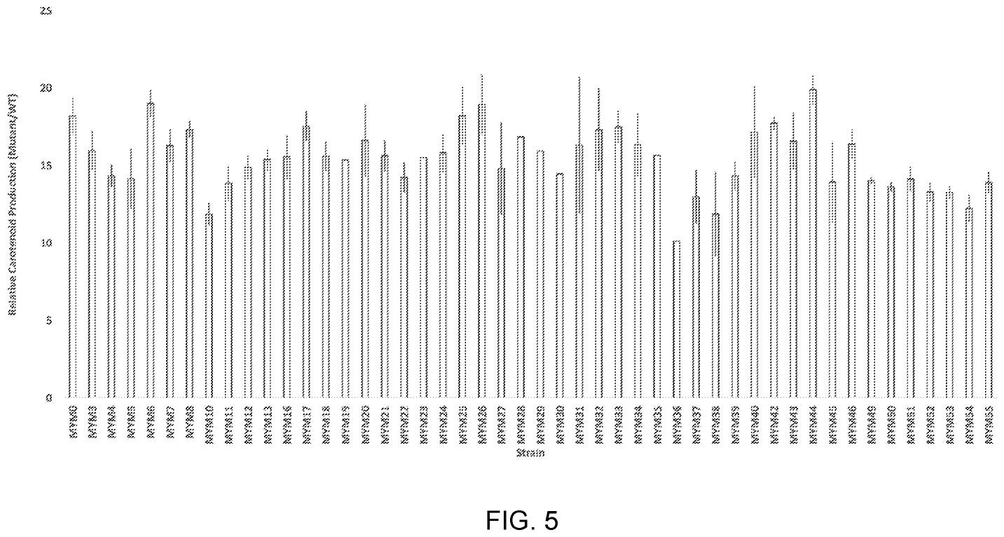

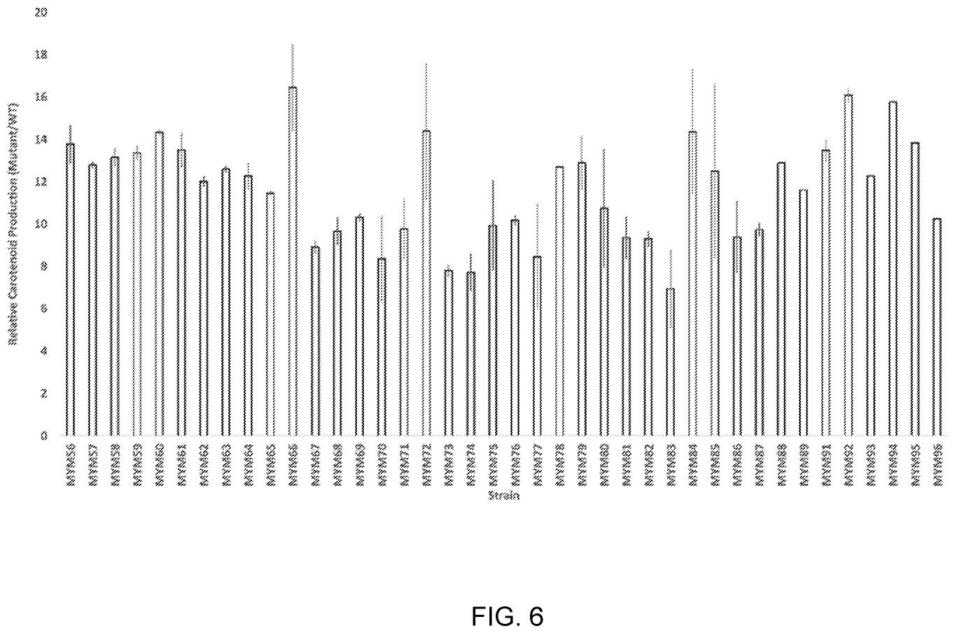

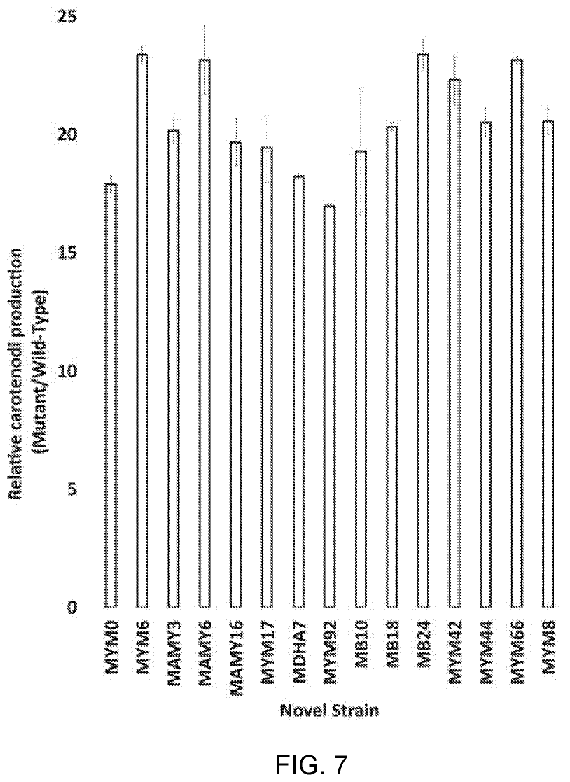

The wild-type X. dendrorhous CBS 6938 was submitted to recursive NTG mutagenic cycles and screening in YM plates until a visible plateau of pink-colour improvement was achieved. After four mutagenic cycles, a pool of the best strains was selected to perform further mutagenic cycles using either UV-light, EMS, or NTG, with different screening methods including fluorescence activated cell sorting, antimycin, (3-ionone, or diphenylamine (FIG. 1). From 187 selected strains, and after screening in deep-well liquid culture plates (see FIGS. 2-6), 15 strains were selected for testing in shake flask cultures: MYM0, MYM6, MAMY3, MAMY16, MAMY17, MDHA7, MDHA19, MYM19, MYM92, MB10, MB18, MB24, MYM42, MYM44, MYM66, and MYM8. FIG. 7 demonstrates that carotenoid production was increased 18 to 23-fold in these strains as compared to the wild-type strain.

### Single Factor Design Experiments

Different organic and inorganic nitrogen sources were evaluated for growth and production of AX by mutant X. dendrorhous strain MYM0 (Table 2). Single factor statistical analyses gave significant results (p<0.05) for the response variables of growth (A₆₀₀) and production (total carotenoids and intracellular carotenoid content (Ypx)). Here, except for malt extract, all nitrogen sources tested had similar growth performances (A₆₀₀) with growth from 18.60 to 21.76. Malt extract had the highest intracellular carotenoid content (Ypx) with 3.94, 1.85, 1.09, 1.19, and 1.40-fold improvements compared to urea, ammonium sulphate, yeast extract, bactopeptone, and dried corn steep liquor, respectively. The best performing nitrogen sources in terms of carotenoid production were yeast extract and bactopeptone, followed by ammonium sulphate, dried corn steep liquor, malt extract, and urea. Table 2 shows that urea led to the lowest specific carotenoid production, suggesting that it may be a sub-optimal nitrogen source for carotenoid production in this strain. The carbon sources molasses, glucose, glycerol, and sucrose were also tested for growth and carotenoid production. MYM0 was able to grow and produce carotenoids in all carbon sources tested (Table 3). Here, the observed growth (A₆₀₀) was from 19.79 to 27.89, specific production from 3132 μg/gDCW to 4778 μg/gDCW, and carotenoid production from 36,047 to 53,395 μg/L. Except for molasses, all carbon sources had a similar growth performance. Glycerol and molasses led to similar levels of catotenoid production, with an improvement of 1.52-fold compared to glucose and sucrose. Of note is that the lag phase for growing on glycerol was extended for three days compared to six hours for the other carbon sources.

### Factorial Design and Surface Response

Initial screening was conducted with seven components of the culture media by using a factorial design of 2^{k-p} (k=7; p=4). Tables 5, 6, and 7 show results for the analyses of variance for growth, carotenoid production, and specific yield, respectively. The significant factors were bactopeptone, malt extract, yeast extract, sucrose, and dried corn steep liquor (p<0.05). These factors were further used to perform a Central Composite Design (for the experiments) and Response Surface Methodology for identifying the optimal levels). The effects of malt extract, yeast extract, Sucrose, and dried corn steep liquor were studied at five experimental levels (−1.6818, −1, 0, 1, 1.6818) for growth and production. Sucrose, malt extract, and yeast extract stimulated production. In contrast, dried corn steep liquor was observed to be detrimental for carotenoid production. As a result, this media component was removed from further experiments. Experimental results were fitted to a predictive quadratic model and identify the conditions for maximal carotenoid production. Table 8 demonstrates the model was able to predict production with a Coefficient of Variance within 5%.

### Fed-Batch Fermentation

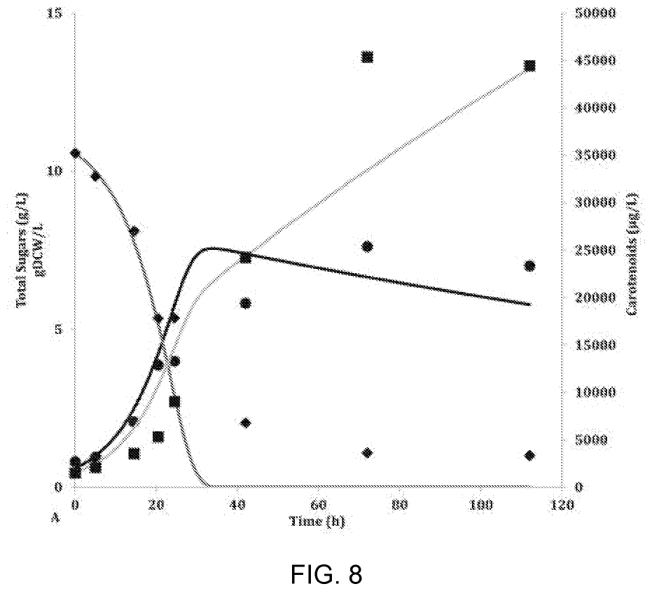

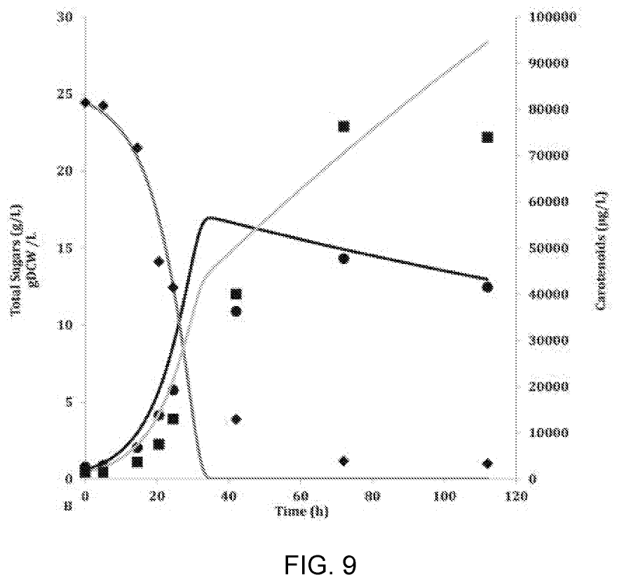

After culture media optimization, production was scaled-up to instrumented fermenters using a non-structure model. This model was validated using kinetic data in a batch mode at 10 g/L and 25 g/L of initial sucrose concentration (FIG. 8 and FIG. 9) where the coefficients of determination were 0.95 and 0.93, respectively. The model was then extrapolated to a fed-batch culture and used to design feeding strategy. Growth and production was divided into four phases: Batch—Exponential Feeding Rate—Constant Feeding Rate—Batch. After seven days of fermentation, the MYM0 strain was able to grow to 117±4.24 gDCW/L and produce 967±15 mg/L of carotenoids, wherein the carotenoid composition was 75% for AX, 5.5% for β-carotene, 4.5% for cantaxanthin, and 15% for other carotenoids. There was no detectable zeaxanthin, 9-cis-AX, or 13-cis-AX in the carotenoid fraction. The coefficient of determination was 0.94 suggesting a reliable model to predict growth and production in P. rhodozyma.

### Bioinformatics Analyses

From the strains screened for production (FIG. 2), X. dendrorhous MYM6 (coded for sequencing as MYM6_Y2), MYM0 (coded as MYM0_Y11), MYM44 (coded as MYM44_Y14), MB18 (coded as MB18_Y15), MAMY3 (coded as MAMY3_Y16), MB24 (coded as MB24_Y17), MYM92 (coded as MYM92_Y4), and MAMY6 (coded as MAMY6_Y12) were selected for whole genome sequencing using the Illumina platform. The wild-type X. dendrorhous CBS 6938 was re-sequenced as a control. Genome assembly was performed with Velvet, including adapter removal, trimming, and filtering of poor-quality reads. The contigs were then filtered to leave those with a length>300 bp, and coverage above 10X. The genome size, number of contigs, N50, and GC content is presented in Table 4. The genome sizes were from 18.55 Mb to 19.11 Mb. The number of contigs were from 1239 to 1722. The N50 of the newly sequenced genomes were from 23,732 bp to 44,235 bp. The GC content for the newly sequenced genomes was 48.57%±0.17%.

### Variant Analyses

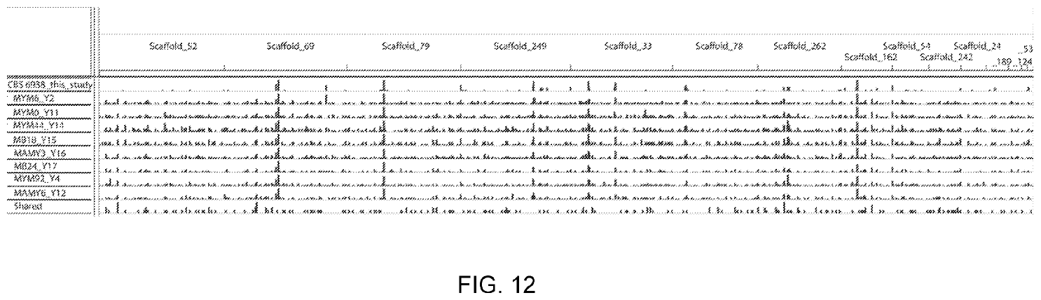

The genome sequence and annotation of X. dendrorhous CBS 6938 was used as a reference to call and annotate the variants (Sharma et al., 2015). After filtering the variants using the criteria detailed hereinabove, there were 983, 1009, 1037, 994, 1015, 977, 1067, and 1091 SNPs and 52, 64, 47, 68, 76, 66, 68, and 61 INDELs detected for the strains MYM6_Y2, MYM0_Y11, MYM44_Y14, MB18_Y15, MAMY3_Y16, MB24_Y17, MYM92_Y4, and MAMY6_Y12, respectively. Next, any mutations detected in the re-sequenced X. dendrorhous CBS6938 compared to the published sequence were removed from the newly sequenced mutant strains. Mutations present across all strains were extracted and used for variant analyses (FIG. 12). After variant annotation, synonymous mutations were removed. These filtering criteria yielded 368 SNPs (see, Table 13). From these mutations, the following criteria were used for further analyses: non-synonymous missense variants in coding regions (144 mutations), upstream variants (126 mutations), upstream variants within 100 bp of a gene (5 mutations), stop gained (2 mutations), start lost (1 mutation), missense/splice variants (4 mutations), and splice donor/intron variants (6 mutations).

### Conserved Variants

Table 13 shows the types of mutations detected across the eight sequence mutant strains, and the genomic details location of the mutation in the chromosome, gene name, protein, gene ID, type of mutation, gene length, protein length, effect, and amino acid change. There were 144 non-synonymous missense mutations in coding regions found across all of the newly sequenced mutant strains. From these mutations, 27 in proteins annotated as hypothetical proteins while the remaining were in more specifically annotated proteins.

Of the mutations in specifically annotated proteins, several were found in subunits of complex proteins. Four SNPs were found in subunits of the gene encoding cytochrome c oxidase: subunit 1, subunit 2, subunit 3. In addition to this cytochrome-related protein, the cytochrome b2 gene encoding protein was also mutated. Five SNPs were found in subunits of the gene encoding to the NADH dehydrogenase protein. Among other mutations in subunits, the ATP synthase subunit 6 gene encoding protein was also mutated. In terms of mutations in genes encoding proteins related to transporters, a small molecule transporter, an ABC transporter, and a sucrose transporter were mutated. Five SNPs in Zn-finger proteins encoding genes were also mutated including C4-type Zn-finger, an uncharacterised MYND Zn-finger, Zn(2)-C6 fungal-type DNA-binding domain gene encoding protein, Zinc finger RING-type, and the related to C2H2 zinc finger protein FLBC. Fatty acid related gene encoding proteins were mutated including the delta 9 fatty acid desaturase, and the acyl-CoA oxidase. From mutations of the TCA cycle, the gene encoding to fumarate reductase was mutated. Mutations were also found in the pantothenate kinase (PanK), the calcium-transporting ATPase, the Snf2 family amino-terminal protein, the 1-aminocyclopropane-1-carboxylate synthase, the cysteine proteinase and the ferredoxin/adrenodoxin reductase encoding genes.

Notably, ‘high impact’ mutations included two SNPs leading to premature stop codons in a WD40 repeat-containing protein and a G protein-coupled receptor, rhodopsin-like, respectively. Additionally, a loss of start codon mutation was detected in one protein annotated as a hypothetical protein. Six mutations within 100 bp upstream of a gene were found including cytochrome b, inositol polyphosphate multikinase, components of the ARGR transcription regulatory complex, mRNA (guanine-n7-)-methyltransferase, and glucosyltransferase-Alg8p; two of these types of effect mutations were annotated as hypothetical proteins. Six mutations with the effect “Splice Donor and Intron Variant” were found. This type of mutation causes the loss of a splicing signal that defines the 3′-end of an exon, the consequence being that the whole intron could be retained as the splicing machinery is unable to recognise the splice donor site (Jian et al., 2013). The mutations found with this effect included the predicted E3 ubiquitin ligase, acid phosphatase, predicted Zn-finger protein, palp-domain-containing protein, Zn(2)-C6 fungal-type DNA-binding domain, and uncharacterised conserved protein. Four mutations were found with the effect missense and splice region variant including mercaptopyruvate sulfurtransferase/thiosulfate sulfurtransferase, ATP-NAD kinase, snf2-family ATP dependent chromatin remodelling factor snf21 with G3713A, and arsenical pump-driving ATPase.

Overall, 25 mutations conserved across the sequenced strains were identified as of particular interest, as set out in Table 14.

### DISCUSSION

### Strain Improvement

In this example, mutagenesis was applied using NTG combined with EMS and UV-light, and incorporated screening methods with selective pressure or cell sorting in the last mutagenic cycle, with the intention of producing mutagenized X. dendrorhous capable of enhanced AX production. This methodology resulted in strains capable of greater than 15-fold AX production as compared to the wild-type strain (e.g., FIG. 7). In addition to improving AX production, the combination of mutagenic agents and screening methods used reduced the number of mutagenic cycles 3-fold as compared to previous studies (see, e.g., Xie et al., 2014).

The use of antimycin, diphenylamine, or β-ionone in our screening methods appeared to assist with the selection of superior strains. Antimycin inhibits the cytochrome P450 enzyme responsible for providing electrons during the oxygenation of the AX molecule by the AX synthase (Bon et al., 1997; Ojima et al., 2006), diphenylamine inhibits the phytoene desaturase enzyme leading to an accumulation of the colourless carotenoid phytoene (Chumpolkulwong et al., 1997), and, p-ionone competes with the p-carotene molecule during its oxygenation by the AX synthase to produce AX (Lewis et al., 1990).

Cell sorting (FACS) was also used as a screening method to select superior strains. FACS used fluorescence properties of the AX molecule to select strains with an improved ability to produce AX. Additionally, as cells screened with FACS were stressed with H₂O₂, cells were selected for increased tolerance to oxidative stresses which is associated with an improved ability to produce AX (Schroeder and Johnson, 1995).

### Culture Media Optimization and Fed-Batch Fermentation

Classical random mutagenesis produces unique strains with specific phenotypic traits making it challenging to directly extrapolate culture media optimization results from other studies. Therefore, one of the selected strains, MYM0, was subjected to a culture media optimization process.

Different nitrogen sources were tested to grow X. dendrorhous cells including urea, ammonium sulphate, yeast extract (YE), malt extract (ME), bactopeptone (BP), and dried corn steep liquor (DCSL) (see Table 2). While urea has been reported to be a low-cost and promising nitrogen source to grow and produce AX in X. dendrorhous (An et al., 2001; Fontana et al., 1996), this example identified that urea inhibited carotenogenesis in our selected strain (Table 2). Ammonium sulphate is another low-cost nitrogen source used to grow X. dendrorhous cells (Flores-Cotera et al., 2001; Ni et al., 2007). The example provided here is consistent with these earlier studies, with ammonium sulphate resulting in the highest growth. YE, ME, and BP are part of the YM media used as a routine to grow X. dendrorhous cells. The evaluation of these media components as a sole nitrogen source confirmed its effectiveness to grow and produce AX. Of note is that ME produced the highest carotenoid content in our selected strain suggesting the importance of this media component. BP delivered improved growth and carotenoid production but its high cost that means this nitrogen source is sometimes replaced by other low-cost sources such as urea or ammonium sulphate (Ni et al., 2007).

Different carbon sources were also tested to grow and produce AX in the selected strain. Table 3 confirms that the selected strains grew in several carbon sources including glucose, sucrose, glycerol, and molasses and can be used to produce high cell densities. The best performing nitrogen and carbon sources were then optimised in a surface response experiment to determine optimal levels to maximise growth and AX production.

X. dendrorhous is a Crabtree positive yeast (high level of sugars produce fermentative products during aerobic fermentations) which limits the application of batch fermentations at a high concentration of carbon source (Reynders et al., 1997). To overcome this limitation, the optimised media and a nonstructure model were used to design the feeding profile for the fed-batch culture. Notably, after seven days of fermentation, the AX production of X. dendrorhous MYM0 increased 50-fold compared with the wild-type strain.

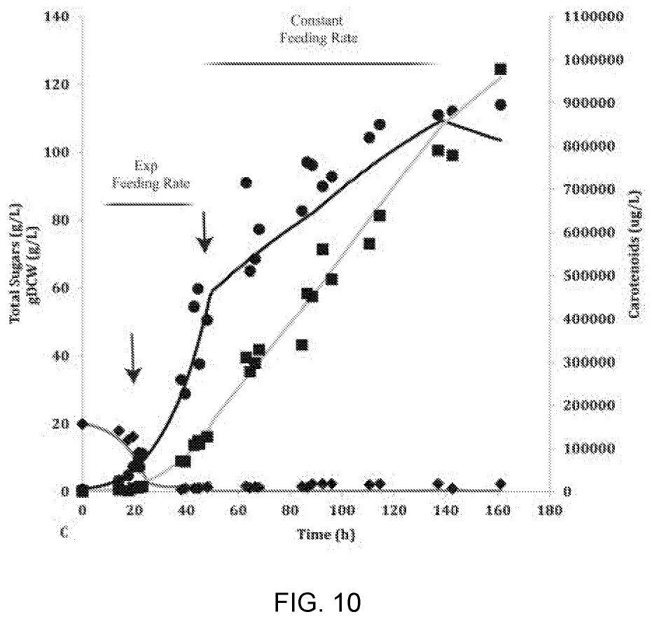

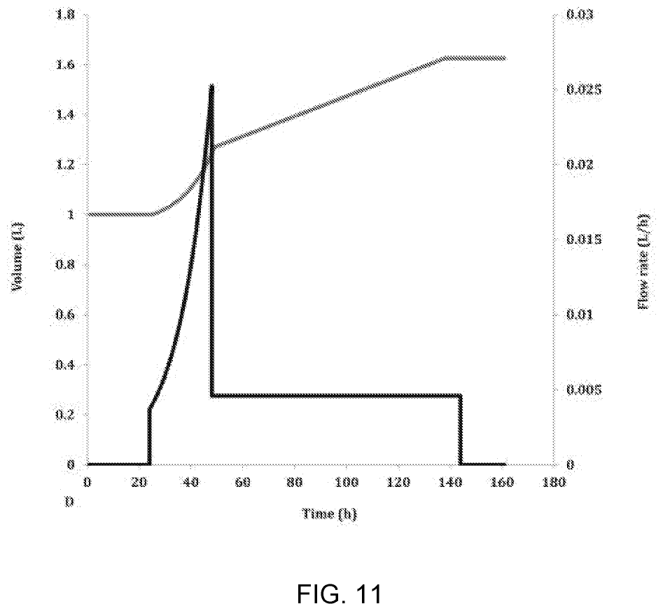

The final biomass achieved was above 100 gDCW/L and the carotenoid content was around 1 g/L (FIG. 10). This result was a 1.41-fold production improvement compared with previous fed-batch cultures that designed the feeding profile using either DO-stat, pH-stat, constant feeding rate, or feedback loops of online sugar measurements (Schewe et al., 2017; Schmidt et al., 2011).

Luna-Flores et al., 2010 used kinetic models to design the feeding profile of a fed-batch culture but the media used in that study was not optimised, rendering 3-fold less growth than in the example presented here. The superiority of our approach highlights the significance of incorporating culture media optimisation in selected strains before using them in fed-batch fermentations.

While glycerol has been reported to be an effective carbon source to stimulate AX production in X. dendrorhous (Silva et al., 2012), and despite glycerol supported growth and production being similar to other carbon sources in a flask fermentation (Table 3), when tested in a fed-batch culture during the maturation phase (after day 3.5 of culture), glycerol in fact accumulated suggesting that it is a poor carbon source for the tested strain. This poor performance when using glycerol in fed-batch fermentations may well be associated with the long adaptation phase prior to assimilation of glycerol (Table 3).

Molasses has previously been tested in X. dendrorhous with two to three times more AX obtained than when using glucose or a synthetic blend of sugars that constitute molasses (Haard, 1988). Our results are consistent with this previous finding with molasses increasing AX production 1.52-fold compared with glucose or sucrose (Table 3) in the tested strain. The stimulation of AX production by molasses suggests it is a promising carbon source to maximise carotenoid production for strains obtained by this approach.

### Genome and Variant Analyses

The genome assembly and filter method (remove contigs below 300 bp and depth below 10X) applied to the selected strains generated genome assembly sizes ranging from 18.55 Mb to 19.11 Mb (Table 4). The genome sizes without filtering the contigs using the mentioned criteria of size and depth resulted in assembly sizes of 19.14±0.15 for all the genomes.

The observed genome sizes were similar to the 19.50 Mb genome of the previously sequenced wild-type X. dendrorhous CBS 6938 strain (Sharma et al., 2015). Similarly, the genome size of the re-sequenced X. dendrorhous CBS 6938 was 19.20 Mb. The GC contents in the new strains and re-sequenced wild-type strain were around 48% which were similar to the 47.3% GC content of the publicly available wild-type strain (Table 4) (Sharma et al., 2015). In previous studies with related strains, X. dendrorhous CBS 7918 yielded a 18.7 Mb genome and 47.2% GC content, and X. dendrorhous CRUB 1149 an 18.9 Mb genome and 47.1% GC content (Libkind et al., 2011).

Genome to genome distance was calculated to objectively calculate the similarity between our selected strains and the wild-type strains (Auch et al., 2010). The genome distance calculated was 0.01 for the re-sequenced X. dendrorhous CBS 6938 and 0.02 for the selected strains (the closer to “0” the more similar the strains in comparison) suggesting that the wild-type strains are more similar between themselves than with our selected strains. This genome comparison confirmed the genome similarity among the X. dendrorhous strains used in this example and those with a genome sequence publicly available.

Genomic Mutations Associated with AX Biosynthesis in X. Dendrorhous

X. dendrorhous uses the precursor isopentenyl pyrophosphate (IPP) for AX biosynthesis, which is generated via the mevalonate (MVA) pathway, starting from acetyl-CoA. Here, a mutation was identified in the pantothenate kinase encoding gene, which is required in the first reaction of the conenzyme A (CoA) biosynthetic pathway (Table 14). Pantothenate kinase phosphorylates panthothenate to form phophopanthothenate at the expense of a molecule of ATP. This reaction is a limiting step in the biosynthesis of CoA, required by the MVA pathway in the form of acetyl-CoA.

The MVA pathway uses five enzymes to produce IPP, in which 3-hydroxy-3-methyl-glutaryl-CoA reductase (HMGR) is a critical regulator and the enzyme that catalyses the production of MVA (Goldstein and Brown, 1990). Here, a mutation was identified in the gene encoding to the HMGR enzyme (see Table 14). HMGR overexpression has been associated with an improvement of AX in X. dendrorhous indicating that the mutation found in the HMGR gene may influence the AX production increase in our selected strains. For example, the addition of ethanol to cultures of the mutant strains X. dendrorhous P-5-6 and Dp-41 overexpressed 3-fold the HMGR gene which was associated with the increase of AX production on those mutated strains (Gu et al., 1997). Similarly, the overexpression of three MVA synthetic pathway genes, namely acetoacetyl-CoA thiolase, HMG-CoA synthase, and HMGR in X. dendrorhous increased AX production 2.1 fold (Hara et al., 2014).

Following the IPP synthesis through the MVA pathway, eight IPP molecules are then condensed through prenyltransferases in which IPP isomerase catalyses the isomerization of IPP to dimethylallyl-pyrophosphate (DMAPP), and then both molecules joined, generating geranyl pyrophosphate (GPP) (Kajiwara et al., 1997). The addition of a second molecule of IPP to GPP gives the precursor C-15 sesquiterpene, farnesyl pyrophosphate (FPP), which is converted into geranylgeranyl-pyrophosphate (GGPP) by a further addition of IPP by the GGPP synthase (see Table 14) (Niklitschek et al., 2008). In this example, the new strains presented a mutation in the GGPP synthase encoding gene, which in abundance has been associated with a remarkable carotenoid production in ripening fruit (Sandmann, 1994). Because GGPPs are the building blocks of the carotenogenesis pathway, the mutation in the GGPP synthase gene might positively influence the synthesis of GGPPs and thus the carotenoid production in our selected strains.

Mutations Associated with Carbon Source and Nutrient Assimilation

This example used sucrose as a carbon source in all screening and selection experiments and efficiently produced high cell densities in a fed-batch culture (FIG. 10). A mutation in a gene encoding a sucrose transporter, associated with sucrose assimilation, was identified. Due to its association with sucrose assimilation, the mutation in the sucrose transporter may have influenced the improved growth and carotenoid production in the selected strain.

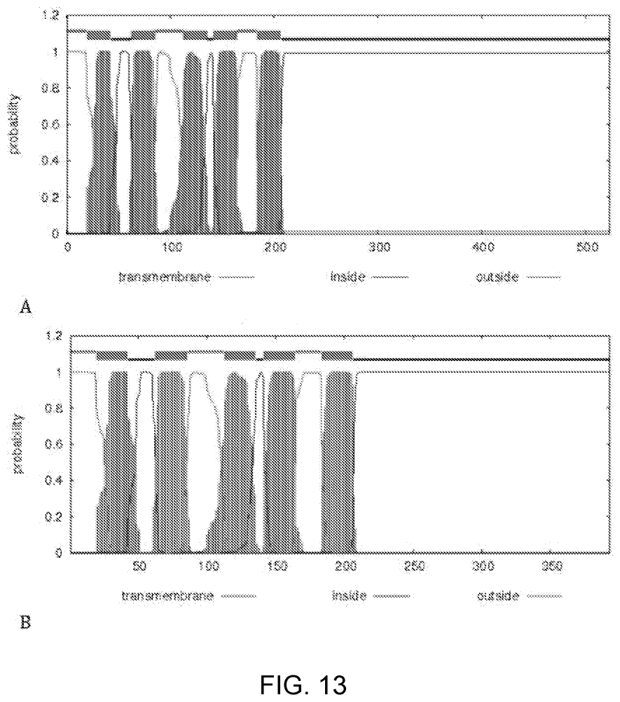

A mutation identified in a gene encoding a G protein-coupled receptor, rhodopsin-like protein (GPCR) had the effect “Stop Gained”. GPCR proteins comprise the largest class of membrane proteins in eukaryote genomes with a common denominator of seven-transmembrane domains. Yeast has three different GPCR proteins for pheromone and sugar sensing (Lengger and Jensen, 2020). Glycerol was a carbon source tested in the selected strains to grow and produce AX (Table 3). For glycerol, the lag phase lasted three days before the culture started to grow. In a fed-batch culture, glycerol failed as it accumulated during its feeding. Potentially, this GPCR mutation is negatively influencing the ability of the selected strain to grow under glycerol as a carbon source. Further analyses in the GPCR mutation using the TMHMM server 2.0 revealed that the transmembrane helices of the GPCR protein were not affected as these were found in the first 200 amino acid sequences—the stop gained was at the position 395/523 (see Table 14 and FIG. 13) (Krogh et al., 2001).

Five mutated genes encoding Zn-finger-type proteins (Table 14), comprising small a protein structural motif characterised by the coordination of one or more zinc ions in order to stabilise the fold, were also identified. These protein types interact with RNA, DNA, or proteins altering their binding specificity for a particular protein. Zn-finger proteins have been associated with the regulation of nitrogen assimilation in Neurospora crassa or Aspergillus nidulans (Fu and Marzluf, 1990), so it is possible that mutations of Zn-finger genes in the selected strain of P. rhodozyma resulted in superior use of ammonium sulphate as compared to urea as a nitrogen source (Table 2).

Mutations Associated with Oxidative Phosphorylation and Stress

Schroeder et. al., 1995 indicated that AX plays an important role against oxidative stress in X. dendrorhous (Schroeder and Johnson, 1995). This oxidative stress link with AX production is also supported by the evidence that AX production in X. dendrorhous is mainly associated with respiratory or aerobic fermentations rather than anaerobic ones (Luna-Flores et al., 2010). ROS tends to be generated during the respiration phase because of electron overflow in the respiratory chain caused by an imbalance of electron transfer during reduction of the ubiquinone pool and electron transfer occurring downstream in the respiratory chain. AX can quench ROS in a manner analogous to superoxide dismutase in which harmful oxygen molecules are break down and eliminated (Schroeder and Johnson, 1993).

ROS are produced via the respiratory electron chain and, in this example, mutations in subunits of the electron transport chain components affecting the complexes I, III, IV, and V (Table 14) were identified. Of these mutations, four were found in subunits of the cytochrome c oxidase (CoC) protein. This CoC protein is responsible for carrying the electrons from complex III to complex IV in the electron transport chain, the last step in the electron transport chain. In fact, this complex is inhibited by antimycin or potassium cyanide (KCN), a compound used in our studies as a screening method.

Evidence suggesting that X. dendrorhous shifted from KCN-sensitive to KCN-insensitive growth in later growth phases (carotenoid production phases) (Schroeder and Johnson, 1993) supports the importance of alternative pathways of oxygen utilization during AX biosynthesis. Another report suggests that mutant strains sensitive to antimycin, can create an imbalance in the flow of electrons during the electron transport chain, increasing the amount of ROS and so improving AX production (An et al., 1989). This overflow of electrons can also increase AX production by the action of AX synthase, an enzyme that belongs to the P450 family enzyme and requires an electron donor (P450 reductase or cytochrome b), to incorporate the oxygen functional groups to the molecule of p-carotene.

In this example, a mutation of a ferredoxin/adrenodoxin reductase encoding gene (Table 14) was also observed. Adrenodoxin is a cAMP-regulated ferredoxin that transports electrons from NADPH-dependent adrenodoxin reductase to P450 family enzymes (Grinberg et al., 2000). The electron donor capability of ferredoxin/adrenodoxin reductase that suggests a potential role as an electron donor required by AX synthase. Other mutations associated with the electron transport chain included a mutation in the cytochrome b, cytochrome b2 and ATP synthase encoding genes and five mutations in subunits of the NADH dehydrogenase protein (Table 14). These mutations are potentially associated with the improved AX production in the selected strains due to the abundance of oxidative phosphorylation enzymes such as NADH dehydrogenase and ATP synthase, found when X. dendrorhous cells were growing on succinate as a carbon source, and where production of AX was around 2-fold more than when grown on glucose (Martinez-Moya et al., 2015).

Another mutation potentially associated with increased AX production in the selected strains is in an acyl-CoA oxidase encoding gene (Table 14). This enzyme is associated with the fatty acid metabolism in a reaction involving the oxidation of acyl-CoA to tans-2,3-dehydroacyl-CoA and the reactive oxygen species H₂O₂, which can trigger an increase of AX production to protect the cells from an oxidative stress (Liu and Wu, 2006).

Another mutation associated with lipid metabolism was observed in a delta 9 fatty acid desaturase, which is reported to have preferences for substrates of C18:1 and C16:1 converting to C18:2 and C16:2 fatty acids (L. Zhang et al., 2020). Notably, the most abundant fatty acids in the X. dendrorhous pathway are linoleate (C18:2), stearic acid (18:0), oleic acid (C18:1), palmitic acid (C16:1), and hexadecanoic acid (16:2) (Sharma et al., 2015). The proportion of fatty acid and its influence to produce AX (Miao et al., 2011) suggests that the mutation in the delta 9 fatty acid desaturase encoding gene may stimulated AX production in our selected strains of X. dendrorhous.

It is further notable that, in this example, antimycin, β-ionone, diphenylamine, or the stressor H₂O₂ were used in the screening methods to select superior strains of X. dendrorhous, which potentially increased selection for strains with the mutations in the electron transport chain to enhance AX production.

### Example 2: Metabolomic and Transcriptomic Analysis of a High Astaxanthin Producer Strain of Xanthophyllomyces dendrorhous

In this example, one of the X. dendrorhous mutant strains developed as per Example 1, and the wild-type as a control, were cultured in chemically defined media and instrumented fermenters, and differential kinetic, metabolomics, and transcriptomics data were obtained. The results obtained in this example suggest that carotenoid production primarily occurred during growth phase.

During the growth phase, the mutant strain showed positive regulation of central carbon metabolism metabolites associated with glycolysis, the pentose phosphate pathway, the TCA cycle, and amino acid and fatty acid biosynthesis. In the stationary phase, amino acids associated with the TCA cycle increased, but most of the fatty acids and central carbon metabolism metabolites decreased. TCA cycle metabolites such as succinate, fumarate, and a-ketoglutarate were abundant during both growth and stationary phases.

The overall observed metabolic changes in the central carbon metabolism and abundance of TCA cycle metabolites suggest an enhancement in the electron respiratory chain in the mutant, and in the provision of the electrons required for the AX synthesis by the AX synthase, may be primarily responsible for enhanced AX production. Transcriptomic data correlated with the metabolic data and found a positive regulation of genes associated with the electron respiratory chain.

### Materials and Methods

### Strains, Media, and Growth Conditions

The wild-type strain of X. dendrorhous CBS 6938 and the mutant strain X. dendrorhous MYM0, each as described in Example 1, were used in this example. In this example X. dendrorhous MYM0 is hereinafter referred to as X. dendrorhous BPAX-A1. Storage of the strains was as described in Example 1. Chemically defined media for inoculum and fermenters contained (in g/L): glucose (20), (NH₄)2SO₄ (6), KH₂PO₄ (2), FeSO₄·7H₂O (0.019), MgSO₄·7H₂O (0.88), and CaCl-2H₂O (0.2). Potassium hydrogen phthalate 20 was used as a buffer when growing in flasks. The pH was adjusted to 5.5 with NaOH 2M. The media was supplemented with trace salts (mg/L): ZnSO₄·7H₂O (5.01), CuSO₄·5H₂O (0.75), MnSO₄ (0.48), H₃BO₃ (0.6), Na₂MoO₄·2H₂O (0.6), and KI (0.15) and vitamins (mg/L): myo-inositol (60), vitamin B3 (niacin) (3), vitamin B5 (pantothenic Acid) (3), vitamin B1 (thiamine) (3), vitamin B6 (pyridoxine) (3), vitamin B7 (biotin) (0.048), and p-aminobenzoic acid (1.8).

### Analytical Techniques

### Optical Density (A600)

OD was measured as for Example 1.

### Dried Cell Weight

Dried cell weight was measured as for Example 1.

### Total Carotenoids

Total carotenoids were assessed as for Example 1.

### Hplc Analyses for Astaxanthin and Other Carotenoids

HPLC analyses for astaxanthin and other carotenoids were performed as for Example 1.

### Hplc Analyses for Sugars and Organic Acids

Supernatants for sugar and organic acid analyses was obtained by centrifugation of 1 mL of fermentation sample at 15,000 rpm for 5 min. Organic acids and carbohydrates were quantified by ion exchange chromatography using an Agilent 1200 HPLC system and an Agilent Hiplex H column (300×7.7 mm, PL1170-6830) with a guard column (PL Hi-Plex H 50×7.7 mm, PL1170-1830). Sugars were monitored using a refractive index detector (Agilent RID, G1362A) set on positive polarity and optical unit temperature of 35° C. while organic acids were monitored at 210 nm (Agilent MWD, G1365B). 30 gL of each sample was injected onto the column using an auto-sampler (Agilent HiP-ALS, G1367B) and the column temperature was kept at 40° C. using a thermostatted Column compartment (Agilent TCC, G1316A). Analytes were eluted isocratically with 5 mM H2SO4 at 0.4 mL/min for 40 min. Chromatograms were integrated using Chromeleon 7.2 software.

### Intracellular Metabolite Extraction and Analyses

The following methods for quenching, extraction, and analyses were adapted from (Canelas et al., 2008, 2009; Martinez-Moya et al., 2015; Luna-Flores et al., 2018; Pan et al., 2020). Cells were quenched in cold methanol 60% and placed in a bath of ethanol/dry-ice. Cells were then centrifuged at 4,500 rpm for 5 min at −20° C. and pellet was washed with cold 60% methanol before to snap-freeze in liquid nitrogen. Cell pellets were kept at −80° C. until further use. The pellet was then dissolved in a mixture 1:1 of chloroform and methanol 50% and submitted to five cycles of 5 min of bead beating using acid washed glass beads (Sig. Cat. No. G1152-100G) and a tissue lyser (Qiagen TissueLyser II); the tubes were cooled down on ice for 5 min before start each cycle.

To separate polar and non-polar metabolites, the disrupted cells were centrifuged at 15,000 rpm for 15 min in which polar metabolites were in the top layer (methanol 50%) and non-polar metabolites were in the bottom layer (chloroform). The top layer was collected, freeze dried, and the pellet obtained was finally re-suspended in 2% acetonitrile. 5 uM of AZT, 3 ppm of 13-C valine, and 3 ppm of 13-C sorbitol were used as internal standards. LCMS was used to analyse central carbon metabolism using the method described in (Luna-Flores et al., 2018). Briefly, analyses were performed using a Dionex Ultimate 3000 HPLC system coupled to an ABSciex 4000 QTRAP mass spectrometer. Liquid chromatography was performed using a 50 min gradient with 0.3 mL/min flowrate, on a Phenomenex Gemini-NX C18 column (150×2 mm, 3 gm, 110 A), with a guard column (SecurityGuard Gemini-NX C18, 4×2 mm), and column temperature of 55° C. The mobile phases used were: 7.5 mM aqueous tributylamine (Sigma-Aldrich) with pH adjusted to 4.95 (+0.05) using acetic acid (Labscan) for Solvent A, and acetonitrile (Merck) for Solvent B. Samples were kept at 4° C. in the autosampler and 10 gL of various dilutions of samples were injected for analyses. The HPLC was controlled by Chromeleon 6.80 software (Dionex). Mass spectrometry was achieved using a scheduled multiple reaction monitoring (sMRM) method on the negative ionisation mode. Collected data were processed using MultiQuant 2.1 (AB Sciex). The amino acids profile was obtained using the protocol described in (Chen et al., 2020). In brief, Shimadzu LCMS 8050 was used for amino acid analyses. This instrument was equipped with three quadrupoles for mass analysers and collision. Liquid chromatography was performed by injecting 1 uL of sample to a F5 column (Sigma) and eluted in a 25 min gradient using as mobile phase acetonitrile with 0.1% of formic acid at 0.25 mL/min. Oven was set to 40° C. Electrospray ionization was used to ionise the sample. Mass spectrometry was achieved using a scheduled multiple reaction monitoring (sMRM) method on the positive ionisation mode. Amino acid mix was used as standard for identification and quantification (Sig. Cat. No. A9906-1ML). Skyline daily was used to analyse amino acids detected in LC-MS (Adams et al., 2020). The fatty acid profile of yeast biomass was determined using the following procedure. A portion of dried yeast biomass (50 mg) was mixed with 2 mL solvents containing methanol/hydrochloric acid/chloroform (10:1:1, v/v/v). The mixture was heated at 90° C. for 1 h in a sealed glass tube to convert microbial oils to fatty acid methyl esters (FAMEs). Then the mixture was treated through mixing with 0.9% NaCl solution (1 mL). Afterwards, FAMEs were extracted through addition of 0.5 mL hexane, followed by centrifugation. Then, the supernatant hexane phase containing FAMEs were analyzed by an Agilent 6890 Series Gas Chromatography system equipped with a HP 5973 mass spectrometer detector and a HP-5MS capillary column (Agilent J&W 30 m×0.25 mm×0.25 [im). 1 [J.L of the sample was injected with a split ratio of 10:1. The injection port temperature was 230° C. Initial column temperature was 90° C. and held for 1 min, followed by increasing the column temperature at a rate of 15° C./min until 180° C., 5° C./min to 220° C., 10° C./min until 250° C. and held for 10 min. FAME Mix (Supelco® 37 Component, Sigma-Aldrich) was used as standard.

### Rna Extraction, Sequencing, and Analyses

Cells sampled at Phase III were used for RNA extraction and analysis. For this, cells (50 ODs) were quenched in cold methanol 60% and placed in a bath of ethanol/dry-ice. The cells were then centrifuged at 4,500 rpm for 5 min at −20° C. and the pellet was snap-frozen in liquid nitrogen and finally storage at −80° C. until further use. ZymoBiomics DNA/RNA Miniprep Kit (Zymo Research Cat. No. R2002) was used for RNA extraction. For this, the pellets were first submitted to a cryogenic grinding step using a mortar, pestle and liquid nitrogen. To ensure extraction, using indications of the kit, the grinded pellet was submitted to three cycles of five minutes of bead beating using a Qiagen Tissue Lyser II. This grinded extract was then used for RNA extraction following the indications of the kit. Extracted RNA was quantified using a NanoDrop and Qubit 4.0. The library was prepared using the Illumina Stranded mRNA prep kit. The quality of the RNA and library were evaluated by a Fragment Analyser 5200. Finally, the samples were sequenced using the Illumina platform 100 bp Pair End using NovaSeq 6000 and a SP PE100 flow cell. Quality of the reads was evaluated using FASTQC (Andrews, 2010) and Trimmomatic (Bolger et al., 2014) was used to remove bad quality reads. Then, Tophat, Cufflinks, and CuffDiff were used to align the RNA-seq reads against the reference genome X. dendrorhous CBS6938 (Sharma et al., 2015), normalize and annotate the transcripts, and evaluate the differential expression, respectively (Trapnell et al., 2012). The cutoff for significant differentially expressed genes was q<0.05.

### Statistical Analyses of Intracellular Metabolomics

Metabolomics data were normalized and analysed for statistical significance (De Livera et al., 2012). Principal component analyses were used to profile all metabolites (Mendez et al., 2019).

### Instrumented Fermenters

Instrumented fermenters were performed in a 1 L New Brunswick BioFlo/CelliGen 115. The fermenter was configured with one six-blade Rushton impeller with a diameter of 2.5 cm, three baffles, one ring sparger, and ports for acid, base, antifoam, and sampling. The fermenters were equipped with probes and controllers of pH, pO₂, temperature, and antifoam to measure and control these parameters, respectively. Optimal fermenter conditions were the following. The fermenter was inoculated with 10% of cells at A600 of 5 growing at the mid-exponential phase. The pH was controlled at 5.5 using 2 M sodium hydroxide or 2 M of sulphuric acid. The foam was controlled by adding Antifoam C (Sigma Cat. No. A8011). The temperature was controlled at 20° C. using a heater jacket or chiller. The dissolved oxygen was controlled at 70% of air saturation by using cascade changes in agitation (400-1200 rpm), and a constant air flow rate of 1 VVM.

### Calculation of Fermentation Parameters

Specific growth rates (p) was calculated across all the growth phases using the logarithm method. Yield of sugar conversion to biomass (Yxs) was calculated using the total biomass produced over the consumed substrate. The specific consumption rate of glucose (qs) and the specific production rates of carotenoids (qp) and AX (qpax) were computed at all the growth phases by multiplying the specific growth rate by the linear correlations of sugar or carotenoids and AX with biomass. For the stationary phase in which no-growth was observed, the specific rates were calculated using the linear correlations of sugar or carotenoid production with an average of biomass multiplied by its time frame.

### Results

### Instrumented Fermenters and Kinetic Analyses

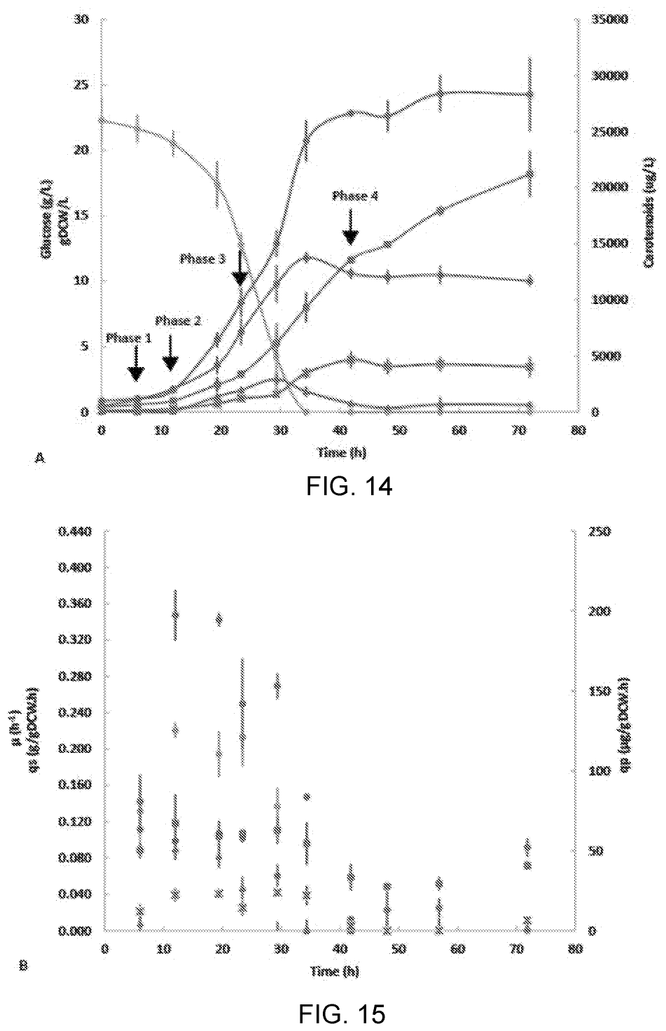

The wild-type X. dendrorhous CBS 6938 and the mutant X. dendrorhous BPAX-A1 (referred to as MYM0 in Example 1) strains were grown for three days in a chemically defined media and sampled for growth and production measurements. Comparatively, as can be seen in Table 9, the wild-type final biomass was 13.60±0.29 gDCW/L, which represented 35.8% higher than that obtained in the mutant strain. The wild-type sugar to biomass yield (Yxs) was 0.62 g/gDCW being 44% higher than that obtained in the mutant strain. In terms of AX production, the mutant strain presented an improvement of around 11-fold compared to the wild-type strain. The final proportion of AX was around 70% for both strains. In terms of specific rates, as can be seen in Table 10 and FIGS. 14-17, the wild-type strain had a maximum specific growth of 0.12 h⁻¹ which was 9% higher than that obtained in the mutant strain. The mutant and wild-type strain did not present a lag growth phase, but in the total carotenoid production (see FIGS. 14-17). The maximum specific carotenoid production in the mutant strain was 12-fold higher than that obtained in the wild-type strain. In terms of strain specific changes for growth and production, as can be seen in Table 10 and FIG. 14, the mutant strain had an increase of the specific carotenoid production of 2.44-fold from Phase 1 to Phase 2 and continued until the stationary phase. Interestingly, the specific AX production remained relatively constant across all the growth phases. At the stationary phase, the carotenoid production ceased but the accumulation of AX continued mainly due to an increase in the AX composition of the total carotenoids. For the wild-type strain, the specific carotenoid production was increased 7.92-fold from Phase 1 to Phase 2 and it was extended until the stationary phase. For the AX production specifically, it was increased 2.37-fold and 7.51-fold from Phase 1 to Phase 2 and Phase 3, respectively.

### Metabolite Profiling of Wild-Type and Mutant Strains of X. Dendrorhous

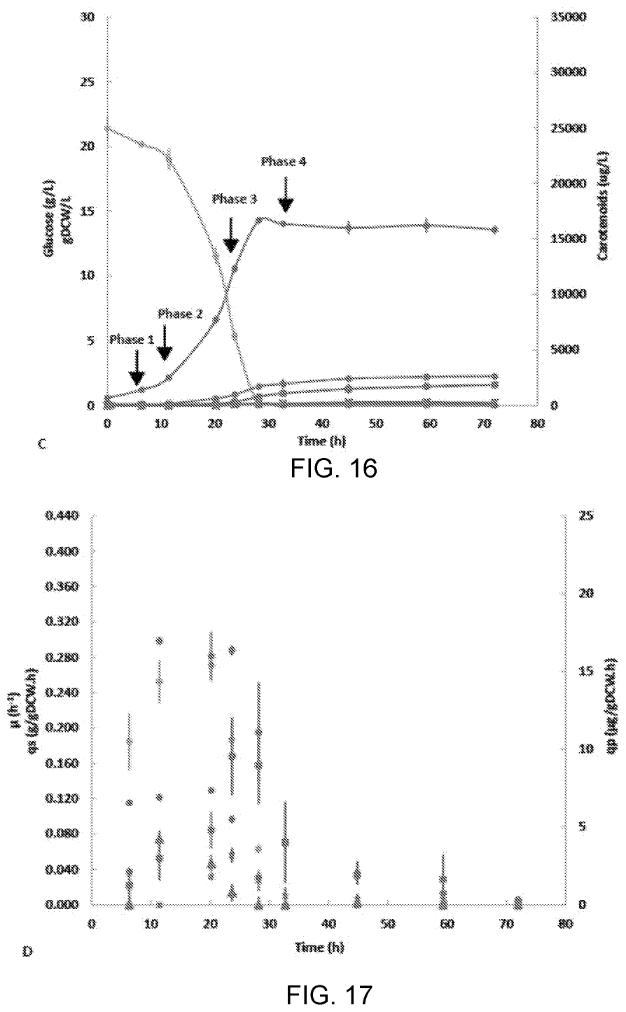

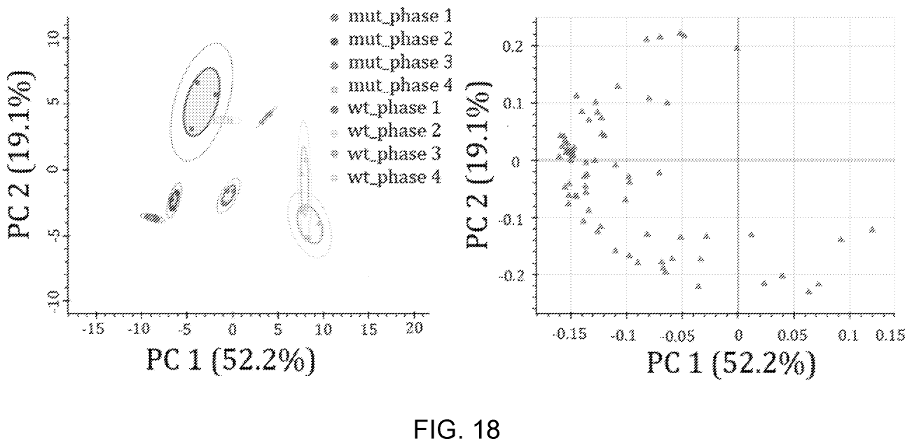

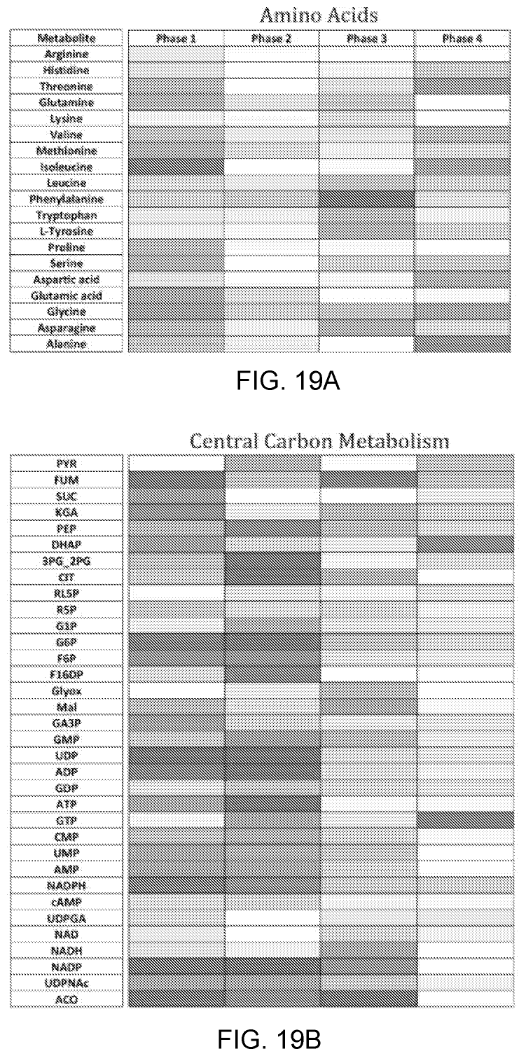

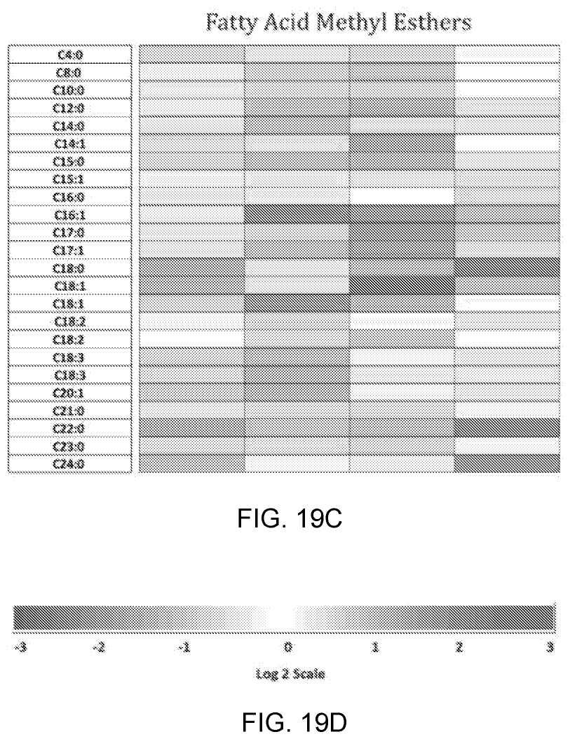

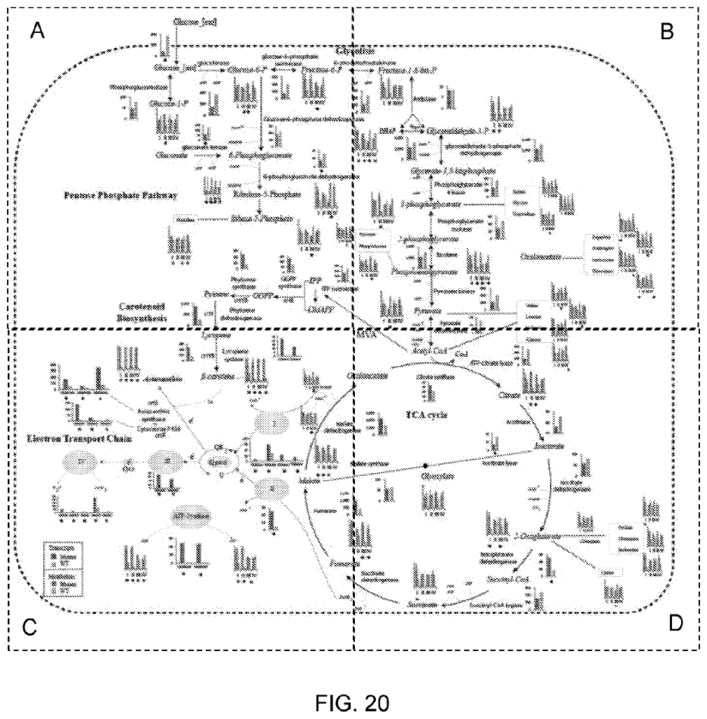

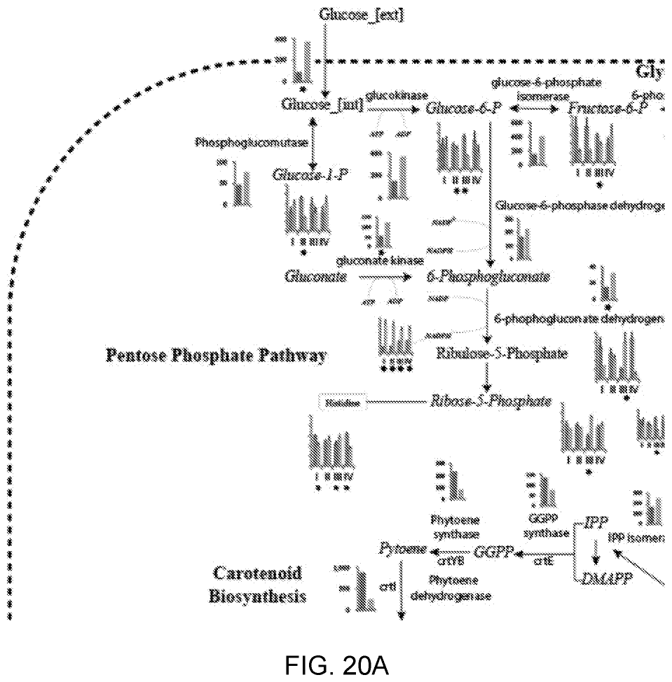

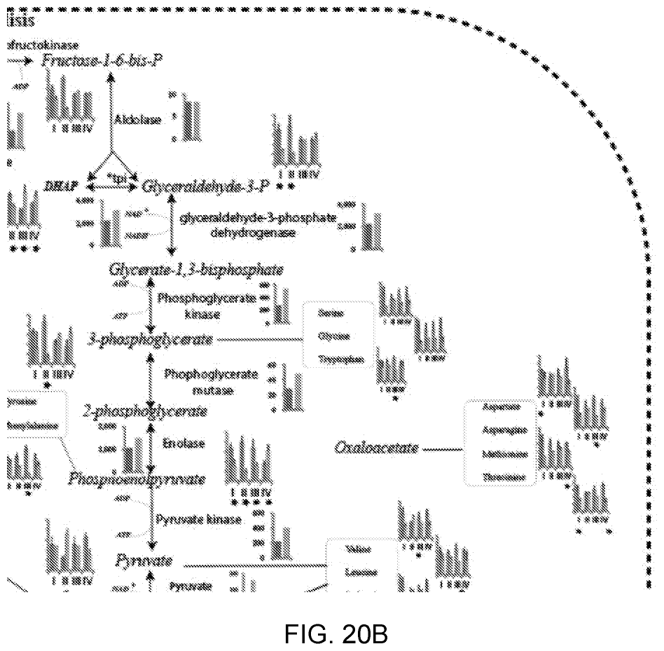

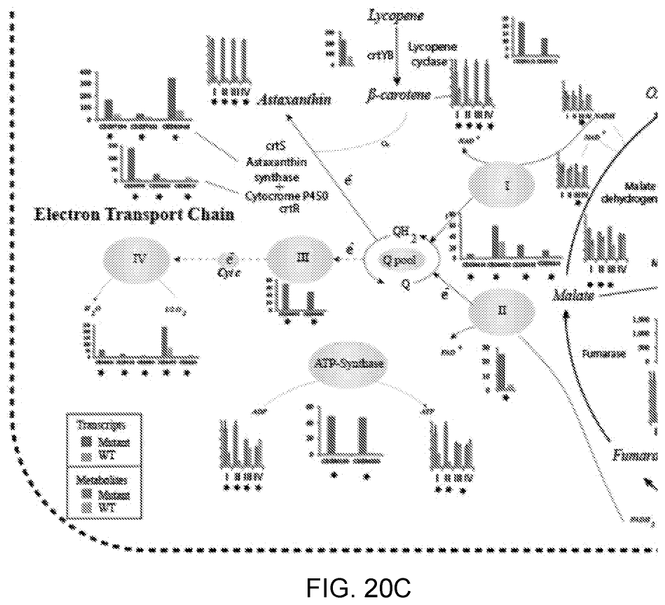

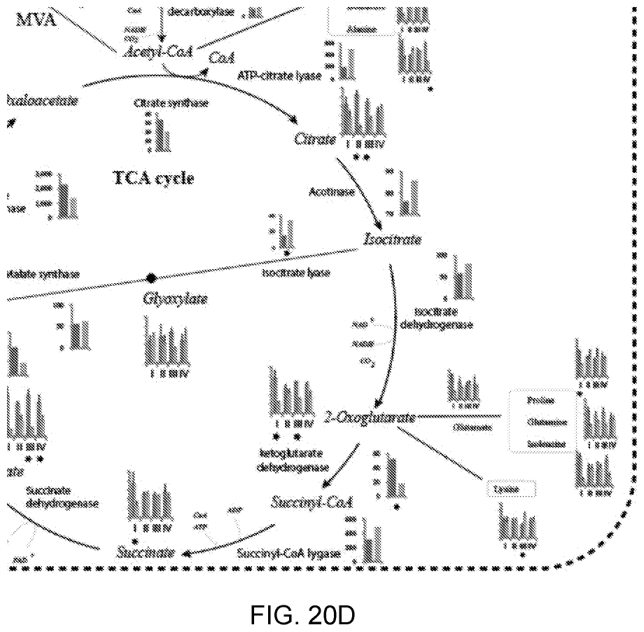

FIGS. 14 and 16 show the growth phases sampled for intracellular metabolite analyses. A total of 80 metabolites associated with primary metabolism were obtained. Before statistical analyses, the metabolite data was normalized based on biomass and internal standards. Principal component analyses (PCA) was then used for statistical grouping of the samples. As can be seen in FIG. 18, the score and loading plots discerned between the different metabolites analysed grouping the four growth phases of the strains under analyses. Phase 3 suggested to be the most explanatory for differential comparison due to it was isolated inside each strain and between them. To compare the relative amounts of metabolites, the response ratio of each metabolite was obtained and converted to log 2 (FIG. 19). The metabolomes of the wild-type and mutant strains were then compared at each growth phase. Here, central carbon metabolism metabolites presented the highest differences (more abundant in the mutant strain) for the first three growth phases analysed. In Phase 4, except pyruvate, fumarate, succinate, a-ketoglutarate, ribulose-5-phosphate, and glucose-6-phosphate all metabolites were similar or less abundant in the mutant strain. A similar pattern was observed with FAME metabolites, in which methyl laurete (C15:0) and methyl palmitoleate (16:1) were more abundant in all the growth phases. In regards to amino acids, they were more abundant in the mutant strain during all the growth phases. To study the dynamic of metabolite profiles, the principal carbon, amino acid, FAME, electron transport chain, and carotenogenesis pathways were obtained using the KEGG database; to facilitate the comparison, the response ratios were normalized to 100 (FIG. 20).

### Comparative Transcriptomics Analysis

Cells from Phase 3 were used for differential transcriptomic analysis. RNA-sequencing was used to compare the transcriptional profile of the strains. Our analyses identified 6363 transcribed genes, of which 690 were significantly different (q<0.05). From these genes, 211 (132<Log 2 −2) were downregulated and 479 (228>Log 2 2) were upregulated (Table 16). FIG. 20 shows the gene expression on the pathways glycolysis, pentose phosphate pathway (PPP), TCA cycle, electron transport chain, and carotenoid biosynthesis (see also Tables 15 and 16; FIG. 20). Among the most relevant changes, the hexose carrier gene CED82529 was significantly downregulated in the mutant strain (Log 2 −2.10, q<0.05). There were not found significant differences on the expression of genes associated with the glycolytic pathway. However, this pathway seems to be more active in the wild-type strain than in the mutant strain. Similarly, the gluconate kinase and 6-phosphogluconate dehydrogenase PPP genes were significantly downregulated in the mutant strain (FIG. 20 and Table 17). For the TCA cycle, the gene isocitrate lyase was significantly downregulated (Log 2 −1.24, q<0.05) in the mutant strain and the genes ketoglutarate dehydrogenase and succinate dehydrogenase were significantly upregulated (Log 2 1.55, q<0.05) and (Log 2 2.8, q<0.05), respectively. Several genes associated with the electron transport chain were significantly upregulated in the mutant strain including the Complex I genes (CDZ96154, CED84925, CDZ96153, and CDZ96151), Complex II gene (CDZ98193), Complex III genes (CED80059 and CED80058), Complex IV genes (CED800856, CDZ96152, CED800061, CED84572, and

CED82283), and ATP-Synthase genes (CDZ96150 and CDZ96333) (see Table 17 and FIG. 20 for more details). Similarly, most of the genes associated with the AX biosynthesis were upregulated in the mutant strains including the AX synthase CDZ97194 (Log 2 2.23, q<0.05), the aldo/ketolase related proteins CDZ97194 (Log 2 1.85, q<0.05) and CDZ97021 (Log 2 1.13, q<0.05), and the P450 reductase CED84998 (Log 2 4.05, q<0.05) and CDZ98632 (Log 2 1.44, q<0.05); from these P450 reductase genes, the CED85015 belonging to the CYP2 family was significantly downregulated (Log 2 −2.30, q<0.05).

### DISCUSSION

In Example 1, comparative genomics was explored to link phenotype and genotype of the mutant strain X. dendrorhous BPAX-A1 (MYM0). However, metabolic changes associated with improved AX production remained unclear. In this example, to gain metabolic insight, the wild-type and mutant strains were grown in chemically defined media and sampled at four growth phases for kinetic and metabolic comparisons. No substantial lag-phase was observed in either strain, and carotenoid production appeared primarily associated with growth. Principal Component Analysis (PCA) of all metabolites grouped the growth phases between and among the strains under comparison. Growth Phase III was fully isolated in the PCA analysis (see FIG. 18), and kinetically, presented the highest carotenoid production rate (see Table 10), indicating that this phase may be particularly relevant for transcriptomic comparisons. Accordingly, RNA-seq data was obtained and integrated with kinetic, metabolomics, and genomics data.

### Comparative Metabolomics

To study metabolic changes, a system-level characterization was performed, previously shown to be useful (Luna-Flores et al., 2016, 2018). Extracellular and intracellular metabolites were measured across the fermentation time course (FIGS. 14-17). As can be seen in Tables 9 and 10, the mutant strain produced more carotenoids than the wild-type strain but had a lower specific growth rate and final biomass. These results are in agreement with other studies in which strains with enhanced AX production have displayed lower biomass (Cannizzaro et al., 2004; Gassel et al., 2013). These previous studies suggested that wild-type X. dendrorhous used the PPP primarily for glucose degradation, leading to greater abundance of NADPH and pentoses used for lipid and nucleotide biosynthesis, respectively.

In this example, metabolites associated with the PPP were more abundant in the wild-type strain than in the mutant strain (FIG. 19 and FIG. 20). Of note is that NADPH was lower in the mutant than in the wild-type strain, providing a possible (at least partial) explanation for the observed decreased biomass. This decreased NADPH may well be associated with the improved AX in the mutant strain, with production of isoprenoid units of the AX molecule through the MVA pathway potentially competing with NADPH production. Also, the wild-type strain had a maximum glucose uptake rate 13% higher than the mutant strain. Notably, the hexose carrier gene CED82529 was significantly downregulated (Log 2 −2.10, q<0.05) in the mutant strain (Table 16 and Table 17), which may well be at least partly responsible for the lower growth rate in the mutant strain as compared to the wild-type strain.

During the growth phase, the mutant strain showed abundance of metabolites associated with glycolysis, TCA cycle, amino acids, and fatty acids metabolites, but at the stationary phase, except for amino acids, most of these metabolites were more abundant in the wild-type strain. Similarly, metabolites of the TCA cycle namely succinate, fumarate and a-ketoglutarate were more abundant in the mutant strain than the wild-type strain during all the growth phases studied (FIG. 19). These metabolic changes suggest a more active central carbon metabolism to grow and produce AX in the mutant strain in which the TCA cycle is playing an important role. The supplementation of TCA cycle metabolites, such as succinate or citrate, has been reported to increase AX production in X. dendrorhous (Flores-Cotera et al., 2001; Martinez-Moya et al., 2015). Similarly to oleaginous yeast, X. dendrorhous can use citrate as a carbon source for lipids and carotenoids biosynthesis (Flores-Cotera et al., 2001). It is reported that the use of succinate as a sole carbon source increased TCA cycle metabolites and proteins when compared to glucose as a carbon source suggesting that succinate can positively alter the whole synthesis of metabolites and translation of proteins (Martinez-Moya et al., 2015).

In regard to lipids, linoleic acid, palmitoleic acid, and stearic acid were the most abundant fatty acids in both strains. At Phase IV or stationary phase, the mutant strain presented 1.15-fold more palmitoleic acid, 1.17-fold more steric acid, and 1.41-fold less linoleic acid than the wild-type strain. Similarly to linoleic acid, all the other fatty acids were decreased in the mutant strain suggesting changes in the final fatty acid profile between the strains under study. Also, amino acids associated with the TCA cycle were more abundant in the mutant strain than in the wild-type strain. This Phase IV also matched with a switch in pH control including use of acid instead of base to maintain the pH at the desired level. The production of carotenoid ceased but AX continued increasing mainly due to a change in the composition of the total carotenoids. All collected data at Phase 4 suggest that the mutant strain was consuming lipids and peptides to obtain energy and metabolic precursors to continue producing AX.

### Integrating Transcriptomics Respiratory and Energy Metabolism

For Example 1, a genomic variant analysis was performed in mutant strains including X. dendrorhous BPAX-A1. This genomic analysis found mutations in the electron respiratory chain pathway and metabolomics analysis suggested changes in the TCA cycle associated with the electron respiratory chain. In Example 1, a subset of mutations that were considered of particular interest were identified. Notably, some of these mutated genes were overexpressed based on transcriptomic analysis (see Table 15, Table 16, and Table 17). For example, a mutation in the succinate dehydrogenase or complex II gene (CDZ981393) was identified; this gene was significantly upregulated in the mutant strain (Log 2 2.8, q<0.05). Also, the complex I or NADH:ubiquinone/plastoquinone oxidoreductase, chain 3 (CDZ96154) gene was mutated with two intron variants and this gene was significantly upregulated in transcriptomics (Log 2 4.73, q<0.05). The complex I or the NADH dehydrogenase subunit 4 (CDZ96151) gene had two mutations and was significantly upregulated in transcriptomics (Log 2 4.13, q<0.05). Similarly, cytochrome b gene CED80058 was mutated in the upstream and in the downstream of the gene; these mutations might be responsible for the significantly upregulated gene CED80058 in transcriptomics (Log 2 5.15, q<0.05) (FIG. 4). The complex IV or cytochrome c oxidase subunit 1 gene CED80056 had two mutations, the cytochrome c oxidase subunit 2 gene CDZ96152 had one mutation, and the cytochrome c oxidase subunit 3 gene CED80061 had two mutations. These genes were significantly upregulated in transcriptomics: CED80056 (Log 2 5.57, q<0.05), CDZ96152 (Log 2 5.30, q<0.05), and CED80061 (Log 2 5.01, q<0.05). Linked to these changes in the Complex IV gene, the cooper transporter gene CED82663 was significantly upregulated (Log 2 2.38, q<0.05) in the mutant strain probably increasing the copper availability inside the cell. As Complex

IV protein contains two copper catalytic centres (Cua and Cub), copper supplementation has been associated with an increase of AX production in X. dendrorhous (Srinivasan and Avadhani, 2012; Martinez-Cardenas et al., 2018). The ATP synthase subunit 6 gene CDZ96150 had two mutations in the upstream of the gene, and two other mutations in the downstream of the gene. Similarly, the ATP synthase subunit mitochondrial gene CDZ96333 had two mutations in the downstream of the gene. These CDZ96150 and CDZ96333 genes were significantly upregulated in transcriptomics (Log 2 5.25, q<0.05) and (Log 2 4.65, q<0.05), respectively. In X. dendrorhous, AX is utilized by oxidation of P-carotene through the P450 enzyme AX synthase (Ojima et al., 2006). This reaction needs the adjunct activity of the cytochrome P450 reductase or the cytochrome b as an electron donor (Alcaino et al., 2008a). Although no mutation was found in the AX synthase gene (CED83940), this gene was significantly upregulated in transcriptomics (Log 2 2.23, q<0.05).

Similarly, the cytochrome P450 CYP3/CYP5/CYP6/CYP9 subfamilies gene (CED84998) and the cytochrome P450 CYP4/CYP19/CYP26 subfamilies gene (CDZ98632) were significantly upregulated (Log 2 4.05, q<0.05) and (Log 2 1.45, q<0.05), respectively. Interestingly, the cytochrome P450 CYP2 subfamily (CED85015) was significantly downregulated (Log 2 −2.30, q<0.05). Cytochrome P450 reductase (crtR) of X. dendrorhous has been cloned and found to be an essential gene for AX biosynthesis (Alcaino et al., 2008b). That study suggested that only one crtR is required for AX biosynthesis. However, the disruption of that gene in X. dendrorhous was not lethal suggesting the existence of an alternative electron donor such as the cythochrome b5. Here, upregulation of two crtR genes (CED84998 and CDZ98632) and downregulation of another one (CED85015) was observed. No significant overexpression of cytochrome b5 was observed.

Significant upregulation of the Ferredoxin/adrenodoxin reductase gene (CDZ98521) (Log 2 −2.30, q<0.05) was observed. Ferredoxin/adrenodoxin reductase can transport electrons from a FADH2 coenzyme, produced during p-oxidation of lipids by the action of the acyl-CoA dehydrogenase, to a P450 systems of the mitochondria (Hanukoglu, 1992). Linked with this reaction step, here significant upregulation of an acyl-CoA dehydrogenase gene (CED84717) (Log 2 1.36, q<0.05) was observed, which is involved in the initial step of p-oxidation of lipids and production FADH2. This upregulated lipid degradation to provide FADH2 cofactors is potentially associated with the ongoing change in proportion of AX in the mutant strain BPAX-A1 after glucose was depleted and in its change of final lipids profile in which linoleic acid was 1.41-fold less than the wild-type.

Although not significant, all the other carotenoid-associated production genes (GGPP synthase, phytoene synthase, phytoene dehydrogenase, and lycopene cyclase) were nominally upregulated in transcriptomics in the mutant strain BPAX-A1 (Table 17). Linked to the TCA cycle, the a-ketoglutarate dehydrogenase gene (CED83799) was upregulated in transcriptomics in the mutant strain (Log 2 1.55, q<0.05) and downregulated the isocitrate lyase gene (CED85129)(Log 2 1.24, q<0.05). This suggests that the mutant strain is not using the glyoxylate shunt to deal with cell oxidative stress (Ahn et al., 2016), but the improved amounts of AX produced. Also, the asparagine synthase gene (CED83843) was upregulated in the mutant strain (Log 2 2.11, q<0.05). Asparagine is a TCA cycle derived (from oxaloacetate) amino acid which was more abundant in the mutant strain than in the wild-type strain (FIG. 20). Contrary to TCA cycle intermediates, asparagine supplementation inhibited carotenogenesis in a hyper producing strain of X. dendrorhous impaired to assimilate nitrogen sources (An, 2001). This suggests that asparagine overproduction can be detrimental for AX production and potentially can be used as a metabolic strategy to improve AX production. Also, asparagine abundance is probably associated with a decrease in the Carbon/Nitrogen ratio which has been associated with a decrease in carotenoid production in X. dendrorhous (Fomtana et al., 1996). Overall, these metabolic differential analyses suggest a correlation between genome changes in the BPAX-A1 mutant strain and its transcriptome and metabolome profile, which potentially increased AX production (FIGS. 14-17 and Table 9). The main driver for the improved AX production in the mutant BPAX-A1 strain seems likely to be a positive regulation of its respiratory electron transport chain (FIG. 20). This improved the availability of electrons required the incorporation of oxygen groups to the AX molecule and the increase the oxidative stress through ROS, which has been suggested to be a trigger of AX production in X. dendrorhous (Liu and Wu, 2006).

### Oxidative Stress Response in X. Dendrorhous

Carotenoids, including AX, present antioxidant properties that have been associated with survival mechanisms in X. dendrorhous and other microorganisms (Schroeder and Johnson, 1995). Normally, ROS are generated by an overflow of electrons in the electron respiratory chain triggered by an imbalance of electrons transfer during the reduction and oxidation of the ubiquinone pool. AX can quench ROS analogously to the superoxide dismutase. In this example, it was observed that the mutant strain upregulated genes associated with the electron transport chain that in turn triggered mechanisms associated with protection against ROS. First, the AX and total carotenoids accumulation in the mutant strain was significantly higher (p<05) than the ones found in the wild-type strain across all the growth and production phases (FIGS. 14-17; FIG. 20; Table 9).

Second, in yeast, hydrogen peroxide is increased during p-oxidation of lipids in the peroxisome, which can be neutralized by catalases (Yin et al., 2009). It has been reported that X. dendrorhous shows low activity of this enzyme (Schroeder and Johnson, 1995). Here, at the transcriptome level, two catalases genes were significantly upregulated in the mutant strain: the CDZ96425 (Log 2 2.89, q<0.05) and CDZ98863 (Log 2 1.91, q<0.05) genes, respectively. This suggests that the mutant X. dendrorhous strain may use catalases as a mechanism to alleviate oxidative stresses. And third, a report suggests that X. dendrorhous uses an antioxidant core which is modulated by the carotenoid production including monooxygenases, a cytochrome P450 enzyme, phosphoglucomutase, and glyceraldehyde 3-phosphate (Flores-Cotera et al., 2001; Martinez-Moya et al., 2015).

It is reported that X. dendrorhous showed low levels of superoxide dismutase, or glutathione peroxidase enzymes under inductions with single oxygen and peroxyl radicals (Schroeder and Johnson, 1995). However, in a separate study, at the proteome level, it was identified activity of the superoxide dismutase and some glutathione enzymes when grown on succinate as a carbon source, which increased AX by 2.33-fold (Martinez-Moya et al., 2015). In agreement with that study, here two glutathione-S-transferase genes were significantly upregulated in the mutant strain: CED84930 (Log 2 6.09, q<0.05) and CDZ97957 (Log 2 5.72, q<0.05) (Table 16 and Table 17). It is reported that the glutathione S-transferases counteract the mutagenic effect of aldehyde products of lipid peroxidation (Ames et al., 1993). Generally, glutathione, glutathione enzymes, and superoxide dismutase are used as oxidative stress biomarkers in trials to evaluated the antioxidant effect of certain chemicals or drugs (Ames et al., 1993). Using these markers, AX has shown to be effective to stimulate these oxidative stress mechanisms in rainbow trout (Elia et al., 2019). In addition, the highest overexpressed gene in the mutant strain was the conidiation-specific protein 6 gene (CED85080) (Log 2 9.29, q<0.05). This protein has been associated with survival mechanisms against environmental stresses (Zhang et al., 2016). Overall, the mutant strain showed upregulation of the electron transport chain which might increased ROS triggering some protective oxidative stress mechanisms including higher AX production, upregulation of AX synthase, P450 reductase, and glutathione-related genes.

### Lipid Biosynthesis

The fatty acid pathway in X. dendrorhous has been elucidated (Sharma et al., 2015). The synthesis starts with the acetyl CoA carboxylase and the acyl carrier protein (ACP) for the formation of acetyl-ACP and malonyl-ACP, which is followed by condensations, ketoacyl reduction, a dehydratase reaction, and enoyl reduction all the way to palmityl-CoA are catalyzed by two multi-enzyme complexes FAS1 and FAS2. The FAS1 has functional domains of malony transferase, enoly reductase, hydroxyacyl dehydratase, and malony/palmitoyl transferase and FAS2 has functional groups of ketoacyl reductase, ketoacyl synthase, and phosphopantetheinyl transferase. The additional genes involved in the elongation of C16 to C18 fatty acid by the fatty acid elongase, and the insertion of a delta-9 and a delta-12 double bond by delta 9 fatty acid desaturase, and delta 12 fatty acid desaturase, respectively. From the mitochondrial fatty acid enzymes, palmitic acid can be synthesised by palmitoyl thioesterase from palmytil-ACP. Condensation, ketoacyl reduction, dehydratase, and enoyl-reduction can be also carried out by acetyl-CoA acyltransferase, 2 ketoacyl reductase, enoyl-CoA hydratase, or 3-hydroxyacyl CoA dehydrogenase, respectively.

In this example, another mutation was found in the delta 9 fatty acid desaturase. A report suggest that this enzyme has preferences for substrates of C18:1 and C16:1 converting to C18:2 and C16:2 fatty acids (L. Zhang et al., 2020). The most abundant fatty acids in the P. rhodozyma pathway are linoleate (C18:2), stearic acid (18:0), oleic acid (C18:1), palmitic acid (C16:1), and hexadecanoic acid (16:2) (Sharma et al., 2015). Although not significant, the delta 9 fatty acid desaturase gene (CED83656) was nominally downregulated in the mutant strain (Log 2 −0.53, q>0.05). This suggests that delta 9 fatty acid desaturase might be influencing the fatty acid pathway in the novel strains of P. rhodozyma. A decrease in the fatty acid and ergosterol metabolism in P. rhodozyma increased AX production (Miao et al., 2011). Also, the FAS1 gene (CED83610) was significantly upregulated in the mutant strain (Log 2 1.92, q<0.05). Similarly, the delta-6-desaturase gene (CED83550) was significantly upregulated (Log 2 1.38, q<0.05). This gene is the first step in the degradation of linoleic acid before to be utilised by the microorganism (Horrobin et al., 1993). Overall, these findings provide a possible explanation as to why linoleic acid was more abundant in the wild-type strain than in the mutant strain at the end of the fermentation.

### Example 3: Proteomic Analysis of a High Astaxanthin Producer Strain of Xanthophyllomyces dendrorhous

In this example, proteomic analysis was performed to complement the metabolomic and transcriptomic analysis of Example 2.

### Materials and Methods

### Strains, Media, and Growth Conditions

Strains, media, and growth conditions were as for Example 2, above.

### Proteomic Extraction, Quantification, Digestion, and SWATH Analyses.

Samples equivalent to 20-50 A600 units were harvested from the instrumented fermenters at Growth Phase 3 and Growth Phase 4 as described in Example 2. Cells were pelleted at 14000×g for 5 min at 4° C. The supernatant was discarded, and the pellet was washed twice with ice-cold water. The washed pellet was then re-suspended in 2 mL of sodium dodecyl sulfate (SDS) lysis buffer and transferred to a tubes containing 0.5 g of acid washed glass beads (Sigma Cat. No. G1152-100G). The tubes were chilled on ice for 5 minutes and then disrupted using five bead-beating cycles of 5 min using a Qiagen Tissue Lyser II. After disruption, the tubes were centrifuged at 14000×g for 10 min at 4° C.

The extracted proteins were quantified using the Pierce BCA Protein Assay Kit (ThermoFisher Cat. No. 23225). The FASP and C18 StageTip protocols were then applied to digest the extracted proteins and desalt the peptides, respectively (Jacek R Wisniewski, Alexandre Zougman, Nagarjuna Nagaraj, 2009). The peptides of digested samples were quantified using the Pierce Quantitative Colorimetric Peptide Kit (ThermoFisher Cat. No. 23275) and used to normalize their concentration before submitting the samples to mass spectrometry (MS) analysis. The sequential window acquisition of all theoretical mass spectra (SWATH) technique was then applied to the peptide samples using the Sciex 5600 QTOF (Yeo et al., 2016).

To prepare the library, a mixture of all peptide samples (PBQC) was MS analysed in Data Dependent Acquisition mode (DDA). For this, ProteinPilot 5.0.2 was first used to identify proteins using the Paragon method and the published and annotated P. rhodozyma CBS6938 genome using a quality threshold of 1.3 (95%) (Sharma et al., 2015). Skyline was then used to create the spectral library and SWATH mode analysed samples. Differential proteome expression was assessed between the mutant X. dendrorhous BPAX-A1 (referred to as MYM0 in Example 1) and wild-type X. dendrorhous CBS 6938 at Phase 3 and Phase 4.

### Results

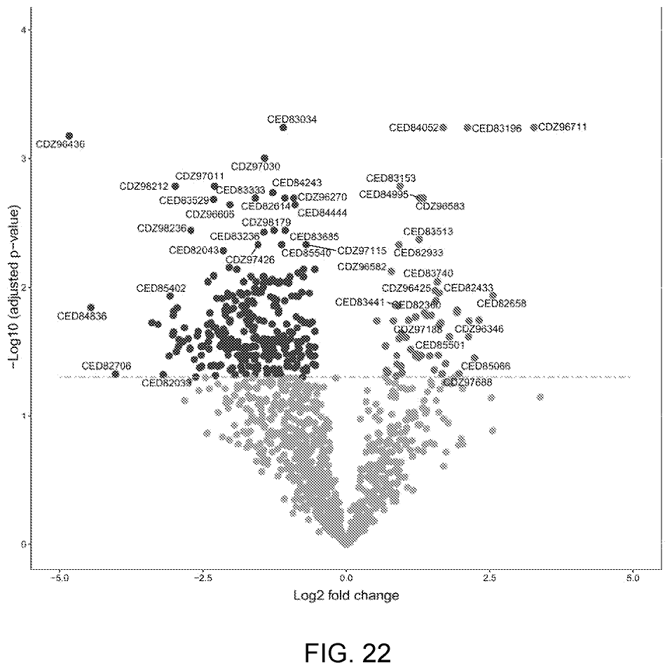

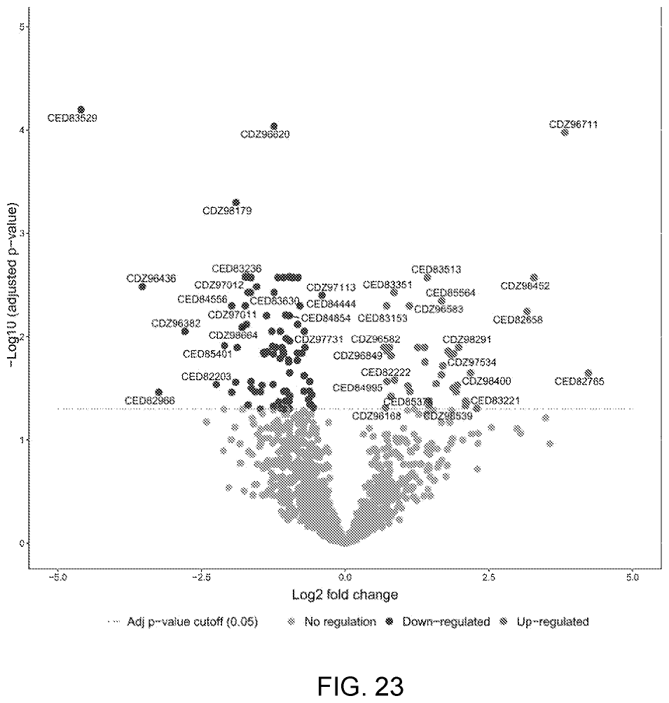

Differential protein expression results are set out for Growth Phase 3 in FIG. 22 and Table 18; and for Growth Phase 4 in FIG. 23 and Table 19.

### Example 4: Products Incorporating Astaxanthin

This example provides details of certain typical formulation for supplementing domestic or companion animals, the formulation including astaxanthin produced as herein described. The formulation may be referred to as ‘NatuAX’, although without limitation thereto.

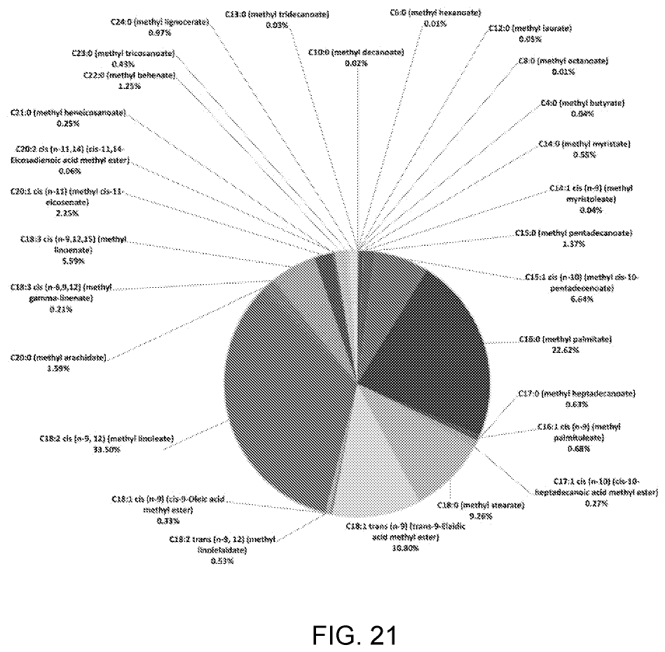

The formulations are also rich in dietary fibre, vitamins, beta-glucan, and amino acids. An analysis of fatty acid profile for the formulations is provided in FIG. 21. An analysis of amino acid profile is provided in Table 11.

Formulations for feline, canine, and equine use have been developed. All formulations include astaxanthin and beta-glucans.

In general, astaxanthin may provide benefits including:
Antioxidant activity for supporting the immune system
Mitochondrial protection against reactive oxygen species
Reduction in cellular damage and lipid peroxidation
Improve response and processing of antigens

In general, beta-glucans may provide benefits including:
Stimulation of the immune system
Reduction of blood concentration of total cholesterol
Antioxidant properties
Reduction of inflammatory response

Equine variations comprise polyphenols, which may provide benefits including:
Combat inflammation and the natural aging process (‘inflamm-aging’)
Decrease the amount of non-steroidal anti-inflammatory drugs administered to older horses
Antioxidant activity for reducing oxidative stress and improve immune function
Support muscle recovery in endurance horses
   Specifications for the feline, canine, and equine formulations are as follows:

### Feline

### Component Percentages:

Astaxanthin: 0.25%=2.5 g/kg
beta-Glucan: 15%=150 g/kg
Carriers: =847.5% g/kg=847.5 g/kg

### Minimum and Maximum Recommended Dose Rates:

Adult cats<5 kg: 1 g/day
Adult cats>5 kg: 2 g/day
Kittens and geriatric cats: 2 g/day

### Canine

### Component Percentages:

Astaxanthin: 0.25%=2.5 g/kg
beta-Glucan: 15%=150 g/kg
Carriers: =847.5% g/kg=847.5 g/kg

### Minimum and Maximum Recommended Dose Rates:

Extra small dogs (8 kg)=1 g/day
Small dogs (16 kg): 2 g/day
Medium dogs (24 kg): 3 g/day
Large dogs (32 kg): 4 g/day
Extra large dogs (40 kg): 5 g/day

### (Puppies and Geriatric Dogs Require Double the Recommended Dose)

### Equine

### Component Percentages:

Astaxanthin: 0.075%=0.75 g/kg
beta-Glucan: 4.22%=42.2 g/kg
Polyphenols: 2.5%=25 g/kg
Carriers: 93.21% g/kg=932.05 g/kg

### Minimum and Maximum Recommended Dose Rates:

100 g per day orally or mixed in feed

(Horses in poor condition or commencing training require double the recommended dose for the first 3 weeks)

### REFERENCES

Adams, K. J., Pratt, B., Bose, N., Dubois, L. G., St. John-Williams, L., Perrott, K. M., Ky, K., Kapahi, P., Sharma, V., Maccoss, M. J., Moseley, M. A., Colton, C. A., Maclean, B. X., Schilling, B., Thompson, J. W., 2020. Skyline for Small Molecules: A Unifying Software Package for Quantitative Metabolomics. J. Proteome Res. 19, 1447-1458. doi.org/10.1021/acs.jproteome.9b00640
Ahn, S., Jung, J., Jang, I. A., Madsen, E. L., Park, W., 2016. Role of glyoxylate shunt in oxidative stress response. J. Biol. Chem. 291, 11928-11938. doi.org/10.1074/jbc.M115.708149
Alcaíno, J., Barahona, S., Carmona, M., Lozano, C., Marcoleta, A., Niklitschek, M., Sepúlveda, D., Baeza, M., Cifuentes, V., 2008a. Cloning of the cytochrome p450 reductase (crtR) gene and its involvement in the astaxanthin biosynthesis of Xanthophyllomyces dendrorhous. BMC Microbiol. 8, 1-13. doi.org/10.1186/1471-2180-8-169
Alcaíno, J., Barahona, S., Carmona, M., Lozano, C., Marcoleta, A., Niklitschek, M., Sepúlveda, D., Baeza, M., Cifuentes, V., 2008b. Cloning of the cytochrome p450 reductase (crtR) gene and its involvement in the astaxanthin biosynthesis of Xanthophyllomyces dendrorhous. BMC Microbiol. 8. doi.org/10.1186/1471-2180-8-169
Ambati, R. R., Moi, P. S., Ravi, S., Aswathanarayana, R. G., 2014. Astaxanthin: Sources, extraction, stability, biological activities and its commercial applications—A review. Mar. Drugs 12, 128-152. doi.org/10.3390/md12010128
Ames, B. N., Shigenaga, M. K., Hagen, T. M., 1993. Oxidants, antioxidants, and the degenerative diseases of aging (cancer/mutation/endogenous DNA adducts/oxygen radicals), Proc. Natl. Acad. Sci. USA.
An, G. H., 2001. Improved growth of the red yeast, Phaffia rhodozyma (Xanthophyllomyces dendrorhous), in the presence of tricarboxylic acid cycle intermediates. Biotechnol. Lett. 23, 1005-1009. doi.org/10.1023/A:1010523800650
An, G. H., Jang, B. G., Cho, M. H., 2001. Cultivation of the carotenoid-hyperproducing mutant 2A2N of the red yeast Xanthophyllomyces dendrorhous (Phaffia rhodozyma) with molasses. J. Biosci. Bioeng. 92, 121-5. doi.org/10.1263/jbb.92.121
An, G. H., Schuman, D. B., Johnson, E. A., 1989. Isolation of Phaffia rhodozyma Mutants with Increased Astaxanthin Content. Appl. Environ. Microbiol. 55, 116-24.
Andrews, S., 2010. FastQC: A quality control tool for high throughput sequence data [WWW Document]. Online. URL www.bioinformatics.babraham.ac.uk/projects/fastqc/
Auch, A. F., Klenk, H.-P., Göker, M., 2010. Standard operating procedure for calculating genome-to-genome distances based on high-scoring segment pairs. Stand. Genomic Sci. 2, 142-8. doi.org/10.4056/sigs.541628
Auwera, G. A., Carneiro, M. O., Hartl, C., Poplin, R., del Angel, G., Levy-Moonshine, A., Jordan, T., Shakir, K., Roazen, D., Thibault, J., Banks, E., Garimella, K. V., Altshuler, D., Gabriel, S., DePristo, M. A., 2013. From FastQ Data to High-Confidence Variant Calls: The Genome Analysis Toolkit Best Practices Pipeline. Curr. Protoc. Bioinforma. 43, 11.10.1-11.10.33. doi.org/10.1002/0471250953.bi1110s43
Bergstrom, J. R., Nelssen, J. L., Tokach, M. D., Bergstrom, J. R.; Nelssen, J. L.; Tokach, M. D.; Derouchey, J. M.; Goodband, R. D.; Dritz, S. S., 1968. An evaluation of astaxanthin as a nutraceutical growth promoter An evaluation of astaxanthin as a nutraceutical growth promoter in starter diets for weanling pigs in starter diets for weanling pigs Recommended Citation Recommended Citation. Kansas Agric. Exp. Stn. Res. Reports 0. doi.org/10.4148/2378-5977.6963
Bolger, A. M., Lohse, M., Usadel, B., 2014. Trimmomatic: A flexible trimmer for Illumina sequence data. Bioinformatics 30, 2114-2120. doi.org/10.1093/bioinformatics/btu170
Bon, J. A., Leathers, T. D., Jayaswal, R. K., 1997. Isolation of astaxanthin-overproducing mutants of Phaffia rhodozyma. Biotechnol. Lett. 19, 109-112. doi.org/10.1023/A:1018391726206
Brühl, W. K., Hassloch, K. H., Neuhofen, J. P., Speyer, H. E., 1997. Preparation of astaxanthin.
Calo, P., de Miguel, T., Velazquez, J. B., Villa, T. G., 1995. Mevalonic acid increases trans-astaxanthin and carotenoid biosynthesis in Phaffia rhodozyma. Biotechnol. Lett. 17, 575-578.
Canelas, A. B., Ras, C., ten Pierick, A., van Dam, J. C., Heijnen, J. J., van Gulik, W. M., 2008. Leakage-free rapid quenching technique for yeast metabolomics. Metabolomics 4, 226-239. doi.org/10.1007/s11306-008-0116-4
Canelas, A. B., Ten Pierick, A., Ras, C., Seifar, R. M., Van Dam, J. C., Van Gulik, W. M., Heijnen, J. J., 2009. Quantitative evaluation of intracellular metabolite extraction techniques for yeast metabolomics. Anal. Chem. 81, 7379-7389. doi.org/10.1021/ac900999t
Cannizzaro, C., Christensen, B., Nielsen, J., Stockar, U. Von, 2004. Metabolic network analysis on Phaffia rhodozyma yeast using 13C-labeled glucose and gas chromatography-mass spectrometry. Metab. Eng. 6, 340-351. doi.org/10.1016/j.ymben.2004.06.001
Capelli, B., Bagchi, D., Cysewski, G. R., 2014. Synthetic astaxanthin is significantly inferior to algal-based astaxanthin as an antioxidant and may not be suitable as a human nutraceutical supplement. Nutrafoods 2013 124 12, 145-152. doi.org/10.1007/S13749-013-0051-5
Capelli, B., Talbott, S., Ding, L., 2019. Astaxanthin sources: Suitability for human health and nutrition. Funct. Foods Heal. Dis. 9, 430-445. doi.org/10.31989/ffhd.v9i6.584
Chen, T., Speight, R., Sadowsky, P., Blanksby, S., 2020. COMPOSITIONAL ANALYSIS OF NEW MICROBIAL FEED SUPPLEMENTS PRODUCED FROM MEAT PROCESSING CO-PRODUCTS. Queensland University of Technology.
Chumpolkulwong, N., Kakizono, T., Nagai, S., Nishio, N., 1997. Increased astaxanthin production by Phaffia rhodozyma mutants isolated as resistant to diphenylamine. J. Ferment. Bioeng. 83, 429-434. doi.org/10.1016/S0922-338X(97)82996-0
Chun, S. B., Chin, J. E., Bai, S., An, G. H., 1992. Strain improvement of Phaffia rhodozyma by protoplast fusion. FEMS Microbiol. Lett. 93, 221-226. doi.org/10.1016/0378-1097(92)90466-2
Cingolani, P., Platts, A., Wang, L. L., Coon, M., Nguyen, T., Wang, L., Land, S. J., Lu, X., Ruden, D. M., 2012. A program for annotating and predicting the effects of single nucleotide polymorphisms, SnpEff: SNPs in the genome of Drosophila melanogaster strain w 1118; iso-2; iso-3. Fly (Austin). 6, 80-92. doi.org/10.4161/fly.19695
Cruz, J. M., Parajo, J. C., 1998. Improved astaxanthin production by Xanthophyllomyces dendrorhous growing on enzymatic wood hydrolysates containing glucose and cellobiose. Food Chem. 63, 479-484. doi.org/10.1016/S0308-8146(98)00061-2
De Livera, A. M., Dias, D. A., De Souza, D., Rupasinghe, T., Pyke, J., Tull, D., Roessner, U., McConville, M., Speed, T. P., 2012. Normalizing and integrating metabolomics data. Anal. Chem. 84, 10768-10776. doi.org/10.1021/ac302748b
Dominguez-Bocanegra, A. R., Torres-Munoz, J. A., 2004. Astaxanthin hyperproduction by Phaffia rhodozyma (now Xanthophyllomyces dendrorhous) with raw coconut milk as sole source of energy. Appl. Microbiol. Biotechnol. 66, 249-252. doi.org/10.1007/s00253-004-1686-3
Elia, A. C., Prearo, M., Josef, A., Dorr, M., Pacini, N., Magara, G., Brizio, P., Gasco, L., Abete, M. C., 2019. Journal of Toxicology and Environmental Health, Part A Current Issues Effects of astaxanthin and canthaxanthin on oxidative stress biomarkers in rainbow trout Effects of astaxanthin and canthaxanthin on oxidative stress biomarkers in rainbow trout. J. Toxicol. Environ. Heal. Part A 82, 760-768. doi.org/10.1080/15287394.2019.1648346
Flores-Cotera, L. B., Chávez-Cabrera, C., Martinez-Cárdenas, A., Sánchez, S., García-Flores, O. U., 2021. Deciphering the mechanism by which the yeast Phaffia rhodozyma responds adaptively to environmental, nutritional, and genetic cues. J. Ind. Microbiol. Biotechnol. 48. doi.org/10.1093/jimb/kuab048
Flores-Cotera, L. B., Martin, R., Sánchez, S., 2001. Citrate, a possible precursor of astaxanthin in Phaffia rhodozyma: Influence of varying levels of ammonium, phosphate and citrate in a chemically defined medium. Appl. Microbiol. Biotechnol. 55, 341-347. doi.org/10.1007/s002530000498
Flores-Cotera, L. B., Sánchez, S., 2001. Copper but not iron limitation increases astaxanthin production by Phaffia rhodozyma in a chemically defined medium. Biotechnol. Lett. 23, 793-797. doi.org/10.1023/A:1010358517806
Fomtana, J. D., Guimarães, M. F., Martins, N. T., Fontana, C. A., Baron, M., 1996. Culture of the astaxanthinogenic yeast Phaffia rhodozyma in low-cost media. Appl. Biochem. Biotechnol. 57-58, 413-422. doi.org/10.1007/BF02941721
Fontana, J. D., Chocial, M. B., Baron, M., Guimaraes, M. F., Maraschin, M., Ulhoa, C., Florêncio, J. a, Bonfim, T. M., 1997. Astaxanthinogenesis in the yeast Phaffia rhodozyma: optimization of low-cost culture media and yeast cell-wall lysis. Appl. Biochem. Biotechnol. 63-65, 305-14. doi.org/10.1007/BF02920432
Fontana, J. D., Czeczuga, B., Bonfim, T. M. B., Chociai, M. B., Oliveira, B. H., Guimarães, M. F., Baron, M., 1996. Bioproduction of carotenoids: The comparative use of raw sugarcane juice and depolymerized bagasse by Phaffia rhodozyma. Bioresour. Technol. 58, 121-125. doi.org/10.1016/S0960-8524(96)00092-2
Fu, Y. H., Marzluf, G. A., 1990. nit-2, the major nitrogen regulatory gene of Neurospora crassa, encodes a protein with a putative zinc finger DNA-binding domain. Mol. Cell. Biol. 10, 1056-1065. doi.org/10.1128/mcb.10.3.1056
Gassel, S., Schewe, H., Schmidt, I., Schrader, J., Sandmann, G., 2013. Multiple improvement of astaxanthin biosynthesis in Xanthophyllomyces dendrorhous by a combination of conventional mutagenesis and metabolic pathway engineering. Biotechnol. Lett. 35, 565-569. doi.org/10.1007/s10529-012-1103-4
Gil-Hwan, A. N., Schuman, D. B., Johnson, E. A., 1989. Isolation of Phaffia rhodozyma mutants with increased astaxanthin content. Appl. Environ. Microbiol. 55, 116-124. doi.org/10.1128/aem.55.1.116-124.1989
Goldstein, J. L., Brown, M. S., 1990a. Regulation of the mevalonate pathway. Nature 343, 425-430. doi.org/10.1038/343425a0
Goldstein, J. L., Brown, M. S., 1990b. Regulation of the mevalonate pathway. Nature. doi.org/10.1038/343425a0
Grinberg, A. V., Hannemann, F., Schiffler, B., Miller, J., Heinemann, U., Bernhardt, R., 2000. Adrenodoxin: Structure, stability, and electron transfer properties. Proteins Struct. Funct. Genet. 40, 590-612. doi.org/10.1002/1097-0134(20000901)40:4<590::AID-PROT50>3.0.CO;2-P
Gu, W. L., An, G. H., Johnson, E. a, 1997. Ethanol increases carotenoid production in Phaffia rhodozyma. J. Ind. Microbiol. Biotechnol. 19, 114-117. doi.org/10.1038/sj.jim.2900425
Haard, N. F., 1988. Astaxanthin formation by the yeast Phaffia rhodozyma on molases. Biotechnol. Lett. 10, 609-614.
Hanukoglu, I., 1992. Steroidogenic enzymes: Structure, function, and role in regulation of steroid hormone biosynthesis. J. Steroid Biochem. Mol. Biol. 43, 779-804. doi.org/10.1016/0960-0760(92)90307-5
Hara, K. Y., Morita, T., Mochizuki, M., Yamamoto, K., Ogino, C., Araki, M., Kondo, A., 2014. Development of a multi-gene expression system in Xanthophyllomyces dendrorhous. Microb. Cell Fact. 13, 175. doi.org/10.1186/s12934-014-0175-3
Hasan, M., Azhar, M., Nangia, H., Bhatt, P. C., Panda, B. P., 2016. Influence of High Pressure Homogenization, Ultra-Sonication and Supercritical Fluid on Free Astaxanthin Extraction From β-Glucanase Treated Phaffia rhodozyma Cells. Prep. Biochem. Biotechnol. 0, 150527100841001. doi.org/10.1080/10826068.2014.995807
Higashida, K., Nakashimada, Y., Kakizono, T., Nishio, N., 1997. Influence of Oxygen and Glucose on Primary Metabolism and Astaxanthin Production by Phaffia rhodozyma in Batch and Fed-Batch Cultures: Kinetic and Stoichiometric Analysis. Microbiology 63, 4471-4478.
Higuera-Ciapara, I., Félix-Valenzuela, L., Goycoolea, F. M., 2006. Astaxanthin: a review of its chemistry and applications. Crit. Rev. Food Sci. Nutr. 46, 185-96. doi.org/10.1080/10408690590957188
Honda, T., Takahashi, J., Aichi-Ken, J., Toyama-ken, J., 2007. ASTAXANTHIN-CONTAINING PET FOODS. US 2007/0128310 A1.
Horrobin, D. F., Crawford, M. A., Vergroesen, A. J., Horrobin, D. F., 1993. Fatty acid metabolism in health and disease: The role of Δ-6-desaturase, in: American Journal of Clinical Nutrition. Am J Clin Nutr. doi.org/10.1093/ajcn/57.5.732S
Jacek R Wisniewski, Alexandre Zougman, Nagarjuna Nagaraj, M. M. (2009) Universal sample preparation method for proteome analysis. Nat Methods 6: 377-362.
Jian, X., Boerwinkle, E., Liu, X., 2013. In silico tools for splicing defect prediction: a survey from the viewpoint of end users. doi.org/10.1038/gim.2013.176
Johnson, E. a., An, G.-H., 1991. Astaxanthin from Microbial Sources. Crit. Rev. Biotechnol. 11, 297-326. doi.org/10.3109/07388559109040622
Kajiwara, S., Fraser, P. D., Kondo, K., Misawa, N., 1997. Expression of an exogenous isopentenyl diphosphate isomerase gene enhances isoprenoid biosynthesis in Escherichia coli. Biochem. J. 324, 421-426. doi.org/10.1042/bj3240421
Krogh, A., Larsson, B., Von Heijne, G., Sonnhammer, E. L. L., 2001. Predicting transmembrane protein topology with a hidden Markov model: Application to complete genomes. J. Mol. Biol. 305, 567-580. doi.org/10.1006/jmbi.2000.4315
Kryvokhyzha, D., 2016. GATK the best practice for a non-model organism [WWW Document]. URL evodify.com/gatk-in-non-model-organism/
Langmead, B., Salzberg, S. L., 2012. Fast gapped-read alignment with Bowtie 2. Nat. Methods 9, 357-9. doi.org/10.1038/nmeth.1923
Lengger, B., Jensen, M. K., 2020. Engineering G protein-coupled receptor signalling in yeast for biotechnological and medical purposes. FEMS Yeast Res. doi.org/10.1093/femsyr/foz087
Lewis, M. J., Ragot, N., Berlant, M. C., Miranda, M., 1990. Selection of astaxanthin-overproducing mutants of Phaffia rhodozyma with B-ionone. Appl. Environ. Microbiol. 56, 2944-2945.
Li, H., Durbin, R., 2010. Fast and accurate long-read alignment with Burrows-Wheeler transform. Bioinformatics 26, 589-595. doi.org/10.1093/bioinfornatics/btp698
Li, H., Handsaker, B., Wysoker, A., Fennell, T., Ruan, J., Homer, N., Marth, G., Abecasis, G., Durbin, R., 2009. The Sequence Alignment/Map format and SAMtools. Bioinformatics 25, 2078-9. doi.org/10.1093/bioinformatics/btp352
Li, X., Wang, X., Duan, C., Yi, S., Gao, Z., Xiao, C., Agathos, S. N., Wang, G., Li, J., 2020. Biotechnological production of astaxanthin from the microalga Haematococcus pluvialis. Biotechnol. Adv. 43, 107602. doi.org/10.1016/J.BIOTECHADV.2020.107602
Libkind, D., Moline, M., Van Broock, M., 2011. Production of the UVB-absorbing compound mycosporine-glutaminol-glucoside by Xanthophyllomyces dendrorhous (Phaffia rhodozyma). FEMS Yeast Res. 11, 52-59. doi.org/10.1111/j.1567-1364.2010.00688.x
Liu, X., Osawa, T., 2007. Cis astaxanthin and especially 9-cis astaxanthin exhibits a higher antioxidant activity in vitro compared to the all-trans isomer. Biochem. Biophys. Res. Commun. 357, 187-193. doi.org/10.1016/J.BBRC.2007.03.120
Liu, Y. S., Wu, J. Y., 2006. Hydrogen peroxide-induced astaxanthin biosynthesis and catalase activity in Xanthophyllomyces dendrorhous. Appl. Microbiol. Biotechnol. 73, 663-668. doi.org/10.1007/s00253-006-0501-8
Luna-Flores, C. H., Palfreyman, R. W., Krömer, J. O., Nielsen, L. K., Marcellin, E., 2016. Improved production of propionic acid using genome shuffling. Biotechnol. J. 1-12. doi.org/10.1002/biot.201600120
Luna-Flores, C. H., Ramirez-Cordova, J. J., Pelayo-Ortiz, C., Femat, R., Herrera-López, E. J., 2010. Batch and fed-batch modeling of carotenoids production by Xanthophyllomyces dendrorhous using Yucca fillifera date juice as substrate. Biochem. Eng. J. 53, 131-136. doi.org/10.1016/j.bej.2010.10.004
Luna-Flores, C. H., Stowers, C. C., Cox, B. M., Nielsen, L. K., Marcellin, E., 2018. Linking genotype and phenotype in an economically viable propionic acid biosynthesis process. Biotechnol. Biofuels 11, 224. doi.org/10.1186/s13068-018-1222-9
Luna-Flores, C. H., Stowers, C. C., Cox, B. M., Nielsen, L. K., Marcellin, E., 2017. Scalable and economical process for propionic acid biosynthesis. Biotechnol. Biofuels Submitted.
Martinez-Cárdenas, A., Chávez-Cabrera, C., Vasquez-Bahena, J. M., Flores-Cotera, L. B., 2018. A common mechanism explains the induction of aerobic fermentation and adaptive antioxidant response in Phaffia rhodozyma. Microb. Cell Fact. 17, 53. doi.org/10.1186/s12934-018-0898-7
Martinez-Moya, P., Niehaus, K., Alcaino, J., Baeza, M., Cifuentes, V., 2015. Proteomic and metabolomic analysis of the carotenogenic yeast Xanthophyllomyces dendrorhous using different carbon sources. BMC Genomics 16, 1-18. doi.org/10.1186/s12864-015-1484-6
Mendez, K. M., Pritchard, L., Reinke, S. N., Broadhurst, D. I., 2019. Toward collaborative open data science in metabolomics using Jupyter Notebooks and cloud computing. Metabolomics 15, 1-16. doi.org/10.1007/sI1306-019-1588-0
Miao, L., Chi, S., Tang, Y., Su, Z., Yin, T., Guan, G., Li, Y., 2011. Astaxanthin biosynthesis is enhanced by high carotenogenic gene expression and decrease of fatty acids and ergosterol in a Phaffia rhodozyma mutant strain. FEMS Yeast Res. 11, 192-201. doi.org/10.1 111/j.1567-1364.2010.00705.x
Miller, G. L., 1959. Use of Dinitrosalicylic Acid Reagent for Determination of Reducing Sugar. Anal. Chem. 31, 426-428. doi.org/10.1021/ac60147a030
Misawa, N., 2021. Carotenoids: Biosynthetic and Biofunctional Approaches.
Moretti, V. M., Mentasti, T., Bellagamba, F., Luzzana, U., Caprino, F., Turchini, G. M., Giani, I., Valfrè, F., 2006. Determination of astaxanthin stereoisomers and colour attributes in flesh of rainbow trout (Oncorhynchus mykiss) as a tool to distinguish the dietary pigmentation source. Food Addit. Contain. 23, 1056-63. doi.org/10.1080/02652030600838399
Mussagy, C. U., Pereira, J. F. B., Dufossé, L., Raghavan, V., Santos-Ebinuma, V. C., Pessoa, A., 2021. Advances and trends in biotechnological production of natural astaxanthin by Phaffia rhodozyma yeast. Crit. Rev. Food Sci. Nutr. 0, 1-15. doi.org/10.1080/10408398.2021.1968788
Najafi, N., Ahmadi, A.-R., Hosseini, R., Golkhoo, S., 2011. Gamma irradiation as a useful tool for the isolation of astaxanthin-overproducing mutant strains of Phaffia rhodozyma. Can. J. Microbiol. 57, 730-734. doi.org/10.1139/W11-060
Ni, H., Chen, Q., Ruan, H., Yang, Y., Li, L., Wu, G., Hu, Y., He, G., 2007. Studies on optimization of nitrogen sources for astaxanthin production by Phaffia rhodozyma. J. Zhejiang Univ. Sci. B 8, 365-70. doi.org/10.1631/jzus.2007.B0365
Niklitschek, M., Alcaino, J., Barahona, S., Sepúlveda, D., Lozano, C., Carmona, M., Marcoleta, A., Martinez, C., Lodato, P., Baeza, M., Cifuentes, V., 2008. Genomic organization of the structural genes controlling the astaxanthin biosynthesis pathway of Xanthophyllomyces dendrorhous. Biol. Res. 41, 93-108. doi.org/10.4067/S0716-97602008000100011
Ojima, K., Breitenbach, J., Visser, H., Setoguchi, Y., Tabata, K., Hoshino, T., Van Den Berg, J., Sandmann, G., 2006. Cloning of the astaxanthin synthase gene from Xanthophyllomyces dendrorhous (Phaffia rhodozyma) and its assignment as a β-carotene 3-hydroxylase/4-ketolase. Mol. Genet. Genomics 275, 148-158. doi.org/10.1007/s00438-005-0072-x
Pan, X., Wang, B., Duan, R., Jia, J., Li, J., Xiong, W., Ling, X., Chen, C., Huang, X., Zhang, G., Lu, Y., 2020. Enhancing astaxanthin accumulation in Xanthophyllomyces dendrorhous by a phytohormone: metabolomic and gene expression profiles. Microb. Biotechnol. doi.org/10.1111/1751-7915.13567
Park, J. S., Kim, H. W., Mathison, B. D., Hayek, M. G., Massimino, S., Reinhart, G. A., Chew, B. P., 2010. Astaxanthin uptake in domestic dogs and cats. Nutr. Metab. 7, 52. doi.org/10.1186/1743-7075-7-52
Perenlei, G., Tojo, H., Okada, T., Kubota, M., Kadowaki, M., Fujimura, S., 2014. Effect of dietary astaxanthin rich yeast, Phaffia rhodozyma, on meat quality of broiler chickens. Anim. Sci. J. 85, 895-903. doi.org/10.1111/asj.12221
Reynders, M. B., Rawlings, D. E., Harrison, S. T. L., 1997. Demonstration of the Crabtree effect in Phaffia rhodozyma during continuous and fed-batch cultivation. Biotechnol. Lett. 19, 549-552. doi.org/10.1023/A:1018341421122
Rodriguez-Sáiz, M., De La Fuente, J. L., Barredo, J. L., 2010. Xanthophyllomyces dendrorhous for the industrial production of astaxanthin. Appl. Microbiol. Biotechnol. 88, 645-658. doi.org/10.1007/s00253-010-2814-x
Rohmer, M., 1999. The discovery of a mevalonate-independent pathway for isoprenoid biosynthesis in bacteria, algae and higher plants. Nat. Prod. Rep. 16, 565-574. doi.org/10.1039/a709175c
Sandmann, G., 1994. Carotenoid biosynthesis in microorganisms and plants. Eur. J. Biochem. 223, 7-24. doi.org/10.1111/j.1432-1033.1994.tb18961.x
Schewe, H., Kreutzer, A., Schmidt, I., Schubert, C., Schrader, J., 2017. High concentrations of biotechnologically produced astaxanthin by lowering pH in a Phaffia rhodozyma bioprocess. Biotechnol. Bioprocess Eng. 22, 319-326. doi.org/10.1007/s12257-016-0349-4
Schmidt, H., Jirstrand, M., 2006. Systems Biology Toolbox for MATLAB: A computational platform for research in systems biology. Bioinformatics 22, 514-515. doi.org/10.1093/bioinformatics/bti799
Schmidt, I., Schewe, H., Gassel, S., Jin, C., Buckingham, J., Hümbelin, M., Sandmann, G., Schrader, J., 2011. Biotechnological production of astaxanthin with Phaffia rhodozyma/Xanthophyllomyces dendrorhous. Appl. Microbiol. Biotechnol. doi.org/10.1007/s00253-010-2976-6
Schneider, R., Brors, B., Massow, M., Weiss, H., 1997. Mitochondrial fatty acid synthesis: A relic of endosymbiontic origin and a specialized means for respiration. FEBS Lett. 407, 249-252. doi.org/10.1016/S0014-5793(97)00360-8
Schroeder, W. a, Johnson, E. a, 1993. Antioxidant role of carotenoids in Phaffia rhodozyma. J. Gen. Microbiol. 139, 907-912. doi.org/10.1099/00221287-139-5-907
Schroeder, W. A., Johnson, E. A., 1995. Singlet oxygen and peroxyl radicals regulate carotenoid biosynthesis in Phaffia rhodozyma. J. Biol. Chem. 270, 18374-18379. doi.org/10.1074/jbc.270.31.18374
Sedmak, J. J., Weerasinghe, D. K., Jolly, S. O., 1990. Extraction and quantification of astaxanthin from Phaffia rhodozyma. Biotechnol. Tech. 4, 107-112.
Shah, M. M. R., Liang, Y., Cheng, J. J., Daroch, M., 2016. Astaxanthin-producing green microalga Haematococcus pluvialis: From single cell to high value commercial products. Front. Plant Sci. doi.org/10.3389/fpls.2016.00531
Sharma, R., Gassel, S., Steiger, S., Xia, X., Bauer, R., Sandmann, G., 2015. The genome of the basal agaricomycete Xanthophyllomyces dendrorhous provides insights into the organization of its acetyl-CoA derived pathways and the evolution of Agaricomycotina 1-13. doi.org/10.1186/s12864-015-1380-0
Silva, C. M., Borba, T. M., Burkert, C. A. V, Burkert, J. F. M., 2012. Carotenoid production by Phaffia rhodozyma using raw glycerol as an additional carbon source. Int. J. Food Eng. 8. doi.org/10.1515/1556-3758.2843
Srinivasan, S., Avadhani, N. G., 2012. Cytochrome c oxidase dysfunction in oxidative stress. Free Radic. Biol. Med. doi.org/10.1016/j.freeradbiomed.2012.07.021
Sun, N., Lee, S., Song, K. Bin, 2004. Characterization of a carotenoid-hyperproducing yeast mutant isolated by low-dose gamma irradiation. Int. J. Food Microbiol. 94, 263-267. doi.org/10.1016/50168-1605(03)00311-8
Thorvaldsdóttir, H., Robinson, J. T., Mesirov, J. P., 2013. Integrative Genomics Viewer (IGV): high-performance genomics data visualization and exploration. Brief Bioinform. 14, 178-92. doi.org/10.1093/bib/bbs017
Trapnell, C., Roberts, A., Goff, L., Pertea, G., Kim, D., Kelley, D. R., Pimentel, H., Salzberg, S. L., Rinn, J. L., Pachter, L., 2012. Differential gene and transcript expression analysis of RNA-seq experiments with TopHat and Cufflinks. Nat. Protoc. 7, 562-78. doi.org/10.1038/nprot.2012.016
Ukibe, K., Katsuragi, T., Tani, Y., Takagi, H., 2008. Efficient screening for astaxanthin-overproducing mutants of the yeast Xanthophyllomyces dendrorhous by flow cytometry. FEMS Microbiol. Lett. 286, 241-248. doi.org/10.1111/j.1574-6968.2008.01278.x
Verdoes, J. C., Krubasik, P., Sandmann, G., Van Ooyen, A. J. J., 1999. Isolation and functional characterisation of a novel type of carotenoid biosynthetic gene from Xanthophyllomyces dendrorhous. Mol. Gen. Genet. 262, 453-461. doi.org/10.1007/s004380051105
Xie, H., Zhou, Y., Hu, J., Chen, Y., Liang, J., 2014. Production of astaxanthin by a mutant strain of Phaffia rhodozyma and optimization of culture conditions using response surface methodology. Ann. Microbiol. 64, 1473-1481. doi.org/10.1007/s13213-013-0790-y
Yeo, K. Y. B., Chrysanthopoulos, P. K., Nouwens, A. S., Marcellin, E., and Schulz, B. L. (2016) High performance targeted mass spectrometry with precision data independent acquisition reveals site-specific glycosylation macroheterogeneity. Anal Biochem 510: 106-113.
Yin, Z., Stead, D., Walker, J., Selway, L., Smith, D. A., Brown, A. J. P., Quinn, J., 2009. A proteomic analysis of the salt, cadmium and peroxide stress responses in Candida albicans and the role of the HogI stress-activated MAPK in regulating the stress-induced proteome. Proteomics 9, 4686-4703. doi.org/10.1002/pmic.200800958
Zerbino, D. R., 2010. Using the Velvet de novo assembler for short-read sequencing technologies.

Curr. Protoc. Bioinformatics Chapter 11, Unit 11.5. doi.org/10.1002/0471250953.bi1105s31
Zhang, C., Chen, X., Too, H. P., 2020. Microbial astaxanthin biosynthesis: recent achievements, challenges, and commercialization outlook. Appl. Microbiol. Biotechnol. doi.org/10.1007/s00253-020-10648-2
Zhang, L., Chen, W., Yang, S., Zhang, Y., Xu, J., Yang, D., Wu, Z., Liu, T., Cao, J., 2020. Identification and Functional Characterization of a Novel Δ12 Fatty Acid Desaturase Gene from Haematococcus pluvialis. J. Ocean Univ. China 19, 1362-1370. doi.org/10.1007/s11802-020-4418-0
Zhang, Y., Navarro, E., Cánovas-Márquez, J. T., Almagro, L., Chen, H., Chen, Y. Q., Zhang, H., Torres-Martinez, S., Chen, W., Garre, V., 2016. A new regulatory mechanism controlling carotenogenesis in the fungus Mucor circinelloides as a target to generate β-carotene over-producing strains by genetic engineering. Microb. Cell Fact. 15. doi.org/10.1186/s12934-016-0493-8

The disclosure of each patent and scientific document, computer program and algorithm referred to in this specification is incorporated by reference in its entirety.

Throughout the specification, the aim has been to describe typical aspects and embodiments without limiting the invention as disclosed herein to any one aspect, embodiment, or specific collection of features. It will be appreciated that various changes and modifications may be made relative to the exemplary disclosure provided herein without departing from the present invention.

In this specification, the use of the terms “suitable” and “suitably”, and similar terms, is not to be read as implying that a feature or step is essential, although such features or steps referred to as “suitable” may well be preferred.

In this specification, the indefinite articles “a” and “an” are not to be read as singular indefinite articles or as otherwise excluding more than one or more than a single subject to which the indefinite article refers. For example, “a” cell includes one cell, one or more cells, and a plurality of cells.

In this specification, the terms “comprises”, “comprising”, “includes”, “including”, and similar terms, are intended to denote the inclusion of a stated integer or integers, but not necessarily the exclusion of another integer or other integers, depending on context. That is, a product, composition, or method, etc., that comprises or includes stated integer(s) need not have those integer(s) solely, and may well have at least some other integers not stated, depending on context.

In this specification, the terms “consisting essentially of” and “consists essentially of” are intended to mean a non-exclusive inclusion only to the extent that, if additional elements are included beyond those elements recited, the additional elements do not materially alter basic and novel characteristics. That is, a composition, apparatus, system, or method that “consists essentially of” one or more recited elements includes those elements only, or those elements and any additional elements that do not materially alter the basic and novel characteristics of the apparatus, system, or method.

### Tables

### Table Descriptions

Table 1. Kinetic model parameters as per Example 1.

Table 2. Nitrogen sources assessed in relation to growth and carotenoid production as per Example 1.

Table 3. Carbon sources assessed in relation to growth and carotenoid production as per Example 1.

Table 4. Genomic characteristics of X. dendrorhous strains (referred to as P. rhodozyma in Table 4) sequenced as per Example 1.

Tables 5-7. Analyses of variance of the factorial design as per Example 1 to evaluate the effect on carotenoid production of the culture media components: Bactopeptone (BP), Malt Extract (ME), Yeast Extract (YE), Sucrose, Dried Corn Steep Liquor (DCSL), Vitamins Cocktail, and Potassium Phosphate Monobasic (KH₂PO₄).

Table 8. Coefficients of the quadratic equation and model validation as per Example 1.

Table 9. Fermentation parameters of wild-type (CBS6938) and mutant (BPAX-A1) X. dendrorhous strains as per Example 2.

Table 10. Kinetic parameters of fermentation with wild-type (CBS6938) and mutant strains (BPAX-A1) of X. dendrorhous as per Example 2.

Table 11. Amino acid profile analysis for formulations as described in Example 3.

Table 12. Variants in re-sequenced X. dendrorhous CBS 6938 and eight mutant X. dendrorhous strains (MAMY3, MAMY6, MB18, MB24, MYM0, MYM6, MYM44, and MYM92) as per Example 1, relative to SEQ ID NOs:12685-12950. V=variation index; S=strain, wherein WT=re-sequenced CBS 6938, M1=MAMY3, M2=MAMY6, M3=MB18, M4=MB24, M5=MYM0, M6=MYM6, M7=MYM44, M8=MYM92); RS=reference scaffold in published genome assembly of Sharma et al. BMC genomics 16.1 (2015): 1-13, corresponding to SEQ ID NOs:12685-12950, wherein the scaffold number is given, i.e. ‘62’ corresponds to ‘scaffold_62’ as per the published assembly—the correspondence of SEQ ID NOs:12685-12950 to the reference scaffolds in the published genome assembly will be apparent or readily determinable by the skilled person; RB=reference scaffold base at the variation position; SB=strain base at the variation position; T=type of change of the variation, wherein 1=synonymous, 2=missense, 3=upstream region of a CDS, 4=downstream region of a CDS, 5=stop gained, 6=intergenic; 7=intron variant, 8=splice variant, 9=start lost; CDS=coding sequence reference for affected CDS (if applicable) as per the published genome assembly of Sharma et al. BMC genomic 16.1 (2015): 1-13, corresponding to SEQ ID NOs:12951-19331—the correspondence of SEQ ID NOs:12951-19331 to the CDS reference in the published genome assembly will be apparent or readily determinable by the skilled person.

Table 13. Variations (a subset of those in Table 12) shared across all eight sequenced mutant strains X. dendrorhous strains (MAMY3, MAMY6, MB18, MB24, MYM0, MYM6, MYM44, and MYM92) and not present in re-sequenced X. dendrorhous CBS 6938 as per Example 1, relative to SEQ ID NOs:12685-12950. V, RS, RP, RB, SB, T, and CDS are as per Table 12. GC=gene characterisation; AA change=amino acid change of the variation, as applicable.

Table 14. Twenty-five selected variations (Z₁ to Z₂₅; a subset of those in Table 13) considered of particular interest as per Example 1.

Table 15. Twenty-six selected variations (V₁ to V₂₆; a subset of those in Table 13) considered of particular interest as per Example 2.

Table 16. CDS transcripts differentially expressed between BPAX-A1 (MYM0) mutant X. dendrorhous strain and wild-type X. dendrorhous CBS 6938 as per Example 2.

Table 17. Differential expression analysis between BPAX-A1 (MYM0) mutant X. dendrorhous strain and wild-type X. dendrorhous CBS 6938 for selected CDS transcripts (Ti to T₅₅) considered of particular interest.

Table 18. CDS sequences encoding proteins differentially expressed between BPAX-A1 (MYM0) mutant X. dendrorhous strain and wild-type X. dendrorhous CBS 6938 at Growth Phase 3, as per Example 3.

Table 19. CDS sequences encoding proteins differentially expressed between BPAX-A1 (MYM0) mutant X. dendrorhous strain and wild-type X. dendrorhous CBS 6938 at Growth Phase 4, as per Example 3.

Table 20. CDS accessions of X. dendrorhous CBS 6938 (as published by Sharma et al. BMC genomics 16.1 (2015): 1-13 and deposited at the European Nucleotide Archive; www.ebi.ac.uk/ena/browser/home) containing variations as set out in Table 14 and Table 15, and corresponding SEQ ID NOs.

### Tables 1-20 Follow

TABLE 1

| Parameter | Unit | Value |
|---|---|---|
| μmax | h⁻¹ | 0.12 |
| Ks | g/L | 2.33 |
| Yxs | g/g | 0.7 |
| α | μg/gDCW | *2000-4000 |
| rho | — | 1 |
| kp | μg | 650,000 |
| β | μg/h | 50 |
| ms | g/gDCW · h | 0.005 |

*Low Range: Growth Phase; High Range: Production Phase

TABLE 2

| Nitrogen | | | |
|---|---|---|---|
| Source | A₆₀₀ | Carotenoids (μg/L) | Ypx (g/gDCW) |
| Urea | 18.6 ± 0.23 | 10511.63 ± 394.66 | 1339.23 ± 50.28 |
| Ammonium | 21.76 ± 0.12 | 26186.05 ± 2565.32 | 2848.84 ± 197.90 |
| Sulphate | | | |
| YE | 20.46 ± 0.31 | 41860.47 ± 1578.66 | 4850.21 ± 256.59 |
| ME | 9.08 ± 0.56 | 20186.05 ± 920.88 | 5285.83 ± 569.63 |
| BP | 20.42 ± 0.82 | 38046.51 ± 1578.66 | 4422.41 ± 360.84 |
| DCSL | 18.80 ± 0.46 | 29860.47 ± 2104.88 | 3768.083 ± 356.02 |

YE yeast extract; ME malt extract; BP bactopeptone; DCSL dried corn steep liquor; Ypx yield of carotenoid produced per gram of dried cell weight.

TABLE 3

| Carbon Source | A₆₀₀ | Final pH | Carotenoids (ug/L) | Ypx (g/g) |
|---|---|---|---|---|
| Molasses | 19.79 ± 0.12 | 5.39 ± 0.01 | 39906.98 ± 789.32 | 4778.89 ± 125.5 |
| Glucose | 26.00 ± 1.27 | 5.26 ± 0.01 | 37348.84 ± 2039.01 | 3132.13 ± 749.62 |
| Glycerol¹ | 27.89 ± 6.8 | 5.11 ± 0.01 | 53395.35 ± 1183.99 | 4688 ± 1244.11 |
| Sucrose | 26.55 ± 2.12 | 5.23 ± 0.06 | 36046.51 ± 2696.87 | 3217.91 ± 16.40 |

¹Lag phase extended for three days

TABLE 4

| | Genome | | | |
|---|---|---|---|---|
| | Size | No. of | | |
| Strain | (Mb) | contigs | % GC | N50 |
| P. rhodozyma CBS6938¹ | 19.50 | 266 | 47.31 | 2,080,000 |
| P. rhodozyma CBS6938_Y8² | 19.05 | 1239 | 48.31 | 39,444 |
| P. rhodozyma MYM6_Y2 | 19.01 | 1254 | 48.51 | 37,281 |
| P. rhodozyma MYM0_Y11 | 19.01 | 1136 | 48.61 | 42,199 |
| P. rhodozyma MYM44_Y14 | 18.92 | 1722 | 48.72 | 23,350 |
| P. rhodozyma MB18_Y15 | 19.01 | 1140 | 48.64 | 44,235 |
| P. rhodozyma MAMY3_Y16 | 18.88 | 1556 | 48.64 | 28,341 |
| P. rhodozyma MB24_Y17 | 19.11 | 1421 | 48.73 | 30,813 |
| P. rhodozyma MYM92_Y4 | 18.96 | 1651 | 48.76 | 23,938 |
| P. rhodozyma MAMY6_Y12 | 18.55 | 1565 | 48.27 | 23,723 |

¹Phaffia rhodozyma CBS 6938 downloaded from ENA: LN483084-LN483350 (Sharma et al., 2015).
²Phaffia rhodozyma CBS 6938 re- sequenced in this study.
% GC, guanine-cytosine percentage content.

TABLE 5

| Source | DF | Adj SS | Adj MS | F-Value | P-Value |
|---|---|---|---|---|---|
| Model | 7 | 1445027042 | 206432435 | 112.30 | 0.000 |
| Linear | 7 | 1445027042 | 206432435 | 112.30 | 0.000 |
| BP | 1 | 8450514 | 8450514 | 4.60 | 0.064 |
| ME | 1 | 109032450 | 109032450 | 59.31 | 0.000 |
| YE | 1 | 14018929 | 14018929 | 7.63 | 0.025 |
| Sucrose | 1 | 1284305571 | 1284305571 | 698.64 | 0.000 |
| DCSL | 1 | 22287182 | 22287182 | 12.12 | 0.008 |
| Vitamins Cocktail | 1 | 6905895 | 6905895 | 3.76 | 0.089 |
| KH2PO4 | 1 | 26501 | 26501 | 0.01 | 0.907 |
| Error | 8 | 14706328 | 1838291 | | |
| Total | 15 | 1459733369 | | | |

R² = 98.11%

TABLE 6

| Source | DF | Adj SS | Adj MS | F-Value | P-Value |
|---|---|---|---|---|---|
| Model | 7 | 38.3215 | 5.4745 | 49.73 | 0.000 |
| Linear | 7 | 38.3215 | 5.4745 | 49.73 | 0.000 |
| BP | 1 | 0.4390 | 0.4390 | 3.99 | 0.081 |
| ME | 1 | 1.7224 | 1.7224 | 15.65 | 0.004 |
| YE | 1 | 0.0579 | 0.0579 | 0.53 | 0.489 |
| Sucrose | 1 | 35.7573 | 35.7573 | 324.85 | 0.000 |
| DCSL | 1 | 0.1411 | 0.1411 | 1.28 | 0.290 |
| Vitamins Cocktail | 1 | 0.0752 | 0.0752 | 0.68 | 0.432 |
| KH2PO4 | 1 | 0.1287 | 0.1287 | 1.17 | 0.311 |
| Error | 8 | 0.8806 | 0.1101 | | |
| Total | 15 | 39.2021 | | | |

R² = 97.75%

TABLE 7

| Source | DF | Adj SS | Adj MS | F-Value | P-Value |
|---|---|---|---|---|---|
| Model | 7 | 2009531 | 287076 | 13.55 | 0.001 |
| Linear | 7 | 2009531 | 287076 | 13.55 | 0.001 |
| BP | 1 | 151052 | 151052 | 7.13 | 0.028 |
| ME | 1 | 174941 | 174941 | 8.26 | 0.021 |
| YE | 1 | 151802 | 151802 | 7.16 | 0.028 |
| Sucrose | 1 | 1187459 | 1187459 | 56.05 | 0.000 |
| DCSL | 1 | 303517 | 303517 | 14.33 | 0.005 |
| Vitamins Cocktail | 1 | 35727 | 35727 | 1.69 | 0.230 |
| KH2PO4 | 1 | 5032 | 5032 | 0.24 | 0.639 |
| Error | 8 | 169496 | 21187 | | |
| Total | 15 | 2179026 | | | |

R² = 92.22%

TABLE 8

| initial sucrose | Carotenoid | Carotenoid |
|---|---|---|
| concentration | production (ug/L) | production (ug/L) |
| (g/L) | Predicted | Calculated |
| 10 | 35,849 | 44,418 |
| 15 | 48,217 | 51,162 |
| 25 | 74,793 | 73,953 |
| 40 | 118,636 | 111,860 |
| 80 | 165,546 | 145,348 |

| Coded Coefficient | Value | P-Value |
|---|---|---|
| Constant | 91529 | 0.000 |
| ME | 4175 | 0.447 |
| Sucrose | 31769 | 0.000 |
| DCSL | −7620 | 0.191 |
| YE | 2882 | 0.597 |
| ME*ME | −4079 | 0.696 |
| Sucrose*Sucrose | −12521 | 0.241 |
| DCSL*DCSL | −5591 | 0.579 |
| YE*YE | −6335 | 0.545 |
| ME*Sucrose | −3805 | 0.783 |
| ME*DCSL | −5520 | 0.684 |
| ME*YE | 6234 | 0.653 |
| Sucrose*DCSL | −14251 | 0.302 |
| Sucrose*YE | 10433 | 0.454 |
| DCSL*YE | −524 | 0.969 |

TABLE 9

| | Biomass | Carotenoids | Astaxanthin | Ypx | Ypax | Yxs |
|---|---|---|---|---|---|---|
| Strain | (gDCW/L) | (μg/L) | (μg/L) | (μg/gDCW) | (μg/gDCW) | (gDCW/g) |
| BPAX-A1 | 10.01 ± 0.39 | 28310 ± 12 | 21212 ± 19 | 2832 ± 207 | 2118 ± 165 | 0.43 ± 0.02 |
| CBS6938 | 13.60 ± 0.29 | 2651 ± 61 | 1875 ± 29 | 195 ± 0.5 | 137 ± 1.7 | 0.62 ± 0.01 |

The parameters represent global values of the average of three biological replicates. Ypx Specific carotenoid production yield; Ypax Specific AX production yield; Yxs Biomass to sugar consumption yield. gDCW/L grams of dried cell weight per litre.

TABLE 10

| | μ | qp | qpax | qs |
|---|---|---|---|---|
| Strain | (h⁻¹) | (μg/gDCW · h) | (μg/gDCW · h) | (g/gDCW · h) |
| BPAX-A1-Phase 1 | 0.11 ± 0.002 | 80.70 ± 16.39 | 50.25 ± 4.46 | 0.13 ± 0.33 |
| BPAX-A1-Phase 2 | 0.10 ± 0.005 | 197.37 ± 15.62 | 67.01 ± 17.93 | 0.22 ± 0.01 |
| BPAX-A1-Phase 3 | 0.10 ± 0.004 | 141.89 ± 27.55 | 60.58 ± 2.19 | 0.21 ± 0.03 |
| BPAX-A1-Phase 4 | 0.0 ± 0.000 | 83.60 ± 1.15 | 33.37 ± 7.79 | 0.0 ± 0.00 |
| CBS6938-Phase 1 | 0.12 ± 0.002 | 2.14 ± 0.05 | 1.27 ± 0.50 | 0.18 ± 0.03 |
| CBS6938-Phase 2 | 0.12 ± 0.004 | 16.97 ± 0.20 | 3.02 ± 1.41 | 0.25 ± 0.02 |
| CBS6938-Phase 3 | 0.10 ± 0.004 | 16.36 ± 0.27 | 9.55 ± 2.43 | 0.19 ± 0.01 |
| CBS6938-Phase 4 | 0.0 ± 0.000 | 3.90 ± 0.13 | 4.03 ± 2.55 | 0.0 ± 0.00 |

μ: Specific growth rate; qp: Specific carotenoids production; qax: Specific astaxanthin production; qs: Specific glucose consumption

TABLE 11

| Amino Acid | uM/gDCW | SSD |
|---|---|---|
| Arginine | 22.23377 | 1.296117 |
| Histidine | 0.679908 | 0.115273 |
| Threonine | 3.79767 | 0.511533 |
| Glutamine/Lysine | 34.24658 | 3.448584 |
| Lysine/Glutamine | 1.051276 | 0.149964 |
| Valine | 2.577432 | 0.475985 |
| Methionine | 0.266643 | 0.025636 |
| Isoleucine | 0.460132 | 0.080446 |
| Leucine | 0.460132 | 0.080446 |
| Phenylalanine | 0.612914 | 0.118322 |
| Tryptophan | 0.049219 | 0.006615 |
| L-Tyrosine | 0.141318 | 0.019971 |
| Proline | 0.262457 | 0.039893 |
| Serine | 4.693926 | 0.556659 |
| Aspartic acid | 21.66696 | 1.089912 |
| Glutamic acid | 57.78015 | 6.399202 |
| Glycine | 0.699527 | 0.138553 |
| Asparagine | 7.194973 | 0.968078 |
| Alanine | 5.183911 | 1.019159 |

TABLE 12

| V | S | RS | RP | RB | SB | T | CDS |
|---|---|---|---|---|---|---|---|
| X₁ | WT | 62 | 26 | T | A | 4 | CDZ96152 |
| X₂ | WT | 62 | 27 | T | C | 4 | CDZ96152 |
| X₃ | WT | 62 | 55 | T | A | 4 | CDZ96152 |
| X₄ | WT | 179 | 2648 | A | G | 4 | CDZ96154 |
| X₅ | WT | 179 | 2649 | G | A | 4 | CDZ96154 |
| X₆ | WT | 24 | 513 | T | C | 3 | CDZ96160 |
| X₇ | WT | 24 | 520 | C | G | 3 | CDZ96160 |
| X₈ | WT | 24 | 242334 | A | T | 5 | CDZ96235 |
| X₉ | WT | 33 | 399 | T | A | 4 | CED82002 |
| X₁₀ | WT | 33 | 4019 | T | C | 4 | CED82002 |
| X₁₁ | WT | 33 | 370630 | A | T | 6 | CED82121- |
| | | | | | | | CED82122 |
| X₁₂ | WT | 33 | 374131 | A | G | 6 | CED82121- |
| | | | | | | | CED82122 |
| X₁₃ | WT | 33 | 374134 | A | G | 6 | CED82121- |
| | | | | | | | CED82122 |
| X₁₄ | WT | 33 | 374137 | A | T | 6 | CED82121- |
| | | | | | | | CED82122 |
| X₁₅ | WT | 33 | 374150 | A | C | 6 | CED82121- |
| | | | | | | | CED82122 |
| X₁₆ | WT | 33 | 375339 | A | G | 6 | CED82121- |
| | | | | | | | CED82122 |
| X₁₇ | WT | 33 | 375366 | T | C | 6 | CED82121- |
| | | | | | | | CED82122 |
| X₁₈ | WT | 33 | 385319 | A | C | 2 | CED82122 |
| X₁₉ | WT | 33 | 385527 | C | T | 2 | CED82122 |
| X₂₀ | WT | 33 | 917545 | A | G | 7 | CED82294 |
| X₂₁ | WT | 33 | 917558 | G | A | 7 | CED82294 |
| X₂₂ | WT | 33 | 917771 | A | T | 7 | CED82294 |
| X₂₃ | WT | 33 | 928726 | C | T | 3 | CED82295 |
| X₂₄ | WT | 33 | 936350 | T | A | 3 | CED82300 |
| X₂₅ | WT | 33 | 936356 | G | A | 3 | CED82300 |
| X₂₆ | WT | 33 | 936571 | A | G | 3 | CED82300 |
| X₂₇ | WT | 33 | 936600 | G | A | 3 | CED82300 |
| X₂₈ | WT | 33 | 936667 | A | G | 3 | CED82300 |
| X₂₉ | WT | 33 | 1538001 | G | A | 3 | CED82483 |
| X₃₀ | WT | 33 | 1544767 | A | C | 6 | CED82485- |
| | | | | | | | CED82486 |
| X₃₁ | WT | 48 | 563 | C | T | 6 | |
| X₃₂ | WT | 48 | 565 | T | C | 6 | |
| X₃₃ | WT | 48 | 576 | G | A | 6 | |
| X₃₄ | WT | 48 | 580 | T | A | 6 | |
| X₃₅ | WT | 48 | 720 | A | G | 6 | |
| X₃₆ | WT | 52 | 516267 | C | T | 3 | CED82823 |
| X₃₇ | WT | 52 | 1313179 | A | G | 3 | CED83105 |
| X₃₈ | WT | 52 | 1506995 | T | C | 2 | CED83177 |
| X₃₉ | WT | 52 | 1874150 | G | T | 3 | CED83305 |
| X₄₀ | WT | 52 | 2575090 | A | G | 3 | CED83525 |
| X₄₁ | WT | 54 | 4815 | G | A | 1 | CDZ96384 |
| X₄₂ | WT | 54 | 4816 | A | T | 2 | CDZ96384 |
| X₄₃ | WT | 54 | 4817 | G | C | 2 | CDZ96384 |
| X₄₄ | WT | 54 | 4821 | G | A | 1 | CDZ96384 |
| X₄₅ | WT | 54 | 274617 | A | T | 3 | CDZ96471 |
| X₄₆ | WT | 54 | 316790 | T | G | 4 | CDZ96487 |
| X₄₇ | WT | 54 | 468399 | T | A | 3 | CDZ96530 |
| X₄₈ | WT | 54 | 616268 | A | G | 3 | CDZ96581 |
| X₄₉ | WT | 58 | 93 | T | C | 6 | |
| X₅₀ | WT | 69 | 82710 | G | A | 3 | CED83554 |
| X₅₁ | WT | 69 | 1084824 | G | A | 3 | CED83891 |
| X₅₂ | WT | 69 | 1084835 | C | T | 3 | CED83891 |
| X₅₃ | WT | 69 | 1084848 | A | G | 3 | CED83891 |
| X₅₄ | WT | 69 | 1084866 | A | G | 3 | CED83891 |
| X₅₅ | WT | 69 | 1084931 | C | T | 3 | CED83891 |
| X₅₆ | WT | 69 | 1109785 | C | T | 3 | CED83899 |
| X₅₇ | WT | 69 | 1109813 | A | G | 3 | CED83899 |
| X₅₈ | WT | 69 | 1109818 | A | G | 3 | CED83899 |
| X₅₉ | WT | 69 | 1109819 | T | A | 3 | CED83899 |
| X₆₀ | WT | 69 | 1109823 | T | C | 3 | CED83899 |
| X₆₁ | WT | 69 | 1109830 | A | G | 3 | CED83899 |
| X₆₂ | WT | 69 | 1109831 | C | A | 3 | CED83899 |
| X₆₃ | WT | 69 | 1109835 | A | G | 3 | CED83899 |
| X₆₄ | WT | 69 | 1257466 | A | G | 2 | CED83949 |
| X₆₅ | WT | 69 | 2089848 | A | G | 4 | CED84232 |
| X₆₆ | WT | 69 | 2089870 | C | T | 4 | CED84232 |
| X₆₇ | WT | 69 | 2089876 | C | A | 4 | CED84232 |
| X₆₈ | WT | 69 | 2099452 | C | G | 4 | CED84233 |
| X₆₉ | WT | 72 | 93 | T | C | 6 | |
| X₇₀ | WT | 77 | 18912 | A | G | 2 | CDZ96635 |
| X₇₁ | WT | 77 | 34978 | G | T | 3 | CDZ96642 |
| X₇₂ | WT | 77 | 39665 | G | C | 2 | CDZ96646 |
| X₇₃ | WT | 77 | 39842 | G | A | 8 | CDZ96646 |
| X₇₄ | WT | 78 | 263674 | A | G | 3 | CED84438 |
| X₇₅ | WT | 78 | 263828 | T | C | 3 | CED84438 |
| X₇₆ | WT | 78 | 263896 | T | C | 3 | CED84438 |
| X₇₇ | WT | 78 | 280587 | G | A | 2 | CED84440 |
| X₇₈ | WT | 78 | 280603 | A | G | 1 | CED84440 |
| X₇₉ | WT | 78 | 281454 | C | A | 3 | CED84441 |
| X₈₀ | WT | 78 | 298141 | C | G | 3 | CED84443 |
| X₈₁ | WT | 78 | 301815 | T | C | 3 | CED84443 |
| X₈₂ | WT | 78 | 302700 | A | G | 2 | CED84443 |
| X₈₃ | WT | 78 | 333312 | A | G | 3 | CED84452 |
| X₈₄ | WT | 78 | 661166 | G | C | 2 | CED84562 |
| X₈₅ | WT | 78 | 1143063 | A | G | 3 | CED84728 |
| X₈₆ | WT | 78 | 1742796 | G | A | 3 | CED84926 |
| X₈₇ | WT | 79 | 161 | T | C | 3 | CDZ96674 |
| X₈₈ | WT | 79 | 687842 | T | C | 2 | CDZ96908 |
| X₈₉ | WT | 79 | 735053 | T | C | 3 | CDZ96928 |
| X₉₀ | WT | 79 | 748083 | G | T | 3 | CDZ96929 |
| X₉₁ | WT | 79 | 759403 | C | T | 3 | CDZ96931 |
| X₉₂ | WT | 79 | 759659 | G | A | 2 | CDZ96931 |
| X₉₃ | WT | 79 | 760430 | T | C | 2 | CDZ96932 |
| X₉₄ | WT | 79 | 760541 | A | G | 2 | CDZ96932 |
| X₉₅ | WT | 79 | 760891 | A | G | 2 | CDZ96932 |
| X₉₆ | WT | 79 | 760988 | G | T | 2 | CDZ96932 |
| X₉₇ | WT | 79 | 761056 | T | C | 3 | CDZ96932 |
| X₉₈ | WT | 79 | 768537 | A | G | 4 | CDZ96933 |
| X₉₉ | WT | 79 | 786064 | A | G | 3 | CDZ96934 |
| X₁₀₀ | WT | 79 | 830852 | T | A | 2 | CDZ96950 |
| X₁₀₁ | WT | 79 | 1287496 | C | T | 1 | CDZ97107 |
| X₁₀₂ | WT | 79 | 2339881 | C | G | 3 | CDZ97460 |
| X₁₀₃ | WT | 79 | 2340326 | G | A | 3 | CDZ97462 |
| X₁₀₄ | WT | 79 | 2340636 | T | C | 3 | CDZ97462 |
| X₁₀₅ | WT | 81 | 210 | G | T | 6 | |
| X₁₀₆ | WT | 85 | 521 | C | T | 6 | |
| X₁₀₇ | WT | 85 | 522 | A | G | 6 | |
| X₁₀₈ | WT | 105 | 130 | T | A | 2 | CDZ97474 |
| X₁₀₉ | WT | 105 | 429 | C | T | 4 | CDZ97474 |
| X₁₁₀ | WT | 121 | 784 | A | G | 6 | |
| X₁₁₁ | WT | 121 | 1269 | A | T | 6 | |
| X₁₁₂ | WT | 121 | 1402 | C | G | 6 | |
| X₁₁₃ | WT | 138 | 123 | G | T | 6 | |
| X₁₁₄ | WT | 138 | 651 | T | A | 6 | |
| X₁₁₅ | WT | 138 | 657 | A | C | 6 | |
| X₁₁₆ | WT | 149 | 1163 | T | C | 8 | CDZ97597 |
| X₁₁₇ | WT | 149 | 2074 | T | G | 3 | CDZ97598 |
| X₁₁₈ | WT | 159 | 106 | A | C | 6 | |
| X₁₁₉ | WT | 162 | 68192 | C | G | 2 | CDZ97625 |
| X₁₂₀ | WT | 162 | 70405 | A | G | 1 | CDZ97625 |
| X₁₂₁ | WT | 162 | 308369 | T | C | 1 | CDZ97702 |
| X₁₂₂ | WT | 162 | 311666 | A | G | 4 | CDZ97702 |
| X₁₂₃ | WT | 162 | 320785 | G | C | 4 | CDZ97703 |
| X₁₂₄ | WT | 162 | 320786 | T | G | 4 | CDZ97703 |
| X₁₂₅ | WT | 162 | 321460 | C | G | 4 | CDZ97703 |
| X₁₂₆ | WT | 162 | 321463 | A | C | 4 | CDZ97703 |
| X₁₂₇ | WT | 162 | 322591 | G | A | 4 | CDZ97703 |
| X₁₂₈ | WT | 162 | 323448 | T | G | 2 | CDZ97703 |
| X₁₂₉ | WT | 162 | 323451 | T | G | 2 | CDZ97703 |
| X₁₃₀ | WT | 162 | 323735 | A | G | 1 | CDZ97703 |
| X₁₃₁ | WT | 162 | 324114 | T | C | 2 | CDZ97703 |
| X₁₃₂ | WT | 162 | 324139 | C | T | 2 | CDZ97703 |
| X₁₃₃ | WT | 162 | 324568 | A | G | 2 | CDZ97703 |
| X₁₃₄ | WT | 162 | 492101 | G | A | 3 | CDZ97764 |
| X₁₃₅ | WT | 162 | 622253 | A | C | 1 | CDZ97809 |
| X₁₃₆ | WT | 162 | 738675 | G | A | 3 | CDZ97842 |
| X₁₃₇ | WT | 162 | 738686 | C | T | 3 | CDZ97842 |
| X₁₃₈ | WT | 189 | 69196 | T | A | 3 | CDZ97980 |
| X₁₃₉ | WT | 189 | 194647 | A | G | 3 | CDZ98020 |
| X₁₄₀ | WT | 189 | 194665 | A | G | 3 | CDZ98020 |
| X₁₄₁ | WT | 191 | 342 | A | G | 3 | CDZ98107 |
| X₁₄₂ | WT | 191 | 347 | C | T | 3 | CDZ98107 |
| X₁₄₃ | WT | 191 | 427 | C | T | 3 | CDZ98107 |
| X₁₄₄ | WT | 191 | 451 | C | T | 3 | CDZ98107 |
| X₁₄₅ | WT | 191 | 487 | A | G | 3 | CDZ98107 |
| X₁₄₆ | WT | 198 | 202 | C | A | 6 | |
| X₁₄₇ | WT | 198 | 203 | G | A | 6 | |
| X₁₄₈ | WT | 206 | 406 | G | A | 6 | |
| X₁₄₉ | WT | 220 | 116 | C | G | 6 | |
| X₁₅₀ | WT | 223 | 93 | G | A | 4 | CDZ98118 |
| X₁₅₁ | WT | 223 | 102 | A | G | 4 | CDZ98118 |
| X₁₅₂ | WT | 223 | 133 | A | G | 4 | CDZ98118 |
| X₁₅₃ | WT | 223 | 150 | T | C | 4 | CDZ98118 |
| X₁₅₄ | WT | 223 | 155 | A | C | 4 | CDZ98118 |
| X₁₅₅ | WT | 223 | 158 | T | C | 4 | CDZ98118 |
| X₁₅₆ | WT | 223 | 160 | C | T | 4 | CDZ98118 |
| X₁₅₇ | WT | 223 | 175 | G | A | 4 | CDZ98118 |
| X₁₅₈ | WT | 223 | 177 | A | G | 4 | CDZ98118 |
| X₁₅₉ | WT | 223 | 188 | A | G | 4 | CDZ98118 |
| X₁₆₀ | WT | 223 | 192 | C | A | 4 | CDZ98118 |
| X₁₆₁ | WT | 223 | 194 | T | C | 4 | CDZ98118 |
| X₁₆₂ | WT | 223 | 195 | T | G | 4 | CDZ98118 |
| X₁₆₃ | WT | 223 | 197 | T | C | 4 | CDZ98118 |
| X₁₆₄ | WT | 229 | 225 | G | C | 6 | |
| X₁₆₅ | WT | 235 | 3161 | A | G | 6 | |
| X₁₆₆ | WT | 249 | 206 | G | A | 4 | CED84929 |
| X₁₆₇ | WT | 249 | 29505 | T | G | 3 | CED84937 |
| X₁₆₈ | WT | 249 | 41573 | G | A | 3 | CED84940 |
| X₁₆₉ | WT | 249 | 107121 | A | G | 3 | CED84966 |
| X₁₇₀ | WT | 249 | 501510 | T | C | 3 | CED85093 |
| X₁₇₁ | WT | 249 | 969333 | G | T | 3 | CED85238 |
| X₁₇₂ | WT | 249 | 1195367 | G | A | 3 | CED85307 |
| X₁₇₃ | WT | 249 | 1498029 | T | C | 3 | CED85415 |
| X₁₇₄ | WT | 249 | 1498823 | T | C | 2 | CED85414 |
| X₁₇₅ | WT | 249 | 1498829 | C | T | 2 | CED85414 |
| X₁₇₆ | WT | 249 | 1499008 | A | G | 3 | CED85415 |
| X₁₇₇ | WT | 249 | 1502631 | G | T | 2 | CED85416 |
| X₁₇₈ | WT | 249 | 1502641 | T | A | 2 | CED85416 |
| X₁₇₉ | WT | 249 | 1502819 | T | G | 1 | CED85416 |
| X₁₈₀ | WT | 249 | 1505138 | G | A | 3 | CED85416 |
| X₁₈₁ | WT | 249 | 1505634 | T | G | 3 | CED85416 |
| X₁₈₂ | WT | 249 | 1518591 | T | A | 3 | CED85419 |
| X₁₈₃ | WT | 249 | 1519431 | A | G | 3 | CED85419 |
| X₁₈₄ | WT | 249 | 1653615 | T | G | 2 | CED85457 |
| X₁₈₅ | WT | 249 | 1653962 | C | T | 4 | CED85458 |
| X₁₈₆ | WT | 249 | 1679374 | A | G | 3 | CED85461 |
| X₁₈₇ | WT | 249 | 1679375 | A | T | 3 | CED85461 |
| X₁₈₈ | WT | 249 | 1780551 | A | G | 3 | CED85493 |
| X₁₈₉ | WT | 249 | 1780569 | A | G | 3 | CED85493 |
| X₁₉₀ | WT | 249 | 1801965 | G | A | 3 | CED85501 |
| X₁₉₁ | WT | 249 | 2255547 | G | C | 3 | CED85640 |
| X₁₉₂ | WT | 258 | 166 | A | G | 6 | |
| X₁₉₃ | WT | 258 | 576 | A | G | 6 | |
| X₁₉₄ | WT | 258 | 1139 | A | T | 6 | |
| X₁₉₅ | WT | 262 | 4049 | G | A | 1 | CDZ98330 |
| X₁₉₆ | WT | 262 | 136093 | A | T | 1 | CDZ98374 |
| X₁₉₇ | WT | 262 | 539428 | G | A | 3 | CDZ98514 |
| X₁₉₈ | WT | 262 | 539452 | A | G | 3 | CDZ98514 |
| X₁₉₉ | WT | 262 | 539470 | A | G | 3 | CDZ98514 |
| X₂₀₀ | WT | 262 | 628911 | G | C | 3 | CDZ98543 |
| X₂₀₁ | WT | 262 | 638804 | G | C | 3 | CDZ98544 |
| X₂₀₂ | WT | 262 | 638986 | C | G | 3 | CDZ98544 |
| X₂₀₃ | WT | 262 | 641218 | G | C | 4 | CDZ98544 |
| X₂₀₄ | WT | 262 | 653920 | A | G | 4 | CDZ98545 |
| X₂₀₅ | WT | 262 | 655754 | A | T | 4 | CDZ98545 |
| X₂₀₆ | WT | 262 | 655757 | T | C | 4 | CDZ98545 |
| X₂₀₇ | WT | 262 | 941629 | C | T | 1 | CDZ98638 |
| X₂₀₈ | WT | 262 | 1329643 | C | T | 3 | CDZ98760 |
| X₂₀₉ | WT | 262 | 1678838 | C | A | 2 | CDZ98874 |
| X₂₁₀ | WT | 262 | 1716385 | G | A | 3 | CDZ98887 |
| X₂₁₁ | WT | 267 | 93 | G | A | 6 | |
| X₂₁₂ | WT | 267 | 94 | A | G | 6 | |
| X₂₁₃ | WT | 267 | 133 | T | C | 6 | |
| X₂₁₄ | WT | 267 | 134 | C | T | 6 | |
| X₂₁₅ | WT | 267 | 201 | T | C | 6 | |
| X₂₁₆ | WT | 267 | 208 | T | C | 6 | |
| X₂₁₇ | WT | 267 | 233 | A | G | 6 | |
| X₂₁₈ | WT | 267 | 234 | A | C | 6 | |
| X₂₁₉ | WT | 267 | 237 | A | G | 6 | |
| X₂₂₀ | WT | 267 | 254 | G | T | 6 | |
| X₂₂₁ | WT | 267 | 256 | A | G | 6 | |
| X₂₂₂ | WT | 267 | 257 | T | C | 6 | |
| X₂₂₃ | M1 | 22 | 1643 | C | T | 2 | CED80056 |
| X₂₂₄ | M1 | 22 | 2524 | G | A | 4 | CED80056 |
| X₂₂₅ | M1 | 28 | 236 | G | A | 3 | CDZ96150 |
| X₂₂₆ | M1 | 28 | 377 | G | A | 3 | CDZ96150 |
| X₂₂₇ | M1 | 28 | 1506 | C | T | 2 | CDZ96150 |
| X₂₂₈ | M1 | 28 | 1677 | C | T | 4 | CDZ96150 |
| X₂₂₉ | M1 | 43 | 464 | G | A | 2 | CDZ96151 |
| X₂₃₀ | M1 | 43 | 753 | C | T | 2 | CDZ96151 |
| X₂₃₁ | M1 | 43 | 757 | G | A | 1 | CDZ96151 |
| X₂₃₂ | M1 | 43 | 1527 | C | T | 3 | CDZ96151 |
| X₂₃₃ | M1 | 43 | 1748 | G | A | 3 | CDZ96151 |
| X₂₃₄ | M1 | 43 | 1928 | C | T | 3 | CDZ96151 |
| X₂₃₅ | M1 | 62 | 26 | T | A | 4 | CDZ96152 |
| X₂₃₆ | M1 | 62 | 27 | T | C | 4 | CDZ96152 |
| X₂₃₇ | M1 | 62 | 55 | T | A | 4 | CDZ96152 |
| X₂₃₈ | M1 | 62 | 403 | C | T | 1 | CDZ96152 |
| X₂₃₉ | M1 | 62 | 875 | G | A | 2 | CDZ96152 |
| X₂₄₀ | M1 | 102 | 339 | C | T | 4 | CDZ96153 |
| X₂₄₁ | M1 | 102 | 951 | G | A | 4 | CDZ96153 |
| X₂₄₂ | M1 | 102 | 1042 | G | A | 4 | CDZ96153 |
| X₂₄₃ | M1 | 102 | 2334 | G | A | 2 | CDZ96153 |
| X₂₄₄ | M1 | 102 | 2590 | C | T | 2 | CDZ96153 |
| X₂₄₅ | M1 | 102 | 3427 | C | T | 3 | CDZ96153 |
| X₂₄₆ | M1 | 176 | 358 | G | A | 3 | CED80058 |
| X₂₄₇ | M1 | 176 | 1872 | G | A | 4 | CED80058 |
| X₂₄₈ | M1 | 176 | 1980 | G | A | 4 | CED80058 |
| X₂₄₉ | M1 | 179 | 93 | A | G | 3 | CDZ96154 |
| X₂₅₀ | M1 | 179 | 96 | G | A | 3 | CDZ96154 |
| X₂₅₁ | M1 | 179 | 1525 | G | A | 7 | CDZ96154 |
| X₂₅₂ | M1 | 179 | 1780 | C | T | 7 | CDZ96154 |
| X₂₅₃ | M1 | 188 | 815 | C | T | 2 | CED80060 |
| X₂₅₄ | M1 | 188 | 1174 | C | T | 4 | CED80060 |
| X₂₅₅ | M1 | 188 | 2163 | G | A | 4 | CED80060 |
| X₂₅₆ | M1 | 188 | 2170 | G | A | 4 | CED80060 |
| X₂₅₇ | M1 | 247 | 102 | G | A | 4 | CED80061 |
| X₂₅₈ | M1 | 247 | 989 | C | T | 2 | CED80061 |
| X₂₅₉ | M1 | 247 | 1053 | G | A | 2 | CED80061 |
| X₂₆₀ | M1 | 7 | 160 | T | C | 6 | |
| X₂₆₁ | M1 | 8 | 1110 | C | T | 6 | |
| X₂₆₂ | M1 | 11 | 154 | A | C | 6 | |
| X₂₆₃ | M1 | 11 | 158 | C | T | 6 | |
| X₂₆₄ | M1 | 13 | 131 | G | C | 3 | CDZ96158 |
| X₂₆₅ | M1 | 15 | 134 | G | A | 6 | |
| X₂₆₆ | M1 | 15 | 135 | T | C | 6 | |
| X₂₆₇ | M1 | 15 | 139 | G | C | 6 | |
| X₂₆₈ | M1 | 15 | 148 | G | C | 6 | |
| X₂₆₉ | M1 | 15 | 149 | A | G | 6 | |
| X₂₇₀ | M1 | 15 | 165 | G | T | 6 | |
| X₂₇₁ | M1 | 15 | 184 | C | T | 6 | |
| X₂₇₂ | M1 | 15 | 205 | C | G | 6 | |
| X₂₇₃ | M1 | 15 | 214 | G | C | 6 | |
| X₂₇₄ | M1 | 15 | 248 | G | A | 6 | |
| X₂₇₅ | M1 | 15 | 249 | C | G | 6 | |
| X₂₇₆ | M1 | 15 | 252 | C | T | 6 | |
| X₂₇₇ | M1 | 15 | 254 | A | G | 6 | |
| X₂₇₈ | M1 | 15 | 256 | T | C | 6 | |
| X₂₇₉ | M1 | 15 | 327 | T | G | 6 | |
| X₂₈₀ | M1 | 24 | 513 | T | C | 3 | CDZ96160 |
| X₂₈₁ | M1 | 24 | 520 | C | G | 3 | CDZ96160 |
| X₂₈₂ | M1 | 24 | 5850 | C | T | 2 | CDZ96160 |
| X₂₈₃ | M1 | 24 | 68660 | C | T | 3 | CDZ96181 |
| X₂₈₄ | M1 | 24 | 118860 | C | T | 2 | CDZ96200 |
| X₂₈₅ | M1 | 24 | 168850 | C | T | 1 | CDZ96214 |
| X₂₈₆ | M1 | 24 | 180340 | G | A | 3 | CDZ96216 |
| X₂₈₇ | M1 | 24 | 197593 | C | T | 2 | CDZ96224 |
| X₂₈₈ | M1 | 24 | 266697 | G | A | 1 | CDZ96242 |
| X₂₈₉ | M1 | 24 | 278319 | G | A | 3 | CDZ96245 |
| X₂₉₀ | M1 | 24 | 289183 | T | C | 3 | CDZ96250 |
| X₂₉₁ | M1 | 24 | 307114 | G | A | 2 | CDZ96255 |
| X₂₉₂ | M1 | 24 | 340663 | T | C | 3 | CDZ96265 |
| X₂₉₃ | M1 | 24 | 340866 | G | A | 3 | CDZ96265 |
| X₂₉₄ | M1 | 24 | 363770 | C | T | 2 | CDZ96275 |
| X₂₉₅ | M1 | 24 | 382911 | C | T | 2 | CDZ96282 |
| X₂₉₆ | M1 | 24 | 400418 | C | T | 2 | CDZ96287 |
| X₂₉₇ | M1 | 24 | 420108 | G | A | 3 | CDZ96291 |
| X₂₉₈ | M1 | 24 | 474698 | G | A | 2 | CDZ96308 |
| X₂₉₉ | M1 | 26 | 245 | G | A | 6 | |
| X₃₀₀ | M1 | 27 | 445 | C | T | 6 | |
| X₃₀₁ | M1 | 27 | 1012 | A | C | 6 | |
| X₃₀₂ | M1 | 27 | 1013 | T | A | 6 | |
| X₃₀₃ | M1 | 32 | 158 | C | T | 3 | CDZ96332 |
| X₃₀₄ | M1 | 32 | 215 | C | T | 3 | CDZ96332 |
| X₃₀₅ | M1 | 32 | 2438 | C | T | 3 | CDZ96332 |
| X₃₀₆ | M1 | 32 | 2734 | C | T | 1 | CDZ96332 |
| X₃₀₇ | M1 | 32 | 2821 | C | T | 7 | CDZ96332 |
| X₃₀₈ | M1 | 32 | 3067 | C | T | 2 | CDZ96332 |
| X₃₀₉ | M1 | 32 | 3506 | C | T | 4 | CDZ96332 |
| X₃₁₀ | M1 | 32 | 3562 | G | A | 4 | CDZ96332 |
| X₃₁₁ | M1 | 33 | 399 | T | A | 4 | CED82002 |
| X₃₁₂ | M1 | 33 | 24784 | C | T | 2 | CED82008 |
| X₃₁₃ | M1 | 33 | 45488 | C | T | 3 | CED82012 |
| X₃₁₄ | M1 | 33 | 64676 | G | A | 3 | CED82019 |
| X₃₁₅ | M1 | 33 | 79913 | G | A | 1 | CED82024 |
| X₃₁₆ | M1 | 33 | 105276 | G | A | 2 | CED82036 |
| X₃₁₇ | M1 | 33 | 109397 | G | A | 2 | CED82038 |
| X₃₁₈ | M1 | 33 | 219700 | C | T | 2 | CED82074 |
| X₃₁₉ | M1 | 33 | 236149 | C | T | 2 | CED82078 |
| X₃₂₀ | M1 | 33 | 257786 | C | T | 1 | CED82084 |
| X₃₂₁ | M1 | 33 | 260027 | G | A | 3 | CED82084 |
| X₃₂₂ | M1 | 33 | 274627 | G | A | 8 | CED82091 |
| X₃₂₃ | M1 | 33 | 276128 | C | T | 2 | CED82092 |
| X₃₂₄ | M1 | 33 | 283808 | C | T | 2 | CED82093 |
| X₃₂₅ | M1 | 33 | 304408 | C | T | 4 | CED82099 |
| X₃₂₆ | M1 | 33 | 311532 | G | A | 3 | CED82101 |
| X₃₂₇ | M1 | 33 | 312032 | G | A | 3 | CED82101 |
| X₃₂₈ | M1 | 33 | 317713 | G | A | 1 | CED82104 |
| X₃₂₉ | M1 | 33 | 318242 | G | A | 3 | CED82105 |
| X₃₃₀ | M1 | 33 | 325745 | C | T | 2 | CED82108 |
| X₃₃₁ | M1 | 33 | 327840 | C | T | 1 | CED82108 |
| X₃₃₂ | M1 | 33 | 341890 | G | A | 2 | CED82113 |
| X₃₃₃ | M1 | 33 | 348487 | C | T | 2 | CED82115 |
| X₃₃₄ | M1 | 33 | 371277 | G | A | 6 | CED82121- |
| | | | | | | | CED82122 |
| X₃₃₅ | M1 | 33 | 374131 | A | G | 6 | CED82121- |
| | | | | | | | CED82122 |
| X₃₃₆ | M1 | 33 | 374134 | A | G | 6 | CED82121- |
| | | | | | | | CED82122 |
| X₃₃₇ | M1 | 33 | 374137 | A | T | 6 | CED82121- |
| | | | | | | | CED82122 |
| X₃₃₈ | M1 | 33 | 374150 | A | C | 6 | CED82121- |
| | | | | | | | CED82122 |
| X₃₃₉ | M1 | 33 | 375339 | A | G | 6 | CED82121- |
| | | | | | | | CED82122 |
| X₃₄₀ | M1 | 33 | 375366 | T | C | 6 | CED82121- |
| | | | | | | | CED82122 |
| X₃₄₁ | M1 | 33 | 382577 | C | T | 3 | CED82122 |
| X₃₄₂ | M1 | 33 | 383212 | G | A | 3 | CED82122 |
| X₃₄₃ | M1 | 33 | 385139 | G | C | 2 | CED82122 |
| X₃₄₄ | M1 | 33 | 385155 | T | C | 2 | CED82122 |
| X₃₄₅ | M1 | 33 | 385319 | A | C | 2 | CED82122 |
| X₃₄₆ | M1 | 33 | 448815 | G | A | 2 | CED82144 |
| X₃₄₇ | M1 | 33 | 453975 | C | T | 2 | CED82147 |
| X₃₄₈ | M1 | 33 | 455451 | C | T | 3 | CED82146 |
| X₃₄₉ | M1 | 33 | 509127 | C | T | 3 | CED82166 |
| X₃₅₀ | M1 | 33 | 626521 | C | T | 3 | CED82203 |
| X₃₅₁ | M1 | 33 | 639242 | G | A | 2 | CED82208 |
| X₃₅₂ | M1 | 33 | 699423 | C | T | 1 | CED82227 |
| X₃₅₃ | M1 | 33 | 774105 | G | A | 2 | CED82248 |
| X₃₅₄ | M1 | 33 | 792965 | G | A | 1 | CED82256 |
| X₃₅₅ | M1 | 33 | 816048 | C | T | 3 | CED82262 |
| X₃₅₆ | M1 | 33 | 822184 | G | A | 3 | CED82264 |
| X₃₅₇ | M1 | 33 | 917771 | A | T | 7 | CED82294 |
| X₃₅₈ | M1 | 33 | 933316 | C | T | 3 | CED82299 |
| X₃₅₉ | M1 | 33 | 936350 | T | A | 3 | CED82300 |
| X₃₆₀ | M1 | 33 | 936356 | G | A | 3 | CED82300 |
| X₃₆₁ | M1 | 33 | 936571 | A | G | 3 | CED82300 |
| X₃₆₂ | M1 | 33 | 936600 | G | A | 3 | CED82300 |
| X₃₆₃ | M1 | 33 | 936667 | A | G | 3 | CED82300 |
| X₃₆₄ | M1 | 33 | 938992 | C | T | 3 | CED82300 |
| X₃₆₅ | M1 | 33 | 1043825 | C | T | 2 | CED82333 |
| X₃₆₆ | M1 | 33 | 1049699 | G | A | 3 | CED82334 |
| X₃₆₇ | M1 | 33 | 1057631 | G | T | 8 | CED82339 |
| X₃₆₈ | M1 | 33 | 1082006 | G | A | 1 | CED82348 |
| X₃₆₉ | M1 | 33 | 1087834 | G | A | 3 | CED82350 |
| X₃₇₀ | M1 | 33 | 1127676 | G | A | 3 | CED82358 |
| X₃₇₁ | M1 | 33 | 1137501 | G | A | 2 | CED82362 |
| X₃₇₂ | M1 | 33 | 1138444 | C | T | 3 | CED82363 |
| X₃₇₃ | M1 | 33 | 1165914 | G | A | 2 | CED82372 |
| X₃₇₄ | M1 | 33 | 1169025 | C | T | 2 | CED82372 |
| X₃₇₅ | M1 | 33 | 1191727 | C | T | 2 | CED82379 |
| X₃₇₆ | M1 | 33 | 1324395 | G | A | 3 | CED82417 |
| X₃₇₇ | M1 | 33 | 1339376 | C | T | 2 | CED82424 |
| X₃₇₈ | M1 | 33 | 1367015 | C | T | 2 | CED82432 |
| X₃₇₉ | M1 | 33 | 1442014 | G | A | 2 | CED82456 |
| X₃₈₀ | M1 | 33 | 1467702 | C | T | 1 | CED82463 |
| X₃₈₁ | M1 | 33 | 1470375 | C | T | 8 | CED82465 |
| X₃₈₂ | M1 | 33 | 1489288 | G | A | 1 | CED82471 |
| X₃₈₃ | M1 | 33 | 1499347 | G | A | 3 | CED82472 |
| X₃₈₄ | M1 | 33 | 1515148 | G | A | 2 | CED82478 |
| X₃₈₅ | M1 | 33 | 1515369 | G | A | 3 | CED82478 |
| X₃₈₆ | M1 | 33 | 1515652 | C | T | 3 | CED82478 |
| X₃₈₇ | M1 | 33 | 1544767 | A | C | 6 | CED82486 |
| | | | | | | | CED82485- |
| X₃₈₈ | M1 | 33 | 1544951 | C | G | 6 | CED82486 |
| | | | | | | | CED82485- |
| X₃₈₉ | M1 | 33 | 1572498 | C | T | 8 | CED82491 |
| X₃₉₀ | M1 | 33 | 1587443 | C | T | 3 | CED82497 |
| X₃₉₁ | M1 | 33 | 1596242 | C | T | 3 | CED82498 |
| X₃₉₂ | M1 | 33 | 1638726 | G | A | 3 | CED82513 |
| X₃₉₃ | M1 | 33 | 1642659 | G | A | 2 | CED82515 |
| X₃₉₄ | M1 | 33 | 1652749 | C | T | 3 | CED82516 |
| X₃₉₅ | M1 | 33 | 1666106 | C | T | 8 | CED82522 |
| X₃₉₆ | M1 | 33 | 1682149 | C | T | 1 | CED82526 |
| X₃₉₇ | M1 | 33 | 1700000 | G | A | 2 | CED82534 |
| X₃₉₈ | M1 | 33 | 1795298 | G | A | 1 | CED82562 |
| X₃₉₉ | M1 | 33 | 1841263 | G | A | 2 | CED82576 |
| X₄₀₀ | M1 | 33 | 1860278 | G | A | 3 | CED82582 |
| X₄₀₁ | M1 | 33 | 1861675 | C | T | 2 | CED82583 |
| X₄₀₂ | M1 | 33 | 1862200 | G | A | 3 | CED82582 |
| X₄₀₃ | M1 | 33 | 1881896 | T | C | 3 | CED82588 |
| X₄₀₄ | M1 | 33 | 1887401 | C | T | 1 | CED82591 |
| X₄₀₅ | M1 | 33 | 1888706 | C | T | 8 | CED82592 |
| X₄₀₆ | M1 | 33 | 1914838 | C | T | 8 | CED82602 |
| X₄₀₇ | M1 | 33 | 1938614 | G | A | 2 | CED82612 |
| X₄₀₈ | M1 | 33 | 1959142 | G | A | 8 | CED82619 |
| X₄₀₉ | M1 | 33 | 2008504 | C | A | 1 | CED82635 |
| X₄₁₀ | M1 | 33 | 2010230 | C | T | 2 | CED82636 |
| X₄₁₁ | M1 | 33 | 2034962 | G | A | 2 | CED82645 |
| X₄₁₂ | M1 | 33 | 2044014 | G | A | 3 | CED82647 |
| X₄₁₃ | M1 | 33 | 2050847 | G | A | 3 | CED82651 |
| X₄₁₄ | M1 | 36 | 320 | G | A | 6 | |
| X₄₁₅ | M1 | 36 | 1056 | G | A | 6 | |
| X₄₁₆ | M1 | 36 | 1295 | C | T | 6 | |
| X₄₁₇ | M1 | 36 | 1461 | A | T | 6 | |
| X₄₁₈ | M1 | 42 | 804 | C | T | 4 | CDZ96333 |
| X₄₁₉ | M1 | 42 | 1559 | G | A | 4 | CDZ96333 |
| X₄₂₀ | M1 | 48 | 405 | G | A | 6 | |
| X₄₂₁ | M1 | 48 | 563 | C | T | 6 | |
| X₄₂₂ | M1 | 48 | 565 | T | C | 6 | |
| X₄₂₃ | M1 | 48 | 576 | G | A | 6 | |
| X₄₂₄ | M1 | 48 | 580 | T | A | 6 | |
| X₄₂₅ | M1 | 50 | 145 | G | A | 6 | |
| X₄₂₆ | M1 | 52 | 140940 | T | C | 4 | CED82705 |
| X₄₂₇ | M1 | 52 | 144382 | C | T | 3 | CED82709 |
| X₄₂₈ | M1 | 52 | 150382 | C | T | 1 | CED82712 |
| X₄₂₉ | M1 | 52 | 151974 | C | T | 2 | CED82712 |
| X₄₃₀ | M1 | 52 | 183986 | C | T | 8 | CED82722 |
| X₄₃₁ | M1 | 52 | 207354 | C | T | 8 | CED82730 |
| X₄₃₂ | M1 | 52 | 284733 | G | A | 1 | CED82747 |
| X₄₃₃ | M1 | 52 | 316848 | C | T | 2 | CED82758 |
| X₄₃₄ | M1 | 52 | 346628 | G | A | 2 | CED82769 |
| X₄₃₅ | M1 | 52 | 360279 | C | T | 3 | CED82772 |
| X₄₃₆ | M1 | 52 | 390095 | G | A | 3 | CED82784 |
| X₄₃₇ | M1 | 52 | 395374 | G | A | 2 | CED82786 |
| X₄₃₈ | M1 | 52 | 398723 | C | T | 3 | CED82786 |
| X₄₃₉ | M1 | 52 | 398728 | C | T | 3 | CED82786 |
| X₄₄₀ | M1 | 52 | 516267 | C | T | 3 | CED82823 |
| X₄₄₁ | M1 | 52 | 552099 | G | A | 2 | CED82837 |
| X₄₄₂ | M1 | 52 | 552173 | G | A | 1 | CED82837 |
| X₄₄₃ | M1 | 52 | 571124 | G | A | 2 | CED82845 |
| X₄₄₄ | M1 | 52 | 595806 | C | T | 1 | CED82851 |
| X₄₄₅ | M1 | 52 | 631479 | C | T | 1 | CED82860 |
| X₄₄₆ | M1 | 52 | 746548 | G | A | 1 | CED82907 |
| X₄₄₇ | M1 | 52 | 758326 | C | T | 2 | CED82912 |
| X₄₄₈ | M1 | 52 | 817452 | T | A | 2 | CED82928 |
| X₄₄₉ | M1 | 52 | 840755 | C | T | 1 | CED82935 |
| X₄₅₀ | M1 | 52 | 842352 | C | T | 3 | CED82934 |
| X₄₅₁ | M1 | 52 | 849671 | G | A | 1 | CED82940 |
| X₄₅₂ | M1 | 52 | 928854 | C | T | 2 | CED82971 |
| X₄₅₃ | M1 | 52 | 938650 | G | A | 3 | CED82973 |
| X₄₅₄ | M1 | 52 | 967316 | A | T | 2 | CED82984 |
| X₄₅₅ | M1 | 52 | 967701 | T | A | 3 | CED82983 |
| X₄₅₆ | M1 | 52 | 970142 | T | C | 3 | CED82985 |
| X₄₅₇ | M1 | 52 | 978727 | C | T | 3 | CED82988 |
| X₄₅₈ | M1 | 52 | 1003899 | G | A | 2 | CED83001 |
| X₄₅₉ | M1 | 52 | 1010006 | C | T | 2 | CED83002 |
| X₄₆₀ | M1 | 52 | 1029498 | G | A | 1 | CED83007 |
| X₄₆₁ | M1 | 52 | 1043011 | C | T | 1 | CED83012 |
| X₄₆₂ | M1 | 52 | 1067895 | C | T | 1 | CED83020 |
| X₄₆₃ | M1 | 52 | 1158103 | C | T | 3 | CED83051 |
| X₄₆₄ | M1 | 52 | 1207168 | C | T | 2 | CED83065 |
| X₄₆₅ | M1 | 52 | 1219894 | C | T | 3 | CED83071 |
| X₄₆₆ | M1 | 52 | 1290031 | C | T | 3 | CED83096 |
| X₄₆₇ | M1 | 52 | 1291646 | C | T | 2 | CED83098 |
| X₄₆₈ | M1 | 52 | 1363357 | G | A | 3 | CED83122 |
| X₄₆₉ | M1 | 52 | 1363405 | C | T | 1 | CED83123 |
| X₄₇₀ | M1 | 52 | 1379155 | G | A | 2 | CED83128 |
| X₄₇₁ | M1 | 52 | 1389320 | G | A | 3 | CED83130 |
| X₄₇₂ | M1 | 52 | 1399439 | G | A | 2 | CED83134 |
| X₄₇₃ | M1 | 52 | 1474315 | G | A | 2 | CED83164 |
| X₄₇₄ | M1 | 52 | 1474412 | G | A | 3 | CED83166 |
| X₄₇₅ | M1 | 52 | 1481345 | G | A | 3 | CED83165 |
| X₄₇₆ | M1 | 52 | 1520883 | C | T | 3 | CED83179 |
| X₄₇₇ | M1 | 52 | 1526796 | G | A | 2 | CED83181 |
| X₄₇₈ | M1 | 52 | 1583655 | C | T | 2 | CED83206 |
| X₄₇₉ | M1 | 52 | 1621166 | C | T | 2 | CED83219 |
| X₄₈₀ | M1 | 52 | 1639254 | C | T | 1 | CED83227 |
| X₄₈₁ | M1 | 52 | 1708900 | A | G | 3 | CED83249 |
| X₄₈₂ | M1 | 52 | 1715136 | C | T | 8 | CED83250 |
| X₄₈₃ | M1 | 52 | 1715899 | C | T | 2 | CED83250 |
| X₄₈₄ | M1 | 52 | 1716210 | C | T | 3 | CED83250 |
| X₄₈₅ | M1 | 52 | 1760440 | G | A | 2 | CED83266 |
| X₄₈₆ | M1 | 52 | 1785354 | T | C | 3 | CED83272 |
| X₄₈₇ | M1 | 52 | 1788070 | G | A | 4 | CED83273 |
| X₄₈₈ | M1 | 52 | 1793370 | G | A | 3 | CED83275 |
| X₄₈₉ | M1 | 52 | 1807309 | G | A | 3 | CED83283 |
| X₄₉₀ | M1 | 52 | 1809897 | G | A | 3 | CED83285 |
| X₄₉₁ | M1 | 52 | 1861665 | C | T | 2 | CED83301 |
| X₄₉₂ | M1 | 52 | 1861681 | G | A | 2 | CED83301 |
| X₄₉₃ | M1 | 52 | 1871310 | C | T | 2 | CED83304 |
| X₄₉₁ | M1 | 52 | 1874150 | G | T | 3 | CED83305 |
| X₄₉₅ | M1 | 52 | 1893352 | G | A | 2 | CED83313 |
| X₄₉₆ | M1 | 52 | 1963875 | G | A | 3 | CED83335 |
| X₄₉₇ | M1 | 52 | 1987096 | G | A | 4 | CED83341 |
| X₄₉₈ | M1 | 52 | 1995167 | G | A | 3 | CED83342 |
| X₄₉₉ | M1 | 52 | 2029893 | G | A | 3 | CED83351 |
| X₅₀₀ | M1 | 52 | 2041781 | C | T | 8 | CED83355 |
| X₅₀₁ | M1 | 52 | 2047954 | G | A | 2 | CED83358 |
| X₅₀₂ | M1 | 52 | 2048018 | G | A | 2 | CED83358 |
| X₅₀₃ | M1 | 52 | 2050245 | G | A | 3 | CED83359 |
| X₅₀₄ | M1 | 52 | 2071127 | C | T | 3 | CED83365 |
| X₅₀₅ | M1 | 52 | 2081390 | G | A | 1 | CED83367 |
| X₅₀₆ | M1 | 52 | 2093148 | C | T | 3 | CED83368 |
| X₅₀₇ | M1 | 52 | 2120665 | C | T | 4 | CED83376 |
| X₅₀₈ | M1 | 52 | 2136885 | A | G | 1 | CED83382 |
| X₅₀₉ | M1 | 52 | 2158892 | C | T | 3 | CED83392 |
| X₅₁₀ | M1 | 52 | 2162878 | G | A | 2 | CED83392 |
| X₅₁₁ | M1 | 52 | 2178340 | G | A | 8 | CED83397 |
| X₅₁₂ | M1 | 52 | 2265219 | C | T | 2 | CED83425 |
| X₅₁₃ | M1 | 52 | 2266378 | C | T | 3 | CED83423 |
| X₅₁₄ | M1 | 52 | 2308421 | T | C | 3 | CED83439 |
| X₅₁₅ | M1 | 52 | 2327266 | C | T | 3 | CED83445 |
| X₅₁₆ | M1 | 52 | 2330881 | G | A | 4 | CED83447 |
| X₅₁₇ | M1 | 52 | 2364315 | G | A | 4 | CED83455 |
| X₅₁₈ | M1 | 52 | 2400778 | C | T | 4 | CED83463 |
| X₅₁₉ | M1 | 52 | 2415283 | C | T | 2 | CED83469 |
| X₅₂₀ | M1 | 52 | 2469433 | C | T | 3 | CED83486 |
| X₅₂₁ | M1 | 52 | 2531584 | C | T | 2 | CED83507 |
| X₅₂₂ | M1 | 52 | 2566106 | C | T | 3 | CED83520 |
| X₅₂₃ | M1 | 52 | 2575090 | A | G | 3 | CED83525 |
| X₅₂₄ | M1 | 53 | 26291 | G | A | 2 | CDZ96348 |
| X₅₂₅ | M1 | 54 | 4815 | G | A | 1 | CDZ96384 |
| X₅₂₆ | M1 | 54 | 4816 | A | T | 2 | CDZ96384 |
| X₅₂₇ | M1 | 54 | 4817 | G | C | 2 | 2CDZ96384 |
| X₅₂₈ | M1 | 54 | 4821 | G | A | 1 | CDZ96384 |
| X₅₂₉ | M1 | 54 | 72235 | G | A | 2 | CDZ96406 |
| X₅₃₀ | M1 | 54 | 87947 | G | A | 3 | CDZ96410 |
| X₅₃₁ | M1 | 54 | 106086 | G | A | 3 | CDZ96416 |
| X₅₃₂ | M1 | 54 | 119439 | C | T | 3 | CDZ96420 |
| X₅₃₃ | M1 | 54 | 157787 | T | C | 1 | CDZ96435 |
| X₅₃₄ | M1 | 54 | 194230 | G | A | 2 | CDZ96446 |
| X₅₃₅ | M1 | 54 | 202467 | C | T | 1 | CDZ96448 |
| X₅₃₆ | M1 | 54 | 206494 | G | A | 2 | CDZ96450 |
| X₅₃₇ | M1 | 54 | 254295 | C | T | 2 | CDZ96465 |
| X₅₃₈ | M1 | 54 | 274617 | A | T | 3 | CDZ96471 |
| X₅₃₉ | M1 | 54 | 357149 | G | A | 1 | CDZ96496 |
| X₅₄₀ | M1 | 54 | 359671 | G | A | 2 | CDZ96496 |
| X₅₄₁ | M1 | 54 | 370320 | G | A | 2 | CDZ96499 |
| X₅₄₂ | M1 | 54 | 391223 | A | G | 2 | CDZ96509 |
| X₅₄₃ | M1 | 54 | 410031 | C | A | 1 | CDZ96514 |
| X₅₄₄ | M1 | 54 | 427542 | C | T | 2 | CDZ96520 |
| X₅₄₅ | M1 | 54 | 431851 | C | T | 3 | CDZ96520 |
| X₅₄₆ | M1 | 54 | 437080 | G | A | 3 | CDZ96522 |
| X₅₄₇ | M1 | 54 | 478527 | C | T | 2 | CDZ96537 |
| X₅₄₈ | M1 | 54 | 564688 | C | T | 2 | CDZ96566 |
| X₅₄₉ | M1 | 54 | 576174 | G | A | 2 | CDZ96569 |
| X₅₅₀ | M1 | 54 | 583996 | G | A | 3 | CDZ96570 |
| X₅₅₁ | M1 | 54 | 616268 | A | G | 3 | CDZ96581 |
| X₅₅₂ | M1 | 54 | 647579 | C | T | 2 | CDZ96590 |
| X₅₅₃ | M1 | 54 | 667019 | C | T | 4 | CDZ96594 |
| X₅₅₄ | M1 | 54 | 667042 | C | T | 8 | CDZ96595 |
| X₅₅₅ | M1 | 54 | 733193 | T | C | 9 | CDZ96619 |
| X₅₅₆ | M1 | 54 | 743396 | G | A | 1 | CDZ96621 |
| X₅₅₇ | M1 | 58 | 93 | T | C | 6 | |
| X₅₅₈ | M1 | 67 | 111 | G | A | 6 | |
| X₅₅₉ | M1 | 69 | 29006 | G | A | 3 | CED83534 |
| X₅₆₀ | M1 | 69 | 34392 | G | A | 2 | CED83536 |
| X₅₆₁ | M1 | 69 | 99248 | C | T | 2 | CED83559 |
| X₅₆₂ | M1 | 69 | 107920 | C | T | 1 | CED83561 |
| X₅₆₃ | M1 | 69 | 201005 | G | A | 3 | CED83595 |
| X₅₆₄ | M1 | 69 | 293602 | G | A | 3 | CED83626 |
| X₅₆₅ | M1 | 69 | 305997 | C | T | 2 | CED83631 |
| X₅₆₆ | M1 | 69 | 371916 | C | T | 2 | CED83653 |
| X₅₆₇ | M1 | 69 | 415659 | C | T | 2 | CED83665 |
| X₅₆₈ | M1 | 69 | 461021 | C | T | 2 | CED83680 |
| X₅₆₉ | M1 | 69 | 461986 | G | A | 1 | CED83680 |
| X₅₇₀ | M1 | 69 | 472563 | C | T | 3 | CED83681 |
| X₅₇₁ | M1 | 69 | 482073 | G | T | 3 | CED83685 |
| X₅₇₂ | M1 | 69 | 556138 | C | T | 3 | CED83710 |
| X₅₇₃ | M1 | 69 | 585845 | G | A | 2 | CED83722 |
| X₅₇₄ | M1 | 69 | 627928 | C | T | 1 | CED83740 |
| X₅₇₅ | M1 | 69 | 631511 | G | A | 2 | CED83742 |
| X₅₇₆ | M1 | 69 | 637299 | T | C | 2 | CED83744 |
| X₅₇₇ | M1 | 69 | 675579 | G | A | 3 | CED83760 |
| X₅₇₈ | M1 | 69 | 676581 | G | A | 2 | CED83760 |
| X₅₇₉ | M1 | 69 | 676853 | G | A | 3 | CED83759 |
| X₅₈₀ | M1 | 69 | 678406 | C | T | 3 | CED83759 |
| X₅₈₁ | M1 | 69 | 757546 | C | A | 2 | CED83788 |
| X₅₈₂ | M1 | 69 | 828676 | C | T | 2 | CED83805 |
| X₅₈₃ | M1 | 69 | 857539 | C | T | 3 | CED83814 |
| X₅₈₄ | M1 | 69 | 865820 | C | T | 2 | CED83814 |
| X₅₈₅ | M1 | 69 | 869706 | G | A | 8 | CED83815 |
| X₅₈₆ | M1 | 69 | 892199 | G | A | 1 | CED83822 |
| X₅₈₇ | M1 | 69 | 894462 | C | T | 3 | CED83821 |
| X₅₈₈ | M1 | 69 | 898044 | C | T | 1 | CED83823 |
| X₅₈₉ | M1 | 69 | 962369 | G | A | 3 | CED83845 |
| X₅₉₀ | M1 | 69 | 971219 | C | T | 3 | CED83847 |
| X₅₉₁ | M1 | 69 | 988364 | G | A | 2 | CED83857 |
| X₅₉₂ | M1 | 69 | 989781 | G | A | 2 | CED83858 |
| X₅₉₃ | M1 | 69 | 1042212 | G | A | 2 | CED83875 |
| X₅₉₄ | M1 | 69 | 1080578 | G | A | 2 | CED83890 |
| X₅₉₅ | M1 | 69 | 1084824 | G | A | 3 | CED83891 |
| X₅₉₆ | M1 | 69 | 1084835 | C | T | 3 | CED83891 |
| X₅₉₇ | M1 | 69 | 1084848 | A | G | 3 | CED83891 |
| X₅₉₈ | M1 | 69 | 1084866 | A | G | 3 | CED83891 |
| X₅₉₉ | M1 | 69 | 1084931 | C | T | 3 | CED83891 |
| X₆₀₀ | M1 | 69 | 1102164 | C | T | 2 | CED83897 |
| X₆₀₁ | M1 | 69 | 1105560 | C | T | 2 | CED83899 |
| X₆₀₂ | M1 | 69 | 1109785 | C | T | 2 | CED83899 |
| X₆₀₃ | M1 | 69 | 1109813 | A | G | 3 | CED83899 |
| X₆₀₄ | M1 | 69 | 1109818 | A | G | 3 | CED83899 |
| X₆₀₅ | M1 | 69 | 1109819 | T | A | 3 | CED83899 |
| X₆₀₆ | M1 | 69 | 1109823 | T | C | 3 | CED83899 |
| X₆₀₇ | M1 | 69 | 1109830 | A | G | 3 | CED83899 |
| X₆₀₈ | M1 | 69 | 1109831 | C | A | 3 | CED83899 |
| X₆₀₉ | M1 | 69 | 1109835 | A | G | 3 | CED83899 |
| X₆₁₀ | M1 | 69 | 1115486 | C | T | 2 | CED83902 |
| X₆₁₁ | M1 | 69 | 1153118 | G | A | 2 | CED83915 |
| X₆₁₂ | M1 | 69 | 1215253 | G | A | 1 | CED83936 |
| X₆₁₃ | M1 | 69 | 1218409 | G | A | 3 | CED83936 |
| X₆₁₄ | M1 | 69 | 1260087 | G | A | 3 | CED83949 |
| X₆₁₅ | M1 | 69 | 1279557 | G | A | 2 | CED83959 |
| X₆₁₆ | M1 | 69 | 1342909 | G | A | 3 | CED83975 |
| X₆₁₇ | M1 | 69 | 1356844 | C | T | 8 | CED83982 |
| X₆₁₈ | M1 | 69 | 1372486 | C | T | 3 | CED83987 |
| X₆₁₉ | M1 | 69 | 1377243 | C | T | 3 | CED83989 |
| X₆₂₀ | M1 | 69 | 1396045 | G | A | 2 | CED83994 |
| X₆₂₁ | M1 | 69 | 1411237 | C | T | 1 | CED84001 |
| X₆₂₂ | M1 | 69 | 1411421 | C | T | 3 | CED83999 |
| X₆₂₃ | M1 | 69 | 1530586 | C | T | 3 | CED84038 |
| X₆₂₄ | M1 | 69 | 1590302 | G | A | 1 | CED84059 |
| X₆₂₅ | M1 | 69 | 1617492 | A | G | 2 | CED84068 |
| X₆₂₆ | M1 | 69 | 1652288 | C | T | 3 | CED84077 |
| X₆₂₇ | M1 | 69 | 1674261 | C | T | 1 | CED84084 |
| X₆₂₈ | M1 | 69 | 1678475 | C | T | 2 | CED84085 |
| X₆₂₉ | M1 | 69 | 1687394 | G | A | 1 | CED84087 |
| X₆₃₀ | M1 | 69 | 1688555 | C | T | 1 | CED84088 |
| X₆₃₁ | M1 | 69 | 1701017 | G | A | 2 | CED84094 |
| X₆₃₂ | M1 | 69 | 1714728 | G | A | 3 | CED84097 |
| X₆₃₃ | M1 | 69 | 1750423 | C | T | 1 | CED84111 |
| X₆₃₄ | M1 | 69 | 1788929 | G | A | 2 | CED84124 |
| X₆₃₅ | M1 | 69 | 1790823 | C | T | 2 | CED84125 |
| X₆₃₆ | M1 | 69 | 1792597 | C | T | 2 | CED84126 |
| X₆₃₇ | M1 | 69 | 1799816 | C | T | 8 | CED84128 |
| X₆₃₈ | M1 | 69 | 1800584 | G | A | 3 | CED84129 |
| X₆₃₉ | M1 | 69 | 1854739 | G | A | 3 | CED84145 |
| X₆₄₀ | M1 | 69 | 1855951 | C | T | 3 | CED84145 |
| X₆₄₁ | M1 | 69 | 1871563 | G | A | 2 | CED84153 |
| X₆₄₂ | M1 | 69 | 1878663 | C | T | 3 | CED84154 |
| X₆₄₃ | M1 | 69 | 1924621 | C | T | 3 | CED84172 |
| X₆₄₄ | M1 | 69 | 1949938 | G | A | 3 | CED84182 |
| X₆₄₅ | M1 | 69 | 1967529 | C | T | 1 | CED84189 |
| X₆₄₆ | M1 | 69 | 1968848 | A | G | 3 | CED84189 |
| X₆₄₇ | M1 | 69 | 2001469 | C | T | 3 | CED84197 |
| X₆₄₈ | M1 | 69 | 2009058 | G | A | 8 | CED84201 |
| X₆₄₉ | M1 | 69 | 2031440 | G | A | 2 | CED84210 |
| X₆₅₀ | M1 | 69 | 2099452 | C | G | 4 | CED84233 |
| X₆₅₁ | M1 | 69 | 2099556 | C | T | 4 | CED84233 |
| X₆₅₂ | M1 | 69 | 2099559 | A | T | 4 | CED84233 |
| X₆₅₃ | M1 | 69 | 2160207 | C | T | 1 | CED84255 |
| X₆₅₄ | M1 | 69 | 2219444 | G | A | 2 | CED84271 |
| X₆₅₅ | M1 | 69 | 2226317 | C | T | 2 | CED84272 |
| X₆₅₆ | M1 | 69 | 2231926 | G | A | 2 | CED84274 |
| X₆₅₇ | M1 | 69 | 2237950 | C | T | 2 | CED84276 |
| X₆₅₈ | M1 | 69 | 2263333 | G | A | 2 | CED84285 |
| X₆₅₉ | M1 | 69 | 2275703 | G | A | 2 | CED84290 |
| X₆₆₀ | M1 | 69 | 2320079 | T | A | 3 | CED84303 |
| X₆₆₁ | M1 | 69 | 2402826 | C | T | 2 | CED84333 |
| X₆₆₂ | M1 | 69 | 2421550 | C | T | 2 | CED84338 |
| X₆₆₃ | M1 | 69 | 2438727 | C | T | 3 | CED84343 |
| X₆₆₄ | M1 | 69 | 2483433 | G | A | 3 | CED84352 |
| X₆₆₅ | M1 | 72 | 93 | T | C | 6 | |
| X₆₆₆ | M1 | 72 | 584 | G | A | 6 | |
| X₆₆₇ | M1 | 72 | 856 | C | T | 6 | |
| X₆₆₈ | M1 | 72 | 1163 | C | T | 6 | |
| X₆₆₉ | M1 | 73 | 928 | C | T | 4 | CDZ96628 |
| X₆₇₀ | M1 | 77 | 18912 | A | G | 2 | CDZ96635 |
| X₆₇₁ | M1 | 77 | 39842 | G | A | 8 | CDZ96646 |
| X₆₇₂ | M1 | 77 | 47151 | C | T | 2 | CDZ96648 |
| X₆₇₃ | M1 | 77 | 84022 | G | A | 8 | CDZ96659 |
| X₆₇₄ | M1 | 77 | 116691 | G | A | 3 | CDZ96671 |
| X₆₇₅ | M1 | 78 | 6333 | G | A | 8 | CED84360 |
| X₆₇₆ | M1 | 78 | 15591 | C | T | 3 | CED84363 |
| X₆₇₇ | M1 | 78 | 26915 | C | T | 3 | CED84366 |
| X₆₇₈ | M1 | 78 | 35200 | G | A | 2 | CED84371 |
| X₆₇₉ | M1 | 78 | 100169 | G | A | 2 | CED84388 |
| X₆₈₀ | M1 | 78 | 158434 | G | A | 2 | CED84406 |
| X₆₈₁ | M1 | 78 | 158534 | G | A | 2 | CED84406 |
| X₆₈₂ | M1 | 78 | 191358 | G | A | 3 | CED84414 |
| X₆₈₃ | M1 | 78 | 200234 | C | T | 2 | CED84420 |
| X₆₈₄ | M1 | 78 | 238854 | C | T | 3 | CED84430 |
| X₆₈₅ | M1 | 78 | 262614 | A | C | 2 | CED84438 |
| X₆₈₆ | M1 | 78 | 263828 | T | C | 3 | CED84438 |
| X₆₈₇ | M1 | 78 | 263896 | T | C | 3 | CED84438 |
| X₆₈₈ | M1 | 78 | 281454 | C | A | 3 | CED84441 |
| X₆₈₉ | M1 | 78 | 292068 | C | T | 1 | CED84442 |
| X₆₉₀ | M1 | 78 | 298141 | C | G | 3 | CED84443 |
| X₆₉₁ | M1 | 78 | 299166 | G | A | 3 | CED84443 |
| X₆₉₂ | M1 | 78 | 299644 | G | A | 3 | CED84443 |
| X₆₉₃ | M1 | 78 | 302700 | A | G | 2 | CED84443 |
| X₆₉₄ | M1 | 78 | 311586 | C | T | 2 | CED84447 |
| X₆₉₅ | M1 | 78 | 333312 | A | G | 3 | CED84452 |
| X₆₉₆ | M1 | 78 | 376706 | C | T | 3 | CED84464 |
| X₆₉₇ | M1 | 78 | 386356 | C | T | 2 | CED84472 |
| X₆₉₈ | M1 | 78 | 439640 | C | T | 2 | CED84487 |
| X₆₉₉ | M1 | 78 | 480244 | C | T | 1 | CED84502 |
| X₇₀₀ | M1 | 78 | 487303 | C | T | 3 | CED84505 |
| X₇₀₁ | M1 | 78 | 611362 | C | T | 2 | CED84546 |
| X₇₀₂ | M1 | 78 | 626958 | C | A | 4 | CED84549 |
| X₇₀₃ | M1 | 78 | 666005 | C | T | 3 | CED84562 |
| X₇₀₄ | M1 | 78 | 706821 | A | G | 3 | CED84578 |
| X₇₀₅ | M1 | 78 | 708376 | C | T | 2 | CED84580 |
| X₇₀₆ | M1 | 78 | 710966 | G | A | 2 | CED84581 |
| X₇₀₇ | M1 | 78 | 726983 | G | A | 2 | CED84587 |
| X₇₀₈ | M1 | 78 | 763771 | G | A | 3 | CED84600 |
| X₇₀₉ | M1 | 78 | 774252 | G | A | 2 | CED84602 |
| X₇₁₀ | M1 | 78 | 811274 | G | A | 2 | CED84614 |
| X₇₁₁ | M1 | 78 | 895505 | C | T | 2 | CED84642 |
| X₇₁₂ | M1 | 78 | 918865 | C | T | 1 | CED84651 |
| X₇₁₃ | M1 | 78 | 952815 | G | A | 2 | CED84662 |
| X₇₁₄ | M1 | 78 | 952860 | G | A | 2 | CED84662 |
| X₇₁₅ | M1 | 78 | 976799 | C | T | 3 | CED84672 |
| X₇₁₆ | M1 | 78 | 1023381 | G | A | 2 | CED84688 |
| X₇₁₇ | M1 | 78 | 1066463 | C | T | 2 | CED84704 |
| X₇₁₈ | M1 | 78 | 1095322 | C | T | 2 | CED84715 |
| X₇₁₉ | M1 | 78 | 1100041 | G | A | 3 | CED84715 |
| X₇₂₀ | M1 | 78 | 1112587 | C | T | 2 | CED84722 |
| X₇₂₁ | M1 | 78 | 1115511 | G | A | 1 | CED84723 |
| X₇₂₂ | M1 | 78 | 1142438 | G | A | 2 | CED84729 |
| X₇₂₃ | M1 | 78 | 1216573 | C | T | 1 | CED84752 |
| X₇₂₄ | M1 | 78 | 1266077 | G | A | 2 | CED84770 |
| X₇₂₅ | M1 | 78 | 1302286 | C | T | 3 | CED84782 |
| X₇₂₆ | M1 | 78 | 1315312 | C | T | 3 | CED84786 |
| X₇₂₇ | M1 | 78 | 1317124 | G | A | 3 | CED84787 |
| X₇₂₈ | M1 | 78 | 1322274 | G | A | 1 | CED84790 |
| X₇₂₉ | M1 | 78 | 1324423 | T | C | 2 | CED84791 |
| X₇₃₀ | M1 | 78 | 1344640 | C | T | 3 | CED84795 |
| X₇₃₁ | M1 | 78 | 1397982 | G | A | 3 | CED84818 |
| X₇₃₂ | M1 | 78 | 1426722 | C | T | 2 | CED84829 |
| X₇₃₃ | M1 | 78 | 1495995 | C | T | 1 | CED84852 |
| X₇₃₄ | M1 | 78 | 1520586 | C | T | 1 | CED84855 |
| X₇₃₅ | M1 | 78 | 1563246 | C | T | 3 | CED84865 |
| X₇₃₆ | M1 | 78 | 1568326 | C | T | 2 | CED84868 |
| X₇₃₇ | M1 | 78 | 1642170 | G | A | 3 | CED84889 |
| X₇₃₈ | M1 | 78 | 1660408 | G | A | 2 | CED84897 |
| X₇₃₉ | M1 | 78 | 1730707 | C | T | 3 | CED84922 |
| X₇₄₀ | M1 | 78 | 1742796 | G | A | 3 | CED84926 |
| X₇₄₁ | M1 | 78 | 1745162 | C | T | 4 | CED84927 |
| X₇₄₂ | M1 | 79 | 65 | G | A | 3 | CDZ96674 |
| X₇₄₃ | M1 | 79 | 133 | A | G | 3 | CDZ96674 |
| X₇₄₄ | M1 | 79 | 3698 | C | G | 4 | CDZ96674 |
| X₇₄₅ | M1 | 79 | 20544 | A | G | 2 | CDZ96675 |
| X₇₄₆ | M1 | 79 | 57804 | G | A | 2 | CDZ96687 |
| X₇₄₇ | M1 | 79 | 64133 | C | T | 3 | CDZ96688 |
| X₇₄₈ | M1 | 79 | 64658 | C | T | 3 | CDZ96688 |
| X₇₄₉ | M1 | 79 | 65402 | C | T | 3 | CDZ96688 |
| X₇₅₀ | M1 | 79 | 132683 | C | T | 2 | CDZ96713 |
| X₇₅₁ | M1 | 79 | 171814 | C | T | 3 | CDZ96727 |
| X₇₅₂ | M1 | 79 | 307021 | C | T | 3 | CDZ96777 |
| X₇₅₃ | M1 | 79 | 346823 | G | A | 2 | CDZ96789 |
| X₇₅₄ | M1 | 79 | 378247 | C | T | 2 | CDZ96800 |
| X₇₅₅ | M1 | 79 | 409085 | C | T | 8 | CDZ96810 |
| X₇₅₆ | M1 | 79 | 412661 | G | A | 2 | CDZ96811 |
| X₇₅₇ | M1 | 79 | 492523 | C | T | 3 | CDZ96842 |
| X₇₅₈ | M1 | 79 | 508274 | C | T | 8 | CDZ96848 |
| X₇₅₉ | M1 | 79 | 575411 | C | T | 3 | CDZ96868 |
| X₇₆₀ | M1 | 79 | 579674 | G | A | 1 | CDZ96871 |
| X₇₆₁ | M1 | 79 | 617057 | C | T | 2 | CDZ96882 |
| X₇₆₂ | M1 | 79 | 679140 | C | T | 2 | CDZ96904 |
| X₇₆₃ | M1 | 79 | 696174 | G | A | 4 | CDZ96911 |
| X₇₆₄ | M1 | 79 | 710422 | G | A | 2 | CDZ96916 |
| X₇₆₅ | M1 | 79 | 735053 | T | C | 2 | CDZ96928 |
| X₇₆₆ | M1 | 79 | 749596 | G | A | 4 | CDZ96930 |
| X₇₆₇ | M1 | 79 | 759403 | C | T | 3 | CDZ96931 |
| X₇₆₈ | M1 | 79 | 759659 | G | A | 2 | CDZ96931 |
| X₇₆₉ | M1 | 79 | 760458 | G | T | 1 | CDZ96932 |
| X₇₇₀ | M1 | 79 | 760541 | A | G | 2 | CDZ96932 |
| X₇₇₁ | M1 | 79 | 760891 | A | G | 2 | CDZ96932 |
| X₇₇₂ | M1 | 79 | 760904 | T | C | 2 | CDZ96932 |
| X₇₇₃ | M1 | 79 | 761056 | T | C | 3 | CDZ96932 |
| X₇₇₄ | M1 | 79 | 768537 | A | G | 4 | CDZ96933 |
| X₇₇₅ | M1 | 79 | 776112 | A | G | 4 | CDZ96934 |
| X₇₇₆ | M1 | 79 | 786064 | A | G | 3 | CDZ96934 |
| X₇₇₇ | M1 | 79 | 798799 | C | T | 1 | CDZ96939 |
| X₇₇₈ | M1 | 79 | 914302 | C | T | 3 | CDZ96977 |
| X₇₇₉ | M1 | 79 | 940003 | C | T | 2 | CDZ96991 |
| X₇₈₀ | M1 | 79 | 962607 | G | A | 3 | CDZ96997 |
| X₇₈₁ | M1 | 79 | 1000786 | A | G | 1 | CDZ97007 |
| X₇₈₂ | M1 | 79 | 1002991 | G | A | 1 | CDZ97008 |
| X₇₈₃ | M1 | 79 | 1044652 | G | A | 1 | CDZ97023 |
| X₇₈₄ | M1 | 79 | 1056822 | G | T | 3 | CDZ97025 |
| X₇₈₅ | M1 | 79 | 1069426 | C | T | 2 | CDZ97032 |
| X₇₈₆ | M1 | 79 | 1075960 | G | A | 8 | CDZ97034 |
| X₇₈₇ | M1 | 79 | 1086267 | G | A | 2 | CDZ97038 |
| X₇₈₈ | M1 | 79 | 1123288 | C | T | 2 | CDZ97049 |
| X₇₈₉ | M1 | 79 | 1140763 | G | A | 3 | CDZ97053 |
| X₇₉₀ | M1 | 79 | 1191202 | G | A | 3 | CDZ97070 |
| X₇₉₁ | M1 | 79 | 1229129 | G | A | 2 | CDZ97086 |
| X₇₉₂ | M1 | 79 | 1231740 | C | T | 2 | CDZ97087 |
| X₇₉₃ | M1 | 79 | 1265502 | C | T | 1 | CDZ97100 |
| X₇₉₄ | M1 | 79 | 1284476 | C | T | 1 | CDZ97106 |
| X₇₉₅ | M1 | 79 | 1318034 | C | T | 1 | CDZ97117 |
| X₇₉₆ | M1 | 79 | 1336766 | C | T | 4 | CDZ97121 |
| X₇₉₇ | M1 | 79 | 1344403 | C | T | 2 | CDZ97124 |
| X₇₉₈ | M1 | 79 | 1367232 | C | T | 5 | CDZ97129 |
| X₇₉₉ | M1 | 79 | 1387800 | G | A | 3 | CDZ97135 |
| X₈₀₀ | M1 | 79 | 1410287 | C | T | 1 | CDZ97145 |
| X₈₀₁ | M1 | 79 | 1410481 | G | A | 1 | CDZ97145 |
| X₈₀₂ | M1 | 79 | 1423385 | C | T | 1 | CDZ97150 |
| X₈₀₃ | M1 | 79 | 1430954 | G | A | 2 | CDZ97152 |
| X₈₀₄ | M1 | 79 | 1471094 | C | T | 3 | CDZ97163 |
| X₈₀₅ | M1 | 79 | 1540205 | C | T | 2 | CDZ97186 |
| X₈₀₆ | M1 | 79 | 1564902 | C | T | 1 | CDZ97195 |
| X₈₀₇ | M1 | 79 | 1624415 | G | A | 1 | CDZ97217 |
| X₈₀₈ | M1 | 79 | 1674448 | G | A | 4 | CDZ97228 |
| X₈₀₉ | M1 | 79 | 1732022 | G | A | 3 | CDZ97245 |
| X₈₁₀ | M1 | 79 | 1797660 | C | T | 3 | CDZ97265 |
| X₈₁₁ | M1 | 79 | 1797676 | G | A | 3 | CDZ97265 |
| X₈₁₂ | M1 | 79 | 1842448 | G | A | 2 | CDZ97282 |
| X₈₁₃ | M1 | 79 | 1854359 | G | A | 3 | CDZ97285 |
| X₈₁₄ | M1 | 79 | 1855037 | A | G | 3 | CDZ97285 |
| X₈₁₅ | M1 | 79 | 1859220 | A | G | 1 | CDZ97291 |
| X₈₁₆ | M1 | 79 | 1883004 | A | G | 1 | CDZ97298 |
| X₈₁₇ | M1 | 79 | 1883021 | C | A | 2 | CDZ97298 |
| X₈₁₈ | M1 | 79 | 1930688 | G | A | 3 | CDZ97315 |
| X₈₁₉ | M1 | 79 | 1934434 | G | A | 3 | CDZ97316 |
| X₈₂₀ | M1 | 79 | 1977783 | G | A | 3 | CDZ97330 |
| X₈₂₁ | M1 | 79 | 1990424 | G | A | 2 | CDZ97334 |
| X₈₂₂ | M1 | 79 | 2013304 | G | A | 2 | CDZ97344 |
| X₈₂₃ | M1 | 79 | 2013361 | C | T | 8 | CDZ97344 |
| X₈₂₄ | M1 | 79 | 2045917 | C | T | 3 | CDZ97355 |
| X₈₂₅ | M1 | 79 | 2052320 | C | T | 2 | CDZ97361 |
| X₈₂₆ | M1 | 79 | 2073260 | G | T | 3 | CDZ97366 |
| X₈₂₇ | M1 | 79 | 2089423 | C | T | 2 | CDZ97372 |
| X₈₂₈ | M1 | 79 | 2113064 | G | A | 3 | CDZ97381 |
| X₈₂₉ | M1 | 79 | 2121041 | G | A | 3 | CDZ97380 |
| X₈₃₀ | M1 | 79 | 2152573 | C | T | 2 | CDZ97392 |
| X₈₃₁ | M1 | 79 | 2169813 | C | T | 3 | CDZ97396 |
| X₈₃₂ | M1 | 79 | 2172085 | C | T | 3 | CDZ97397 |
| X₈₃₃ | M1 | 79 | 2179936 | G | A | 1 | CDZ97402 |
| X₈₃₄ | M1 | 79 | 2187306 | C | T | 3 | CDZ97404 |
| X₈₃₅ | M1 | 79 | 2188410 | G | A | 2 | CDZ97405 |
| X₈₃₆ | M1 | 79 | 2220254 | C | T | 3 | CDZ97414 |
| X₈₃₇ | M1 | 79 | 2268540 | C | T | 1 | CDZ97435 |
| X₈₃₈ | M1 | 79 | 2271652 | G | C | 2 | CDZ97437 |
| X₈₃₉ | M1 | 79 | 2294761 | G | A | 8 | CDZ97444 |
| X₈₄₀ | M1 | 79 | 2320060 | G | A | 2 | CDZ97454 |
| X₈₄₁ | M1 | 79 | 2339881 | C | G | 3 | CDZ97460 |
| X₈₄₂ | M1 | 79 | 2340326 | G | A | 3 | CDZ97462 |
| X₈₄₃ | M1 | 85 | 462 | G | A | 6 | |
| X₈₄₄ | M1 | 85 | 463 | G | G | 6 | |
| X₈₄₅ | M1 | 85 | 467 | A | T | 6 | |
| X₈₄₆ | M1 | 85 | 512 | A | G | 6 | |
| X₈₄₇ | M1 | 85 | 516 | G | A | 6 | |
| X₈₄₈ | M1 | 85 | 521 | C | T | 6 | |
| X₈₄₉ | M1 | 85 | 522 | A | G | 6 | |
| X₈₅₀ | M1 | 86 | 230 | A | G | 6 | |
| X₈₅₁ | M1 | 88 | 119 | C | T | 3 | CDZ97464 |
| X₈₅₂ | M1 | 88 | 196 | G | A | 3 | CDZ97464 |
| X₈₅₃ | M1 | 88 | 898 | C | T | 4 | CDZ97464 |
| X₈₅₄ | M1 | 99 | 245 | G | A | 6 | |
| X₈₅₅ | M1 | 105 | 258 | A | G | 2 | CDZ97474 |
| X₈₅₆ | M1 | 105 | 429 | C | T | 4 | CDZ97474 |
| X₈₅₇ | M1 | 106 | 278 | A | G | 6 | |
| X₈₅₈ | M1 | 109 | 396 | T | C | 6 | |
| X₈₅₉ | M1 | 115 | 361 | G | A | 6 | |
| X₈₆₀ | M1 | 118 | 396 | C | T | 2 | CDZ97478 |
| X₈₆₁ | M1 | 118 | 870 | G | A | 4 | CDZ97478 |
| X₈₆₂ | M1 | 118 | 1491 | G | A | 4 | CDZ97478 |
| X₈₆₃ | M1 | 124 | 72117 | G | A | 2 | CDZ97501 |
| X₈₆₄ | M1 | 124 | 72760 | G | A | 1 | CDZ97501 |
| X₈₆₅ | M1 | 124 | 94611 | G | A | 1 | CDZ97507 |
| X₈₆₆ | M1 | 124 | 156052 | G | A | 2 | CDZ97524 |
| X₈₆₇ | M1 | 124 | 171458 | G | T | 1 | CDZ97530 |
| X₈₆₈ | M1 | 124 | 179336 | C | T | 1 | CDZ97533 |
| X₈₆₉ | M1 | 124 | 207992 | C | T | 3 | CDZ97541 |
| X₈₇₀ | M1 | 124 | 222249 | C | T | 1 | CDZ97547 |
| X₈₇₁ | M1 | 125 | 35864 | T | C | 3 | CDZ97573 |
| X₈₇₂ | M1 | 129 | 299 | G | A | 3 | CDZ97577 |
| X₈₇₃ | M1 | 129 | 433 | G | A | 3 | CDZ97577 |
| X₈₇₄ | M1 | 129 | 856 | G | A | 2 | CDZ97577 |
| X₈₇₅ | M1 | 129 | 1013 | C | T | 1 | CDZ97577 |
| X₈₇₆ | M1 | 130 | 674 | C | T | 4 | CDZ97578 |
| X₈₇₇ | M1 | 138 | 123 | G | T | 6 | |
| X₈₇₈ | M1 | 141 | 15764 | C | T | 2 | CDZ97587 |
| X₈₇₉ | M1 | 141 | 25126 | C | T | 2 | CDZ97591 |
| X₈₈₀ | M1 | 149 | 1663 | A | G | 3 | CDZ97598 |
| X₈₈₁ | M1 | 149 | 2074 | T | G | 3 | CDZ97598 |
| X₈₈₂ | M1 | 149 | 2453 | C | T | 3 | CDZ97598 |
| X₈₈₃ | M1 | 159 | 106 | A | C | 6 | |
| X₈₈₄ | M1 | 162 | 47699 | G | A | 2 | CDZ97619 |
| X₈₈₅ | M1 | 162 | 70405 | A | G | 1 | CDZ97625 |
| X₈₈₆ | M1 | 162 | 148101 | G | A | 3 | CDZ97648 |
| X₈₈₇ | M1 | 162 | 207179 | C | T | 8 | CDZ97668 |
| X₈₈₈ | M1 | 162 | 209584 | C | T | 3 | CDZ97670 |
| X₈₈₉ | M1 | 162 | 223407 | C | T | 2 | CDZ97674 |
| X₈₉₀ | M1 | 162 | 227242 | G | A | 3 | CDZ97674 |
| X₈₉₁ | M1 | 162 | 233092 | C | T | 3 | CDZ97676 |
| X₈₉₂ | M1 | 162 | 291566 | G | A | 4 | CDZ97695 |
| X₈₉₃ | M1 | 162 | 308369 | T | C | 1 | CDZ97702 |
| X₈₉₄ | M1 | 162 | 318857 | G | A | 4 | CDZ97703 |
| X₈₉₅ | M1 | 162 | 320785 | G | C | 4 | CDZ97703 |
| X₈₉₆ | M1 | 162 | 320786 | T | G | 4 | CDZ97703 |
| X₈₉₇ | M1 | 162 | 321460 | C | G | 4 | CDZ97703 |
| X₈₉₈ | M1 | 162 | 321463 | A | C | 4 | CDZ97703 |
| X₈₉₉ | M1 | 162 | 322591 | G | A | 4 | CDZ97703 |
| X₉₀₀ | M1 | 162 | 323448 | T | G | 2 | CDZ97703 |
| X₉₀₁ | M1 | 162 | 323451 | T | G | 2 | CDZ97703 |
| X₉₀₂ | M1 | 162 | 323480 | T | C | 1 | CDZ97703 |
| X₉₀₃ | M1 | 162 | 323735 | A | G | 1 | CDZ97703 |
| X₉₀₄ | M1 | 162 | 324114 | T | C | 2 | CDZ97703 |
| X₉₀₅ | M1 | 162 | 324139 | C | T | 2 | CDZ97703 |
| X₉₀₆ | M1 | 162 | 324568 | A | G | 2 | CDZ97703 |
| X₉₀₇ | M1 | 162 | 360320 | G | A | 2 | CDZ97714 |
| X₉₀₈ | M1 | 162 | 398165 | C | T | 1 | CDZ97730 |
| X₉₀₉ | M1 | 162 | 414579 | C | T | 8 | CDZ97737 |
| X₉₁₀ | M1 | 162 | 424391 | C | T | 3 | CDZ97740 |
| X₉₁₁ | M1 | 162 | 444123 | T | C | 2 | CDZ97748 |
| X₉₁₂ | M1 | 162 | 467207 | C | T | 3 | CDZ97755 |
| X₉₁₃ | M1 | 162 | 491906 | G | A | 3 | CDZ97764 |
| X₉₁₄ | M1 | 162 | 492101 | G | A | 3 | CDZ97764 |
| X₉₁₅ | M1 | 162 | 501886 | C | T | 8 | CDZ97769 |
| X₉₁₆ | M1 | 162 | 507949 | G | A | 2 | CDZ97771 |
| X₉₁₇ | M1 | 162 | 632311 | G | A | 2 | CDZ97814 |
| X₉₁₈ | M1 | 162 | 633321 | G | A | 2 | CDZ97814 |
| X₉₁₉ | M1 | 162 | 633633 | G | A | 1 | CDZ97814 |
| X₉₂₀ | M1 | 162 | 638083 | C | T | 1 | CDZ97814 |
| X₉₂₁ | M1 | 162 | 640005 | C | T | 2 | CDZ97815 |
| X₉₂₂ | M1 | 162 | 674192 | C | T | 1 | CDZ97824 |
| X₉₂₃ | M1 | 162 | 691535 | C | T | 2 | CDZ97828 |
| X₉₂₄ | M1 | 162 | 738675 | G | A | 3 | CDZ97842 |
| X₉₂₅ | M1 | 162 | 738686 | C | T | 3 | CDZ97842 |
| X₉₂₆ | M1 | 162 | 792386 | G | A | 3 | CDZ97863 |
| X₉₂₇ | M1 | 162 | 808830 | G | A | 3 | CDZ97869 |
| X₉₂₈ | M1 | 162 | 871911 | C | T | 8 | CDZ97890 |
| X₉₂₉ | M1 | 185 | 2337 | C | T | 8 | CDZ97958 |
| X₉₃₀ | M1 | 189 | 32 | G | A | 3 | CDZ97960 |
| X₉₃₁ | M1 | 189 | 1306 | C | T | 3 | CDZ97961 |
| X₉₃₂ | M1 | 189 | 64473 | G | A | 2 | CDZ97979 |
| X₉₃₃ | M1 | 189 | 126573 | G | A | 3 | CDZ97996 |
| X₉₃₄ | M1 | 189 | 127356 | G | A | 3 | CDZ97996 |
| X₉₃₅ | M1 | 189 | 194647 | A | G | 3 | CDZ98020 |
| X₉₃₆ | M1 | 189 | 194665 | A | G | 3 | CDZ98020 |
| X₉₃₇ | M1 | 189 | 212649 | C | T | 2 | CDZ98028 |
| X₉₃₈ | M1 | 189 | 303106 | G | A | 2 | CDZ98057 |
| X₉₃₉ | M1 | 189 | 323680 | C | T | 2 | CDZ98063 |
| X₉₄₀ | M1 | 189 | 332957 | G | A | 3 | CDZ98065 |
| X₉₄₁ | M1 | 189 | 371247 | G | A | 5 | CDZ98078 |
| X₉₄₂ | M1 | 189 | 394551 | C | T | 2 | CDZ98085 |
| X₉₄₃ | M1 | 189 | 452298 | G | A | 2 | CDZ98106 |
| X₉₄₄ | M1 | 191 | 342 | A | G | 3 | CDZ98107 |
| X₉₄₅ | M1 | 191 | 347 | C | T | 3 | CDZ98107 |
| X₉₄₆ | M1 | 191 | 427 | C | T | 3 | CDZ98107 |
| X₉₄₇ | M1 | 191 | 451 | C | T | 3 | CDZ98107 |
| X₉₄₈ | M1 | 191 | 487 | A | G | 3 | CDZ98107 |
| X₉₄₉ | M1 | 197 | 624 | C | T | 3 | CDZ98111 |
| X₉₅₀ | M1 | 198 | 202 | C | A | 6 | |
| X₉₅₁ | M1 | 198 | 203 | G | A | 6 | |
| X₉₅₂ | M1 | 198 | 238 | C | T | 6 | |
| X₉₅₃ | M1 | 198 | 265 | C | T | 6 | |
| X₉₅₄ | M1 | 199 | 688 | C | T | 6 | |
| X₉₅₅ | M1 | 199 | 911 | C | T | 6 | |
| X₉₅₆ | M1 | 199 | 971 | C | T | 6 | |
| X₉₅₇ | M1 | 199 | 1218 | C | T | 6 | |
| X₉₅₈ | M1 | 202 | 171 | C | T | 6 | |
| X₉₅₉ | M1 | 205 | 360 | C | T | 3 | CDZ98112 |
| X₉₆₀ | M1 | 205 | 981 | C | T | 4 | CDZ98112 |
| X₉₆₁ | M1 | 206 | 406 | G | A | 6 | |
| X₉₆₂ | M1 | 208 | 117 | G | A | 6 | |
| X₉₆₃ | M1 | 208 | 476 | G | A | 6 | |
| X₉₆₄ | M1 | 208 | 616 | G | A | 6 | |
| X₉₆₅ | M1 | 208 | 935 | G | A | 6 | |
| X₉₆₆ | M1 | 209 | 312 | C | T | 8 | CDZ98113 |
| X₉₆₇ | M1 | 220 | 116 | C | G | 6 | |
| X₉₆₈ | M1 | 223 | 93 | G | A | 4 | CDZ98118 |
| X₉₆₉ | M1 | 223 | 102 | A | G | 4 | CDZ98118 |
| X₉₇₀ | M1 | 223 | 133 | A | G | 4 | CDZ98118 |
| X₉₇₁ | M1 | 223 | 150 | T | C | 4 | CDZ98118 |
| X₉₇₂ | M1 | 223 | 155 | A | C | 4 | CDZ98118 |
| X₉₇₃ | M1 | 223 | 158 | T | C | 4 | CDZ98118 |
| X₉₇₄ | M1 | 223 | 160 | C | T | 4 | CDZ98118 |
| X₉₇₅ | M1 | 223 | 175 | G | A | 4 | CDZ98118 |
| X₉₇₆ | M1 | 223 | 177 | A | G | 4 | CDZ98118 |
| X₉₇₇ | M1 | 224 | 27 | T | A | 6 | |
| X₉₇₈ | M1 | 225 | 317 | C | T | 6 | |
| X₉₇₉ | M1 | 227 | 202 | G | A | 6 | |
| X₉₈₀ | M1 | 232 | 340 | G | A | 3 | CDZ98119 |
| X₉₈₁ | M1 | 232 | 1379 | C | T | 1 | CDZ98119 |
| X₉₈₂ | M1 | 232 | 1521 | C | T | 2 | CDZ98119 |
| X₉₈₃ | M1 | 232 | 1662 | G | A | 2 | CDZ98119 |
| X₉₈₄ | M1 | 232 | 1761 | C | T | 2 | CDZ98119 |
| X₉₈₅ | M1 | 232 | 2027 | C | T | 4 | CDZ98119 |
| X₉₈₆ | M1 | 232 | 3060 | C | T | 2 | CDZ98120 |
| X₉₈₇ | M1 | 232 | 3284 | C | T | 4 | CDZ98119 |
| X₉₈₈ | M1 | 232 | 4437 | G | A | 2 | CDZ98121 |
| X₉₈₉ | M1 | 232 | 4561 | C | T | 3 | CDZ98120 |
| X₉₉₀ | M1 | 242 | 7334 | C | T | 2 | CDZ98125 |
| X₉₉₁ | M1 | 242 | 41710 | G | A | 3 | CDZ98131 |
| X₉₉₂ | M1 | 242 | 50262 | G | A | 8 | CDZ98135 |
| X₉₉₃ | M1 | 242 | 88037 | G | A | 2 | CDZ98147 |
| X₉₉₄ | M1 | 242 | 106805 | G | A | 3 | CDZ98152 |
| X₉₉₅ | M1 | 242 | 107587 | G | A | 4 | CDZ98153 |
| X₉₉₆ | M1 | 242 | 119446 | T | C | 1 | CDZ98156 |
| X₉₉₇ | M1 | 242 | 149113 | C | T | 2 | CDZ98165 |
| X₉₉₈ | M1 | 242 | 150461 | C | T | 3 | CDZ98164 |
| X₉₉₉ | M1 | 242 | 168689 | G | A | 1 | CDZ98172 |
| X₁₀₀₀ | M1 | 242 | 213986 | G | A | 8 | CDZ98185 |
| X₁₀₀₁ | M1 | 242 | 227271 | C | T | 3 | CDZ98191 |
| X₁₀₀₂ | M1 | 242 | 233088 | G | A | 2 | CDZ98193 |
| X₁₀₀₃ | M1 | 242 | 254710 | A | G | 9 | CDZ98200 |
| X₁₀₀₄ | M1 | 242 | 309675 | G | A | 2 | CDZ98216 |
| X₁₀₀₅ | M1 | 242 | 310353 | G | A | 2 | CDZ98216 |
| X₁₀₀₆ | M1 | 242 | 357209 | G | A | 1 | CDZ98233 |
| X₁₀₀₇ | M1 | 242 | 404132 | G | A | 3 | CDZ98248 |
| X₁₀₀₈ | M1 | 242 | 405825 | G | A | 3 | CDZ98250 |
| X₁₀₀₉ | M1 | 242 | 412676 | G | A | 3 | CDZ98249 |
| X₁₀₁₀ | M1 | 242 | 439891 | T | A | 2 | CDZ98259 |
| X₁₀₁₁ | M1 | 242 | 472562 | C | T | 3 | CDZ98266 |
| X₁₀₁₂ | M1 | 242 | 485936 | G | A | 4 | CDZ98270 |
| X₁₀₁₃ | M1 | 242 | 492389 | G | A | 2 | CDZ98274 |
| X₁₀₁₄ | M1 | 242 | 514780 | C | T | 2 | CDZ98282 |
| X₁₀₁₅ | M1 | 242 | 542663 | G | A | 3 | CDZ98290 |
| X₁₀₁₆ | M1 | 242 | 545031 | G | A | 3 | CDZ98293 |
| X₁₀₁₇ | M1 | 242 | 559999 | G | A | 2 | CDZ98297 |
| X₁₀₁₈ | M1 | 242 | 577367 | G | A | 3 | CDZ98302 |
| X₁₀₁₉ | M1 | 242 | 602456 | G | A | 2 | CDZ98308 |
| X₁₀₂₀ | M1 | 242 | 602531 | G | A | 2 | CDZ98308 |
| X₁₀₂₁ | M1 | 242 | 604704 | C | T | 2 | CDZ98309 |
| X₁₀₂₂ | M1 | 249 | 206 | G | A | 4 | CED84929 |
| X₁₀₂₃ | M1 | 249 | 7968 | A | T | 3 | CED84930 |
| X₁₀₂₄ | M1 | 249 | 42295 | C | T | 2 | CED84942 |
| X₁₀₂₅ | M1 | 249 | 72016 | C | T | 3 | CED84950 |
| X₁₀₂₆ | M1 | 249 | 142920 | G | A | 1 | CED84978 |
| X₁₀₂₇ | M1 | 249 | 157550 | C | T | 8 | CED84983 |
| X₁₀₂₈ | M1 | 249 | 159819 | C | T | 1 | CED84984 |
| X₁₀₂₉ | M1 | 249 | 191304 | C | T | 2 | CED84988 |
| X₁₀₃₀ | M1 | 249 | 269707 | G | A | 2 | CED85013 |
| X₁₀₃₁ | M1 | 249 | 272075 | C | T | 8 | CED85013 |
| X₁₀₃₂ | M1 | 249 | 289154 | C | T | 2 | CED85018 |
| X₁₀₃₃ | M1 | 249 | 305975 | G | A | 2 | CED85023 |
| X₁₀₃₄ | M1 | 249 | 342990 | G | A | 2 | CED85036 |
| X₁₀₃₅ | M1 | 249 | 371535 | G | A | 2 | CED85048 |
| X₁₀₃₆ | M1 | 249 | 377814 | G | A | 3 | CED85051 |
| X₁₀₃₇ | M1 | 249 | 485782 | C | T | 3 | CED85089 |
| X₁₀₃₈ | M1 | 249 | 487588 | G | A | 2 | CED85090 |
| X₁₀₃₉ | M1 | 249 | 500195 | C | T | 8 | CED85093 |
| X₁₀₄₀ | M1 | 249 | 501310 | C | T | 3 | CED85093 |
| X₁₀₄₁ | M1 | 249 | 501510 | T | C | 3 | CED85093 |
| X₁₀₄₂ | M1 | 249 | 509901 | C | T | 1 | CED85096 |
| X₁₀₄₃ | M1 | 249 | 532943 | A | G | 3 | CED85099 |
| X₁₀₄₄ | M1 | 249 | 565916 | C | T | 4 | CED85113 |
| X₁₀₄₅ | M1 | 249 | 568518 | C | T | 2 | CED85115 |
| X₁₀₄₆ | M1 | 249 | 574798 | C | T | 1 | CED85117 |
| X₁₀₄₇ | M1 | 249 | 598579 | C | T | 2 | CED85125 |
| X₁₀₄₈ | M1 | 249 | 637944 | G | A | 1 | CED85135 |
| X₁₀₄₉ | M1 | 249 | 654772 | C | T | 4 | CED85141 |
| X₁₀₅₀ | M1 | 249 | 660408 | G | A | 2 | CED85144 |
| X₁₀₅₁ | M1 | 249 | 715551 | G | A | 3 | CED85163 |
| X₁₀₅₂ | M1 | 249 | 772481 | G | A | 3 | CED85180 |
| X₁₀₅₃ | M1 | 249 | 854220 | G | A | 2 | CED85201 |
| X₁₀₅₄ | M1 | 249 | 871689 | G | A | 5 | CED85204 |
| X₁₀₅₅ | M1 | 249 | 889853 | G | A | 1 | CED85211 |
| X₁₀₅₆ | M1 | 249 | 911274 | C | T | 2 | CED85218 |
| X₁₀₅₇ | M1 | 249 | 927876 | C | T | 4 | CED85222 |
| X₁₀₅₈ | M1 | 249 | 932606 | C | T | 3 | CED85223 |
| X₁₀₅₉ | M1 | 249 | 945434 | C | T | 3 | CED85227 |
| X₁₀₆₀ | M1 | 249 | 945727 | C | T | 3 | CED85227 |
| X₁₀₆₁ | M1 | 249 | 969333 | G | T | 3 | CED85238 |
| X₁₀₆₂ | M1 | 249 | 977170 | C | T | 2 | CED85242 |
| X₁₀₆₃ | M1 | 249 | 989520 | C | T | 3 | CED85243 |
| X₁₀₆₄ | M1 | 249 | 1014098 | C | T | 2 | CED85252 |
| X₁₀₆₅ | M1 | 249 | 1014926 | C | T | 2 | CED85252 |
| X₁₀₆₆ | M1 | 249 | 1021827 | C | T | 2 | CED85254 |
| X₁₀₆₇ | M1 | 249 | 1050123 | C | T | 2 | CED85262 |
| X₁₀₆₈ | M1 | 249 | 1076166 | C | T | 1 | CED85270 |
| X₁₀₆₉ | M1 | 249 | 1114790 | G | A | 1 | CED85283 |
| X₁₀₇₀ | M1 | 249 | 1127638 | C | T | 2 | CED85286 |
| X₁₀₇₁ | M1 | 249 | 1144309 | G | A | 3 | CED85291 |
| X₁₀₇₂ | M1 | 249 | 1155955 | C | T | 3 | CED85297 |
| X₁₀₇₃ | M1 | 249 | 1176977 | G | A | 1 | CED85303 |
| X₁₀₇₄ | M1 | 249 | 1195367 | G | A | 3 | CED85307 |
| X₁₀₇₅ | M1 | 249 | 1213338 | C | T | 3 | CED85314 |
| X₁₀₇₆ | M1 | 249 | 1230068 | C | T | 2 | CED85319 |
| X₁₀₇₇ | M1 | 249 | 1286733 | G | A | 3 | CED85336 |
| X₁₀₇₈ | M1 | 249 | 1299472 | C | T | 1 | CED85343 |
| X₁₀₇₉ | M1 | 249 | 1331428 | G | A | 3 | CED85352 |
| X₁₀₈₀ | M1 | 249 | 1350079 | G | A | 2 | CED85357 |
| X₁₀₈₁ | M1 | 249 | 1359399 | G | A | 1 | CED85360 |
| X₁₀₈₂ | M1 | 249 | 1391183 | C | T | 2 | CED85372 |
| X₁₀₈₃ | M1 | 249 | 1452855 | C | T | 2 | CED85396 |
| X₁₀₈₄ | M1 | 249 | 1452994 | C | T | 3 | CED85394 |
| X₁₀₈₅ | M1 | 249 | 1471661 | C | T | 1 | CED85402 |
| X₁₀₈₆ | M1 | 249 | 1486828 | G | A | 1 | CED85411 |
| X₁₀₈₇ | M1 | 249 | 1486831 | T | C | 1 | CED85411 |
| X₁₀₈₈ | M1 | 249 | 1498029 | T | C | 3 | CED85415 |
| X₁₀₈₉ | M1 | 249 | 1498823 | T | C | 2 | CED85414 |
| X₁₀₉₀ | M1 | 249 | 1498829 | C | T | 2 | CED85414 |
| X₁₀₉₁ | M1 | 249 | 1499741 | G | A | 2 | CED85415 |
| X₁₀₉₂ | M1 | 249 | 1502631 | G | T | 2 | CED85416 |
| X₁₀₉₃ | M1 | 249 | 1502641 | T | A | 2 | CED85416 |
| X₁₀₉₄ | M1 | 249 | 1502819 | T | G | 1 | CED85416 |
| X₁₀₉₅ | M1 | 249 | 1505138 | G | A | 3 | CED85416 |
| X₁₀₉₆ | M1 | 249 | 1518591 | T | A | 3 | CED85419 |
| X₁₀₉₇ | M1 | 249 | 1519431 | A | G | 3 | CED85419 |
| X₁₀₉₈ | M1 | 249 | 1520169 | C | T | 1 | CED85420 |
| X₁₀₉₉ | M1 | 249 | 1567124 | G | A | 3 | CED85432 |
| X₁₁₀₀ | M1 | 249 | 1599907 | C | T | 3 | CED85444 |
| X₁₁₀₁ | M1 | 249 | 1619036 | G | A | 2 | CED85450 |
| X₁₁₀₂ | M1 | 249 | 1653615 | T | G | 2 | CED85457 |
| X₁₁₀₃ | M1 | 249 | 1653962 | C | T | 4 | CED85458 |
| X₁₁₀₄ | M1 | 249 | 1666185 | C | T | 3 | CED85460 |
| X₁₁₀₅ | M1 | 249 | 1726421 | G | A | 2 | CED85476 |
| X₁₁₀₆ | M1 | 249 | 1726777 | C | T | 3 | CED85474 |
| X₁₁₀₇ | M1 | 249 | 1729264 | C | T | 1 | CED85476 |
| X₁₁₀₈ | M1 | 249 | 1741408 | C | T | 1 | CED85480 |
| X₁₁₀₉ | M1 | 249 | 1760715 | C | T | 3 | CED85489 |
| X₁₁₁₀ | M1 | 249 | 1780551 | A | G | 3 | CED85493 |
| X₁₁₁₁ | M1 | 249 | 1780569 | A | G | 3 | CED85493 |
| X₁₁₁₂ | M1 | 249 | 1786050 | C | T | 3 | CED85496 |
| X₁₁₁₃ | M1 | 249 | 1801965 | G | A | 3 | CED85501 |
| X₁₁₁₄ | M1 | 249 | 1803575 | C | T | 2 | CED85502 |
| X₁₁₁₅ | M1 | 249 | 1804835 | G | A | 2 | CED85502 |
| X₁₁₁₆ | M1 | 249 | 1834426 | G | A | 2 | CED85511 |
| X₁₁₁₇ | M1 | 249 | 1862072 | C | T | 1 | CED85520 |
| X₁₁₁₈ | M1 | 249 | 1880762 | G | A | 1 | CED85525 |
| X₁₁₁₉ | M1 | 249 | 1944026 | G | A | 1 | CED85545 |
| X₁₁₂₀ | M1 | 249 | 1963639 | G | T | 2 | CED85550 |
| X₁₁₂₁ | M1 | 249 | 1981230 | G | A | 3 | CED85557 |
| X₁₁₂₂ | M1 | 249 | 1988505 | C | T | 3 | CED85560 |
| X₁₁₂₃ | M1 | 249 | 2010712 | G | A | 3 | CED85564 |
| X₁₁₂₄ | M1 | 249 | 2015272 | G | A | 2 | CED85567 |
| X₁₁₂₅ | M1 | 249 | 2027477 | G | A | 1 | CED85573 |
| X₁₁₂₆ | M1 | 249 | 2032298 | C | T | 2 | CED85575 |
| X₁₁₂₇ | M1 | 249 | 2063221 | G | A | 2 | CED85586 |
| X₁₁₂₈ | M1 | 249 | 2080239 | G | A | 3 | CED85590 |
| X₁₁₂₉ | M1 | 249 | 2110956 | C | T | 1 | CED85603 |
| X₁₁₃₀ | M1 | 249 | 2124517 | C | T | 2 | CED85607 |
| X₁₁₃₁ | M1 | 249 | 2183695 | G | A | 2 | CED85623 |
| X₁₁₃₂ | M1 | 249 | 2198205 | G | A | 8 | CED85628 |
| X₁₁₃₃ | M1 | 249 | 2223626 | G | A | 1 | CED85633 |
| X₁₁₃₄ | M1 | 249 | 2255547 | G | C | 3 | CED85640 |
| X₁₁₃₅ | M1 | 251 | 874 | G | A | 6 | |
| X₁₁₃₆ | M1 | 252 | 512 | C | T | 6 | |
| X₁₁₃₇ | M1 | 252 | 524 | C | T | 6 | |
| X₁₁₃₈ | M1 | 258 | 267 | G | A | 6 | |
| X₁₁₃₉ | M1 | 258 | 1139 | A | T | 6 | |
| X₁₁₄₀ | M1 | 259 | 91 | G | C | 6 | |
| X₁₁₄₁ | M1 | 260 | 156 | G | A | 6 | |
| X₁₁₄₂ | M1 | 262 | 4049 | G | A | 1 | CDZ98330 |
| X₁₁₄₃ | M1 | 262 | 20959 | G | A | 1 | CDZ98336 |
| X₁₁₄₄ | M1 | 262 | 30347 | G | A | 2 | CDZ98338 |
| X₁₁₄₅ | M1 | 262 | 30359 | G | A | 2 | CDZ98338 |
| X₁₁₄₆ | M1 | 262 | 63941 | G | A | 3 | CDZ98349 |
| X₁₁₄₇ | M1 | 262 | 81226 | G | A | 2 | CDZ98358 |
| X₁₁₄₈ | M1 | 262 | 96076 | C | T | 8 | CDZ98364 |
| X₁₁₄₉ | M1 | 262 | 130174 | C | T | 3 | CDZ98372 |
| X₁₁₅₀ | M1 | 262 | 148151 | C | T | 3 | CDZ98375 |
| X₁₁₅₁ | M1 | 262 | 148199 | G | A | 2 | CDZ98376 |
| X₁₁₅₂ | M1 | 262 | 164438 | G | A | 2 | CDZ98383 |
| X₁₁₅₃ | M1 | 262 | 191816 | C | T | 2 | CDZ98393 |
| X₁₁₅₄ | M1 | 262 | 201531 | G | A | 8 | CDZ98396 |
| X₁₁₅₅ | M1 | 262 | 221937 | C | T | 3 | CDZ98403 |
| X₁₁₅₆ | M1 | 262 | 225924 | G | A | 3 | CDZ98404 |
| X₁₁₅₇ | M1 | 262 | 242371 | C | T | 3 | CDZ98411 |
| X₁₁₅₈ | M1 | 262 | 250756 | A | G | 3 | CDZ98412 |
| X₁₁₅₉ | M1 | 262 | 319302 | G | A | 1 | CDZ98436 |
| X₁₁₆₀ | M1 | 262 | 350852 | C | T | 8 | CDZ98449 |
| X₁₁₆₁ | M1 | 262 | 359947 | A | G | 1 | CDZ98451 |
| X₁₁₆₂ | M1 | 262 | 366171 | G | A | 3 | CDZ98452 |
| X₁₁₆₃ | M1 | 262 | 380519 | C | T | 4 | CDZ98457 |
| X₁₁₆₄ | M1 | 262 | 383215 | C | T | 8 | CDZ98459 |
| X₁₁₆₅ | M1 | 262 | 390030 | C | T | 2 | CDZ98460 |
| X₁₁₆₆ | M1 | 262 | 397539 | G | A | 3 | CDZ98463 |
| X₁₁₆₇ | M1 | 262 | 420102 | G | A | 2 | CDZ98468 |
| X₁₁₆₈ | M1 | 262 | 444331 | G | A | 2 | CDZ98477 |
| X₁₁₆₉ | M1 | 262 | 446956 | C | T | 2 | CDZ98477 |
| X₁₁₇₀ | M1 | 262 | 451669 | C | T | 3 | CDZ98478 |
| X₁₁₇₁ | M1 | 262 | 498239 | C | T | 8 | CDZ98496 |
| X₁₁₇₂ | M1 | 262 | 503840 | G | A | 2 | CDZ98499 |
| X₁₁₇₃ | M1 | 262 | 525946 | C | T | 2 | CDZ98509 |
| X₁₁₇₄ | M1 | 262 | 539452 | A | G | 3 | CDZ98514 |
| X₁₁₇₅ | M1 | 262 | 539470 | A | G | 3 | CDZ98514 |
| X₁₁₇₆ | M1 | 262 | 552414 | C | T | 1 | CDZ98518 |
| X₁₁₇₇ | M1 | 262 | 557280 | G | A | 2 | CDZ98518 |
| X₁₁₇₈ | M1 | 262 | 560713 | G | A | 8 | CDZ98519 |
| X₁₁₇₉ | M1 | 262 | 562795 | C | T | 2 | CDZ98521 |
| X₁₁₈₀ | M1 | 262 | 567591 | G | A | 2 | CDZ98524 |
| X₁₁₈₁ | M1 | 262 | 567801 | G | A | 2 | CDZ98524 |
| X₁₁₈₂ | M1 | 262 | 573667 | C | T | 2 | CDZ98526 |
| X₁₁₈₃ | M1 | 262 | 618198 | G | A | 3 | CDZ98538 |
| X₁₁₈₄ | M1 | 262 | 627705 | C | T | 3 | CDZ98542 |
| X₁₁₈₅ | M1 | 262 | 638804 | G | C | 3 | CDZ98544 |
| X₁₁₈₆ | M1 | 262 | 639962 | A | G | 4 | CDZ98544 |
| X₁₁₈₇ | M1 | 262 | 640066 | A | G | 4 | CDZ98544 |
| X₁₁₈₈ | M1 | 262 | 653920 | A | G | 4 | CDZ98545 |
| X₁₁₈₉ | M1 | 262 | 656031 | C | A | 4 | CDZ98545 |
| X₁₁₉₀ | M1 | 262 | 668764 | C | T | 3 | CDZ98545 |
| X₁₁₉₁ | M1 | 262 | 670988 | C | T | 2 | CDZ98547 |
| X₁₁₉₂ | M1 | 262 | 691577 | C | T | 2 | CDZ98551 |
| X₁₁₉₃ | M1 | 262 | 707203 | C | T | 2 | CDZ98556 |
| X₁₁₉₄ | M1 | 262 | 751209 | C | T | 2 | CDZ98572 |
| X₁₁₉₅ | M1 | 262 | 771330 | G | A | 2 | CDZ98578 |
| X₁₁₉₆ | M1 | 262 | 822734 | G | A | 2 | CDZ98597 |
| X₁₁₉₇ | M1 | 262 | 831789 | C | T | 1 | CDZ98601 |
| X₁₁₉₈ | M1 | 262 | 835874 | C | T | 3 | CDZ98601 |
| X₁₁₉₉ | M1 | 262 | 856616 | G | A | 8 | CDZ98612 |
| X₁₂₀₀ | M1 | 262 | 865999 | G | A | 3 | CDZ98613 |
| X₁₂₀₁ | M1 | 262 | 971713 | G | A | 3 | CDZ98649 |
| X₁₂₀₂ | M1 | 262 | 1076581 | G | A | 3 | CDZ98679 |
| X₁₂₀₃ | M1 | 262 | 1140198 | C | T | 2 | CDZ98701 |
| X₁₂₀₄ | M1 | 262 | 1209662 | G | A | 2 | CDZ98723 |
| X₁₂₀₅ | M1 | 262 | 1225705 | C | T | 2 | CDZ98730 |
| X₁₂₀₆ | M1 | 262 | 1226072 | C | T | 2 | CDZ98730 |
| X₁₂₀₇ | M1 | 262 | 1229407 | G | A | 3 | CDZ98732 |
| X₁₂₀₈ | M1 | 262 | 1233054 | G | A | 1 | CDZ98733 |
| X₁₂₀₉ | M1 | 262 | 1262331 | C | T | 3 | CDZ98740 |
| X₁₂₁₀ | M1 | 262 | 1277604 | G | A | 1 | CDZ98747 |
| X₁₂₁₁ | M1 | 262 | 1284428 | G | A | 1 | CDZ98748 |
| X₁₂₁₂ | M1 | 262 | 1305361 | G | A | 3 | CDZ98753 |
| X₁₂₁₃ | M1 | 262 | 1329643 | C | T | 3 | CDZ98760 |
| X₁₂₁₄ | M1 | 262 | 1332176 | C | T | 2 | CDZ98761 |
| X₁₂₁₅ | M1 | 262 | 1349561 | G | A | 2 | CDZ98766 |
| X₁₂₁₆ | M1 | 262 | 1351116 | A | G | 1 | CDZ98766 |
| X₁₂₁₇ | M1 | 262 | 1367361 | C | T | 2 | CDZ98772 |
| X₁₂₁₈ | M1 | 262 | 1408928 | G | A | 3 | CDZ98786 |
| X₁₂₁₉ | M1 | 262 | 1424293 | G | A | 2 | CDZ98791 |
| X₁₂₂₀ | M1 | 26 | 1455677 | G | A | 5 | CDZ98804 |
| X₁₂₂₁ | M1 | 262 | 1546246 | C | T | 3 | CDZ98833 |
| X₁₂₂₂ | M1 | 262 | 1549845 | C | T | 2 | CDZ98837 |
| X₁₂₂₃ | M1 | 262 | 1550311 | C | T | 3 | CDZ98836 |
| X₁₂₂₄ | M1 | 262 | 1550456 | C | T | 1 | CDZ98837 |
| X₁₂₂₅ | M1 | 262 | 1615052 | G | T | 2 | CDZ98855 |
| X₁₂₂₆ | M1 | 262 | 1615762 | G | A | 2 | CDZ98855 |
| X₁₂₂₇ | M1 | 262 | 1684375 | G | A | 2 | CDZ98876 |
| X₁₂₂₈ | M1 | 262 | 1692423 | T | C | 3 | CDZ98877 |
| X₁₂₂₉ | M1 | 262 | 1694625 | C | T | 3 | CDZ98878 |
| X₁₂₃₀ | M1 | 262 | 1695377 | C | T | 3 | CDZ98878 |
| X₁₂₃₁ | M1 | 262 | 1709112 | G | A | 3 | CDZ98884 |
| X₁₂₃₂ | M1 | 262 | 1711527 | G | A | 1 | CDZ98886 |
| X₁₂₃₃ | M1 | 262 | 1712334 | G | A | 2 | CDZ98886 |
| X₁₂₃₄ | M1 | 262 | 1713238 | G | A | 4 | CDZ98885 |
| X₁₂₃₅ | M1 | 262 | 1716385 | G | A | 3 | CDZ98887 |
| X₁₂₃₆ | M1 | 266 | 85 | C | T | 6 | |
| X₁₂₃₇ | M1 | 266 | 252 | C | T | 6 | |
| X₁₂₃₈ | M2 | 22 | 1643 | C | T | 2 | CED80056 |
| X₁₂₃₉ | M2 | 22 | 2524 | G | A | 4 | CED80056 |
| X₁₂₄₀ | M2 | 28 | 236 | G | A | 3 | CDZ96150 |
| X₁₂₄₁ | M2 | 28 | 377 | G | A | 3 | CDZ96150 |
| X₁₂₄₂ | M2 | 28 | 1506 | C | T | 2 | CDZ96150 |
| X₁₂₄₃ | M2 | 28 | 1677 | C | T | 4 | CDZ96150 |
| X₁₂₄₄ | M2 | 43 | 464 | G | A | 2 | CDZ96151 |
| X₁₂₄₅ | M2 | 43 | 753 | C | T | 2 | CDZ96151 |
| X₁₂₄₆ | M2 | 43 | 757 | G | A | 1 | CDZ96151 |
| X₁₂₄₇ | M2 | 43 | 1527 | C | T | 3 | CDZ96151 |
| X₁₂₄₈ | M2 | 43 | 1748 | G | A | 3 | CDZ96151 |
| X₁₂₄₉ | M2 | 43 | 1928 | C | T | 3 | CDZ96151 |
| X₁₂₅₀ | M2 | 62 | 26 | T | A | 4 | CDZ96152 |
| X₁₂₅₁ | M2 | 62 | 27 | T | C | 4 | CDZ96152 |
| X₁₂₅₂ | M2 | 62 | 55 | T | A | 4 | CDZ96152 |
| X₁₂₅₃ | M2 | 62 | 403 | C | T | 1 | CDZ96152 |
| X₁₂₅₄ | M2 | 62 | 875 | G | A | 2 | CDZ96152 |
| X₁₂₅₅ | M2 | 102 | 339 | C | T | 4 | CDZ96153 |
| X₁₂₅₆ | M2 | 102 | 951 | G | A | 4 | CDZ96153 |
| X₁₂₅₇ | M2 | 102 | 1042 | G | A | 4 | CDZ96153 |
| X₁₂₅₈ | M2 | 102 | 2334 | G | A | 2 | CDZ96153 |
| X₁₂₅₉ | M2 | 102 | 2590 | C | T | 2 | CDZ96153 |
| X₁₂₆₀ | M2 | 102 | 3427 | C | T | 3 | CDZ96153 |
| X₁₂₆₁ | M2 | 176 | 358 | G | A | 3 | CED80058 |
| X₁₂₆₂ | M2 | 176 | 1872 | G | A | 4 | CED80058 |
| X₁₂₆₃ | M2 | 176 | 1980 | G | A | 4 | CED80058 |
| X₁₂₆₄ | M2 | 176 | 2335 | A | G | 4 | CED80058 |
| X₁₂₆₅ | M2 | 176 | 2349 | G | A | 4 | CED80058 |
| X₁₂₆₆ | M2 | 179 | 1525 | G | A | 7 | CDZ96154 |
| X₁₂₆₇ | M2 | 179 | 1780 | C | T | 7 | CDZ96154 |
| X₁₂₆₈ | M2 | 188 | 815 | C | T | 2 | CED80060 |
| X₁₂₆₉ | M2 | 188 | 1174 | C | T | 4 | CED80060 |
| X₁₂₇₀ | M2 | 188 | 2163 | G | A | 4 | CED80060 |
| X₁₂₇₁ | M2 | 188 | 2170 | G | A | 4 | CED80060 |
| X₁₂₇₂ | M2 | 247 | 102 | G | A | 4 | CED80061 |
| X₁₂₇₃ | M2 | 247 | 989 | C | T | 2 | CED80061 |
| X₁₂₇₄ | M2 | 247 | 1053 | G | A | 2 | CED80061 |
| X₁₂₇₅ | M2 | 3 | 142 | C | T | 6 | |
| X₁₂₇₆ | M2 | 7 | 160 | T | C | 6 | |
| X₁₂₇₇ | M2 | 8 | 1110 | C | T | 6 | |
| X₁₂₇₈ | M2 | 11 | 154 | A | C | 6 | |
| X₁₂₇₉ | M2 | 11 | 158 | C | T | 6 | |
| X₁₂₈₀ | M2 | 15 | 28 | C | G | 6 | |
| X₁₂₈₁ | M2 | 24 | 513 | T | C | 3 | CDZ96160 |
| X₁₂₈₂ | M2 | 24 | 520 | C | G | 3 | CDZ96160 |
| X₁₂₈₃ | M2 | 24 | 5850 | C | T | 2 | CDZ96160 |
| X₁₂₈₄ | M2 | 24 | 85162 | G | A | 2 | CDZ96190 |
| X₁₂₈₅ | M2 | 24 | 169574 | C | T | 3 | CDZ96213 |
| X₁₂₈₆ | M2 | 24 | 180340 | G | A | 3 | CDZ96216 |
| X₁₂₈₇ | M2 | 24 | 197593 | C | T | 2 | CDZ96224 |
| X₁₂₈₈ | M2 | 24 | 236940 | G | A | 3 | CDZ96232 |
| X₁₂₈₉ | M2 | 24 | 266697 | G | A | 1 | CDZ96242 |
| X₁₂₉₀ | M2 | 24 | 278319 | G | A | 3 | CDZ96245 |
| X₁₂₉₁ | M2 | 24 | 307114 | G | A | 2 | CDZ96255 |
| X₁₂₉₂ | M2 | 24 | 340663 | T | C | 3 | CDZ96265 |
| X₁₂₉₃ | M2 | 24 | 363770 | C | T | 2 | CDZ96275 |
| X₁₂₉₄ | M2 | 24 | 420108 | G | A | 3 | CDZ96291 |
| X₁₂₉₅ | M2 | 24 | 470687 | G | A | 3 | CDZ96308 |
| X₁₂₉₆ | M2 | 24 | 473232 | A | G | 2 | CDZ96308 |
| X₁₂₉₇ | M2 | 24 | 491396 | C | T | 3 | CDZ96313 |
| X₁₂₉₈ | M2 | 27 | 445 | C | T | 6 | |
| X₁₂₉₉ | M2 | 27 | 1012 | A | C | 6 | |
| X₁₃₀₀ | M2 | 27 | 1013 | T | A | 6 | |
| X₁₃₀₁ | M2 | 32 | 158 | C | T | 3 | CDZ96332 |
| X₁₃₀₂ | M2 | 32 | 215 | C | T | 3 | CDZ96332 |
| X₁₃₀₃ | M2 | 32 | 2438 | C | T | 3 | CDZ96332 |
| X₁₃₀₄ | M2 | 32 | 2734 | C | T | 1 | CDZ96332 |
| X₁₃₀₅ | M2 | 32 | 2821 | C | T | 7 | CDZ96332 |
| X₁₃₀₆ | M2 | 32 | 3067 | C | T | 2 | CDZ96332 |
| X₁₃₀₇ | M2 | 32 | 3506 | C | T | 4 | CDZ96332 |
| X₁₃₀₈ | M2 | 32 | 3562 | G | A | 4 | CDZ96332 |
| X₁₃₀₉ | M2 | 33 | 399 | T | A | 4 | CED82002 |
| X₁₃₁₀ | M2 | 33 | 24784 | C | T | 2 | CED82008 |
| X₁₃₁₁ | M2 | 33 | 45488 | C | T | 3 | CED82012 |
| X₁₃₁₂ | M2 | 33 | 79913 | G | A | 1 | CED82024 |
| X₁₃₁₃ | M2 | 33 | 109397 | G | A | 2 | CED82038 |
| X₁₃₁₄ | M2 | 33 | 109494 | G | A | 2 | CED82038 |
| X₁₃₁₅ | M2 | 33 | 182417 | G | A | 3 | CED82062 |
| X₁₃₁₆ | M2 | 33 | 192563 | C | T | 2 | CED82066 |
| X₁₃₁₇ | M2 | 33 | 257786 | C | T | 1 | CED82084 |
| X₁₃₁₈ | M2 | 33 | 260027 | G | A | 3 | CED82084 |
| X₁₃₁₉ | M2 | 33 | 274627 | G | A | 8 | CED82091 |
| X₁₃₂₀ | M2 | 33 | 283808 | C | T | 2 | CED82093 |
| X₁₃₂₁ | M2 | 33 | 311532 | G | A | 3 | CED82101 |
| X₁₃₂₂ | M2 | 33 | 318242 | G | A | 3 | CED82105 |
| X₁₃₂₃ | M2 | 33 | 325745 | C | T | 2 | CED82108 |
| X₁₃₂₄ | M2 | 33 | 327840 | C | T | 1 | CED82108 |
| X₁₃₂₅ | M2 | 33 | 341890 | G | A | 2 | CED82113 |
| X₁₃₂₆ | M2 | 33 | 374131 | A | G | 6 | CED82121- |
| | | | | | | | CED82122 |
| X₁₃₂₇ | M2 | 33 | 374134 | A | G | 6 | CED82121- |
| | | | | | | | CED82122 |
| X₁₃₂₈ | M2 | 33 | 374137 | A | T | 6 | CED82121- |
| | | | | | | | CED82122 |
| X₁₃₂₉ | M2 | 33 | 374141 | C | T | 6 | CED82121- |
| | | | | | | | CED82122 |
| X₁₃₃₀ | M2 | 33 | 374150 | A | C | 6 | CED82121- |
| | | | | | | | CED82122 |
| X₁₃₃₁ | M2 | 33 | 375339 | A | G | 6 | CED82121- |
| | | | | | | | CED82122 |
| X₁₃₃₂ | M2 | 33 | 375366 | T | C | 6 | CED82121- |
| | | | | | | | CED82122 |
| X₁₃₃₃ | M2 | 33 | 382804 | G | A | 3 | CED82122 |
| X₁₃₃₄ | M2 | 33 | 385139 | G | C | 2 | CED82122 |
| X₁₃₃₅ | M2 | 33 | 385319 | A | C | 2 | CED82122 |
| X₁₃₃₆ | M2 | 33 | 386225 | G | A | 3 | CED82124 |
| X₁₃₃₇ | M2 | 33 | 397625 | C | T | 3 | CED82125 |
| X₁₃₃₈ | M2 | 33 | 448815 | G | A | 2 | CED82144 |
| X₁₃₃₉ | M2 | 33 | 453975 | C | T | 2 | CED82147 |
| X₁₃₄₀ | M2 | 33 | 455451 | C | T | 3 | CED82146 |
| X₁₃₄₁ | M2 | 33 | 509127 | C | T | 3 | CED82166 |
| X₁₃₄₂ | M2 | 33 | 543070 | C | T | 3 | CED82179 |
| X₁₃₄₃ | M2 | 33 | 606302 | G | T | 5 | CED82200 |
| X₁₃₄₄ | M2 | 33 | 643436 | G | A | 3 | CED82208 |
| X₁₃₄₅ | M2 | 33 | 652012 | G | A | 2 | CED82212 |
| X₁₃₄₆ | M2 | 33 | 690180 | C | T | 1 | CED82224 |
| X₁₃₄₇ | M2 | 33 | 693516 | C | T | 2 | CED82225 |
| X₁₃₄₈ | M2 | 33 | 699423 | C | T | 1 | CED82227 |
| X₁₃₄₉ | M2 | 33 | 774105 | G | A | 2 | CED82248 |
| X₁₃₅₀ | M2 | 33 | 790955 | C | T | 3 | CED82253 |
| X₁₃₅₁ | M2 | 33 | 812031 | G | T | 2 | CED82262 |
| X₁₃₅₂ | M2 | 33 | 816048 | C | T | 3 | CED82262 |
| X₁₃₅₃ | M2 | 33 | 822243 | C | T | 2 | CED82267 |
| X₁₃₅₄ | M2 | 33 | 850373 | G | A | 1 | CED82278 |
| X₁₃₅₅ | M2 | 33 | 929585 | T | C | 1 | CED82297 |
| X₁₃₅₆ | M2 | 33 | 936350 | T | A | 3 | CED82300 |
| X₁₃₅₇ | M2 | 33 | 936356 | G | A | 3 | CED82300 |
| X₁₃₅₈ | M2 | 33 | 936571 | A | G | 3 | CED82300 |
| X₁₃₅₉ | M2 | 33 | 936600 | G | A | 3 | CED82300 |
| X₁₃₆₀ | M2 | 33 | 982324 | C | T | 4 | CED82312 |
| X₁₃₆₁ | M2 | 33 | 983105 | G | A | 3 | CED82315 |
| X₁₃₆₂ | M2 | 33 | 995886 | G | A | 1 | CED82318 |
| X₁₃₆₃ | M2 | 33 | 1049699 | G | A | 3 | CED82334 |
| X₁₃₆₄ | M2 | 33 | 1061503 | C | A | 2 | CED82340 |
| X₁₃₆₅ | M2 | 33 | 1086609 | G | A | 1 | CED82349 |
| X₁₃₆₆ | M2 | 33 | 1087773 | G | A | 2 | CED82349 |
| X₁₃₆₇ | M2 | 33 | 1087834 | G | A | 3 | CED82350 |
| X₁₃₆₈ | M2 | 33 | 1122362 | C | T | 2 | CED82358 |
| X₁₃₆₉ | M2 | 33 | 1126814 | G | A | 2 | CED82358 |
| X₁₃₇₀ | M2 | 33 | 1137501 | G | A | 2 | CED82362 |
| X₁₃₇₁ | M2 | 33 | 1138444 | C | T | 3 | CED82363 |
| X₁₃₇₂ | M2 | 33 | 1151553 | C | T | 2 | CED82367 |
| X₁₃₇₃ | M2 | 33 | 1165914 | G | A | 2 | CED82372 |
| X₁₃₇₄ | M2 | 33 | 1191727 | C | T | 2 | CED82379 |
| X₁₃₇₅ | M2 | 33 | 1277762 | G | A | 1 | CED82402 |
| X₁₃₇₆ | M2 | 33 | 1311181 | G | A | 3 | CED82413 |
| X₁₃₇₇ | M2 | 33 | 1318841 | C | T | 3 | CED82414 |
| X₁₃₇₈ | M2 | 33 | 1324395 | G | A | 3 | CED82417 |
| X₁₃₇₉ | M2 | 33 | 1333932 | G | A | 3 | CED82421 |
| X₁₃₈₀ | M2 | 33 | 1339376 | C | T | 2 | CED82424 |
| X₁₃₈₁ | M2 | 33 | 1399499 | C | T | 3 | CED82444 |
| X₁₃₈₂ | M2 | 33 | 1399524 | C | T | 3 | CED82444 |
| X₁₃₈₃ | M2 | 33 | 1468155 | G | A | 2 | CED82464 |
| X₁₃₈₄ | M2 | 33 | 1480984 | G | A | 3 | CED82466 |
| X₁₃₈₅ | M2 | 33 | 1499347 | G | A | 3 | CED82472 |
| X₁₃₈₆ | M2 | 33 | 1502644 | G | A | 2 | CED82474 |
| X₁₃₈₇ | M2 | 33 | 1515369 | G | A | 3 | CED82478 |
| X₁₃₈₈ | M2 | 33 | 1544767 | A | C | 6 | CED82485- |
| | | | | | | | CED82486 |
| X₁₃₈₉ | M2 | 33 | 1544951 | C | G | 6 | CED82485- |
| | | | | | | | CED82486 |
| X₁₃₉₀ | M2 | 33 | 1572498 | C | T | 8 | CED82491 |
| X₁₃₉₁ | M2 | 33 | 1587443 | C | T | 3 | CED82497 |
| X₁₃₉₂ | M2 | 33 | 1596242 | C | T | 3 | CED82498 |
| X₁₃₉₃ | M2 | 33 | 1638726 | G | A | 3 | CED82513 |
| X₁₃₉₄ | M2 | 33 | 1642659 | G | A | 2 | CED82515 |
| X₁₃₉₅ | M2 | 33 | 1666039 | G | A | 3 | CED82521 |
| X₁₃₉₆ | M2 | 33 | 1698181 | G | A | 3 | CED82531 |
| X₁₃₉₇ | M2 | 33 | 1700000 | G | A | 2 | CED82534 |
| X₁₃₉₈ | M2 | 33 | 1714108 | G | A | 1 | CED82537 |
| X₁₃₉₉ | M2 | 33 | 1714997 | G | A | 2 | CED82538 |
| X₁₄₀₀ | M2 | 33 | 1838447 | G | A | 4 | CED82575 |
| X₁₄₀₁ | M2 | 33 | 1848935 | C | T | 1 | CED82579 |
| X₁₄₀₂ | M2 | 33 | 1860278 | G | A | 3 | CED82582 |
| X₁₄₀₃ | M2 | 33 | 1861675 | C | T | 2 | CED82583 |
| X₁₄₀₄ | M2 | 33 | 1887401 | C | T | 1 | CED82591 |
| X₁₄₀₅ | M2 | 33 | 1938614 | G | A | 2 | CED82612 |
| X₁₄₀₆ | M2 | 33 | 1959142 | G | A | 8 | CED82619 |
| X₁₄₀₇ | M2 | 33 | 2010230 | C | T | 2 | CED82636 |
| X₁₄₀₈ | M2 | 33 | 2013946 | C | A | 2 | CED82637 |
| X₁₄₀₉ | M2 | 33 | 2014235 | C | T | 2 | CED82637 |
| X₁₄₁₀ | M2 | 33 | 2033881 | C | T | 1 | CED82645 |
| X₁₄₁₁ | M2 | 33 | 2054662 | G | A | 1 | CED82654 |
| X₁₄₁₂ | M2 | 36 | 320 | G | A | 6 | |
| X₁₄₁₃ | M2 | 36 | 1056 | G | A | 6 | |
| X₁₄₁₄ | M2 | 36 | 1295 | C | T | 6 | |
| X₁₄₁₅ | M2 | 36 | 1461 | A | T | 6 | |
| X₁₄₁₆ | M2 | 42 | 804 | C | T | 4 | CDZ96333 |
| X₁₄₁₇ | M2 | 42 | 1559 | G | A | 4 | CDZ96333 |
| X₁₄₁₈ | M2 | 48 | 563 | C | T | 6 | |
| X₁₄₁₉ | M2 | 48 | 565 | T | C | 6 | |
| X₁₄₂₀ | M2 | 48 | 576 | G | A | 6 | |
| X₁₄₂₁ | M2 | 48 | 580 | T | A | 6 | |
| K₁₄₂₂ | M2 | 48 | 720 | A | G | 6 | |
| X₁₄₂₃ | M2 | 50 | 145 | G | A | 6 | |
| X₁₄₂₄ | M2 | 52 | 3042 | A | G | 3 | CED82664 |
| X₁₄₂₅ | M2 | 52 | 63859 | C | T | 2 | CED82686 |
| X₁₄₂₆ | M2 | 52 | 144382 | C | T | 3 | CED82709 |
| X₁₄₂₇ | M2 | 52 | 151974 | C | T | 2 | CED82712 |
| X₁₄₂₈ | M2 | 52 | 164586 | C | T | 2 | CED82716 |
| X₁₄₂₉ | M2 | 52 | 183986 | C | T | 8 | CED82722 |
| X₁₄₃₀ | M2 | 52 | 203253 | A | G | 3 | CED82730 |
| X₁₄₃₁ | M2 | 52 | 207354 | C | T | 8 | CED82730 |
| X₁₄₃₂ | M2 | 52 | 214949 | C | G | 3 | CED82731 |
| X₁₄₃₃ | M2 | 52 | 223289 | A | G | 6 | CED82732- |
| | | | | | | | CED82733 |
| X₁₄₃₄ | M2 | 52 | 241012 | A | G | 3 | CED82734 |
| X₁₄₃₅ | M2 | 52 | 268499 | G | A | 4 | CED82740 |
| X₁₄₃₆ | M2 | 52 | 284733 | G | A | 1 | CED82747 |
| X₁₄₃₇ | M2 | 52 | 316848 | C | T | 2 | CED82758 |
| X₁₄₃₈ | M2 | 52 | 344346 | G | A | 3 | CED82767 |
| X₁₄₃₉ | M2 | 52 | 358601 | G | A | 3 | CED82772 |
| X₁₄₄₀ | M2 | 52 | 360279 | C | T | 3 | CED82772 |
| X₁₄₄₁ | M2 | 52 | 390095 | G | A | 3 | CED82784 |
| X₁₄₄₂ | M2 | 52 | 395374 | G | A | 2 | CED82786 |
| X₁₄₄₃ | M2 | 52 | 398723 | C | T | 3 | CED82786 |
| X₁₄₄₄ | M2 | 52 | 398728 | C | T | 3 | CED82786 |
| X₁₄₄₅ | M2 | 52 | 516267 | C | T | 3 | CED82823 |
| X₁₄₄₆ | M2 | 52 | 552099 | G | A | 2 | CED82837 |
| X₁₄₄₇ | M2 | 52 | 552173 | G | A | 1 | CED82837 |
| X₁₄₄₈ | M2 | 52 | 571124 | G | A | 2 | CED82845 |
| X₁₄₄₉ | M2 | 52 | 629532 | A | T | 3 | CED82860 |
| X₁₄₅₀ | M2 | 52 | 679670 | G | A | 3 | CED82877 |
| X₁₄₅₁ | M2 | 52 | 700204 | G | A | 8 | CED82888 |
| X₁₄₅₂ | M2 | 52 | 725138 | C | T | 5 | CED82899 |
| X₁₄₅₃ | M2 | 52 | 746548 | G | A | 1 | CED82907 |
| X₁₄₅₄ | M2 | 52 | 749772 | G | A | 1 | CED82908 |
| X₁₄₅₅ | M2 | 52 | 758326 | C | T | 2 | CED82912 |
| X₁₄₅₆ | M2 | 52 | 817452 | T | A | 2 | CED82928 |
| X₁₄₅₇ | M2 | 52 | 818592 | A | G | 3 | CED82929 |
| X₁₄₅₈ | M2 | 52 | 837237 | C | T | 2 | CED82934 |
| X₁₄₅₉ | M2 | 52 | 840755 | C | T | 1 | CED82935 |
| X₁₄₆₀ | M2 | 52 | 849671 | G | A | 1 | CED82940 |
| X₁₄₆₁ | M2 | 52 | 861376 | C | T | 8 | CED82946 |
| X₁₄₆₂ | M2 | 52 | 868578 | C | T | 2 | CED82948 |
| X₁₄₆₃ | M2 | 52 | 870083 | G | A | 2 | CED82949 |
| X₁₄₆₄ | M2 | 52 | 890412 | C | T | 3 | CED82956 |
| X₁₄₆₅ | M2 | 52 | 928854 | C | T | 2 | CED82971 |
| X₁₄₆₆ | M2 | 52 | 938650 | G | A | 3 | CED82973 |
| X₁₄₆₇ | M2 | 52 | 967316 | A | T | 2 | CED82984 |
| X₁₄₆₈ | M2 | 52 | 967701 | T | A | 3 | CED82983 |
| X₁₄₆₉ | M2 | 52 | 1003899 | G | A | 2 | CED83001 |
| X₁₄₇₀ | M2 | 52 | 1029498 | G | A | 1 | CED83007 |
| X₁₄₇₁ | M2 | 52 | 1043011 | C | T | 1 | CED83012 |
| X₁₄₇₂ | M2 | 52 | 1067895 | C | T | 1 | CED83020 |
| X₁₄₇₃ | M2 | 52 | 1093730 | C | T | 3 | CED83025 |
| X₁₄₇₄ | M2 | 52 | 1151398 | G | A | 3 | CED83046 |
| X₁₄₇₅ | M2 | 52 | 1158103 | C | T | 3 | CED83051 |
| X₁₄₇₆ | M2 | 52 | 1219894 | C | T | 3 | CED83071 |
| X₁₄₇₇ | M2 | 52 | 1290031 | C | T | 3 | CED83096 |
| X₁₄₇₈ | M2 | 52 | 1292343 | G | A | 1 | CED83098 |
| X₁₄₇₉ | M2 | 52 | 1336560 | G | A | 2 | CED83113 |
| X₁₄₈₀ | M2 | 52 | 1363357 | G | A | 3 | CED83122 |
| X₁₄₈₁ | M2 | 52 | 1363405 | C | T | 1 | CED83123 |
| X₁₄₈₂ | M2 | 52 | 1365822 | C | T | 1 | CED83124 |
| X₁₄₈₃ | M2 | 52 | 1372536 | G | T | 2 | CED83125 |
| X₁₄₈₄ | M2 | 52 | 1379155 | G | A | 2 | CED83128 |
| X₁₄₈₅ | M2 | 52 | 1381652 | C | T | 1 | CED83129 |
| X₁₄₈₆ | M2 | 52 | 1392238 | C | T | 3 | CED83130 |
| X₁₄₈₇ | M2 | 52 | 1399439 | G | A | 2 | CED83134 |
| X₁₄₈₈ | M2 | 52 | 1465985 | G | A | 3 | CED83159 |
| X₁₄₈₉ | M2 | 52 | 1509004 | G | A | 2 | CED83177 |
| X₁₄₉₀ | M2 | 52 | 1526796 | G | A | 2 | CED83181 |
| X₁₄₉₁ | M2 | 52 | 1562633 | C | T | 2 | CED83195 |
| X₁₄₉₂ | M2 | 52 | 1583655 | C | T | 2 | CED83206 |
| X₁₄₉₃ | M2 | 52 | 1688685 | C | T | 2 | CED83243 |
| X₁₄₉₄ | M2 | 52 | 1708900 | A | G | 3 | CED83249 |
| X₁₄₉₅ | M2 | 52 | 1708985 | C | T | 8 | CED83248 |
| X₁₄₉₆ | M2 | 52 | 1715136 | C | T | 8 | CED83250 |
| X₁₄₉₇ | M2 | 52 | 1716210 | C | T | 3 | CED83250 |
| X₁₄₉₈ | M2 | 52 | 1760440 | G | A | 2 | CED83266 |
| X₁₄₉₉ | M2 | 52 | 1793370 | G | A | 3 | CED83275 |
| X₁₅₀₀ | M2 | 52 | 1796780 | G | A | 3 | CED83279 |
| X₁₅₀₁ | M2 | 52 | 1807309 | G | A | 3 | CED83283 |
| X₁₅₀₂ | M2 | 52 | 1809897 | G | A | 3 | CED83285 |
| X₁₅₀₃ | M2 | 52 | 1826635 | G | A | 1 | CED83289 |
| X₁₅₀₄ | M2 | 52 | 1833707 | C | A | 3 | CED83292 |
| X₁₅₀₅ | M2 | 52 | 1871310 | C | T | 2 | CED83304 |
| X₁₅₀₆ | M2 | 52 | 1874150 | G | T | 3 | CED83305 |
| X₁₅₀₇ | M2 | 52 | 1888989 | C | T | 2 | CED83312 |
| X₁₅₀₈ | M2 | 52 | 1893352 | G | A | 2 | CED83313 |
| X₁₅₀₉ | M2 | 52 | 1915927 | G | A | 3 | CED83315 |
| X₁₅₁₀ | M2 | 52 | 1956479 | C | T | 3 | CED83330 |
| X₁₅₁₁ | M2 | 52 | 1963875 | G | A | 3 | CED83335 |
| X₁₅₁₂ | M2 | 52 | 1968215 | A | G | 1 | CED83335 |
| X₁₅₁₃ | M2 | 52 | 1987096 | G | A | 4 | CED83341 |
| X₁₅₁₄ | M2 | 52 | 2029893 | G | A | 3 | CED83351 |
| X₁₅₁₅ | M2 | 52 | 2041781 | C | T | 8 | CED83355 |
| X₁₅₁₆ | M2 | 52 | 2050245 | G | A | 3 | CED83359 |
| X₁₅₁₇ | M2 | 52 | 2081390 | G | A | 1 | CED83367 |
| X₁₅₁₈ | M2 | 52 | 2120665 | C | T | 4 | CED83376 |
| X₁₅₁₉ | M2 | 52 | 2136885 | A | G | 1 | CED83382 |
| X₁₅₂₀ | M2 | 52 | 2162878 | G | A | 2 | CED83392 |
| X₁₅₂₁ | M2 | 52 | 2207855 | G | A | 1 | CED83406 |
| X₁₅₂₂ | M2 | 52 | 2258192 | C | T | 2 | CED83422 |
| X₁₅₂₃ | M2 | 52 | 2265219 | C | T | 2 | CED83425 |
| X₁₅₂₄ | M2 | 52 | 2266378 | C | T | 3 | CED83423 |
| X₁₅₂₅ | M2 | 52 | 2298758 | G | A | 8 | CED83435 |
| X₁₅₂₆ | M2 | 52 | 2308404 | G | T | 3 | CED83439 |
| X₁₅₂₇ | M2 | 52 | 2308421 | T | C | 3 | CED83439 |
| X₁₅₂₈ | M2 | 52 | 2327266 | C | T | 3 | CED83445 |
| X₁₅₂₉ | M2 | 52 | 2341484 | C | T | 3 | CED83451 |
| X₁₅₃₀ | M2 | 52 | 2373120 | C | T | 2 | CED83456 |
| X₁₅₃₁ | M2 | 52 | 2400778 | C | T | 4 | CED83463 |
| X₁₅₃₂ | M2 | 52 | 2443575 | G | A | 2 | CED83476 |
| X₁₅₃₃ | M2 | 52 | 2543220 | C | T | 3 | CED83509 |
| X₁₅₃₄ | M2 | 53 | 18836 | T | A | 3 | CDZ96344 |
| X₁₅₃₅ | M2 | 53 | 26291 | G | A | 2 | CDZ96348 |
| X₁₅₃₆ | M2 | 54 | 4815 | G | A | 1 | CDZ96384 |
| X₁₅₃₇ | M2 | 54 | 4816 | A | T | 2 | CDZ96384 |
| X₁₅₃₈ | M2 | 54 | 4817 | G | C | 2 | CDZ96384 |
| X₁₅₃₉ | M2 | 54 | 4821 | G | A | 1 | CDZ96384 |
| X₁₅₄₀ | M2 | 54 | 31207 | G | A | 2 | CDZ96392 |
| X₁₅₄₁ | M2 | 54 | 87947 | G | A | 3 | CDZ96410 |
| X₁₅₄₂ | M2 | 54 | 93810 | G | A | 2 | CDZ96414 |
| X₁₅₄₃ | M2 | 54 | 106086 | G | A | 3 | CDZ96416 |
| X₁₅₄₄ | M2 | 54 | 129380 | C | T | 2 | CDZ96423 |
| X₁₅₄₅ | M2 | 54 | 194230 | G | A | 2 | CDZ96446 |
| X₁₅₄₆ | M2 | 54 | 202467 | C | T | 1 | CDZ96448 |
| X₁₅₄₇ | M2 | 54 | 206494 | G | A | 2 | CDZ96450 |
| X₁₅₄₈ | M2 | 54 | 207737 | C | T | 1 | CDZ96450 |
| X₁₅₄₉ | M2 | 54 | 237973 | C | T | 8 | CDZ96461 |
| X₁₅₅₀ | M2 | 54 | 240326 | G | A | 1 | CDZ96462 |
| X₁₅₅₁ | M2 | 54 | 245840 | G | A | 3 | CDZ96464 |
| X₁₅₅₂ | M2 | 54 | 254295 | C | T | 2 | CDZ96465 |
| X₁₅₅₃ | M2 | 54 | 274617 | A | T | 3 | CDZ96471 |
| X₁₅₅₄ | M2 | 54 | 354688 | C | T | 3 | CDZ96493 |
| X₁₅₅₅ | M2 | 54 | 359671 | G | A | 2 | CDZ96496 |
| X₁₅₅₆ | M2 | 54 | 370320 | G | A | 2 | CDZ96499 |
| X₁₅₅₇ | M2 | 54 | 391223 | A | G | 2 | CDZ96509 |
| X₁₅₅₈ | M2 | 54 | 427542 | C | T | 2 | CDZ96520 |
| X₁₅₅₉ | M2 | 54 | 431851 | C | T | 3 | CDZ96520 |
| X₁₅₆₀ | M2 | 54 | 478527 | C | T | 2 | CDZ96537 |
| X₁₅₆₁ | M2 | 54 | 509857 | G | A | 1 | CDZ96544 |
| X₁₅₆₂ | M2 | 54 | 555986 | G | A | 2 | CDZ96563 |
| X₁₅₆₃ | M2 | 54 | 583996 | G | A | 3 | CDZ96570 |
| X₁₅₆₄ | M2 | 54 | 616268 | A | G | 3 | CDZ96581 |
| X₁₅₆₅ | M2 | 54 | 733193 | T | C | 9 | CDZ96619 |
| X₁₅₆₆ | M2 | 54 | 743396 | G | A | 1 | CDZ96621 |
| X₁₅₆₇ | M2 | 58 | 93 | T | C | 6 | |
| X₁₅₆₈ | M2 | 64 | 2113 | G | A | 4 | CDZ96626 |
| X₁₅₆₉ | M2 | 67 | 111 | G | A | 6 | |
| X₁₅₇₀ | M2 | 69 | 19892 | A | C | 3 | CED83533 |
| X₁₅₇₁ | M2 | 69 | 19893 | G | A | 3 | CED83533 |
| X₁₅₇₂ | M2 | 69 | 19917 | T | G | 3 | CED83533 |
| X₁₅₇₃ | M2 | 69 | 19920 | T | C | 3 | CED83533 |
| X₁₅₇₄ | M2 | 69 | 19926 | A | G | 3 | CED83533 |
| X₁₅₇₅ | M2 | 69 | 29006 | G | A | 3 | CED83534 |
| X₁₅₇₆ | M2 | 69 | 29188 | C | T | 3 | CED83534 |
| X₁₅₇₇ | M2 | 69 | 50642 | C | T | 8 | CED83543 |
| X₁₅₇₈ | M2 | 69 | 61047 | C | T | 2 | CED83547 |
| X₁₅₇₉ | M2 | 69 | 82710 | G | A | 3 | CED83554 |
| X₁₅₈₀ | M2 | 69 | 84820 | G | A | 1 | CED83555 |
| X₁₅₈₁ | M2 | 69 | 103317 | C | T | 2 | CED83559 |
| X₁₅₈₂ | M2 | 69 | 107920 | C | T | 1 | CED83561 |
| X₁₅₈₃ | M2 | 69 | 179873 | C | T | 3 | CED83585 |
| X₁₅₈₄ | M2 | 69 | 196810 | G | A | 2 | CED83594 |
| X₁₅₈₅ | M2 | 69 | 201005 | G | A | 3 | CED83595 |
| X₁₅₈₆ | M2 | 69 | 258091 | G | A | 2 | CED83614 |
| X₁₅₈₇ | M2 | 69 | 305997 | C | T | 2 | CED83631 |
| X₁₅₈₈ | M2 | 69 | 314003 | C | T | 1 | CED83634 |
| X₁₅₈₉ | M2 | 69 | 371916 | C | T | 2 | CED83653 |
| X₁₅₉₀ | M2 | 69 | 384418 | G | A | 2 | CED83656 |
| X₁₅₉₁ | M2 | 69 | 415659 | C | T | 2 | CED83665 |
| X₁₅₉₂ | M2 | 69 | 461986 | G | A | 1 | CED83680 |
| X₁₅₉₃ | M2 | 69 | 467686 | C | T | 2 | CED83681 |
| X₁₅₉₄ | M2 | 69 | 472563 | C | T | 3 | CED83681 |
| X₁₅₉₅ | M2 | 69 | 534495 | C | T | 2 | CED83704 |
| X₁₅₉₆ | M2 | 69 | 534506 | C | A | 2 | CED83704 |
| X₁₅₉₇ | M2 | 69 | 556138 | C | T | 3 | CED83710 |
| X₁₅₉₈ | M2 | 69 | 560348 | G | A | 1 | CED83713 |
| X₁₅₉₉ | M2 | 69 | 585845 | G | A | 2 | CED83722 |
| X₁₆₀₀ | M2 | 69 | 631511 | G | A | 2 | CED83742 |
| X₁₆₀₁ | M2 | 69 | 637299 | T | C | 2 | CED83744 |
| X₁₆₀₂ | M2 | 69 | 675579 | G | A | 3 | CED83760 |
| X₁₆₀₃ | M2 | 69 | 676581 | G | A | 2 | CED83760 |
| X₁₆₀₄ | M2 | 69 | 676853 | G | A | 3 | CED83759 |
| X₁₆₀₅ | M2 | 69 | 678406 | C | T | 3 | CED83759 |
| X₁₆₀₆ | M2 | 69 | 681141 | G | A | 2 | CED83762 |
| X₁₆₀₇ | M2 | 69 | 682770 | C | T | 2 | CED83763 |
| X₁₆₀₈ | M2 | 69 | 703602 | C | T | 2 | CED83770 |
| X₁₆₀₉ | M2 | 69 | 741255 | C | T | 1 | CED83781 |
| X₁₆₁₀ | M2 | 69 | 743019 | C | T | 1 | CED83782 |
| X₁₆₁₁ | M2 | 69 | 744021 | C | T | 3 | CED83780 |
| X₁₆₁₂ | M2 | 69 | 786726 | C | T | 2 | CED83794 |
| X₁₆₁₃ | M2 | 69 | 833538 | A | T | 4 | CED83805 |
| X₁₆₁₄ | M2 | 69 | 833558 | C | T | 4 | CED83805 |
| X₁₆₁₅ | M2 | 69 | 865820 | C | T | 2 | CED83814 |
| X₁₆₁₆ | M2 | 69 | 869706 | G | A | 8 | CED83815 |
| X₁₆₁₇ | M2 | 69 | 870338 | G | A | 2 | CED83815 |
| X₁₆₁₈ | M2 | 69 | 892199 | G | A | 1 | CED83822 |
| X₁₆₁₉ | M2 | 69 | 894019 | G | A | 3 | CED83821 |
| X₁₆₂₀ | M2 | 69 | 894462 | C | T | 3 | CED83821 |
| X₁₆₂₁ | M2 | 69 | 900223 | G | C | 1 | CED83824 |
| X₁₆₂₂ | M2 | 69 | 949982 | C | T | 2 | CED83842 |
| X₁₆₂₃ | M2 | 69 | 971219 | C | T | 3 | CED83847 |
| X₁₆₂₄ | M2 | 69 | 988364 | G | A | 2 | CED83857 |
| X₁₆₂₅ | M2 | 69 | 1042212 | G | A | 2 | CED83875 |
| X₁₆₂₆ | M2 | 69 | 1054397 | C | T | 3 | CED83879 |
| X₁₆₂₇ | M2 | 69 | 1080212 | C | T | 3 | CED83889 |
| X₁₆₂₈ | M2 | 69 | 1084824 | G | A | 3 | CED83891 |
| X₁₆₂₉ | M2 | 69 | 1084835 | C | T | 3 | CED83891 |
| X₁₆₃₀ | M2 | 69 | 1084848 | A | G | 3 | CED83891 |
| X₁₆₃₁ | M2 | 69 | 1084866 | A | G | 3 | CED83891 |
| X₁₆₃₂ | M2 | 69 | 1084931 | C | T | 3 | CED83891 |
| X₁₆₃₃ | M2 | 69 | 1089068 | G | A | 2 | CED83893 |
| X₁₆₃₄ | M2 | 69 | 1102164 | C | T | 2 | CED83897 |
| X₁₆₃₅ | M2 | 69 | 1105560 | C | T | 2 | CED83899 |
| X₁₆₃₆ | M2 | 69 | 1109785 | C | T | 3 | CED83899 |
| X₁₆₃₇ | M2 | 69 | 1109813 | A | G | 3 | CED83899 |
| X₁₆₃₈ | M2 | 69 | 1109818 | A | G | 3 | CED83899 |
| X₁₆₃₉ | M2 | 69 | 1109819 | T | A | 3 | CED83899 |
| X₁₆₄₀ | M2 | 69 | 1109823 | T | C | 3 | CED83899 |
| X₁₆₄₁ | M2 | 69 | 1109830 | A | G | 3 | CED83899 |
| X₁₆₄₂ | M2 | 69 | 1109831 | C | A | 3 | CED83899 |
| X₁₆₄₃ | M2 | 69 | 1109835 | A | G | 3 | CED83899 |
| X₁₆₄₄ | M2 | 69 | 1111789 | C | T | 3 | CED83899 |
| X₁₆₄₅ | M2 | 69 | 1115486 | C | T | 2 | CED83902 |
| X₁₆₄₆ | M2 | 69 | 1153118 | G | A | 2 | CED83915 |
| X₁₆₄₇ | M2 | 69 | 1161041 | A | G | 1 | CED83918 |
| X₁₆₄₈ | M2 | 69 | 1179032 | G | A | 1 | CED83923 |
| X₁₆₄₉ | M2 | 69 | 1199155 | C | T | 8 | CED83929 |
| X₁₆₅₀ | M2 | 69 | 1212727 | G | A | 4 | CED83933 |
| X₁₆₅₁ | M2 | 69 | 1218409 | G | A | 3 | CED83936 |
| X₁₆₅₂ | M2 | 69 | 1260087 | G | A | 3 | CED83949 |
| X₁₆₅₃ | M2 | 69 | 1327216 | C | A | 5 | CED83970 |
| X₁₆₅₄ | M2 | 69 | 1342909 | G | A | 3 | CED83975 |
| X₁₆₅₅ | M2 | 69 | 1411237 | C | T | 1 | CED84001 |
| X₁₆₅₆ | M2 | 69 | 1411421 | C | T | 3 | CED83999 |
| X₁₆₅₇ | M2 | 69 | 1430120 | C | T | 2 | CED84006 |
| X₁₆₅₈ | M2 | 69 | 1530586 | C | T | 3 | CED84038 |
| X₁₆₅₉ | M2 | 69 | 1590302 | G | A | 1 | CED84059 |
| X₁₆₆₀ | M2 | 69 | 1652288 | C | T | 3 | CED84077 |
| X₁₆₆₁ | M2 | 69 | 1674261 | C | T | 1 | CED84084 |
| X₁₆₆₂ | M2 | 69 | 1688555 | C | T | 1 | CED84088 |
| X₁₆₆₃ | M2 | 69 | 1706650 | G | A | 8 | CED84095 |
| X₁₆₆₄ | M2 | 69 | 1714728 | G | A | 3 | CED84097 |
| X₁₆₆₅ | M2 | 69 | 1735172 | G | A | 2 | CED84108 |
| X₁₆₆₆ | M2 | 69 | 1790823 | C | T | 2 | CED84125 |
| X₁₆₆₇ | M2 | 69 | 1792597 | C | T | 2 | CED84126 |
| X₁₆₆₈ | M2 | 69 | 1800584 | G | A | 3 | CED84129 |
| X₁₆₆₉ | M2 | 69 | 1803813 | G | A | 3 | CED84128 |
| X₁₆₇₀ | M2 | 69 | 1842064 | C | T | 3 | CED84140 |
| X₁₆₇₁ | M2 | 69 | 1854739 | G | A | 3 | CED84145 |
| X₁₆₇₂ | M2 | 69 | 1878663 | C | T | 3 | CED84154 |
| X₁₆₇₃ | M2 | 69 | 1924621 | C | T | 3 | CED84172 |
| X₁₆₇₄ | M2 | 69 | 1950597 | C | T | 3 | CED84182 |
| X₁₆₇₅ | M2 | 69 | 1957485 | C | T | 2 | CED84187 |
| X₁₆₇₆ | M2 | 69 | 1967529 | C | T | 1 | CED84189 |
| X₁₆₇₇ | M2 | 69 | 1968848 | A | G | 3 | CED84189 |
| X₁₆₇₈ | M2 | 69 | 2001469 | C | T | 3 | CED84197 |
| X₁₆₇₉ | M2 | 69 | 2009058 | G | A | 8 | CED84201 |
| X₁₆₈₀ | M2 | 69 | 2156050 | C | T | 3 | CED84252 |
| X₁₆₈₁ | M2 | 69 | 2160207 | C | T | 1 | CED84255 |
| X₁₆₈₂ | M2 | 69 | 2219444 | G | A | 2 | CED84271 |
| X₁₆₈₃ | M2 | 69 | 2226317 | C | T | 2 | CED84272 |
| X₁₆₈₄ | M2 | 69 | 2237950 | C | T | 2 | CED84276 |
| X₁₆₈₅ | M2 | 69 | 2255001 | G | A | 2 | CED84282 |
| X₁₆₈₆ | M2 | 69 | 2264484 | G | A | 4 | CED84284 |
| X₁₆₈₇ | M2 | 69 | 2275703 | G | A | 2 | CED84290 |
| X₁₆₈₈ | M2 | 69 | 2283903 | G | A | 2 | CED84293 |
| X₁₆₈₉ | M2 | 69 | 2348437 | C | T | 2 | CED84315 |
| X₁₆₉₀ | M2 | 69 | 2366082 | C | T | 1 | CED84318 |
| X₁₆₉₁ | M2 | 69 | 2368149 | G | A | 2 | CED84320 |
| X₁₆₉₂ | M2 | 69 | 2438727 | C | T | 3 | CED84343 |
| X₁₆₉₃ | M2 | 69 | 2439763 | A | G | 3 | CED84343 |
| X₁₆₉₄ | M2 | 69 | 2483433 | G | A | 3 | CED84352 |
| X₁₆₉₅ | M2 | 70 | 22 | T | C | 6 | |
| A₁₆₉₆ | M2 | 72 | 93 | T | C | 6 | |
| X₁₆₉₇ | M2 | 72 | 584 | G | A | 6 | |
| X₁₆₉₈ | M2 | 72 | 856 | C | T | 6 | |
| X₁₆₉₉ | M2 | 72 | 1163 | C | T | 6 | |
| X₁₇₀₀ | M2 | 73 | 174 | C | T | 3 | CDZ96628 |
| X₁₇₀₁ | M2 | 77 | 18912 | A | G | 2 | CDZ96635 |
| X₁₇₀₂ | M2 | 77 | 34978 | G | T | 3 | CDZ96642 |
| X₁₇₀₃ | M2 | 77 | 39665 | G | C | 2 | CDZ96646 |
| X₁₇₀₄ | M2 | 77 | 39842 | G | A | 8 | CDZ96646 |
| X₁₇₀₅ | M2 | 77 | 49497 | G | A | 3 | CDZ96648 |
| X₁₇₀₆ | M2 | 77 | 84022 | G | A | 8 | CDZ96659 |
| X₁₇₀₇ | M2 | 78 | 1865 | C | T | 3 | CED84359 |
| X₁₇₀₈ | M2 | 78 | 6333 | G | A | 8 | CED84360 |
| X₁₇₀₉ | M2 | 78 | 16110 | C | T | 3 | CED84363 |
| X₁₇₁₀ | M2 | 78 | 21716 | C | T | 4 | CED84364 |
| X₁₇₁₁ | M2 | 78 | 26915 | C | T | 3 | CED84366 |
| X₁₇₁₂ | M2 | 78 | 66886 | G | A | 2 | CED84381 |
| X₁₇₁₃ | M2 | 78 | 89958 | C | T | 3 | CED84387 |
| X₁₇₁₄ | M2 | 78 | 127873 | G | A | 3 | CED84395 |
| X₁₇₁₅ | M2 | 78 | 158434 | G | A | 2 | CED84406 |
| X₁₇₁₆ | M2 | 78 | 238854 | C | T | 3 | CED84430 |
| X₁₇₁₇ | M2 | 78 | 258716 | A | G | 2 | CED84437 |
| X₁₇₁₈ | M2 | 78 | 263828 | T | C | 3 | CED84438 |
| X₁₇₁₉ | M2 | 78 | 263896 | T | C | 3 | CED84438 |
| X₁₇₂₀ | M2 | 78 | 281454 | C | A | 3 | CED84441 |
| X₁₇₂₁ | M2 | 78 | 298141 | C | G | 3 | CED84443 |
| X₁₇₂₂ | M2 | 78 | 299644 | G | A | 3 | CED84443 |
| X₁₇₂₃ | M2 | 78 | 301815 | T | C | 3 | CED84443 |
| X₁₇₂₄ | M2 | 78 | 302816 | T | C | 8 | CED84443 |
| X₁₇₂₅ | M2 | 78 | 311576 | G | T | 1 | CED84447 |
| X₁₇₂₆ | M2 | 78 | 328831 | C | A | 1 | CED84450 |
| X₁₇₂₇ | M2 | 78 | 333312 | A | G | m | CED84452 |
| X₁₇₂₈ | M2 | 78 | 402094 | T | A | 1 | CED84476 |
| X₁₇₂₉ | M2 | 78 | 404944 | G | A | 3 | CED84475 |
| X₁₇₃₀ | M2 | 78 | 424723 | A | G | 2 | CED84482 |
| X₁₇₃₁ | M2 | 78 | 444292 | C | T | 2 | CED84490 |
| X₁₇₃₂ | M2 | 78 | 492399 | C | T | 3 | CED84504 |
| X₁₇₃₃ | M2 | 78 | 534530 | C | T | 1 | CED84520 |
| X₁₇₃₄ | M2 | 78 | 619735 | T | C | 3 | CED84547 |
| X₁₇₃₅ | M2 | 78 | 626958 | C | A | 4 | CED84549 |
| X₁₇₃₆ | M2 | 78 | 636848 | C | T | 3 | CED84551 |
| X₁₇₃₇ | M2 | 78 | 646777 | C | T | 2 | CED84556 |
| X₁₇₃₈ | M2 | 78 | 658932 | G | A | 1 | CED84561 |
| X₁₇₃₉ | M2 | 78 | 666005 | C | T | 3 | CED84562 |
| X₁₇₄₀ | M2 | 78 | 708376 | C | T | 2 | CED84580 |
| X₁₇₄₁ | M2 | 78 | 720093 | G | A | 4 | CED84583 |
| X₁₇₄₂ | M2 | 78 | 763771 | G | A | 3 | CED84600 |
| X₁₇₄₃ | M2 | 78 | 774252 | G | A | 2 | CED84602 |
| X₁₇₄₄ | M2 | 78 | 814066 | T | C | 3 | CED84613 |
| X₁₇₄₅ | M2 | 78 | 875825 | C | T | 3 | CED84633 |
| X₁₇₄₆ | M2 | 78 | 952815 | G | A | 2 | CED84662 |
| X₁₇₄₇ | M2 | 78 | 952860 | G | A | 2 | CED84662 |
| X₁₇₄₈ | M2 | 78 | 1023381 | G | A | 2 | CED84688 |
| X₁₇₄₉ | M2 | 78 | 1032138 | G | A | 3 | CED84693 |
| X₁₇₅₀ | M2 | 78 | 1066463 | C | T | 2 | CED84704 |
| X₁₇₅₁ | M2 | 78 | 1111002 | G | A | 2 | CED84721 |
| X₁₇₅₂ | M2 | 78 | 1115511 | G | A | 1 | CED84723 |
| X₁₇₅₃ | M2 | 78 | 1118856 | C | T | 2 | CED84724 |
| X₁₇₅₄ | M2 | 78 | 1142438 | G | A | 2 | CED84729 |
| X₁₇₅₅ | M2 | 78 | 1213556 | G | A | 2 | CED84752 |
| X₁₇₅₆ | M2 | 78 | 1216573 | C | T | 1 | CED84752 |
| X₁₇₅₇ | M2 | 78 | 1302286 | C | T | 3 | CED84782 |
| X₁₇₅₈ | M2 | 78 | 1315918 | C | T | 3 | CED84786 |
| X₁₇₅₉ | M2 | 78 | 1317124 | G | A | 3 | CED84787 |
| X₁₇₆₀ | M2 | 78 | 1344640 | C | T | 3 | CED84795 |
| X₁₇₆₁ | M2 | 78 | 1394095 | T | C | 3 | CED84815 |
| X₁₇₆₂ | M2 | 78 | 1426722 | C | T | 2 | CED84829 |
| X₁₇₆₃ | M2 | 78 | 1493542 | C | T | 2 | CED84851 |
| X₁₇₆₄ | M2 | 78 | 1495995 | C | T | 1 | CED84852 |
| X₁₇₆₅ | M2 | 78 | 1532973 | G | A | 2 | CED84857 |
| X₁₇₆₆ | M2 | 78 | 1568326 | C | T | 2 | CED84868 |
| X₁₇₆₇ | M2 | 78 | 1621099 | C | A | 3 | CED84883 |
| X₁₇₆₈ | M2 | 78 | 1642170 | G | A | 3 | CED84889 |
| X₁₇₆₉ | M2 | 78 | 1660408 | G | A | 2 | CED84897 |
| X₁₇₇₀ | M2 | 78 | 1694684 | C | T | 2 | CED84912 |
| X₁₇₇₁ | M2 | 78 | 1705462 | G | A | 1 | CED84915 |
| X₁₇₇₂ | M2 | 78 | 1721661 | G | A | 3 | CED84921 |
| X₁₇₇₃ | M2 | 78 | 1742796 | G | A | 3 | CED84926 |
| X₁₇₇₄ | M2 | 79 | 426 | T | C | 1 | CDZ96674 |
| X₁₇₇₅ | M2 | 79 | 473 | T | C | 2 | CDZ96674 |
| X₁₇₇₆ | M2 | 79 | 474 | C | T | 1 | CDZ96674 |
| X₁₇₇₇ | M2 | 79 | 47085 | G | A | 3 | CDZ96684 |
| X₁₇₇₈ | M2 | 79 | 57804 | G | A | 2 | CDZ96687 |
| X₁₇₇₉ | M2 | 79 | 64658 | C | T | 3 | CDZ96688 |
| X₁₇₈₀ | M2 | 79 | 65402 | C | T | 3 | CDZ96688 |
| X₁₇₈₁ | M2 | 79 | 86234 | T | C | 3 | CDZ96695 |
| X₁₇₈₂ | M2 | 79 | 132683 | C | T | 2 | CDZ96713 |
| X₁₇₈₃ | M2 | 79 | 147578 | G | A | 2 | CDZ96719 |
| X₁₇₈₄ | M2 | 79 | 168708 | C | T | 8 | CDZ96727 |
| X₁₇₈₅ | M2 | 79 | 171814 | C | T | 3 | CDZ96727 |
| X₁₇₈₆ | M2 | 79 | 243722 | C | T | 1 | CDZ96755 |
| X₁₇₈₇ | M2 | 79 | 281783 | C | T | 2 | CDZ96768 |
| X₁₇₈₈ | M2 | 79 | 296092 | G | A | 1 | CDZ96772 |
| X₁₇₈₉ | M2 | 79 | 307021 | C | T | 3 | CDZ96777 |
| X₁₇₉₀ | M2 | 79 | 318883 | G | A | 3 | CDZ96779 |
| X₁₇₉₁ | M2 | 79 | 346823 | G | A | 2 | CDZ96789 |
| X₁₇₉₂ | M2 | 79 | 406184 | G | A | 3 | CDZ96808 |
| X₁₇₉₃ | M2 | 79 | 412057 | T | C | 1 | CDZ96811 |
| X₁₇₉₄ | M2 | 79 | 412661 | G | A | 2 | CDZ96811 |
| X₁₇₉₅ | M2 | 79 | 430749 | T | C | 3 | CDZ96815 |
| X₁₇₉₆ | M2 | 79 | 480327 | A | C | 2 | CDZ96837 |
| X₁₇₉₇ | M2 | 79 | 492523 | C | T | 3 | CDZ96842 |
| X₁₇₉₈ | M2 | 79 | 573293 | C | T | 3 | CDZ96868 |
| X₁₇₉₉ | M2 | 79 | 575411 | C | T | 3 | CDZ96868 |
| X₁₈₀₀ | M2 | 79 | 579674 | G | A | 1 | CDZ96871 |
| X₁₈₀₁ | M2 | 79 | 617057 | C | T | 2 | CDZ96882 |
| X₁₈₀₂ | M2 | 79 | 679140 | C | T | 2 | CDZ96904 |
| X₁₈₀₃ | M2 | 79 | 686946 | C | T | 3 | CDZ96907 |
| X₁₈₀₄ | M2 | 79 | 696174 | G | A | 4 | CDZ96911 |
| X₁₈₀₅ | M2 | 79 | 702824 | G | A | 3 | CDZ96914 |
| X₁₈₀₆ | M2 | 79 | 702827 | C | T | 3 | CDZ96914 |
| X₁₈₀₇ | M2 | 79 | 735053 | T | C | 3 | CDZ96928 |
| X₁₈₀₈ | M2 | 79 | 747717 | A | G | 3 | CDZ96929 |
| X₁₈₀₉ | M2 | 79 | 758025 | G | A | 3 | CDZ96931 |
| X₁₈₁₀ | M2 | 79 | 759403 | C | T | 3 | CDZ96931 |
| X₁₈₁₁ | M2 | 79 | 759589 | G | A | 4 | CDZ96932 |
| X₁₈₁₂ | M2 | 79 | 759659 | G | A | 2 | CDZ96931 |
| X₁₈₁₃ | M2 | 79 | 760005 | A | G | 4 | CDZ96932 |
| X₁₈₁₄ | M2 | 79 | 760430 | T | C | 2 | CDZ96932 |
| X₁₈₁₅ | M2 | 79 | 760458 | G | T | 1 | CDZ96932 |
| X₁₈₁₆ | M2 | 79 | 760541 | A | G | 2 | CDZ96932 |
| X₁₈₁₇ | M2 | 79 | 760891 | A | G | 2 | CDZ96932 |
| X₁₈₁₈ | M2 | 79 | 760904 | T | C | 2 | CDZ96932 |
| X₁₈₁₉ | M2 | 79 | 760988 | G | T | 2 | CDZ96932 |
| X₁₈₂₀ | M2 | 79 | 761056 | T | C | 3 | CDZ96932 |
| X₁₈₂₁ | M2 | 79 | 761339 | T | G | 3 | CDZ96932 |
| X₁₈₂₂ | M2 | 79 | 761352 | T | A | 3 | CDZ96932 |
| X₁₈₂₃ | M2 | 79 | 761355 | T | C | 3 | CDZ96932 |
| X₁₈₂₄ | M2 | 79 | 768537 | A | G | 4 | CDZ96933 |
| X₁₈₂₅ | M2 | 79 | 776112 | A | G | 4 | CDZ96934 |
| X₁₈₂₆ | M2 | 79 | 776150 | G | A | 4 | CDZ96934 |
| X₁₈₂₇ | M2 | 79 | 780012 | T | C | 4 | CDZ96934 |
| X₁₈₂₈ | M2 | 79 | 786064 | A | G | 3 | CDZ96934 |
| X₁₈₂₉ | M2 | 79 | 798799 | C | T | 1 | CDZ96939 |
| X₁₈₃₀ | M2 | 79 | 914302 | C | T | 3 | CDZ96977 |
| X₁₈₃₁ | M2 | 79 | 922582 | G | A | 1 | CDZ96983 |
| X₁₈₃₂ | M2 | 79 | 933234 | G | A | 3 | CDZ96987 |
| X₁₈₃₃ | M2 | 79 | 973493 | C | T | 4 | CDZ97000 |
| X₁₈₃₄ | M2 | 79 | 995732 | G | A | 1 | CDZ97006 |
| X₁₈₃₅ | M2 | 79 | 1000786 | A | G | 1 | CDZ97007 |
| X₁₈₃₆ | M2 | 79 | 1021855 | G | A | 3 | CDZ97016 |
| X₁₈₃₇ | M2 | 79 | 1044652 | G | A | 1 | CDZ97023 |
| X₁₈₃₈ | M2 | 79 | 1052968 | C | T | 3 | CDZ97024 |
| X₁₈₃₉ | M2 | 79 | 1107695 | C | T | 2 | CDZ97046 |
| X₁₈₄₀ | M2 | 79 | 1123288 | C | T | 2 | CDZ97049 |
| X₁₈₄₁ | M2 | 79 | 1140763 | G | A | 3 | CDZ97053 |
| X₁₈₄₂ | M2 | 79 | 1149291 | G | A | 3 | CDZ97056 |
| X₁₈₄₃ | M2 | 79 | 1151678 | C | T | 1 | CDZ97060 |
| X₁₈₄₄ | M2 | 79 | 1209208 | G | A | 3 | CDZ97077 |
| X₁₈₄₅ | M2 | 79 | 1229129 | G | A | 2 | CDZ97086 |
| X₁₈₄₆ | M2 | 79 | 1231740 | C | T | 2 | CDZ97087 |
| X₁₈₄₇ | M2 | 79 | 1286163 | T | C | 3 | CDZ97106 |
| X₁₈₄₈ | M2 | 79 | 1287496 | C | T | 1 | CDZ97107 |
| X₁₈₄₉ | M2 | 79 | 1302166 | G | A | 3 | CDZ97112 |
| X₁₈₅₀ | M2 | 79 | 1318034 | C | T | 1 | CDZ97117 |
| X₁₈₅₁ | M2 | 79 | 1336766 | C | T | 4 | CDZ97121 |
| X₁₈₅₂ | M2 | 79 | 1387800 | G | A | 3 | CDZ97135 |
| X₁₈₅₃ | M2 | 79 | 1394034 | G | A | 1 | CDZ97137 |
| X₁₈₅₄ | M2 | 79 | 1430954 | G | A | 2 | CDZ97152 |
| X₁₈₅₅ | M2 | 79 | 1447480 | G | A | 2 | CDZ97156 |
| X₁₈₅₆ | M2 | 79 | 1471094 | C | T | 3 | CDZ97163 |
| X₁₈₅₇ | M2 | 79 | 1472351 | C | T | 2 | CDZ97165 |
| X₁₈₅₈ | M2 | 79 | 1540205 | C | T | 2 | CDZ97186 |
| X₁₈₅₉ | M2 | 79 | 1570526 | C | T | 2 | CDZ97198 |
| X₁₈₆₀ | M2 | 79 | 1624415 | G | A | 1 | CDZ97217 |
| X₁₈₆₁ | M2 | 79 | 1674448 | G | A | 4 | CDZ97228 |
| X₁₈₆₂ | M2 | 79 | 1694516 | C | T | 3 | CDZ97233 |
| X₁₈₆₃ | M2 | 79 | 1724009 | G | A | 2 | CDZ97243 |
| X₁₈₆₄ | M2 | 79 | 1788096 | G | A | 2 | CDZ97262 |
| X₁₈₆₅ | M2 | 79 | 1797660 | C | T | 3 | CDZ97265 |
| X₁₈₆₆ | M2 | 79 | 1797676 | G | A | 3 | CDZ97265 |
| X₁₈₆₇ | M2 | 79 | 1842448 | G | A | 2 | CDZ97282 |
| X₁₈₆₈ | M2 | 79 | 1842834 | G | A | 2 | CDZ97282 |
| X₁₈₆₉ | M2 | 79 | 1855037 | A | G | 3 | CDZ97285 |
| X₁₈₇₀ | M2 | 79 | 1855421 | G | A | 3 | CDZ97285 |
| X₁₈₇₁ | M2 | 79 | 1859220 | A | G | 1 | CDZ97291 |
| X₁₈₇₂ | M2 | 79 | 1869889 | C | T | 3 | CDZ97293 |
| X₁₈₇₃ | M2 | 79 | 1912360 | G | A | 2 | CDZ97307 |
| X₁₈₇₄ | M2 | 79 | 1977783 | G | A | 3 | CDZ97330 |
| X₁₈₇₅ | M2 | 79 | 2001789 | C | T | 2 | CDZ97339 |
| X₁₈₇₆ | M2 | 79 | 2002072 | C | T | 1 | CDZ97339 |
| X₁₈₇₇ | M2 | 79 | 2013361 | C | T | 8 | CDZ97344 |
| X₁₈₇₈ | M2 | 79 | 2024243 | G | A | 2 | CDZ97349 |
| X₁₈₇₉ | M2 | 79 | 2027861 | C | T | 8 | CDZ97350 |
| X₁₈₈₀ | M2 | 79 | 2066016 | C | T | 1 | CDZ97366 |
| X₁₈₈₁ | M2 | 79 | 2115870 | G | A | 1 | CDZ97380 |
| X₁₈₈₂ | M2 | 79 | 2121041 | G | A | 3 | CDZ97380 |
| X₁₈₈₃ | M2 | 79 | 2152573 | C | T | 2 | CDZ97392 |
| X₁₈₈₄ | M2 | 79 | 2187306 | C | T | 3 | CDZ97404 |
| X₁₈₈₅ | M2 | 79 | 2188410 | G | A | 2 | CDZ97405 |
| X₁₈₈₆ | M2 | 79 | 2268540 | C | T | 1 | CDZ97435 |
| X₁₈₈₇ | M2 | 79 | 2293228 | C | G | 3 | CDZ97442 |
| X₁₈₈₈ | M2 | 79 | 2317043 | G | A | 1 | CDZ97452 |
| X₁₈₈₉ | M2 | 79 | 2320060 | G | A | 2 | CDZ97454 |
| X₁₈₉₀ | M2 | 79 | 2323713 | C | T | 2 | CDZ97455 |
| X₁₈₉₁ | M2 | 79 | 2339881 | C | G | 3 | CDZ97460 |
| X₁₈₉₂ | M2 | 79 | 2340326 | G | A | 3 | CDZ97462 |
| X₁₈₉₃ | M2 | 79 | 2340636 | T | C | 3 | CDZ97462 |
| X₁₈₉₄ | M2 | 85 | 421 | G | A | 6 | |
| X₁₈₉₅ | M2 | 85 | 425 | C | T | 6 | |
| X₁₈₉₆ | M2 | 85 | 462 | G | A | 6 | |
| X₁₈₉₇ | M2 | 85 | 463 | A | G | 6 | |
| X₁₈₉₈ | M2 | 85 | 467 | A | T | 6 | |
| X₁₈₉₉ | M2 | 85 | 512 | A | G | 6 | |
| X₁₉₀₀ | M2 | 85 | 516 | G | A | 6 | |
| X₁₉₀₁ | M2 | 85 | 521 | C | T | 6 | |
| X₁₉₀₂ | M2 | 85 | 522 | A | G | 6 | |
| X₁₉₀₃ | M2 | 88 | 119 | C | T | 3 | CDZ97464 |
| X₁₉₀₄ | M2 | 88 | 196 | G | A | 3 | CDZ97464 |
| X₁₉₀₅ | M2 | 88 | 898 | C | T | 4 | CDZ97464 |
| X₁₉₀₆ | M2 | 99 | 245 | G | A | 6 | |
| X₁₉₀₇ | M2 | 103 | 768 | C | T | 6 | |
| X₁₉₀₈ | M2 | 106 | 278 | A | G | 6 | |
| X₁₉₀₉ | M2 | 109 | 396 | T | C | 6 | |
| X₁₉₁₀ | M2 | 115 | 361 | G | A | 6 | |
| X₁₉₁₁ | M2 | 118 | 396 | C | T | 2 | CDZ97478 |
| X₁₉₁₂ | M2 | 118 | 870 | G | A | 4 | CDZ97478 |
| X₁₉₁₃ | M2 | 118 | 1491 | G | A | 4 | CDZ97478 |
| X₁₉₁₄ | M2 | 121 | 907 | T | C | 6 | |
| X₁₉₁₅ | M2 | 121 | 1010 | G | A | 6 | |
| X₁₉₁₆ | M2 | 124 | 35211 | G | A | 2 | CDZ97488 |
| X₁₉₁₇ | M2 | 124 | 72117 | G | A | 2 | CDZ97501 |
| X₁₉₁₈ | M2 | 124 | 72760 | G | A | 1 | CDZ97501 |
| X₁₉₁₉ | M2 | 124 | 94611 | G | A | 1 | CDZ97507 |
| X₁₉₂₀ | M2 | 124 | 96777 | C | T | 2 | CDZ97508 |
| X₁₉₂₁ | M2 | 124 | 138045 | C | T | 4 | CDZ97519 |
| X₁₉₂₂ | M2 | 124 | 156052 | G | A | 2 | CDZ97524 |
| X₁₉₂₃ | M2 | 124 | 206541 | G | A | 2 | CDZ97541 |
| X₁₉₂₄ | M2 | 124 | 222249 | C | T | 1 | CDZ97547 |
| X₁₉₂₅ | M2 | 125 | 35864 | T | C | 3 | CDZ97573 |
| X₁₉₂₆ | M2 | 129 | 299 | G | A | 3 | CDZ97577 |
| X₁₉₂₇ | M2 | 129 | 433 | G | A | 3 | CDZ97577 |
| X₁₉₂₈ | M2 | 129 | 856 | G | A | 2 | CDZ97577 |
| X₁₉₂₉ | M2 | 129 | 1013 | C | T | 1 | CDZ97577 |
| X₁₉₃₀ | M2 | 130 | 674 | C | T | 4 | CDZ97578 |
| X₁₉₃₁ | M2 | 141 | 15764 | C | T | 2 | CDZ97587 |
| X₁₉₃₂ | M2 | 145 | 296 | A | G | 6 | |
| X₁₉₃₃ | M2 | 145 | 704 | T | G | 6 | |
| X₁₉₃₄ | M2 | 149 | 1663 | A | G | 3 | CDZ97598 |
| X₁₉₃₅ | M2 | 149 | 2074 | T | G | 3 | CDZ97598 |
| X₁₉₃₆ | M2 | 159 | 106 | A | C | 6 | |
| X₁₉₃₇ | M2 | 162 | 70405 | A | G | 1 | CDZ97625 |
| X₁₉₃₈ | M2 | 162 | 96194 | G | A | 3 | CDZ97631 |
| X₁₉₃₉ | M2 | 162 | 143415 | G | T | 3 | CDZ97647 |
| X₁₉₄₀ | M2 | 162 | 223407 | C | T | 2 | CDZ97674 |
| X₁₉₄₁ | M2 | 162 | 238126 | C | T | 3 | CDZ97680 |
| X₁₉₄₂ | M2 | 162 | 308369 | T | C | 1 | CDZ97702 |
| X₁₉₄₃ | M2 | 162 | 321070 | C | T | 4 | CDZ97703 |
| X₁₉₄₄ | M2 | 162 | 321073 | A | G | 4 | CDZ97703 |
| X₁₉₄₅ | M2 | 162 | 321460 | C | G | 4 | CDZ97703 |
| X₁₉₄₆ | M2 | 162 | 321463 | A | C | 4 | CDZ97703 |
| X₁₉₄₇ | M2 | 162 | 322591 | G | A | 4 | CDZ97703 |
| X₁₉₄₈ | M2 | 162 | 323473 | C | T | 2 | CDZ97703 |
| X₁₉₄₉ | M2 | 162 | 323480 | T | C | 1 | CDZ97703 |
| X₁₉₅₀ | M2 | 162 | 323735 | A | G | 1 | CDZ97703 |
| X₁₉₅₁ | M2 | 162 | 324114 | T | C | 2 | CDZ97703 |
| X₁₉₅₂ | M2 | 162 | 324139 | C | T | 2 | CDZ97703 |
| X₁₉₅₃ | M2 | 162 | 324568 | A | G | 2 | CDZ97703 |
| X₁₉₅₄ | M2 | 162 | 351016 | C | T | 2 | CDZ97711 |
| X₁₉₅₅ | M2 | 162 | 360320 | G | A | 2 | CDZ97714 |
| X₁₉₅₆ | M2 | 162 | 361986 | T | C | 3 | CDZ97714 |
| X₁₉₅₇ | M2 | 162 | 398165 | C | T | 1 | CDZ97730 |
| X₁₉₅₈ | M2 | 162 | 408274 | C | T | 1 | CDZ97733 |
| X₁₉₅₉ | M2 | 162 | 414579 | C | T | 8 | CDZ97737 |
| X₁₉₆₀ | M2 | 162 | 423180 | C | T | 1 | CDZ97740 |
| X₁₉₆₁ | M2 | 162 | 424391 | C | T | 3 | CDZ97740 |
| X₁₉₆₂ | M2 | 162 | 444123 | T | C | 2 | CDZ97748 |
| X₁₉₆₃ | M2 | 162 | 453998 | G | T | 1 | CDZ97751 |
| X₁₉₆₄ | M2 | 162 | 467207 | C | T | 3 | CDZ97755 |
| X₁₉₆₅ | M2 | 162 | 469139 | G | A | 3 | CDZ97757 |
| X₁₉₆₆ | M2 | 162 | 476928 | C | T | 1 | CDZ97760 |
| X₁₉₆₇ | M2 | 162 | 480722 | G | A | 2 | CDZ97761 |
| X₁₉₆₈ | M2 | 162 | 481093 | G | A | 2 | CDZ97761 |
| X₁₉₆₉ | M2 | 162 | 487433 | G | A | 2 | CDZ97763 |
| X₁₉₇₀ | M2 | 162 | 491906 | G | A | 3 | CDZ97764 |
| X₁₉₇₁ | M2 | 162 | 492101 | G | A | 3 | CDZ97764 |
| X₁₉₇₂ | M2 | 162 | 501886 | C | T | 8 | CDZ97769 |
| X₁₉₇₃ | M2 | 162 | 541612 | G | A | 2 | CDZ97784 |
| X₁₉₇₄ | M2 | 162 | 618658 | C | T | 2 | CDZ97808 |
| X₁₉₇₅ | M2 | 162 | 632311 | G | A | 2 | CDZ97814 |
| X₁₉₇₆ | M2 | 162 | 633321 | G | A | 2 | CDZ97814 |
| X₁₉₇₇ | M2 | 162 | 633633 | G | A | 1 | CDZ97814 |
| X₁₉₇₈ | M2 | 162 | 640005 | C | T | 2 | CDZ97815 |
| X₁₉₇₉ | M2 | 162 | 680645 | G | A | 8 | CDZ97826 |
| X₁₉₈₀ | M2 | 162 | 696331 | T | A | 3 | CDZ97828 |
| X₁₉₈₁ | M2 | 162 | 703725 | G | A | 2 | CDZ97831 |
| X₁₉₈₂ | M2 | 162 | 738675 | G | A | 3 | CDZ97842 |
| X₁₉₈₃ | M2 | 162 | 738686 | C | T | 3 | CDZ97842 |
| X₁₉₈₄ | M2 | 162 | 801198 | C | T | 3 | CDZ97864 |
| X₁₉₈₅ | M2 | 162 | 808830 | G | A | 3 | CDZ97869 |
| X₁₉₈₆ | M2 | 162 | 825209 | G | A | 2 | CDZ97877 |
| X₁₉₈₇ | M2 | 162 | 871911 | C | T | 8 | CDZ97890 |
| X₁₉₈₈ | M2 | 162 | 914850 | C | T | 3 | CDZ97904 |
| X₁₉₈₉ | M2 | 162 | 971882 | G | A | 2 | CDZ97924 |
| X₁₉₉₀ | M2 | 164 | 58 | C | T | 6 | |
| X₁₉₉₁ | M2 | 164 | 62 | C | T | 6 | |
| X₁₉₉₂ | M2 | 175 | 385 | A | C | 6 | |
| X₁₉₉₃ | M2 | 185 | 758 | C | T | 4 | CDZ97958 |
| X₁₉₉₄ | M2 | 189 | 1306 | C | T | 3 | CDZ97961 |
| X₁₉₉₅ | M2 | 189 | 58121 | G | A | 3 | CDZ97976 |
| X₁₉₉₆ | M2 | 189 | 88726 | C | T | 1 | CDZ97985 |
| X₁₉₉₇ | M2 | 189 | 108326 | C | T | 3 | CDZ97991 |
| X₁₉₉₈ | M2 | 189 | 126573 | G | A | 3 | CDZ97996 |
| X₁₉₉₉ | M2 | 189 | 142919 | G | T | 4 | CDZ98002 |
| X₂₀₀₀ | M2 | 189 | 194647 | A | G | 3 | CDZ98020 |
| X₂₀₀₁ | M2 | 189 | 194665 | A | G | 3 | CDZ98020 |
| X₂₀₀₂ | M2 | 189 | 212408 | G | A | 2 | CDZ98028 |
| X₂₀₀₃ | M2 | 189 | 257670 | C | T | 1 | CDZ98041 |
| X₂₀₀₄ | M2 | 189 | 274346 | C | T | 3 | CDZ98046 |
| X₂₀₀₅ | M2 | 189 | 298487 | C | T | 1 | CDZ98056 |
| X₂₀₀₆ | M2 | 189 | 323680 | C | T | 2 | CDZ98063 |
| X₂₀₀₇ | M2 | 189 | 328678 | G | T | 3 | CDZ98063 |
| X₂₀₀₈ | M2 | 189 | 332957 | G | A | 3 | CDZ98065 |
| X₂₀₀₉ | M2 | 189 | 371247 | G | A | 5 | CDZ98078 |
| X₂₀₁₀ | M2 | 189 | 400292 | T | C | 3 | CDZ98086 |
| X₂₀₁₁ | M2 | 191 | 342 | A | G | 3 | CDZ98107 |
| X₂₀₁₂ | M2 | 191 | 347 | C | T | 3 | CDZ98107 |
| X₂₀₁₃ | M2 | 191 | 427 | C | T | 3 | CDZ98107 |
| X₂₀₁₄ | M2 | 191 | 451 | C | T | 3 | CDZ98107 |
| X₂₀₁₅ | M2 | 191 | 487 | A | G | 3 | CDZ98107 |
| X₂₀₁₆ | M2 | 197 | 624 | C | T | 3 | CDZ98111 |
| X₂₀₁₇ | M2 | 198 | 202 | C | A | 6 | |
| X₂₀₁₈ | M2 | 198 | 203 | G | A | 6 | |
| X₂₀₁₉ | M2 | 198 | 238 | C | T | 6 | |
| X₂₀₂₀ | M2 | 198 | 265 | C | T | 6 | |
| X₂₀₂₁ | M2 | 198 | 452 | G | A | 6 | |
| X₂₀₂₂ | M2 | 199 | 688 | C | T | 6 | |
| X₂₀₂₃ | M2 | 199 | 911 | C | T | 6 | |
| X₂₀₂₄ | M2 | 199 | 971 | C | T | 6 | |
| X₂₀₂₅ | M2 | 199 | 1218 | C | T | 6 | |
| X₂₀₂₆ | M2 | 202 | 171 | C | T | 6 | |
| X₂₀₂₇ | M2 | 205 | 360 | C | T | 3 | CDZ98112 |
| X₂₀₂₈ | M2 | 205 | 981 | C | T | 4 | CDZ98112 |
| X₂₀₂₉ | M2 | 208 | 117 | G | A | 6 | |
| X₂₀₃₀ | M2 | 208 | 476 | G | A | 6 | |
| X₂₀₃₁ | M2 | 208 | 616 | G | A | 6 | |
| X₂₀₃₂ | M2 | 208 | 935 | G | A | 6 | |
| X₂₀₃₃ | M2 | 209 | 312 | C | T | 8 | CDZ98113 |
| X₂₀₃₄ | M2 | 223 | 93 | G | A | 4 | CDZ98118 |
| X₂₀₃₅ | M2 | 223 | 102 | A | G | 4 | CDZ98118 |
| X₂₀₃₆ | M2 | 223 | 133 | A | G | 4 | CDZ98118 |
| X₂₀₃₇ | M2 | 223 | 150 | T | C | 4 | CDZ98118 |
| X₂₀₃₈ | M2 | 223 | 155 | A | C | 4 | CDZ98118 |
| X₂₀₃₉ | M2 | 223 | 158 | T | C | 4 | CDZ98118 |
| X₂₀₄₀ | M2 | 223 | 160 | C | T | 4 | CDZ98118 |
| X₂₀₄₁ | M2 | 223 | 175 | G | A | 4 | CDZ98118 |
| X₂₀₄₂ | M2 | 223 | 177 | A | G | 4 | CDZ98118 |
| X₂₀₄₃ | M2 | 223 | 188 | A | G | 4 | CDZ98118 |
| X₂₀₄₄ | M2 | 223 | 192 | C | A | 4 | CDZ98118 |
| X₂₀₄₅ | M2 | 223 | 194 | T | C | 4 | CDZ98118 |
| X₂₀₄₆ | M2 | 223 | 195 | T | G | 4 | CDZ98118 |
| X₂₀₄₇ | M2 | 223 | 197 | T | C | 4 | CDZ98118 |
| X₂₀₄₈ | M2 | 223 | 202 | G | C | 4 | CDZ98118 |
| X₂₀₄₉ | M2 | 223 | 205 | C | T | 4 | CDZ98118 |
| X₂₀₅₀ | M2 | 223 | 360 | C | T | 2 | CDZ98118 |
| X₂₀₅₁ | M2 | 223 | 506 | A | G | 7 | CDZ98118 |
| X₂₀₅₂ | M2 | 223 | 519 | T | C | 7 | CDZ98118 |
| X₂₀₅₃ | M2 | 224 | 27 | T | A | 6 | |
| X₂₀₅₄ | M2 | 224 | 126 | T | C | 6 | |
| X₂₀₅₅ | M2 | 224 | 130 | A | | 6 | |
| X₂₀₅₆ | M2 | 225 | 317 | C | T | 6 | |
| X₂₀₅₇ | M2 | 227 | 202 | G | A | 6 | |
| X₂₀₅₈ | M2 | 229 | 225 | G | C | 6 | |
| X₂₀₅₉ | M2 | 232 | 340 | G | A | 3 | CDZ98119 |
| X₂₀₆₀ | M2 | 232 | 1379 | C | T | 1 | CDZ98119 |
| X₂₀₆₁ | M2 | 232 | 1521 | C | T | 2 | CDZ98119 |
| X₂₀₆₂ | M2 | 232 | 1662 | G | A | 2 | CDZ98119 |
| X₂₀₆₃ | M2 | 232 | 1761 | C | T | 2 | CDZ98119 |
| X₂₀₆₄ | M2 | 232 | 2027 | C | T | 4 | CDZ98119 |
| X₂₀₆₅ | M2 | 232 | 3060 | C | T | 2 | CDZ98120 |
| X₂₀₆₆ | M2 | 232 | 3284 | C | T | 4 | CDZ98119 |
| X₂₀₆₇ | M2 | 232 | 4437 | G | A | 2 | CDZ98121 |
| X₂₀₆₈ | M2 | 232 | 4561 | C | T | 3 | CDZ98120 |
| X₂₀₆₉ | M2 | 237 | 210 | T | C | 6 | |
| X₂₀₇₀ | M2 | 242 | 7334 | C | T | 2 | CDZ98125 |
| X₂₀₇₁ | M2 | 242 | 41710 | G | A | 3 | CDZ98131 |
| X₂₀₇₂ | M2 | 242 | 50262 | G | A | 8 | CDZ98135 |
| X₂₀₇₃ | M2 | 242 | 88037 | G | A | 2 | CDZ98147 |
| X₂₀₇₄ | M2 | 242 | 106805 | G | A | 3 | CDZ98152 |
| X₂₀₇₅ | M2 | 242 | 107587 | G | A | 4 | CDZ98153 |
| X₂₀₇₆ | M2 | 242 | 147104 | G | A | 2 | CDZ98164 |
| X₂₀₇₇ | M2 | 24 | 155049 | C | T | 1 | CDZ98168 |
| X₂₀₇₈ | M2 | 242 | 179344 | A | T | 3 | CDZ98173 |
| X₂₀₇₉ | M2 | 242 | 189498 | G | A | 3 | CDZ98176 |
| X₂₀₈₀ | M2 | 242 | 194983 | C | T | 8 | CDZ98179 |
| X₂₀₈₁ | M2 | 242 | 213399 | C | T | 2 | CDZ98185 |
| X₂₀₈₂ | M2 | 242 | 213986 | G | A | 8 | CDZ98185 |
| X₂₀₈₃ | M2 | 242 | 220015 | G | A | 2 | CDZ98187 |
| X₂₀₈₄ | M2 | 242 | 227271 | C | T | 3 | CDZ98191 |
| X₂₀₈₅ | M2 | 242 | 233088 | G | A | 2 | CDZ98193 |
| X₂₀₈₆ | M2 | 242 | 309675 | G | A | 2 | CDZ98216 |
| X₂₀₈₇ | M2 | 242 | 310353 | G | A | 2 | CDZ98216 |
| X₂₀₈₈ | M2 | 242 | 404132 | G | A | 3 | CDZ98248 |
| X₂₀₈₉ | M2 | 242 | 405825 | G | A | 3 | CDZ98250 |
| X₂₀₉₀ | M2 | 242 | 412676 | G | A | 3 | CDZ98249 |
| X₂₀₉₁ | M2 | 242 | 485936 | G | A | 4 | CDZ98270 |
| X₂₀₉₂ | M2 | 242 | 492389 | G | A | 2 | CDZ98274 |
| X₂₀₉₃ | M2 | 242 | 514780 | C | T | 2 | CDZ98282 |
| X₂₀₉₄ | M2 | 242 | 542663 | G | A | 3 | CDZ98290 |
| X₂₀₉₅ | M2 | 242 | 559999 | G | A | 2 | CDZ98297 |
| X₂₀₉₆ | M2 | 242 | 577367 | G | A | 3 | CDZ98302 |
| X₂₀₉₇ | M2 | 242 | 602456 | G | A | 2 | CDZ98308 |
| X₂₀₉₈ | M2 | 242 | 602531 | G | A | 2 | CDZ98308 |
| X₂₀₉₉ | M2 | 242 | 604704 | C | T | 2 | CDZ98309 |
| X₂₁₀₀ | M2 | 246 | 329 | C | G | 2 | CDZ98328 |
| X₂₁₀₁ | M2 | 246 | 331 | T | C | 1 | CDZ98328 |
| X₂₁₀₂ | M2 | 249 | 2757 | C | T | [symbol] | CED84929 |
| X₂₁₀₃ | M2 | 249 | 2763 | A | T | [symbol] | CED84929 |
| X₂₁₀₄ | M2 | 249 | 42295 | C | T | [symbol] | CED84942 |
| X₂₁₀₅ | M2 | 249 | 72016 | C | T | [symbol] | CED84950 |
| X₂₁₀₆ | M2 | 249 | 104500 | C | T | [symbol] | CED84966 |
| X₂₁₀₇ | M2 | 249 | 110060 | C | T | [symbol] | CED84968 |
| X₂₁₀₈ | M2 | 249 | 142920 | G | A | [symbol] | CED84978 |
| X₂₁₀₉ | M2 | 249 | 157550 | C | T | [symbol] | CED84983 |
| X₂₁₁₀ | M2 | 249 | 191304 | C | T | [symbol] | CED84988 |
| X₂₁₁₁ | M2 | 249 | 239775 | G | A | [symbol] | CED85004 |
| X₂₁₁₂ | M2 | 249 | 272075 | C | T | [symbol] | CED85013 |
| X₂₁₁₃ | M2 | 249 | 289154 | C | T | [symbol] | CED85018 |
| X₂₁₁₄ | M2 | 249 | 294161 | G | A | [symbol] | CED85018 |
| X₂₁₁₅ | M2 | 249 | 294853 | C | T | [symbol] | CED85019 |
| X₂₁₁₆ | M2 | 249 | 305975 | G | A | [symbol] | CED85023 |
| X₂₁₁₇ | M2 | 249 | 326271 | C | T | [symbol] | CED85029 |
| X₂₁₁₈ | M2 | 249 | 376065 | A | G | [symbol] | CED85052 |
| X₂₁₁₉ | M2 | 249 | 381260 | C | T | [symbol] | CED85054 |
| X₂₁₂₀ | M2 | 249 | 390024 | G | A | [symbol] | CED85059 |
| X₂₁₂₁ | M2 | 249 | 412062 | T | C | [symbol] | CED85064 |
| X₂₁₂₂ | M2 | 249 | 469645 | G | A | [symbol] | CED85087 |
| X₂₁₂₃ | M2 | 249 | 485782 | C | T | [symbol] | CED85089 |
| X₂₁₂₄ | M2 | 249 | 487588 | G | A | [symbol] | CED85090 |
| X₂₁₂₅ | M2 | 249 | 500195 | C | T | [symbol] | CED85093 |
| X₂₁₂₆ | M2 | 249 | 501310 | C | T | [symbol] | CED85093 |
| X₂₁₂₇ | M2 | 249 | 501510 | T | C | [symbol] | CED85093 |
| X₂₁₂₈ | M2 | 249 | 532943 | A | G | [symbol] | CED85099 |
| X₂₁₂₉ | M2 | 249 | 561512 | C | A | [symbol] | CED85112 |
| X₂₁₃₀ | M2 | 249 | 561605 | C | T | [symbol] | CED85112 |
| X₂₁₃₁ | M2 | 249 | 568518 | C | T | [symbol] | CED85115 |
| X₂₁₃₂ | M2 | 249 | 598579 | C | T | [symbol] | CED85125 |
| X₂₁₃₃ | M2 | 249 | 626627 | G | A | [symbol] | CED85132 |
| X₂₁₃₄ | M2 | 249 | 637944 | G | A | [symbol] | CED85135 |
| X₂₁₃₅ | M2 | 249 | 657434 | C | T | [symbol] | CED85143 |
| X₂₁₃₆ | M2 | 249 | 660408 | G | A | [symbol] | CED85144 |
| X₂₁₃₇ | M2 | 249 | 662251 | A | T | [symbol] | CED85144 |
| X₂₁₃₈ | M2 | 249 | 746655 | C | T | [symbol] | CED85172 |
| X₂₁₃₉ | M2 | 249 | 772481 | G | A | [symbol] | CED85180 |
| X₂₁₄₀ | M2 | 249 | 849958 | C | T | [symbol] | CED85201 |
| X₂₁₄₁ | M2 | 249 | 854220 | G | A | [symbol] | CED85201 |
| X₂₁₄₂ | M2 | 249 | 911274 | C | T | [symbol] | CED85218 |
| X₂₁₄₃ | M2 | 249 | 927876 | C | T | [symbol] | CED85222 |
| X₂₁₄₄ | M2 | 249 | 932606 | C | T | [symbol] | CED85223 |
| X₂₁₄₅ | M2 | 249 | 945434 | C | T | [symbol] | CED85227 |
| X₂₁₄₆ | M2 | 249 | 945727 | C | T | [symbol] | CED85227 |
| X₂₁₄₇ | M2 | 249 | 969333 | G | T | [symbol] | CED85238 |
| X₂₁₄₈ | M2 | 249 | 972975 | G | A | [symbol] | CED85240 |
| X₂₁₄₉ | M2 | 249 | 977170 | C | T | [symbol] | CED85242 |
| X₂₁₅₀ | M2 | 249 | 989520 | C | T | [symbol] | CED85243 |
| X₂₁₅₁ | M2 | 249 | 1014098 | C | T | [symbol] | CED85252 |
| X₂₁₅₂ | M2 | 249 | 1050123 | C | T | [symbol] | CED85262 |
| X₂₁₅₃ | M2 | 249 | 1062282 | G | A | [symbol] | CED85266 |
| X₂₁₅₄ | M2 | 249 | 1063615 | C | T | [symbol] | CED85265 |
| X₂₁₅₅ | M2 | 249 | 1064359 | C | T | [symbol] | CED85265 |
| X₂₁₅₆ | M2 | 249 | 1067266 | G | A | [symbol] | CED85267 |
| X₂₁₅₇ | M2 | 249 | 1072127 | C | T | [symbol] | CED85268 |
| X₂₁₅₈ | M2 | 249 | 1076166 | C | T | [symbol] | CED85270 |
| X₂₁₅₉ | M2 | 249 | 1144309 | G | A | [symbol] | CED85291 |
| X₂₁₆₀ | M2 | 249 | 1176977 | G | A | [symbol] | CED85303 |
| X₂₁₆₁ | M2 | 249 | 1195367 | G | A | [symbol] | CED85307 |
| X₂₁₆₂ | M2 | 249 | 1203953 | G | A | [symbol] | CED85311 |
| X₂₁₆₃ | M2 | 249 | 1230068 | C | T | [symbol] | CED85319 |
| X₂₁₆₄ | M2 | 249 | 1234239 | C | T | [symbol] | CED85319 |
| X₂₁₆₅ | M2 | 249 | 1263893 | C | T | [symbol] | CED85327 |
| X₂₁₆₆ | M2 | 249 | 1286733 | G | A | [symbol] | CED85336 |
| X₂₁₆₇ | M2 | 249 | 1299472 | C | T | [symbol] | CED85343 |
| X₂₁₆₈ | M2 | 249 | 1305184 | G | A | [symbol] | CED85345 |
| X₂₁₆₉ | M2 | 249 | 1359399 | G | A | [symbol] | CED85360 |
| X₂₁₇₀ | M2 | 249 | 1457529 | G | A | [symbol] | CED85398 |
| X₂₁₇₁ | M2 | 249 | 1473930 | C | T | [symbol] | CED85404 |
| X₂₁₇₂ | M2 | 249 | 1473937 | G | A | [symbol] | CED85404 |
| X₂₁₇₃ | M2 | 249 | 1475200 | G | A | [symbol] | CED85404 |
| X₂₁₇₄ | M2 | 249 | 1475590 | G | A | [symbol] | CED85404 |
| X₂₁₇₅ | M2 | 249 | 1486325 | G | A | [symbol] | CED85409 |
| X₂₁₇₆ | M2 | 249 | 1498029 | T | C | [symbol] | CED85415 |
| X₂₁₇₇ | M2 | 249 | 1502631 | G | T | [symbol] | CED85416 |
| X₂₁₇₈ | M2 | 249 | 1502641 | T | A | [symbol] | CED85416 |
| X₂₁₇₉ | M2 | 249 | 1502819 | T | G | [symbol] | CED85416 |
| X₂₁₈₀ | M2 | 249 | 1505138 | G | A | [symbol] | CED85416 |
| X₂₁₈₁ | M2 | 249 | 1505634 | T | G | [symbol] | CED85416 |
| X₂₁₈₂ | M2 | 249 | 1507424 | C | T | [symbol] | CED85418 |
| X₂₁₈₃ | M2 | 249 | 1519431 | A | G | [symbol] | CED85419 |
| X₂₁₈₄ | M2 | 249 | 1520089 | C | T | [symbol] | CED85420 |
| X₂₁₈₅ | M2 | 249 | 1520565 | C | A | [symbol] | CED85420 |
| X₂₁₈₆ | M2 | 249 | 1520566 | C | T | [symbol] | CED85420 |
| X₂₁₈₇ | M2 | 249 | 1531574 | C | T | [symbol] | CED85423 |
| X₂₁₈₈ | M2 | 249 | 1555472 | C | T | [symbol] | CED85428 |
| X₂₁₈₉ | M2 | 249 | 1567124 | G | A | [symbol] | CED85432 |
| X₂₁₉₀ | M2 | 249 | 1619036 | G | A | [symbol] | CED85450 |
| X₂₁₉₁ | M2 | 249 | 1623151 | G | A | [symbol] | CED85451 |
| X₂₁₉₂ | M2 | 249 | 1653615 | T | G | [symbol] | CED85457 |
| X₂₁₉₃ | M2 | 249 | 1653962 | C | T | [symbol] | CED85458 |
| X₂₁₉₄ | M2 | 249 | 1679109 | T | A | [symbol] | CED85461 |
| X₂₁₉₅ | M2 | 249 | 1726777 | C | T | [symbol] | CED85474 |
| X₂₁₉₆ | M2 | 249 | 1734722 | G | A | [symbol] | CED85477 |
| X₂₁₉₇ | M2 | 249 | 1737431 | C | T | [symbol] | CED85478 |
| X₂₁₉₈ | M2 | 249 | 1780551 | A | G | [symbol] | CED85493 |
| X₂₁₉₉ | M2 | 249 | 1780569 | A | G | [symbol] | CED85493 |
| X₂₂₀₀ | M2 | 249 | 1801965 | G | A | [symbol] | CED85501 |
| X₂₂₀₁ | M2 | 249 | 1803575 | C | T | [symbol] | CED85502 |
| X₂₂₀₂ | M2 | 249 | 1804835 | G | A | [symbol] | CED85502 |
| X₂₂₀₃ | M2 | 249 | 1835770 | G | A | [symbol] | CED85509 |
| X₂₂₀₄ | M2 | 249 | 1862072 | C | T | [symbol] | CED85520 |
| X₂₂₀₅ | M2 | 249 | 1880762 | G | A | [symbol] | CED85525 |
| X₂₂₀₆ | M2 | 249 | 1902250 | G | T | [symbol] | CED85535 |
| X₂₂₀₇ | M2 | 249 | 1915336 | C | T | [symbol] | CED85537 |
| X₂₂₀₈ | M2 | 249 | 1944026 | G | A | [symbol] | CED85545 |
| X₂₂₀₉ | M2 | 249 | 1951259 | G | A | [symbol] | CED85547 |
| X₂₂₁₀ | M2 | 249 | 1988505 | C | T | [symbol] | CED85560 |
| X₂₂₁₁ | M2 | 249 | 2015272 | G | A | [symbol] | CED85567 |
| X₂₂₁₂ | M2 | 249 | 2027477 | G | A | [symbol] | CED85573 |
| X₂₂₁₃ | M2 | 249 | 2032298 | C | T | [symbol] | CED85575 |
| X₂₂₁₄ | M2 | 249 | 2054064 | C | T | [symbol] | CED85583 |
| X₂₂₁₅ | M2 | 249 | 2082550 | A | G | [symbol] | CED85591 |
| X₂₂₁₆ | M2 | 249 | 2110956 | C | T | [symbol] | CED85603 |
| X₂₂₁₇ | M2 | 249 | 2131353 | C | T | [symbol] | CED85609 |
| X₂₂₁₈ | M2 | 249 | 2147501 | C | T | [symbol] | CED85612 |
| X₂₂₁₉ | M2 | 249 | 2198205 | G | A | [symbol] | CED85628 |
| X₂₂₂₀ | M2 | 249 | 2223626 | G | A | [symbol] | CED85633 |
| X₂₂₂₁ | M2 | 249 | 2254072 | G | A | [symbol] | CED85640 |
| X₂₂₂₂ | M2 | 249 | 2255547 | G | C | [symbol] | CED85640 |
| X₂₂₂₃ | M2 | 251 | 1135 | G | A | [symbol] | |
| X₂₂₂₄ | M2 | 252 | 512 | C | T | [symbol] | |
| X₂₂₂₅ | M2 | 252 | 524 | C | T | [symbol] | |
| X₂₂₂₆ | M2 | 258 | 166 | A | G | [symbol] | |
| X₂₂₂₇ | M2 | 258 | 1139 | A | T | [symbol] | |
| X₂₂₂₈ | M2 | 258 | 1394 | G | A | [symbol] | |
| X₂₂₂₉ | M2 | 259 | 91 | G | C | [symbol] | |
| X₂₂₃₀ | M2 | 260 | 156 | G | A | [symbol] | |
| X₂₂₃₁ | M2 | 262 | 4049 | G | A | [symbol] | CDZ98330 |
| X₂₂₃₂ | M2 | 262 | 12289 | C | T | [symbol] | CDZ98333 |
| X₂₂₃₃ | M2 | 262 | 16863 | C | T | [symbol] | CDZ98334 |
| X₂₂₃₄ | M2 | 262 | 20959 | G | A | [symbol] | CDZ98336 |
| X₂₂₃₅ | M2 | 262 | 30347 | G | A | [symbol] | CDZ98338 |
| X₂₂₃₆ | M2 | 262 | 30359 | G | A | [symbol] | CDZ98338 |
| X₂₂₃₇ | M2 | 262 | 63941 | G | A | [symbol] | CDZ98349 |
| X₂₂₃₈ | M2 | 262 | 108865 | C | T | [symbol] | CDZ98369 |
| X₂₂₃₉ | M2 | 262 | 143387 | C | T | [symbol] | CDZ98375 |
| X₂₂₄₀ | M2 | 262 | 148151 | C | T | [symbol] | CDZ98375 |
| X₂₂₄₁ | M2 | 262 | 148199 | G | A | [symbol] | CDZ98376 |
| X₂₂₄₂ | M2 | 262 | 164438 | G | A | [symbol] | CDZ98383 |
| X₂₂₄₃ | M2 | 262 | 201531 | G | A | [symbol] | CDZ98396 |
| X₂₂₄₄ | M2 | 262 | 236880 | G | A | [symbol] | CDZ98406 |
| X₂₂₄₅ | M2 | 262 | 242371 | C | T | [symbol] | CDZ98411 |
| X₂₂₄₆ | M2 | 262 | 252708 | G | A | [symbol] | CDZ98414 |
| X₂₂₄₇ | M2 | 262 | 319302 | G | A | [symbol] | CDZ98436 |
| X₂₂₄₈ | M2 | 262 | 345538 | C | T | [symbol] | CDZ98448 |
| X₂₂₄₉ | M2 | 262 | 350852 | C | T | [symbol] | CDZ98449 |
| X₂₂₅₀ | M2 | 262 | 380519 | C | T | [symbol] | CDZ98457 |
| X₂₂₅₁ | M2 | 262 | 397539 | G | A | [symbol] | CDZ98463 |
| X₂₂₅₂ | M2 | 262 | 414403 | C | T | [symbol] | CDZ98467 |
| X₂₂₅₃ | M2 | 262 | 420102 | G | A | [symbol] | CDZ98468 |
| X₂₂₅₄ | M2 | 262 | 444331 | G | A | [symbol] | CDZ98477 |
| X₂₂₅₅ | M2 | 262 | 446956 | C | T | [symbol] | CDZ98477 |
| X₂₂₅₆ | M2 | 262 | 510643 | G | A | [symbol] | CDZ98502 |
| X₂₂₅₇ | M2 | 262 | 525946 | C | T | [symbol] | CDZ98509 |
| X₂₂₅₈ | M2 | 262 | 539428 | G | A | [symbol] | CDZ98514 |
| X₂₂₅₉ | M2 | 262 | 539452 | A | G | [symbol] | CDZ98514 |
| X₂₂₆₀ | M2 | 262 | 539470 | A | G | [symbol] | CDZ98514 |
| X₂₂₆₁ | M2 | 262 | 539588 | A | G | [symbol] | CDZ98514 |
| X₂₂₆₂ | M2 | 262 | 552414 | C | T | [symbol] | CDZ98518 |
| X₂₂₆₃ | M2 | 262 | 557280 | G | A | [symbol] | CDZ98518 |
| X₂₂₆₄ | M2 | 262 | 560713 | G | A | [symbol] | CDZ98519 |
| X₂₂₆₅ | M2 | 262 | 562795 | C | T | [symbol] | CDZ98521 |
| X₂₂₆₆ | M2 | 262 | 567591 | G | A | [symbol] | CDZ98524 |
| X₂₂₆₇ | M2 | 262 | 567801 | G | A | [symbol] | CDZ98524 |
| X₂₂₆₈ | M2 | 262 | 618198 | G | A | [symbol] | CDZ98538 |
| X₂₂₆₉ | M2 | 262 | 638804 | G | C | [symbol] | CDZ98544 |
| X₂₂₇₀ | M2 | 262 | 638986 | C | G | [symbol] | CDZ98544 |
| X₂₂₇₁ | M2 | 262 | 639006 | C | T | [symbol] | CDZ98544 |
| X₂₂₇₂ | M2 | 262 | 639962 | A | G | [symbol] | CDZ98544 |
| X₂₂₇₃ | M2 | 262 | 640066 | A | G | [symbol] | CDZ98544 |
| X₂₂₇₄ | M2 | 262 | 655754 | A | T | [symbol] | CDZ98545 |
| X₂₂₇₅ | M2 | 262 | 655757 | T | C | [symbol] | CDZ98545 |
| X₂₂₇₆ | M2 | 262 | 656031 | C | A | [symbol] | CDZ98545 |
| X₂₂₇₇ | M2 | 262 | 656323 | C | G | [symbol] | CDZ98545 |
| X₂₂₇₈ | M2 | 262 | 668764 | C | T | [symbol] | CDZ98545 |
| X₂₂₇₉ | M2 | 262 | 691577 | C | T | [symbol] | CDZ98551 |
| X₂₂₈₀ | M2 | 262 | 729852 | G | A | [symbol] | CDZ98564 |
| X₂₂₈₁ | M2 | 262 | 751209 | C | T | [symbol] | CDZ98572 |
| X₂₂₈₂ | M2 | 262 | 801626 | G | A | [symbol] | CDZ98589 |
| X₂₂₈₃ | M2 | 262 | 822734 | G | A | [symbol] | CDZ98597 |
| X₂₂₈₄ | M2 | 262 | 825723 | A | G | [symbol] | CDZ98598 |
| X₂₂₈₅ | M2 | 262 | 835874 | C | T | [symbol] | CDZ98601 |
| X₂₂₈₆ | M2 | 262 | 856616 | G | A | [symbol] | CDZ98612 |
| X₂₂₈₇ | M2 | 262 | 865999 | G | A | [symbol] | CDZ98613 |
| X₂₂₈₈ | M2 | 262 | 882258 | G | A | [symbol] | CDZ98619 |
| X₂₂₈₉ | M2 | 262 | 971713 | G | A | [symbol] | CDZ98649 |
| X₂₂₉₀ | M2 | 262 | 976074 | C | T | [symbol] | CDZ98650 |
| X₂₂₉₁ | M2 | 262 | 1011054 | G | A | [symbol] | CDZ98659 |
| X₂₂₉₂ | M2 | 262 | 1044256 | G | A | [symbol] | CDZ98669 |
| X₂₂₉₃ | M2 | 262 | 1051389 | C | T | [symbol] | CDZ98670 |
| X₂₂₉₄ | M2 | 262 | 1209662 | G | A | [symbol] | CDZ98723 |
| X₂₂₉₅ | M2 | 262 | 1225705 | C | T | [symbol] | CDZ98730 |
| X₂₂₉₆ | M2 | 262 | 1226072 | C | T | [symbol] | CDZ98730 |
| X₂₂₉₇ | M2 | 262 | 1229407 | G | A | [symbol] | CDZ98732 |
| X₂₂₉₈ | M2 | 262 | 1277604 | G | A | [symbol] | CDZ98747 |
| X₂₂₉₉ | M2 | 262 | 1284428 | G | A | [symbol] | CDZ98748 |
| X₂₃₀₀ | M2 | 262 | 1305361 | G | A | [symbol] | CDZ98753 |
| X₂₃₀₁ | M2 | 262 | 1329643 | C | T | [symbol] | CDZ98760 |
| X₂₃₀₂ | M2 | 262 | 1332176 | C | T | [symbol] | CDZ98761 |
| X₂₃₀₃ | M2 | 262 | 1339924 | C | T | [symbol] | CDZ98763 |
| X₂₃₀₄ | M2 | 262 | 1351116 | A | G | [symbol] | CDZ98766 |
| X₂₃₀₅ | M2 | 262 | 1367361 | C | T | [symbol] | CDZ98772 |
| X₂₃₀₆ | M2 | 262 | 1408928 | G | A | [symbol] | CDZ98786 |
| X₂₃₀₇ | M2 | 262 | 1424293 | G | A | [symbol] | CDZ98791 |
| X₂₃₀₈ | M2 | 262 | 1455677 | G | A | [symbol] | CDZ98804 |
| X₂₃₀₉ | M2 | 262 | 1546246 | C | T | [symbol] | CDZ98833 |
| X₂₃₁₀ | M2 | 262 | 1550311 | C | T | [symbol] | CDZ98836 |
| X₂₃₁₁ | M2 | 262 | 1550456 | C | T | [symbol] | CDZ98837 |
| X₂₃₁₂ | M2 | 262 | 1615762 | G | A | [symbol] | CDZ98855 |
| X₂₃₁₃ | M2 | 262 | 1677620 | G | A | [symbol] | CDZ98873 |
| X₂₃₁₄ | M2 | 262 | 1694625 | C | T | [symbol] | CDZ98878 |
| X₂₃₁₅ | M2 | 262 | 1700623 | A | T | [symbol] | CDZ98883 |
| X₂₃₁₆ | M2 | 262 | 1716385 | G | A | [symbol] | CDZ98887 |
| X₂₃₁₇ | M2 | 266 | 85 | C | T | [symbol] | |
| X₂₃₁₈ | M2 | 266 | 252 | C | T | [symbol] | |
| X₂₃₁₉ | M2 | 267 | 93 | G | A | [symbol] | |
| X₂₃₂₀ | M2 | 267 | 94 | A | G | [symbol] | |
| X₂₃₂₁ | M2 | 267 | 133 | T | C | [symbol] | |
| X₂₃₂₂ | M2 | 267 | 134 | C | T | [symbol] | |
| X₂₃₂₃ | M2 | 267 | 201 | T | C | [symbol] | |
| X₂₃₂₄ | M2 | 267 | 208 | T | C | [symbol] | |
| X₂₃₂₅ | M2 | 267 | 237 | T | G | [symbol] | |
| X₂₃₂₆ | M2 | 267 | 254 | G | T | [symbol] | |
| X₂₃₂₇ | M2 | 267 | 256 | A | G | [symbol] | |
| X₂₃₂₈ | M2 | 267 | 257 | T | C | [symbol] | |
| X₂₃₂₉ | M3 | 22 | 1643 | C | T | [symbol] | CED80056 |
| X₂₃₃₀ | M3 | 22 | 2524 | G | A | [symbol] | CED80056 |
| X₂₃₃₁ | M3 | 28 | 236 | G | A | [symbol] | CDZ96150 |
| X₂₃₃₂ | M3 | 28 | 377 | G | A | [symbol] | CDZ96150 |
| X₂₃₃₃ | M3 | 28 | 1506 | C | T | [symbol] | CDZ96150 |
| X₂₃₃₄ | M3 | 28 | 1677 | C | T | [symbol] | CDZ96150 |
| X₂₃₃₅ | M3 | 43 | 464 | G | A | [symbol] | CDZ96151 |
| X₂₃₃₆ | M3 | 43 | 753 | C | T | [symbol] | CDZ96151 |
| X₂₃₃₇ | M3 | 43 | 757 | G | A | [symbol] | CDZ96151 |
| X₂₃₃₈ | M3 | 43 | 1527 | C | T | [symbol] | CDZ96151 |
| X₂₃₃₉ | M3 | 43 | 1748 | G | A | [symbol] | CDZ96151 |
| X₂₃₄₀ | M3 | 43 | 1928 | C | T | [symbol] | CDZ96151 |
| X₂₃₄₁ | M3 | 62 | 26 | T | A | [symbol] | CDZ96152 |
| X₂₃₄₂ | M3 | 62 | 27 | T | C | [symbol] | CDZ96152 |
| X₂₃₄₃ | M3 | 62 | 55 | T | A | [symbol] | CDZ96152 |
| X₂₃₄₄ | M3 | 62 | 403 | C | T | [symbol] | CDZ96152 |
| X₂₃₄₅ | M3 | 62 | 875 | G | A | [symbol] | CDZ96152 |
| X₂₃₄₆ | M3 | 102 | 339 | C | T | [symbol] | CDZ96153 |
| X₂₃₄₇ | M3 | 102 | 951 | G | A | [symbol] | CDZ96153 |
| X₂₃₄₈ | M3 | 102 | 1042 | G | A | [symbol] | CDZ96153 |
| X₂₃₄₉ | M3 | 102 | 2334 | G | A | [symbol] | CDZ96153 |
| X₂₃₅₀ | M3 | 102 | 2590 | C | T | [symbol] | CDZ96153 |
| X₂₃₅₁ | M3 | 102 | 3427 | C | T | [symbol] | CDZ96153 |
| X₂₃₅₂ | M3 | 176 | 358 | G | A | [symbol] | CED80058 |
| X₂₃₅₃ | M3 | 176 | 1872 | G | A | [symbol] | CED80058 |
| X₂₃₅₄ | M3 | 176 | 1980 | G | A | [symbol] | CED80058 |
| X₂₃₅₅ | M3 | 179 | 93 | A | G | [symbol] | CDZ96154 |
| X₂₃₅₆ | M3 | 179 | 96 | G | A | [symbol] | CDZ96154 |
| X₂₃₅₇ | M3 | 179 | 1525 | G | A | [symbol] | CDZ96154 |
| X₂₃₅₈ | M3 | 179 | 1780 | C | T | [symbol] | CDZ96154 |
| X₂₃₅₉ | M3 | 188 | 815 | C | T | [symbol] | CED80060 |
| X₂₃₆₀ | M3 | 188 | 1174 | C | T | [symbol] | CED80060 |
| X₂₃₆₁ | M3 | 188 | 2163 | G | A | [symbol] | CED80060 |
| X₂₃₆₂ | M3 | 188 | 2170 | G | A | [symbol] | CED80060 |
| X₂₃₆₃ | M3 | 188 | 2250 | C | T | [symbol] | CED80060 |
| X₂₃₆₄ | M3 | 247 | 102 | G | A | [symbol] | CED80061 |
| X₂₃₆₅ | M3 | 247 | 989 | C | T | [symbol] | CED80061 |
| X₂₃₆₆ | M3 | 247 | 1053 | G | A | [symbol] | CED80061 |
| X₂₃₆₇ | M3 | 4 | 781 | G | A | [symbol] | |
| X₂₃₆₈ | M3 | 7 | 160 | T | C | [symbol] | |
| X₂₃₆₉ | M3 | 8 | 1110 | C | T | [symbol] | |
| X₂₃₇₀ | M3 | 11 | 154 | A | C | [symbol] | |
| X₂₃₇₁ | M3 | 11 | 158 | C | T | [symbol] | |
| X₂₃₇₂ | M3 | 13 | 559 | C | T | [symbol] | CDZ96158 |
| X₂₃₇₃ | M3 | 15 | 183 | C | T | [symbol] | |
| X₂₃₇₄ | M3 | 24 | 513 | T | C | [symbol] | CDZ96160 |
| X₂₃₇₅ | M3 | 24 | 520 | C | G | [symbol] | CDZ96160 |
| X₂₃₇₆ | M3 | 24 | 5850 | C | T | [symbol] | CDZ96160 |
| X₂₃₇₇ | M3 | 24 | 169574 | C | T | [symbol] | CDZ96213 |
| X₂₃₇₈ | M3 | 24 | 172698 | G | A | [symbol] | CDZ96215 |
| X₂₃₇₉ | M3 | 24 | 180340 | G | A | [symbol] | CDZ96216 |
| X₂₃₈₀ | M3 | 24 | 216696 | C | T | [symbol] | CDZ96228 |
| X₂₃₈₁ | M3 | 24 | 266697 | G | A | [symbol] | CDZ96242 |
| X₂₃₈₂ | M3 | 24 | 307114 | G | A | [symbol] | CDZ96255 |
| X₂₃₈₃ | M3 | 24 | 340626 | C | T | [symbol] | CDZ96265 |
| X₂₃₈₄ | M3 | 24 | 340663 | T | C | [symbol] | CDZ96265 |
| X₂₃₈₅ | M3 | 24 | 400418 | C | T | [symbol] | CDZ96287 |
| X₂₃₈₅ | M3 | 24 | 420108 | G | A | [symbol] | CDZ96291 |
| X₂₃₈₇ | M3 | 25 | 622 | G | A | [symbol] | |
| X₂₃₈₈ | M3 | 27 | 445 | C | T | [symbol] | |
| X₂₃₈₉ | M3 | 27 | 1012 | A | C | [symbol] | |
| X₂₃₉₀ | M3 | 27 | 1013 | T | A | [symbol] | |
| X₂₃₉₁ | M3 | 32 | 158 | C | T | [symbol] | CDZ96332 |
| X₂₃₉₂ | M3 | 32 | 215 | C | T | [symbol] | CDZ96332 |
| X₂₃₉₃ | M3 | 32 | 2438 | C | T | [symbol] | CDZ96332 |
| X₂₃₉₄ | M3 | 32 | 2734 | C | T | [symbol] | CDZ96332 |
| X₂₃₉₅ | M3 | 32 | 2821 | C | T | [symbol] | CDZ96332 |
| X₂₃₉₆ | M3 | 32 | 3067 | C | T | [symbol] | CDZ96332 |
| X₂₃₉₇ | M3 | 32 | 3506 | C | T | [symbol] | CDZ96332 |
| X₂₃₉₈ | M3 | 32 | 3562 | G | A | [symbol] | CDZ96332 |
| X₂₃₉₉ | M3 | 33 | 399 | T | A | [symbol] | CED82002 |
| X₂₄₀₀ | M3 | 33 | 45488 | C | T | [symbol] | CED82012 |
| X₂₄₀₁ | M3 | 33 | 79913 | G | A | [symbol] | CED82024 |
| X₂₄₀₂ | M3 | 33 | 109397 | G | A | [symbol] | CED82038 |
| X₂₄₀₃ | M3 | 33 | 129733 | G | A | [symbol] | CED82045 |
| X₂₄₀₄ | M3 | 33 | 257786 | C | T | [symbol] | CED82084 |
| X₂₄₀₅ | M3 | 33 | 260027 | G | A | [symbol] | CED82084 |
| X₂₄₀₆ | M3 | 33 | 274627 | G | A | [symbol] | CED82091 |
| X₂₄₀₇ | M3 | 33 | 283808 | C | T | [symbol] | CED82093 |
| X₂₄₀₈ | M3 | 33 | 326016 | T | C | [symbol] | CED82108 |
| X₂₄₀₉ | M3 | 33 | 327840 | C | T | [symbol] | CED82108 |
| X₂₄₁₀ | M3 | 33 | 341890 | G | A | [symbol] | CED82113 |
| X₂₄₁₁ | M3 | 33 | 370630 | A | T | [symbol] | CED82121- |
| | | | | | | | CED82122 |
| X₂₄₁₂ | M3 | 33 | 371277 | G | A | [symbol] | CED82121- |
| | | | | | | | CED82122 |
| X₂₄₁₃ | M3 | 33 | 374131 | A | G | [symbol] | CED82121- |
| | | | | | | | CED82122 |
| X₂₄₁₄ | M3 | 33 | 374134 | A | G | [symbol] | CED82121- |
| | | | | | | | CED82122 |
| X₂₄₁₅ | M3 | 33 | 374137 | A | T | [symbol] | CED82121- |
| | | | | | | | CED82122 |
| X₂₄₁₆ | M3 | 33 | 374150 | A | C | [symbol] | CED82121- |
| | | | | | | | CED82122 |
| X₂₄₁₇ | M3 | 33 | 375339 | A | G | [symbol] | CED82121- |
| | | | | | | | CED82122 |
| X₂₄₁₈ | M3 | 33 | 375366 | T | C | [symbol] | CED82121- |
| | | | | | | | CED82122 |
| X₂₄₁₉ | M3 | 33 | 382577 | C | T | [symbol] | CED82122 |
| X₂₄₂₀ | M3 | 33 | 385319 | A | C | [symbol] | CED82122 |
| X₂₄₂₁ | M3 | 33 | 385527 | C | T | [symbol] | CED82122 |
| X₂₄₂₂ | M3 | 33 | 428329 | G | A | [symbol] | CED82136 |
| X₂₄₂₃ | M3 | 33 | 448815 | G | A | [symbol] | CED82144 |
| X₂₄₂₄ | M3 | 33 | 455451 | C | T | [symbol] | CED82146 |
| X₂₄₂₅ | M3 | 33 | 505339 | C | T | [symbol] | CED82165 |
| X₂₄₂₆ | M3 | 33 | 509127 | C | T | [symbol] | CED82166 |
| X₂₄₂₇ | M3 | 33 | 556726 | C | T | [symbol] | CED82183 |
| X₂₄₂₈ | M3 | 33 | 626521 | C | T | [symbol] | CED82203 |
| X₂₄₂₉ | M3 | 33 | 628247 | C | T | [symbol] | CED82205 |
| X₂₄₃₀ | M3 | 33 | 639242 | G | A | [symbol] | CED82208 |
| X₂₄₃₁ | M3 | 33 | 643436 | G | A | [symbol] | CED82208 |
| X₂₄₃₂ | M3 | 33 | 699423 | C | T | [symbol] | CED82227 |
| X₂₄₃₃ | M3 | 33 | 715901 | C | T | [symbol] | CED82231 |
| X₂₄₃₄ | M3 | 33 | 729705 | C | T | [symbol] | CED82234 |
| X₂₄₃₅ | M3 | 33 | 792965 | G | A | [symbol] | CED82256 |
| X₂₄₃₆ | M3 | 33 | 829825 | C | T | [symbol] | CED82271 |
| X₂₄₃₇ | M3 | 33 | 917771 | A | T | [symbol] | CED82294 |
| X₂₄₃₈ | M3 | 33 | 928554 | A | T | [symbol] | CED82297 |
| X₂₄₃₉ | M3 | 33 | 936350 | T | A | [symbol] | CED82300 |
| X₂₄₄₀ | M3 | 33 | 936356 | G | A | [symbol] | CED82300 |
| X₂₄₄₁ | M3 | 33 | 936571 | A | G | [symbol] | CED82300 |
| X₂₄₄₂ | M3 | 33 | 936600 | G | A | [symbol] | CED82300 |
| X₂₄₄₃ | M3 | 33 | 936697 | A | G | [symbol] | CED82300 |
| X₂₄₄₄ | M3 | 33 | 944373 | G | A | [symbol] | CED82301 |
| X₂₄₄₅ | M3 | 33 | 995886 | G | A | [symbol] | CED82318 |
| X₂₄₄₆ | M3 | 33 | 1049699 | G | A | [symbol] | CED82334 |
| X₂₄₄₇ | M3 | 33 | 1051104 | G | A | [symbol] | CED82334 |
| X₂₄₄₈ | M3 | 33 | 1086609 | G | A | [symbol] | CED82349 |
| X₂₄₄₉ | M3 | 33 | 1138444 | C | T | [symbol] | CED82363 |
| X₂₄₅₀ | M3 | 33 | 1165914 | G | A | [symbol] | CED82372 |
| X₂₄₅₁ | M3 | 33 | 1191727 | C | T | [symbol] | CED82379 |
| X₂₄₅₂ | M3 | 33 | 1246468 | C | T | [symbol] | CED82392 |
| X₂₄₅₃ | M3 | 33 | 1311181 | G | A | [symbol] | CED82413 |
| X₂₄₅₄ | M3 | 33 | 1324395 | G | A | [symbol] | CED82417 |
| X₂₄₅₅ | M3 | 33 | 1333932 | G | A | [symbol] | CED82421 |
| X₂₄₅₆ | M3 | 33 | 1339376 | C | T | [symbol] | CED82424 |
| X₂₄₅₇ | M3 | 33 | 1396438 | C | T | [symbol] | CED82442 |
| X₂₄₅₈ | M3 | 33 | 1467702 | C | T | [symbol] | CED82463 |
| X₂₄₅₉ | M3 | 33 | 1468155 | G | A | [symbol] | CED82464 |
| X₂₄₆₀ | M3 | 33 | 1474577 | G | A | [symbol] | CED82466 |
| X₂₄₆₁ | M3 | 33 | 1480984 | G | A | [symbol] | CED82466 |
| X₂₄₆₂ | M3 | 33 | 1499347 | G | A | [symbol] | CED82472 |
| X₂₄₆₃ | M3 | 33 | 1537493 | C | A | [symbol] | CED82485 |
| X₂₄₆₄ | M3 | 33 | 1544767 | A | C | [symbol] | CED82485- |
| | | | | | | | CED82486 |
| X₂₄₆₅ | M3 | 33 | 1544951 | C | G | [symbol] | CED82485- |
| | | | | | | | CED82486 |
| X₂₄₆₆ | M3 | 33 | 1572498 | C | T | [symbol] | CED82491 |
| X₂₄₆₇ | M3 | 33 | 1587443 | C | T | [symbol] | CED82497 |
| X₂₄₆₈ | M3 | 33 | 1596242 | C | T | [symbol] | CED82498 |
| X₂₄₆₉ | M3 | 33 | 1628197 | G | A | [symbol] | CED82510 |
| X₂₄₇₀ | M3 | 33 | 1628812 | C | T | [symbol] | CED82510 |
| X₂₄₇₁ | M3 | 33 | 1638726 | G | A | [symbol] | CED82513 |
| X₂₄₇₂ | M3 | 33 | 1642659 | G | A | [symbol] | CED82515 |
| X₂₄₇₃ | M3 | 33 | 1666039 | G | A | [symbol] | CED82521 |
| X₂₄₇₄ | M3 | 33 | 1682149 | C | T | [symbol] | CED82526 |
| X₂₄₇₅ | M3 | 33 | 1689192 | C | T | [symbol] | CED82529 |
| X₂₄₇₆ | M3 | 33 | 1700000 | G | A | [symbol] | CED82534 |
| X₂₄₇₇ | M3 | 33 | 1723348 | C | T | [symbol] | CED82539 |
| X₂₄₇₈ | M3 | 33 | 1728730 | G | A | [symbol] | CED82541 |
| X₂₄₇₉ | M3 | 33 | 1740595 | C | T | [symbol] | CED82545 |
| X₂₄₈₀ | M3 | 33 | 1780044 | G | A | [symbol] | CED82555 |
| X₂₄₈₁ | M3 | 33 | 1819902 | C | T | [symbol] | CED82571 |
| X₂₄₈₂ | M3 | 33 | 1823362 | C | T | [symbol] | CED82571 |
| X₂₄₈₃ | M3 | 33 | 1826223 | G | A | [symbol] | CED82573 |
| X₂₄₈₄ | M3 | 33 | 1841263 | G | A | [symbol] | CED82576 |
| X₂₄₈₅ | M3 | 33 | 1848935 | C | T | [symbol] | CED82579 |
| X₂₄₈₆ | M3 | 33 | 1861019 | G | A | [symbol] | CED82583 |
| X₂₄₈₇ | M3 | 33 | 1881896 | T | C | [symbol] | CED82588 |
| X₂₄₈₈ | M3 | 33 | 1886315 | G | A | [symbol] | CED82591 |
| X₂₄₈₉ | M3 | 33 | 1887401 | C | T | [symbol] | CED82591 |
| X₂₄₉₀ | M3 | 33 | 1888706 | C | T | [symbol] | CED82592 |
| X₂₄₉₁ | M3 | 33 | 1939670 | C | T | [symbol] | CED82612 |
| X₂₄₉₂ | M3 | 33 | 1950090 | G | A | [symbol] | CED82614 |
| X₂₄₉₃ | M3 | 33 | 1959142 | G | A | [symbol] | CED82619 |
| X₂₄₉₄ | M3 | 33 | 1975079 | G | A | [symbol] | CED82625 |
| X₂₄₉₅ | M3 | 33 | 2010230 | C | T | [symbol] | CED82636 |
| X₂₄₉₆ | M3 | 33 | 2014477 | C | T | [symbol] | CED82637 |
| X₂₄₉₇ | M3 | 33 | 2044014 | G | A | [symbol] | CED82647 |
| X₂₄₉₈ | M3 | 33 | 2050847 | G | A | [symbol] | CED82651 |
| X₂₄₉₉ | M3 | 36 | 320 | G | A | [symbol] | |
| X₂₅₀₀ | M3 | 36 | 1056 | G | A | [symbol] | |
| X₂₅₀₁ | M3 | 36 | 1295 | C | T | [symbol] | |
| X₂₅₀₂ | M3 | 36 | 1461 | A | T | [symbol] | |
| X₂₅₀₃ | M3 | 42 | 804 | C | T | [symbol] | CDZ96333 |
| X₂₅₀₄ | M3 | 42 | 1559 | G | A | [symbol] | CDZ96333 |
| X₂₅₀₅ | M3 | 48 | 563 | C | T | [symbol] | |
| X₂₅₀₆ | M3 | 48 | 565 | T | C | [symbol] | |
| X₂₅₀₇ | M3 | 48 | 576 | G | A | [symbol] | |
| X₂₅₀₈ | M3 | 48 | 580 | T | A | [symbol] | |
| X₂₅₀₉ | M3 | 50 | 145 | G | A | [symbol] | |
| X₂₅₁₀ | M3 | 52 | 3042 | A | G | [symbol] | CED82664 |
| X₂₅₁₁ | M3 | 52 | 63772 | C | T | [symbol] | CED82686 |
| X₂₅₁₂ | M3 | 52 | 63859 | C | T | [symbol] | CED82686 |
| X₂₅₁₃ | M3 | 52 | 123705 | C | T | [symbol] | CED82700 |
| X₂₅₁₄ | M3 | 52 | 128353 | G | A | [symbol] | CED82702 |
| X₂₅₁₅ | M3 | 52 | 144382 | C | T | [symbol] | CED82709 |
| X₂₅₁₆ | M3 | 52 | 151974 | C | T | [symbol] | CED82712 |
| X₂₅₁₇ | M3 | 52 | 183986 | C | T | [symbol] | CED82722 |
| X₂₅₁₈ | M3 | 52 | 207354 | C | T | [symbol] | CED82730 |
| X₂₅₁₉ | M3 | 52 | 211602 | G | A | [symbol] | CED82730 |
| X₂₅₂₀ | M3 | 52 | 223289 | A | G | [symbol] | CED82732- |
| | | | | | | | CED82733 |
| X₂₅₂₁ | M3 | 52 | 240075 | G | C | [symbol] | CED82734 |
| X₂₅₂₂ | M3 | 52 | 244305 | G | A | [symbol] | CED82734 |
| X₂₅₂₃ | M3 | 52 | 284733 | G | A | [symbol] | CED82747 |
| X₂₅₂₄ | M3 | 52 | 390095 | G | A | [symbol] | CED82784 |
| X₂₅₂₅ | M3 | 52 | 395374 | G | A | [symbol] | CED82786 |
| X₂₅₂₆ | M3 | 52 | 398723 | C | T | [symbol] | CED82786 |
| X₂₅₂₇ | M3 | 52 | 398728 | C | T | [symbol] | CED82786 |
| X₂₅₂₈ | M3 | 52 | 422792 | C | T | [symbol] | CED82794 |
| X₂₅₂₉ | M3 | 52 | 516267 | C | T | [symbol] | CED82823 |
| X₂₅₃₀ | M3 | 52 | 542542 | C | T | [symbol] | CED82833 |
| X₂₅₃₁ | M3 | 52 | 548788 | G | A | [symbol] | CED82835 |
| X₂₅₃₂ | M3 | 52 | 552099 | G | A | [symbol] | CED82837 |
| X₂₅₃₃ | M3 | 52 | 552173 | G | A | [symbol] | CED82837 |
| X₂₅₃₄ | M3 | 52 | 571124 | G | A | [symbol] | CED82845 |
| X₂₅₃₅ | M3 | 52 | 631479 | C | T | [symbol] | CED82860 |
| X₂₅₃₆ | M3 | 52 | 638622 | G | A | [symbol] | CED82862 |
| X₂₅₃₇ | M3 | 52 | 725138 | C | T | [symbol] | CED82899 |
| X₂₅₃₈ | M3 | 52 | 737403 | G | A | [symbol] | CED82903 |
| X₂₅₃₉ | M3 | 52 | 737419 | G | A | [symbol] | CED82903 |
| X₂₅₄₀ | M3 | 52 | 809156 | C | T | [symbol] | CED82928 |
| X₂₅₄₁ | M3 | 52 | 817452 | T | A | [symbol] | CED82928 |
| X₂₅₄₂ | M3 | 52 | 826846 | G | A | [symbol] | CED82931 |
| X₂₅₄₃ | M3 | 52 | 840755 | C | T | [symbol] | CED82935 |
| X₂₅₄₄ | M3 | 52 | 849671 | G | A | [symbol] | CED82940 |
| X₂₅₄₅ | M3 | 52 | 890412 | C | T | [symbol] | CED82956 |
| X₂₅₄₆ | M3 | 52 | 938650 | G | A | [symbol] | CED82973 |
| X₂₅₄₇ | M3 | 52 | 967316 | A | T | [symbol] | CED82984 |
| X₂₅₄₈ | M3 | 52 | 967701 | T | A | [symbol] | CED82983 |
| X₂₅₄₉ | M3 | 52 | 1001035 | G | A | [symbol] | CED82999 |
| X₂₅₅₀ | M3 | 52 | 1001299 | C | T | [symbol] | CED82998 |
| X₂₅₅₁ | M3 | 52 | 1003899 | G | A | [symbol] | CED83001 |
| X₂₅₅₂ | M3 | 52 | 1004724 | G | A | [symbol] | CED83001 |
| X₂₅₅₃ | M3 | 52 | 1029498 | G | A | [symbol] | CED83007 |
| X₂₅₅₄ | M3 | 52 | 1043011 | C | T | [symbol] | CED83012 |
| X₂₅₅₅ | M3 | 52 | 1067895 | C | T | [symbol] | CED83020 |
| X₂₅₅₆ | M3 | 52 | 1151398 | G | A | [symbol] | CED83046 |
| X₂₅₅₇ | M3 | 52 | 1158103 | C | T | [symbol] | CED83051 |
| X₂₅₅₈ | M3 | 52 | 1163067 | G | A | [symbol] | CED83051 |
| X₂₅₅₉ | M3 | 52 | 1219894 | C | T | [symbol] | CED83071 |
| X₂₅₆₀ | M3 | 52 | 1247924 | C | T | [symbol] | CED83081 |
| X₂₅₆₁ | M3 | 52 | 1291646 | C | T | [symbol] | CED83098 |
| X₂₅₆₂ | M3 | 52 | 1363357 | G | A | [symbol] | CED83122 |
| X₂₅₆₃ | M3 | 52 | 1363405 | C | T | [symbol] | CED83123 |
| X₂₅₆₄ | M3 | 52 | 1389320 | G | A | [symbol] | CED83130 |
| X₂₅₆₅ | M3 | 52 | 1399439 | G | A | [symbol] | CED83134 |
| X₂₅₆₆ | M3 | 52 | 1458571 | C | T | [symbol] | CED83158 |
| X₂₅₆₇ | M3 | 52 | 1474412 | G | A | [symbol] | CED83166 |
| X₂₅₆₈ | M3 | 52 | 1475408 | C | T | [symbol] | CED83166 |
| X₂₅₆₉ | M3 | 52 | 1509004 | G | A | [symbol] | CED83177 |
| X₂₅₇₀ | M3 | 52 | 1526796 | G | A | [symbol] | CED83181 |
| X₂₅₇₁ | M3 | 52 | 1570053 | C | T | [symbol] | CED83200 |
| X₂₅₇₂ | M3 | 52 | 1583655 | C | T | [symbol] | CED83206 |
| X₂₅₇₃ | M3 | 52 | 1688685 | C | T | [symbol] | CED83243 |
| X₂₅₇₄ | M3 | 52 | 1708900 | A | G | [symbol] | CED83249 |
| X₂₅₇₅ | M3 | 52 | 1708985 | C | T | [symbol] | CED83248 |
| X₂₅₇₆ | M3 | 52 | 1715136 | C | T | [symbol] | CED83250 |
| X₂₅₇₇ | M3 | 52 | 1716210 | C | T | [symbol] | CED83250 |
| X₂₅₇₈ | M3 | 52 | 1760440 | G | A | [symbol] | CED83266 |
| X₂₅₇₉ | M3 | 52 | 1793370 | G | A | [symbol] | CED83275 |
| X₂₅₈₀ | M3 | 52 | 1807309 | G | A | [symbol] | CED83283 |
| X₂₅₈₁ | M3 | 52 | 1871310 | C | T | [symbol] | CED83304 |
| X₂₅₈₂ | M3 | 52 | 1874150 | G | T | [symbol] | CED83305 |
| X₂₅₈₃ | M3 | 52 | 1874531 | C | T | [symbol] | CED83306 |
| X₂₅₈₄ | M3 | 52 | 1893352 | G | A | [symbol] | CED83313 |
| X₂₅₈₅ | M3 | 52 | 1987096 | G | A | [symbol] | CED83341 |
| X₂₅₈₆ | M3 | 52 | 1995167 | G | A | [symbol] | CED83342 |
| X₂₅₈₇ | M3 | 52 | 2041781 | C | T | [symbol] | CED83355 |
| X₂₅₈₈ | M3 | 52 | 2048018 | G | A | [symbol] | CED83358 |
| X₂₅₈₉ | M3 | 52 | 2050245 | G | A | [symbol] | CED83359 |
| X₂₅₉₀ | M3 | 52 | 2081390 | G | A | [symbol] | CED83367 |
| X₂₅₉₁ | M3 | 52 | 2093251 | C | T | [symbol] | CED83368 |
| X₂₅₉₂ | M3 | 52 | 2120665 | C | T | [symbol] | CED83376 |
| X₂₅₉₃ | M3 | 52 | 2136885 | A | G | [symbol] | CED83382 |
| X₂₅₉₄ | M3 | 52 | 2162878 | G | A | [symbol] | CED83392 |
| X₂₅₉₅ | M3 | 52 | 2178340 | G | A | [symbol] | CED83397 |
| X₂₅₉₆ | M3 | 52 | 2258192 | C | T | [symbol] | CED83422 |
| X₂₅₉₇ | M3 | 52 | 2265219 | C | T | [symbol] | CED83425 |
| X₂₅₉₈ | M3 | 52 | 2266378 | C | T | [symbol] | CED83423 |
| X₂₅₉₉ | M3 | 52 | 2266753 | C | T | [symbol] | CED83426 |
| X₂₆₀₀ | M3 | 52 | 2327266 | C | T | [symbol] | CED83445 |
| X₂₆₀₁ | M3 | 52 | 2338728 | G | A | [symbol] | CED83451 |
| X₂₆₀₂ | M3 | 52 | 2341484 | C | T | [symbol] | CED83451 |
| X₂₆₀₃ | M3 | 52 | 2364315 | G | A | [symbol] | CED83455 |
| X₂₆₀₄ | M3 | 52 | 2367582 | G | A | [symbol] | CED83455 |
| X₂₆₀₅ | M3 | 52 | 2373120 | C | T | [symbol] | CED83456 |
| X₂₆₀₆ | M3 | 52 | 2373426 | G | A | [symbol] | CED83456 |
| X₂₆₀₇ | M3 | 52 | 2373448 | C | T | [symbol] | CED83456 |
| X₂₆₀₈ | M3 | 52 | 2400778 | C | T | [symbol] | CED83463 |
| X₂₆₀₉ | M3 | 52 | 2488746 | G | A | [symbol] | CED83493 |
| X₂₆₁₀ | M3 | 52 | 2531584 | C | T | [symbol] | CED83507 |
| X₂₆₁₁ | M3 | 52 | 2543220 | C | T | [symbol] | CED83509 |
| X₂₆₁₂ | M3 | 53 | 26291 | G | A | [symbol] | CDZ96348 |
| X₂₆₁₃ | M3 | 53 | 55702 | C | T | [symbol] | CDZ96359 |
| X₂₆₁₄ | M3 | 53 | 69664 | C | T | [symbol] | CDZ96363 |
| X₂₆₁₅ | M3 | 53 | 110192 | C | T | [symbol] | CDZ96376 |
| X₂₆₁₆ | M3 | 54 | 4815 | G | A | [symbol] | CDZ96384 |
| X₂₆₁₇ | M3 | 54 | 4816 | A | T | [symbol] | CDZ96384 |
| X₂₆₁₈ | M3 | 54 | 4817 | G | C | [symbol] | CDZ96384 |
| X₂₆₁₉ | M3 | 54 | 4821 | G | A | [symbol] | CDZ96384 |
| X₂₆₂₀ | M3 | 54 | 16963 | C | T | [symbol] | CDZ96386 |
| X₂₆₂₁ | M3 | 54 | 60769 | G | A | [symbol] | CDZ96403 |
| X₂₆₂₂ | M3 | 54 | 72235 | G | A | [symbol] | CDZ96406 |
| X₂₆₂₃ | M3 | 54 | 87947 | G | A | [symbol] | CDZ96410 |
| X₂₆₂₄ | M3 | 54 | 106086 | G | A | [symbol] | CDZ96416 |
| X₂₆₂₅ | M3 | 54 | 114442 | G | A | [symbol] | CDZ96419 |
| X₂₆₂₆ | M3 | 54 | 119439 | C | T | [symbol] | CDZ96420 |
| X₂₆₂₇ | M3 | 54 | 142211 | A | G | [symbol] | CDZ96426 |
| X₂₆₂₈ | M3 | 54 | 194230 | G | A | [symbol] | CDZ96446 |
| X₂₆₂₉ | M3 | 54 | 202467 | C | T | [symbol] | CDZ96448 |
| X₂₆₃₀ | M3 | 54 | 206494 | G | A | [symbol] | CDZ96450 |
| X₂₆₃₁ | M3 | 54 | 222860 | G | A | [symbol] | CDZ96456 |
| X₂₆₃₂ | M3 | 54 | 258379 | G | A | [symbol] | CDZ96465 |
| X₂₆₃₃ | M3 | 54 | 274617 | A | T | [symbol] | CDZ96471 |
| X₂₆₃₄ | M3 | 54 | 296381 | G | A | [symbol] | CDZ96481 |
| X₂₆₃₅ | M3 | 54 | 316790 | T | G | [symbol] | CDZ96487 |
| X₂₆₃₆ | M3 | 54 | 357141 | G | A | [symbol] | CDZ96496 |
| X₂₆₃₇ | M3 | 54 | 370320 | G | A | [symbol] | CDZ96499 |
| X₂₆₃₈ | M3 | 54 | 391223 | A | G | [symbol] | CDZ96509 |
| X₂₆₃₉ | M3 | 54 | 402099 | G | A | [symbol] | CDZ96511 |
| X₂₆₄₀ | M3 | 54 | 404968 | C | T | [symbol] | CDZ96513 |
| X₂₆₄₁ | M3 | 54 | 407871 | T | A | [symbol] | CDZ96515 |
| X₂₆₄₂ | M3 | 54 | 413765 | G | A | [symbol] | CDZ96516 |
| X₂₆₄₃ | M3 | 54 | 427542 | C | T | [symbol] | CDZ96520 |
| X₂₆₄₄ | M3 | 54 | 431851 | C | T | [symbol] | CDZ96520 |
| X₂₆₄₅ | M3 | 54 | 478527 | C | T | [symbol] | CDZ96537 |
| X₂₆₄₆ | M3 | 54 | 509857 | G | A | [symbol] | CDZ96544 |
| X₂₆₄₇ | M3 | 54 | 537282 | T | C | [symbol] | CDZ96557 |
| X₂₆₄₈ | M3 | 54 | 576174 | G | A | [symbol] | CDZ96569 |
| X₂₆₄₉ | M3 | 54 | 616268 | A | G | [symbol] | CDZ96581 |
| X₂₆₅₀ | M3 | 54 | 647579 | C | T | [symbol] | CDZ96590 |
| X₂₆₅₁ | M3 | 54 | 733193 | T | C | [symbol] | CDZ96619 |
| X₂₆₅₂ | M3 | 54 | 743396 | G | A | [symbol] | CDZ96621 |
| X₂₆₅₃ | M3 | 54 | 746707 | G | A | [symbol] | CDZ96623 |
| X₂₆₅₄ | M3 | 58 | 93 | T | C | [symbol] | |
| X₂₆₅₅ | M3 | 64 | 2756 | A | C | [symbol] | CDZ96626 |
| X₂₆₅₆ | M3 | 64 | 2767 | G | A | [symbol] | CDZ96626 |
| X₂₆₅₇ | M3 | 64 | 2770 | C | T | [symbol] | CDZ96626 |
| X₂₆₅₈ | M3 | 64 | 2783 | C | T | [symbol] | CDZ96626 |
| X₂₆₅₉ | M3 | 67 | 111 | G | A | [symbol] | |
| X₂₆₆₀ | M3 | 69 | 29006 | G | A | [symbol] | CED83534 |
| X₂₆₆₁ | M3 | 69 | 52428 | G | A | [symbol] | CED83541 |
| X₂₆₆₂ | M3 | 69 | 82710 | G | A | [symbol] | CED83554 |
| X₂₆₆₃ | M3 | 69 | 107920 | C | T | [symbol] | CED83561 |
| X₂₆₆₄ | M3 | 69 | 148757 | C | T | [symbol] | CED83575 |
| X₂₆₆₅ | M3 | 69 | 201005 | G | A | [symbol] | CED83595 |
| X₂₆₆₆ | M3 | 69 | 201961 | G | A | [symbol] | CED83597 |
| X₂₆₆₇ | M3 | 69 | 258091 | G | A | [symbol] | CED83614 |
| X₂₆₆₈ | M3 | 69 | 314003 | C | T | [symbol] | CED83634 |
| X₂₆₆₉ | M3 | 69 | 371916 | C | T | [symbol] | CED83653 |
| X₂₆₇₀ | M3 | 69 | 384418 | G | A | [symbol] | CED83656 |
| X₂₆₇₁ | M3 | 69 | 415659 | C | T | [symbol] | CED83665 |
| X₂₆₇₂ | M3 | 69 | 431248 | G | A | [symbol] | CED83671 |
| X₂₆₇₃ | M3 | 69 | 461986 | G | A | [symbol] | CED83680 |
| X₂₆₇₄ | M3 | 69 | 585845 | G | A | [symbol] | CED83722 |
| X₂₆₇₅ | M3 | 69 | 631511 | G | A | [symbol] | CED83742 |
| X₂₆₇₆ | M3 | 69 | 637299 | T | C | [symbol] | CED83744 |
| X₂₆₇₇ | M3 | 69 | 638135 | G | A | [symbol] | CED83744 |
| X₂₆₇₈ | M3 | 69 | 675579 | G | A | [symbol] | CED83760 |
| X₂₆₇₉ | M3 | 69 | 676581 | G | A | [symbol] | CED83760 |
| X₂₆₈₀ | M3 | 69 | 676853 | G | A | [symbol] | CED83759 |
| X₂₆₈₁ | M3 | 69 | 678406 | C | T | [symbol] | CED83759 |
| X₂₆₈₂ | M3 | 69 | 777063 | G | A | [symbol] | CED83792 |
| X₂₆₈₃ | M3 | 69 | 803358 | G | A | [symbol] | CED83798 |
| X₂₆₈₄ | M3 | 69 | 865820 | C | T | [symbol] | CED83814 |
| X₂₆₈₅ | M3 | 69 | 869706 | G | A | [symbol] | CED83815 |
| X₂₆₈₆ | M3 | 69 | 870338 | G | A | [symbol] | CED83815 |
| X₂₆₈₇ | M3 | 69 | 892199 | G | A | [symbol] | CED83822 |
| X₂₆₈₈ | M3 | 69 | 894462 | C | T | [symbol] | CED83821 |
| X₂₆₈₉ | M3 | 69 | 898044 | C | T | [symbol] | CED83823 |
| X₂₆₉₀ | M3 | 69 | 949982 | C | T | [symbol] | CED83842 |
| X₂₆₉₁ | M3 | 69 | 971219 | C | T | [symbol] | CED83847 |
| X₂₆₉₂ | M3 | 69 | 988364 | G | A | [symbol] | CED83857 |
| X₂₆₉₃ | M3 | 69 | 989781 | G | A | [symbol] | CED83858 |
| X₂₆₉₄ | M3 | 69 | 1021695 | G | A | [symbol] | CED83868 |
| X₂₆₉₅ | M3 | 69 | 1042212 | G | A | [symbol] | CED83875 |
| X₂₆₉₆ | M3 | 69 | 1054397 | C | T | [symbol] | CED83879 |
| X₂₆₉₇ | M3 | 69 | 1084824 | G | A | [symbol] | CED83891 |
| X₂₆₉₈ | M3 | 69 | 1084835 | C | T | [symbol] | CED83891 |
| X₂₆₉₉ | M3 | 69 | 1084848 | A | G | [symbol] | CED83891 |
| X₂₇₀₀ | M3 | 69 | 1084866 | A | G | [symbol] | CED83891 |
| X₂₇₀₁ | M3 | 69 | 1084931 | C | T | [symbol] | CED83891 |
| X₂₇₀₂ | M3 | 69 | 1102164 | C | T | [symbol] | CED83897 |
| X₂₇₀₃ | M3 | 69 | 1109785 | C | T | [symbol] | CED83899 |
| X₂₇₀₄ | M3 | 69 | 1109813 | A | G | [symbol] | CED83899 |
| X₂₇₀₅ | M3 | 69 | 1109818 | A | G | [symbol] | CED83899 |
| X₂₇₀₆ | M3 | 69 | 1109819 | T | A | [symbol] | CED83899 |
| X₂₇₀₇ | M3 | 69 | 1109823 | T | C | [symbol] | CED83899 |
| X₂₇₀₈ | M3 | 69 | 1109830 | A | G | [symbol] | CED83899 |
| X₂₇₀₉ | M3 | 69 | 1109831 | C | A | [symbol] | CED83899 |
| X₂₇₁₀ | M3 | 69 | 1109835 | A | G | [symbol] | CED83899 |
| X₂₇₁₁ | M3 | 69 | 1192333 | G | A | [symbol] | CED83928 |
| X₂₇₁₂ | M3 | 69 | 1199155 | C | T | [symbol] | CED83929 |
| X₂₇₁₃ | M3 | 69 | 1218409 | G | A | [symbol] | CED83936 |
| X₂₇₁₄ | M3 | 69 | 1260087 | G | A | [symbol] | CED83949 |
| X₂₇₁₅ | M3 | 69 | 1342909 | G | A | [symbol] | CED83975 |
| X₂₇₁₆ | M3 | 69 | 1411237 | C | T | [symbol] | CED84001 |
| X₂₇₁₇ | M3 | 69 | 1411421 | C | T | [symbol] | CED83999 |
| X₂₇₁₈ | M3 | 69 | 1421240 | C | T | [symbol] | CED84004 |
| X₂₇₁₉ | M3 | 69 | 1430120 | C | T | [symbol] | CED84006 |
| X₂₇₂₀ | M3 | 69 | 1529637 | C | T | [symbol] | CED84038 |
| X₂₇₂₁ | M3 | 69 | 1590302 | G | A | [symbol] | CED84059 |
| X₂₇₂₂ | M3 | 69 | 1601173 | C | T | [symbol] | CED84062 |
| X₂₇₂₃ | M3 | 69 | 1652288 | C | T | [symbol] | CED84077 |
| X₂₇₂₄ | M3 | 69 | 1674261 | C | T | [symbol] | CED84084 |
| X₂₇₂₅ | M3 | 69 | 1688555 | C | T | [symbol] | CED84088 |
| X₂₇₂₆ | M3 | 69 | 1694047 | C | T | [symbol] | CED84089 |
| X₂₇₂₇ | M3 | 69 | 1714728 | G | A | [symbol] | CED84097 |
| X₂₇₂₈ | M3 | 69 | 1735172 | G | A | [symbol] | CED84108 |
| X₂₇₂₉ | M3 | 69 | 1784836 | C | T | [symbol] | CED84123 |
| X₂₇₃₀ | M3 | 69 | 1790823 | C | T | [symbol] | CED84125 |
| X₂₇₃₁ | M3 | 69 | 1792597 | C | T | [symbol] | CED84126 |
| X₂₇₃₂ | M3 | 69 | 1799816 | C | T | [symbol] | CED84128 |
| X₂₇₃₃ | M3 | 69 | 1800584 | G | A | [symbol] | CED84129 |
| X₂₇₃₄ | M3 | 69 | 1842064 | C | T | [symbol] | CED84140 |
| X₂₇₃₅ | M3 | 69 | 1854739 | G | A | [symbol] | CED84145 |
| X₂₇₃₆ | M3 | 69 | 1905120 | C | T | [symbol] | CED84166 |
| X₂₇₃₇ | M3 | 69 | 1924621 | C | T | [symbol] | CED84172 |
| X₂₇₃₈ | M3 | 69 | 1957485 | C | T | [symbol] | CED84187 |
| X₂₇₃₉ | M3 | 69 | 1967529 | C | T | [symbol] | CED84189 |
| X₂₇₄₀ | M3 | 69 | 1968848 | A | G | [symbol] | CED84189 |
| X₂₇₄₁ | M3 | 69 | 2001469 | C | T | [symbol] | CED84197 |
| X₂₇₄₂ | M3 | 69 | 2071540 | C | T | [symbol] | CED84226 |
| X₂₇₄₃ | M3 | 69 | 2156050 | C | T | [symbol] | CED84252 |
| X₂₇₄₄ | M3 | 69 | 2160207 | C | T | [symbol] | CED84255 |
| X₂₇₄₅ | M3 | 69 | 2165708 | G | A | [symbol] | CED84257 |
| X₂₇₄₆ | M3 | 69 | 2235113 | C | T | [symbol] | CED84274 |
| X₂₇₄₇ | M3 | 69 | 2260222 | C | T | [symbol] | CED84282 |
| X₂₇₄₈ | M3 | 69 | 2275703 | G | A | [symbol] | CED84290 |
| X₂₇₄₉ | M3 | 69 | 2421550 | C | T | [symbol] | CED84338 |
| X₂₇₅₀ | M3 | 69 | 2438727 | C | T | [symbol] | CED84343 |
| X₂₇₅₁ | M3 | 69 | 2483433 | G | A | [symbol] | CED84352 |
| X₂₇₅₂ | M3 | 72 | 93 | T | C | [symbol] | |
| X₂₇₅₃ | M3 | 72 | 584 | G | A | [symbol] | |
| X₂₇₅₄ | M3 | 72 | 856 | C | T | [symbol] | |
| X₂₇₅₅ | M3 | 72 | 1163 | C | T | [symbol] | |
| X₂₇₅₆ | M3 | 73 | 928 | C | T | [symbol] | CDZ96628 |
| X₂₇₅₇ | M3 | 75 | 451 | C | A | [symbol] | CDZ96630 |
| X₂₇₅₈ | M3 | 75 | 461 | T | C | [symbol] | CDZ96630 |
| X₂₇₅₉ | M3 | 77 | 7804 | T | A | [symbol] | CDZ96631 |
| X₂₇₆₀ | M3 | 77 | 18912 | A | G | [symbol] | CDZ96635 |
| X₂₇₆₁ | M3 | 77 | 34275 | A | G | [symbol] | CDZ96644 |
| X₂₇₆₂ | M3 | 77 | 34978 | G | T | [symbol] | CDZ96642 |
| X₂₇₆₃ | M3 | 77 | 37340 | A | G | [symbol] | CDZ96645 |
| X₂₇₆₄ | M3 | 77 | 39842 | G | A | [symbol] | CDZ96646 |
| X₂₇₆₅ | M3 | 77 | 65419 | G | A | [symbol] | CDZ96655 |
| X₂₇₆₆ | M3 | 77 | 84022 | G | A | [symbol] | CDZ96659 |
| X₂₇₆₇ | M3 | 78 | 1865 | C | T | [symbol] | CED84359 |
| X₂₇₆₈ | M3 | 78 | 6333 | G | A | [symbol] | CED84360 |
| X₂₇₆₉ | M3 | 78 | 26915 | C | T | [symbol] | CED84366 |
| X₂₇₇₀ | M3 | 78 | 89958 | C | T | [symbol] | CED84387 |
| X₂₇₇₁ | M3 | 78 | 158534 | G | A | [symbol] | CED84406 |
| X₂₇₇₂ | M3 | 78 | 203274 | G | A | [symbol] | CED84420 |
| X₂₇₇₃ | M3 | 78 | 238854 | C | T | [symbol] | CED84430 |
| X₂₇₇₄ | M3 | 78 | 262639 | C | T | [symbol] | CED84438 |
| X₂₇₇₅ | M3 | 78 | 262683 | G | A | [symbol] | CED84438 |
| X₂₇₇₆ | M3 | 78 | 263828 | T | C | [symbol] | CED84438 |
| X₂₇₇₇ | M3 | 78 | 263896 | T | C | [symbol] | CED84438 |
| X₂₇₇₈ | M3 | 78 | 281454 | C | A | [symbol] | CED84441 |
| X₂₇₇₉ | M3 | 78 | 292139 | A | T | [symbol] | CED84442 |
| X₂₇₈₀ | M3 | 78 | 298141 | C | G | [symbol] | CED84443 |
| X₂₇₈₁ | M3 | 78 | 299166 | G | A | [symbol] | CED84443 |
| X₂₇₈₂ | M3 | 78 | 299644 | G | A | [symbol] | CED84443 |
| X₂₇₈₃ | M3 | 78 | 333312 | A | G | [symbol] | CED84452 |
| X₂₇₈₄ | M3 | 78 | 386356 | C | T | [symbol] | CED84472 |
| X₂₇₈₅ | M3 | 78 | 415432 | G | A | [symbol] | CED84479 |
| X₂₇₈₆ | M3 | 78 | 439640 | C | T | [symbol] | CED84487 |
| X₂₇₈₇ | M3 | 78 | 440606 | C | T | [symbol] | CED84487 |
| X₂₇₈₈ | M3 | 78 | 506703 | G | A | [symbol] | CED84508 |
| X₂₇₈₉ | M3 | 78 | 587686 | G | A | [symbol] | CED84539 |
| X₂₇₉₀ | M3 | 78 | 715700 | G | A | [symbol] | CED84582 |
| X₂₇₉₁ | M3 | 78 | 718885 | C | T | [symbol] | CED84584 |
| X₂₇₉₂ | M3 | 78 | 720093 | G | A | [symbol] | CED84583 |
| X₂₇₉₃ | M3 | 78 | 763771 | G | A | [symbol] | CED84600 |
| X₂₇₉₄ | M3 | 78 | 774252 | G | A | [symbol] | CED84602 |
| X₂₇₉₅ | M3 | 78 | 875825 | C | T | [symbol] | CED84633 |
| X₂₇₉₆ | M3 | 78 | 918865 | C | T | [symbol] | CED84651 |
| X₂₇₉₇ | M3 | 78 | 939705 | G | A | [symbol] | CED84658 |
| X₂₇₉₈ | M3 | 78 | 952815 | G | A | [symbol] | CED84662 |
| X₂₇₉₉ | M3 | 78 | 952860 | G | A | [symbol] | CED84662 |
| X₂₈₀₀ | M3 | 78 | 976680 | C | T | [symbol] | CED84672 |
| X₂₈₀₁ | M3 | 78 | 976799 | C | T | [symbol] | CED84672 |
| X₂₈₀₂ | M3 | 78 | 1023381 | G | A | [symbol] | CED84688 |
| X₂₈₀₃ | M3 | 78 | 1067609 | G | A | [symbol] | CED84705 |
| X₂₈₀₄ | M3 | 78 | 1086537 | C | T | [symbol] | CED84713 |
| X₂₈₀₅ | M3 | 78 | 1108918 | C | T | [symbol] | CED84721 |
| X₂₈₀₆ | M3 | 78 | 1111002 | G | A | [symbol] | CED84721 |
| X₂₈₀₇ | M3 | 78 | 1115511 | G | A | [symbol] | CED84723 |
| X₂₈₀₈ | M3 | 78 | 1155093 | C | T | [symbol] | CED84732 |
| X₂₈₀₉ | M3 | 78 | 1213556 | G | A | [symbol] | CED84752 |
| X₂₈₁₀ | M3 | 78 | 1216573 | C | T | [symbol] | CED84752 |
| X₂₈₁₁ | M3 | 78 | 1247678 | C | T | [symbol] | CED84761 |
| X₂₈₁₂ | M3 | 78 | 1278982 | C | T | [symbol] | CED84773 |
| X₂₈₁₃ | M3 | 78 | 1300968 | C | T | [symbol] | CED84783 |
| X₂₈₁₄ | M3 | 78 | 1302286 | C | T | [symbol] | CED84782 |
| X₂₈₁₅ | M3 | 78 | 1315648 | C | T | [symbol] | CED84788 |
| X₂₈₁₆ | M3 | 78 | 1317124 | G | A | [symbol] | CED84787 |
| X₂₈₁₇ | M3 | 78 | 1344640 | C | T | [symbol] | CED84795 |
| X₂₈₁₈ | M3 | 78 | 1356873 | C | T | [symbol] | CED84800 |
| X₂₈₁₉ | M3 | 78 | 1376517 | G | A | [symbol] | CED84810 |
| X₂₈₂₀ | M3 | 78 | 1397982 | G | A | [symbol] | CED84818 |
| X₂₈₂₁ | M3 | 78 | 1406981 | C | T | [symbol] | CED84821 |
| X₂₈₂₂ | M3 | 78 | 1426722 | C | T | [symbol] | CED84829 |
| X₂₈₂₃ | M3 | 78 | 1475803 | G | A | [symbol] | CED84845 |
| X₂₈₂₄ | M3 | 78 | 1493542 | C | T | [symbol] | CED84851 |
| X₂₈₂₅ | M3 | 78 | 1568326 | C | T | [symbol] | CED84868 |
| X₂₈₂₆ | M3 | 78 | 1627007 | G | A | [symbol] | CED84885 |
| X₂₈₂₇ | M3 | 78 | 1642170 | G | A | [symbol] | CED84889 |
| X₂₈₂₈ | M3 | 78 | 1660408 | G | A | [symbol] | CED84897 |
| X₂₈₂₉ | M3 | 78 | 1674126 | G | A | [symbol] | CED84904 |
| X₂₈₃₀ | M3 | 78 | 1694684 | C | T | [symbol] | CED84912 |
| X₂₈₃₁ | M3 | 78 | 1721661 | G | A | [symbol] | CED84921 |
| X₂₈₃₂ | M3 | 78 | 1742796 | G | A | [symbol] | CED84926 |
| X₂₈₃₃ | M3 | 79 | 65 | G | A | [symbol] | CDZ96674 |
| X₂₈₃₄ | M3 | 79 | 133 | A | G | [symbol] | CDZ96674 |
| X₂₈₃₅ | M3 | 79 | 161 | T | C | [symbol] | CDZ96674 |
| X₂₈₃₆ | M3 | 79 | 36113 | C | T | [symbol] | CDZ96682 |
| X₂₈₃₇ | M3 | 79 | 57804 | G | A | [symbol] | CDZ96687 |
| X₂₈₃₈ | M3 | 79 | 64658 | C | T | [symbol] | CDZ96688 |
| X₂₈₃₉ | M3 | 79 | 71637 | C | T | [symbol] | CDZ96691 |
| X₂₈₄₀ | M3 | 79 | 132683 | C | T | [symbol] | CDZ96713 |
| X₂₈₄₁ | M3 | 79 | 168570 | C | T | [symbol] | CDZ96727 |
| X₂₈₄₂ | M3 | 79 | 255486 | C | T | [symbol] | CDZ96758 |
| X₂₈₄₃ | M3 | 79 | 307021 | C | T | [symbol] | CDZ96777 |
| X₂₈₄₄ | M3 | 79 | 324145 | C | T | [symbol] | CDZ96781 |
| X₂₈₄₅ | M3 | 79 | 345646 | G | A | [symbol] | CDZ96789 |
| X₂₈₄₆ | M3 | 79 | 346823 | G | A | [symbol] | CDZ96789 |
| X₂₈₄₇ | M3 | 79 | 394300 | C | T | [symbol] | CDZ96804 |
| X₂₈₄₈ | M3 | 79 | 409085 | C | T | [symbol] | CDZ96810 |
| X₂₈₄₉ | M3 | 79 | 492523 | C | T | [symbol] | CDZ96842 |
| X₂₈₅₀ | M3 | 79 | 509666 | C | T | [symbol] | CDZ96848 |
| X₂₈₅₁ | M3 | 79 | 575411 | C | T | [symbol] | CDZ96868 |
| X₂₈₅₂ | M3 | 79 | 579674 | G | A | [symbol] | CDZ96871 |
| X₂₈₅₃ | M3 | 79 | 604739 | G | A | [symbol] | CDZ96879 |
| X₂₈₅₄ | M3 | 79 | 617057 | C | T | [symbol] | CDZ96882 |
| X₂₈₅₅ | M3 | 79 | 679140 | C | T | [symbol] | CDZ96904 |
| X₂₈₅₆ | M3 | 79 | 686946 | C | T | [symbol] | CDZ96907 |
| X₂₈₅₇ | M3 | 79 | 696174 | G | A | [symbol] | CDZ96911 |
| X₂₈₅₈ | M3 | 79 | 735053 | T | C | [symbol] | CDZ96928 |
| X₂₈₅₉ | M3 | 79 | 747717 | A | G | [symbol] | CDZ96929 |
| X₂₈₆₀ | M3 | 79 | 748083 | G | T | [symbol] | CDZ96929 |
| X₂₈₆₁ | M3 | 79 | 758025 | G | A | [symbol] | CDZ96931 |
| X₂₈₆₂ | M3 | 79 | 758078 | T | C | [symbol] | CDZ96931 |
| X₂₈₆₃ | M3 | 79 | 759195 | T | A | [symbol] | CDZ96931 |
| X₂₈₆₄ | M3 | 79 | 759403 | C | T | [symbol] | CDZ96931 |
| X₂₈₆₅ | M3 | 79 | 759659 | G | A | [symbol] | CDZ96931 |
| X₂₈₆₆ | M3 | 79 | 760430 | T | C | [symbol] | CDZ96932 |
| X₂₈₆₇ | M3 | 79 | 760458 | G | T | [symbol] | CDZ96932 |
| X₂₈₆₈ | M3 | 79 | 760541 | A | G | [symbol] | CDZ96932 |
| X₂₈₆₉ | M3 | 79 | 760891 | A | G | [symbol] | CDZ96932 |
| X₂₈₇₀ | M3 | 79 | 760904 | T | C | [symbol] | CDZ96932 |
| X₂₈₇₁ | M3 | 79 | 761056 | T | C | [symbol] | CDZ96932 |
| X₂₈₇₂ | M3 | 79 | 768537 | A | G | [symbol] | CDZ96933 |
| X₂₈₇₃ | M3 | 79 | 771663 | T | G | [symbol] | CDZ96933 |
| X₂₈₇₄ | M3 | 79 | 786064 | A | G | [symbol] | CDZ96934 |
| X₂₈₇₅ | M3 | 79 | 798799 | C | T | [symbol] | CDZ96939 |
| X₂₈₇₆ | M3 | 79 | 864204 | C | T | [symbol] | CDZ96959 |
| X₂₈₇₇ | M3 | 79 | 874161 | G | A | [symbol] | CDZ96965 |
| X₂₈₇₈ | M3 | 79 | 933234 | G | A | [symbol] | CDZ96987 |
| X₂₈₇₉ | M3 | 79 | 940003 | C | T | [symbol] | CDZ96991 |
| X₂₈₈₀ | M3 | 79 | 1000786 | A | G | [symbol] | CDZ97007 |
| X₂₈₈₁ | M3 | 79 | 1044652 | G | A | [symbol] | CDZ97023 |
| X₂₈₈₂ | M3 | 79 | 1069426 | C | T | [symbol] | CDZ97032 |
| X₂₈₈₃ | M3 | 79 | 1082233 | G | A | [symbol] | CDZ97037 |
| X₂₈₈₄ | M3 | 79 | 1118598 | C | T | [symbol] | CDZ97048 |
| X₂₈₈₅ | M3 | 79 | 1123288 | C | T | [symbol] | CDZ97049 |
| X₂₈₈₆ | M3 | 79 | 1140763 | G | A | [symbol] | CDZ97053 |
| X₂₈₈₇ | M3 | 79 | 1149291 | G | A | [symbol] | CDZ97056 |
| X₂₈₈₈ | M3 | 79 | 1211985 | G | A | [symbol] | CDZ97077 |
| X₂₈₈₉ | M3 | 79 | 1229129 | G | A | [symbol] | CDZ97086 |
| X₂₈₉₀ | M3 | 79 | 1231740 | C | T | [symbol] | CDZ97087 |
| X₂₈₉₁ | M3 | 79 | 1265502 | C | T | [symbol] | CDZ97100 |
| X₂₈₉₂ | M3 | 79 | 1279548 | G | A | [symbol] | CDZ97105 |
| X₂₈₉₃ | M3 | 79 | 1318034 | C | T | [symbol] | CDZ97117 |
| X₂₈₉₄ | M3 | 79 | 1336766 | C | T | [symbol] | CDZ97121 |
| X₂₈₉₅ | M3 | 79 | 1371491 | C | T | [symbol] | CDZ97129 |
| X₂₈₉₆ | M3 | 79 | 1394034 | G | A | [symbol] | CDZ97137 |
| X₂₈₉₇ | M3 | 79 | 1410287 | C | T | [symbol] | CDZ97145 |
| X₂₈₉₈ | M3 | 79 | 1410481 | G | A | [symbol] | CDZ97145 |
| X₂₈₉₉ | M3 | 79 | 1430954 | G | A | [symbol] | CDZ97152 |
| X₂₉₀₀ | M3 | 79 | 1540205 | C | T | [symbol] | CDZ97186 |
| X₂₉₀₁ | M3 | 79 | 1556423 | C | T | [symbol] | CDZ97191 |
| X₂₉₀₂ | M3 | 79 | 1624415 | G | A | [symbol] | CDZ97217 |
| X₂₉₀₃ | M3 | 79 | 1651212 | C | T | [symbol] | CDZ97224 |
| X₂₉₀₄ | M3 | 79 | 1674448 | G | A | [symbol] | CDZ97228 |
| X₂₉₀₅ | M3 | 79 | 1788096 | G | A | [symbol] | CDZ97262 |
| X₂₉₀₆ | M3 | 79 | 1791482 | G | A | [symbol] | CDZ97263 |
| X₂₉₀₇ | M3 | 79 | 1797660 | C | T | [symbol] | CDZ97265 |
| X₂₉₀₈ | M3 | 79 | 1797676 | G | A | [symbol] | CDZ97265 |
| X₂₉₀₉ | M3 | 79 | 1809211 | C | T | [symbol] | CDZ97268 |
| X₂₉₁₀ | M3 | 79 | 1842448 | G | A | [symbol] | CDZ97282 |
| X₂₉₁₁ | M3 | 79 | 1842834 | G | A | [symbol] | CDZ97282 |
| X₂₉₁₂ | M3 | 79 | 1850295 | G | A | [symbol] | CDZ97285 |
| X₂₉₁₃ | M3 | 79 | 1855037 | A | G | [symbol] | CDZ97285 |
| X₂₉₁₄ | M3 | 79 | 1859220 | A | G | [symbol] | CDZ97291 |
| X₂₉₁₅ | M3 | 79 | 1877329 | G | A | [symbol] | CDZ97295 |
| X₂₉₁₆ | M3 | 79 | 1930688 | G | A | [symbol] | CDZ97315 |
| X₂₉₁₇ | M3 | 79 | 1990424 | G | A | [symbol] | CDZ97334 |
| X₂₉₁₈ | M3 | 79 | 2013361 | C | T | [symbol] | CDZ97344 |
| X₂₉₁₉ | M3 | 79 | 2066016 | C | T | [symbol] | CDZ97366 |
| X₂₉₂₀ | M3 | 79 | 2115870 | G | A | [symbol] | CDZ97380 |
| X₂₉₂₁ | M3 | 79 | 2121041 | G | A | [symbol] | CDZ97380 |
| X₂₉₂₂ | M3 | 79 | 2152573 | C | T | [symbol] | CDZ97392 |
| X₂₉₂₃ | M3 | 79 | 2172085 | C | T | [symbol] | CDZ97397 |
| X₂₉₂₄ | M3 | 79 | 2179936 | G | A | [symbol] | CDZ97402 |
| X₂₉₂₅ | M3 | 79 | 2188410 | G | A | [symbol] | CDZ97405 |
| X₂₉₂₆ | M3 | 79 | 2268540 | C | T | [symbol] | CDZ97435 |
| X₂₉₂₇ | M3 | 79 | 2315869 | A | C | [symbol] | CDZ97452 |
| X₂₉₂₈ | M3 | 79 | 2323713 | C | T | [symbol] | CDZ97455 |
| X₂₉₂₉ | M3 | 79 | 2339881 | C | G | [symbol] | CDZ97460 |
| X₂₉₃₀ | M3 | 79 | 2340326 | G | A | [symbol] | CDZ97462 |
| X₂₉₃₁ | M3 | 85 | 421 | G | A | [symbol] | |
| X₂₉₃₂ | M3 | 85 | 425 | C | T | [symbol] | |
| X₂₉₃₃ | M3 | 85 | 462 | G | A | [symbol] | |
| X₂₉₃₄ | M3 | 85 | 463 | A | G | [symbol] | |
| X₂₉₃₅ | M3 | 85 | 467 | A | T | [symbol] | |
| X₂₉₃₆ | M3 | 85 | 512 | A | G | [symbol] | |
| X₂₉₃₇ | M3 | 85 | 516 | G | A | [symbol] | |
| X₂₉₃₈ | M3 | 85 | 521 | C | T | [symbol] | |
| X₂₉₃₉ | M3 | 85 | 522 | A | G | [symbol] | |
| X₂₉₄₀ | M3 | 86 | 230 | A | G | [symbol] | |
| X₂₉₄₁ | M3 | 88 | 119 | C | T | [symbol] | CDZ97464 |
| X₂₉₄₂ | M3 | 88 | 196 | G | A | [symbol] | CDZ97464 |
| X₂₉₄₃ | M3 | 88 | 898 | C | T | [symbol] | CDZ97464 |
| X₂₉₄₄ | M3 | 99 | 245 | G | A | [symbol] | |
| X₂₉₄₅ | M3 | 103 | 768 | C | T | [symbol] | |
| X₂₉₄₆ | M3 | 106 | 278 | A | G | [symbol] | |
| X₂₉₄₇ | M3 | 109 | 396 | T | C | [symbol] | |
| X₂₉₄₈ | M3 | 118 | 396 | C | T | [symbol] | CDZ97478 |
| X₂₉₄₉ | M3 | 118 | 870 | G | A | [symbol] | CDZ97478 |
| X₂₉₅₀ | M3 | 118 | 1491 | G | A | [symbol] | CDZ97478 |
| X₂₉₅₁ | M3 | 121 | 907 | T | C | [symbol] | |
| X₂₉₅₂ | M3 | 121 | 1402 | C | G | [symbol] | |
| X₂₉₅₃ | M3 | 124 | 69730 | G | A | [symbol] | CDZ97501 |
| X₂₉₅₄ | M3 | 124 | 72117 | G | A | [symbol] | CDZ97501 |
| X₂₉₅₅ | M3 | 124 | 72760 | G | A | [symbol] | CDZ97501 |
| X₂₉₅₆ | M3 | 124 | 80266 | C | T | [symbol] | CDZ97504 |
| X₂₉₅₇ | M3 | 124 | 94611 | G | A | [symbol] | CDZ97507 |
| X₂₉₅₈ | M3 | 124 | 156052 | G | A | [symbol] | CDZ97524 |
| X₂₉₅₉ | M3 | 124 | 194455 | C | T | [symbol] | CDZ97538 |
| X₂₉₆₀ | M3 | 124 | 222249 | C | T | [symbol] | CDZ97547 |
| X₂₉₆₁ | M3 | 125 | 35864 | T | C | [symbol] | CDZ97573 |
| X₂₉₆₂ | M3 | 129 | 299 | G | A | [symbol] | CDZ97577 |
| X₂₉₆₃ | M3 | 129 | 433 | G | A | [symbol] | CDZ97577 |
| X₂₉₆₄ | M3 | 129 | 856 | G | A | [symbol] | CDZ97577 |
| X₂₉₆₅ | M3 | 129 | 1013 | C | T | [symbol] | CDZ97577 |
| X₂₉₆₆ | M3 | 130 | 674 | C | T | [symbol] | CDZ97578 |
| X₂₉₆₇ | M3 | 141 | 5649 | C | T | [symbol] | CDZ97582 |
| X₂₉₆₈ | M3 | 141 | 10162 | G | A | [symbol] | CDZ97582 |
| X₂₉₆₉ | M3 | 141 | 15764 | C | T | [symbol] | CDZ97587 |
| X₂₉₇₀ | M3 | 141 | 17894 | G | A | [symbol] | CDZ97587 |
| X₂₉₇₁ | M3 | 141 | 29260 | G | A | [symbol] | CDZ97594 |
| X₂₉₇₂ | M3 | 149 | 2074 | T | G | [symbol] | CDZ97598 |
| X₂₉₇₃ | M3 | 149 | 2453 | C | T | [symbol] | CDZ97598 |
| X₂₉₇₄ | M3 | 159 | 106 | A | C | [symbol] | |
| X₂₉₇₅ | M3 | 162 | 70025 | C | T | [symbol] | CDZ97625 |
| X₂₉₇₆ | M3 | 162 | 70405 | A | G | [symbol] | CDZ97625 |
| X₂₉₇₇ | M3 | 162 | 93443 | G | A | [symbol] | CDZ97631 |
| X₂₉₇₈ | M3 | 162 | 96194 | G | A | [symbol] | CDZ97631 |
| X₂₉₇₉ | M3 | 162 | 223407 | C | T | [symbol] | CDZ97674 |
| X₂₉₈₀ | M3 | 162 | 227242 | G | A | [symbol] | CDZ97674 |
| X₂₉₈₁ | M3 | 162 | 230526 | C | T | [symbol] | CDZ97676 |
| X₂₉₈₂ | M3 | 162 | 291566 | G | A | [symbol] | CDZ97695 |
| X₂₉₈₃ | M3 | 162 | 308369 | T | C | [symbol] | CDZ97702 |
| X₂₉₈₄ | M3 | 162 | 321070 | C | T | [symbol] | CDZ97703 |
| X₂₉₈₅ | M3 | 162 | 321073 | A | G | [symbol] | CDZ97703 |
| X₂₉₈₆ | M3 | 162 | 321460 | C | G | [symbol] | CDZ97703 |
| X₂₉₈₇ | M3 | 162 | 321463 | A | C | [symbol] | CDZ97703 |
| X₂₉₈₈ | M3 | 162 | 322591 | G | A | [symbol] | CDZ97703 |
| X₂₉₈₉ | M3 | 162 | 323448 | T | G | [symbol] | CDZ97703 |
| X₂₉₉₀ | M3 | 162 | 323451 | T | G | [symbol] | CDZ97703 |
| X₂₉₉₁ | M3 | 162 | 323480 | T | C | [symbol] | CDZ97703 |
| X₂₉₉₂ | M3 | 162 | 323735 | A | G | [symbol] | CDZ97703 |
| X₂₉₉₃ | M3 | 162 | 324114 | T | C | [symbol] | CDZ97703 |
| X₂₉₉₄ | M3 | 162 | 324139 | C | T | [symbol] | CDZ97703 |
| X₂₉₉₅ | M3 | 162 | 324568 | A | G | [symbol] | CDZ97703 |
| X₂₉₉₆ | M3 | 162 | 341717 | G | A | [symbol] | CDZ97707 |
| X₂₉₉₇ | M3 | 162 | 351016 | C | T | [symbol] | CDZ97711 |
| X₂₉₉₈ | M3 | 162 | 360320 | G | A | [symbol] | CDZ97714 |
| X₂₉₉₉ | M3 | 162 | 423180 | C | T | [symbol] | CDZ97740 |
| X₃₀₀₀ | M3 | 162 | 424391 | C | T | [symbol] | CDZ97740 |
| X₃₀₀₁ | M3 | 162 | 444123 | T | C | [symbol] | CDZ97748 |
| X₃₀₀₂ | M3 | 162 | 456498 | C | T | [symbol] | CDZ97752 |
| X₃₀₀₃ | M3 | 162 | 467207 | C | T | [symbol] | CDZ97755 |
| X₃₀₀₄ | M3 | 162 | 476928 | C | T | [symbol] | CDZ97760 |
| X₃₀₀₅ | M3 | 162 | 491906 | G | A | [symbol] | CDZ97764 |
| X₃₀₀₆ | M3 | 162 | 492078 | G | A | [symbol] | CDZ97764 |
| X₃₀₀₇ | M3 | 162 | 492101 | G | A | [symbol] | CDZ97764 |
| X₃₀₀₈ | M3 | 162 | 501886 | C | T | [symbol] | CDZ97769 |
| X₃₀₀₉ | M3 | 162 | 632311 | G | A | [symbol] | CDZ97814 |
| X₃₀₁₀ | M3 | 162 | 633321 | G | A | [symbol] | CDZ97814 |
| X₃₀₁₁ | M3 | 162 | 633633 | G | A | [symbol] | CDZ97814 |
| X₃₀₁₂ | M3 | 162 | 640005 | C | T | [symbol] | CDZ97815 |
| X₃₀₁₃ | M3 | 162 | 645194 | G | A | [symbol] | CDZ97815 |
| X₃₀₁₄ | M3 | 162 | 674192 | C | T | [symbol] | CDZ97824 |
| X₃₀₁₅ | M3 | 162 | 738675 | G | A | [symbol] | CDZ97842 |
| X₃₀₁₆ | M3 | 162 | 738686 | C | T | [symbol] | CDZ97842 |
| X₃₀₁₇ | M3 | 162 | 792386 | G | A | [symbol] | CDZ97863 |
| X₃₀₁₈ | M3 | 162 | 801198 | C | T | [symbol] | CDZ97864 |
| X₃₀₁₉ | M3 | 162 | 808830 | G | A | [symbol] | CDZ97869 |
| X₃₀₂₀ | M3 | 162 | 848369 | G | A | [symbol] | CDZ97884 |
| X₃₀₂₁ | M3 | 162 | 871911 | C | T | [symbol] | CDZ97890 |
| X₃₀₂₂ | M3 | 162 | 885927 | C | T | [symbol] | CDZ97896 |
| X₃₀₂₃ | M3 | 162 | 966019 | C | T | [symbol] | CDZ97923 |
| X₃₀₂₄ | M3 | 162 | 972973 | G | A | [symbol] | CDZ97925 |
| X₃₀₂₅ | M3 | 175 | 385 | A | C | [symbol] | |
| X₃₀₂₆ | M3 | 189 | 32 | G | A | [symbol] | CDZ97960 |
| X₃₀₂₇ | M3 | 189 | 108326 | C | T | [symbol] | CDZ97991 |
| X₃₀₂₈ | M3 | 189 | 126573 | G | A | [symbol] | CDZ97996 |
| X₃₀₂₉ | M3 | 189 | 127854 | C | T | [symbol] | CDZ97998 |
| X₃₀₃₀ | M3 | 189 | 174918 | G | A | [symbol] | CDZ98015 |
| X₃₀₃₁ | M3 | 189 | 194647 | A | G | [symbol] | CDZ98020 |
| X₃₀₃₂ | M3 | 189 | 194665 | A | G | [symbol] | CDZ98020 |
| X₃₀₃₃ | M3 | 189 | 298487 | C | T | [symbol] | CDZ98056 |
| X₃₀₃₄ | M3 | 189 | 323680 | C | T | [symbol] | CDZ98063 |
| X₃₀₃₅ | M3 | 189 | 371247 | G | A | [symbol] | CDZ98078 |
| X₃₀₃₆ | M3 | 189 | 394551 | C | T | [symbol] | CDZ98085 |
| X₃₀₃₇ | M3 | 189 | 452298 | G | A | [symbol] | CDZ98106 |
| X₃₀₃₈ | M3 | 191 | 342 | A | G | [symbol] | CDZ98107 |
| X₃₀₃₉ | M3 | 191 | 347 | C | T | [symbol] | CDZ98107 |
| X₃₀₄₀ | M3 | 191 | 427 | C | T | [symbol] | CDZ98107 |
| X₃₀₄₁ | M3 | 191 | 451 | C | T | [symbol] | CDZ98107 |
| X₃₀₄₂ | M3 | 191 | 487 | A | G | [symbol] | CDZ98107 |
| X₃₀₄₃ | M3 | 197 | 624 | C | T | [symbol] | CDZ98111 |
| X₃₀₄₄ | M3 | 198 | 202 | C | A | [symbol] | |
| X₃₀₄₅ | M3 | 198 | 203 | G | A | [symbol] | |
| X₃₀₄₆ | M3 | 198 | 238 | C | T | [symbol] | |
| X₃₀₄₇ | M3 | 198 | 265 | C | T | [symbol] | |
| X₃₀₄₈ | M3 | 199 | 688 | C | T | [symbol] | |
| X₃₀₄₉ | M3 | 199 | 911 | C | T | [symbol] | |
| X₃₀₅₀ | M3 | 199 | 971 | C | T | [symbol] | |
| X₃₀₅₁ | M3 | 199 | 1218 | C | T | [symbol] | |
| X₃₀₅₂ | M3 | 202 | 171 | C | T | [symbol] | |
| X₃₀₅₃ | M3 | 205 | 360 | C | T | [symbol] | CDZ98112 |
| X₃₀₅₄ | M3 | 205 | 981 | C | T | [symbol] | CDZ98112 |
| X₃₀₅₅ | M3 | 208 | 117 | G | A | [symbol] | |
| X₃₀₅₆ | M3 | 208 | 476 | G | A | [symbol] | |
| X₃₀₅₇ | M3 | 208 | 616 | G | A | [symbol] | |
| X₃₀₅₈ | M3 | 208 | 935 | G | A | [symbol] | |
| X₃₀₅₉ | M3 | 209 | 312 | C | T | [symbol] | CDZ98113 |
| X₃₀₆₀ | M3 | 223 | 93 | G | A | [symbol] | CDZ98118 |
| X₃₀₆₁ | M3 | 223 | 102 | A | G | [symbol] | CDZ98118 |
| X₃₀₆₂ | M3 | 223 | 133 | A | G | [symbol] | CDZ98118 |
| X₃₀₆₃ | M3 | 223 | 150 | T | C | [symbol] | CDZ98118 |
| X₃₀₆₄ | M3 | 223 | 155 | A | C | [symbol] | CDZ98118 |
| X₃₀₆₅ | M3 | 223 | 158 | T | C | [symbol] | CDZ98118 |
| X₃₀₆₆ | M3 | 223 | 160 | C | T | [symbol] | CDZ98118 |
| X₃₀₆₇ | M3 | 223 | 175 | G | A | [symbol] | CDZ98118 |
| X₃₀₆₈ | M3 | 223 | 177 | A | G | [symbol] | CDZ98118 |
| X₃₀₆₉ | M3 | 223 | 188 | A | G | [symbol] | CDZ98118 |
| X₃₀₇₀ | M3 | 223 | 192 | C | A | [symbol] | CDZ98118 |
| X₃₀₇₁ | M3 | 223 | 194 | T | C | [symbol] | CDZ98118 |
| X₃₀₇₂ | M3 | 223 | 195 | T | G | [symbol] | CDZ98118 |
| X₃₀₇₃ | M3 | 223 | 197 | T | C | [symbol] | CDZ98118 |
| X₃₀₇₄ | M3 | 223 | 202 | G | C | [symbol] | CDZ98118 |
| X₃₀₇₅ | M3 | 223 | 205 | C | T | [symbol] | CDZ98118 |
| X₃₀₇₆ | M3 | 223 | 291 | C | A | [symbol] | CDZ98118 |
| X₃₀₇₇ | M3 | 223 | 360 | C | T | [symbol] | CDZ98118 |
| X₃₀₇₈ | M3 | 225 | 317 | C | T | [symbol] | |
| X₃₀₇₉ | M3 | 227 | 202 | G | A | [symbol] | |
| X₃₀₈₀ | M3 | 229 | 225 | G | C | [symbol] | |
| X₃₀₈₁ | M3 | 232 | 340 | G | A | [symbol] | CDZ98119 |
| X₃₀₈₂ | M3 | 232 | 1379 | C | T | [symbol] | CDZ98119 |
| X₃₀₈₃ | M3 | 232 | 1521 | C | T | [symbol] | CDZ98119 |
| X₃₀₈₄ | M3 | 232 | 1662 | G | A | [symbol] | CDZ98119 |
| X₃₀₈₅ | M3 | 232 | 1761 | C | T | [symbol] | CDZ98119 |
| X₃₀₈₆ | M3 | 232 | 2027 | C | T | [symbol] | CDZ98119 |
| X₃₀₈₇ | M3 | 232 | 3060 | C | T | [symbol] | CDZ98120 |
| X₃₀₈₈ | M3 | 232 | 3284 | C | T | [symbol] | CDZ98119 |
| X₃₀₈₉ | M3 | 232 | 4437 | G | A | [symbol] | CDZ98121 |
| X₃₀₉₀ | M3 | 232 | 4561 | C | T | [symbol] | CDZ98120 |
| X₃₀₉₁ | M3 | 242 | 7334 | C | T | [symbol] | CDZ98125 |
| X₃₀₉₂ | M3 | 242 | 41710 | G | A | [symbol] | CDZ98131 |
| X₃₀₉₃ | M3 | 242 | 50262 | G | A | [symbol] | CDZ98135 |
| X₃₀₉₄ | M3 | 242 | 107587 | G | A | [symbol] | CDZ98153 |
| X₃₀₉₅ | M3 | 242 | 125424 | C | T | [symbol] | CDZ98159 |
| X₃₀₉₆ | M3 | 242 | 150461 | C | T | [symbol] | CDZ98164 |
| X₃₀₉₇ | M3 | 242 | 168689 | G | A | [symbol] | CDZ98172 |
| X₃₀₉₈ | M3 | 242 | 189498 | G | A | [symbol] | CDZ98176 |
| X₃₀₉₉ | M3 | 242 | 194983 | C | T | [symbol] | CDZ98179 |
| X₃₁₀₀ | M3 | 242 | 213986 | G | A | [symbol] | CDZ98185 |
| X₃₁₀₁ | M3 | 242 | 219778 | C | T | [symbol] | CDZ98187 |
| X₃₁₀₂ | M3 | 242 | 220015 | G | A | [symbol] | CDZ98187 |
| X₃₁₀₃ | M3 | 242 | 227271 | C | T | [symbol] | CDZ98191 |
| X₃₁₀₄ | M3 | 242 | 233088 | G | A | [symbol] | CDZ98193 |
| X₃₁₀₅ | M3 | 242 | 233141 | G | A | [symbol] | CDZ98193 |
| X₃₁₀₆ | M3 | 242 | 310353 | G | A | [symbol] | CDZ98216 |
| X₃₁₀₇ | M3 | 242 | 342570 | C | T | [symbol] | CDZ98229 |
| X₃₁₀₈ | M3 | 242 | 363677 | G | A | [symbol] | CDZ98233 |
| X₃₁₀₉ | M3 | 242 | 376713 | G | A | [symbol] | CDZ98238 |
| X₃₁₁₀ | M3 | 242 | 404132 | G | A | [symbol] | CDZ98248 |
| X₃₁₁₁ | M3 | 242 | 412676 | G | A | [symbol] | CDZ98249 |
| X₃₁₁₂ | M3 | 242 | 437941 | G | A | [symbol] | CDZ98258 |
| X₃₁₁₃ | M3 | 242 | 472562 | C | T | [symbol] | CDZ98266 |
| X₃₁₁₄ | M3 | 242 | 485936 | G | A | [symbol] | CDZ98270 |
| X₃₁₁₅ | M3 | 242 | 492389 | G | A | [symbol] | CDZ98274 |
| X₃₁₁₆ | M3 | 242 | 514780 | C | T | [symbol] | CDZ98282 |
| X₃₁₁₇ | M3 | 242 | 542663 | G | A | [symbol] | CDZ98290 |
| X₃₁₁₈ | M3 | 242 | 559999 | G | A | [symbol] | CDZ98297 |
| X₃₁₁₉ | M3 | 242 | 570416 | G | A | [symbol] | CDZ98299 |
| X₃₁₂₀ | M3 | 242 | 577367 | G | A | [symbol] | CDZ98302 |
| X₃₁₂₁ | M3 | 242 | 602456 | G | A | [symbol] | CDZ98308 |
| X₃₁₂₂ | M3 | 242 | 602531 | G | A | [symbol] | CDZ98308 |
| X₃₁₂₃ | M3 | 242 | 604704 | C | T | [symbol] | CDZ98309 |
| X₃₁₂₄ | M3 | 242 | 641669 | G | A | [symbol] | CDZ98323 |
| X₃₁₂₅ | M3 | 242 | 649715 | G | A | [symbol] | CDZ98326 |
| X₃₁₂₆ | M3 | 246 | 329 | C | G | [symbol] | CDZ98328 |
| X₃₁₂₇ | M3 | 246 | 331 | T | C | [symbol] | CDZ98328 |
| X₃₁₂₈ | M3 | 249 | 2757 | C | T | [symbol] | CED84929 |
| X₃₁₂₉ | M3 | 249 | 2763 | A | T | [symbol] | CED84929 |
| X₃₁₃₀ | M3 | 249 | 5307 | C | T | [symbol] | CED84929 |
| X₃₁₃₁ | M3 | 249 | 72016 | C | T | [symbol] | CED84950 |
| X₃₁₃₂ | M3 | 249 | 123789 | C | T | [symbol] | CED84972 |
| X₃₁₃₃ | M3 | 249 | 157550 | C | T | [symbol] | CED84983 |
| X₃₁₃₄ | M3 | 249 | 191304 | C | T | [symbol] | CED84988 |
| X₃₁₃₅ | M3 | 249 | 253494 | C | T | [symbol] | CED85008 |
| X₃₁₃₆ | M3 | 249 | 272075 | C | T | [symbol] | CED85013 |
| X₃₁₃₇ | M3 | 249 | 289154 | C | T | [symbol] | CED85018 |
| X₃₁₃₈ | M3 | 249 | 294161 | G | A | [symbol] | CED85018 |
| X₃₁₃₉ | M3 | 249 | 294853 | C | T | [symbol] | CED85019 |
| X₃₁₄₀ | M3 | 249 | 305975 | G | A | [symbol] | CED85023 |
| X₃₁₄₁ | M3 | 249 | 326271 | C | T | [symbol] | CED85029 |
| X₃₁₄₂ | M3 | 249 | 342990 | G | A | [symbol] | CED85036 |
| X₃₁₄₃ | M3 | 249 | 445429 | G | A | [symbol] | CED85075 |
| X₃₁₄₄ | M3 | 249 | 469645 | G | A | [symbol] | CED85087 |
| X₃₁₄₅ | M3 | 249 | 476060 | A | G | [symbol] | CED85088 |
| X₃₁₄₆ | M3 | 249 | 485782 | C | T | [symbol] | CED85089 |
| X₃₁₄₇ | M3 | 249 | 487588 | G | A | [symbol] | CED85090 |
| X₃₁₄₈ | M3 | 249 | 500195 | C | T | [symbol] | CED85093 |
| X₃₁₄₉ | M3 | 249 | 501310 | C | T | [symbol] | CED85093 |
| X₃₁₅₀ | M3 | 249 | 501510 | T | C | [symbol] | CED85093 |
| X₃₁₅₁ | M3 | 249 | 532943 | A | G | [symbol] | CED85099 |
| X₃₁₅₂ | M3 | 249 | 565916 | C | T | [symbol] | CED85113 |
| X₃₁₅₃ | M3 | 249 | 571922 | C | T | [symbol] | CED85115 |
| X₃₁₅₄ | M3 | 249 | 576070 | G | A | [symbol] | CED85118 |
| X₃₁₅₅ | M3 | 249 | 598579 | C | T | [symbol] | CED85125 |
| X₃₁₅₆ | M3 | 249 | 634793 | A | G | [symbol] | CED85133 |
| X₃₁₅₇ | M3 | 249 | 637944 | G | A | [symbol] | CED85135 |
| X₃₁₅₈ | M3 | 249 | 660408 | G | A | [symbol] | CED85144 |
| X₃₁₅₉ | M3 | 249 | 854220 | G | A | [symbol] | CED85201 |
| X₃₁₆₀ | M3 | 249 | 889853 | G | A | [symbol] | CED85211 |
| X₃₁₆₁ | M3 | 249 | 911274 | C | T | [symbol] | CED85218 |
| X₃₁₆₂ | M3 | 249 | 932606 | C | T | [symbol] | CED85223 |
| X₃₁₆₃ | M3 | 249 | 934305 | G | A | [symbol] | CED85225 |
| X₃₁₆₄ | M3 | 249 | 935435 | C | T | [symbol] | CED85225 |
| X₃₁₆₅ | M3 | 249 | 945434 | C | T | [symbol] | CED85227 |
| X₃₁₆₆ | M3 | 249 | 945727 | C | T | [symbol] | CED85227 |
| X₃₁₆₇ | M3 | 249 | 969333 | G | T | [symbol] | CED85238 |
| X₃₁₆₈ | M3 | 249 | 972975 | G | A | [symbol] | CED85240 |
| X₃₁₆₉ | M3 | 249 | 977170 | C | T | [symbol] | CED85242 |
| X₃₁₇₀ | M3 | 249 | 989520 | C | T | [symbol] | CED85243 |
| X₃₁₇₁ | M3 | 249 | 1014098 | C | T | [symbol] | CED85252 |
| X₃₁₇₂ | M3 | 249 | 1088841 | T | C | [symbol] | CED85274 |
| X₃₁₇₃ | M3 | 249 | 1116964 | G | A | [symbol] | CED85283 |
| X₃₁₇₄ | M3 | 249 | 1144309 | G | A | [symbol] | CED85291 |
| X₃₁₇₅ | M3 | 249 | 1155955 | C | T | [symbol] | CED85297 |
| X₃₁₇₆ | M3 | 249 | 1176977 | G | A | [symbol] | CED85303 |
| X₃₁₇₇ | M3 | 249 | 1195367 | G | A | [symbol] | CED85307 |
| X₃₁₇₈ | M3 | 249 | 1209489 | C | T | [symbol] | CED85313 |
| X₃₁₇₉ | M3 | 249 | 1230068 | C | T | [symbol] | CED85319 |
| X₃₁₈₀ | M3 | 249 | 1286733 | G | A | [symbol] | CED85336 |
| X₃₁₈₁ | M3 | 249 | 1299472 | C | T | [symbol] | CED85343 |
| X₃₁₈₂ | M3 | 249 | 1310059 | C | T | [symbol] | CED85346 |
| X₃₁₈₃ | M3 | 249 | 1473930 | C | T | [symbol] | CED85404 |
| X₃₁₈₄ | M3 | 249 | 1473937 | G | A | [symbol] | CED85404 |
| X₃₁₈₅ | M3 | 249 | 1486325 | G | A | [symbol] | CED85409 |
| X₃₁₈₆ | M3 | 249 | 1498029 | T | C | [symbol] | CED85415 |
| X₃₁₈₇ | M3 | 249 | 1498884 | T | C | [symbol] | CED85414 |
| X₃₁₈₈ | M3 | 249 | 1499008 | A | G | [symbol] | CED85415 |
| X₃₁₈₉ | M3 | 249 | 1502631 | G | T | [symbol] | CED85416 |
| X₃₁₉₀ | M3 | 249 | 1502641 | T | A | [symbol] | CED85416 |
| X₃₁₉₁ | M3 | 249 | 1502819 | T | G | [symbol] | CED85416 |
| X₃₁₉₂ | M3 | 249 | 1505138 | G | A | [symbol] | CED85416 |
| X₃₁₉₃ | M3 | 249 | 1505634 | T | G | [symbol] | CED85416 |
| X₃₁₉₄ | M3 | 249 | 1519431 | A | G | [symbol] | CED85419 |
| X₃₁₉₅ | M3 | 249 | 1523260 | C | T | [symbol] | CED85419 |
| X₃₁₉₆ | M3 | 249 | 1599907 | C | T | [symbol] | CED85444 |
| X₃₁₉₇ | M3 | 249 | 1619036 | G | A | [symbol] | CED85450 |
| X₃₁₉₈ | M3 | 249 | 1623151 | G | A | [symbol] | CED85451 |
| X₃₁₉₉ | M3 | 249 | 1653615 | T | G | [symbol] | CED85457 |
| X₃₂₀₀ | M3 | 249 | 1653962 | C | T | [symbol] | CED85458 |
| X₃₂₀₁ | M3 | 249 | 1666185 | C | T | [symbol] | CED85460 |
| X₃₂₀₂ | M3 | 249 | 1726777 | C | T | [symbol] | CED85474 |
| X₃₂₀₃ | M3 | 249 | 1760715 | C | T | [symbol] | CED85489 |
| X₃₂₀₄ | M3 | 249 | 1769706 | A | G | [symbol] | CED85490 |
| X₃₂₀₅ | M3 | 249 | 1780551 | A | G | [symbol] | CED85493 |
| X₃₂₀₆ | M3 | 249 | 1780569 | A | G | [symbol] | CED85493 |
| X₃₂₀₇ | M3 | 249 | 1801965 | G | A | [symbol] | CED85501 |
| X₃₂₀₈ | M3 | 249 | 1803575 | C | T | [symbol] | CED85502 |
| X₃₂₀₉ | M3 | 249 | 1804835 | G | A | [symbol] | CED85502 |
| X₃₂₁₀ | M3 | 249 | 1880762 | G | A | [symbol] | CED85525 |
| X₃₂₁₁ | M3 | 249 | 1944026 | G | A | [symbol] | CED85545 |
| X₃₂₁₂ | M3 | 249 | 1981230 | G | A | [symbol] | CED85557 |
| X₃₂₁₃ | M3 | 249 | 1988505 | C | T | [symbol] | CED85560 |
| X₃₂₁₄ | M3 | 249 | 2015272 | G | A | [symbol] | CED85567 |
| X₃₂₁₅ | M3 | 249 | 2027477 | G | A | [symbol] | CED85573 |
| X₃₂₁₆ | M3 | 249 | 2047401 | C | T | [symbol] | CED85581 |
| X₃₂₁₇ | M3 | 249 | 2110956 | C | T | [symbol] | CED85603 |
| X₃₂₁₈ | M3 | 249 | 2163304 | G | A | [symbol] | CED85618 |
| X₃₂₁₉ | M3 | 249 | 2167664 | G | A | [symbol] | CED85620 |
| X₃₂₂₀ | M3 | 249 | 2178506 | G | A | [symbol] | CED85622 |
| X₃₂₂₁ | M3 | 249 | 2183695 | G | A | [symbol] | CED85623 |
| X₃₂₂₂ | M3 | 249 | 2198205 | G | A | [symbol] | CED85628 |
| X₃₂₂₃ | M3 | 249 | 2223626 | G | A | [symbol] | CED85633 |
| X₃₂₂₄ | M3 | 249 | 2255547 | G | C | [symbol] | CED85640 |
| X₃₂₂₅ | M3 | 25 | 1089 | T | C | [symbol] | |
| X₃₂₂₆ | M3 | 252 | 512 | C | T | [symbol] | |
| X₃₂₂₇ | M3 | 252 | 524 | C | T | [symbol] | |
| X₃₂₂₈ | M3 | 258 | 836 | A | G | [symbol] | |
| X₃₂₂₉ | M3 | 258 | 1139 | A | T | [symbol] | |
| X₃₂₃₀ | M3 | 260 | 156 | G | A | [symbol] | |
| X₃₂₃₁ | M3 | 262 | 4049 | G | A | [symbol] | CDZ98330 |
| X₃₂₃₂ | M3 | 262 | 12289 | C | T | [symbol] | CDZ98333 |
| X₃₂₃₃ | M3 | 262 | 30347 | G | A | [symbol] | CDZ98338 |
| X₃₂₃₄ | M3 | 262 | 30359 | G | A | [symbol] | CDZ98338 |
| X₃₂₃₅ | M3 | 262 | 81226 | G | A | [symbol] | CDZ98358 |
| X₃₂₃₆ | M3 | 262 | 108865 | C | T | [symbol] | CDZ98369 |
| X₃₂₃₇ | M3 | 262 | 143387 | C | T | [symbol] | CDZ98375 |
| X₃₂₃₈ | M3 | 262 | 148151 | C | T | [symbol] | CDZ98375 |
| X₃₂₃₉ | M3 | 262 | 153091 | G | A | [symbol] | CDZ98377 |
| X₃₂₄₀ | M3 | 262 | 164438 | G | A | [symbol] | CDZ98383 |
| X₃₂₄₁ | M3 | 262 | 201531 | G | A | [symbol] | CDZ98396 |
| X₃₂₄₂ | M3 | 262 | 221937 | C | T | [symbol] | CDZ98403 |
| X₃₂₄₃ | M3 | 262 | 309128 | C | T | [symbol] | CDZ98434 |
| X₃₂₄₄ | M3 | 262 | 319302 | G | A | [symbol] | CDZ98436 |
| X₃₂₄₅ | M3 | 262 | 351354 | G | A | [symbol] | CDZ98450 |
| X₃₂₄₆ | M3 | 262 | 380519 | C | T | [symbol] | CDZ98457 |
| X₃₂₄₇ | M3 | 262 | 383215 | C | T | [symbol] | CDZ98459 |
| X₃₂₄₈ | M3 | 262 | 397539 | G | A | [symbol] | CDZ98463 |
| X₃₂₄₉ | M3 | 262 | 414403 | C | T | [symbol] | CDZ98467 |
| X₃₂₅₀ | M3 | 262 | 420102 | G | A | [symbol] | CDZ98468 |
| X₃₂₅₁ | M3 | 262 | 444331 | G | A | [symbol] | CDZ98477 |
| X₃₂₅₂ | M3 | 262 | 446956 | C | T | [symbol] | CDZ98477 |
| X₂₂₅₃ | M3 | 262 | 451669 | C | T | [symbol] | CDZ98478 |
| X₃₂₅₄ | M3 | 262 | 531136 | G | A | [symbol] | CDZ98510 |
| X₃₂₅₅ | M3 | 262 | 539452 | A | G | [symbol] | CDZ98514 |
| X₃₂₅₆ | M3 | 262 | 557280 | G | A | [symbol] | CDZ98518 |
| X₃₂₅₇ | M3 | 262 | 560713 | G | A | [symbol] | CDZ98519 |
| X₃₂₅₈ | M3 | 262 | 562795 | C | T | [symbol] | CDZ98521 |
| X₃₂₅₉ | M3 | 262 | 567591 | G | A | [symbol] | CDZ98524 |
| X₃₂₆₀ | M3 | 262 | 567801 | G | A | [symbol] | CDZ98524 |
| X₃₂₆₁ | M3 | 262 | 573667 | C | T | [symbol] | CDZ98526 |
| X₃₂₆₂ | M3 | 262 | 607661 | G | A | [symbol] | CDZ98533 |
| X₃₂₆₃ | M3 | 262 | 618198 | G | A | [symbol] | CDZ98538 |
| X₃₂₆₄ | M3 | 262 | 627573 | T | C | [symbol] | CDZ98542 |
| X₃₂₆₅ | M3 | 262 | 638804 | G | C | [symbol] | CDZ98544 |
| X₃₂₆₆ | M3 | 262 | 638986 | C | G | [symbol] | CDZ98544 |
| X₃₂₆₇ | M3 | 262 | 639006 | C | T | [symbol] | CDZ98544 |
| X₃₂₆₈ | M3 | 262 | 639869 | G | C | [symbol] | CDZ98544 |
| X₃₂₆₉ | M3 | 262 | 639962 | A | G | [symbol] | CDZ98544 |
| X₃₂₇₀ | M3 | 262 | 640066 | A | G | [symbol] | CDZ98544 |
| X₃₂₇₁ | M3 | 262 | 653920 | A | G | [symbol] | CDZ98545 |
| X₃₂₇₂ | M3 | 262 | 655754 | A | T | [symbol] | CDZ98545 |
| X₃₂₇₃ | M3 | 262 | 655757 | T | C | [symbol] | CDZ98545 |
| X₃₂₇₄ | M3 | 262 | 656031 | C | A | [symbol] | CDZ98545 |
| X₃₂₇₅ | M3 | 262 | 656323 | C | G | [symbol] | CDZ98545 |
| X₃₂₇₆ | M3 | 262 | 668764 | C | T | [symbol] | CDZ98545 |
| X₃₂₇₇ | M3 | 262 | 691577 | C | T | [symbol] | CDZ98551 |
| X₃₂₇₈ | M3 | 262 | 714374 | A | C | [symbol] | CDZ98557 |
| X₃₂₇₉ | M3 | 262 | 714377 | A | G | [symbol] | CDZ98557 |
| X₃₂₈₀ | M3 | 262 | 771330 | G | A | [symbol] | CDZ98578 |
| X₃₂₈₁ | M3 | 262 | 801626 | G | A | [symbol] | CDZ98589 |
| X₃₂₈₂ | M3 | 262 | 822734 | G | A | [symbol] | CDZ98597 |
| X₃₂₈₃ | M3 | 262 | 835874 | C | T | [symbol] | CDZ98601 |
| X₃₂₈₄ | M3 | 262 | 865999 | G | A | [symbol] | CDZ98613 |
| X₃₂₈₅ | M3 | 262 | 971713 | G | A | [symbol] | CDZ98649 |
| X₃₂₈₆ | M3 | 262 | 1016286 | C | T | [symbol] | CDZ98661 |
| X₃₂₈₇ | M3 | 262 | 1044256 | G | A | [symbol] | CDZ98669 |
| X₃₂₈₈ | M3 | 262 | 1051389 | C | T | [symbol] | CDZ98670 |
| X₃₂₈₉ | M3 | 262 | 1060326 | C | T | [symbol] | CDZ98672 |
| X₃₂₉₀ | M3 | 262 | 1107286 | C | T | [symbol] | CDZ98689 |
| X₃₂₉₁ | M3 | 262 | 1140198 | C | T | [symbol] | CDZ98701 |
| X₃₂₉₂ | M3 | 262 | 1204782 | G | A | [symbol] | CDZ98722 |
| X₃₂₉₃ | M3 | 262 | 1209662 | G | A | [symbol] | CDZ98723 |
| X₃₂₉₄ | M3 | 262 | 1226072 | C | T | [symbol] | CDZ98730 |
| X₃₂₉₅ | M3 | 262 | 1233054 | G | A | [symbol] | CDZ98733 |
| X₃₂₉₆ | M3 | 262 | 1237021 | C | T | [symbol] | CDZ98735 |
| X₃₂₉₇ | M3 | 262 | 1277604 | G | A | [symbol] | CDZ98747 |
| X₃₂₉₈ | M3 | 262 | 1305361 | G | A | [symbol] | CDZ98753 |
| X₃₂₉₉ | M3 | 262 | 1329643 | C | T | [symbol] | CDZ98760 |
| X₃₃₀₀ | M3 | 262 | 1332176 | C | T | [symbol] | CDZ98761 |
| X₃₃₀₁ | M3 | 262 | 1349561 | G | A | [symbol] | CDZ98766 |
| X₃₃₀₂ | M3 | 262 | 1351116 | A | G | [symbol] | CDZ98766 |
| X₃₃₀₃ | M3 | 262 | 1415479 | C | T | [symbol] | CDZ98789 |
| X₃₃₀₄ | M3 | 262 | 1416604 | C | T | [symbol] | CDZ98788 |
| X₃₃₀₅ | M3 | 262 | 1424293 | G | A | [symbol] | CDZ98791 |
| X₃₃₀₆ | M3 | 262 | 1455677 | G | A | [symbol] | CDZ98804 |
| X₃₃₀₇ | M3 | 262 | 1458863 | C | T | [symbol] | CDZ98807 |
| X₃₃₀₈ | M3 | 262 | 1549966 | C | G | [symbol] | CDZ98834 |
| X₃₃₀₉ | M3 | 262 | 1550311 | C | T | [symbol] | CDZ98836 |
| X₃₃₁₀ | M3 | 262 | 1550456 | C | T | [symbol] | CDZ98837 |
| X₃₃₁₁ | M3 | 262 | 1558074 | G | A | [symbol] | CDZ98840 |
| X₃₃₁₂ | M3 | 262 | 1615762 | G | A | [symbol] | CDZ98855 |
| X₃₃₁₃ | M3 | 262 | 1694625 | C | T | [symbol] | CDZ98878 |
| X₃₃₁₄ | M3 | 262 | 1706076 | G | A | [symbol] | CDZ98885 |
| X₃₃₁₅ | M3 | 262 | 1709112 | G | A | [symbol] | CDZ98884 |
| X₃₃₁₆ | M3 | 262 | 1711527 | G | A | [symbol] | CDZ98886 |
| X₃₃₁₇ | M3 | 262 | 1716385 | G | A | [symbol] | CDZ98887 |
| X₃₃₁₈ | M3 | 262 | 1717611 | C | T | [symbol] | CDZ98888 |
| X₃₃₁₉ | M3 | 266 | 85 | C | T | [symbol] | |
| X₃₃₂₀ | M3 | 266 | 252 | C | T | [symbol] | |
| X₃₃₂₁ | M3 | 267 | 133 | T | C | [symbol] | |
| X₃₃₂₂ | M3 | 267 | 134 | C | T | [symbol] | |
| X₃₃₂₃ | M4 | 22 | 1643 | C | T | [symbol] | CED80056 |
| X₃₃₂₄ | M4 | 22 | 2524 | G | A | [symbol] | CED80056 |
| X₃₃₂₅ | M4 | 28 | 236 | G | A | [symbol] | CDZ96150 |
| X₃₃₂₆ | M4 | 28 | 377 | G | A | [symbol] | CDZ96150 |
| X₃₃₂₇ | M4 | 28 | 1506 | C | T | [symbol] | CDZ96150 |
| X₃₃₂₈ | M4 | 28 | 1677 | C | T | [symbol] | CDZ96150 |
| X₃₃₂₉ | M4 | 43 | 464 | G | A | [symbol] | CDZ96151 |
| X₃₃₃₀ | M4 | 43 | 753 | C | T | [symbol] | CDZ96151 |
| X₃₃₃₁ | M4 | 43 | 757 | G | A | [symbol] | CDZ96151 |
| X₃₃₃₂ | M4 | 43 | 1527 | C | T | [symbol] | CDZ96151 |
| X₃₃₃₃ | M4 | 43 | 1748 | G | A | [symbol] | CDZ96151 |
| X₃₃₃₄ | M4 | 43 | 1928 | C | T | [symbol] | CDZ96151 |
| X₃₃₃₅ | M4 | 62 | 26 | T | A | [symbol] | CDZ96152 |
| X₃₃₃₆ | M4 | 62 | 27 | T | C | [symbol] | CDZ96152 |
| X₃₃₃₇ | M4 | 62 | 55 | T | A | [symbol] | CDZ96152 |
| X₃₃₃₈ | M4 | 62 | 403 | C | T | [symbol] | CDZ96152 |
| X₃₃₃₉ | M4 | 62 | 875 | G | A | [symbol] | CDZ96152 |
| X₃₃₄₀ | M4 | 102 | 339 | C | T | [symbol] | CDZ96153 |
| X₃₃₄₁ | M4 | 102 | 951 | G | A | [symbol] | CDZ96153 |
| X₃₃₄₂ | M4 | 102 | 1042 | G | A | [symbol] | CDZ96153 |
| X₃₃₄₃ | M4 | 102 | 2334 | G | A | [symbol] | CDZ96153 |
| X₃₃₄₄ | M4 | 102 | 2590 | C | T | [symbol] | CDZ96153 |
| X₃₃₄₅ | M4 | 102 | 3427 | C | T | [symbol] | CDZ96153 |
| X₃₃₄₆ | M4 | 176 | 358 | G | A | [symbol] | CED80058 |
| X₃₃₄₇ | M4 | 176 | 1872 | G | A | [symbol] | CED80058 |
| X₃₃₄₈ | M4 | 176 | 1980 | G | A | [symbol] | CED80058 |
| X₃₃₄₉ | M4 | 176 | 2335 | A | G | [symbol] | CED80058 |
| X₃₃₅₀ | M4 | 176 | 2349 | G | A | [symbol] | CED80058 |
| X₃₃₅₁ | M4 | 179 | 1525 | G | A | [symbol] | CDZ96154 |
| X₃₃₅₂ | M4 | 179 | 1780 | C | T | [symbol] | CDZ96154 |
| X₃₃₅₃ | M4 | 188 | 815 | C | T | [symbol] | CED80060 |
| X₃₃₅₄ | M4 | 188 | 1174 | C | T | [symbol] | CED80060 |
| X₃₃₅₅ | M4 | 188 | 2163 | G | A | [symbol] | CED80060 |
| X₃₃₅₆ | M4 | 188 | 2170 | G | A | [symbol] | CED80060 |
| X₃₃₅₇ | M4 | 247 | 102 | G | A | [symbol] | CED80061 |
| X₃₃₅₈ | M4 | 247 | 989 | C | T | [symbol] | CED80061 |
| X₃₃₅₉ | M4 | 247 | 1053 | G | A | [symbol] | CED80061 |
| X₃₃₆₀ | M4 | 3 | 142 | C | T | [symbol] | |
| X₃₃₆₁ | M4 | 7 | 160 | T | C | [symbol] | |
| X₃₃₆₂ | M4 | 8 | 1110 | C | T | [symbol] | |
| X₃₃₆₃ | M4 | 11 | 154 | A | C | [symbol] | |
| X₃₃₆₄ | M4 | 11 | 158 | C | T | [symbol] | |
| X₃₃₆₅ | M4 | 15 | 28 | C | G | [symbol] | |
| X₃₃₆₆ | M4 | 24 | 513 | T | C | [symbol] | CDZ96160 |
| X₃₃₆₇ | M4 | 24 | 520 | C | G | [symbol] | CDZ96160 |
| X₃₃₆₈ | M4 | 24 | 5850 | C | T | [symbol] | CDZ96160 |
| X₃₃₆₉ | M4 | 24 | 180340 | G | A | [symbol] | CDZ96216 |
| X₃₃₇₀ | M4 | 24 | 216696 | C | T | [symbol] | CDZ96228 |
| X₃₃₇₁ | M4 | 24 | 266697 | G | A | [symbol] | CDZ96242 |
| X₃₃₇₂ | M4 | 24 | 307114 | G | A | [symbol] | CDZ96255 |
| X₃₃₇₃ | M4 | 24 | 340663 | T | C | [symbol] | CDZ96265 |
| X₃₃₇₄ | M4 | 24 | 420108 | G | A | [symbol] | CDZ96291 |
| X₃₃₇₅ | M4 | 24 | 422035 | G | A | [symbol] | CDZ96291 |
| X₃₃₇₆ | M4 | 24 | 470687 | G | A | [symbol] | CDZ96308 |
| X₃₃₇₇ | M4 | 27 | 445 | C | T | [symbol] | |
| X₃₃₇₈ | M4 | 27 | 1012 | A | C | [symbol] | |
| X₃₃₇₉ | M4 | 27 | 1013 | T | A | [symbol] | |
| X₃₃₈₀ | M4 | 32 | 158 | C | T | [symbol] | CDZ96332 |
| X₃₃₈₁ | M4 | 32 | 215 | C | T | [symbol] | CDZ96332 |
| X₃₃₈₂ | M4 | 32 | 2438 | C | T | [symbol] | CDZ96332 |
| X₃₃₈₃ | M4 | 32 | 2734 | C | T | [symbol] | CDZ96332 |
| X₃₃₈₄ | M4 | 32 | 2821 | C | T | [symbol] | CDZ96332 |
| X₃₃₈₅ | M4 | 32 | 3067 | C | T | [symbol] | CDZ96332 |
| X₃₃₈₆ | M4 | 32 | 3506 | C | T | [symbol] | CDZ96332 |
| X₃₃₈₇ | M4 | 32 | 3562 | G | A | [symbol] | CDZ96332 |
| X₃₃₈₈ | M4 | 33 | 399 | T | A | [symbol] | CED82002 |
| X₃₃₈₉ | M4 | 33 | 20227 | G | A | [symbol] | CED82006 |
| X₃₃₉₀ | M4 | 33 | 45488 | C | T | [symbol] | CED82012 |
| X₃₃₉₁ | M4 | 33 | 79913 | G | A | [symbol] | CED82024 |
| X₃₃₉₂ | M4 | 33 | 109397 | G | A | [symbol] | CED82038 |
| X₃₃₉₃ | M4 | 33 | 109494 | G | A | [symbol] | CED82038 |
| X₃₃₉₄ | M4 | 33 | 129733 | G | A | [symbol] | CED82045 |
| X₃₃₉₅ | M4 | 33 | 226363 | G | A | [symbol] | CED82075 |
| X₃₃₉₆ | M4 | 33 | 257786 | C | T | [symbol] | CED82084 |
| X₃₃₉₇ | M4 | 33 | 260027 | G | A | [symbol] | CED82084 |
| X₃₃₉₈ | M4 | 33 | 274627 | G | A | [symbol] | CED82091 |
| X₃₃₉₉ | M4 | 33 | 283808 | C | T | [symbol] | CED82093 |
| X₃₄₀₀ | M4 | 33 | 327840 | C | T | [symbol] | CED82108 |
| X₃₄₀₁ | M4 | 33 | 341890 | G | A | [symbol] | CED82113 |
| X₃₄₀₂ | M4 | 33 | 370630 | A | T | [symbol] | CED82121- |
| | | | | | | | CED82122 |
| X₃₄₀₃ | M4 | 33 | 372141 | A | C | [symbol] | CED82121- |
| | | | | | | | CED82122 |
| X₃₄₀₄ | M4 | 33 | 374131 | A | G | [symbol] | CED82121- |
| | | | | | | | CED82122 |
| X₃₄₀₅ | M4 | 33 | 374134 | A | G | [symbol] | CED82121- |
| | | | | | | | CED82122 |
| X₃₄₀₆ | M4 | 33 | 374137 | A | T | [symbol] | CED82121- |
| | | | | | | | CED82122 |
| X₃₄₀₇ | M4 | 33 | 374150 | A | C | [symbol] | CED82121- |
| | | | | | | | CED82122 |
| X₃₄₀₈ | M4 | 33 | 375339 | A | G | [symbol] | CED82121- |
| | | | | | | | CED82122 |
| X₃₄₀₉ | M4 | 33 | 375366 | T | C | [symbol] | CED82121- |
| | | | | | | | CED82122 |
| X₃₄₁₀ | M4 | 33 | 382577 | C | T | [symbol] | CED82122 |
| X₃₄₁₁ | M4 | 33 | 382832 | G | T | [symbol] | CED82122 |
| X₃₄₁₂ | M4 | 33 | 383212 | G | A | [symbol] | CED82122 |
| X₃₄₁₃ | M4 | 33 | 385319 | A | C | [symbol] | CED82122 |
| X₃₄₁₄ | M4 | 33 | 385527 | C | T | [symbol] | CED82122 |
| X₃₄₁₅ | M4 | 33 | 386225 | G | A | [symbol] | CED82124 |
| X₃₄₁₆ | M4 | 33 | 448815 | G | A | [symbol] | CED82144 |
| X₃₄₁₇ | M4 | 33 | 455451 | C | T | [symbol] | CED82146 |
| X₃₄₁₈ | M4 | 33 | 509127 | C | T | [symbol] | CED82166 |
| X₃₄₁₉ | M4 | 33 | 543070 | C | T | [symbol] | CED82179 |
| X₃₄₂₀ | M4 | 33 | 556726 | C | T | [symbol] | CED82183 |
| X₃₄₂₁ | M4 | 33 | 570390 | G | A | [symbol] | CED82188 |
| X₃₄₂₂ | M4 | 33 | 639242 | G | A | [symbol] | CED82208 |
| X₃₄₂₃ | M4 | 33 | 682740 | C | T | [symbol] | CED82220 |
| X₃₄₂₄ | M4 | 33 | 699423 | C | T | [symbol] | CED82227 |
| X₃₄₂₅ | M4 | 33 | 800536 | G | A | [symbol] | CED82259 |
| X₃₄₂₆ | M4 | 33 | 829825 | C | T | [symbol] | CED82271 |
| X₃₄₂₇ | M4 | 33 | 850373 | G | A | [symbol] | CED82278 |
| X₃₄₂₈ | M4 | 33 | 917771 | A | T | [symbol] | CED82294 |
| X₃₄₂₉ | M4 | 33 | 929585 | T | C | [symbol] | CED82297 |
| X₃₄₃₀ | M4 | 33 | 936571 | A | G | [symbol] | CED82300 |
| X₃₄₃₁ | M4 | 33 | 936600 | G | A | [symbol] | CED82300 |
| X₃₄₃₂ | M4 | 33 | 936667 | A | G | [symbol] | CED82300 |
| X₃₄₃₃ | M4 | 33 | 936697 | A | G | [symbol] | CED82300 |
| X₃₄₃₄ | M4 | 33 | 971503 | C | T | [symbol] | CED82307 |
| X₃₄₃₅ | M4 | 33 | 995886 | G | A | [symbol] | CED82318 |
| X₃₄₃₆ | M4 | 33 | 1005225 | C | T | [symbol] | CED82323 |
| X₃₄₃₇ | M4 | 33 | 1049699 | G | A | [symbol] | CED82334 |
| X₃₄₃₈ | M4 | 33 | 1053098 | G | C | [symbol] | CED82337 |
| X₃₄₃₉ | M4 | 33 | 1087773 | G | A | [symbol] | CED82349 |
| X₃₄₄₀ | M4 | 33 | 1138444 | C | T | [symbol] | CED82363 |
| X₃₄₄₁ | M4 | 33 | 1151553 | C | T | [symbol] | CED82367 |
| X₃₄₄₂ | M4 | 33 | 1165914 | G | A | [symbol] | CED82372 |
| X₃₄₄₃ | M4 | 33 | 1191727 | C | T | [symbol] | CED82379 |
| X₃₄₄₄ | M4 | 33 | 1291620 | C | T | [symbol] | CED82406 |
| X₃₄₄₅ | M4 | 33 | 1311181 | G | A | [symbol] | CED82413 |
| X₃₄₄₆ | M4 | 33 | 1316789 | C | T | [symbol] | CED82415 |
| X₃₄₄₇ | M4 | 33 | 1324395 | G | A | [symbol] | CED82417 |
| X₃₄₄₈ | M4 | 33 | 1339376 | C | T | [symbol] | CED82424 |
| X₃₄₄₉ | M4 | 33 | 1349009 | C | T | [symbol] | CED82427 |
| X₃₄₅₀ | M4 | 33 | 1411059 | C | T | [symbol] | CED82446 |
| X₃₄₅₁ | M4 | 33 | 1431038 | G | A | [symbol] | CED82451 |
| X₃₄₅₂ | M4 | 33 | 1470375 | C | T | [symbol] | CED82465 |
| X₃₄₅₃ | M4 | 33 | 1473759 | G | T | [symbol] | CED82464 |
| X₃₄₅₄ | M4 | 33 | 1499347 | G | A | [symbol] | CED82472 |
| X₃₄₅₅ | M4 | 33 | 1544767 | A | C | [symbol] | CED82485- |
| | | | | | | | CED82486 |
| X₃₄₅₆ | M4 | 33 | 1585699 | C | T | [symbol] | CED82497 |
| X₃₄₅₇ | M4 | 33 | 1587443 | C | T | [symbol] | CED82497 |
| X₃₄₅₈ | M4 | 33 | 1596242 | C | T | [symbol] | CED82498 |
| X₃₄₅₉ | M4 | 33 | 1638726 | G | A | [symbol] | CED82513 |
| X₃₄₆₀ | M4 | 33 | 1642659 | G | A | [symbol] | CED82515 |
| X₃₄₆₁ | M4 | 33 | 1652749 | C | T | [symbol] | CED82516 |
| X₃₄₆₂ | M4 | 33 | 1669434 | G | A | [symbol] | CED82524 |
| X₃₄₆₃ | M4 | 33 | 1700000 | G | A | [symbol] | CED82534 |
| X₃₄₆₄ | M4 | 33 | 1728730 | G | A | [symbol] | CED82541 |
| X₃₄₆₅ | M4 | 33 | 1740595 | C | T | [symbol] | CED82545 |
| X₃₄₆₆ | M4 | 33 | 1752414 | G | A | [symbol] | CED82547 |
| X₃₄₆₇ | M4 | 33 | 1823362 | C | T | [symbol] | CED82571 |
| X₃₄₆₈ | M4 | 33 | 1824401 | C | T | [symbol] | CED82572 |
| X₃₄₆₉ | M4 | 33 | 1860278 | G | A | [symbol] | CED82582 |
| X₃₄₇₀ | M4 | 33 | 1861675 | C | T | [symbol] | CED82583 |
| X₃₄₇₁ | M4 | 33 | 1862200 | G | A | [symbol] | CED82582 |
| X₃₄₇₂ | M4 | 33 | 1887401 | C | T | [symbol] | CED82591 |
| X₃₄₇₃ | M4 | 33 | 1889585 | G | A | [symbol] | CED82592 |
| X₃₄₇₄ | M4 | 33 | 1908405 | G | A | [symbol] | CED82599 |
| X₃₄₇₅ | M4 | 33 | 1938614 | G | A | [symbol] | CED82612 |
| X₃₄₇₆ | M4 | 33 | 1939670 | C | T | [symbol] | CED82612 |
| X₃₄₇₇ | M4 | 33 | 1967112 | C | T | [symbol] | CED82622 |
| X₃₄₇₈ | M4 | 33 | 2010230 | C | T | [symbol] | CED82636 |
| X₃₄₇₉ | M4 | 33 | 2050847 | G | A | [symbol] | CED82651 |
| X₃₄₈₀ | M4 | 36 | 320 | G | A | [symbol] | |
| X₃₄₈₁ | M4 | 36 | 1056 | G | A | [symbol] | |
| X₃₄₈₂ | M4 | 36 | 1295 | C | T | [symbol] | |
| X₃₄₈₃ | M4 | 36 | 1461 | A | T | [symbol] | |
| X₃₄₈₄ | M4 | 40 | 495 | C | G | [symbol] | |
| X₃₄₈₅ | M4 | 42 | 804 | C | T | [symbol] | CDZ96333 |
| X₃₄₈₆ | M4 | 42 | 1559 | G | A | [symbol] | CDZ96333 |
| X₃₄₈₇ | M4 | 48 | 563 | C | T | [symbol] | |
| X₃₄₈₈ | M4 | 48 | 565 | T | C | [symbol] | |
| X₃₄₈₉ | M4 | 48 | 576 | G | A | [symbol] | |
| X₃₄₉₀ | M4 | 48 | 580 | T | A | [symbol] | |
| X₃₄₉₁ | M4 | 50 | 145 | G | A | [symbol] | |
| X₃₄₉₂ | M4 | 52 | 6285 | C | T | [symbol] | CED82665 |
| X₃₄₉₃ | M4 | 52 | 136058 | G | A | [symbol] | CED82705 |
| X₃₄₉₄ | M4 | 52 | 144382 | C | T | [symbol] | CED82709 |
| X₃₄₉₅ | M4 | 52 | 145060 | G | A | [symbol] | CED82708 |
| X₃₄₉₆ | M4 | 52 | 151974 | C | T | [symbol] | CED82712 |
| X₃₄₉₇ | M4 | 52 | 183986 | C | T | [symbol] | CED82722 |
| X₃₄₉₈ | M4 | 52 | 203253 | A | G | [symbol] | CED82730 |
| X₃₄₉₉ | M4 | 52 | 207354 | C | T | [symbol] | CED82730 |
| X₃₅₀₀ | M4 | 52 | 223283 | C | G | [symbol] | CED82732- |
| | | | | | | | CED82733 |
| X₃₅₀₁ | M4 | 52 | 239773 | C | T | [symbol] | CED82734 |
| X₃₅₀₂ | M4 | 52 | 243203 | G | A | [symbol] | CED82733 |
| X₃₅₀₃ | M4 | 52 | 244305 | G | A | [symbol] | CED82734 |
| X₃₅₀₄ | M4 | 52 | 284733 | G | A | [symbol] | CED82747 |
| X₃₅₀₅ | M4 | 52 | 390095 | G | A | [symbol] | CED82784 |
| X₃₅₀₆ | M4 | 52 | 390988 | C | T | [symbol] | CED82785 |
| X₃₅₀₇ | M4 | 52 | 395374 | G | A | [symbol] | CED82786 |
| X₃₅₀₈ | M4 | 52 | 398723 | C | T | [symbol] | CED82786 |
| X₃₅₀₉ | M4 | 52 | 398728 | C | T | [symbol] | CED82786 |
| X₃₅₁₀ | M4 | 52 | 487472 | C | T | [symbol] | CED82814 |
| X₃₅₁₁ | M4 | 52 | 516267 | C | T | [symbol] | CED82823 |
| X₃₅₁₂ | M4 | 52 | 552099 | G | A | [symbol] | CED82837 |
| X₃₅₁₃ | M4 | 52 | 552173 | G | A | [symbol] | CED82837 |
| X₃₅₁₄ | M4 | 52 | 571124 | G | A | [symbol] | CED82845 |
| X₃₅₁₅ | M4 | 52 | 627827 | C | T | [symbol] | CED82860 |
| X₃₅₁₆ | M4 | 52 | 638622 | G | A | [symbol] | CED82862 |
| X₃₅₁₇ | M4 | 52 | 679670 | G | A | [symbol] | CED82877 |
| X₃₅₁₈ | M4 | 52 | 730557 | G | T | [symbol] | CED82899 |
| X₃₅₁₉ | M4 | 52 | 750413 | G | A | [symbol] | CED82908 |
| X₃₅₂₀ | M4 | 52 | 817452 | T | A | [symbol] | CED82928 |
| X₃₅₂₁ | M4 | 52 | 840755 | C | T | [symbol] | CED82935 |
| X₃₅₂₂ | M4 | 52 | 842352 | C | T | [symbol] | CED82934 |
| X₃₅₂₃ | M4 | 52 | 848043 | A | G | [symbol] | CED82937 |
| X₃₅₂₄ | M4 | 52 | 849671 | G | A | [symbol] | CED82940 |
| X₃₅₂₅ | M4 | 52 | 861376 | C | T | [symbol] | CED82946 |
| X₃₅₂₆ | M4 | 52 | 911438 | G | A | [symbol] | CED82964 |
| X₃₅₂₇ | M4 | 52 | 928854 | C | T | [symbol] | CED82971 |
| X₃₅₂₈ | M4 | 52 | 938650 | G | A | [symbol] | CED82973 |
| X₃₅₂₉ | M4 | 52 | 967316 | A | T | [symbol] | CED82984 |
| X₃₅₃₀ | M4 | 52 | 967701 | T | A | [symbol] | CED82983 |
| X₃₅₃₁ | M4 | 52 | 977048 | G | A | [symbol] | CED82990 |
| X₃₅₃₂ | M4 | 52 | 1003899 | G | A | [symbol] | CED83001 |
| X₃₅₃₃ | M4 | 52 | 1004724 | G | A | [symbol] | CED83001 |
| X₃₅₃₄ | M4 | 52 | 1029498 | G | A | [symbol] | CED83007 |
| X₃₅₃₅ | M4 | 52 | 1043011 | C | T | [symbol] | CED83012 |
| X₃₅₃₆ | M4 | 52 | 1049304 | C | T | [symbol] | CED83013 |
| X₃₅₃₇ | M4 | 52 | 1067895 | C | T | [symbol] | CED83020 |
| X₃₅₃₈ | M4 | 52 | 1157819 | T | A | [symbol] | CED83049 |
| X₃₅₃₉ | M4 | 52 | 1158103 | C | T | [symbol] | CED83051 |
| X₃₅₄₀ | M4 | 52 | 1214927 | C | T | [symbol] | CED83068 |
| X₃₅₄₁ | M4 | 52 | 1219894 | C | T | [symbol] | CED83071 |
| X₃₅₄₂ | M4 | 52 | 1227910 | G | A | [symbol] | CED83074 |
| X₃₅₄₃ | M4 | 52 | 1297876 | G | A | [symbol] | CED83099 |
| X₃₅₄₄ | M4 | 52 | 1336560 | G | A | [symbol] | CED83113 |
| X₃₅₄₅ | M4 | 52 | 1356590 | C | T | [symbol] | CED83120 |
| X₃₅₄₆ | M4 | 52 | 1363357 | G | A | [symbol] | CED83122 |
| X₃₅₄₇ | M4 | 52 | 1363405 | C | T | [symbol] | CED83123 |
| X₃₅₄₈ | M4 | 52 | 1373998 | C | T | [symbol] | CED83125 |
| X₃₅₄₉ | M4 | 52 | 1379155 | G | A | [symbol] | CED83128 |
| X₃₅₅₀ | M4 | 52 | 1381652 | C | T | [symbol] | CED83129 |
| X₃₅₅₁ | M4 | 52 | 1399439 | G | A | [symbol] | CED83134 |
| X₃₅₅₂ | M4 | 52 | 1409725 | G | A | [symbol] | CED83139 |
| X₃₅₅₃ | M4 | 52 | 1474412 | G | A | [symbol] | CED83166 |
| X₃₅₅₄ | M4 | 52 | 1509004 | G | A | [symbol] | CED83177 |
| X₃₅₅₅ | M4 | 52 | 1526796 | G | A | [symbol] | CED83181 |
| X₃₅₅₆ | M4 | 52 | 1540879 | C | T | [symbol] | CED83186 |
| X₃₅₅₇ | M4 | 52 | 1546812 | G | A | [symbol] | CED83188 |
| X₃₅₅₈ | M4 | 52 | 1570053 | C | T | [symbol] | CED83200 |
| X₃₅₅₉ | M4 | 52 | 1583655 | C | T | [symbol] | CED83206 |
| X₃₅₆₀ | M4 | 52 | 1590982 | C | T | [symbol] | CED83207 |
| X₃₅₆₁ | M4 | 52 | 1708900 | A | G | [symbol] | CED83249 |
| X₃₅₆₂ | M4 | 52 | 1715136 | C | T | [symbol] | CED83250 |
| X₃₅₆₃ | M4 | 52 | 1716210 | C | T | [symbol] | CED83250 |
| X₃₅₆₄ | M4 | 52 | 1750963 | C | T | [symbol] | CED83263 |
| X₃₅₆₅ | M4 | 52 | 1760440 | G | A | [symbol] | CED83266 |
| X₃₅₆₆ | M4 | 52 | 1765533 | G | A | [symbol] | CED83268 |
| X₃₅₆₇ | M4 | 52 | 1787135 | C | T | [symbol] | CED83275 |
| X₃₅₆₈ | M4 | 52 | 1788070 | G | A | [symbol] | CED83273 |
| X₃₅₆₉ | M4 | 52 | 1789922 | G | A | [symbol] | CED83276 |
| X₃₅₇₀ | M4 | 52 | 1793370 | G | A | [symbol] | CED83275 |
| X₃₅₇₁ | M4 | 52 | 1807309 | G | A | [symbol] | CED83283 |
| X₃₅₇₂ | M4 | 52 | 1809897 | G | A | [symbol] | CED83285 |
| X₃₅₇₃ | M4 | 52 | 1871310 | C | T | [symbol] | CED83304 |
| X₃₅₇₄ | M4 | 52 | 1874150 | G | T | [symbol] | CED83305 |
| X₃₅₇₅ | M4 | 52 | 1893352 | G | A | [symbol] | CED83313 |
| X₃₅₇₆ | M4 | 52 | 1938393 | C | T | [symbol] | CED83322 |
| X₃₅₇₇ | M4 | 52 | 1963875 | G | A | [symbol] | CED83335 |
| X₃₅₇₈ | M4 | 52 | 1987096 | G | A | [symbol] | CED83341 |
| X₃₅₇₉ | M4 | 52 | 1995167 | G | A | [symbol] | CED83342 |
| X₃₅₈₀ | M4 | 52 | 2041781 | C | T | [symbol] | CED83355 |
| X₃₅₈₁ | M4 | 52 | 2050245 | G | A | [symbol] | CED83359 |
| X₃₅₈₂ | M4 | 52 | 2081390 | G | A | [symbol] | CED83367 |
| X₃₅₈₃ | M4 | 52 | 2093251 | C | T | [symbol] | CED83368 |
| X₃₅₈₄ | M4 | 52 | 2120665 | C | T | [symbol] | CED83376 |
| X₃₅₈₅ | M4 | 52 | 2136885 | A | G | [symbol] | CED83382 |
| X₃₅₈₆ | M4 | 52 | 2162878 | G | A | [symbol] | CED83392 |
| X₃₅₈₇ | M4 | 52 | 2192445 | G | A | [symbol] | CED83400 |
| X₃₅₈₈ | M4 | 52 | 2211680 | G | A | [symbol] | CED83407 |
| X₃₅₈₉ | M4 | 52 | 2222126 | C | T | [symbol] | CED83410 |
| X₃₅₉₀ | M4 | 52 | 2229000 | G | A | [symbol] | CED83410 |
| X₃₅₉₁ | M4 | 52 | 2265219 | C | T | [symbol] | CED83425 |
| X₃₅₉₂ | M4 | 52 | 2266378 | C | T | [symbol] | CED83423 |
| X₃₅₉₃ | M4 | 52 | 2279651 | G | A | [symbol] | CED83430 |
| X₃₅₉₁ | M4 | 52 | 2308421 | T | C | [symbol] | CED83439 |
| X₃₅₉₅ | M4 | 52 | 2327266 | C | T | [symbol] | CED83445 |
| X₃₅₉₆ | M4 | 52 | 2364315 | G | A | [symbol] | CED83455 |
| X₃₅₉₇ | M4 | 52 | 2374740 | C | T | [symbol] | CED83456 |
| X₃₅₉₈ | M4 | 52 | 2400778 | C | T | [symbol] | CED83463 |
| X₃₅₉₉ | M4 | 52 | 2443575 | G | A | [symbol] | CED83476 |
| X₃₆₀₀ | M4 | 52 | 2531584 | C | T | [symbol] | CED83507 |
| X₃₆₀₁ | M4 | 52 | 2575090 | A | G | [symbol] | CED83525 |
| X₃₆₀₂ | M4 | 53 | 26291 | G | A | [symbol] | CDZ96348 |
| X₃₆₀₃ | M4 | 53 | 55702 | C | T | [symbol] | CDZ96359 |
| X₃₆₀₄ | M4 | 53 | 69664 | C | T | [symbol] | CDZ96363 |
| X₃₆₀₅ | M4 | 54 | 4150 | A | G | [symbol] | CDZ96383 |
| X₃₆₀₆ | M4 | 54 | 4815 | G | A | [symbol] | CDZ96384 |
| X₃₆₀₇ | M4 | 54 | 4816 | A | T | [symbol] | CDZ96384 |
| X₃₆₀₈ | M4 | 54 | 4817 | G | C | [symbol] | CDZ96384 |
| X₃₆₀₉ | M4 | 54 | 4821 | G | A | [symbol] | CDZ96384 |
| X₃₆₁₀ | M4 | 54 | 16963 | C | T | [symbol] | CDZ96386 |
| X₃₆₁₁ | M4 | 54 | 60769 | G | A | [symbol] | CDZ96403 |
| X₃₆₁₂ | M4 | 54 | 87716 | G | A | [symbol] | CDZ96410 |
| X₃₆₁₃ | M4 | 54 | 106086 | G | A | [symbol] | CDZ96416 |
| X₃₆₁₄ | M4 | 54 | 114442 | G | A | [symbol] | CDZ96419 |
| X₃₆₁₅ | M4 | 54 | 136832 | C | T | [symbol] | CDZ96425 |
| X₃₆₁₆ | M4 | 54 | 194230 | G | A | [symbol] | CDZ96446 |
| X₃₆₁₇ | M4 | 54 | 195804 | G | A | [symbol] | CDZ96447 |
| X₃₆₁₈ | M4 | 54 | 202467 | C | T | [symbol] | CDZ96448 |
| X₃₆₁₉ | M4 | 54 | 206494 | G | A | [symbol] | CDZ96450 |
| X₃₆₂₀ | M4 | 54 | 274617 | A | T | [symbol] | CDZ96471 |
| X₃₆₂₁ | M4 | 54 | 278479 | C | T | [symbol] | CDZ96474 |
| X₃₆₂₂ | M4 | 54 | 285008 | C | T | [symbol] | CDZ96475 |
| X₃₆₂₃ | M4 | 54 | 370320 | G | A | [symbol] | CDZ96499 |
| X₃₆₂₄ | M4 | 54 | 383172 | C | T | [symbol] | CDZ96506 |
| X₃₆₂₅ | M4 | 54 | 391223 | A | G | [symbol] | CDZ96509 |
| X₃₆₂₆ | M4 | 54 | 427542 | C | T | [symbol] | CDZ96520 |
| X₃₆₂₇ | M4 | 54 | 478527 | C | T | [symbol] | CDZ96537 |
| X₃₆₂₈ | M4 | 54 | 537282 | T | C | [symbol] | CDZ96557 |
| X₃₆₂₉ | M4 | 54 | 564688 | C | T | [symbol] | CDZ96566 |
| X₃₆₃₀ | M4 | 54 | 565396 | T | C | [symbol] | CDZ96567 |
| X₃₆₃₁ | M4 | 54 | 583996 | G | A | [symbol] | CDZ96570 |
| X₃₆₃₂ | M4 | 54 | 616268 | A | G | [symbol] | CDZ96581 |
| X₃₆₃₃ | M4 | 54 | 619019 | G | A | [symbol] | CDZ96580 |
| X₃₆₃₄ | M4 | 54 | 733193 | T | C | [symbol] | CDZ96619 |
| X₃₆₃₅ | M4 | 54 | 742842 | C | T | [symbol] | CDZ96621 |
| X₃₆₃₆ | M4 | 54 | 743396 | G | A | [symbol] | CDZ96621 |
| X₃₆₃₇ | M4 | 54 | 746707 | G | A | [symbol] | CDZ96623 |
| X₃₆₃₈ | M4 | 58 | 93 | T | C | [symbol] | |
| X₃₆₃₉ | M4 | 64 | 2756 | A | C | [symbol] | CDZ96626 |
| X₃₆₄₀ | M4 | 67 | 111 | G | A | [symbol] | |
| X₃₆₄₁ | M4 | 69 | 29006 | G | A | [symbol] | CED83534 |
| X₃₆₄₂ | M4 | 69 | 48869 | G | A | [symbol] | CED83542 |
| X₃₆₄₃ | M4 | 69 | 50642 | C | T | [symbol] | CED83543 |
| X₃₆₄₄ | M4 | 69 | 52428 | G | A | [symbol] | CED83541 |
| X₃₆₄₅ | M4 | 69 | 70304 | C | T | [symbol] | CED83551 |
| X₃₆₄₆ | M4 | 69 | 89393 | G | A | [symbol] | CED83556 |
| X₃₆₄₇ | M4 | 69 | 107920 | C | T | [symbol] | CED83561 |
| X₃₆₄₈ | M4 | 69 | 201005 | G | A | [symbol] | CED83595 |
| X₃₆₄₉ | M4 | 69 | 293602 | G | A | [symbol] | CED83626 |
| X₃₆₅₀ | M4 | 69 | 384418 | G | A | [symbol] | CED83656 |
| X₃₆₅₁ | M4 | 69 | 415659 | C | T | [symbol] | CED83665 |
| X₃₆₅₂ | M4 | 69 | 431248 | G | A | [symbol] | CED83671 |
| X₃₆₅₃ | M4 | 69 | 441238 | C | G | [symbol] | CED83674 |
| X₃₆₅₄ | M4 | 69 | 455639 | G | A | [symbol] | CED83677 |
| X₃₆₅₅ | M4 | 69 | 461986 | G | A | [symbol] | CED83680 |
| X₃₆₅₆ | M4 | 69 | 472563 | C | T | [symbol] | CED83681 |
| X₃₆₅₇ | M4 | 69 | 473113 | G | A | [symbol] | CED83684 |
| X₃₆₅₈ | M4 | 69 | 482073 | G | T | [symbol] | CED83685 |
| X₃₆₅₉ | M4 | 69 | 585845 | G | A | [symbol] | CED83722 |
| X₃₆₆₀ | M4 | 69 | 631511 | G | A | [symbol] | CED83742 |
| X₃₆₆₁ | M4 | 69 | 637299 | T | C | [symbol] | CED83744 |
| X₃₆₆₂ | M4 | 69 | 675579 | G | A | [symbol] | CED83760 |
| X₃₆₆₃ | M4 | 69 | 676581 | G | A | [symbol] | CED83760 |
| X₃₆₆₄ | M4 | 69 | 676853 | G | A | [symbol] | CED83759 |
| X₃₆₆₅ | M4 | 69 | 678406 | C | T | [symbol] | CED83759 |
| X₃₆₆₆ | M4 | 69 | 682770 | C | T | [symbol] | CED83763 |
| X₃₆₆₇ | M4 | 69 | 803358 | G | A | [symbol] | CED83798 |
| X₃₆₆₈ | M4 | 69 | 865820 | C | T | [symbol] | CED83814 |
| X₃₆₆₉ | M4 | 69 | 869706 | G | A | [symbol] | CED83815 |
| X₃₆₇₀ | M4 | 69 | 892199 | G | A | [symbol] | CED83822 |
| X₃₆₇₁ | M4 | 69 | 894462 | C | T | [symbol] | CED83821 |
| X₃₆₇₂ | M4 | 69 | 949982 | C | T | [symbol] | CED83842 |
| X₃₆₇₃ | M4 | 69 | 971219 | C | T | [symbol] | CED83847 |
| X₃₆₇₄ | M4 | 69 | 988364 | G | A | [symbol] | CED83857 |
| X₃₆₇₅ | M4 | 69 | 1038140 | G | A | [symbol] | CED83874 |
| X₃₆₇₆ | M4 | 69 | 1042212 | G | A | [symbol] | CED83875 |
| X₃₆₇₇ | M4 | 69 | 1080212 | C | T | [symbol] | CED83889 |
| X₃₆₇₈ | M4 | 69 | 1084824 | G | A | [symbol] | CED83891 |
| X₃₆₇₉ | M4 | 69 | 1084835 | C | T | [symbol] | CED83891 |
| X₃₆₈₀ | M4 | 69 | 1084848 | A | G | [symbol] | CED83891 |
| X₃₆₈₁ | M4 | 69 | 1084866 | A | G | [symbol] | CED83891 |
| X₃₆₈₂ | M4 | 69 | 1084931 | C | T | [symbol] | CED83891 |
| X₃₆₈₃ | M4 | 69 | 1102164 | C | T | [symbol] | CED83897 |
| X₃₆₈₄ | M4 | 69 | 1109785 | C | T | [symbol] | CED83899 |
| X₃₆₈₅ | M4 | 69 | 1109813 | A | G | [symbol] | CED83899 |
| X₃₆₈₆ | M4 | 69 | 1109818 | A | G | [symbol] | CED83899 |
| X₃₆₈₇ | M4 | 69 | 1109819 | T | A | [symbol] | CED83899 |
| X₃₆₈₈ | M4 | 69 | 1109823 | T | C | [symbol] | CED83899 |
| X₃₆₈₉ | M4 | 69 | 1109830 | A | G | [symbol] | CED83899 |
| X₃₆₉₀ | M4 | 69 | 1109831 | C | A | [symbol] | CED83899 |
| X₃₆₉₁ | M4 | 69 | 1109835 | A | G | [symbol] | CED83899 |
| X₃₆₉₂ | M4 | 69 | 1110489 | T | A | [symbol] | CED83899 |
| X₃₆₉₃ | M4 | 69 | 1110507 | A | T | [symbol] | CED83899 |
| X₃₆₉₄ | M4 | 69 | 1110521 | A | C | [symbol] | CED83899 |
| X₃₆₉₅ | M4 | 69 | 1115486 | C | T | [symbol] | CED83902 |
| X₃₆₉₆ | M4 | 69 | 1152012 | g | A | [symbol] | CED83915 |
| X₃₆₉₇ | M4 | 69 | 1161041 | A | G | [symbol] | CED83918 |
| X₃₆₉₈ | M4 | 69 | 1205454 | C | T | [symbol] | CED83932 |
| X₃₆₉₉ | M4 | 69 | 1218409 | G | A | [symbol] | CED83936 |
| X₃₇₀₀ | M4 | 69 | 1245509 | C | T | [symbol] | CED83943 |
| X₃₇₀₁ | M4 | 69 | 1260087 | G | A | [symbol] | CED83949 |
| X₃₇₀₂ | M4 | 69 | 1342909 | G | A | [symbol] | CED83975 |
| X₃₇₀₃ | M4 | 69 | 1356844 | C | t | [symbol] | CED83982 |
| X₃₇₀₄ | M4 | 69 | 1372486 | C | T | [symbol] | CED83987 |
| X₃₇₀₅ | M4 | 69 | 1379683 | C | T | [symbol] | CED83991 |
| X₃₇₀₆ | M4 | 69 | 1411237 | C | T | [symbol] | CED84001 |
| X₃₇₀₇ | M4 | 69 | 1411421 | C | T | [symbol] | CED83999 |
| X₃₇₀₈ | M4 | 69 | 1421240 | C | T | [symbol] | CED84004 |
| X₃₇₀₉ | M4 | 69 | 1430120 | C | T | [symbol] | CED84006 |
| X₃₇₁₀ | M4 | 69 | 1485040 | G | A | [symbol] | CED84023 |
| X₃₇₁₁ | M4 | 69 | 1493453 | C | T | [symbol] | CED84026 |
| X₃₇₁₂ | M4 | 69 | 1590302 | G | A | [symbol] | CED84059 |
| X₃₇₁₃ | M4 | 69 | 1601173 | c | T | [symbol] | CED84062 |
| X₃₇₁₄ | M4 | 69 | 1636638 | C | T | [symbol] | CED84073 |
| X₃₇₁₅ | M4 | 69 | 1652288 | C | T | [symbol] | CED84077 |
| X₃₇₁₆ | M4 | 69 | 1674261 | C | T | [symbol] | CED84084 |
| X₃₇₁₇ | M4 | 69 | 1688555 | C | T | [symbol] | CED84088 |
| X₃₇₁₈ | M4 | 69 | 1694047 | C | T | [symbol] | CED84089 |
| X₃₇₁₉ | M4 | 69 | 1714728 | G | A | [symbol] | CED84097 |
| X₃₇₂₀ | M4 | 69 | 1790823 | C | T | [symbol] | CED84125 |
| X₃₇₂₁ | m4 | 69 | 1792597 | C | T | [symbol] | CED84126 |
| X₃₇₂₂ | M4 | 69 | 1800584 | G | A | [symbol] | CED84129 |
| X₃₇₂₃ | M4 | 69 | 1853510 | T | C | [symbol] | CED84145 |
| X₃₇₂₄ | M4 | 69 | 1854739 | G | A | [symbol] | CED84145 |
| X₃₇₂₅ | M4 | 69 | 1924621 | C | T | [symbol] | CED84172 |
| X₃₇₂₆ | M4 | 69 | 1967529 | C | T | [symbol] | CED84189 |
| X₃₇₂₇ | M4 | 69 | 1968848 | A | G | [symbol] | CED84189 |
| X₃₇₂₈ | M4 | 69 | 2001469 | C | T | [symbol] | CED84197 |
| X₃₇₂₉ | M4 | 69 | 2009058 | G | A | [symbol] | CED84201 |
| X₃₇₃₀ | M4 | 69 | 2031440 | G | A | [symbol] | CED84210 |
| X₃₇₃₁ | M4 | 69 | 2038266 | G | A | [symbol] | CED84211 |
| X₃₇₃₂ | M4 | 69 | 2099556 | C | T | [symbol] | CED84233 |
| X₃₇₃₃ | M4 | 69 | 2099559 | A | T | [symbol] | CED84233 |
| X₃₇₃₄ | M4 | 69 | 2113590 | G | A | [symbol] | CED84237 |
| X₃₇₃₅ | M4 | 69 | 2156050 | C | T | [symbol] | CED84252 |
| X₃₇₃₆ | M4 | 69 | 2160207 | C | T | [symbol] | CED84255 |
| X₃₇₃₇ | M4 | 69 | 2219444 | G | A | [symbol] | CED84271 |
| X₃₇₃₈ | M4 | 69 | 2226113 | C | T | [symbol] | CED84272 |
| X₃₇₃₉ | M4 | 69 | 2226317 | C | T | [symbol] | CED84272 |
| X₃₇₄₀ | M4 | 69 | 2260222 | C | T | [symbol] | CED84282 |
| X₃₇₄₁ | M4 | 69 | 2275703 | G | A | [symbol] | CED84290 |
| X₃₇₄₂ | M4 | 69 | 2402826 | C | T | [symbol] | CED84333 |
| X₃₇₄₃ | M4 | 69 | 2438727 | C | T | [symbol] | CED84343 |
| X₃₇₄₄ | M4 | 69 | 2440765 | C | A | [symbol] | CED84343 |
| X₃₇₄₅ | M4 | 69 | 2448630 | C | T | [symbol] | CED84344 |
| X₃₇₄₆ | M4 | 69 | 2483433 | G | A | [symbol] | CED84352 |
| X₃₇₄₇ | M4 | 72 | 93 | T | C | [symbol] | |
| X₃₇₄₈ | M4 | 72 | 584 | G | A | [symbol] | |
| X₃₇₄₉ | M4 | 72 | 856 | C | T | [symbol] | |
| X₃₇₅₀ | M4 | 72 | 1163 | C | T | [symbol] | |
| X₃₇₅₁ | M4 | 77 | 18912 | A | G | [symbol] | CDZ96635 |
| X₃₇₅₂ | M4 | 77 | 34978 | G | T | [symbol] | CDZ96642 |
| X₃₇₅₃ | M4 | 77 | 39842 | G | A | [symbol] | CDZ96646 |
| X₃₇₅₄ | M4 | 77 | 65419 | G | A | [symbol] | CDZ96655 |
| X₃₇₅₅ | M4 | 77 | 84022 | G | A | [symbol] | CDZ96659 |
| X₃₇₅₆ | M4 | 78 | 1865 | C | T | [symbol] | CED84359 |
| X₃₇₅₇ | M4 | 78 | 6333 | G | A | [symbol] | CED84360 |
| X₃₇₅₈ | M4 | 78 | 21716 | C | T | [symbol] | CED84364 |
| X₃₇₅₉ | M4 | 78 | 89958 | C | T | [symbol] | CED84387 |
| X₃₇₆₀ | M4 | 78 | 158534 | G | A | [symbol] | CED84406 |
| X₃₇₆₁ | M4 | 78 | 191358 | G | A | [symbol] | CED84414 |
| X₃₇₆₂ | M4 | 78 | 233543 | T | C | [symbol] | CED84431 |
| X₃₇₆₃ | M4 | 78 | 238854 | C | T | [symbol] | CED84430 |
| X₃₇₆₄ | M4 | 78 | 263828 | T | C | [symbol] | CED84438 |
| X₃₇₆₅ | M4 | 78 | 281454 | C | A | [symbol] | CED84441 |
| X₃₇₆₆ | M4 | 78 | 298141 | C | G | [symbol] | CED84443 |
| X₃₇₆₇ | M4 | 78 | 299644 | G | A | [symbol] | CED84443 |
| X₃₇₆₈ | M4 | 78 | 301815 | T | C | [symbol] | CED84443 |
| X₃₇₆₉ | M4 | 78 | 302700 | A | G | [symbol] | CED84443 |
| X₃₇₇₀ | M4 | 78 | 333312 | A | G | [symbol] | CED84452 |
| X₃₇₇₁ | M4 | 78 | 415432 | G | A | [symbol] | CED84479 |
| X₃₇₇₂ | M4 | 78 | 444292 | C | T | [symbol] | CED84490 |
| X₃₇₇₃ | M4 | 78 | 462728 | G | A | [symbol] | CED84497 |
| X₃₇₇₄ | M4 | 78 | 506703 | G | A | [symbol] | CED84508 |
| X₃₇₇₅ | M4 | 78 | 626958 | C | A | [symbol] | CED84549 |
| X₃₇₇₆ | M4 | 78 | 631891 | C | T | [symbol] | CED84551 |
| X₃₇₇₇ | M4 | 78 | 658932 | G | A | [symbol] | CED84561 |
| X₃₇₇₈ | M4 | 78 | 708376 | C | T | [symbol] | CED84580 |
| X₃₇₇₉ | M4 | 78 | 715700 | G | A | [symbol] | CED84582 |
| X₃₇₈₀ | M4 | 78 | 763771 | G | A | [symbol] | CED84600 |
| X₃₇₈₁ | M4 | 78 | 774252 | G | A | [symbol] | CED84602 |
| X₃₇₈₂ | M4 | 78 | 875825 | C | T | [symbol] | CED84633 |
| X₃₇₈₃ | M4 | 78 | 895505 | C | T | [symbol] | CED84642 |
| X₃₇₈₄ | M4 | 78 | 904287 | G | A | [symbol] | CED84646 |
| X₃₇₈₅ | M4 | 78 | 905457 | A | G | [symbol] | CED84645 |
| X₃₇₈₆ | M4 | 78 | 952815 | G | A | [symbol] | CED84662 |
| X₃₇₈₇ | M4 | 78 | 952860 | G | A | [symbol] | CED84662 |
| X₃₇₈₈ | M4 | 78 | 1023381 | G | A | [symbol] | CED84688 |
| X₃₇₈₉ | M4 | 78 | 1067609 | G | A | [symbol] | CED84705 |
| X₃₇₉₀ | M4 | 78 | 1086537 | C | T | [symbol] | CED84713 |
| X₃₇₉₁ | M4 | 78 | 1095322 | C | T | [symbol] | CED84715 |
| X₃₇₉₂ | M4 | 78 | 1111002 | G | A | [symbol] | CED84721 |
| X₃₇₉₃ | M4 | 78 | 1115511 | G | A | [symbol] | CED84723 |
| X₃₇₉₄ | M4 | 78 | 1118856 | C | T | [symbol] | CED84724 |
| X₃₇₉₅ | M4 | 78 | 1199323 | C | T | [symbol] | CED84746 |
| X₃₇₉₆ | M4 | 78 | 1213556 | G | A | [symbol] | CED84752 |
| X₃₇₉₇ | M4 | 78 | 1216573 | C | T | [symbol] | CED84752 |
| X₃₇₉₈ | M4 | 78 | 1247678 | C | T | [symbol] | CED84761 |
| X₃₇₉₉ | M4 | 78 | 1302286 | C | T | [symbol] | CED84782 |
| X₃₈₀₀ | M4 | 78 | 1315648 | C | T | [symbol] | CED84788 |
| X₃₈₀₁ | M4 | 78 | 1317124 | G | A | [symbol] | CED84787 |
| X₃₈₀₂ | M4 | 78 | 1344640 | C | T | [symbol] | CED84795 |
| X₃₈₀₃ | M4 | 78 | 1360288 | G | A | [symbol] | CED84802 |
| X₃₈₀₄ | M4 | 78 | 1382780 | G | A | [symbol] | CED84812 |
| X₃₈₀₅ | M4 | 78 | 1397982 | G | A | [symbol] | CED84818 |
| X₃₈₀₆ | M4 | 78 | 1410781 | G | A | [symbol] | CED84825 |
| X₃₈₀₇ | M4 | 78 | 1426722 | C | T | [symbol] | CED84829 |
| X₃₈₀₈ | M4 | 78 | 1475803 | G | A | [symbol] | CED84845 |
| X₃₈₀₉ | M4 | 78 | 1532973 | G | A | [symbol] | CED84857 |
| X₃₈₁₀ | M4 | 78 | 1563246 | C | T | [symbol] | CED84865 |
| X₃₈₁₁ | M4 | 78 | 1568326 | C | T | [symbol] | CED84868 |
| X₃₈₁₂ | M4 | 78 | 1578844 | G | A | [symbol] | CED84871 |
| X₃₈₁₃ | M4 | 78 | 1609850 | C | T | [symbol] | CED84880 |
| X₃₈₁₄ | M4 | 78 | 1642170 | G | A | [symbol] | CED84889 |
| X₃₈₁₅ | M4 | 78 | 1660408 | G | A | [symbol] | CED84897 |
| X₃₈₁₆ | M4 | 78 | 1673601 | C | T | [symbol] | CED84903 |
| X₃₈₁₇ | M4 | 78 | 1709344 | G | A | [symbol] | CED84917 |
| X₃₈₁₈ | M4 | 78 | 1742796 | G | A | [symbol] | CED84926 |
| X₃₈₁₉ | M4 | 79 | 65 | G | A | [symbol] | CDZ96674 |
| X₃₈₂₀ | M4 | 79 | 6477 | T | C | [symbol] | CDZ96674- |
| | | | | | | | CDZ96675 |
| X₃₈₂₁ | M4 | 79 | 57804 | G | A | [symbol] | CDZ96687 |
| X₃₈₂₂ | M4 | 79 | 64658 | C | T | [symbol] | CDZ96688 |
| X₃₈₂₃ | M4 | 79 | 65402 | C | T | [symbol] | CDZ96688 |
| X₃₈₂₄ | M4 | 79 | 132683 | C | T | [symbol] | CDZ96713 |
| X₃₈₂₅ | M4 | 79 | 147578 | G | A | [symbol] | CDZ96719 |
| X₃₈₂₆ | M4 | 79 | 255780 | G | A | [symbol] | CDZ96757 |
| X₃₈₂₇ | M4 | 79 | 281783 | C | T | [symbol] | CDZ96768 |
| X₃₈₂₈ | M4 | 79 | 307021 | C | T | [symbol] | CDZ96777 |
| X₃₈₂₉ | M4 | 79 | 345646 | G | A | [symbol] | CDZ96789 |
| X₃₈₃₀ | M4 | 79 | 346823 | G | A | [symbol] | CDZ96789 |
| X₃₈₃₁ | M4 | 79 | 368176 | G | A | [symbol] | CDZ96795 |
| X₃₈₃₂ | M4 | 79 | 406184 | G | A | [symbol] | CDZ96808 |
| X₃₈₃₃ | M4 | 79 | 412057 | T | C | [symbol] | CDZ96811 |
| X₃₈₃₄ | M4 | 79 | 412661 | G | A | [symbol] | CDZ96811 |
| X₃₈₃₅ | M4 | 79 | 492523 | C | T | [symbol] | CDZ96842 |
| X₃₈₃₆ | M4 | 79 | 526910 | C | T | [symbol] | CDZ96854 |
| X₃₈₃₇ | M4 | 79 | 552798 | C | T | [symbol] | CDZ96861 |
| X₃₈₃₈ | M4 | 79 | 573293 | C | T | [symbol] | CDZ96868 |
| X₃₈₃₉ | M4 | 79 | 575411 | C | T | [symbol] | CDZ96868 |
| X₃₈₄₀ | M4 | 79 | 579674 | G | A | [symbol] | CDZ96871 |
| X₃₈₄₁ | M4 | 79 | 611702 | C | T | [symbol] | CDZ96881 |
| X₃₈₄₂ | M4 | 79 | 617057 | C | T | [symbol] | CDZ96882 |
| X₃₈₄₃ | M4 | 79 | 679140 | C | T | [symbol] | CDZ96904 |
| X₃₈₄₄ | M4 | 79 | 696174 | G | A | [symbol] | CDZ96911 |
| X₃₈₄₅ | M4 | 79 | 735053 | T | C | [symbol] | CDZ96928 |
| X₃₈₄₆ | M4 | 79 | 748083 | G | T | [symbol] | CDZ96929 |
| X₃₈₄₇ | M4 | 79 | 758025 | G | A | [symbol] | CDZ96931 |
| X₃₈₄₈ | M4 | 79 | 759403 | C | T | [symbol] | CDZ96931 |
| X₃₈₄₉ | M4 | 79 | 759589 | G | A | [symbol] | CDZ96932 |
| X₃₈₅₀ | M4 | 79 | 759659 | G | A | [symbol] | CDZ96931 |
| X₃₈₅₁ | M4 | 79 | 760430 | T | C | [symbol] | CDZ96932 |
| X₃₈₅₂ | M4 | 79 | 760458 | G | T | [symbol] | CDZ96932 |
| X₃₈₅₃ | M4 | 79 | 760541 | A | G | [symbol] | CDZ96932 |
| X₃₈₅₄ | M4 | 79 | 760891 | A | G | [symbol] | CDZ96932 |
| X₃₈₅₅ | M4 | 79 | 760988 | G | T | [symbol] | CDZ96932 |
| X₃₈₅₆ | M4 | 79 | 761056 | T | C | [symbol] | CDZ96932 |
| X₃₈₅₇ | M4 | 79 | 761210 | T | C | [symbol] | CDZ96932 |
| X₃₈₅₈ | M4 | 79 | 768537 | A | G | [symbol] | CDZ96933 |
| X₃₈₅₉ | M4 | 79 | 786064 | A | G | [symbol] | CDZ96934 |
| X₃₈₆₀ | M4 | 79 | 798799 | C | T | [symbol] | CDZ96939 |
| X₃₈₆₁ | M4 | 79 | 830724 | C | G | [symbol] | CDZ96950 |
| X₃₈₆₂ | M4 | 79 | 874161 | G | A | [symbol] | CDZ96965 |
| X₃₈₆₃ | M4 | 79 | 907000 | C | T | [symbol] | CDZ96976 |
| X₃₈₆₄ | M4 | 79 | 908162 | G | A | [symbol] | CDZ96976 |
| X₃₈₆₅ | M4 | 79 | 914302 | C | T | [symbol] | CDZ96977 |
| X₃₈₆₆ | M4 | 79 | 920544 | C | T | [symbol] | CDZ96982 |
| X₃₈₆₇ | M4 | 79 | 922582 | G | A | [symbol] | CDZ96983 |
| X₃₈₆₈ | M4 | 79 | 933234 | G | A | [symbol] | CDZ96987 |
| X₃₈₆₉ | M4 | 79 | 940003 | C | T | [symbol] | CDZ96991 |
| X₃₉₇₀ | M4 | 79 | 962607 | G | A | [symbol] | CDZ96997 |
| X₃₈₇₁ | M4 | 79 | 973493 | C | T | [symbol] | CDZ97000 |
| X₃₈₇₂ | M4 | 79 | 1000786 | A | G | [symbol] | CDZ97007 |
| X₃₈₇₃ | M4 | 79 | 1021855 | G | A | [symbol] | CDZ97016 |
| X₃₈₇₄ | M4 | 79 | 1082233 | G | A | [symbol] | CDZ97037 |
| X₃₈₇₅ | M4 | 79 | 1107695 | C | T | [symbol] | CDZ97046 |
| X₃₈₇₆ | M4 | 79 | 1117040 | C | T | [symbol] | CDZ97047 |
| X₃₈₇₇ | M4 | 79 | 1118598 | C | T | [symbol] | CDZ97048 |
| X₃₈₇₈ | M4 | 79 | 1123288 | C | T | [symbol] | CDZ97049 |
| X₃₈₇₉ | M4 | 79 | 1140763 | G | A | [symbol] | CDZ97053 |
| X₃₈₈₀ | M4 | 79 | 1151678 | C | T | [symbol] | CDZ97060 |
| X₃₈₈₁ | M4 | 79 | 1176587 | T | A | [symbol] | CDZ97066 |
| X₃₈₈₂ | M4 | 79 | 1229129 | G | A | [symbol] | CDZ97086 |
| X₃₈₈₃ | M4 | 79 | 1231740 | C | T | [symbol] | CDZ97087 |
| X₃₈₈₄ | M4 | 79 | 1286778 | T | C | [symbol] | CDZ97106 |
| X₃₈₈₅ | M4 | 79 | 1302166 | G | A | [symbol] | CDZ97112 |
| X₃₈₈₆ | M4 | 79 | 1318034 | C | T | [symbol] | CDZ97117 |
| X₃₈₈₇ | M4 | 79 | 1336766 | C | T | [symbol] | CDZ97121 |
| X₃₈₈₈ | M4 | 79 | 1344403 | C | T | [symbol] | CDZ97124 |
| X₃₈₈₉ | M4 | 79 | 1430954 | G | A | [symbol] | CDZ97152 |
| X₃₈₉₀ | M4 | 79 | 1447480 | G | A | [symbol] | CDZ97156 |
| X₃₈₉₁ | M4 | 79 | 1472351 | C | T | [symbol] | CDZ97165 |
| X₃₈₉₂ | M4 | 79 | 1540205 | C | T | [symbol] | CDZ97186 |
| X₃₈₉₃ | M4 | 79 | 1624415 | G | A | [symbol] | CDZ97217 |
| X₃₈₉₄ | M4 | 79 | 1674448 | G | A | [symbol] | CDZ97228 |
| X₃₈₉₅ | M4 | 79 | 1677774 | C | T | [symbol] | CDZ97229 |
| X₃₈₉₆ | M4 | 79 | 1696776 | G | A | [symbol] | CDZ97234 |
| X₃₈₉₇ | M4 | 79 | 1788096 | G | A | [symbol] | CDZ97262 |
| X₃₈₉₈ | M4 | 79 | 1791482 | G | A | [symbol] | CDZ97263 |
| X₃₈₉₉ | M4 | 79 | 1797660 | C | T | [symbol] | CDZ97265 |
| X₃₉₀₀ | M4 | 79 | 1797676 | G | A | [symbol] | CDZ97265 |
| X₃₉₀₁ | M4 | 79 | 1819746 | C | T | [symbol] | CDZ97273 |
| X₃₉₀₂ | M4 | 79 | 1842448 | G | A | [symbol] | CDZ97282 |
| X₃₉₀₃ | M4 | 79 | 1855037 | A | G | [symbol] | CDZ97285 |
| X₃₉₀₄ | M4 | 79 | 1859220 | A | G | [symbol] | CDZ97291 |
| X₃₉₀₅ | M4 | 79 | 1877329 | G | A | [symbol] | CDZ97295 |
| X₃₉₀₆ | M4 | 79 | 1883004 | A | G | [symbol] | CDZ97298 |
| X₃₉₀₇ | M4 | 79 | 1883021 | C | A | [symbol] | CDZ97298 |
| X₃₉₀₈ | M4 | 79 | 1883115 | T | C | [symbol] | CDZ97298 |
| X₃₉₀₉ | M4 | 79 | 1912360 | G | A | [symbol] | CDZ97307 |
| X₃₉₁₀ | M4 | 79 | 1934434 | G | A | [symbol] | CDZ97316 |
| X₃₉₁₁ | M4 | 79 | 2013304 | G | A | [symbol] | CDZ97344 |
| X₃₉₁₂ | M4 | 79 | 2013361 | C | T | [symbol] | CDZ97344 |
| X₃₉₁₃ | M4 | 79 | 2115870 | G | A | [symbol] | CDZ97380 |
| X₃₉₁₄ | M4 | 79 | 2121041 | G | A | [symbol] | CDZ97380 |
| X₃₉₁₅ | M4 | 79 | 2152573 | C | T | [symbol] | CDZ97392 |
| X₃₉₁₆ | M4 | 79 | 2182096 | G | A | [symbol] | CDZ97403 |
| X₃₉₁₇ | M4 | 79 | 2188410 | G | A | [symbol] | CDZ97405 |
| X₃₉₁₈ | M4 | 79 | 2220254 | C | T | [symbol] | CDZ97414 |
| X₃₉₁₉ | M4 | 79 | 2268540 | C | T | [symbol] | CDZ97435 |
| X₃₉₂₀ | M4 | 79 | 2315869 | A | C | [symbol] | CDZ97452 |
| X₃₉₂₁ | M4 | 79 | 2339881 | C | G | [symbol] | CDZ97460 |
| X₃₉₂₂ | M4 | 79 | 2340326 | G | A | [symbol] | CDZ97462 |
| X₃₉₂₃ | M4 | 79 | 2340636 | T | C | [symbol] | CDZ97462 |
| X₃₉₂₄ | M4 | 85 | 512 | A | G | [symbol] | |
| X₃₉₂₅ | M4 | 85 | 516 | G | A | [symbol] | |
| X₃₉₂₆ | M4 | 85 | 521 | C | T | [symbol] | |
| X₃₉₂₇ | M4 | 85 | 522 | A | G | [symbol] | |
| X₃₉₂₈ | M4 | 88 | 119 | C | T | [symbol] | CDZ97464 |
| X₃₉₂₉ | M4 | 88 | 196 | G | A | [symbol] | CDZ97464 |
| X₃₉₃₀ | M4 | 88 | 898 | C | T | [symbol] | CDZ97464 |
| X₃₉₃₁ | M4 | 99 | 245 | G | A | [symbol] | |
| X₃₉₃₂ | M4 | 105 | 258 | A | G | [symbol] | CDZ97474 |
| X₃₉₃₃ | M4 | 105 | 429 | C | T | [symbol] | CDZ97474 |
| X₃₉₃₄ | M4 | 109 | 396 | T | C | [symbol] | |
| X₃₉₃₅ | M4 | 118 | 396 | C | T | [symbol] | CDZ97478 |
| X₃₉₃₆ | M4 | 118 | 870 | G | A | [symbol] | CDZ97478 |
| X₃₉₃₇ | M4 | 118 | 1491 | G | A | [symbol] | CDZ97478 |
| X₃₉₃₈ | M4 | 121 | 452 | G | A | [symbol] | |
| X₃₉₃₉ | M4 | 121 | 907 | T | C | [symbol] | |
| X₃₉₄₀ | M4 | 124 | 65147 | C | T | [symbol] | CDZ97499 |
| X₃₉₄₁ | M4 | 124 | 72117 | G | A | [symbol] | CDZ97501 |
| X₃₉₄₂ | M4 | 124 | 72760 | G | A | [symbol] | CDZ97501 |
| X₃₉₄₃ | M4 | 124 | 76965 | A | T | [symbol] | CDZ97503 |
| X₃₉₄₄ | M4 | 124 | 94611 | G | A | [symbol] | CDZ97507 |
| X₃₉₄₅ | M4 | 124 | 111749 | G | A | [symbol] | CDZ97512 |
| X₃₉₄₆ | M4 | 124 | 156052 | G | A | [symbol] | CDZ97524 |
| X₃₉₄₇ | M4 | 124 | 166988 | C | T | [symbol] | CDZ97527 |
| X₃₉₄₈ | M4 | 124 | 206541 | G | A | [symbol] | CDZ97541 |
| X₃₉₄₉ | M4 | 124 | 207992 | C | T | [symbol] | CDZ97541 |
| X₃₉₅₀ | M4 | 124 | 222249 | C | T | [symbol] | CDZ97547 |
| X₃₉₅₁ | M4 | 125 | 16379 | G | A | [symbol] | CDZ97565 |
| X₃₉₅₂ | M4 | 125 | 35864 | T | C | [symbol] | CDZ97573 |
| X₃₉₅₃ | M4 | 127 | 1558 | T | C | [symbol] | |
| X₃₉₅₄ | M4 | 129 | 299 | G | A | [symbol] | CDZ97577 |
| X₃₉₅₅ | M4 | 129 | 433 | G | A | [symbol] | CDZ97577 |
| X₃₉₅₆ | M4 | 129 | 856 | G | A | [symbol] | CDZ97577 |
| X₃₉₅₇ | M4 | 129 | 1013 | C | T | [symbol] | CDZ97577 |
| X₃₉₅₈ | M4 | 130 | 67 | C | T | [symbol] | CDZ97578 |
| X₃₉₅₉ | M4 | 141 | 15764 | C | T | [symbol] | CDZ97587 |
| X₃₉₆₀ | M4 | 149 | 1663 | A | G | [symbol] | CDZ97598 |
| X₃₉₆₁ | M4 | 149 | 2074 | T | G | [symbol] | CDZ97598 |
| X₃₉₆₂ | M4 | 149 | 2453 | C | T | [symbol] | CDZ97598 |
| X₃₉₆₃ | M4 | 159 | 106 | A | C | [symbol] | |
| X₃₉₆₄ | M4 | 162 | 47699 | G | A | [symbol] | CDZ97619 |
| X₃₉₆₅ | M4 | 162 | 68192 | C | G | [symbol] | CDZ97625 |
| X₃₉₆₆ | M4 | 162 | 70405 | A | G | [symbol] | CDZ97625 |
| X₃₉₆₇ | M4 | 162 | 91190 | C | T | [symbol] | CDZ97631 |
| X₃₉₆₈ | M4 | 162 | 96194 | G | A | [symbol] | CDZ97631 |
| X₃₉₆₉ | M4 | 162 | 112967 | C | T | [symbol] | CDZ97638 |
| X₃₉₇₀ | M4 | 162 | 117736 | G | A | [symbol] | CDZ97639 |
| X₃₉₇₁ | M4 | 162 | 223407 | C | T | [symbol] | CDZ97674 |
| X₃₉₇₂ | M4 | 162 | 231146 | C | T | [symbol] | CDZ97676 |
| X₃₉₇₃ | M4 | 162 | 233092 | C | T | [symbol] | CDZ97676 |
| X₃₉₇₄ | M4 | 162 | 238126 | C | T | [symbol] | CDZ97680 |
| X₃₉₇₅ | M4 | 162 | 243753 | G | A | [symbol] | CDZ97680 |
| X₃₉₇₆ | M4 | 162 | 308369 | T | C | [symbol] | CDZ97702 |
| X₃₉₇₇ | M4 | 162 | 320701 | G | A | [symbol] | CDZ97703 |
| X₃₉₇₈ | M4 | 162 | 321460 | C | G | [symbol] | CDZ97703 |
| X₃₉₇₉ | M4 | 162 | 321463 | A | C | [symbol] | CDZ97703 |
| X₃₉₈₀ | M4 | 162 | 322591 | G | A | [symbol] | CDZ97703 |
| X₃₉₈₁ | M4 | 162 | 323448 | T | G | [symbol] | CDZ97703 |
| X₃₉₈₂ | M4 | 162 | 323451 | T | G | [symbol] | CDZ97703 |
| X₃₉₈₃ | M4 | 162 | 324114 | T | C | [symbol] | CDZ97703 |
| X₃₉₈₄ | M4 | 162 | 324139 | C | T | [symbol] | CDZ97703 |
| X₃₉₈₅ | M4 | 162 | 324568 | A | G | [symbol] | CDZ97703 |
| X₃₉₈₆ | M4 | 162 | 351016 | C | T | [symbol] | CDZ97711 |
| X₃₉₈₇ | M4 | 162 | 360320 | G | A | [symbol] | CDZ97714 |
| X₃₉₈₈ | M4 | 162 | 398165 | C | T | [symbol] | CDZ97730 |
| X₃₉₈₉ | M4 | 162 | 408274 | C | T | [symbol] | CDZ97733 |
| X₃₉₉₀ | M4 | 162 | 424391 | C | T | [symbol] | CDZ97740 |
| X₃₉₉₁ | M4 | 162 | 444123 | T | C | [symbol] | CDZ97748 |
| X₃₉₉₂ | M4 | 162 | 453186 | G | A | [symbol] | CDZ97750 |
| X₃₉₉₃ | M4 | 162 | 456498 | C | T | [symbol] | CDZ97752 |
| X₃₉₉₄ | M4 | 162 | 467207 | C | T | [symbol] | CDZ97755 |
| X₃₉₉₅ | M4 | 162 | 476267 | C | T | [symbol] | CDZ97760 |
| X₃₉₉₆ | M4 | 162 | 480722 | G | A | [symbol] | CDZ97761 |
| X₃₉₉₇ | M4 | 162 | 491906 | G | A | [symbol] | CDZ97764 |
| X₃₉₉₈ | M4 | 162 | 492101 | G | A | [symbol] | CDZ97764 |
| X₃₉₉₉ | M4 | 162 | 501886 | C | T | [symbol] | CDZ97769 |
| X₄₀₀₀ | M4 | 162 | 618658 | C | T | [symbol] | CDZ97808 |
| X₄₀₀₁ | M4 | 162 | 631700 | G | A | [symbol] | CDZ97811 |
| X₄₀₀₂ | M4 | 162 | 632311 | G | A | [symbol] | CDZ97814 |
| X₄₀₀₃ | M4 | 162 | 633321 | G | A | [symbol] | CDZ97814 |
| X₄₀₀₄ | M4 | 162 | 633633 | G | A | [symbol] | CDZ97814 |
| X₄₀₀₅ | M4 | 162 | 640005 | C | T | [symbol] | CDZ97815 |
| X₄₀₀₆ | M4 | 162 | 645194 | G | A | [symbol] | CDZ97815 |
| X₄₀₀₇ | M4 | 162 | 690472 | C | T | [symbol] | CDZ97827 |
| X₄₀₀₈ | M4 | 162 | 738479 | G | A | [symbol] | CDZ97842 |
| X₄₀₀₉ | M4 | 162 | 738675 | G | A | [symbol] | CDZ97842 |
| X₄₀₁₀ | M4 | 162 | 738686 | C | T | [symbol] | CDZ97842 |
| X₄₀₁₁ | M4 | 162 | 808830 | G | A | [symbol] | CDZ97869 |
| X₄₀₁₂ | M4 | 162 | 825209 | G | A | [symbol] | CDZ97877 |
| X₄₀₁₃ | M4 | 162 | 841825 | A | G | [symbol] | CDZ97882 |
| X₄₀₁₄ | M4 | 162 | 871911 | C | T | [symbol] | CDZ97890 |
| X₄₀₁₅ | M4 | 162 | 967098 | A | G | [symbol] | CDZ97922 |
| X₄₀₁₆ | M4 | 162 | 972973 | G | A | [symbol] | CDZ97925 |
| X₄₀₁₇ | M4 | 163 | 727 | A | G | [symbol] | |
| X₄₀₁₈ | M4 | 169 | 1056 | C | T | [symbol] | |
| X₄₀₁₉ | M4 | 189 | 12794 | C | T | [symbol] | CDZ97963 |
| X₄₀₂₀ | M4 | 189 | 126573 | G | A | [symbol] | CDZ97996 |
| X₄₀₂₁ | M4 | 189 | 127854 | C | T | [symbol] | CDZ97998 |
| X₄₀₂₂ | M4 | 189 | 194647 | A | G | [symbol] | CDZ98020 |
| X₄₀₂₃ | M4 | 189 | 194665 | A | G | [symbol] | CDZ98020 |
| X₄₀₂₄ | M4 | 189 | 211865 | C | T | [symbol] | CDZ98028 |
| X₄₀₂₅ | M4 | 189 | 260617 | A | T | [symbol] | CDZ98041 |
| X₄₀₂₆ | M4 | 189 | 300343 | C | T | [symbol] | CDZ98056 |
| X₄₀₂₇ | M4 | 189 | 303106 | G | A | [symbol] | CDZ98057 |
| X₄₀₂₈ | M4 | 189 | 323680 | C | T | [symbol] | CDZ98063 |
| X₄₀₂₉ | M4 | 189 | 344057 | C | T | [symbol] | CDZ98068 |
| X₄₀₃₀ | M4 | 189 | 371247 | G | A | [symbol] | CDZ98078 |
| X₄₀₃₁ | M4 | 189 | 452298 | G | A | [symbol] | CDZ98106 |
| X₄₀₃₂ | M4 | 191 | 342 | A | G | [symbol] | CDZ98107 |
| X₄₀₃₃ | M4 | 191 | 347 | C | T | [symbol] | CDZ98107 |
| X₄₀₃₄ | M4 | 191 | 427 | C | T | [symbol] | CDZ98107 |
| X₄₀₃₅ | M4 | 191 | 451 | C | T | [symbol] | CDZ98107 |
| X₄₀₃₆ | M4 | 191 | 487 | A | G | [symbol] | CDZ98107 |
| X₄₀₃₇ | M4 | 197 | 624 | C | T | [symbol] | CDZ98111 |
| X₄₀₃₈ | M4 | 198 | 202 | C | A | [symbol] | |
| X₄₀₃₉ | M4 | 198 | 203 | G | A | [symbol] | |
| X₄₀₄₀ | M4 | 198 | 238 | C | T | [symbol] | |
| X₄₀₄₁ | M4 | 198 | 265 | C | T | [symbol] | |
| X₄₀₄₂ | M4 | 198 | 452 | G | A | [symbol] | |
| X₄₀₄₃ | M4 | 199 | 688 | C | T | [symbol] | |
| X₄₀₄₄ | M4 | 199 | 911 | C | T | [symbol] | |
| X₄₀₄₅ | M4 | 199 | 971 | C | T | [symbol] | |
| X₄₀₄₆ | M4 | 199 | 1218 | C | T | [symbol] | |
| X₄₀₄₇ | M4 | 202 | 171 | C | T | [symbol] | |
| X₄₀₄₈ | M4 | 205 | 360 | C | T | [symbol] | CDZ98112 |
| X₄₀₄₉ | M4 | 205 | 981 | C | T | [symbol] | CDZ98112 |
| X₄₀₅₀ | M4 | 206 | 406 | G | A | [symbol] | |
| X₄₀₅₁ | M4 | 208 | 117 | G | A | [symbol] | |
| X₄₀₅₂ | M4 | 208 | 476 | G | A | [symbol] | |
| X₄₀₅₃ | M4 | 208 | 616 | G | A | [symbol] | |
| X₄₀₅₄ | M4 | 208 | 935 | G | A | [symbol] | |
| X₄₀₅₅ | M4 | 209 | 312 | C | T | [symbol] | CDZ98113 |
| X₄₀₅₆ | M4 | 214 | 197 | C | G | [symbol] | CDZ98116 |
| X₄₀₅₇ | M4 | 220 | 116 | C | G | [symbol] | |
| X₄₀₅₈ | M4 | 223 | 93 | G | A | [symbol] | CDZ98118 |
| X₄₀₅₉ | M4 | 223 | 102 | A | G | [symbol] | CDZ98118 |
| X₄₀₆₀ | M4 | 223 | 133 | A | G | [symbol] | CDZ98118 |
| X₄₀₆₁ | M4 | 223 | 150 | T | C | [symbol] | CDZ98118 |
| X₄₀₆₂ | M4 | 223 | 155 | A | C | [symbol] | CDZ98118 |
| X₄₀₆₃ | M4 | 223 | 158 | T | C | [symbol] | CDZ98118 |
| X₄₀₆₄ | M4 | 223 | 160 | C | T | [symbol] | CDZ98118 |
| X₄₀₆₅ | M4 | 223 | 175 | G | A | [symbol] | CDZ98118 |
| X₄₀₆₆ | M4 | 223 | 177 | A | G | [symbol] | CDZ98118 |
| X₄₀₆₇ | M4 | 223 | 188 | A | G | [symbol] | CDZ98118 |
| X₄₀₆₈ | M4 | 223 | 192 | C | A | [symbol] | CDZ98118 |
| X₄₀₆₉ | M4 | 223 | 194 | T | C | [symbol] | CDZ98118 |
| X₄₀₇₀ | M4 | 223 | 195 | T | G | [symbol] | CDZ98118 |
| X₄₀₇₁ | M4 | 223 | 197 | T | C | [symbol] | CDZ98118 |
| X₄₀₇₂ | M4 | 223 | 202 | G | C | [symbol] | CDZ98118 |
| X₄₀₇₃ | M4 | 223 | 205 | C | T | [symbol] | CDZ98118 |
| X₄₀₇₄ | M4 | 224 | 27 | T | A | [symbol] | |
| X₄₀₇₅ | M4 | 225 | 317 | C | T | [symbol] | |
| X₄₀₇₆ | M4 | 227 | 202 | G | A | [symbol] | |
| X₄₀₇₇ | M4 | 232 | 340 | G | A | [symbol] | CDZ98119 |
| X₄₀₇₈ | M4 | 232 | 1379 | C | T | [symbol] | CDZ98119 |
| X₄₀₇₉ | M4 | 232 | 1521 | C | T | [symbol] | CDZ98119 |
| X₄₀₈₀ | M4 | 232 | 1662 | G | A | [symbol] | CDZ98119 |
| X₄₀₈₁ | M4 | 232 | 1761 | C | T | [symbol] | CDZ98119 |
| X₄₀₈₂ | M4 | 232 | 2027 | C | T | [symbol] | CDZ98119 |
| X₄₀₈₃ | M4 | 232 | 3060 | C | T | [symbol] | CDZ98120 |
| X₄₀₈₄ | M4 | 232 | 3284 | C | T | [symbol] | CDZ98119 |
| X₄₀₈₅ | M4 | 232 | 4437 | G | A | [symbol] | CDZ98121 |
| X₄₀₈₆ | M4 | 232 | 4561 | C | T | [symbol] | CDZ98120 |
| X₄₀₈₇ | M4 | 242 | 7334 | C | T | [symbol] | CDZ98125 |
| X₄₀₈₈ | M4 | 242 | 41710 | G | A | [symbol] | CDZ98131 |
| X₄₀₈₉ | M4 | 242 | 50262 | G | A | [symbol] | CDZ98135 |
| X₄₀₉₀ | M4 | 242 | 107587 | G | A | [symbol] | CDZ98153 |
| X₄₀₉₁ | M4 | 242 | 213399 | C | T | [symbol] | CDZ98185 |
| X₄₀₉₂ | M4 | 242 | 213986 | G | A | [symbol] | CDZ98185 |
| X₄₀₉₃ | M4 | 242 | 227271 | C | T | [symbol] | CDZ98191 |
| X₄₀₉₄ | M4 | 242 | 233088 | G | A | [symbol] | CDZ98193 |
| X₄₀₉₅ | M4 | 242 | 310353 | G | A | [symbol] | CDZ98216 |
| X₄₀₉₆ | M4 | 242 | 357209 | G | A | [symbol] | CDZ98233 |
| X₄₀₉₇ | M4 | 242 | 404132 | G | A | [symbol] | CDZ98248 |
| X₄₀₉₈ | M4 | 242 | 412676 | G | A | [symbol] | CDZ98249 |
| X₄₀₉₉ | M4 | 242 | 437941 | G | A | [symbol] | CDZ98258 |
| X₄₁₀₀ | M4 | 242 | 454709 | C | T | [symbol] | CDZ98263 |
| X₄₁₀₁ | M4 | 242 | 485936 | G | A | [symbol] | CDZ98270 |
| X₄₁₀₂ | M4 | 242 | 492389 | G | A | [symbol] | CDZ98274 |
| X₄₁₀₃ | M4 | 242 | 514780 | C | T | [symbol] | CDZ98282 |
| X₄₁₀₄ | M4 | 242 | 528340 | G | A | [symbol] | CDZ98288 |
| X₄₁₀₅ | M4 | 242 | 542663 | G | A | [symbol] | CDZ98290 |
| X₄₁₀₆ | M4 | 242 | 559999 | G | A | [symbol] | CDZ98297 |
| X₄₁₀₇ | M4 | 242 | 577367 | G | A | [symbol] | CDZ98302 |
| X₄₁₀₈ | M4 | 242 | 602456 | G | A | [symbol] | CDZ98308 |
| X₄₁₀₉ | M4 | 242 | 602531 | G | A | [symbol] | CDZ98308 |
| X₄₁₁₀ | M4 | 242 | 604704 | C | T | [symbol] | CDZ98309 |
| X₄₁₁₁ | M4 | 242 | 641669 | G | A | [symbol] | CDZ98323 |
| X₄₁₁₂ | M4 | 248 | 99 | A | G | [symbol] | |
| X₄₁₁₃ | M4 | 249 | 42295 | C | T | [symbol] | CED84942 |
| X₄₁₁₄ | M4 | 249 | 72016 | C | T | [symbol] | CED84950 |
| X₄₁₁₅ | M4 | 249 | 157550 | C | T | [symbol] | CED84983 |
| X₄₁₁₆ | M4 | 249 | 191304 | C | T | [symbol] | CED84988 |
| X₄₁₁₇ | M4 | 249 | 253494 | C | T | [symbol] | CED85008 |
| X₄₁₁₈ | M4 | 249 | 272075 | C | T | [symbol] | CED85013 |
| X₄₁₁₉ | M4 | 249 | 305975 | G | A | [symbol] | CED85023 |
| X₄₁₂₀ | M4 | 249 | 311154 | G | A | [symbol] | CED85025 |
| X₄₁₂₁ | M4 | 249 | 342990 | G | A | [symbol] | CED85036 |
| X₄₁₂₂ | M4 | 249 | 370636 | T | C | [symbol] | CED85048 |
| X₄₁₂₃ | M4 | 249 | 371535 | G | A | [symbol] | CED85048 |
| X₄₁₂₄ | M4 | 249 | 377814 | G | A | [symbol] | CED85051 |
| X₄₁₂₅ | M4 | 249 | 445429 | G | A | [symbol] | CED85075 |
| X₄₁₂₆ | M4 | 249 | 469645 | G | A | [symbol] | CED85087 |
| X₄₁₂₇ | M4 | 249 | 485782 | C | T | [symbol] | CED85089 |
| X₄₁₂₈ | M4 | 249 | 501310 | C | T | [symbol] | CED85093 |
| X₄₁₂₉ | M4 | 249 | 501510 | T | C | [symbol] | CED85093 |
| X₄₁₃₀ | M4 | 249 | 506067 | A | G | [symbol] | CED85094 |
| X₄₁₃₁ | M4 | 249 | 532943 | A | G | [symbol] | CED85099 |
| X₄₁₃₂ | M4 | 249 | 571922 | C | T | [symbol] | CED85115 |
| X₄₁₃₃ | M4 | 249 | 576070 | G | A | [symbol] | CED85118 |
| X₄₁₃₄ | M4 | 249 | 598579 | C | T | [symbol] | CED85125 |
| X₄₁₃₅ | M4 | 249 | 627104 | C | T | [symbol] | CED85133 |
| X₄₁₃₆ | M4 | 249 | 637944 | G | A | [symbol] | CED85135 |
| X₄₁₃₇ | M4 | 249 | 660408 | G | A | [symbol] | CED85144 |
| X₄₁₃₈ | M4 | 249 | 709267 | C | T | [symbol] | CED85162 |
| X₄₁₃₉ | M4 | 249 | 746655 | C | T | [symbol] | CED85172 |
| X₄₁₄₀ | M4 | 249 | 854220 | G | A | [symbol] | CED85201 |
| X₄₁₄₁ | M4 | 249 | 886063 | G | A | [symbol] | CED85208 |
| X₄₁₄₂ | M4 | 249 | 911274 | C | T | [symbol] | CED85218 |
| X₄₁₄₃ | M4 | 249 | 932606 | C | T | [symbol] | CED85223 |
| X₄₁₄₄ | M4 | 249 | 945434 | C | T | [symbol] | CED85227 |
| X₄₁₄₅ | M4 | 249 | 945727 | C | T | [symbol] | CED85227 |
| X₄₁₄₆ | M4 | 249 | 969333 | G | T | [symbol] | CED85238 |
| X₄₁₄₇ | M4 | 249 | 972975 | G | A | [symbol] | CED85240 |
| X₄₁₄₈ | M4 | 249 | 977170 | C | T | [symbol] | CED85242 |
| X₄₁₄₉ | M4 | 249 | 989520 | C | T | [symbol] | CED85243 |
| X₄₁₅₀ | M4 | 249 | 1003476 | G | A | [symbol] | CED85249 |
| X₄₁₅₁ | M4 | 249 | 1014098 | C | T | [symbol] | CED85252 |
| X₄₁₅₂ | M4 | 249 | 1062282 | G | A | [symbol] | CED85266 |
| X₄₁₅₃ | M4 | 249 | 1064359 | C | T | [symbol] | CED85265 |
| X₄₁₅₄ | M4 | 249 | 1076333 | G | A | [symbol] | CED85270 |
| X₄₁₅₅ | M4 | 249 | 1088841 | T | C | [symbol] | CED85274 |
| X₄₁₅₆ | M4 | 249 | 1127638 | C | T | [symbol] | CED85286 |
| X₄₁₅₇ | M4 | 249 | 1143228 | C | T | [symbol] | CED85292 |
| X₄₁₅₈ | M4 | 249 | 1144309 | G | A | [symbol] | CED85291 |
| X₄₁₅₉ | M4 | 249 | 1176977 | G | A | [symbol] | CED85303 |
| X₄₁₆₀ | M4 | 24 | 1195367 | G | A | [symbol] | CED85307 |
| X₄₁₆₁ | M4 | 24 | 1223330 | C | T | [symbol] | CED85317 |
| X₄₁₆₂ | M4 | 249 | 1230068 | C | T | [symbol] | CED85319 |
| X₄₁₆₃ | M4 | 249 | 1279596 | C | T | [symbol] | CED85335 |
| X₄₁₆₄ | M4 | 249 | 1299472 | C | T | [symbol] | CED85343 |
| X₄₁₆₅ | M4 | 249 | 1498029 | T | C | [symbol] | CED85415 |
| X₄₁₆₆ | M4 | 249 | 1498823 | T | C | [symbol] | CED85414 |
| X₄₁₆₇ | M4 | 249 | 1498829 | C | T | [symbol] | CED85414 |
| X₄₁₆₈ | M4 | 249 | 1499004 | T | C | [symbol] | CED85415 |
| X₄₁₆₉ | M4 | 249 | 1499008 | A | G | [symbol] | CED85415 |
| X₄₁₇₀ | M4 | 249 | 1502631 | G | T | [symbol] | CED85416 |
| X₄₁₇₁ | M4 | 249 | 1502641 | T | A | [symbol] | CED85416 |
| X₄₁₇₂ | M4 | 249 | 1502819 | T | G | [symbol] | CED85416 |
| X₄₁₇₃ | M4 | 249 | 1505138 | G | A | [symbol] | CED85416 |
| X₄₁₇₄ | M4 | 249 | 1518591 | T | A | [symbol] | CED85419 |
| X₄₁₇₅ | M4 | 249 | 1519431 | A | G | [symbol] | CED85419 |
| X₄₁₇₆ | M4 | 249 | 1535511 | G | A | [symbol] | CED85423 |
| X₄₁₇₇ | M4 | 249 | 1619036 | G | A | [symbol] | CED85450 |
| X₄₁₇₈ | M4 | 249 | 1621516 | G | A | [symbol] | CED85451 |
| X₄₁₇₉ | M4 | 249 | 1623151 | G | A | [symbol] | CED85451 |
| X₄₁₈₀ | M4 | 249 | 1653615 | T | G | [symbol] | CED85457 |
| X₄₁₈₁ | M4 | 249 | 1653962 | C | T | [symbol] | CED85458 |
| X₄₁₈₂ | M4 | 249 | 1666185 | C | T | [symbol] | CED85460 |
| X₄₁₈₃ | M4 | 249 | 1691321 | G | A | [symbol] | CED85467 |
| X₄₁₈₄ | M4 | 249 | 1726777 | C | T | [symbol] | CED85474 |
| X₄₁₈₅ | M4 | 249 | 1730645 | C | T | [symbol] | CED85477 |
| X₄₁₈₆ | M4 | 249 | 1734722 | G | A | [symbol] | CED85477 |
| X₄₁₈₇ | M4 | 249 | 1741408 | C | T | [symbol] | CED85480 |
| X₄₁₈₈ | M4 | 249 | 1780551 | A | G | [symbol] | CED85493 |
| X₄₁₈₉ | M4 | 249 | 1780569 | A | G | [symbol] | CED85493 |
| X₄₁₉₀ | M4 | 249 | 1801965 | G | A | [symbol] | CED85501 |
| X₄₁₉₁ | M4 | 249 | 1803575 | C | T | [symbol] | CED85502 |
| X₄₁₉₂ | M4 | 249 | 1803790 | G | A | [symbol] | CED85502 |
| X₄₁₉₃ | M4 | 249 | 1880762 | G | A | [symbol] | CED85525 |
| X₄₁₉₄ | M4 | 249 | 1915336 | C | T | [symbol] | CED85537 |
| X₄₁₉₅ | M4 | 249 | 1944026 | G | A | [symbol] | CED85545 |
| X₄₁₉₆ | M4 | 249 | 1977554 | G | A | [symbol] | CED85556 |
| X₄₁₉₇ | M4 | 249 | 1981230 | G | A | [symbol] | CED85557 |
| X₄₁₉₈ | M4 | 249 | 1988505 | C | T | [symbol] | CED85560 |
| X₄₁₉₉ | M4 | 249 | 2010712 | G | A | [symbol] | CED85564 |
| X₄₂₀₀ | M4 | 249 | 2015272 | G | A | [symbol] | CED85567 |
| X₄₂₀₁ | M4 | 249 | 2027477 | G | A | [symbol] | CED85573 |
| X₄₂₀₂ | M4 | 249 | 2063221 | G | A | [symbol] | CED85586 |
| X₄₂₀₃ | M4 | 249 | 2110956 | C | T | [symbol] | CED85603 |
| X₄₂₀₄ | M4 | 249 | 2124517 | C | T | [symbol] | CED85607 |
| X₄₂₀₅ | M4 | 249 | 2178506 | G | A | [symbol] | CED85622 |
| X₄₂₀₆ | M4 | 249 | 2198205 | G | A | [symbol] | CED85628 |
| X₄₂₀₇ | M4 | 249 | 2198412 | G | A | [symbol] | CED85628 |
| X₄₂₀₈ | M4 | 249 | 2223626 | G | A | [symbol] | CED85633 |
| X₄₂₀₉ | M4 | 249 | 2254072 | G | A | [symbol] | CED85640 |
| X₄₂₁₀ | M4 | 249 | 2255547 | G | C | [symbol] | CED85640 |
| X₄₂₁₁ | M4 | 251 | 1089 | T | C | [symbol] | |
| X₄₂₁₂ | M4 | 251 | 1135 | G | A | [symbol] | |
| X₄₂₁₃ | M4 | 252 | 512 | C | T | [symbol] | |
| X₄₂₁₄ | M4 | 252 | 524 | C | T | [symbol] | |
| X₄₂₁₅ | M4 | 258 | 1139 | A | T | [symbol] | |
| X₄₂₁₆ | M4 | 260 | 156 | G | A | [symbol] | |
| X₄₂₁₇ | M4 | 262 | 4049 | G | A | [symbol] | CDZ98330 |
| X₄₂₁₈ | M4 | 262 | 16863 | C | T | [symbol] | CDZ98334 |
| X₄₂₁₉ | M4 | 262 | 30347 | G | A | [symbol] | CDZ98338 |
| X₄₂₂₀ | M4 | 262 | 30359 | G | A | [symbol] | CDZ98338 |
| X₄₂₂₁ | M4 | 262 | 81226 | G | A | [symbol] | CDZ98358 |
| X₄₂₂₂ | M4 | 262 | 108865 | C | T | [symbol] | CDZ98369 |
| X₄₂₂₃ | M4 | 262 | 109833 | C | T | [symbol] | CDZ98369 |
| X₄₂₂₄ | M4 | 262 | 134217 | G | A | [symbol] | CDZ98371 |
| X₄₂₂₅ | M4 | 262 | 148151 | C | T | [symbol] | CDZ98375 |
| X₄₂₂₆ | M4 | 262 | 164438 | G | A | [symbol] | CDZ98383 |
| X₄₂₂₇ | M4 | 262 | 175142 | G | A | [symbol] | CDZ98388 |
| X₄₂₂₈ | M4 | 262 | 201531 | G | A | [symbol] | CDZ98396 |
| X₄₂₂₉ | M4 | 262 | 221937 | C | T | [symbol] | CDZ98403 |
| X₄₂₃₀ | M4 | 262 | 286191 | C | T | [symbol] | CDZ98425 |
| X₄₂₃₁ | M4 | 262 | 286196 | T | A | [symbol] | CDZ98425 |
| X₄₂₃₂ | M4 | 262 | 319302 | G | A | [symbol] | CDZ98436 |
| X₄₂₃₃ | M4 | 262 | 380519 | C | T | [symbol] | CDZ98457 |
| X₄₂₃₄ | M4 | 262 | 383215 | C | T | [symbol] | CDZ98459 |
| X₄₂₃₅ | M4 | 262 | 392930 | C | T | [symbol] | CDZ98461 |
| X₄₂₃₆ | M4 | 262 | 397539 | G | A | [symbol] | CDZ98463 |
| X₄₂₃₇ | M4 | 262 | 414403 | C | T | [symbol] | CDZ98467 |
| X₄₂₃₈ | M4 | 262 | 420102 | G | A | [symbol] | CDZ98468 |
| X₄₂₃₉ | M4 | 262 | 444331 | G | A | [symbol] | CDZ98477 |
| X₄₂₄₀ | M4 | 262 | 446956 | C | T | [symbol] | CDZ98477 |
| X₄₂₄₁ | M4 | 262 | 539428 | G | A | [symbol] | CDZ98514 |
| X₄₂₄₂ | M4 | 262 | 539452 | A | G | [symbol] | CDZ98514 |
| X₄₂₄₃ | M4 | 262 | 539470 | A | G | [symbol] | CDZ98514 |
| X₄₂₄₄ | M4 | 262 | 557280 | G | A | [symbol] | CDZ98518 |
| X₄₂₄₅ | M4 | 262 | 560713 | G | A | [symbol] | CDZ98519 |
| X₄₂₄₆ | M4 | 262 | 562795 | C | T | [symbol] | CDZ98521 |
| X₄₂₄₇ | M4 | 262 | 567591 | G | A | [symbol] | CDZ98524 |
| X₄₂₄₈ | M4 | 262 | 567801 | G | A | [symbol] | CDZ98524 |
| X₄₂₄₉ | M4 | 262 | 573667 | C | T | [symbol] | CDZ98526 |
| X₄₂₅₀ | M4 | 262 | 618198 | G | A | [symbol] | CDZ98538 |
| X₄₂₅₁ | M4 | 262 | 638804 | G | C | [symbol] | CDZ98544 |
| X₄₂₅₂ | M4 | 262 | 639869 | G | C | [symbol] | CDZ98544 |
| X₄₂₅₃ | M4 | 262 | 639962 | A | G | [symbol] | CDZ98544 |
| X₄₂₅₄ | M4 | 262 | 653920 | A | G | [symbol] | CDZ98545 |
| X₄₂₅₅ | M4 | 262 | 655754 | A | T | [symbol] | CDZ98545 |
| X₄₂₅₆ | M4 | 262 | 655757 | T | C | [symbol] | CDZ98545 |
| X₄₂₅₇ | M4 | 262 | 656031 | C | A | [symbol] | CDZ98545 |
| X₄₂₅₈ | M4 | 262 | 668764 | C | T | [symbol] | CDZ98545 |
| X₄₂₅₉ | M4 | 262 | 673556 | G | A | [symbol] | CDZ98548 |
| X₄₂₆₀ | M4 | 262 | 691577 | C | T | [symbol] | CDZ98551 |
| X₄₂₆₁ | M4 | 262 | 822734 | G | A | [symbol] | CDZ98597 |
| X₄₂₆₂ | M4 | 262 | 835874 | C | T | [symbol] | CDZ98601 |
| X₄₂₆₃ | M4 | 262 | 865999 | G | A | [symbol] | CDZ98613 |
| X₄₂₆₄ | M4 | 262 | 969192 | G | A | [symbol] | CDZ98649 |
| X₄₂₆₅ | M4 | 262 | 971713 | G | A | [symbol] | CDZ98649 |
| X₄₂₆₆ | M4 | 262 | 1011054 | G | A | [symbol] | CDZ98659 |
| X₄₂₆₇ | M4 | 262 | 1051389 | C | T | [symbol] | CDZ98670 |
| X₄₂₆₈ | M4 | 262 | 1060326 | C | T | [symbol] | CDZ98672 |
| X₄₂₆₉ | M4 | 262 | 1076581 | G | A | [symbol] | CDZ98679 |
| X₄₂₇₀ | M4 | 262 | 1140198 | C | T | [symbol] | CDZ98701 |
| X₄₂₇₁ | M4 | 262 | 1169777 | G | A | [symbol] | CDZ98710 |
| X₄₂₇₂ | M4 | 262 | 1209662 | G | A | [symbol] | CDZ98723 |
| X₄₂₇₃ | M4 | 262 | 1226072 | C | T | [symbol] | CDZ98730 |
| X₄₂₇₄ | M4 | 262 | 1248058 | C | T | [symbol] | CDZ98737 |
| X₄₂₇₅ | M4 | 262 | 1257961 | C | T | [symbol] | CDZ98739 |
| X₄₂₇₆ | M4 | 262 | 1277604 | G | A | [symbol] | CDZ98747 |
| X₄₂₇₇ | M4 | 262 | 1305361 | G | A | [symbol] | CDZ98753 |
| X₄₂₇₈ | M4 | 262 | 1329643 | C | T | [symbol] | CDZ98760 |
| X₄₂₇₉ | M4 | 262 | 1350321 | G | A | [symbol] | CDZ98766 |
| X₄₂₈₀ | M4 | 262 | 1351116 | A | G | [symbol] | CDZ98766 |
| X₄₂₈₁ | M4 | 262 | 1415479 | C | T | [symbol] | CDZ98789 |
| X₄₂₈₂ | M4 | 262 | 1424293 | G | A | [symbol] | CDZ98791 |
| X₄₂₈₃ | M4 | 262 | 1455677 | G | A | [symbol] | CDZ98804 |
| X₄₂₈₄ | M4 | 262 | 1458863 | C | T | [symbol] | CDZ98807 |
| X₄₂₈₅ | M4 | 262 | 1550311 | C | T | [symbol] | CDZ98836 |
| X₄₂₈₆ | M4 | 262 | 1550456 | C | T | [symbol] | CDZ98837 |
| X₄₂₈₇ | M4 | 262 | 1577316 | G | A | [symbol] | CDZ98844 |
| X₄₂₈₈ | M4 | 262 | 1615762 | G | A | [symbol] | CDZ98855 |
| X₄₂₈₉ | M4 | 262 | 1677620 | G | A | [symbol] | CDZ98873 |
| X₄₂₉₀ | M4 | 262 | 1694625 | C | T | [symbol] | CDZ98878 |
| X₄₂₉₁ | M4 | 262 | 1711527 | G | A | [symbol] | CDZ98886 |
| X₄₂₉₂ | M4 | 262 | 1716385 | G | A | [symbol] | CDZ98887 |
| X₄₂₉₃ | M4 | 266 | 85 | C | T | [symbol] | |
| X₄₂₉₄ | M4 | 266 | 252 | C | T | [symbol] | |
| X₄₂₉₅ | M4 | 267 | 133 | T | C | [symbol] | |
| X₄₂₉₆ | M4 | 267 | 134 | c | T | [symbol] | |
| X₄₂₉₇ | M4 | 267 | 254 | G | T | [symbol] | |
| X₄₂₉₈ | M4 | 267 | 256 | A | G | [symbol] | |
| X₄₂₉₉ | M4 | 267 | 257 | T | C | [symbol] | |
| X₄₃₀₀ | M5 | 22 | 1643 | C | T | [symbol] | CED80056 |
| X₄₃₀₁ | M5 | 22 | 2524 | G | A | [symbol] | CED80056 |
| X₄₃₀₂ | M5 | 28 | 236 | G | A | [symbol] | CDZ96150 |
| X₄₃₀₃ | M5 | 28 | 377 | G | A | [symbol] | CDZ96150 |
| X₄₃₀₄ | M5 | 28 | 1506 | C | T | [symbol] | CDZ96150 |
| X₄₃₀₅ | M5 | 28 | 1677 | C | T | [symbol] | CDZ96150 |
| X₄₃₀₆ | M5 | 43 | 464 | G | A | [symbol] | CDZ96151 |
| X₄₃₀₇ | M5 | 43 | 753 | C | T | [symbol] | CDZ96151 |
| X₄₃₀₈ | M5 | 43 | 757 | G | A | [symbol] | CDZ96151 |
| X₄₃₀₉ | M5 | 43 | 1527 | C | T | [symbol] | CDZ96151 |
| X₄₃₁₀ | M5 | 43 | 1748 | G | A | [symbol] | CDZ96151 |
| X₄₃₁₁ | M5 | 43 | 1928 | C | T | [symbol] | CDZ96151 |
| X₄₃₁₂ | M5 | 62 | 26 | T | A | [symbol] | CDZ96152 |
| X₄₃₁₃ | M5 | 62 | 27 | T | C | [symbol] | CDZ96152 |
| X₄₃₁₄ | M5 | 62 | 55 | T | A | [symbol] | CDZ96152 |
| X₄₃₁₅ | M5 | 62 | 403 | C | T | [symbol] | CDZ96152 |
| X₄₃₁₆ | M5 | 62 | 875 | G | A | [symbol] | CDZ96152 |
| X₄₃₁₇ | M5 | 102 | 339 | C | T | [symbol] | CDZ96153 |
| X₄₃₁₈ | M5 | 102 | 951 | G | A | [symbol] | CDZ96153 |
| X₄₃₁₉ | M5 | 102 | 1042 | G | A | [symbol] | CDZ96153 |
| X₄₃₂₀ | M5 | 102 | 2334 | G | A | [symbol] | CDZ96153 |
| X₄₃₂₁ | M5 | 102 | 2590 | C | T | [symbol] | CDZ96153 |
| X₄₃₂₂ | M5 | 102 | 3427 | C | T | [symbol] | CDZ96153 |
| X₄₃₂₃ | M5 | 176 | 358 | G | A | [symbol] | CED80058 |
| X₄₃₂₄ | M5 | 176 | 1872 | G | A | [symbol] | CED80058 |
| X₄₃₂₅ | M5 | 176 | 1980 | G | A | [symbol] | CED80058 |
| X₄₃₂₆ | M5 | 179 | 1525 | G | A | [symbol] | CDZ96154 |
| X₄₃₂₇ | M5 | 179 | 1780 | C | T | [symbol] | CDZ96154 |
| X₄₃₂₈ | M5 | 179 | 2472 | G | A | [symbol] | CDZ96154 |
| X₄₃₂₉ | M5 | 179 | 2476 | C | T | [symbol] | CDZ96154 |
| X₄₃₃₀ | M5 | 188 | 815 | C | T | [symbol] | CED80060 |
| X₄₃₃₁ | M5 | 188 | 1174 | C | T | [symbol] | CED80060 |
| X₄₃₃₂ | M5 | 188 | 2163 | G | A | [symbol] | CED80060 |
| X₄₃₃₃ | M5 | 188 | 2170 | G | A | [symbol] | CED80060 |
| X₄₃₃₄ | M5 | 247 | 102 | G | A | [symbol] | CED80061 |
| X₄₃₃₅ | M5 | 247 | 989 | C | T | [symbol] | CED80061 |
| X₄₃₃₆ | M5 | 247 | 1053 | G | A | [symbol] | CED80061 |
| X₄₃₃₇ | M5 | 7 | 160 | T | C | [symbol] | |
| X₄₃₃₈ | M5 | 8 | 1110 | C | T | [symbol] | |
| X₄₃₃₉ | M5 | 11 | 154 | A | C | [symbol] | |
| X₄₃₄₀ | M5 | 11 | 158 | C | T | [symbol] | |
| X₄₃₄₁ | M5 | 15 | 28 | C | G | [symbol] | |
| X₄₃₄₂ | M5 | 24 | 513 | T | C | [symbol] | CDZ96160 |
| X₄₃₄₃ | M3 | 24 | 520 | C | G | [symbol] | CDZ96160 |
| X₄₃₄₄ | M5 | 24 | 5850 | C | T | [symbol] | CDZ96160 |
| X₄₃₄₅ | M5 | 24 | 29899 | C | T | [symbol] | CDZ96169 |
| X₄₃₄₆ | M5 | 24 | 169574 | C | T | [symbol] | CDZ96213 |
| X₄₃₄₇ | M5 | 24 | 172698 | G | A | [symbol] | CDZ96215 |
| X₄₃₄₈ | M5 | 24 | 180340 | G | A | [symbol] | CDZ96216 |
| X₄₃₄₉ | M5 | 24 | 266697 | G | A | [symbol] | CDZ96242 |
| X₄₃₅₀ | M5 | 24 | 307114 | G | A | [symbol] | CDZ96255 |
| X₄₃₅₁ | M5 | 24 | 334227 | T | A | [symbol] | CDZ96263 |
| X₄₃₅₂ | M5 | 24 | 335171 | C | T | [symbol] | CDZ96265 |
| X₄₃₅₃ | M5 | 24 | 340663 | T | C | [symbol] | CDZ96265 |
| X₄₃₅₄ | M5 | 24 | 400418 | C | T | [symbol] | CDZ96287 |
| X₄₃₅₅ | M5 | 24 | 420108 | G | A | [symbol] | CDZ96291 |
| X₄₃₅₆ | M5 | 27 | 445 | C | T | [symbol] | |
| X₄₃₅₇ | M5 | 27 | 1012 | A | C | [symbol] | |
| X₄₃₅₈ | M5 | 27 | 1013 | T | A | [symbol] | |
| X₄₃₅₉ | M5 | 32 | 158 | C | T | [symbol] | CDZ96332 |
| X₄₃₆₀ | M5 | 32 | 215 | C | T | [symbol] | CDZ96332 |
| X₄₃₆₁ | M5 | 32 | 2438 | C | T | [symbol] | CDZ96332 |
| X₄₃₆₂ | M5 | 32 | 2734 | C | T | [symbol] | CDZ96332 |
| X₄₃₆₃ | M5 | 32 | 2821 | C | T | [symbol] | CDZ96332 |
| X₄₃₆₄ | M5 | 32 | 3067 | C | T | [symbol] | CDZ96332 |
| X₄₃₆₅ | M5 | 32 | 3506 | C | T | [symbol] | CDZ96332 |
| X₄₃₆₆ | M5 | 32 | 3562 | G | A | [symbol] | CDZ96332 |
| X₄₃₆₇ | M5 | 33 | 399 | T | A | [symbol] | CED82002 |
| X₄₃₆₈ | M5 | 33 | 20227 | G | A | [symbol] | CED82006 |
| X₄₃₆₉ | M5 | 33 | 45488 | C | T | [symbol] | CED82012 |
| X₄₃₇₀ | M5 | 33 | 64676 | G | A | [symbol] | CED82019 |
| X₄₃₇₁ | M5 | 33 | 79913 | G | A | [symbol] | CED82024 |
| X₄₃₇₂ | M5 | 33 | 105276 | G | A | [symbol] | CED82036 |
| X₄₃₇₃ | M5 | 33 | 109397 | G | A | [symbol] | CED82038 |
| X₄₃₇₄ | M5 | 33 | 129884 | G | A | [symbol] | CED82045 |
| X₄₃₇₅ | M5 | 33 | 198050 | C | T | [symbol] | CED82067 |
| X₄₃₇₆ | M5 | 33 | 205554 | C | T | [symbol] | CED82069 |
| X₄₃₇₇ | M5 | 33 | 223703 | G | A | [symbol] | CED82075 |
| X₄₃₇₈ | M5 | 33 | 236149 | C | T | [symbol] | CED82078 |
| X₄₃₇₉ | M5 | 33 | 257786 | C | T | [symbol] | CED82084 |
| X₄₃₈₀ | M5 | 33 | 260027 | G | A | [symbol] | CED82084 |
| X₄₃₈₁ | M5 | 33 | 274627 | G | A | [symbol] | CED82091 |
| X₄₃₈₂ | M5 | 33 | 283808 | C | T | [symbol] | CED82093 |
| X₄₃₈₃ | M5 | 33 | 304408 | C | T | [symbol] | CED82099 |
| X₄₃₈₄ | M5 | 33 | 317713 | G | A | [symbol] | CED82104 |
| X₄₃₈₅ | M5 | 33 | 326016 | T | C | [symbol] | CED82108 |
| X₄₃₈₆ | M5 | 33 | 327840 | C | T | [symbol] | CED82108 |
| X₄₃₈₇ | M5 | 33 | 341890 | G | A | [symbol] | CED82113 |
| X₄₃₈₈ | M5 | 33 | 371277 | G | A | [symbol] | CED82121- |
| | | | | | | | CED82122 |
| X₄₃₈₉ | M5 | 33 | 374131 | A | G | [symbol] | CED82121- |
| | | | | | | | CED82122 |
| X₄₃₉₀ | M5 | 33 | 374134 | A | G | [symbol] | CED82121- |
| | | | | | | | CED82122 |
| X₄₃₉₁ | M5 | 33 | 374137 | A | T | [symbol] | CED82121- |
| | | | | | | | CED82122 |
| X₄₃₉₂ | M5 | 33 | 374150 | A | G | [symbol] | CED82121- |
| | | | | | | | CED82122 |
| X₄₃₉₃ | M5 | 33 | 374340 | A | G | [symbol] | CED82121- |
| | | | | | | | CED82122 |
| X₄₃₉₄ | M5 | 33 | 375339 | A | G | [symbol] | CED82121- |
| | | | | | | | CED82122 |
| X₄₃₉₅ | M5 | 33 | 375366 | T | C | [symbol] | CED82121- |
| | | | | | | | CED82122 |
| X₄₃₉₆ | M5 | 33 | 382577 | C | T | [symbol] | CED82122 |
| X₄₃₉₇ | M5 | 33 | 383212 | G | A | [symbol] | CED82122 |
| X₄₃₉₈ | M5 | 33 | 383308 | C | T | [symbol] | CED82122 |
| X₄₃₉₉ | M5 | 33 | 385319 | A | C | [symbol] | CED82122 |
| X₄₄₀₀ | M5 | 33 | 412908 | A | G | [symbol] | CED82131 |
| X₄₄₀₁ | M5 | 33 | 428329 | G | A | [symbol] | CED82136 |
| X₄₄₀₂ | M5 | 33 | 448815 | G | A | [symbol] | CED82144 |
| X₄₄₀₃ | M5 | 33 | 453975 | C | T | [symbol] | CED82147 |
| X₄₄₀₄ | M5 | 33 | 455451 | C | T | [symbol] | CED82146 |
| X₄₄₀₅ | M5 | 33 | 505339 | C | T | [symbol] | CED82165 |
| X₄₄₀₆ | M5 | 33 | 509127 | C | T | [symbol] | CED82166 |
| X₄₄₀₇ | M5 | 33 | 570390 | G | A | [symbol] | CED82188 |
| X₄₄₀₈ | M5 | 33 | 626521 | C | T | [symbol] | CED82203 |
| X₄₄₀₉ | M5 | 33 | 639242 | G | A | [symbol] | CED82208 |
| X₄₄₁₀ | M5 | 33 | 643436 | G | A | [symbol] | CED82208 |
| X₄₄₁₁ | M5 | 33 | 693516 | C | T | [symbol] | CED82225 |
| X₄₄₁₂ | M5 | 33 | 699423 | C | T | [symbol] | CED82227 |
| X₄₄₁₃ | M5 | 33 | 729705 | C | T | [symbol] | CED82234 |
| X₄₄₁₄ | M5 | 33 | 794549 | C | T | [symbol] | CED82254 |
| X₄₄₁₅ | M5 | 33 | 800536 | G | A | [symbol] | CED82259 |
| X₄₄₁₆ | M5 | 33 | 829825 | C | T | [symbol] | CED82271 |
| X₄₄₁₇ | M5 | 33 | 850373 | G | A | [symbol] | CED82278 |
| X₄₄₁₈ | M5 | 33 | 911146 | G | C | [symbol] | CED82293 |
| X₄₄₁₉ | M5 | 33 | 917545 | A | G | [symbol] | CED82294 |
| X₄₄₂₀ | M5 | 33 | 917558 | G | A | [symbol] | CED82294 |
| X₄₄₂₁ | M5 | 33 | 917586 | T | A | [symbol] | CED82294 |
| X₄₄₂₂ | M5 | 33 | 917771 | A | T | [symbol] | CED82294 |
| X₄₄₂₃ | M5 | 33 | 936350 | T | A | [symbol] | CED82300 |
| X₄₄₂₄ | M5 | 33 | 936571 | A | G | [symbol] | CED82300 |
| X₄₄₂₅ | M5 | 33 | 936600 | G | A | [symbol] | CED82300 |
| X₄₄₂₆ | M5 | 33 | 995886 | G | A | [symbol] | CED82318 |
| X₄₄₂₇ | M5 | 33 | 1038506 | C | T | [symbol] | CED82330 |
| X₄₄₂₈ | M5 | 33 | 1049699 | G | A | [symbol] | CED82334 |
| X₄₄₂₉ | M5 | 33 | 1138444 | C | T | [symbol] | CED82363 |
| X₄₄₃₀ | M5 | 33 | 1165914 | G | A | [symbol] | CED82372 |
| X₄₄₃₁ | M5 | 33 | 1169025 | C | T | [symbol] | CED82372 |
| X₄₄₃₂ | M5 | 33 | 1169999 | G | A | [symbol] | CED82372 |
| X₄₄₃₃ | M5 | 33 | 1183030 | T | C | [symbol] | CED82377 |
| X₄₄₃₄ | M5 | 33 | 1190451 | G | A | [symbol] | CED82378 |
| X₄₄₃₅ | M5 | 33 | 1191727 | C | T | [symbol] | CED82379 |
| X₄₄₃₆ | M5 | 33 | 1246468 | C | T | [symbol] | CED82392 |
| X₄₄₃₇ | M5 | 33 | 1248614 | C | T | [symbol] | CED82393 |
| X₄₄₃₈ | M5 | 33 | 1311181 | G | A | [symbol] | CED82413 |
| X₄₄₃₉ | M5 | 33 | 1316789 | C | T | [symbol] | CED82415 |
| X₄₄₄₀ | M5 | 33 | 1324395 | G | A | [symbol] | CED82417 |
| X₄₄₄₁ | M5 | 33 | 1333932 | G | A | [symbol] | CED82421 |
| X₄₄₄₂ | M5 | 33 | 1339376 | C | T | [symbol] | CED82424 |
| X₄₄₄₃ | M5 | 33 | 1396438 | C | T | [symbol] | CED82442 |
| X₄₄₄₄ | M5 | 33 | 1406738 | G | A | [symbol] | CED82444 |
| X₄₄₄₅ | M5 | 33 | 1411059 | C | T | [symbol] | CED82446 |
| X₄₄₄₆ | M5 | 33 | 1499347 | G | A | [symbol] | CED82472 |
| X₄₄₄₇ | M5 | 33 | 1502644 | G | A | [symbol] | CED82474 |
| X₄₄₄₈ | M5 | 33 | 1515148 | G | A | [symbol] | CED82478 |
| X₄₄₄₉ | M5 | 33 | 1515369 | G | A | [symbol] | CED82478 |
| X₄₄₅₀ | M5 | 33 | 1535309 | C | A | [symbol] | CED82484 |
| X₄₄₅₁ | M5 | 33 | 1535314 | T | G | [symbol] | CED82484 |
| X₄₄₅₂ | M5 | 33 | 1535316 | G | C | [symbol] | CED82484 |
| X₄₄₅₃ | M5 | 33 | 1535434 | A | T | [symbol] | CED82483 |
| X₄₄₅₄ | M5 | 33 | 1537493 | C | A | [symbol] | CED82485 |
| X₄₄₅₅ | M5 | 33 | 1537824 | C | T | [symbol] | CED82483 |
| X₄₄₅₆ | M5 | 33 | 1537835 | G | A | [symbol] | CED82483 |
| X₄₄₅₇ | M5 | 33 | 1544767 | A | C | [symbol] | CED82485- |
| | | | | | | | CED82486 |
| X₄₄₅₈ | M5 | 33 | 1544951 | C | G | [symbol] | CED82485- |
| | | | | | | | CED82486 |
| X₄₄₅₉ | M5 | 33 | 1572498 | C | T | [symbol] | CED82491 |
| X₄₄₆₀ | M5 | 33 | 1587443 | C | T | [symbol] | CED82497 |
| X₄₄₆₁ | M5 | 33 | 1596242 | C | T | [symbol] | CED82498 |
| X₄₄₆₂ | M5 | 33 | 1628812 | C | T | [symbol] | CED82510 |
| X₄₄₆₃ | M5 | 33 | 1638726 | G | A | [symbol] | CED82513 |
| X₄₄₆₄ | M5 | 33 | 1642659 | G | A | [symbol] | CED82515 |
| X₄₄₆₅ | M5 | 33 | 1666039 | G | A | [symbol] | CED82521 |
| X₄₄₆₆ | M5 | 33 | 1682149 | C | T | [symbol] | CED82526 |
| X₄₄₆₇ | M5 | 33 | 1689192 | C | T | [symbol] | CED82529 |
| X₄₄₆₈ | M5 | 33 | 1700000 | G | A | [symbol] | CED82534 |
| X₄₄₆₉ | M5 | 33 | 1714108 | G | A | [symbol] | CED82537 |
| X₄₄₇₀ | M5 | 33 | 1723348 | C | T | [symbol] | CED82539 |
| X₄₄₇₁ | M5 | 33 | 1767636 | C | T | [symbol] | CED82552 |
| X₄₄₇₂ | M5 | 33 | 1823362 | C | T | [symbol] | CED82571 |
| X₄₄₇₃ | M5 | 33 | 1848935 | C | T | [symbol] | CED82579 |
| X₄₄₇₄ | M5 | 33 | 1860991 | G | A | [symbol] | CED82583 |
| X₄₄₇₅ | M5 | 33 | 1861675 | C | T | [symbol] | CED82583 |
| X₄₄₇₆ | M5 | 33 | 1881896 | T | C | [symbol] | CED82588 |
| X₄₄₇₇ | M5 | 33 | 1887401 | C | T | [symbol] | CED82591 |
| X₄₄₇₈ | M5 | 33 | 1888706 | C | T | [symbol] | CED82592 |
| X₄₄₇₉ | M5 | 33 | 1938614 | G | A | [symbol] | CED82612 |
| X₄₄₈₀ | M5 | 33 | 1959142 | G | A | [symbol] | CED82619 |
| X₄₄₈₁ | M5 | 33 | 1975079 | G | A | [symbol] | CED82625 |
| X₄₄₈₂ | M5 | 33 | 2010230 | C | T | [symbol] | CED82636 |
| X₄₄₈₃ | M5 | 33 | 2014235 | C | T | [symbol] | CED82637 |
| X₄₄₈₄ | M5 | 33 | 2044014 | G | A | [symbol] | CED82647 |
| X₄₄₈₅ | M5 | 33 | 2046185 | C | T | [symbol] | CED82650 |
| X₄₄₈₆ | M5 | 33 | 2050847 | G | A | [symbol] | CED82651 |
| X₄₄₈₇ | M5 | 36 | 320 | G | A | [symbol] | |
| X₄₄₈₈ | M5 | 36 | 1056 | G | A | [symbol] | |
| X₄₄₈₉ | M5 | 36 | 1295 | C | T | [symbol] | |
| X₄₄₉₀ | M5 | 36 | 1461 | A | T | [symbol] | |
| X₄₄₉₁ | M5 | 42 | 804 | C | T | [symbol] | CDZ96333 |
| X₄₄₉₂ | M5 | 42 | 1559 | G | A | [symbol] | CDZ96333 |
| X₄₄₉₃ | M5 | 45 | 289 | G | A | [symbol] | |
| X₄₄₉₄ | M5 | 48 | 405 | G | A | [symbol] | |
| X₄₄₉₅ | M5 | 48 | 563 | C | T | [symbol] | |
| X₄₄₉₆ | M5 | 48 | 565 | T | C | [symbol] | |
| X₄₄₉₇ | M5 | 48 | 576 | G | A | [symbol] | |
| X₄₄₉₈ | M5 | 48 | 580 | T | A | [symbol] | |
| X₄₄₉₉ | M5 | 48 | 720 | A | G | [symbol] | |
| X₄₅₀₀ | M5 | 50 | 145 | G | A | [symbol] | |
| X₄₅₀₁ | M5 | 52 | 3470 | G | C | [symbol] | CED82664 |
| X₄₅₀₂ | M5 | 52 | 6285 | C | T | [symbol] | CED82665 |
| X₄₅₀₃ | M5 | 52 | 63859 | C | T | [symbol] | CED82686 |
| X₄₅₀₄ | M5 | 52 | 144382 | C | T | [symbol] | CED82709 |
| X₄₅₀₅ | M5 | 52 | 150382 | C | T | [symbol] | CED82712 |
| X₄₅₀₆ | M5 | 52 | 151974 | C | T | [symbol] | CED82712 |
| X₄₅₀₇ | M5 | 52 | 183986 | C | T | [symbol] | CED82722 |
| X₄₅₀₈ | M5 | 52 | 203253 | A | G | [symbol] | CED82730 |
| X₄₅₀₉ | M5 | 52 | 207354 | C | T | [symbol] | CED82730 |
| X₄₅₁₀ | M5 | 52 | 240075 | G | C | [symbol] | CED82734 |
| X₄₅₁₁ | M5 | 52 | 241012 | A | G | [symbol] | CED82734 |
| X₄₅₁₂ | M5 | 52 | 284733 | G | A | [symbol] | CED82747 |
| X₄₅₁₃ | M5 | 52 | 390095 | G | A | [symbol] | CED82784 |
| X₄₅₁₄ | M5 | 52 | 395374 | G | A | [symbol] | CED82786 |
| X₄₅₁₅ | M5 | 52 | 398723 | C | T | [symbol] | CED82786 |
| X₄₅₁₆ | M5 | 52 | 398728 | C | T | [symbol] | CED82786 |
| X₄₅₁₇ | M5 | 52 | 487472 | C | T | [symbol] | CED82814 |
| X₄₅₁₈ | M5 | 52 | 516267 | C | T | [symbol] | CED82823 |
| X₄₅₁₉ | M5 | 52 | 552099 | G | A | [symbol] | CED82837 |
| X₄₅₂₀ | M5 | 52 | 552173 | G | A | [symbol] | CED82837 |
| X₄₅₂₁ | M5 | 52 | 631479 | C | T | [symbol] | CED82860 |
| X₄₅₂₂ | M5 | 52 | 638622 | G | A | [symbol] | CED82862 |
| X₄₅₂₃ | M5 | 52 | 692048 | C | T | [symbol] | CED82884 |
| X₄₅₂₄ | M5 | 52 | 700204 | G | A | [symbol] | CED82888 |
| X₄₅₂₅ | M5 | 52 | 783981 | C | T | [symbol] | CED82919 |
| X₄₅₂₆ | M5 | 52 | 809156 | C | T | [symbol] | CED82928 |
| X₄₅₂₇ | M5 | 52 | 817452 | T | A | [symbol] | CED82928 |
| X₄₅₂₈ | M5 | 52 | 840755 | C | T | [symbol] | CED82935 |
| X₄₅₂₉ | M5 | 52 | 842352 | C | T | [symbol] | CED82934 |
| X₄₅₃₀ | M5 | 52 | 849671 | G | A | [symbol] | CED82940 |
| X₄₅₃₁ | M5 | 52 | 861376 | C | T | [symbol] | CED82946 |
| X₄₅₃₂ | M5 | 52 | 938650 | G | A | [symbol] | CED82973 |
| X₄₅₃₃ | M5 | 52 | 967316 | A | T | [symbol] | CED82984 |
| X₄₅₃₄ | M5 | 52 | 967701 | T | A | [symbol] | CED82983 |
| X₄₅₃₅ | M5 | 52 | 978727 | C | T | [symbol] | CED82988 |
| X₄₅₃₆ | M5 | 52 | 1003899 | G | A | [symbol] | CED83001 |
| X₄₅₃₇ | M5 | 52 | 1021202 | C | T | [symbol] | CED83005 |
| X₄₅₃₈ | M5 | 52 | 1029498 | G | A | [symbol] | CED83007 |
| X₄₅₃₉ | M5 | 52 | 1043011 | C | T | [symbol] | CED83012 |
| X₄₅₄₀ | M5 | 52 | 1067895 | C | T | [symbol] | CED83020 |
| X₄₅₄₁ | M5 | 52 | 1151398 | G | A | [symbol] | CED83046 |
| X₄₅₄₂ | M5 | 52 | 1158103 | C | T | [symbol] | CED83051 |
| X₄₅₄₃ | M5 | 52 | 1214927 | C | T | [symbol] | CED83068 |
| X₄₅₄₄ | M5 | 52 | 1219894 | C | T | [symbol] | CED83071 |
| X₄₅₄₅ | M5 | 52 | 1255291 | G | A | [symbol] | CED83086 |
| X₄₅₄₆ | M5 | 52 | 1350463 | C | T | [symbol] | CED83118 |
| X₄₅₄₇ | M5 | 52 | 1358630 | C | T | [symbol] | CED83119 |
| X₄₅₄₈ | M5 | 52 | 1363357 | G | A | [symbol] | CED83122 |
| X₄₅₄₉ | M5 | 52 | 1363405 | C | T | [symbol] | CED83123 |
| X₄₅₅₀ | M5 | 52 | 1365822 | C | T | [symbol] | CED83124 |
| X₄₅₅₁ | M5 | 52 | 1389320 | G | A | [symbol] | CED83130 |
| X₄₅₅₂ | M5 | 52 | 1399439 | G | A | [symbol] | CED83134 |
| X₄₅₅₃ | M5 | 52 | 1409725 | G | A | [symbol] | CED83139 |
| X₄₅₅₄ | M5 | 52 | 1458571 | C | T | [symbol] | CED83158 |
| X₄₅₅₅ | M5 | 52 | 1465985 | G | A | [symbol] | CED83159 |
| X₄₅₅₆ | M5 | 52 | 1474315 | G | A | [symbol] | CED83164 |
| X₄₅₅₇ | M5 | 52 | 1474412 | G | A | [symbol] | CED83166 |
| X₄₅₅₈ | M5 | 52 | 1526796 | G | A | [symbol] | CED83181 |
| X₄₅₅₉ | M5 | 52 | 1540879 | C | T | [symbol] | CED83186 |
| X₄₅₆₀ | M5 | 52 | 1546812 | G | A | [symbol] | CED83188 |
| X₄₅₆₁ | M5 | 52 | 1570053 | C | T | [symbol] | CED83200 |
| X₄₅₆₂ | M5 | 52 | 1583655 | C | T | [symbol] | CED83206 |
| X₄₅₆₃ | M5 | 52 | 1621166 | C | T | [symbol] | CED83219 |
| X₄₅₆₄ | M5 | 52 | 1665003 | A | G | [symbol] | CED83235 |
| X₄₅₆₅ | M5 | 52 | 1688685 | C | T | [symbol] | CED83243 |
| X₄₅₆₆ | M5 | 52 | 1708900 | A | G | [symbol] | CED83249 |
| X₄₅₆₇ | M5 | 52 | 1716210 | C | T | [symbol] | CED83250 |
| X₄₅₆₈ | M5 | 52 | 1765533 | G | A | [symbol] | CED83268 |
| X₄₅₆₉ | M5 | 52 | 1793370 | G | A | [symbol] | CED83275 |
| X₄₅₇₀ | M5 | 52 | 1796780 | G | A | [symbol] | CED83279 |
| X₄₅₇₁ | M5 | 52 | 1807309 | G | A | [symbol] | CED83283 |
| X₄₅₇₂ | M5 | 52 | 1871310 | C | T | [symbol] | CED83304 |
| X₄₅₇₃ | M5 | 52 | 1874150 | G | T | [symbol] | CED83305 |
| X₄₅₇₄ | M5 | 52 | 1893352 | G | A | [symbol] | CED83313 |
| X₄₅₇₅ | M5 | 52 | 1915927 | G | A | [symbol] | CED83315 |
| X₄₅₇₆ | M5 | 52 | 1938393 | C | T | [symbol] | CED83322 |
| X₄₅₇₇ | M5 | 52 | 1969873 | C | T | [symbol] | CED83333 |
| X₄₅₇₈ | M5 | 52 | 1987096 | G | A | [symbol] | CED83341 |
| X₄₅₇₉ | M5 | 52 | 1995167 | G | A | [symbol] | CED83342 |
| X₄₅₈₀ | M5 | 52 | 2029893 | G | A | [symbol] | CED83351 |
| X₄₅₈₁ | M5 | 52 | 2041781 | C | T | [symbol] | CED83355 |
| X₄₅₈₂ | M5 | 52 | 2048018 | G | A | [symbol] | CED83358 |
| X₄₅₈₃ | M5 | 52 | 2050245 | G | A | [symbol] | CED83359 |
| X₄₅₈₄ | M5 | 52 | 2071127 | C | T | [symbol] | CED83365 |
| X₄₅₈₅ | M5 | 52 | 2081390 | G | A | [symbol] | CED83367 |
| X₄₅₈₆ | M5 | 52 | 2093148 | C | T | [symbol] | CED83368 |
| X₄₅₈₇ | M5 | 52 | 2093251 | C | T | [symbol] | CED83368 |
| X₄₅₈₈ | M5 | 52 | 2102269 | C | T | [symbol] | CED83371 |
| X₄₅₈₉ | M5 | 52 | 2120665 | C | T | [symbol] | CED83376 |
| X₄₅₉₀ | M5 | 52 | 2136885 | A | G | [symbol] | CED83382 |
| X₄₅₉₁ | M5 | 52 | 2162878 | G | A | [symbol] | CED83392 |
| X₄₅₉₂ | M5 | 52 | 2211680 | G | A | [symbol] | CED83407 |
| X₄₅₉₃ | M5 | 52 | 2229000 | G | A | [symbol] | CED83410 |
| X₄₅₉₄ | M5 | 52 | 2248910 | G | A | [symbol] | CED83417 |
| X₄₅₉₅ | M5 | 52 | 2258192 | C | T | [symbol] | CED83422 |
| X₄₅₉₆ | M5 | 52 | 2265219 | C | T | [symbol] | CED83425 |
| X₄₅₉₇ | M5 | 52 | 2266378 | C | T | [symbol] | CED83423 |
| X₄₅₉₈ | M5 | 52 | 2296638 | G | A | [symbol] | CED83432 |
| X₄₅₉₉ | M5 | 52 | 2327266 | C | T | [symbol] | CED83445 |
| X₄₆₀₀ | M5 | 52 | 2364315 | G | A | [symbol] | CED83455 |
| X₄₆₀₁ | M5 | 52 | 2373120 | C | T | [symbol] | CED83456 |
| X₄₆₀₂ | M5 | 52 | 2400778 | C | T | [symbol] | CED83463 |
| X₄₆₀₃ | M5 | 52 | 2415283 | C | T | [symbol] | CED83469 |
| X₄₆₀₄ | M5 | 52 | 2421337 | G | A | [symbol] | CED83471 |
| X₄₆₀₅ | M5 | 52 | 2482102 | G | A | [symbol] | CED83491 |
| X₄₆₀₆ | M5 | 52 | 2543220 | C | T | [symbol] | CED83509 |
| X₄₆₀₇ | M5 | 53 | 26291 | G | A | [symbol] | CDZ96348 |
| X₄₆₀₈ | M5 | 53 | 55702 | C | T | [symbol] | CDZ96359 |
| X₄₆₀₉ | M5 | 54 | 4815 | G | A | [symbol] | CDZ96384 |
| X₄₆₁₀ | M5 | 54 | 4816 | A | T | [symbol] | CDZ96384 |
| X₄₆₁₁ | M5 | 54 | 4817 | G | C | [symbol] | CDZ96384 |
| X₄₆₁₂ | M5 | 54 | 4821 | G | A | [symbol] | CDZ96384 |
| X₄₆₁₃ | M5 | 54 | 16963 | C | T | [symbol] | CDZ96386 |
| X₄₆₁₄ | M5 | 54 | 83852 | G | A | [symbol] | CDZ96409 |
| X₄₆₁₅ | M5 | 54 | 93810 | G | A | [symbol] | CDZ96414 |
| X₄₆₁₆ | M5 | 54 | 106086 | G | A | [symbol] | CDZ96416 |
| X₄₆₁₇ | M5 | 54 | 194230 | G | A | [symbol] | CDZ96446 |
| X₄₆₁₈ | M5 | 54 | 202467 | C | T | [symbol] | CDZ96448 |
| X₄₆₁₉ | M5 | 54 | 206494 | G | A | [symbol] | CDZ96450 |
| X₄₆₂₀ | M5 | 54 | 207737 | C | T | [symbol] | CDZ96450 |
| X₄₆₂₁ | M5 | 54 | 245840 | G | A | [symbol] | CDZ96464 |
| X₄₆₂₂ | M5 | 54 | 274617 | A | T | [symbol] | CDZ96471 |
| X₄₆₂₃ | M5 | 54 | 278479 | C | T | [symbol] | CDZ96474 |
| X₄₆₂₄ | M5 | 54 | 290948 | C | T | [symbol] | CDZ96479 |
| X₄₆₂₅ | M5 | 54 | 317033 | C | A | [symbol] | CDZ96487 |
| X₄₆₂₆ | M5 | 54 | 354688 | C | T | [symbol] | CDZ96493 |
| X₄₆₂₇ | M5 | 54 | 357141 | G | A | [symbol] | CDZ96496 |
| X₄₆₂₈ | M5 | 54 | 370320 | G | A | [symbol] | CDZ96499 |
| X₄₆₂₉ | M5 | 54 | 383172 | C | T | [symbol] | CDZ96506 |
| X₄₆₃₀ | M5 | 54 | 391223 | A | G | [symbol] | CDZ96509 |
| X₄₆₃₁ | M5 | 54 | 402099 | G | A | [symbol] | CDZ96511 |
| X₄₆₃₂ | M5 | 54 | 413765 | G | A | [symbol] | CDZ96516 |
| X₄₆₃₃ | M5 | 54 | 427542 | C | T | [symbol] | CDZ96520 |
| X₄₆₃₄ | M5 | 54 | 431851 | C | T | [symbol] | CDZ96520 |
| X₄₆₃₅ | M5 | 54 | 437080 | G | A | [symbol] | CDZ96522 |
| X₄₆₃₆ | M5 | 54 | 478527 | C | T | [symbol] | CDZ96537 |
| X₄₆₃₇ | M5 | 54 | 555986 | G | A | [symbol] | CDZ96563 |
| X₄₆₃₈ | M5 | 54 | 564688 | C | T | [symbol] | CDZ96566 |
| X₄₆₃₉ | M5 | 54 | 616268 | A | G | [symbol] | CDZ96581 |
| X₄₆₄₀ | M5 | 54 | 619019 | G | A | [symbol] | CDZ96580 |
| X₄₆₄₁ | M5 | 54 | 648472 | G | A | [symbol] | CDZ96590 |
| X₄₆₄₂ | M5 | 54 | 709210 | G | A | [symbol] | CDZ96613 |
| X₄₆₄₃ | M5 | 54 | 733193 | T | C | [symbol] | CDZ96619 |
| X₄₆₄₄ | M5 | 54 | 743396 | G | A | [symbol] | CDZ96621 |
| X₄₆₄₅ | M5 | 58 | 93 | T | C | [symbol] | |
| X₄₆₄₆ | M5 | 64 | 2756 | A | C | [symbol] | CDZ96626 |
| X₄₆₄₇ | M5 | 64 | 2767 | G | A | [symbol] | CDZ96626 |
| X₄₆₄₈ | M5 | 64 | 2770 | C | T | [symbol] | CDZ96626 |
| X₄₆₄₉ | M5 | 64 | 2783 | C | T | [symbol] | CDZ96626 |
| X₄₆₅₀ | M5 | 67 | 111 | G | A | [symbol] | |
| X₄₆₅₁ | M5 | 69 | 20845 | C | T | [symbol] | CED83532 |
| X₄₆₅₂ | M5 | 69 | 29006 | G | A | [symbol] | CED83534 |
| X₄₆₅₃ | M5 | 69 | 48869 | G | A | [symbol] | CED83542 |
| X₄₆₅₄ | M5 | 69 | 52428 | G | A | [symbol] | CED83541 |
| X₄₆₅₅ | M5 | 69 | 82710 | G | A | [symbol] | CED83554 |
| X₄₆₅₆ | M5 | 69 | 84820 | G | A | [symbol] | CED83555 |
| X₄₆₅₇ | M5 | 69 | 97985 | G | A | [symbol] | CED83559 |
| X₄₆₅₈ | M5 | 69 | 107920 | C | T | [symbol] | CED83561 |
| X₄₆₅₉ | M5 | 69 | 173697 | G | A | [symbol] | CED83585 |
| X₄₆₆₀ | M5 | 69 | 179873 | C | T | [symbol] | CED83585 |
| X₄₆₆₁ | M5 | 69 | 201005 | G | A | [symbol] | CED83595 |
| X₄₆₆₂ | M5 | 69 | 201292 | G | A | [symbol] | CED83595 |
| X₄₆₆₃ | M5 | 69 | 293602 | G | A | [symbol] | CED83626 |
| X₄₆₆₄ | M5 | 69 | 305997 | C | T | [symbol] | CED83631 |
| X₄₆₆₅ | M5 | 69 | 384418 | G | A | [symbol] | CED83656 |
| X₄₆₆₆ | M5 | 69 | 415659 | C | T | [symbol] | CED83665 |
| X₄₆₆₇ | M5 | 69 | 461021 | C | T | [symbol] | CED83680 |
| X₄₆₆₈ | M5 | 69 | 461986 | G | A | [symbol] | CED83680 |
| X₄₆₆₉ | M5 | 69 | 467686 | C | T | [symbol] | CED83681 |
| X₄₆₇₀ | M5 | 69 | 566212 | G | A | [symbol] | CED83713 |
| X₄₆₇₁ | M5 | 69 | 585845 | G | A | [symbol] | CED83722 |
| X₄₆₇₂ | M5 | 69 | 599390 | G | A | [symbol] | CED83728 |
| X₄₆₇₃ | M5 | 69 | 631511 | G | A | [symbol] | CED83742 |
| X₄₆₇₄ | M5 | 69 | 637299 | T | C | [symbol] | CED83744 |
| X₄₆₇₅ | M5 | 69 | 675579 | G | A | [symbol] | CED83760 |
| X₄₆₇₆ | M5 | 69 | 676581 | G | A | [symbol] | CED83760 |
| X₄₆₇₇ | M5 | 69 | 676853 | G | A | [symbol] | CED83759 |
| X₄₆₇₈ | M5 | 69 | 678406 | C | T | [symbol] | CED83759 |
| X₄₆₇₉ | M5 | 69 | 682770 | C | T | [symbol] | CED83763 |
| X₄₆₈₀ | M5 | 69 | 703602 | C | T | [symbol] | CED83770 |
| X₄₆₈₁ | M5 | 69 | 741255 | C | T | [symbol] | CED83781 |
| X₄₆₈₂ | M5 | 69 | 781048 | C | T | [symbol] | CED83793 |
| X₄₆₈₃ | M5 | 69 | 865820 | C | T | [symbol] | CED83814 |
| X₄₆₈₄ | M5 | 69 | 869706 | G | A | [symbol] | CED83815 |
| X₄₆₈₅ | M5 | 69 | 872470 | G | A | [symbol] | CED83815 |
| X₄₆₈₆ | M5 | 69 | 892199 | G | A | [symbol] | CED83822 |
| X₄₆₈₇ | M5 | 69 | 894019 | G | A | [symbol] | CED83821 |
| X₄₆₈₈ | M5 | 69 | 894462 | C | T | [symbol] | CED83821 |
| X₄₆₈₉ | M5 | 69 | 898044 | C | T | [symbol] | CED83823 |
| X₄₆₉₀ | M3 | 69 | 949982 | C | T | [symbol] | CED83842 |
| X₄₆₉₁ | M5 | 69 | 971219 | C | T | [symbol] | CED83847 |
| X₄₆₉₂ | M5 | 69 | 988364 | G | A | [symbol] | CED83857 |
| X₄₆₉₃ | M5 | 69 | 1001408 | G | A | [symbol] | CED83859 |
| X₄₅₉₄ | M5 | 69 | 1021196 | G | A | [symbol] | CED83867 |
| X₄₆₉₅ | M5 | 69 | 1084824 | G | A | [symbol] | CED83891 |
| X₄₆₉₆ | M5 | 69 | 1084835 | C | T | [symbol] | CED83891 |
| X₄₆₉₇ | M5 | 69 | 1084848 | A | G | [symbol] | CED83891 |
| X₄₆₉₈ | M5 | 69 | 1084866 | A | G | [symbol] | CED83891 |
| X₄₆₉₉ | M5 | 69 | 1084931 | C | T | [symbol] | CED83891 |
| X₄₇₀₀ | M5 | 69 | 1102164 | C | T | [symbol] | CED83897 |
| X₄₇₀₁ | M5 | 69 | 1105560 | C | T | [symbol] | CED83899 |
| X₄₇₀₂ | M5 | 69 | 1109785 | C | T | [symbol] | CED83899 |
| X₄₇₀₃ | M5 | 69 | 1109813 | A | G | [symbol] | CED83899 |
| X₄₇₀₄ | M5 | 69 | 1109818 | A | G | [symbol] | CED83899 |
| X₄₇₀₅ | M5 | 69 | 1109819 | T | A | [symbol] | CED83899 |
| X₄₇₀₆ | M5 | 69 | 1109823 | T | C | [symbol] | CED83899 |
| X₄₇₀₇ | M5 | 69 | 1109830 | A | G | [symbol] | CED83899 |
| X₄₇₀₈ | M5 | 69 | 1109831 | C | A | [symbol] | CED83899 |
| X₄₇₀₉ | M5 | 69 | 1109835 | A | G | [symbol] | CED83899 |
| X₄₇₁₀ | M5 | 69 | 1153118 | G | A | [symbol] | CED83915 |
| X₄₇₁₁ | M5 | 69 | 1161041 | A | G | [symbol] | CED83918 |
| X₄₇₁₂ | M5 | 69 | 1179032 | G | A | [symbol] | CED83923 |
| X₄₇₁₃ | M5 | 69 | 1215253 | G | A | [symbol] | CED83936 |
| X₄₇₁₄ | M5 | 69 | 1218409 | G | A | [symbol] | CED83936 |
| X₄₇₁₅ | M5 | 69 | 1260087 | G | A | [symbol] | CED83949 |
| X₄₇₁₆ | M5 | 69 | 1342909 | G | A | [symbol] | CED83975 |
| X₄₇₁₇ | M5 | 69 | 1372486 | C | T | [symbol] | CED83987 |
| X₄₇₁₈ | M5 | 69 | 1396045 | G | A | [symbol] | CED83994 |
| X₄₇₁₉ | M5 | 69 | 1400546 | G | A | [symbol] | CED83995 |
| X₄₇₂₀ | M5 | 69 | 1411237 | C | T | [symbol] | CED84001 |
| X₄₇₂₁ | M5 | 69 | 1411421 | C | T | [symbol] | CED83999 |
| X₄₇₂₂ | M5 | 69 | 1492842 | C | T | [symbol] | CED84026 |
| X₄₇₂₃ | M5 | 69 | 1539964 | C | T | [symbol] | CED84041 |
| X₄₇₂₄ | M5 | 69 | 1568637 | G | A | [symbol] | CED84053 |
| X₄₇₂₅ | M5 | 69 | 1590302 | G | A | [symbol] | CED84059 |
| X₄₇₂₆ | M5 | 69 | 1652288 | C | T | [symbol] | CED84077 |
| X₄₇₂₇ | M5 | 69 | 1674261 | C | T | [symbol] | CED84084 |
| X₄₇₂₈ | M5 | 69 | 1687394 | G | A | [symbol] | CED84087 |
| X₄₇₂₉ | M5 | 69 | 1688555 | C | T | [symbol] | CED84088 |
| X₄₇₃₀ | M5 | 69 | 1701017 | G | A | [symbol] | CED84094 |
| X₄₇₃₁ | M5 | 69 | 1714728 | G | A | [symbol] | CED84097 |
| X₄₇₃₂ | M5 | 69 | 1715549 | G | A | [symbol] | CED84101 |
| X₄₇₃₃ | M5 | 69 | 1720116 | G | A | [symbol] | CED84102 |
| X₄₇₃₄ | M5 | 69 | 1720246 | C | T | [symbol] | CED84101 |
| X₄₇₃₅ | M5 | 69 | 1741179 | C | T | [symbol] | CED84109 |
| X₄₇₃₆ | M5 | 69 | 1790823 | C | T | [symbol] | CED84125 |
| X₄₇₃₇ | M5 | 69 | 1792597 | C | T | [symbol] | CED84126 |
| X₄₇₃₈ | M5 | 69 | 1800584 | G | A | [symbol] | CED84129 |
| X₄₇₃₉ | M5 | 69 | 1803813 | G | A | [symbol] | CED84128 |
| X₄₇₄₀ | M5 | 69 | 1854739 | G | A | [symbol] | CED84145 |
| X₄₇₄₁ | M5 | 69 | 1924621 | C | T | [symbol] | CED84172 |
| X₄₇₄₂ | M5 | 69 | 1967529 | C | T | [symbol] | CED84189 |
| X₄₇₄₃ | M5 | 69 | 1968848 | A | G | [symbol] | CED84189 |
| X₄₇₄₄ | M5 | 69 | 1975946 | G | A | [symbol] | CED84193 |
| X₄₇₄₅ | M5 | 69 | 1978129 | C | G | [symbol] | CED84193 |
| X₄₇₄₆ | M5 | 69 | 2001469 | C | T | [symbol] | CED84197 |
| X₄₇₄₇ | M5 | 69 | 2151569 | C | T | [symbol] | CED84251 |
| X₄₇₄₅ | M5 | 69 | 2160207 | C | T | [symbol] | CED84255 |
| X₄₇₄₉ | M5 | 69 | 2226113 | C | T | [symbol] | CED84272 |
| X₄₇₅₀ | M5 | 69 | 2231926 | G | A | [symbol] | CED84274 |
| X₄₇₅₁ | M5 | 69 | 2240834 | C | T | [symbol] | CED84277 |
| X₄₇₅₂ | M5 | 69 | 2255001 | G | A | [symbol] | CED84282 |
| X₄₇₅₃ | M5 | 69 | 2260222 | C | T | [symbol] | CED84282 |
| X₄₇₅₄ | M5 | 69 | 2325544 | C | T | [symbol] | CED84307 |
| X₄₇₅₅ | M5 | 69 | 2438727 | C | T | [symbol] | CED84343 |
| X₄₇₅₆ | M5 | 69 | 2443210 | T | A | [symbol] | CED84345 |
| X₄₇₅₇ | M5 | 69 | 2483433 | G | A | [symbol] | CED84352 |
| X₄₇₅₈ | M5 | 70 | 22 | T | C | [symbol] | |
| X₄₇₅₉ | M5 | 72 | 93 | T | C | [symbol] | |
| X₄₇₆₀ | M5 | 72 | 584 | G | A | [symbol] | |
| X₄₇₆₁ | M5 | 72 | 856 | C | T | [symbol] | |
| X₄₇₆₂ | M5 | 72 | 1163 | C | T | [symbol] | |
| X₄₇₆₃ | M5 | 77 | 34978 | G | T | [symbol] | CDZ96642 |
| X₄₇₆₄ | M5 | 77 | 39842 | G | A | [symbol] | CDZ96646 |
| X₄₇₆₅ | M5 | 77 | 47151 | C | T | [symbol] | CDZ96648 |
| X₄₇₆₆ | M5 | 77 | 65419 | G | A | [symbol] | CDZ96655 |
| X₄₇₆₇ | M5 | 77 | 84022 | G | A | [symbol] | CDZ96659 |
| X₄₇₆₈ | M5 | 78 | 1865 | C | T | [symbol] | CED84359 |
| X₄₇₆₉ | M5 | 78 | 6333 | G | A | [symbol] | CED84360 |
| X₄₇₇₀ | M5 | 78 | 35200 | G | A | [symbol] | CED84371 |
| X₄₇₇₁ | M5 | 78 | 155757 | C | T | [symbol] | CED84404 |
| X₄₇₇₂ | M5 | 78 | 238854 | C | T | [symbol] | CED84430 |
| X₄₇₇₃ | M5 | 78 | 262683 | G | A | [symbol] | CED84438 |
| X₄₇₇₄ | M5 | 78 | 263828 | T | C | [symbol] | CED84438 |
| X₄₇₇₅ | M5 | 78 | 276239 | T | A | [symbol] | CED84440 |
| X₄₇₇₆ | M5 | 78 | 276361 | T | C | [symbol] | CED84440 |
| X₄₇₇₇ | M5 | 78 | 276362 | C | T | [symbol] | CED84440 |
| X₄₇₇₈ | M5 | 78 | 281454 | C | A | [symbol] | CED84441 |
| X₄₇₇₉ | M5 | 78 | 298141 | C | G | [symbol] | CED84443 |
| X₄₇₈₀ | M5 | 78 | 299644 | G | A | [symbol] | CED84443 |
| X₄₇₈₁ | M5 | 78 | 301815 | T | C | [symbol] | CED84443 |
| X₄₇₈₂ | M5 | 78 | 302700 | A | G | [symbol] | CED84443 |
| X₄₇₈₃ | M5 | 78 | 333312 | A | G | [symbol] | CED84452 |
| X₄₇₈₄ | M5 | 78 | 368561 | G | A | [symbol] | CED84462 |
| X₄₇₈₅ | M5 | 78 | 386356 | C | T | [symbol] | CED84472 |
| X₄₇₈₆ | M5 | 78 | 404988 | G | A | [symbol] | CED84475 |
| X₄₇₈₇ | M5 | 78 | 415432 | G | A | [symbol] | CED84479 |
| X₄₇₈₈ | M5 | 78 | 440606 | C | T | [symbol] | CED84487 |
| X₄₇₈₉ | M5 | 78 | 444292 | C | T | [symbol] | CED84490 |
| X₄₇₉₀ | M5 | 78 | 480848 | C | T | [symbol] | CED84500 |
| X₄₇₉₁ | M5 | 78 | 506703 | G | A | [symbol] | CED84508 |
| X₄₇₉₂ | M5 | 78 | 611362 | C | T | [symbol] | CED84546 |
| X₄₇₉₃ | M5 | 78 | 666005 | C | T | [symbol] | CED84562 |
| X₄₇₉₄ | M5 | 78 | 708376 | C | T | [symbol] | CED84580 |
| X₄₇₉₅ | M5 | 78 | 763771 | G | A | [symbol] | CED84600 |
| X₄₇₉₆ | M5 | 78 | 774252 | G | A | [symbol] | CED84602 |
| X₄₇₉₇ | M5 | 78 | 811274 | G | A | [symbol] | CED84614 |
| X₄₇₉₈ | M5 | 78 | 895505 | C | T | [symbol] | CED84642 |
| X₄₇₉₉ | M5 | 78 | 904287 | G | A | [symbol] | CED84646 |
| X₄₈₀₀ | M5 | 78 | 952815 | G | A | [symbol] | CED84662 |
| X₄₈₀₁ | M5 | 78 | 952860 | G | A | [symbol] | CED84662 |
| X₄₈₀₂ | M5 | 78 | 988551 | G | A | [symbol] | CED84675 |
| X₄₈₀₃ | M5 | 78 | 1023381 | G | A | [symbol] | CED84688 |
| X₄₈₀₄ | M5 | 78 | 1108918 | C | T | [symbol] | CED84721 |
| X₄₈₀₅ | M5 | 78 | 1115511 | G | A | [symbol] | CED84723 |
| X₄₈₀₆ | M5 | 78 | 1216573 | C | T | [symbol] | CED84752 |
| X₄₈₀₇ | M5 | 78 | 1247678 | C | T | [symbol] | CED84761 |
| X₄₈₀₈ | M5 | 78 | 1266077 | G | A | [symbol] | CED84770 |
| X₄₈₀₉ | M5 | 78 | 1302286 | C | T | [symbol] | CED84782 |
| X₄₈₁₀ | M5 | 78 | 1316520 | T | C | [symbol] | CED84788 |
| X₄₈₁₁ | M5 | 78 | 1317124 | G | A | [symbol] | CED84787 |
| X₄₈₁₂ | M5 | 78 | 1344640 | C | T | [symbol] | CED84795 |
| X₄₈₁₃ | M5 | 78 | 1360288 | G | A | [symbol] | CED84802 |
| X₄₈₁₄ | M5 | 78 | 1406981 | C | T | [symbol] | CED84821 |
| X₄₈₁₅ | M5 | 78 | 1410781 | G | A | [symbol] | CED84825 |
| X₄₈₁₆ | M5 | 78 | 1426722 | C | T | [symbol] | CED84829 |
| X₄₈₁₇ | M5 | 78 | 1475803 | G | A | [symbol] | CED84845 |
| X₄₈₁₈ | M5 | 78 | 1495995 | C | T | [symbol] | CED84852 |
| X₄₈₁₉ | M5 | 78 | 1498179 | G | A | [symbol] | CED84852 |
| X₄₈₂₀ | M5 | 78 | 1568326 | C | T | [symbol] | CED84868 |
| X₄₈₂₁ | M5 | 78 | 1607916 | C | T | [symbol] | CED84878 |
| X₄₈₂₂ | M5 | 78 | 1642170 | G | A | [symbol] | CED84889 |
| X₄₈₂₃ | M5 | 78 | 1660408 | G | A | [symbol] | CED84897 |
| X₄₈₂₄ | M5 | 78 | 1694684 | C | T | [symbol] | CED84912 |
| X₄₈₂₅ | M5 | 78 | 1742796 | G | A | [symbol] | CED84926 |
| X₄₈₂₆ | M5 | 79 | 65 | G | A | [symbol] | CDZ96674 |
| X₄₈₂₇ | M5 | 79 | 3779 | G | A | [symbol] | CDZ96674 |
| X₄₈₂₈ | M5 | 79 | 41271 | C | T | [symbol] | CDZ96684 |
| X₄₈₂₉ | M5 | 79 | 47085 | G | A | [symbol] | CDZ96684 |
| X₄₈₃₀ | M5 | 79 | 57804 | G | A | [symbol] | CDZ96687 |
| X₄₈₃₁ | M5 | 79 | 71637 | C | T | [symbol] | CDZ96691 |
| X₄₈₃₂ | M5 | 79 | 132683 | C | T | [symbol] | CDZ96713 |
| X₄₈₃₃ | M5 | 79 | 171814 | C | T | [symbol] | CDZ96727 |
| X₄₈₃₄ | M5 | 79 | 241350 | C | T | [symbol] | CDZ96753 |
| X₄₈₃₅ | M5 | 79 | 281783 | C | T | [symbol] | CDZ96768 |
| X₄₈₃₆ | M5 | 79 | 307021 | C | T | [symbol] | CDZ96777 |
| X₄₈₃₇ | M5 | 79 | 324145 | C | T | [symbol] | CDZ96781 |
| X₄₈₃₈ | M5 | 79 | 346823 | G | A | [symbol] | CDZ96789 |
| X₄₈₃₉ | M5 | 79 | 368176 | G | A | [symbol] | CDZ96795 |
| X₄₈₄₀ | M5 | 79 | 369258 | G | A | [symbol] | CDZ96793 |
| X₄₈₄₁ | M5 | 79 | 489676 | G | A | [symbol] | CDZ96839 |
| X₄₈₄₂ | M5 | 79 | 492523 | C | T | [symbol] | CDZ96842 |
| X₄₈₄₃ | M5 | 79 | 575411 | C | T | [symbol] | CDZ96868 |
| X₄₈₄₄ | M5 | 79 | 579674 | G | A | [symbol] | CDZ96871 |
| X₄₈₄₅ | M5 | 79 | 604739 | G | A | [symbol] | CDZ96879 |
| X₄₈₄₆ | M5 | 79 | 611702 | C | T | [symbol] | CDZ96881 |
| X₄₈₄₇ | M5 | 79 | 617057 | C | T | [symbol] | CDZ96882 |
| X₄₈₄₈ | M5 | 79 | 679140 | C | T | [symbol] | CDZ96904 |
| X₄₈₄₉ | M5 | 79 | 691309 | C | T | [symbol] | CDZ96911 |
| X₄₈₅₀ | M5 | 79 | 696174 | G | A | [symbol] | CDZ96911 |
| X₄₈₅₁ | M5 | 79 | 735053 | T | C | [symbol] | CDZ96928 |
| X₄₈₅₂ | M5 | 79 | 747717 | A | G | [symbol] | CDZ96929 |
| X₄₈₅₃ | M5 | 79 | 749595 | A | C | [symbol] | CDZ96930 |
| X₄₈₅₄ | M5 | 79 | 759403 | C | T | [symbol] | CDZ96931 |
| X₄₈₅₅ | M5 | 79 | 759513 | G | A | [symbol] | CDZ96931 |
| X₄₈₅₆ | M5 | 79 | 759589 | G | A | [symbol] | CDZ96932 |
| X₄₈₅₇ | M5 | 79 | 759659 | G | A | [symbol] | CDZ96931 |
| X₄₈₅₈ | M5 | 79 | 760430 | T | C | [symbol] | CDZ96932 |
| X₄₈₅₉ | M5 | 79 | 760541 | A | G | [symbol] | CDZ96932 |
| X₄₈₆₀ | M5 | 79 | 760891 | A | G | [symbol] | CDZ96932 |
| X₄₈₆₁ | M5 | 79 | 760988 | G | T | [symbol] | CDZ96932 |
| X₄₈₆₂ | M5 | 79 | 761056 | T | C | [symbol] | CDZ96932 |
| X₄₈₆₃ | M5 | 79 | 768537 | A | G | [symbol] | CDZ96933 |
| X₄₈₆₄ | M5 | 79 | 771663 | T | G | [symbol] | CDZ96933 |
| X₄₈₆₅ | M5 | 79 | 776112 | A | G | [symbol] | CDZ96934 |
| X₄₈₆₆ | M5 | 79 | 780012 | T | C | [symbol] | CDZ96934 |
| X₄₈₆₇ | M5 | 79 | 786064 | A | G | [symbol] | CDZ96934 |
| X₄₈₆₈ | M5 | 79 | 798799 | C | T | [symbol] | CDZ96939 |
| X₄₈₆₉ | M5 | 79 | 827202 | G | A | [symbol] | CDZ96946 |
| X₄₈₇₀ | M5 | 79 | 907000 | C | T | [symbol] | CDZ96976 |
| X₄₈₇₁ | M5 | 79 | 914302 | C | T | [symbol] | CDZ96977 |
| X₄₈₇₂ | M5 | 79 | 928047 | G | A | [symbol] | CDZ96986 |
| X₄₈₇₃ | M5 | 79 | 933234 | G | A | [symbol] | CDZ96987 |
| X₄₈₇₄ | M5 | 79 | 959497 | C | T | [symbol] | CDZ96997 |
| X₄₈₇₅ | M5 | 79 | 1000786 | A | G | [symbol] | CDZ97007 |
| X₄₈₇₆ | M5 | 79 | 1045859 | C | T | [symbol] | CDZ97023 |
| X₄₈₇₇ | M5 | 79 | 1052968 | C | T | [symbol] | CDZ97024 |
| X₄₈₇₈ | M5 | 79 | 1081745 | C | T | [symbol] | CDZ97037 |
| X₄₈₇₉ | M5 | 79 | 1086267 | G | A | [symbol] | CDZ97038 |
| X₄₈₈₀ | M5 | 79 | 1117040 | C | T | [symbol] | CDZ97047 |
| X₄₈₈₁ | M5 | 79 | 1123288 | C | T | [symbol] | CDZ97049 |
| X₄₈₈₂ | M5 | 79 | 1140763 | G | A | [symbol] | CDZ97053 |
| X₄₈₈₃ | M5 | 79 | 1209208 | G | A | [symbol] | CDZ97077 |
| X₄₈₈₄ | M5 | 79 | 1229129 | G | A | [symbol] | CDZ97086 |
| X₄₈₈₅ | M5 | 79 | 1231740 | C | T | [symbol] | CDZ97087 |
| X₄₈₈₆ | M5 | 79 | 1254757 | G | A | [symbol] | CDZ97095 |
| X₄₈₈₇ | M5 | 79 | 1284476 | C | T | [symbol] | CDZ97106 |
| X₄₈₈₈ | M5 | 79 | 1318034 | C | T | [symbol] | CDZ97117 |
| X₄₈₈₉ | M5 | 79 | 1336766 | C | T | [symbol] | CDZ97121 |
| X₄₈₉₀ | M5 | 79 | 1367232 | C | T | [symbol] | CDZ97129 |
| X₄₈₉₁ | M5 | 79 | 1394034 | G | A | [symbol] | CDZ97137 |
| X₄₈₉₂ | M5 | 79 | 1414969 | G | A | [symbol] | CDZ97147 |
| X₄₈₉₃ | M5 | 79 | 1430954 | G | A | [symbol] | CDZ97152 |
| X₄₈₉₄ | M5 | 79 | 1447480 | G | A | [symbol] | CDZ97156 |
| X₄₈₉₅ | M5 | 79 | 1517815 | C | T | [symbol] | CDZ97180 |
| X₄₈₉₆ | M5 | 79 | 1533781 | C | T | [symbol] | CDZ97185 |
| X₄₈₉₇ | M5 | 79 | 1540205 | C | T | [symbol] | CDZ97186 |
| X₄₈₉₈ | M5 | 79 | 1624415 | G | A | [symbol] | CDZ97217 |
| X₄₈₉₉ | M5 | 79 | 1633005 | A | T | [symbol] | CDZ97217 |
| X₄₉₀₀ | M5 | 79 | 1658469 | C | T | [symbol] | CDZ97227 |
| X₄₉₀₁ | M5 | 79 | 1674448 | G | A | [symbol] | CDZ97228 |
| X₄₉₀₂ | M5 | 79 | 1694516 | C | T | [symbol] | CDZ97233 |
| X₄₉₀₃ | M5 | 79 | 1696776 | G | A | [symbol] | CDZ97234 |
| X₄₉₀₄ | M5 | 79 | 1704321 | G | A | [symbol] | CDZ97235 |
| X₄₉₀₅ | M5 | 79 | 1797660 | C | T | [symbol] | CDZ97265 |
| X₄₉₀₆ | M5 | 79 | 1797676 | G | A | [symbol] | CDZ97265 |
| X₄₉₀₇ | M5 | 79 | 1809211 | C | T | [symbol] | CDZ97268 |
| X₄₉₀₈ | M5 | 79 | 1819746 | C | T | [symbol] | CDZ97273 |
| X₄₉₀₉ | M5 | 79 | 1842448 | G | A | [symbol] | CDZ97282 |
| X₄₉₁₀ | M5 | 79 | 1850295 | G | A | [symbol] | CDZ97285 |
| X₄₉₁₁ | M5 | 79 | 1854359 | G | A | [symbol] | CDZ97285 |
| X₄₉₁₂ | M5 | 79 | 1855037 | A | G | [symbol] | CDZ97285 |
| X₄₉₁₃ | M5 | 79 | 1859220 | A | G | [symbol] | CDZ97291 |
| X₄₉₁₄ | M5 | 79 | 1864111 | C | T | [symbol] | CDZ97291 |
| X₄₉₁₅ | M5 | 79 | 1912360 | G | A | [symbol] | CDZ97307 |
| X₄₉₁₆ | M5 | 79 | 1930688 | G | A | [symbol] | CDZ97315 |
| X₄₉₁₇ | M5 | 79 | 2013361 | C | T | [symbol] | CDZ97344 |
| X₄₉₁₈ | M5 | 79 | 2027861 | C | T | [symbol] | CDZ97350 |
| X₄₉₁₉ | M5 | 79 | 2089423 | C | T | [symbol] | CDZ97372 |
| X₄₉₂₀ | M5 | 79 | 2115870 | G | A | [symbol] | CDZ97380 |
| X₄₉₂₁ | M5 | 79 | 2116476 | C | T | [symbol] | CDZ97380 |
| X₄₉₂₂ | M5 | 79 | 2121041 | G | A | [symbol] | CDZ97380 |
| X₄₉₂₃ | M5 | 79 | 2152573 | C | T | [symbol] | CDZ97392 |
| X₄₉₂₄ | M5 | 79 | 2159433 | G | T | [symbol] | CDZ97394 |
| X₄₉₂₅ | M5 | 79 | 2188410 | G | A | [symbol] | CDZ97405 |
| X₄₉₂₆ | M5 | 79 | 2268540 | C | T | [symbol] | CDZ97435 |
| X₄₉₂₇ | M5 | 79 | 2271463 | G | A | [symbol] | CDZ97437 |
| X₄₉₂₈ | M5 | 79 | 2294761 | G | A | [symbol] | CDZ97444 |
| X₄₉₂₉ | M5 | 79 | 2315869 | A | C | [symbol] | CDZ97452 |
| X₄₉₃₀ | M5 | 79 | 2317043 | G | A | [symbol] | CDZ97452 |
| X₄₉₃₁ | M5 | 79 | 2323713 | C | T | [symbol] | CDZ97455 |
| X₄₉₃₂ | M5 | 79 | 2339881 | C | G | [symbol] | CDZ97460 |
| X₄₉₃₃ | M5 | 79 | 2340326 | G | A | [symbol] | CDZ97462 |
| X₄₉₃₄ | M5 | 79 | 2340636 | T | C | [symbol] | CDZ97462 |
| X₄₉₃₅ | M5 | 85 | 462 | G | A | [symbol] | |
| X₄₉₃₆ | M5 | 85 | 463 | A | G | [symbol] | |
| X₄₉₃₇ | M5 | 85 | 467 | A | T | [symbol] | |
| X₄₉₃₈ | M5 | 85 | 512 | A | G | [symbol] | |
| X₄₉₃₉ | M5 | 85 | 516 | G | A | [symbol] | |
| X₄₉₄₀ | M5 | 85 | 521 | C | T | [symbol] | |
| X₄₉₄₁ | M5 | 85 | 522 | A | G | [symbol] | |
| X₄₉₄₂ | M5 | 86 | 230 | A | G | [symbol] | |
| X₄₉₄₃ | M5 | 88 | 119 | C | T | [symbol] | CDZ97464 |
| X₄₉₄₄ | M5 | 88 | 196 | G | A | [symbol] | CDZ97464 |
| X₄₉₄₅ | M5 | 88 | 898 | C | A | [symbol] | CDZ97464 |
| X₄₉₄₆ | M5 | 93 | 1037 | A | C | [symbol] | |
| X₄₉₄₇ | M5 | 99 | 245 | G | A | [symbol] | |
| X₄₉₄₈ | M5 | 103 | 768 | C | T | [symbol] | |
| X₄₉₄₉ | M5 | 105 | 258 | A | G | [symbol] | CDZ97474 |
| X₄₉₅₀ | M5 | 109 | 396 | T | C | [symbol] | |
| X₄₉₅₁ | M5 | 118 | 396 | C | T | [symbol] | CDZ97478 |
| X₄₉₅₂ | M5 | 118 | 870 | G | A | [symbol] | CDZ97478 |
| X₄₉₅₃ | M5 | 118 | 1491 | G | A | [symbol] | CDZ97478 |
| X₄₉₅₄ | M5 | 124 | 45141 | G | A | [symbol] | CDZ97491 |
| X₄₉₅₅ | M5 | 124 | 72117 | G | A | [symbol] | CDZ97501 |
| X₄₉₅₆ | M5 | 124 | 72760 | G | A | [symbol] | CDZ97501 |
| X₄₉₅₇ | M5 | 124 | 94611 | G | A | [symbol] | CDZ97507 |
| X₄₉₅₈ | M5 | 124 | 111749 | G | A | [symbol] | CDZ97512 |
| X₄₉₅₉ | M5 | 124 | 156052 | G | A | [symbol] | CDZ97524 |
| X₄₉₆₀ | M5 | 124 | 207992 | C | T | [symbol] | CDZ97541 |
| X₄₉₆₁ | M5 | 124 | 222249 | C | T | [symbol] | CDZ97547 |
| X₄₉₆₂ | M5 | 124 | 253872 | C | T | [symbol] | CDZ97553 |
| X₄₉₆₃ | M5 | 125 | 9173 | C | T | [symbol] | CDZ97563 |
| X₄₉₆₄ | M5 | 125 | 35864 | T | C | [symbol] | CDZ97573 |
| X₄₉₆₅ | M5 | 129 | 299 | G | A | [symbol] | CDZ97577 |
| X₄₉₆₆ | M5 | 129 | 433 | G | A | [symbol] | CDZ97577 |
| X₄₉₆₇ | M5 | 129 | 856 | G | A | [symbol] | CDZ97577 |
| X₄₉₆₈ | M5 | 129 | 1013 | C | T | [symbol] | CDZ97577 |
| X₄₉₆₉ | M5 | 130 | 674 | C | T | [symbol] | CDZ97578 |
| X₄₉₇₀ | M5 | 138 | 123 | G | T | [symbol] | |
| X₄₉₇₁ | M5 | 141 | 10162 | G | A | [symbol] | CDZ97582 |
| X₄₉₇₂ | M5 | 141 | 15764 | C | T | [symbol] | CDZ97587 |
| X₄₉₇₃ | M5 | 149 | 2074 | T | G | [symbol] | CDZ97598 |
| X₄₉₇₄ | M5 | 149 | 2453 | C | T | [symbol] | CDZ97598 |
| X₄₉₇₅ | M5 | 159 | 106 | A | C | [symbol] | |
| X₄₉₇₆ | M5 | 162 | 70025 | C | T | [symbol] | CDZ97625 |
| X₄₉₇₇ | M5 | 162 | 70405 | A | G | [symbol] | CDZ97625 |
| X₄₉₇₈ | M5 | 162 | 93443 | G | A | [symbol] | CDZ97631 |
| X₄₉₇₉ | M5 | 162 | 96194 | G | A | [symbol] | CDZ97631 |
| X₄₉₈₀ | M5 | 162 | 117736 | G | A | [symbol] | CDZ97639 |
| X₄₉₈₁ | M5 | 162 | 199495 | G | A | [symbol] | CDZ97664 |
| X₄₉₈₂ | M5 | 162 | 214099 | G | A | [symbol] | CDZ97671 |
| X₄₉₈₃ | M5 | 162 | 218351 | G | A | [symbol] | CDZ97671 |
| X₄₉₈₄ | M5 | 162 | 223407 | C | T | [symbol] | CDZ97674 |
| X₄₉₈₅ | M5 | 162 | 308369 | T | C | [symbol] | CDZ97702 |
| X₄₉₈₆ | M5 | 162 | 311666 | A | G | [symbol] | CDZ97702 |
| X₄₉₈₇ | M5 | 162 | 311707 | C | G | [symbol] | CDZ97702 |
| X₄₉₈₈ | M5 | 162 | 321070 | C | T | [symbol] | CDZ97703 |
| X₄₉₈₉ | M5 | 162 | 321073 | A | G | [symbol] | CDZ97703 |
| X₄₉₉₀ | M5 | 162 | 321460 | C | G | [symbol] | CDZ97703 |
| X₄₉₉₁ | M5 | 162 | 321463 | A | C | [symbol] | CDZ97703 |
| X₄₉₉₂ | M5 | 162 | 322591 | G | A | [symbol] | CDZ97703 |
| X₄₉₉₃ | M5 | 162 | 323448 | T | G | [symbol] | CDZ97703 |
| X₄₉₉₄ | M5 | 162 | 323735 | A | G | [symbol] | CDZ97703 |
| X₄₉₉₅ | M5 | 162 | 324114 | T | C | [symbol] | CDZ97703 |
| X₄₉₉₆ | M5 | 162 | 324139 | C | T | [symbol] | CDZ97703 |
| X₄₉₉₇ | M5 | 162 | 324568 | A | G | [symbol] | CDZ97703 |
| X₄₉₉₈ | M5 | 162 | 360320 | G | A | [symbol] | CDZ97714 |
| X₄₉₉₉ | M5 | 162 | 398165 | C | T | [symbol] | CDZ97730 |
| X₅₀₀₀ | M5 | 162 | 423180 | C | T | [symbol] | CDZ97740 |
| X₅₀₀₁ | M5 | 162 | 424391 | C | T | [symbol] | CDZ97740 |
| X₅₀₀₂ | M5 | 162 | 444123 | T | C | [symbol] | CDZ97748 |
| X₅₀₀₃ | M5 | 162 | 467207 | C | T | [symbol] | CDZ97755 |
| X₅₀₀₄ | M5 | 162 | 480722 | G | A | [symbol] | CDZ97761 |
| X₅₀₀₅ | M5 | 162 | 487433 | G | A | [symbol] | CDZ97763 |
| X₅₀₀₆ | M5 | 162 | 491906 | G | A | [symbol] | CDZ97764 |
| X₅₀₀₇ | M5 | 162 | 492101 | G | A | [symbol] | CDZ97764 |
| X₅₀₀₈ | M5 | 162 | 501886 | C | T | [symbol] | CDZ97769 |
| X₅₀₀₉ | M5 | 162 | 631700 | G | A | [symbol] | CDZ97811 |
| X₅₀₁₀ | M5 | 162 | 632311 | G | A | [symbol] | CDZ97814 |
| X₅₀₁₁ | M5 | 162 | 633321 | G | A | [symbol] | CDZ97814 |
| X₅₀₁₂ | M5 | 162 | 633633 | G | A | [symbol] | CDZ97814 |
| X₅₀₁₃ | M5 | 162 | 640005 | C | T | [symbol] | CDZ97815 |
| X₅₀₁₄ | M5 | 162 | 721840 | G | A | [symbol] | CDZ97838 |
| X₅₀₁₅ | M5 | 162 | 738675 | G | A | [symbol] | CDZ97842 |
| X₅₀₁₆ | M5 | 162 | 738686 | C | T | [symbol] | CDZ97842 |
| X₅₀₁₇ | M5 | 162 | 808830 | G | A | [symbol] | CDZ97869 |
| X₅₀₁₈ | M5 | 162 | 825209 | G | A | [symbol] | CDZ97877 |
| X₅₀₁₉ | M5 | 162 | 871911 | C | T | [symbol] | CDZ97890 |
| X₅₀₂₀ | M5 | 162 | 914850 | C | T | [symbol] | CDZ97904 |
| X₅₀₂₁ | M5 | 162 | 952036 | T | A | [symbol] | CDZ97918 |
| X₅₀₂₂ | M5 | 185 | 21 | C | T | [symbol] | CDZ97957 |
| X₅₀₂₃ | M5 | 189 | 96257 | C | T | [symbol] | CDZ97987 |
| X₅₀₂₄ | M5 | 189 | 126573 | G | A | [symbol] | CDZ97996 |
| X₅₀₂₅ | M5 | 189 | 127356 | G | A | [symbol] | CDZ97996 |
| X₅₀₂₆ | M5 | 189 | 149091 | G | A | [symbol] | CDZ98004 |
| X₅₀₂₇ | M5 | 189 | 194647 | A | G | [symbol] | CDZ98020 |
| X₅₀₂₈ | M5 | 189 | 194665 | A | G | [symbol] | CDZ98020 |
| X₅₀₂₉ | M5 | 189 | 298487 | C | T | [symbol] | CDZ98056 |
| X₅₀₃₀ | M5 | 189 | 323680 | C | T | [symbol] | CDZ98063 |
| X₅₀₃₁ | M5 | 189 | 371247 | G | A | [symbol] | CDZ98078 |
| X₅₀₃₂ | M5 | 189 | 452298 | G | A | [symbol] | CDZ98106 |
| X₅₀₃₃ | M5 | 191 | 342 | A | G | [symbol] | CDZ98107 |
| X₅₀₃₄ | M5 | 191 | 347 | C | T | [symbol] | CDZ98107 |
| X₅₀₃₅ | M5 | 191 | 427 | C | T | [symbol] | CDZ98107 |
| X₅₀₃₆ | M5 | 191 | 451 | C | T | [symbol] | CDZ98107 |
| X₅₀₃₇ | M5 | 191 | 487 | A | G | [symbol] | CDZ98107 |
| X₅₀₃₈ | M5 | 197 | 624 | C | T | [symbol] | CDZ98111 |
| X₅₀₃₉ | M5 | 198 | 202 | C | A | [symbol] | |
| X₅₀₄₀ | M5 | 198 | 203 | G | A | [symbol] | |
| X₅₀₄₁ | M5 | 198 | 238 | C | T | [symbol] | |
| X₅₀₄₂ | M5 | 198 | 265 | C | T | [symbol] | |
| X₅₀₄₃ | M5 | 199 | 688 | C | T | [symbol] | |
| X₅₀₄₄ | M5 | 199 | 911 | C | T | [symbol] | |
| X₅₀₄₅ | M5 | 199 | 971 | C | T | [symbol] | |
| X₅₀₄₆ | M5 | 199 | 1218 | C | T | [symbol] | |
| X₅₀₄₇ | M5 | 202 | 171 | C | T | [symbol] | |
| X₅₀₄₈ | M5 | 205 | 360 | C | T | [symbol] | CDZ98112 |
| X₅₀₄₉ | M5 | 205 | 981 | C | T | [symbol] | CDZ98112 |
| X₅₀₅₀ | M5 | 207 | 406 | G | A | [symbol] | |
| X₅₀₅₁ | M5 | 208 | 117 | G | A | [symbol] | |
| X₅₀₅₂ | M5 | 208 | 476 | G | A | [symbol] | |
| X₅₀₅₃ | M5 | 208 | 616 | G | A | [symbol] | |
| X₅₀₅₄ | M5 | 208 | 935 | G | A | [symbol] | |
| X₅₀₅₅ | M5 | 209 | 312 | C | T | [symbol] | CDZ98113 |
| X₅₀₅₆ | M5 | 214 | 197 | C | G | [symbol] | CDZ98116 |
| X₅₀₅₇ | M5 | 223 | 93 | G | A | [symbol] | CDZ98118 |
| X₅₀₅₈ | M5 | 223 | 102 | A | G | [symbol] | CDZ98118 |
| X₅₀₅₉ | M5 | 223 | 133 | A | G | [symbol] | CDZ98118 |
| X₅₀₆₀ | M5 | 223 | 150 | T | C | [symbol] | CDZ98118 |
| X₅₀₆₁ | M5 | 223 | 155 | A | C | [symbol] | CDZ98118 |
| X₅₀₆₂ | M5 | 223 | 158 | T | C | [symbol] | CDZ98118 |
| X₅₀₆₃ | M5 | 223 | 160 | C | T | [symbol] | CDZ98118 |
| X₅₀₆₄ | M5 | 223 | 175 | G | A | [symbol] | CDZ98118 |
| X₅₀₆₅ | M5 | 223 | 177 | A | G | [symbol] | CDZ98118 |
| X₅₀₆₆ | M5 | 223 | 291 | C | A | [symbol] | CDZ98118 |
| X₅₀₆₇ | M5 | 225 | 317 | C | T | [symbol] | |
| X₅₀₆₈ | M5 | 227 | 202 | G | A | [symbol] | |
| X₅₀₆₉ | M5 | 232 | 340 | G | A | [symbol] | CDZ98119 |
| X₅₀₇₀ | M5 | 232 | 1379 | C | T | [symbol] | CDZ98119 |
| X₅₀₇₁ | M5 | 232 | 1521 | C | T | [symbol] | CDZ98119 |
| X₅₀₇₂ | M5 | 232 | 1662 | G | A | [symbol] | CDZ98119 |
| X₅₀₇₃ | M5 | 232 | 1761 | C | T | [symbol] | CDZ98119 |
| X₅₀₇₄ | M5 | 232 | 2027 | C | T | [symbol] | CDZ98119 |
| X₅₀₇₅ | M5 | 232 | 3060 | C | T | [symbol] | CDZ98120 |
| X₅₀₇₆ | M5 | 232 | 3284 | C | T | [symbol] | CDZ98119 |
| X₅₀₇₇ | M5 | 232 | 4437 | G | A | [symbol] | CDZ98121 |
| X₅₀₇₈ | M5 | 232 | 4561 | C | T | [symbol] | CDZ98120 |
| X₅₀₇₉ | M5 | 242 | 7334 | C | T | [symbol] | CDZ98125 |
| X₅₀₈₀ | M5 | 242 | 41710 | G | A | [symbol] | CDZ98131 |
| X₅₀₈₁ | M5 | 242 | 50262 | G | A | [symbol] | CDZ98135 |
| X₅₀₈₂ | M5 | 242 | 88037 | G | A | [symbol] | CDZ98147 |
| X₅₀₈₃ | M5 | 242 | 107587 | G | A | [symbol] | CDZ98153 |
| X₅₀₈₄ | M5 | 242 | 168689 | G | A | [symbol] | CDZ98172 |
| X₅₀₈₅ | M5 | 242 | 194983 | C | T | [symbol] | CDZ98179 |
| X₅₀₈₆ | M5 | 242 | 213399 | C | T | [symbol] | CDZ98185 |
| X₅₀₈₇ | M5 | 242 | 213986 | G | A | [symbol] | CDZ98185 |
| X₅₀₈₈ | M5 | 242 | 227271 | C | T | [symbol] | CDZ98191 |
| X₅₀₈₉ | M5 | 242 | 233088 | G | A | [symbol] | CDZ98193 |
| X₅₀₉₀ | M5 | 242 | 233141 | G | A | [symbol] | CDZ98193 |
| X₅₀₉₁ | M5 | 242 | 310353 | G | A | [symbol] | CDZ98216 |
| X₅₀₉₂ | M5 | 242 | 363677 | G | A | [symbol] | CDZ98233 |
| X₅₀₉₃ | M5 | 242 | 376713 | G | A | [symbol] | CDZ98238 |
| X₅₀₉₄ | M5 | 242 | 379812 | C | T | [symbol] | CDZ98238 |
| X₅₀₉₅ | M5 | 242 | 404132 | G | A | [symbol] | CDZ98248 |
| X₅₀₉₆ | M5 | 242 | 405825 | G | A | [symbol] | CDZ98250 |
| X₅₀₉₇ | M5 | 242 | 412676 | G | A | [symbol] | CDZ98249 |
| X₅₀₉₈ | M5 | 242 | 432104 | C | T | [symbol] | CDZ98255 |
| X₅₀₉₉ | M5 | 242 | 485936 | G | A | [symbol] | CDZ98270 |
| X₅₁₀₀ | M5 | 242 | 492389 | G | A | [symbol] | CDZ98274 |
| X₅₁₀₁ | M5 | 242 | 514780 | C | T | [symbol] | CDZ98282 |
| X₅₁₀₂ | M5 | 242 | 542663 | G | A | [symbol] | CDZ98290 |
| X₅₁₀₃ | M5 | 242 | 559999 | G | A | [symbol] | CDZ98297 |
| X₅₁₀₄ | M5 | 242 | 602456 | G | A | [symbol] | CDZ98308 |
| X₅₁₀₅ | M5 | 242 | 602531 | G | A | [symbol] | CDZ98308 |
| X₅₁₀₆ | M5 | 242 | 604704 | C | T | [symbol] | CDZ98309 |
| X₅₁₀₇ | M5 | 246 | 329 | C | G | [symbol] | CDZ98328 |
| X₅₁₀₈ | M5 | 246 | 331 | T | C | [symbol] | CDZ98328 |
| X₅₁₀₉ | M5 | 249 | 206 | G | A | [symbol] | CED84929 |
| X₅₁₁₀ | M5 | 249 | 72016 | C | T | [symbol] | CED84950 |
| X₅₁₁₁ | M5 | 249 | 110060 | C | T | [symbol] | CED84968 |
| X₅₁₁₂ | M5 | 249 | 123789 | C | T | [symbol] | CED84972 |
| X₅₁₁₃ | M5 | 249 | 142920 | G | A | [symbol] | CED84978 |
| X₅₁₁₄ | M5 | 249 | 157550 | C | T | [symbol] | CED84983 |
| X₅₁₁₅ | M5 | 249 | 191304 | C | T | [symbol] | CED84988 |
| X₅₁₁₆ | M5 | 249 | 238318 | C | T | [symbol] | CED85003 |
| X₅₁₁₇ | M5 | 249 | 253494 | C | T | [symbol] | CED85008 |
| X₅₁₁₈ | M5 | 249 | 269707 | G | A | [symbol] | CED85013 |
| X₅₁₁₉ | M5 | 249 | 272075 | C | T | [symbol] | CED85013 |
| X₅₁₂₀ | M5 | 249 | 305975 | G | A | [symbol] | CED85023 |
| X₅₁₂₁ | M5 | 249 | 420458 | C | T | [symbol] | CED85069 |
| X₅₁₂₂ | M5 | 249 | 436175 | G | A | [symbol] | CED85073 |
| X₅₁₂₃ | M5 | 249 | 445429 | G | A | [symbol] | CED85075 |
| X₅₁₂₄ | M5 | 249 | 476060 | A | G | [symbol] | CED85088 |
| X₅₁₂₅ | M5 | 249 | 485782 | C | T | [symbol] | CED85089 |
| X₅₁₂₆ | M5 | 249 | 501310 | C | T | [symbol] | CED85093 |
| X₅₁₂₇ | M5 | 249 | 501510 | T | C | [symbol] | CED85093 |
| X₅₁₂₈ | M5 | 249 | 506067 | A | G | [symbol] | CED85094 |
| X₅₁₂₉ | M5 | 249 | 532943 | A | G | [symbol] | CED85099 |
| X₅₁₃₀ | M5 | 249 | 571922 | C | T | [symbol] | CED85115 |
| X₅₁₃₁ | M5 | 249 | 598579 | C | T | [symbol] | CED85125 |
| X₅₁₃₂ | M5 | 249 | 626627 | G | A | [symbol] | CED85132 |
| X₅₁₃₃ | M5 | 249 | 637944 | G | A | [symbol] | CED85135 |
| X₅₁₃₄ | M5 | 249 | 660408 | G | A | [symbol] | CED85144 |
| X₅₁₃₅ | M5 | 249 | 715551 | G | A | [symbol] | CED85163 |
| X₅₁₃₆ | M5 | 249 | 842136 | C | T | [symbol] | CED85198 |
| X₅₁₃₇ | M5 | 249 | 849958 | C | T | [symbol] | CED85201 |
| X₅₁₃₈ | M5 | 249 | 854220 | G | A | [symbol] | CED85201 |
| X₅₁₃₉ | M5 | 249 | 886063 | G | A | [symbol] | CED85208 |
| X₅₁₄₀ | M5 | 249 | 911274 | C | T | [symbol] | CED85218 |
| X₅₁₄₁ | M5 | 249 | 926107 | C | T | [symbol] | CED85222 |
| X₅₁₄₂ | M5 | 249 | 932606 | C | T | [symbol] | CED85223 |
| X₅₁₄₃ | M5 | 249 | 945434 | C | T | [symbol] | CED85227 |
| X₅₁₄₄ | M5 | 249 | 945727 | C | T | [symbol] | CED85227 |
| X₅₁₄₅ | M5 | 249 | 969333 | G | T | [symbol] | CED85238 |
| X₅₁₄₆ | M5 | 249 | 977170 | C | T | [symbol] | CED85242 |
| X₅₁₄₇ | M5 | 249 | 989520 | C | T | [symbol] | CED85243 |
| X₅₁₄₈ | M5 | 249 | 1014098 | C | T | [symbol] | CED85252 |
| X₅₁₄₉ | M5 | 249 | 1014926 | C | T | [symbol] | CED85252 |
| X₅₁₅₀ | M5 | 249 | 1063615 | C | T | [symbol] | CED85265 |
| X₅₁₅₁ | M5 | 249 | 1089152 | G | A | [symbol] | CED85274 |
| X₅₁₅₂ | M5 | 249 | 1144309 | G | A | [symbol] | CED85291 |
| X₅₁₅₃ | M5 | 249 | 1176977 | G | A | [symbol] | CED85303 |
| X₅₁₅₄ | M5 | 249 | 1195367 | G | A | [symbol] | CED85307 |
| X₅₁₅₅ | M5 | 249 | 1203953 | G | A | [symbol] | CED85311 |
| X₅₁₅₆ | M5 | 249 | 1209489 | C | T | [symbol] | CED85313 |
| X₅₁₅₇ | M5 | 249 | 1230068 | C | T | [symbol] | CED85319 |
| X₅₁₅₈ | M5 | 249 | 1234239 | C | T | [symbol] | CED85319 |
| X₅₁₅₉ | M5 | 249 | 1286733 | G | A | [symbol] | CED85336 |
| X₅₁₆₀ | M5 | 249 | 1299472 | C | T | [symbol] | CED85343 |
| X₅₁₆₁ | M5 | 249 | 1310059 | C | T | [symbol] | CED85346 |
| X₅₁₆₂ | M5 | 249 | 1331428 | G | A | [symbol] | CED85352 |
| X₅₁₆₃ | M5 | 249 | 1391343 | G | A | [symbol] | CED85372 |
| X₅₁₆₄ | M5 | 249 | 1393971 | G | A | [symbol] | CED85373 |
| X₅₁₆₅ | M5 | 249 | 1437896 | G | A | [symbol] | CED85389 |
| X₅₁₆₆ | M5 | 249 | 1437902 | G | A | [symbol] | CED85389 |
| X₅₁₆₇ | M5 | 249 | 1457529 | G | A | [symbol] | CED85398 |
| X₅₁₆₈ | M5 | 249 | 1471661 | C | T | [symbol] | CED85402 |
| X₅₁₆₉ | M5 | 249 | 1475590 | G | A | [symbol] | CED85404 |
| X₅₁₇₀ | M5 | 249 | 1498029 | T | C | [symbol] | CED85415 |
| X₅₁₇₁ | M5 | 249 | 1498823 | T | C | [symbol] | CED85414 |
| X₅₁₇₂ | M5 | 249 | 1498829 | C | T | [symbol] | CED85414 |
| X₅₁₇₃ | M5 | 249 | 1499741 | G | A | [symbol] | CED85415 |
| X₅₁₇₄ | M5 | 249 | 1502631 | G | T | [symbol] | CED85416 |
| X₅₁₇₅ | M5 | 249 | 1502641 | T | A | [symbol] | CED85416 |
| X₅₁₇₆ | M5 | 249 | 1502819 | T | G | [symbol] | CED85416 |
| X₅₁₇₇ | M5 | 249 | 1505634 | T | G | [symbol] | CED85416 |
| X₅₁₇₈ | M5 | 24 | 1518591 | T | A | [symbol] | CED85419 |
| X₅₁₇₉ | M5 | 249 | 1519431 | A | G | [symbol] | CED85419 |
| X₅₁₈₀ | M5 | 249 | 1520420 | G | C | [symbol] | CED85420 |
| X₅₁₈₁ | M5 | 249 | 1531574 | C | T | [symbol] | CED85423 |
| X₅₁₈₂ | M5 | 249 | 1599907 | C | T | [symbol] | CED85444 |
| X₅₁₈₃ | M5 | 249 | 1619036 | G | A | [symbol] | CED85450 |
| X₅₁₈₄ | M5 | 249 | 1636931 | G | A | [symbol] | CED85455 |
| X₅₁₈₅ | M5 | 249 | 1653615 | T | G | [symbol] | CED85457 |
| X₅₁₈₆ | M5 | 249 | 1653962 | C | T | [symbol] | CED85458 |
| X₅₁₈₇ | M5 | 249 | 1655479 | C | T | [symbol] | CED85458 |
| X₅₁₈₈ | M5 | 249 | 1666185 | C | T | [symbol] | CED85460 |
| X₅₁₈₉ | M5 | 249 | 1691139 | G | T | [symbol] | CED85467 |
| X₅₁₉₀ | M5 | 249 | 1726777 | C | T | [symbol] | CED85474 |
| X₅₁₉₁ | M5 | 249 | 1766819 | C | T | [symbol] | CED85490 |
| X₅₁₉₂ | M5 | 249 | 1780551 | A | G | [symbol] | CED85493 |
| X₅₁₉₃ | M5 | 249 | 1780569 | A | G | [symbol] | CED85493 |
| X₅₁₉₄ | M5 | 249 | 1801965 | G | A | [symbol] | CED85501 |
| X₅₁₉₅ | M5 | 249 | 1803575 | C | T | [symbol] | CED85502 |
| X₅₁₉₆ | M5 | 249 | 1823713 | A | G | [symbol] | CED85506 |
| X₅₁₉₇ | M5 | 249 | 1862072 | C | T | [symbol] | CED85520 |
| X₅₁₉₈ | M5 | 249 | 1880762 | G | A | [symbol] | CED85525 |
| X₅₁₉₉ | M5 | 249 | 1944026 | G | A | [symbol] | CED85545 |
| X₅₂₀₀ | M5 | 249 | 1951259 | G | A | [symbol] | CED85547 |
| X₅₂₀₁ | M5 | 249 | 1988505 | C | T | [symbol] | CED85560 |
| X₅₂₀₂ | M5 | 249 | 2015272 | G | A | [symbol] | CED85567 |
| X₅₂₀₃ | M5 | 249 | 2021262 | G | A | [symbol] | CED85569 |
| X₅₂₀₄ | M5 | 249 | 2027477 | G | A | [symbol] | CED85573 |
| X₅₂₀₅ | M5 | 249 | 2054064 | C | T | [symbol] | CED85583 |
| X₅₂₀₆ | M5 | 249 | 2110956 | C | T | [symbol] | CED85603 |
| X₅₂₀₇ | M5 | 249 | 2124517 | C | T | [symbol] | CED85607 |
| X₅₂₀₈ | M5 | 249 | 2167664 | G | A | [symbol] | CED85620 |
| X₅₂₀₉ | M5 | 249 | 2178506 | G | A | [symbol] | CED85622 |
| X₅₂₁₀ | M5 | 249 | 2183695 | G | A | [symbol] | CED85623 |
| X₅₂₁₁ | M5 | 249 | 2198205 | G | A | [symbol] | CED85628 |
| X₅₂₁₂ | M5 | 249 | 2223626 | G | A | [symbol] | CED85633 |
| X₅₂₁₃ | M5 | 249 | 2254072 | G | A | [symbol] | CED85640 |
| X₅₂₁₄ | M5 | 249 | 2255547 | G | C | [symbol] | CED85640 |
| X₅₂₁₅ | M5 | 252 | 512 | C | T | [symbol] | |
| X₅₂₁₆ | M5 | 252 | 524 | C | T | [symbol] | |
| X₅₂₁₇ | M5 | 258 | 576 | A | G | [symbol] | |
| X₅₂₁₈ | M5 | 258 | 676 | T | C | [symbol] | |
| X₅₂₁₉ | M5 | 258 | 1139 | A | T | [symbol] | |
| X₅₂₂₀ | M5 | 258 | 1394 | G | A | [symbol] | |
| X₅₂₂₁ | M5 | 260 | 156 | G | A | [symbol] | |
| X₅₂₂₂ | M5 | 262 | 4049 | G | A | [symbol] | CDZ98330 |
| X₅₂₂₃ | M5 | 262 | 30347 | G | A | [symbol] | CDZ98338 |
| X₅₂₂₄ | M5 | 262 | 30359 | G | A | [symbol] | CDZ98338 |
| X₅₂₂₅ | M5 | 262 | 63941 | G | A | [symbol] | CDZ98349 |
| X₅₂₂₆ | M5 | 262 | 134217 | G | A | [symbol] | CDZ98371 |
| X₅₂₂₇ | M5 | 262 | 139442 | C | T | [symbol] | CDZ98375 |
| X₅₂₂₈ | M5 | 262 | 144566 | G | A | [symbol] | CDZ98375 |
| X₅₂₂₉ | M5 | 262 | 148151 | C | T | [symbol] | CDZ98375 |
| X₅₂₃₀ | M5 | 262 | 148199 | G | A | [symbol] | CDZ98376 |
| X₅₂₃₁ | M5 | 262 | 164438 | G | A | [symbol] | CDZ98383 |
| X₅₂₃₂ | M5 | 262 | 201531 | G | A | [symbol] | CDZ98396 |
| X₅₂₃₃ | M5 | 262 | 252708 | G | A | [symbol] | CDZ98414 |
| X₅₂₃₄ | M5 | 262 | 286191 | C | T | [symbol] | CDZ98425 |
| X₅₂₃₅ | M5 | 262 | 319302 | G | A | [symbol] | CDZ98436 |
| X₅₂₃₆ | M5 | 262 | 350852 | C | T | [symbol] | CDZ98449 |
| X₅₂₃₇ | M5 | 262 | 351354 | G | A | [symbol] | CDZ98450 |
| X₅₂₃₈ | M5 | 262 | 366171 | G | A | [symbol] | CDZ98452 |
| X₅₂₃₉ | M5 | 262 | 380519 | C | T | [symbol] | CDZ98457 |
| X₅₂₄₀ | M5 | 262 | 397539 | G | A | [symbol] | CDZ98463 |
| X₅₂₄₁ | M5 | 262 | 414403 | C | T | [symbol] | CDZ98467 |
| X₅₂₄₂ | M5 | 262 | 415802 | C | T | [symbol] | CDZ98467 |
| X₅₂₄₃ | M5 | 262 | 420102 | G | A | [symbol] | CDZ98468 |
| X₅₂₄₄ | M5 | 262 | 446956 | C | T | [symbol] | CDZ98477 |
| X₅₂₄₅ | M5 | 262 | 457461 | T | A | [symbol] | CDZ98483 |
| X₅₂₄₆ | M5 | 262 | 510643 | G | A | [symbol] | CDZ98502 |
| X₅₂₄₇ | M5 | 262 | 539428 | G | A | [symbol] | CDZ98514 |
| X₅₂₄₈ | M5 | 262 | 539452 | A | G | [symbol] | CDZ98514 |
| X₅₂₄₉ | M5 | 262 | 539470 | A | G | [symbol] | CDZ98514 |
| X₅₂₅₀ | M5 | 262 | 552414 | C | T | [symbol] | CDZ98518 |
| X₅₂₅₁ | M5 | 262 | 557280 | G | A | [symbol] | CDZ98518 |
| X₅₂₅₂ | M5 | 262 | 560713 | G | A | [symbol] | CDZ98519 |
| X₅₂₅₃ | M5 | 262 | 562795 | C | T | [symbol] | CDZ98521 |
| X₅₂₅₄ | M5 | 262 | 567591 | G | A | [symbol] | CDZ98524 |
| X₅₂₅₅ | M5 | 262 | 567801 | G | A | [symbol] | CDZ98524 |
| X₅₂₅₆ | M5 | 262 | 607661 | G | A | [symbol] | CDZ98533 |
| X₅₂₅₇ | M5 | 262 | 618198 | G | A | [symbol] | CDZ98538 |
| X₅₂₅₈ | M5 | 262 | 638804 | G | C | [symbol] | CDZ98544 |
| X₅₂₅₉ | M5 | 262 | 639869 | G | C | [symbol] | CDZ98544 |
| X₅₂₆₀ | M5 | 262 | 639962 | A | G | [symbol] | CDZ98544 |
| X₅₂₆₁ | M5 | 262 | 653920 | A | G | [symbol] | CDZ98545 |
| X₅₂₆₂ | M5 | 262 | 655754 | A | T | [symbol] | CDZ98545 |
| X₅₂₆₃ | M5 | 262 | 655757 | T | C | [symbol] | CDZ98545 |
| X₅₂₆₄ | M5 | 262 | 656323 | C | G | [symbol] | CDZ98545 |
| X₅₂₆₅ | M5 | 262 | 668764 | C | T | [symbol] | CDZ98545 |
| X₅₂₆₆ | M5 | 262 | 673556 | G | A | [symbol] | CDZ98548 |
| X₅₂₆₇ | M5 | 262 | 691577 | C | T | [symbol] | CDZ98551 |
| X₅₂₆₈ | M5 | 262 | 733603 | C | T | [symbol] | CDZ98566 |
| X₅₂₆₉ | M5 | 262 | 751209 | C | T | [symbol] | CDZ98572 |
| X₅₂₇₀ | M5 | 262 | 801565 | G | A | [symbol] | CDZ98589 |
| X₅₂₇₁ | M5 | 262 | 822734 | G | A | [symbol] | CDZ98597 |
| X₅₂₇₂ | M5 | 262 | 825723 | A | G | [symbol] | CDZ98598 |
| X₅₂₇₃ | M5 | 262 | 831789 | C | T | [symbol] | CDZ98601 |
| X₅₂₇₄ | M5 | 262 | 835874 | C | T | [symbol] | CDZ98601 |
| X₅₂₇₅ | M5 | 262 | 865999 | G | A | [symbol] | CDZ98613 |
| X₅₂₇₆ | M5 | 262 | 923390 | C | T | [symbol] | CDZ98633 |
| X₅₂₇₇ | M5 | 262 | 971713 | G | A | [symbol] | CDZ98649 |
| X₅₂₇₈ | M5 | 262 | 976074 | C | T | [symbol] | CDZ98650 |
| X₅₂₇₉ | M5 | 262 | 978252 | T | C | [symbol] | CDZ98651 |
| X₅₂₈₀ | M5 | 262 | 1044256 | G | A | [symbol] | CDZ98669 |
| X₅₂₈₁ | M5 | 262 | 1209662 | G | A | [symbol] | CDZ98723 |
| X₅₂₈₂ | M5 | 262 | 1226072 | C | T | [symbol] | CDZ98730 |
| X₅₂₈₃ | M5 | 262 | 1277604 | G | A | [symbol] | CDZ98747 |
| X₅₂₈₄ | M5 | 262 | 1305361 | G | A | [symbol] | CDZ98753 |
| X₅₂₈₅ | M5 | 262 | 1329643 | C | T | [symbol] | CDZ98760 |
| X₅₂₈₆ | M5 | 262 | 1351116 | A | G | [symbol] | CDZ98766 |
| X₅₂₈₇ | M5 | 262 | 1415479 | C | T | [symbol] | CDZ98789 |
| X₅₂₈₈ | M5 | 262 | 1424293 | G | A | [symbol] | CDZ98791 |
| X₅₂₈₉ | M5 | 262 | 1455677 | G | A | [symbol] | CDZ98804 |
| X₅₂₉₀ | M5 | 262 | 1458863 | C | T | [symbol] | CDZ98807 |
| X₅₂₉₁ | M5 | 262 | 1549845 | C | T | [symbol] | CDZ98837 |
| X₅₂₉₂ | M5 | 262 | 1550311 | C | T | [symbol] | CDZ98836 |
| X₅₂₉₃ | M5 | 262 | 1550456 | C | T | [symbol] | CDZ98837 |
| X₅₂₉₄ | M5 | 262 | 1558074 | G | A | [symbol] | CDZ98840 |
| X₅₂₉₅ | M5 | 262 | 1592640 | C | T | [symbol] | CDZ98849 |
| X₅₂₉₆ | M5 | 262 | 1615762 | G | A | [symbol] | CDZ98855 |
| X₅₂₉₇ | M5 | 262 | 1677620 | G | A | [symbol] | CDZ98873 |
| X₅₂₉₈ | M5 | 262 | 1694625 | C | T | [symbol] | CDZ98878 |
| X₅₂₉₉ | M5 | 262 | 1695377 | C | T | [symbol] | CDZ98878 |
| X₅₃₀₀ | M5 | 262 | 1706076 | G | A | [symbol] | CDZ98885 |
| X₅₃₀₁ | M5 | 262 | 1709988 | G | A | [symbol] | CDZ98884 |
| X₅₃₀₂ | M5 | 262 | 1712334 | G | A | [symbol] | CDZ98886 |
| X₅₃₀₃ | M5 | 262 | 1716385 | G | A | [symbol] | CDZ98887 |
| X₅₃₀₄ | M5 | 266 | 252 | C | T | [symbol] | |
| X₅₃₀₅ | M5 | 267 | 93 | G | A | [symbol] | |
| X₅₃₀₆ | M5 | 267 | 94 | A | G | [symbol] | |
| X₅₃₀₇ | M5 | 267 | 133 | T | C | [symbol] | |
| X₅₃₀₈ | M5 | 267 | 134 | C | T | [symbol] | |
| X₅₃₀₉ | M6 | 22 | 1643 | C | T | [symbol] | CED80056 |
| X₅₃₁₀ | M6 | 22 | 2524 | G | A | [symbol] | CED80056 |
| X₅₃₁₁ | M6 | 28 | 236 | G | A | [symbol] | CDZ96150 |
| X₅₃₁₂ | M6 | 28 | 377 | G | A | [symbol] | CDZ96150 |
| X₅₃₁₃ | M6 | 28 | 1506 | C | T | [symbol] | CDZ96150 |
| X₅₃₁₄ | M6 | 28 | 1677 | C | T | [symbol] | CDZ96150 |
| X₅₃₁₅ | M6 | 43 | 464 | G | A | [symbol] | CDZ96151 |
| X₅₃₁₆ | M6 | 43 | 753 | C | T | [symbol] | CDZ96151 |
| X₅₃₁₇ | M6 | 4 | 757 | G | A | [symbol] | CDZ96151 |
| X₅₃₁₈ | M6 | 43 | 1527 | C | T | [symbol] | CDZ96151 |
| X₅₃₁₉ | M6 | 43 | 1748 | G | A | [symbol] | CDZ96151 |
| X₅₃₂₀ | M6 | 43 | 1928 | C | T | [symbol] | CDZ96151 |
| X₅₃₂₁ | M6 | 62 | 26 | T | A | [symbol] | CDZ96152 |
| X₅₃₂₂ | M6 | 62 | 27 | T | C | [symbol] | CDZ96152 |
| X₅₃₂₃ | M6 | 62 | 55 | T | A | [symbol] | CDZ96152 |
| X₅₃₂₄ | M6 | 62 | 403 | C | T | [symbol] | CDZ96152 |
| X₅₃₂₅ | M6 | 62 | 875 | G | A | [symbol] | CDZ96152 |
| X₅₃₂₆ | M6 | 102 | 339 | C | T | [symbol] | CDZ96153 |
| X₅₃₂₇ | M6 | 102 | 951 | G | A | [symbol] | CDZ96153 |
| X₅₃₂₈ | M6 | 102 | 1042 | G | A | [symbol] | CDZ96153 |
| X₅₃₂₉ | M6 | 102 | 2334 | G | A | [symbol] | CDZ96153 |
| X₅₃₃₀ | M6 | 102 | 2590 | C | T | [symbol] | CDZ96153 |
| X₅₃₃₁ | M6 | 102 | 3427 | C | T | [symbol] | CDZ96153 |
| X₅₃₃₂ | M6 | 176 | 358 | G | A | [symbol] | CED80058 |
| X₅₃₃₃ | M6 | 176 | 1872 | G | A | [symbol] | CED80058 |
| X₅₃₃₄ | M6 | 176 | 1980 | G | A | [symbol] | CED80058 |
| X₅₃₃₅ | M6 | 176 | 2335 | A | G | [symbol] | CED80058 |
| X₅₃₃₆ | M6 | 176 | 2349 | G | A | [symbol] | CED80058 |
| X₅₃₃₇ | M6 | 179 | 9 | A | G | [symbol] | CDZ96154 |
| X₅₃₃₈ | M6 | 179 | 96 | G | A | [symbol] | CDZ96154 |
| X₅₃₃₉ | M6 | 179 | 1525 | G | A | [symbol] | CDZ96154 |
| X₅₃₄₀ | M6 | 179 | 1780 | C | T | [symbol] | CDZ96154 |
| X₅₃₄₁ | M6 | 188 | 815 | C | T | [symbol] | CED80060 |
| X₅₃₄₂ | M6 | 188 | 1174 | C | T | [symbol] | CED80060 |
| X₅₃₄₃ | M6 | 188 | 2163 | G | A | [symbol] | CED80060 |
| X₅₃₄₄ | M6 | 188 | 2170 | G | A | [symbol] | CED80060 |
| X₅₃₄₅ | M6 | 247 | 102 | G | A | [symbol] | CED80061 |
| X₅₃₄₆ | M6 | 247 | 989 | C | T | [symbol] | CED80061 |
| X₅₃₄₇ | M6 | 247 | 1053 | G | a | [symbol] | CED80061 |
| X₅₃₄₈ | M6 | 7 | 160 | T | C | [symbol] | |
| X₅₃₄₉ | M6 | 8 | 1110 | C | T | [symbol] | |
| X₅₃₅₀ | M6 | 11 | 154 | A | C | [symbol] | |
| X₅₃₅₁ | M6 | 11 | 158 | C | T | [symbol] | |
| X₅₃₅₂ | M6 | 15 | 28 | C | G | [symbol] | |
| X₅₃₅₃ | M6 | 24 | 5850 | C | T | [symbol] | CDZ96160 |
| X₅₃₅₄ | M6 | 24 | 68660 | C | T | [symbol] | CDZ96181 |
| X₅₃₅₅ | M6 | 24 | 118860 | C | T | [symbol] | CDZ96200 |
| X₅₃₅₆ | M6 | 24 | 180340 | G | A | [symbol] | CDZ96216 |
| X₅₃₅₇ | M6 | 24 | 266697 | G | A | [symbol] | CDZ96242 |
| X₅₃₅₈ | M6 | 24 | 307114 | G | A | [symbol] | CDZ96255 |
| X₅₃₅₉ | M6 | 24 | 320852 | A | T | [symbol] | CDZ96259 |
| X₅₃₆₀ | M6 | 24 | 335171 | C | T | [symbol] | CDZ96265 |
| X₅₃₆₁ | M6 | 24 | 340663 | T | C | [symbol] | CDZ96265 |
| X₅₃₆₂ | M6 | 24 | 349340 | C | T | [symbol] | CDZ96270 |
| X₅₃₆₃ | M6 | 24 | 363770 | C | T | [symbol] | CDZ96275 |
| X₅₃₆₄ | M6 | 24 | 420108 | G | A | [symbol] | CDZ96291 |
| X₅₃₆₅ | M6 | 24 | 470687 | G | A | [symbol] | CDZ96308 |
| X₅₃₆₆ | M6 | 24 | 474698 | G | A | [symbol] | CDZ96308 |
| X₅₃₆₇ | M6 | 26 | 245 | G | A | [symbol] | |
| X₅₃₆₈ | M6 | 27 | 445 | C | T | [symbol] | |
| X₅₃₆₉ | M6 | 27 | 1012 | A | C | [symbol] | |
| X₅₃₇₀ | M6 | 27 | 1013 | T | A | [symbol] | |
| X₅₃₇₁ | M6 | 32 | 158 | C | T | [symbol] | CDZ96332 |
| X₅₃₇₂ | M6 | 32 | 215 | C | T | [symbol] | CDZ96332 |
| X₅₃₇₃ | M6 | 32 | 2438 | C | T | [symbol] | CDZ96332 |
| X₅₃₇₄ | M6 | 32 | 2734 | C | T | [symbol] | CDZ96332 |
| X₅₃₇₅ | M6 | 32 | 2821 | C | T | [symbol] | CDZ96332 |
| X₅₃₇₆ | M6 | 32 | 3067 | C | T | [symbol] | CDZ96332 |
| X₅₃₇₇ | M6 | 32 | 3506 | C | T | [symbol] | CDZ96332 |
| X₅₃₇₈ | M6 | 32 | 3562 | G | A | [symbol] | CDZ96332 |
| X₅₃₇₉ | M6 | 33 | 399 | T | A | [symbol] | CED82002 |
| X₅₃₈₀ | M6 | 33 | 45488 | C | T | [symbol] | CED82012 |
| X₅₃₈₁ | M6 | 33 | 79913 | G | A | [symbol] | CED82024 |
| X₅₃₈₂ | M6 | 33 | 105276 | G | A | [symbol] | CED82036 |
| X₅₃₈₃ | M6 | 33 | 109397 | G | A | [symbol] | CED82038 |
| X₅₃₈₄ | M6 | 33 | 192563 | C | T | [symbol] | CED82066 |
| X₅₃₈₅ | M6 | 33 | 198050 | C | T | [symbol] | CED82067 |
| X₅₃₈₆ | M6 | 33 | 205554 | C | T | [symbol] | CED82069 |
| X₅₃₈₇ | M6 | 33 | 215436 | C | T | [symbol] | CED82071 |
| X₅₃₈₈ | M6 | 33 | 223703 | G | A | [symbol] | CED82075 |
| X₅₃₈₉ | M6 | 33 | 226363 | G | A | [symbol] | CED82075 |
| X₅₃₉₀ | M6 | 33 | 257786 | C | T | [symbol] | CED82084 |
| X₅₃₉₁ | M6 | 33 | 260027 | G | A | [symbol] | CED82084 |
| X₅₃₉₂ | M6 | 33 | 274627 | G | A | [symbol] | CED82091 |
| X₅₃₉₃ | M6 | 33 | 283808 | C | T | [symbol] | CED82093 |
| X₅₃₉₄ | M6 | 33 | 304408 | C | T | [symbol] | CED82099 |
| X₅₃₉₅ | M6 | 33 | 311532 | G | A | [symbol] | CED82101 |
| X₅₃₉₆ | M6 | 33 | 312032 | G | A | [symbol] | CED82101 |
| X₅₃₉₇ | M6 | 33 | 318242 | G | A | [symbol] | CED82105 |
| X₅₃₉₈ | M6 | 33 | 326016 | T | C | [symbol] | CED82108 |
| X₅₃₉₉ | M6 | 33 | 327840 | C | T | [symbol] | CED82108 |
| X₅₄₀₀ | M6 | 33 | 341890 | G | A | [symbol] | CED82113 |
| X₅₄₀₁ | M6 | 33 | 374131 | A | G | [symbol] | CED82121- |
| | | | | | | | CED82122 |
| X₅₄₀₂ | M6 | 33 | 374134 | A | G | [symbol] | CED82121- |
| | | | | | | | CED82122 |
| X₅₄₀₃ | M6 | 33 | 374137 | A | T | [symbol] | CED82121- |
| | | | | | | | CED82122 |
| X₅₄₀₄ | M6 | 33 | 374150 | A | C | [symbol] | CED82121- |
| | | | | | | | CED82122 |
| X₅₄₀₅ | M6 | 33 | 382577 | C | T | [symbol] | CED82122 |
| X₅₄₀₆ | M6 | 33 | 385155 | T | C | [symbol] | CED82122 |
| X₅₄₀₇ | M6 | 33 | 385319 | A | C | [symbol] | CED82122 |
| X₅₄₀₈ | M6 | 33 | 385527 | C | T | [symbol] | CED82122 |
| X₅₄₀₉ | M6 | 33 | 397625 | C | T | [symbol] | CED82125 |
| X₅₄₁₀ | M6 | 33 | 448815 | G | A | [symbol] | CED82144 |
| X₅₄₁₁ | M6 | 33 | 455451 | C | T | [symbol] | CED82146 |
| X₅₄₁₂ | M6 | 33 | 509127 | C | T | [symbol] | CED82166 |
| X₅₄₁₃ | M6 | 33 | 556726 | C | T | [symbol] | CED82183 |
| X₅₄₁₄ | M6 | 33 | 639242 | G | A | [symbol] | CED82208 |
| X₅₄₁₅ | M6 | 33 | 652012 | G | A | [symbol] | CED82212 |
| X₅₄₁₆ | M6 | 33 | 699423 | C | T | [symbol] | CED82227 |
| X₅₄₁₇ | M6 | 33 | 729705 | C | T | [symbol] | CED82234 |
| X₅₄₁₈ | M6 | 33 | 774105 | G | A | [symbol] | CED82248 |
| X₅₄₁₉ | M6 | 33 | 792965 | G | A | [symbol] | CED82256 |
| X₅₄₂₀ | M6 | 33 | 816048 | C | T | [symbol] | CED82262 |
| X₅₄₂₁ | M6 | 33 | 917771 | A | T | [symbol] | CED82294 |
| X₅₄₂₂ | M6 | 33 | 928554 | A | T | [symbol] | CED82297 |
| X₅₄₂₃ | M6 | 33 | 936350 | T | A | [symbol] | CED82300 |
| X₅₄₂₄ | M6 | 33 | 936356 | G | A | [symbol] | CED82300 |
| X₅₄₂₅ | M6 | 33 | 936571 | A | G | [symbol] | CED82300 |
| X₅₄₂₆ | M6 | 33 | 936600 | G | A | [symbol] | CED82300 |
| X₅₄₂₇ | M6 | 33 | 938992 | C | T | [symbol] | CED82300 |
| X₅₄₂₈ | M6 | 33 | 983105 | G | A | [symbol] | CED82315 |
| X₅₄₂₉ | M6 | 33 | 1043825 | C | T | [symbol] | CED82333 |
| X₅₄₃₀ | M6 | 33 | 1049699 | G | A | [symbol] | CED82334 |
| X₅₄₃₁ | M6 | 33 | 1126411 | A | G | [symbol] | CED82358 |
| X₅₄₃₂ | M6 | 33 | 1127676 | G | A | [symbol] | CED82358 |
| X₅₄₃₃ | M6 | 33 | 1138444 | C | T | [symbol] | CED82363 |
| X₅₄₃₄ | M6 | 33 | 1165914 | G | A | [symbol] | CED82372 |
| X₅₄₃₅ | M6 | 33 | 1169999 | G | A | [symbol] | CED82372 |
| X₅₄₃₆ | M6 | 33 | 1191727 | C | T | [symbol] | CED82379 |
| X₅₄₃₇ | M6 | 33 | 1205966 | G | A | [symbol] | CED82382 |
| X₅₄₃₈ | M6 | 33 | 1246468 | C | T | [symbol] | CED82392 |
| X₅₄₃₉ | M6 | 33 | 1277762 | G | A | [symbol] | CED82402 |
| X₅₄₄₀ | M6 | 33 | 1311181 | G | A | [symbol] | CED82413 |
| X₅₄₄₁ | M6 | 33 | 1324395 | G | A | [symbol] | CED82417 |
| X₅₄₄₂ | M6 | 33 | 1333932 | G | A | [symbol] | CED82421 |
| X₅₄₄₃ | M6 | 33 | 1339376 | C | T | [symbol] | CED82424 |
| X₅₄₄₄ | M6 | 33 | 1396438 | C | T | [symbol] | CED82442 |
| X₅₄₄₅ | M6 | 33 | 1399499 | C | T | [symbol] | CED82444 |
| X₅₄₄₆ | M6 | 33 | 1406738 | G | A | [symbol] | CED82444 |
| X₅₄₄₇ | M6 | 33 | 1431034 | G | A | [symbol] | CED82451 |
| X₅₄₄₈ | M6 | 33 | 1470375 | C | T | [symbol] | CED82465 |
| X₅₄₄₉ | M6 | 33 | 1499347 | G | A | [symbol] | CED82472 |
| X₅₄₅₀ | M6 | 33 | 1502644 | G | A | [symbol] | CED82474 |
| X₅₄₅₁ | M6 | 33 | 1544767 | A | C | [symbol] | CED82485- |
| | | | | | | | CED82486 |
| X₅₄₅₂ | M6 | 33 | 1587443 | C | T | [symbol] | CED82497 |
| X₅₄₅₃ | M6 | 33 | 1596242 | C | T | [symbol] | CED82498 |
| X₅₄₅₄ | M6 | 33 | 1638726 | G | A | [symbol] | CED82513 |
| X₅₄₅₅ | M6 | 33 | 1642659 | G | A | [symbol] | CED82515 |
| X₅₄₅₆ | M6 | 33 | 1666106 | C | T | [symbol] | CED82522 |
| X₅₄₅₇ | M6 | 33 | 1682149 | C | T | [symbol] | CED82526 |
| X₅₄₅₈ | M6 | 33 | 1700000 | G | A | [symbol] | CED82534 |
| X₅₄₅₉ | M6 | 33 | 1739035 | G | A | [symbol] | CED82544 |
| X₅₄₆₀ | M6 | 33 | 1767636 | C | T | [symbol] | CED82552 |
| X₅₄₆₁ | M6 | 33 | 1778220 | C | A | [symbol] | CED82556 |
| X₅₄₆₂ | M6 | 33 | 1780044 | G | A | [symbol] | CED82555 |
| X₅₄₆₃ | M6 | 33 | 1823362 | C | T | [symbol] | CED82571 |
| X₅₄₆₄ | M6 | 33 | 1862200 | G | A | [symbol] | CED82582 |
| X₅₄₆₅ | M6 | 33 | 1887401 | C | T | [symbol] | CED82591 |
| X₅₄₆₆ | M6 | 33 | 1888706 | C | T | [symbol] | CED82592 |
| X₅₄₆₇ | M6 | 33 | 1889585 | G | A | [symbol] | CED82592 |
| X₆₄₆₈ | M6 | 33 | 1964763 | G | A | [symbol] | CED82621 |
| X₅₄₆₉ | M6 | 33 | 1975079 | G | A | [symbol] | CED82625 |
| X₅₄₇₀ | M6 | 33 | 2010230 | C | T | [symbol] | CED82636 |
| X₅₄₇₁ | M6 | 33 | 2054662 | G | A | [symbol] | CED82654 |
| X₅₄₇₂ | M6 | 36 | 320 | G | A | [symbol] | |
| X₅₄₇₃ | M6 | 36 | 1056 | G | A | [symbol] | |
| X₅₄₇₄ | M6 | 36 | 1295 | C | T | [symbol] | |
| X₅₄₇₅ | M6 | 36 | 1461 | A | T | [symbol] | |
| X₅₄₇₆ | M6 | 42 | 804 | C | T | [symbol] | CDZ96333 |
| X₅₄₇₇ | M6 | 42 | 1559 | G | A | [symbol] | CDZ96333 |
| X₅₄₇₈ | M6 | 48 | 563 | C | T | [symbol] | |
| X₅₄₇₉ | M6 | 48 | 565 | T | C | [symbol] | |
| X₅₄₈₀ | M6 | 48 | 576 | G | A | [symbol] | |
| X₅₄₈₁ | M6 | 48 | 580 | T | A | [symbol] | |
| X₅₄₈₂ | M6 | 50 | 145 | G | A | [symbol] | |
| X₅₄₈₃ | M6 | 52 | 17234 | A | T | [symbol] | CED82668 |
| X₅₄₈₄ | M6 | 52 | 128353 | G | A | [symbol] | CED82702 |
| X₅₄₈₅ | M6 | 52 | 144382 | C | T | [symbol] | CED82709 |
| X₅₄₈₆ | M6 | 52 | 145060 | G | A | [symbol] | CED82708 |
| X₅₄₈₇ | M6 | 52 | 150382 | C | T | [symbol] | CED82712 |
| X₅₄₈₈ | M6 | 52 | 151974 | C | T | [symbol] | CED82712 |
| X₅₄₈₉ | M6 | 52 | 183986 | C | T | [symbol] | CED82722 |
| X₅₄₉₀ | M6 | 52 | 203253 | A | G | [symbol] | CED82730 |
| X₅₄₉₁ | M6 | 52 | 207354 | C | T | [symbol] | CED82730 |
| X₅₄₉₂ | M6 | 52 | 244305 | G | A | [symbol] | CED82734 |
| X₅₄₉₃ | M6 | 52 | 284733 | G | A | [symbol] | CED82747 |
| X₅₄₉₄ | M6 | 52 | 316848 | C | T | [symbol] | CED82758 |
| X₅₄₉₅ | M6 | 52 | 321045 | C | T | [symbol] | CED82760 |
| X₅₄₉₆ | M6 | 52 | 346628 | G | A | [symbol] | CED82769 |
| X₅₄₉₇ | M6 | 52 | 390095 | G | A | [symbol] | CED82784 |
| X₅₄₉₈ | M6 | 52 | 390988 | C | T | [symbol] | CED82785 |
| X₅₄₉₉ | M6 | 52 | 395374 | G | A | [symbol] | CED82786 |
| X₅₅₀₀ | M6 | 52 | 398723 | C | T | [symbol] | CED82786 |
| X₅₅₀₁ | M6 | 52 | 398728 | C | T | [symbol] | CED82786 |
| X₅₅₀₂ | M6 | 52 | 516267 | C | T | [symbol] | CED82823 |
| X₅₅₀₃ | M6 | 52 | 548788 | G | A | [symbol] | CED82835 |
| X₅₅₀₄ | M6 | 52 | 552099 | G | A | [symbol] | CED82837 |
| X₅₅₀₅ | M6 | 52 | 552173 | G | A | [symbol] | CED82837 |
| X₅₅₀₆ | M6 | 52 | 595806 | C | T | [symbol] | CED82851 |
| X₅₅₀₇ | M6 | 52 | 631479 | C | T | [symbol] | CED82860 |
| X₅₅₀₈ | M6 | 52 | 692048 | C | T | [symbol] | CED82884 |
| X₅₅₀₉ | M6 | 52 | 737403 | G | A | [symbol] | CED82903 |
| X₅₅₁₀ | M6 | 52 | 737419 | G | A | [symbol] | CED82903 |
| X₅₅₁₁ | M6 | 52 | 817452 | T | A | [symbol] | CED82928 |
| X₅₅₁₂ | M6 | 52 | 819924 | C | T | [symbol] | CED82929 |
| X₅₅₁₃ | M6 | 52 | 840755 | C | T | [symbol] | CED82935 |
| X₅₅₁₄ | M6 | 52 | 849671 | G | A | [symbol] | CED82940 |
| X₅₅₁₅ | M6 | 52 | 861376 | C | T | [symbol] | CED82946 |
| X₅₅₁₆ | M6 | 52 | 938650 | G | A | [symbol] | CED82973 |
| X₅₅₁₇ | M6 | 52 | 967316 | A | T | [symbol] | CED82984 |
| X₅₅₁₈ | M6 | 52 | 967701 | T | A | [symbol] | CED82983 |
| X₅₅₁₉ | M6 | 52 | 977048 | G | A | [symbol] | CED82990 |
| X₅₅₂₀ | M6 | 52 | 1003899 | G | A | [symbol] | CED83001 |
| X₅₅₂₁ | M6 | 52 | 1010006 | C | T | [symbol] | CED83002 |
| X₅₅₂₂ | M6 | 52 | 1020804 | G | A | [symbol] | CED83005 |
| X₅₅₂₃ | M6 | 52 | 1021202 | C | T | [symbol] | CED83005 |
| X₅₅₂₄ | M6 | 52 | 1029498 | G | A | [symbol] | CED83007 |
| X₅₅₂₅ | M6 | 52 | 1043011 | C | T | [symbol] | CED83012 |
| X₅₅₂₆ | M6 | 52 | 1067895 | C | T | [symbol] | CED83020 |
| X₅₅₂₇ | M6 | 52 | 1158103 | C | T | [symbol] | CED83051 |
| X₅₅₂₈ | M6 | 52 | 1219894 | C | T | [symbol] | CED83071 |
| X₅₅₂₉ | M6 | 52 | 1363357 | G | A | [symbol] | CED83122 |
| X₅₅₃₀ | M6 | 52 | 1363405 | C | T | [symbol] | CED83123 |
| X₅₅₃₁ | M6 | 52 | 1379155 | G | A | [symbol] | CED83128 |
| X₅₅₃₂ | M6 | 52 | 1389320 | G | A | [symbol] | CED83130 |
| X₅₅₃₃ | M6 | 52 | 1392238 | C | T | [symbol] | CED83130 |
| X₅₅₃₄ | M6 | 52 | 1399439 | G | A | [symbol] | CED83134 |
| X₅₅₃₅ | M6 | 52 | 1481345 | G | A | [symbol] | CED83165 |
| X₅₅₃₆ | M6 | 52 | 1526796 | G | A | [symbol] | CED83181 |
| X₅₅₃₇ | M6 | 52 | 1583655 | C | T | [symbol] | CED83206 |
| X₅₅₃₈ | M6 | 52 | 1590982 | C | T | [symbol] | CED83207 |
| X₅₅₃₉ | M6 | 52 | 1665003 | A | G | [symbol] | CED83235 |
| X₅₅₄₀ | M6 | 52 | 1708900 | A | G | [symbol] | CED83249 |
| X₅₅₄₁ | M6 | 52 | 1715136 | C | T | [symbol] | CED83250 |
| X₅₅₄₂ | M6 | 52 | 1716210 | C | T | [symbol] | CED83250 |
| X₅₅₄₃ | M6 | 52 | 1760440 | G | A | [symbol] | CED83266 |
| X₅₅₄₄ | M6 | 52 | 1793370 | G | A | [symbol] | CED83275 |
| X₅₅₄₅ | M6 | 52 | 1796780 | G | A | [symbol] | CED83279 |
| X₅₅₄₆ | M6 | 52 | 1807309 | G | A | [symbol] | CED83283 |
| X₅₅₄₇ | M6 | 52 | 1861665 | C | T | [symbol] | CED83301 |
| X₅₅₄₈ | M6 | 52 | 1861681 | G | A | [symbol] | CED83301 |
| X₅₅₄₉ | M6 | 52 | 1871310 | C | T | [symbol] | CED83304 |
| X₅₅₅₀ | M6 | 52 | 1874150 | G | T | [symbol] | CED83305 |
| X₅₅₅₁ | M6 | 52 | 1893352 | G | A | [symbol] | CED83313 |
| X₅₅₅₂ | M6 | 52 | 1963875 | G | A | [symbol] | CED83335 |
| X₅₅₅₃ | M6 | 52 | 1987096 | G | A | [symbol] | CED83341 |
| X₅₅₅₄ | M6 | 52 | 2041781 | C | T | [symbol] | CED83355 |
| X₅₅₅₅ | M6 | 52 | 2047954 | G | A | [symbol] | CED83358 |
| X₅₅₅₆ | M6 | 52 | 2048018 | G | A | [symbol] | CED83358 |
| X₅₅₅₇ | M6 | 52 | 2050245 | G | A | [symbol] | CED83359 |
| X₅₅₅₈ | M6 | 52 | 2081390 | G | A | [symbol] | CED83367 |
| X₅₅₅₉ | M6 | 52 | 2120665 | C | T | [symbol] | CED83376 |
| X₅₅₆₀ | M6 | 52 | 2136885 | A | G | [symbol] | CED83382 |
| X₅₅₆₁ | M6 | 52 | 2162878 | G | A | [symbol] | CED83392 |
| X₅₅₆₂ | M6 | 52 | 2222126 | C | T | [symbol] | CED83410 |
| X₅₅₆₃ | M6 | 52 | 2264881 | G | A | [symbol] | CED83424 |
| X₅₅₆₄ | M6 | 52 | 2265219 | C | T | [symbol] | CED83425 |
| X₅₅₆₅ | M6 | 52 | 2266378 | C | T | [symbol] | CED83423 |
| X₅₅₆₆ | M6 | 52 | 2296638 | G | A | [symbol] | CED83432 |
| X₅₅₆₇ | M6 | 52 | 2327266 | C | T | [symbol] | CED83445 |
| X₅₅₆₈ | M6 | 52 | 2330881 | G | A | [symbol] | CED83447 |
| X₅₅₆₉ | M6 | 52 | 2355303 | G | A | [symbol] | CED83453 |
| X₅₅₇₀ | M6 | 52 | 2373120 | C | T | [symbol] | CED83456 |
| X₅₅₇₁ | M6 | 52 | 2400778 | C | T | [symbol] | CED83463 |
| X₅₅₇₂ | M6 | 52 | 2415283 | C | T | [symbol] | CED83469 |
| X₅₅₇₃ | M6 | 52 | 2443575 | G | A | [symbol] | CED83476 |
| X₅₅₇₄ | M6 | 52 | 2575090 | A | G | [symbol] | CED83525 |
| X₅₅₇₅ | M6 | 53 | 26291 | G | A | [symbol] | CDZ96348 |
| X₅₅₇₆ | M6 | 53 | 55702 | C | T | [symbol] | CDZ96359 |
| X₅₅₇₇ | M6 | 54 | 4815 | G | A | [symbol] | CDZ96384 |
| X₅₅₇₈ | M6 | 54 | 4816 | A | T | [symbol] | CDZ96384 |
| X₅₅₇₉ | M6 | 54 | 4817 | G | C | [symbol] | CDZ96384 |
| X₅₅₈₀ | M6 | 54 | 4821 | G | A | [symbol] | CDZ96384 |
| X₅₅₈₁ | M6 | 54 | 60769 | G | A | [symbol] | CDZ96403 |
| X₅₅₈₂ | M6 | 54 | 87947 | G | A | [symbol] | CDZ96410 |
| X₅₅₈₃ | M6 | 54 | 106086 | G | T | [symbol] | CDZ96416 |
| X₅₅₈₄ | M6 | 54 | 114442 | G | A | [symbol] | CDZ96419 |
| X₅₅₈₅ | M6 | 54 | 119439 | C | T | [symbol] | CDZ96420 |
| X₅₅₈₆ | M6 | 54 | 194230 | G | A | [symbol] | CDZ96446 |
| X₅₅₈₇ | M6 | 54 | 195804 | G | A | [symbol] | CDZ96447 |
| X₅₅₈₈ | M6 | 54 | 202467 | C | T | [symbol] | CDZ96448 |
| X₅₅₈₉ | M6 | 54 | 206494 | G | A | [symbol] | CDZ96450 |
| X₅₅₉₀ | M6 | 54 | 207267 | G | A | [symbol] | CDZ96450 |
| X₅₅₉₁ | M6 | 54 | 233698 | G | A | [symbol] | CDZ96457 |
| X₅₅₉₂ | M6 | 54 | 240326 | G | A | [symbol] | CDZ96462 |
| X₅₅₉₃ | M6 | 54 | 258379 | G | A | [symbol] | CDZ96465 |
| X₅₅₉₄ | M6 | 54 | 274617 | A | T | [symbol] | CDZ96471 |
| X₅₅₉₅ | M6 | 54 | 316790 | T | G | [symbol] | CDZ96487 |
| X₅₅₉₆ | M6 | 54 | 357149 | G | A | [symbol] | CDZ96496 |
| X₅₅₉₇ | M6 | 54 | 370320 | G | A | [symbol] | CDZ96499 |
| X₅₅₉₈ | M6 | 54 | 391223 | A | G | [symbol] | CDZ96509 |
| X₅₅₉₉ | M6 | 54 | 402099 | G | A | [symbol] | CDZ96511 |
| X₅₆₀₀ | M6 | 54 | 427542 | C | T | [symbol] | CDZ96520 |
| X₅₆₀₁ | M6 | 54 | 478527 | C | T | [symbol] | CDZ96537 |
| X₅₆₀₂ | M6 | 54 | 564688 | C | T | [symbol] | CDZ96566 |
| X₅₆₀₃ | M6 | 54 | 733193 | T | C | [symbol] | CDZ96619 |
| X₅₆₀₄ | M6 | 54 | 743396 | G | A | [symbol] | CDZ96621 |
| X₅₆₀₅ | M6 | 58 | 93 | T | C | [symbol] | |
| X₅₆₀₆ | M6 | 64 | 2756 | A | C | [symbol] | CDZ96626 |
| X₅₆₀₇ | M6 | 64 | 2767 | G | A | [symbol] | CDZ96626 |
| X₅₆₀₈ | M6 | 64 | 2770 | C | T | [symbol] | CDZ96626 |
| X₅₆₀₉ | M6 | 64 | 2783 | C | T | [symbol] | CDZ96626 |
| X₅₆₁₀ | M6 | 64 | 5037 | G | A | [symbol] | CDZ96626 |
| X₅₆₁₁ | M6 | 67 | 111 | G | A | [symbol] | |
| X₅₆₁₂ | M6 | 67 | 176 | C | T | [symbol] | |
| X₅₆₁₃ | M6 | 69 | 29006 | G | A | [symbol] | CED83534 |
| X₅₆₁₄ | M6 | 69 | 70304 | C | T | [symbol] | CED83551 |
| X₅₆₁₅ | M6 | 69 | 84820 | G | A | [symbol] | CED83555 |
| X₅₆₁₆ | M6 | 69 | 89393 | G | A | [symbol] | CED83556 |
| X₅₆₁₇ | M6 | 69 | 97985 | G | A | [symbol] | CED83559 |
| X₅₆₁₈ | M6 | 69 | 107920 | C | T | [symbol] | CED83561 |
| X₅₆₁₉ | M6 | 69 | 123084 | C | T | [symbol] | CED83563 |
| X₅₆₂₀ | M6 | 69 | 201005 | G | A | [symbol] | CED83595 |
| X₅₆₂₁ | M6 | 69 | 205541 | G | A | [symbol] | CED83597 |
| X₅₆₂₂ | M6 | 69 | 258091 | G | A | [symbol] | CED83614 |
| X₅₆₂₃ | M6 | 69 | 293602 | G | A | [symbol] | CED83626 |
| X₅₆₂₄ | M6 | 69 | 384418 | G | A | [symbol] | CED83656 |
| X₅₆₂₅ | M6 | 69 | 415659 | C | T | [symbol] | CED83665 |
| X₅₆₂₆ | M6 | 69 | 431248 | G | A | [symbol] | CED83671 |
| X₅₆₂₇ | M6 | 69 | 461986 | G | A | [symbol] | CED83680 |
| X₅₆₂₈ | M6 | 69 | 473113 | G | A | [symbol] | CED83684 |
| X₅₆₂₉ | M6 | 69 | 482073 | G | T | [symbol] | CED83685 |
| X₅₆₃₀ | M6 | 69 | 483131 | G | A | [symbol] | CED83687 |
| X₅₆₃₁ | M6 | 69 | 555744 | G | A | [symbol] | CED83711 |
| X₅₆₃₂ | M6 | 69 | 556138 | C | T | [symbol] | CED83710 |
| X₅₆₃₃ | M6 | 69 | 585845 | G | A | [symbol] | CED83722 |
| X₅₆₃₄ | M6 | 69 | 614468 | T | C | [symbol] | CED83733 |
| X₅₆₃₅ | M6 | 69 | 631511 | G | A | [symbol] | CED83742 |
| X₅₆₃₆ | M6 | 69 | 637299 | T | C | [symbol] | CED83744 |
| X₅₆₃₇ | M6 | 69 | 675579 | G | A | [symbol] | CED83760 |
| X₅₆₃₈ | M6 | 69 | 676581 | G | A | [symbol] | CED83760 |
| X₅₆₃₉ | M6 | 69 | 676853 | G | A | [symbol] | CED83759 |
| X₅₆₄₀ | M6 | 69 | 678406 | C | T | [symbol] | CED83759 |
| X₅₆₄₁ | M6 | 69 | 681141 | G | A | [symbol] | CED83762 |
| X₅₆₄₂ | M6 | 69 | 682770 | C | T | [symbol] | CED83763 |
| X₅₆₄₃ | M6 | 69 | 743019 | C | T | [symbol] | CED83782 |
| X₅₆₄₄ | M6 | 69 | 786726 | C | T | [symbol] | CED83794 |
| X₅₆₄₅ | M6 | 69 | 840268 | C | T | [symbol] | CED83808 |
| X₅₆₄₆ | M6 | 69 | 865820 | C | T | [symbol] | CED83814 |
| X₅₆₄₇ | M6 | 69 | 869706 | G | A | [symbol] | CED83815 |
| X₅₆₄₈ | M6 | 69 | 872470 | G | A | [symbol] | CED83815 |
| X₅₆₄₉ | M6 | 69 | 892199 | G | A | [symbol] | CED83822 |
| X₅₆₅₀ | M6 | 69 | 894019 | G | A | [symbol] | CED83821 |
| X₅₆₅₁ | M6 | 69 | 894462 | C | T | [symbol] | CED83821 |
| X₅₆₅₂ | M6 | 69 | 949982 | C | T | [symbol] | CED83842 |
| X₅₆₅₃ | M6 | 69 | 971219 | C | T | [symbol] | CED83847 |
| X₅₆₅₄ | M6 | 69 | 988364 | G | A | [symbol] | CED83857 |
| X₅₆₅₅ | M6 | 69 | 1065858 | G | A | [symbol] | CED83881 |
| X₅₆₅₆ | M6 | 69 | 1084824 | G | A | [symbol] | CED83891 |
| X₅₆₅₇ | M6 | 69 | 1084835 | C | T | [symbol] | CED83891 |
| X₅₆₅₈ | M6 | 69 | 1084848 | A | G | [symbol] | CED83891 |
| X₅₆₅₉ | M6 | 69 | 1084866 | A | G | [symbol] | CED83891 |
| X₅₆₆₀ | M6 | 69 | 1084931 | C | T | [symbol] | CED83891 |
| X₅₆₆₁ | M6 | 69 | 1102164 | C | T | [symbol] | CED83897 |
| X₅₆₆₂ | M6 | 69 | 1109785 | C | T | [symbol] | CED83899 |
| X₅₆₆₃ | M6 | 69 | 1109813 | A | G | [symbol] | CED83899 |
| X₃₆₆₄ | M6 | 69 | 1109818 | A | G | [symbol] | CED83899 |
| X₅₆₆₅ | M6 | 69 | 1109819 | T | A | [symbol] | CED83899 |
| X₅₆₆₆ | M6 | 69 | 1109823 | T | C | [symbol] | CED83899 |
| X₅₆₆₇ | M6 | 69 | 1109830 | A | G | [symbol] | CED83899 |
| X₅₆₆₈ | M6 | 69 | 1109831 | C | A | [symbol] | CED83899 |
| X₅₆₆₉ | M6 | 69 | 1109835 | A | G | [symbol] | CED83899 |
| X₅₆₇₀ | M6 | 69 | 1110546 | T | A | [symbol] | CED83899 |
| X₅₆₇₁ | M6 | 69 | 1111789 | C | T | [symbol] | CED83899 |
| X₅₆₇₂ | M6 | 69 | 1152012 | G | A | [symbol] | CED83915 |
| X₅₆₇₃ | M6 | 69 | 1179032 | G | A | [symbol] | CED83923 |
| X₅₆₇₄ | M6 | 69 | 1192333 | G | A | [symbol] | CED83928 |
| X₅₆₇₅ | M6 | 69 | 1205454 | C | T | [symbol] | CED83932 |
| X₅₆₇₆ | M6 | 69 | 1212727 | G | A | [symbol] | CED83933 |
| X₅₆₇₇ | M6 | 69 | 1218409 | G | A | [symbol] | CED83936 |
| X₅₆₇₈ | M6 | 69 | 1260087 | G | A | [symbol] | CED83949 |
| X₅₆₇₉ | M6 | 69 | 1342909 | G | A | [symbol] | CED83975 |
| X₅₆₈₀ | M6 | 69 | 1372486 | C | T | [symbol] | CED83987 |
| X₅₆₈₁ | M6 | 69 | 1377243 | C | T | [symbol] | CED83989 |
| X₅₆₈₂ | M6 | 69 | 1396045 | G | A | [symbol] | CED83994 |
| X₅₆₈₃ | M6 | 69 | 1411237 | C | T | [symbol] | CED84001 |
| X₅₆₈₄ | M6 | 69 | 1411421 | C | T | [symbol] | CED83999 |
| X₅₆₈₅ | M6 | 69 | 1412234 | G | A | [symbol] | CED83999 |
| X₅₆₈₆ | M6 | 69 | 1416914 | G | A | [symbol] | CED84002 |
| X₅₆₈₇ | M6 | 69 | 1421240 | C | T | [symbol] | CED84004 |
| X₅₆₈₈ | M6 | 69 | 1493453 | C | T | [symbol] | CED84026 |
| X₅₆₈₉ | M6 | 69 | 1577606 | C | T | [symbol] | CED84055 |
| X₅₆₉₀ | M6 | 69 | 1590302 | G | A | [symbol] | CED84059 |
| X₅₆₉₁ | M6 | 69 | 1642484 | C | T | [symbol] | CED84076 |
| X₅₆₉₂ | M6 | 69 | 1652288 | C | T | [symbol] | CED84077 |
| X₅₆₉₃ | M6 | 69 | 1674261 | C | T | [symbol] | CED84084 |
| X₅₆₉₄ | M6 | 69 | 1688555 | C | T | [symbol] | CED84088 |
| X₅₆₉₅ | M6 | 69 | 1694047 | C | T | [symbol] | CED84089 |
| X₅₆₉₆ | M6 | 69 | 1706650 | G | A | [symbol] | CED84095 |
| X₅₆₉₇ | M6 | 69 | 1714728 | G | A | [symbol] | CED84097 |
| X₅₆₉₈ | M6 | 69 | 1720246 | C | T | [symbol] | CED84101 |
| X₅₆₉₉ | M6 | 69 | 1790823 | C | T | [symbol] | CED84125 |
| X₅₇₀₀ | M6 | 69 | 1792597 | C | T | [symbol] | CED84126 |
| X₅₇₀₁ | M6 | 69 | 1800584 | G | A | [symbol] | CED84129 |
| X₅₇₀₂ | M6 | 69 | 1803813 | G | A | [symbol] | CED84128 |
| X₅₇₀₃ | M6 | 69 | 1854739 | G | A | [symbol] | CED84145 |
| X₅₇₀₄ | M6 | 69 | 1924621 | C | T | [symbol] | CED84172 |
| X₅₇₀₅ | M6 | 69 | 1960423 | G | A | [symbol] | CED84188 |
| X₅₇₀₆ | M6 | 69 | 1967529 | C | T | [symbol] | CED84189 |
| X₅₇₀₇ | M6 | 69 | 1968848 | A | G | [symbol] | CED84189 |
| X₅₇₀₈ | M6 | 69 | 1978129 | C | G | [symbol] | CED84193 |
| X₅₇₀₉ | M6 | 69 | 1978321 | A | G | [symbol] | CED84193 |
| X₅₇₁₀ | M6 | 69 | 2001469 | C | T | [symbol] | CED84197 |
| X₅₇₁₁ | M6 | 69 | 2089848 | A | G | [symbol] | CED84232 |
| X₅₇₁₂ | M6 | 69 | 2089870 | C | T | [symbol] | CED84232 |
| X₅₇₁₃ | M6 | 69 | 2089876 | C | A | [symbol] | CED84232 |
| X₅₇₁₄ | M6 | 69 | 2099165 | C | T | [symbol] | CED84233 |
| X₅₇₁₅ | M6 | 69 | 2099418 | C | T | [symbol] | CED84233 |
| X₅₇₁₆ | M6 | 69 | 2099421 | A | T | [symbol] | CED84233 |
| X₅₇₁₇ | M6 | 69 | 2099423 | G | T | [symbol] | CED84233 |
| X₅₇₁₈ | M6 | 69 | 2099452 | C | G | [symbol] | CED84233 |
| X₅₇₁₉ | M6 | 69 | 2101188 | T | C | [symbol] | CED84234 |
| X₅₇₂₀ | M6 | 69 | 2160207 | C | T | [symbol] | CED84255 |
| X₅₇₂₁ | M6 | 69 | 2180564 | C | T | [symbol] | CED84264 |
| X₅₇₂₂ | M6 | 69 | 2226113 | C | T | [symbol] | CED84272 |
| X₅₇₂₃ | M6 | 69 | 2231926 | G | A | [symbol] | CED84274 |
| X₅₇₂₄ | M6 | 69 | 2260222 | C | T | [symbol] | CED84282 |
| X₅₇₂₅ | M6 | 69 | 2438727 | C | T | [symbol] | CED84343 |
| X₅₇₂₆ | M6 | 69 | 2483433 | G | A | [symbol] | CED84352 |
| X₅₇₂₇ | M6 | 70 | 22 | T | C | [symbol] | |
| X₅₇₂₈ | M6 | 72 | 93 | T | C | [symbol] | |
| X₅₇₂₉ | M6 | 72 | 584 | G | A | [symbol] | |
| X₅₇₃₀ | M6 | 72 | 856 | C | T | [symbol] | |
| X₅₇₃₁ | M6 | 72 | 1163 | C | T | [symbol] | |
| X₅₇₃₂ | M6 | 77 | 34978 | G | T | [symbol] | CDZ96642 |
| X₅₇₃₃ | M6 | 77 | 39842 | G | A | [symbol] | CDZ96646 |
| X₅₇₃₄ | M6 | 77 | 84022 | G | A | [symbol] | CDZ96659 |
| X₅₇₃₅ | M6 | 78 | 1865 | C | T | [symbol] | CED84359 |
| X₅₇₃₆ | M6 | 78 | 6333 | G | A | [symbol] | CED84360 |
| X₅₇₃₇ | M6 | 78 | 89958 | C | T | [symbol] | CED84387 |
| X₅₇₃₈ | M6 | 78 | 100169 | G | A | [symbol] | CED84388 |
| X₅₇₃₉ | M6 | 78 | 109325 | G | A | [symbol] | CED84390 |
| X₅₇₄₀ | M6 | 78 | 127873 | G | A | [symbol] | CED84395 |
| X₅₇₄₁ | M6 | 78 | 139788 | C | A | [symbol] | CED84399 |
| X₅₇₄₂ | M6 | 78 | 238854 | C | T | [symbol] | CED84430 |
| X₅₇₄₃ | M6 | 78 | 263828 | T | C | [symbol] | CED84438 |
| X₅₇₄₄ | M6 | 78 | 276353 | T | C | [symbol] | CED84440 |
| X₅₇₄₅ | M6 | 78 | 276361 | T | C | [symbol] | CED84440 |
| X₅₇₄₆ | M6 | 78 | 276362 | C | T | [symbol] | CED84440 |
| X₅₇₄₇ | M6 | 78 | 281454 | C | A | [symbol] | CED84441 |
| X₅₇₄₈ | M6 | 78 | 292068 | C | T | [symbol] | CED84442 |
| X₅₇₄₉ | M6 | 78 | 292139 | A | T | [symbol] | CED84442 |
| X₅₇₅₀ | M6 | 78 | 298141 | C | G | [symbol] | CED84443 |
| X₅₇₅₁ | M6 | 78 | 299166 | G | A | [symbol] | CED84443 |
| X₅₇₅₂ | M6 | 78 | 299644 | G | A | [symbol] | CED84443 |
| X₅₇₅₃ | M6 | 78 | 302257 | G | A | [symbol] | CED84443 |
| X₅₇₅₄ | M6 | 78 | 311586 | C | T | [symbol] | CED84447 |
| X₅₇₅₅ | M6 | 78 | 404944 | G | A | [symbol] | CED84475 |
| X₅₇₅₆ | M6 | 78 | 404988 | G | A | [symbol] | CED84475 |
| X₅₇₅₇ | M6 | 78 | 487303 | C | T | [symbol] | CED84505 |
| X₅₇₅₈ | M6 | 78 | 517862 | G | A | [symbol] | CED84513 |
| X₅₇₅₉ | M6 | 78 | 611362 | C | T | [symbol] | CED84546 |
| X₅₇₆₀ | M6 | 78 | 646777 | C | T | [symbol] | CED84556 |
| X₅₇₆₁ | M6 | 78 | 653177 | C | T | [symbol] | CED84559 |
| X₅₇₆₂ | M6 | 78 | 658932 | G | A | [symbol] | CED84561 |
| X₅₇₆₃ | M6 | 78 | 708376 | C | T | [symbol] | CED84580 |
| X₅₇₆₄ | M6 | 78 | 763771 | G | A | [symbol] | CED84600 |
| X₅₇₆₅ | M6 | 78 | 774252 | G | A | [symbol] | CED84602 |
| X₅₇₆₆ | M6 | 78 | 853752 | G | A | [symbol] | CED84625 |
| X₅₇₆₇ | M6 | 78 | 952815 | G | A | [symbol] | CED84662 |
| X₅₇₆₈ | M6 | 78 | 952860 | G | A | [symbol] | CED84662 |
| X₅₇₆₉ | M6 | 78 | 976799 | C | T | [symbol] | CED84672 |
| X₅₇₇₀ | M6 | 78 | 988551 | G | A | [symbol] | CED84675 |
| X₅₇₇₁ | M6 | 78 | 1023381 | G | A | [symbol] | CED84688 |
| X₅₇₇₂ | M6 | 78 | 1086537 | C | T | [symbol] | CED84713 |
| X₅₇₇₃ | M6 | 78 | 1098373 | G | A | [symbol] | CED84716 |
| X₅₇₇₄ | M6 | 78 | 1112587 | C | T | [symbol] | CED84722 |
| X₅₇₇₅ | M6 | 78 | 1115511 | G | A | [symbol] | CED84723 |
| X₅₇₇₆ | M6 | 78 | 1155093 | C | T | [symbol] | CED84732 |
| X₅₇₇₇ | M6 | 78 | 1216573 | C | T | [symbol] | CED84752 |
| X₅₇₇₈ | M6 | 78 | 1266077 | G | A | [symbol] | CED84770 |
| X₅₇₇₉ | M6 | 78 | 1302286 | C | T | [symbol] | CED84782 |
| X₅₇₈₀ | M6 | 78 | 1317124 | G | A | [symbol] | CED84787 |
| X₅₇₈₁ | M6 | 78 | 1322274 | G | A | [symbol] | CED84790 |
| X₅₇₈₂ | M6 | 78 | 1344640 | C | T | [symbol] | CED84795 |
| X₅₇₈₃ | M6 | 78 | 1345792 | G | A | [symbol] | CED84797 |
| X₅₇₈₄ | M6 | 78 | 1406981 | C | T | [symbol] | CED84821 |
| X₅₇₈₅ | M6 | 78 | 1426722 | C | T | [symbol] | CED84829 |
| X₅₇₈₆ | M6 | 78 | 1463555 | G | A | [symbol] | CED84840 |
| X₅₇₈₇ | M6 | 78 | 1498179 | G | A | [symbol] | CED84852 |
| X₅₇₈₈ | M6 | 78 | 1520586 | C | T | [symbol] | CED84855 |
| X₅₇₈₉ | M6 | 78 | 1532973 | G | A | [symbol] | CED84857 |
| X₅₇₉₀ | M6 | 78 | 1549787 | C | T | [symbol] | CED84861 |
| X₅₇₉₁ | M6 | 78 | 1563246 | C | T | [symbol] | CED84865 |
| X₅₇₉₂ | M6 | 78 | 1568326 | C | T | [symbol] | CED84868 |
| X₅₇₉₃ | M6 | 78 | 1609850 | C | T | [symbol] | CED84880 |
| X₅₇₉₄ | M6 | 78 | 1642170 | G | A | [symbol] | CED84889 |
| X₅₇₉₅ | M6 | 78 | 1658512 | G | A | [symbol] | CED84896 |
| X₅₇₉₆ | M6 | 78 | 1660408 | G | A | [symbol] | CED84897 |
| X₅₇₉₇ | M6 | 78 | 1673601 | C | T | [symbol] | CED84903 |
| X₅₇₉₈ | M6 | 78 | 1705462 | G | A | [symbol] | CED84915 |
| X₅₇₉₉ | M6 | 78 | 1742796 | G | A | [symbol] | CED84926 |
| X₅₈₀₀ | M6 | 78 | 1745145 | A | G | [symbol] | CED84927 |
| X₅₈₀₁ | M6 | 78 | 1745162 | C | T | [symbol] | CED84927 |
| X₅₈₀₂ | M6 | 79 | 65 | G | A | [symbol] | CDZ96674 |
| X₅₈₀₃ | M6 | 79 | 6477 | T | C | [symbol] | CDZ96674- |
| | | | | | | | CDZ96675 |
| X₅₈₀₄ | M6 | 79 | 57804 | G | A | [symbol] | CDZ96687 |
| X₅₈₀₅ | M6 | 79 | 64133 | C | T | [symbol] | CDZ96688 |
| X₅₈₀₆ | M6 | 79 | 80215 | C | T | [symbol] | CDZ96695 |
| X₅₈₀₇ | M6 | 79 | 132683 | C | T | [symbol] | CDZ96713 |
| X₅₈₀₈ | M6 | 79 | 147578 | G | A | [symbol] | CDZ96719 |
| X₅₈₀₉ | M6 | 79 | 171814 | C | T | [symbol] | CDZ96727 |
| X₅₈₁₀ | M6 | 79 | 307021 | C | T | [symbol] | CDZ96777 |
| X₅₈₁₁ | M6 | 79 | 316481 | C | T | [symbol] | CDZ96779 |
| X₅₈₁₂ | M6 | 79 | 318883 | G | A | [symbol] | CDZ96779 |
| X₅₈₁₃ | M6 | 79 | 332458 | G | A | [symbol] | CDZ96785 |
| X₅₈₁₄ | M6 | 79 | 346823 | G | A | [symbol] | CDZ96789 |
| X₅₈₁₅ | M6 | 79 | 378247 | C | T | [symbol] | CDZ96800 |
| X₅₈₁₆ | M6 | 79 | 483118 | T | A | [symbol] | CDZ96836 |
| X₅₈₁₇ | M6 | 79 | 492523 | C | T | [symbol] | CDZ96842 |
| X₅₈₁₈ | M6 | 79 | 509666 | C | T | [symbol] | CDZ96848 |
| X₅₈₁₉ | M6 | 79 | 526910 | C | T | [symbol] | CDZ96854 |
| X₅₈₂₀ | M6 | 79 | 575411 | C | T | [symbol] | CDZ96868 |
| X₅₈₂₁ | M6 | 79 | 579674 | G | A | [symbol] | CDZ96871 |
| X₅₈₂₂ | M6 | 79 | 617057 | C | T | [symbol] | CDZ96882 |
| X₅₈₂₃ | M6 | 79 | 679140 | C | T | [symbol] | CDZ96904 |
| X₅₈₂₄ | M6 | 79 | 687842 | T | C | [symbol] | CDZ96908 |
| X₅₈₂₅ | M6 | 79 | 696174 | G | A | [symbol] | CDZ96911 |
| X₅₈₂₆ | M6 | 79 | 735053 | T | C | [symbol] | CDZ96928 |
| X₅₈₂₇ | M6 | 79 | 748083 | G | T | [symbol] | CDZ96929 |
| X₅₈₂₈ | M6 | 79 | 749308 | A | G | [symbol] | CDZ96930 |
| X₅₈₂₉ | M6 | 79 | 759195 | T | A | [symbol] | CDZ96931 |
| X₅₈₃₀ | M6 | 79 | 759403 | C | T | [symbol] | CDZ96931 |
| X₅₈₃₁ | M6 | 79 | 759589 | G | A | [symbol] | CDZ96932 |
| X₅₈₃₂ | M6 | 79 | 759659 | G | A | [symbol] | CDZ96931 |
| X₅₈₃₃ | M6 | 79 | 760430 | T | C | [symbol] | CDZ96932 |
| X₅₈₃₄ | M6 | 79 | 760891 | A | G | [symbol] | CDZ96932 |
| X₅₈₃₅ | M6 | 79 | 760988 | G | T | [symbol] | CDZ96932 |
| X₅₈₃₆ | M6 | 79 | 761056 | T | C | [symbol] | CDZ96932 |
| X₅₈₃₇ | M6 | 79 | 768537 | A | G | [symbol] | CDZ96933 |
| X₅₈₃₈ | M6 | 79 | 776301 | C | A | [symbol] | CDZ96934 |
| X₅₈₃₉ | M6 | 79 | 776304 | G | T | [symbol] | CDZ96934 |
| X₅₈₄₀ | M6 | 79 | 786064 | A | G | [symbol] | CDZ96934 |
| X₅₈₄₁ | M6 | 79 | 798799 | C | T | [symbol] | CDZ96939 |
| X₅₈₄₂ | M6 | 79 | 844705 | T | C | [symbol] | CDZ96954 |
| X₅₈₄₃ | M6 | 79 | 907000 | C | T | [symbol] | CDZ96976 |
| X₅₈₄₄ | M6 | 79 | 933234 | G | A | [symbol] | CDZ96987 |
| X₅₈₄₅ | M6 | 79 | 973493 | C | T | [symbol] | CDZ97000 |
| X₅₈₄₆ | M6 | 79 | 1000786 | A | G | [symbol] | CDZ97007 |
| X₅₈₄₇ | M6 | 79 | 1021855 | G | A | [symbol] | CDZ97016 |
| X₅₈₄₈ | M6 | 79 | 1044652 | G | A | [symbol] | CDZ97023 |
| X₅₈₄₉ | M6 | 79 | 1075960 | G | A | [symbol] | CDZ97034 |
| X₅₈₅₀ | M6 | 79 | 1107695 | C | T | [symbol] | CDZ97046 |
| X₅₈₅₁ | M6 | 79 | 1117040 | C | T | [symbol] | CDZ97047 |
| X₅₈₅₂ | M6 | 79 | 1123288 | C | T | [symbol] | CDZ97049 |
| X₅₈₅₃ | M6 | 79 | 1140763 | G | A | [symbol] | CDZ97053 |
| X₅₈₅₄ | M6 | 79 | 1167374 | C | T | [symbol] | CDZ97065 |
| X₅₈₅₅ | M6 | 79 | 1191202 | G | A | [symbol] | CDZ97070 |
| X₅₈₅₆ | M6 | 79 | 1211985 | G | A | [symbol] | CDZ97077 |
| X₅₈₅₇ | M6 | 79 | 1229129 | G | A | [symbol] | CDZ97086 |
| X₅₈₅₈ | M6 | 79 | 1231740 | C | T | [symbol] | CDZ97087 |
| X₅₈₅₉ | M6 | 79 | 1234552 | G | A | [symbol] | CDZ97087 |
| X₅₈₆₀ | M6 | 79 | 1318034 | C | T | [symbol] | CDZ97117 |
| X₅₈₆₁ | M6 | 79 | 1336766 | C | T | [symbol] | CDZ97121 |
| X₅₈₆₂ | M6 | 79 | 1344338 | G | A | [symbol] | CDZ97124 |
| X₅₈₆₃ | M6 | 79 | 1387800 | G | A | [symbol] | CDZ97135 |
| X₅₈₆₄ | M6 | 79 | 1394931 | A | T | [symbol] | CDZ97136 |
| X₅₈₆₅ | M6 | 79 | 1430954 | G | A | [symbol] | CDZ97152 |
| X₅₈₆₆ | M6 | 79 | 1472351 | C | T | [symbol] | CDZ97165 |
| X₅₈₆₇ | M6 | 79 | 1533781 | C | T | [symbol] | CDZ97185 |
| X₅₈₆₈ | M6 | 79 | 1540205 | C | T | [symbol] | CDZ97186 |
| X₅₈₆₉ | M6 | 79 | 1624415 | G | A | [symbol] | CDZ97217 |
| X₅₈₇₀ | M6 | 79 | 1658469 | C | T | [symbol] | CDZ97227 |
| X₅₈₇₁ | M6 | 79 | 1674448 | G | A | [symbol] | CDZ97228 |
| X₅₈₇₂ | M6 | 79 | 1704321 | G | A | [symbol] | CDZ97235 |
| X₅₈₇₃ | M6 | 79 | 1772244 | G | A | [symbol] | CDZ97258 |
| X₅₈₇₄ | M6 | 79 | 1797660 | C | T | [symbol] | CDZ97265 |
| X₅₈₇₅ | M6 | 79 | 1797676 | G | A | [symbol] | CDZ97265 |
| X₅₈₇₆ | M6 | 79 | 1842448 | G | A | [symbol] | CDZ97282 |
| X₅₈₇₇ | M6 | 79 | 1855037 | A | G | [symbol] | CDZ97285 |
| X₅₈₇₈ | M6 | 79 | 1859220 | A | G | [symbol] | CDZ97291 |
| X₅₈₇₉ | M6 | 79 | 1877329 | G | A | [symbol] | CDZ97295 |
| X₅₈₈₀ | M6 | 79 | 1912360 | G | A | [symbol] | CDZ97307 |
| X₅₈₈₁ | M6 | 79 | 1930688 | G | A | [symbol] | CDZ97315 |
| X₅₈₈₂ | M6 | 79 | 1938626 | A | C | [symbol] | CDZ97318 |
| X₅₈₈₃ | M6 | 79 | 2013361 | C | T | [symbol] | CDZ97344 |
| X₅₈₈₄ | M6 | 79 | 2052320 | C | T | [symbol] | CDZ97361 |
| X₅₈₈₅ | M6 | 79 | 2055184 | G | A | [symbol] | CDZ97361 |
| X₅₈₈₆ | M6 | 79 | 2113064 | G | A | [symbol] | CDZ97381 |
| X₅₈₈₇ | M6 | 79 | 2116476 | C | T | [symbol] | CDZ97380 |
| X₅₈₈₈ | M6 | 79 | 2121041 | G | A | [symbol] | CDZ97380 |
| X₅₈₈₉ | M6 | 79 | 2152573 | C | T | [symbol] | CDZ97392 |
| X₅₈₉₀ | M6 | 79 | 2188410 | G | A | [symbol] | CDZ97405 |
| X₅₈₉₁ | M6 | 79 | 2268540 | C | T | [symbol] | CDZ97435 |
| X₅₈₉₂ | M6 | 79 | 2304361 | G | A | [symbol] | CDZ97450 |
| X₅₈₉₃ | M6 | 79 | 2315869 | A | C | [symbol] | CDZ97452 |
| X₅₈₉₄ | M6 | 79 | 2323713 | C | T | [symbol] | CDZ97455 |
| X₅₈₉₅ | M6 | 79 | 2339881 | C | G | [symbol] | CDZ97460 |
| X₅₈₉₆ | M6 | 79 | 2340326 | G | A | [symbol] | CDZ97462 |
| X₅₈₉₇ | M6 | 81 | 210 | G | T | [symbol] | |
| X₅₈₉₈ | M6 | 85 | 512 | A | G | [symbol] | |
| X₅₈₉₉ | M6 | 85 | 516 | G | A | [symbol] | |
| X₅₉₀₀ | M6 | 85 | 521 | C | T | [symbol] | |
| X₅₉₀₁ | M6 | 85 | 522 | A | G | [symbol] | |
| X₅₉₀₂ | M6 | 88 | 119 | C | T | [symbol] | CDZ97464 |
| X₅₉₀₃ | M6 | 88 | 196 | G | A | [symbol] | CDZ97464 |
| X₅₉₀₄ | M6 | 88 | 898 | C | T | [symbol] | CDZ97464 |
| X₅₉₀₅ | M6 | 99 | 245 | G | A | [symbol] | |
| X₅₉₀₆ | M6 | 105 | 130 | T | A | [symbol] | CDZ97474 |
| X₅₉₀₇ | M6 | 107 | 278 | A | G | [symbol] | |
| X₅₉₀₈ | M6 | 109 | 396 | T | C | [symbol] | |
| X₅₉₀₉ | M6 | 113 | 264 | T | C | [symbol] | |
| X₅₉₁₀ | M6 | 118 | 396 | C | T | [symbol] | CDZ97478 |
| X₅₉₁₁ | M6 | 118 | 870 | G | A | [symbol] | CDZ97478 |
| X₅₉₁₂ | M6 | 118 | 1491 | G | A | [symbol] | CDZ97478 |
| X₅₉₁₃ | M6 | 121 | 318 | A | T | [symbol] | |
| X₅₉₁₄ | M6 | 121 | 1269 | A | T | [symbol] | |
| X₅₉₁₅ | M6 | 124 | 35211 | G | A | [symbol] | CDZ97488 |
| X₅₉₁₆ | M6 | 124 | 69730 | G | A | [symbol] | CDZ97501 |
| X₅₉₁₇ | M6 | 124 | 72117 | G | A | [symbol] | CDZ97501 |
| X₅₉₁₈ | M6 | 124 | 72760 | G | A | [symbol] | CDZ97501 |
| X₅₉₁₉ | M6 | 124 | 80266 | C | T | [symbol] | CDZ97504 |
| X₅₉₂₀ | M6 | 124 | 94611 | G | A | [symbol] | CDZ97507 |
| X₅₉₂₁ | M6 | 124 | 96777 | C | T | [symbol] | CDZ97508 |
| X₅₉₂₂ | M6 | 124 | 156052 | G | A | [symbol] | CDZ97524 |
| X₅₉₂₃ | M6 | 124 | 179336 | C | T | [symbol] | CDZ97533 |
| X₅₉₂₄ | M6 | 124 | 206541 | G | A | [symbol] | CDZ97541 |
| X₅₉₂₅ | M6 | 124 | 222249 | C | T | [symbol] | CDZ97547 |
| X₅₉₂₆ | M6 | 125 | 16379 | G | A | [symbol] | CDZ97565 |
| X₅₉₂₇ | M6 | 125 | 20772 | G | A | [symbol] | CDZ97566 |
| X₅₉₂₈ | M6 | 125 | 35864 | T | C | [symbol] | CDZ97573 |
| X₅₉₂₉ | M6 | 129 | 299 | G | A | [symbol] | CDZ97577 |
| X₅₉₃₀ | M6 | 129 | 433 | G | A | [symbol] | CDZ97577 |
| X₅₉₃₁ | M6 | 129 | 856 | G | A | [symbol] | CDZ97577 |
| X₅₉₃₂ | M6 | 129 | 1013 | C | T | [symbol] | CDZ97577 |
| X₅₉₃₃ | M6 | 130 | 674 | C | T | [symbol] | CDZ97578 |
| X₅₉₃₄ | M6 | 138 | 123 | G | T | [symbol] | |
| X₅₉₃₅ | M6 | 141 | 5649 | C | T | [symbol] | CDZ97582 |
| X₅₉₃₆ | M6 | 141 | 14167 | G | A | [symbol] | CDZ97586 |
| X₅₉₃₇ | M6 | 141 | 15764 | C | T | [symbol] | CDZ97587 |
| X₅₉₃₈ | M6 | 141 | 29260 | G | A | [symbol] | CDZ97594 |
| X₅₉₃₉ | M6 | 145 | 296 | A | G | [symbol] | |
| X₅₀₄₀ | M6 | 149 | 1663 | A | G | [symbol] | CDZ97598 |
| X₅₉₄₁ | M6 | 149 | 2076 | T | G | [symbol] | CDZ97598 |
| X₅₉₄₂ | M6 | 149 | 2453 | C | T | [symbol] | CDZ97598 |
| X₅₉₄₃ | M6 | 159 | 106 | A | C | [symbol] | |
| X₅₉₄₄ | M6 | 162 | 47699 | G | A | [symbol] | CDZ97619 |
| X₅₉₄₅ | M6 | 162 | 70405 | A | G | [symbol] | CDZ97625 |
| X₅₉₄₆ | M6 | 162 | 209584 | C | T | [symbol] | CDZ97670 |
| X₅₉₄₇ | M6 | 162 | 214099 | G | A | [symbol] | CDZ97671 |
| X₅₉₄₈ | M6 | 162 | 218351 | G | A | [symbol] | CDZ97671 |
| X₅₉₄₉ | M6 | 162 | 223407 | C | T | [symbol] | CDZ97674 |
| X₅₉₅₀ | M6 | 162 | 308369 | T | C | [symbol] | CDZ97702 |
| X₅₉₅₁ | M6 | 162 | 320785 | G | C | [symbol] | CDZ97703 |
| X₅₉₅₂ | M6 | 162 | 320786 | T | G | [symbol] | CDZ97703 |
| X₅₉₅₃ | M6 | 162 | 321070 | C | T | [symbol] | CDZ97703 |
| X₅₉₅₄ | M6 | 162 | 321073 | A | G | [symbol] | CDZ97703 |
| X₅₉₅₅ | M6 | 162 | 321460 | C | G | [symbol] | CDZ97703 |
| X₅₉₅₆ | M6 | 162 | 321463 | A | C | [symbol] | CDZ97703 |
| X₅₉₅₇ | M6 | 162 | 323651 | T | C | [symbol] | CDZ97703 |
| X₅₉₅₈ | M6 | 162 | 323735 | A | G | [symbol] | CDZ97703 |
| X₅₉₅₉ | M6 | 162 | 324114 | T | C | [symbol] | CDZ97703 |
| X₅₉₆₀ | M6 | 162 | 324139 | C | T | [symbol] | CDZ97703 |
| X₅₉₆₁ | M6 | 162 | 324568 | A | G | [symbol] | CDZ97703 |
| X₅₉₆₂ | M6 | 162 | 360320 | G | A | [symbol] | CDZ97714 |
| X₅₉₆₃ | M6 | 162 | 387700 | C | T | [symbol] | CDZ97725 |
| X₅₉₆₄ | M6 | 162 | 408274 | C | T | [symbol] | CDZ97733 |
| X₅₉₆₅ | M6 | 162 | 423180 | C | T | [symbol] | CDZ97740 |
| X₅₉₆₆ | M6 | 162 | 424391 | C | T | [symbol] | CDZ97740 |
| X₅₉₆₇ | M6 | 162 | 444123 | T | C | [symbol] | CDZ97748 |
| X₅₉₆₈ | M6 | 162 | 467207 | C | T | [symbol] | CDZ97755 |
| X₅₉₆₉ | M6 | 162 | 476267 | C | T | [symbol] | CDZ97760 |
| X₅₉₇₀ | M6 | 162 | 481093 | G | A | [symbol] | CDZ97761 |
| X₅₉₇₁ | M6 | 162 | 491906 | G | A | [symbol] | CDZ97764 |
| X₅₉₇₂ | M6 | 162 | 492101 | G | A | [symbol] | CDZ97764 |
| X₅₉₇₃ | M6 | 162 | 501886 | C | T | [symbol] | CDZ97769 |
| X₅₉₇₄ | M6 | 162 | 507949 | G | A | [symbol] | CDZ97771 |
| X₅₉₇₅ | M6 | 162 | 521996 | G | C | [symbol] | CDZ97777 |
| X₅₉₇₆ | M6 | 162 | 618658 | C | T | [symbol] | CDZ97808 |
| X₅₉₇₇ | M6 | 162 | 632311 | G | A | [symbol] | CDZ97814 |
| X₅₉₇₈ | M6 | 162 | 633321 | G | A | [symbol] | CDZ97814 |
| X₅₉₇₉ | M6 | 162 | 633633 | G | A | [symbol] | CDZ97814 |
| X₅₉₈₀ | M6 | 162 | 638083 | C | T | [symbol] | CDZ97814 |
| X₅₉₈₁ | M6 | 162 | 640005 | C | T | [symbol] | CDZ97815 |
| X₅₉₈₂ | M6 | 162 | 680645 | G | A | [symbol] | CDZ97826 |
| X₅₉₈₃ | M6 | 162 | 716116 | C | T | [symbol] | CDZ97836 |
| X₅₉₈₄ | M6 | 162 | 738479 | G | A | [symbol] | CDZ97842 |
| X₅₉₈₅ | M6 | 162 | 738675 | G | A | [symbol] | CDZ97842 |
| X₅₉₈₆ | M6 | 162 | 738686 | C | T | [symbol] | CDZ97842 |
| X₅₉₈₇ | M6 | 162 | 808830 | G | A | [symbol] | CDZ97869 |
| X₅₉₈₈ | M6 | 162 | 825209 | G | A | [symbol] | CDZ97877 |
| X₅₉₈₉ | M6 | 162 | 871911 | C | T | [symbol] | CDZ97890 |
| X₅₉₉₀ | M6 | 162 | 914850 | C | T | [symbol] | CDZ97904 |
| X₅₉₉₁ | M6 | 162 | 972973 | G | A | [symbol] | CDZ97925 |
| X₅₉₉₂ | M6 | 163 | 225 | G | A | [symbol] | |
| X₅₉₉₃ | M6 | 163 | 727 | A | G | [symbol] | |
| X₅₉₉₄ | M6 | 175 | 120 | G | C | [symbol] | |
| X₅₉₉₅ | M6 | 185 | 21 | C | T | [symbol] | CDZ97957 |
| X₅₉₉₆ | M6 | 189 | 1306 | C | T | [symbol] | CDZ97961 |
| X₅₉₉₇ | M6 | 189 | 64473 | G | A | [symbol] | CDZ97979 |
| X₅₉₉₈ | M6 | 189 | 126573 | G | A | [symbol] | CDZ97996 |
| X₅₉₉₉ | M6 | 189 | 194647 | A | G | [symbol] | CDZ98020 |
| X₆₀₀₀ | M6 | 189 | 194665 | A | G | [symbol] | CDZ98020 |
| X₆₀₀₁ | M6 | 189 | 211865 | C | T | [symbol] | CDZ98028 |
| X₆₀₀₂ | M6 | 189 | 212649 | C | T | [symbol] | CDZ98028 |
| X₆₀₀₃ | M6 | 189 | 323680 | C | T | [symbol] | CDZ98063 |
| X₆₀₀₄ | M6 | 189 | 344057 | C | T | [symbol] | CDZ98068 |
| X₆₀₀₅ | M6 | 189 | 371247 | G | A | [symbol] | CDZ98078 |
| X₆₀₀₆ | M6 | 189 | 394551 | C | T | [symbol] | CDZ98085 |
| X₆₀₀₇ | M6 | 191 | 342 | A | G | [symbol] | CDZ98107 |
| X₆₀₀₈ | M6 | 191 | 347 | C | T | [symbol] | CDZ98107 |
| X₆₀₀₉ | M6 | 191 | 427 | C | T | [symbol] | CDZ98107 |
| X₆₀₁₀ | M6 | 191 | 451 | C | T | [symbol] | CDZ98107 |
| X₆₀₁₁ | M6 | 191 | 487 | A | G | [symbol] | CDZ98107 |
| X₆₀₁₂ | M6 | 197 | 624 | C | T | [symbol] | CDZ98111 |
| X₆₀₁₃ | M6 | 198 | 202 | C | A | [symbol] | |
| X₆₀₁₄ | M6 | 198 | 203 | G | A | [symbol] | |
| X₆₀₁₅ | M6 | 198 | 238 | C | T | [symbol] | |
| X₆₀₁₆ | M6 | 198 | 265 | C | T | [symbol] | |
| X₆₀₁₇ | M6 | 199 | 688 | C | T | [symbol] | |
| X₆₀₁₈ | M6 | 199 | 911 | C | T | [symbol] | |
| X₆₀₁₉ | M6 | 199 | 971 | C | T | [symbol] | |
| X₆₀₂₀ | M6 | 199 | 1218 | C | T | [symbol] | |
| X₆₀₂₁ | M6 | 202 | 171 | C | T | [symbol] | |
| X₆₀₂₂ | M6 | 205 | 360 | C | T | [symbol] | CDZ98112 |
| X₆₀₂₃ | M6 | 205 | 981 | C | T | [symbol] | CDZ98112 |
| X₆₀₂₄ | M6 | 206 | 406 | G | A | [symbol] | |
| X₆₀₂₅ | M6 | 208 | 117 | G | A | [symbol] | |
| X₆₀₂₆ | M6 | 208 | 476 | G | A | [symbol] | |
| X₆₀₂₇ | M6 | 208 | 616 | G | A | [symbol] | |
| X₆₀₂₈ | M6 | 208 | 935 | G | A | [symbol] | |
| X₆₀₂₉ | M6 | 209 | 312 | C | T | [symbol] | CDZ98113 |
| X₆₀₃₀ | M6 | 223 | 93 | G | A | [symbol] | CDZ98118 |
| X₆₀₃₁ | M6 | 223 | 102 | A | G | [symbol] | CDZ98118 |
| X₆₀₃₂ | M6 | 223 | 133 | A | G | [symbol] | CDZ98118 |
| X₆₀₃₃ | M6 | 223 | 150 | T | C | [symbol] | CDZ98118 |
| X₆₀₃₄ | M6 | 223 | 155 | A | C | [symbol] | CDZ98118 |
| X₆₀₃₅ | M6 | 223 | 158 | T | C | [symbol] | CDZ98118 |
| X₆₀₃₆ | M6 | 223 | 160 | C | T | [symbol] | CDZ98118 |
| X₆₀₃₇ | M6 | 223 | 175 | G | A | [symbol] | CDZ98118 |
| X₆₀₃₈ | M6 | 223 | 177 | A | G | [symbol] | CDZ98118 |
| X₆₀₃₉ | M6 | 223 | 188 | A | G | [symbol] | CDZ98118 |
| X₆₀₄₀ | M6 | 223 | 192 | C | A | [symbol] | CDZ98118 |
| X₆₀₄₁ | M6 | 223 | 194 | T | C | [symbol] | CDZ98118 |
| X₆₀₄₂ | M6 | 223 | 195 | T | G | [symbol] | CDZ98118 |
| X₆₀₄₃ | M6 | 223 | 197 | T | C | [symbol] | CDZ98118 |
| X₆₀₄₄ | M6 | 223 | 202 | G | C | [symbol] | CDZ98118 |
| X₆₀₄₅ | M6 | 225 | 317 | C | T | [symbol] | |
| X₆₀₄₆ | M6 | 227 | 202 | G | A | [symbol] | |
| X₆₀₄₇ | M6 | 232 | 340 | G | A | [symbol] | CDZ98119 |
| X₆₀₄₈ | M6 | 232 | 1379 | C | T | [symbol] | CDZ98119 |
| X₆₀₄₉ | M6 | 232 | 1521 | C | T | [symbol] | CDZ98119 |
| X₆₀₅₀ | M6 | 232 | 1662 | G | A | [symbol] | CDZ98119 |
| X₆₀₅₁ | M6 | 232 | 1761 | C | T | [symbol] | CDZ98119 |
| X₆₀₅₂ | M6 | 232 | 2027 | C | T | [symbol] | CDZ98119 |
| X₆₀₅₃ | M6 | 232 | 3060 | C | T | [symbol] | CDZ98120 |
| X₆₀₅₄ | M6 | 232 | 3284 | C | T | [symbol] | CDZ98119 |
| X₆₀₅₅ | M6 | 232 | 4437 | G | A | [symbol] | CDZ98121 |
| X₆₀₅₆ | M6 | 232 | 4561 | C | T | [symbol] | CDZ98120 |
| X₆₀₅₇ | M6 | 242 | 7334 | C | T | [symbol] | CDZ98125 |
| X₆₀₅₈ | M6 | 242 | 41710 | G | A | [symbol] | CDZ98131 |
| X₆₀₅₉ | M6 | 242 | 50262 | G | A | [symbol] | CDZ98135 |
| X₆₀₆₀ | M6 | 242 | 84132 | G | A | [symbol] | CDZ98146 |
| X₆₀₆₁ | M6 | 242 | 88037 | G | A | [symbol] | CDZ98147 |
| X₆₀₆₂ | M6 | 242 | 107587 | G | A | [symbol] | CDZ98153 |
| X₆₀₆₃ | M6 | 242 | 112244 | G | A | [symbol] | CDZ98154 |
| X₆₀₆₄ | M6 | 242 | 149113 | C | T | [symbol] | CDZ98165 |
| X₆₀₆₅ | M6 | 242 | 213986 | G | A | [symbol] | CDZ98185 |
| X₆₀₆₆ | M6 | 242 | 227271 | C | T | [symbol] | CDZ98191 |
| X₆₀₆₇ | M6 | 242 | 233088 | G | A | [symbol] | CDZ98193 |
| X₆₀₆₈ | M6 | 242 | 233141 | G | A | [symbol] | CDZ98193 |
| X₆₀₆₉ | M6 | 242 | 310353 | G | A | [symbol] | CDZ98216 |
| X₆₀₇₀ | M6 | 242 | 342570 | C | T | [symbol] | CDZ98229 |
| X₆₀₇₁ | M6 | 242 | 357209 | G | A | [symbol] | CDZ98233 |
| X₆₀₇₂ | M6 | 242 | 374897 | G | A | [symbol] | CDZ98238 |
| X₆₀₇₃ | M6 | 242 | 376713 | G | A | [symbol] | CDZ98238 |
| X₆₀₇₄ | M6 | 242 | 379812 | C | T | [symbol] | CDZ98238 |
| X₆₀₇₅ | M6 | 242 | 404132 | G | A | [symbol] | CDZ98248 |
| X₆₀₇₆ | M6 | 242 | 405825 | G | A | [symbol] | CDZ98250 |
| X₆₀₇₇ | M6 | 242 | 412676 | G | A | [symbol] | CDZ98249 |
| X₆₀₇₈ | M6 | 242 | 437941 | G | A | [symbol] | CDZ98258 |
| X₆₀₇₉ | M6 | 242 | 454709 | C | T | [symbol] | CDZ98263 |
| X₆₀₈₀ | M6 | 242 | 485936 | G | A | [symbol] | CDZ98270 |
| X₆₀₈₁ | M6 | 242 | 492389 | G | A | [symbol] | CDZ98274 |
| X₆₀₈₂ | M6 | 242 | 514780 | C | T | [symbol] | CDZ98282 |
| X₆₀₈₃ | M6 | 242 | 528340 | G | A | [symbol] | CDZ98288 |
| X₆₀₈₄ | M6 | 242 | 530144 | G | A | [symbol] | CDZ98288 |
| X₆₀₈₅ | M6 | 242 | 542663 | G | A | [symbol] | CDZ98290 |
| X₆₀₈₆ | M6 | 242 | 548679 | C | T | [symbol] | CDZ98292 |
| X₆₀₈₇ | M6 | 242 | 559999 | G | A | [symbol] | CDZ98297 |
| X₆₀₈₈ | M6 | 242 | 602456 | G | A | [symbol] | CDZ98308 |
| X₆₀₈₉ | M6 | 242 | 602531 | G | A | [symbol] | CDZ98308 |
| X₆₀₉₀ | M6 | 242 | 604704 | C | T | [symbol] | CDZ98309 |
| X₆₀₉₁ | M6 | 242 | 628488 | G | A | [symbol] | CDZ98317 |
| X₆₀₉₂ | M6 | 248 | 99 | A | G | [symbol] | |
| X₆₀₉₃ | M6 | 249 | 206 | G | A | [symbol] | CED84929 |
| X₆₀₉₄ | M6 | 249 | 5307 | C | T | [symbol] | CED84929 |
| X₆₀₉₅ | M6 | 249 | 52397 | G | A | [symbol] | CED84947 |
| X₆₀₉₆ | M6 | 249 | 72016 | C | T | [symbol] | CED84950 |
| X₆₀₉₇ | M6 | 249 | 157550 | C | T | [symbol] | CED84983 |
| X₆₀₉₈ | M6 | 249 | 191304 | C | T | [symbol] | CED84988 |
| X₆₀₉₉ | M6 | 249 | 231983 | C | T | [symbol] | CED85003 |
| X₆₁₀₀ | M6 | 249 | 238318 | C | T | [symbol] | CED85003 |
| X₆₁₀₁ | M6 | 249 | 239775 | G | A | [symbol] | CED85004 |
| X₆₁₀₂ | M6 | 249 | 253494 | C | T | [symbol] | CED85008 |
| X₆₁₀₃ | M6 | 249 | 272075 | C | T | [symbol] | CED85013 |
| X₆₁₀₄ | M6 | 249 | 294853 | C | T | [symbol] | CED85019 |
| X₆₁₀₅ | M6 | 249 | 305975 | G | A | [symbol] | CED85023 |
| X₆₁₀₆ | M6 | 249 | 342990 | G | A | [symbol] | CED85036 |
| X₆₁₀₇ | M6 | 249 | 371117 | G | A | [symbol] | CED85047 |
| X₆₁₀₈ | M6 | 249 | 371535 | G | A | [symbol] | CED85048 |
| X₆₁₀₉ | M6 | 249 | 377814 | G | A | [symbol] | CED85051 |
| X₆₁₁₀ | M6 | 249 | 436175 | G | A | [symbol] | CED85073 |
| X₆₁₁₁ | M6 | 249 | 485782 | C | T | [symbol] | CED85089 |
| X₆₁₁₂ | M6 | 249 | 500195 | C | T | [symbol] | CED85093 |
| X₆₁₁₃ | M6 | 249 | 501310 | C | T | [symbol] | CED85093 |
| X₆₁₁₄ | M6 | 249 | 501510 | T | C | [symbol] | CED85093 |
| X₆₁₁₅ | M6 | 249 | 532943 | A | G | [symbol] | CED85099 |
| X₆₁₁₆ | M6 | 249 | 598579 | C | T | [symbol] | CED85125 |
| X₆₁₁₇ | M6 | 249 | 627104 | C | T | [symbol] | CED85133 |
| X₆₁₁₈ | M6 | 249 | 637944 | G | A | [symbol] | CED85135 |
| X₆₁₁₉ | M6 | 249 | 654772 | C | T | [symbol] | CED85141 |
| X₆₁₂₀ | M6 | 249 | 660408 | G | A | [symbol] | CED85144 |
| X₆₁₂₁ | M6 | 249 | 665383 | G | A | [symbol] | CED85144 |
| X₆₁₂₂ | M6 | 249 | 709695 | G | T | [symbol] | CED85164 |
| X₆₁₂₃ | M6 | 249 | 743970 | G | A | [symbol] | CED85171 |
| X₆₁₂₄ | M6 | 249 | 751427 | G | A | [symbol] | CED85174 |
| X₆₁₂₅ | M6 | 249 | 794731 | C | T | [symbol] | CED85186 |
| X₆₁₂₆ | M6 | 249 | 803215 | C | T | [symbol] | CED85186 |
| X₆₁₂₇ | M6 | 249 | 854220 | G | A | [symbol] | CED85201 |
| X₆₁₂₈ | M6 | 249 | 886063 | G | A | [symbol] | CED85208 |
| X₆₁₂₉ | M6 | 249 | 911274 | C | T | [symbol] | CED85218 |
| X₆₁₃₀ | M6 | 249 | 932606 | C | T | [symbol] | CED85223 |
| X₆₁₃₁ | M6 | 249 | 934305 | G | A | [symbol] | CED85225 |
| X₆₁₃₂ | M6 | 249 | 945434 | C | T | [symbol] | CED85227 |
| X₆₁₃₃ | M6 | 249 | 945727 | C | T | [symbol] | CED85227 |
| X₆₁₃₄ | M6 | 249 | 969333 | G | T | [symbol] | CED85238 |
| X₆₁₃₅ | M6 | 249 | 977170 | C | T | [symbol] | CED85242 |
| X₆₁₃₆ | M6 | 249 | 989520 | C | T | [symbol] | CED85243 |
| X₆₁₃₇ | M6 | 249 | 1014098 | C | T | [symbol] | CED85252 |
| X₆₁₃₈ | M6 | 249 | 1014926 | C | T | [symbol] | CED85252 |
| X₆₁₃₉ | M6 | 249 | 1050123 | C | T | [symbol] | CED85262 |
| X₆₁₄₀ | M6 | 249 | 1076166 | C | T | [symbol] | CED85270 |
| X₆₁₄₁ | M6 | 249 | 1076333 | G | T | [symbol] | CED85270 |
| X₆₁₄₂ | M6 | 249 | 1088841 | T | C | [symbol] | CED85274 |
| X₆₁₄₃ | M6 | 249 | 1127638 | C | T | [symbol] | CED85286 |
| X₆₁₄₄ | M6 | 249 | 1144309 | G | A | [symbol] | CED85291 |
| X₆₁₄₅ | M6 | 249 | 1176977 | G | A | [symbol] | CED85303 |
| X₆₁₄₆ | M6 | 249 | 1195367 | G | A | [symbol] | CED85307 |
| X₆₁₄₇ | M6 | 249 | 1230068 | C | T | [symbol] | CED85319 |
| X₆₁₄₈ | M6 | 249 | 1234239 | C | T | [symbol] | CED85319 |
| X₆₁₄₉ | M6 | 249 | 1245242 | G | A | [symbol] | CED85322 |
| X₆₁₅₀ | M6 | 249 | 1263893 | C | T | [symbol] | CED85327 |
| X₆₁₅₁ | M6 | 249 | 1299472 | C | T | [symbol] | CED85343 |
| X₆₁₅₂ | M6 | 249 | 1305184 | G | A | [symbol] | CED85345 |
| X₆₁₅₃ | M6 | 249 | 1331428 | G | A | [symbol] | CED85352 |
| X₆₁₅₄ | M6 | 249 | 1457529 | G | A | [symbol] | CED85398 |
| X₆₁₅₅ | M6 | 249 | 1475590 | G | A | [symbol] | CED85404 |
| X₆₁₅₆ | M6 | 249 | 1498029 | T | C | [symbol] | CED85415 |
| X₆₁₅₇ | M6 | 249 | 1498823 | T | C | [symbol] | CED85414 |
| X₆₁₅₈ | M6 | 249 | 1498829 | C | T | [symbol] | CED85414 |
| X₆₁₅₉ | M6 | 249 | 1502631 | G | T | [symbol] | CED85416 |
| X₆₁₆₀ | M6 | 249 | 1502641 | T | A | [symbol] | CED85416 |
| X₆₁₆₁ | M6 | 249 | 1505138 | G | A | [symbol] | CED85416 |
| X₆₁₆₂ | M6 | 249 | 1505634 | T | G | [symbol] | CED85416 |
| X₆₁₆₃ | M6 | 249 | 1518591 | T | A | [symbol] | CED85419 |
| X₆₁₆₄ | M6 | 249 | 1519431 | A | G | [symbol] | CED85419 |
| X₆₁₆₅ | M6 | 249 | 1520073 | T | C | [symbol] | CED85420 |
| X₆₁₆₆ | M6 | 249 | 1520089 | C | T | [symbol] | CED85420 |
| X₆₁₆₇ | M6 | 249 | 1520169 | C | T | [symbol] | CED85420 |
| X₆₁₆₈ | M6 | 249 | 1520420 | G | C | [symbol] | CED85420 |
| X₆₁₆₉ | M6 | 249 | 1531574 | C | T | [symbol] | CED85423 |
| X₆₁₇₀ | M6 | 249 | 1535511 | G | A | [symbol] | CED85423 |
| X₆₁₇₁ | M6 | 249 | 1567124 | G | A | [symbol] | CED85432 |
| X₆₁₇₂ | M6 | 249 | 1585540 | T | C | [symbol] | CED85439 |
| X₆₁₇₃ | M6 | 249 | 1599907 | C | T | [symbol] | CED85444 |
| X₆₁₇₄ | M6 | 249 | 1619036 | G | A | [symbol] | CED85450 |
| X₆₁₇₅ | M6 | 249 | 1636931 | G | A | [symbol] | CED85455 |
| X₆₁₇₆ | M6 | 249 | 1653615 | T | G | [symbol] | CED85457 |
| X₆₁₇₇ | M6 | 249 | 1653962 | C | T | [symbol] | CED85458 |
| X₆₁₇₈ | M6 | 249 | 1666185 | C | T | [symbol] | CED85460 |
| X₆₁₇₉ | M6 | 249 | 1726777 | C | T | [symbol] | CED85474 |
| X₆₁₈₀ | M6 | 249 | 1729264 | C | T | [symbol] | CED85476 |
| X₆₁₈₁ | M6 | 249 | 1732115 | G | A | [symbol] | CED85476 |
| X₆₁₈₂ | M6 | 249 | 1734722 | G | A | [symbol] | CED85477 |
| X₆₁₈₃ | M6 | 249 | 1768844 | C | T | [symbol] | CED85489 |
| X₆₁₈₄ | M6 | 249 | 1780551 | A | G | [symbol] | CED85493 |
| X₆₁₈₅ | M6 | 249 | 1780569 | A | G | [symbol] | CED85493 |
| X₆₁₈₆ | M6 | 249 | 1782994 | G | A | [symbol] | CED85493 |
| X₆₁₈₇ | M6 | 249 | 1801965 | G | A | [symbol] | CED85501 |
| X₆₁₈₈ | M6 | 249 | 1803575 | C | T | [symbol] | CED85502 |
| X₆₁₈₉ | M6 | 249 | 1804835 | G | A | [symbol] | CED85502 |
| X₆₁₉₀ | M6 | 249 | 1880762 | G | A | [symbol] | CED85525 |
| X₆₁₉₁ | M6 | 249 | 1944026 | G | A | [symbol] | CED85545 |
| X₆₁₉₂ | M6 | 249 | 1988505 | C | T | [symbol] | CED85560 |
| X₆₁₉₃ | M6 | 249 | 2015272 | G | A | [symbol] | CED85567 |
| X₆₁₉₄ | M6 | 249 | 2027477 | G | A | [symbol] | CED85573 |
| X₆₁₉₅ | M6 | 249 | 2047401 | C | T | [symbol] | CED85581 |
| X₆₁₉₆ | M6 | 249 | 2054064 | C | T | [symbol] | CED85583 |
| X₆₁₉₇ | M6 | 249 | 2063221 | G | A | [symbol] | CED85586 |
| X₆₁₉₈ | M6 | 249 | 2110956 | C | T | [symbol] | CED85603 |
| X₆₁₉₉ | M6 | 249 | 2131353 | C | T | [symbol] | CED85609 |
| X₆₂₀₀ | M6 | 249 | 2163304 | G | A | [symbol] | CED85618 |
| X₆₂₀₁ | M6 | 249 | 2198205 | G | A | [symbol] | CED85628 |
| X₆₂₀₂ | M6 | 249 | 2223626 | G | A | [symbol] | CED85633 |
| X₆₂₀₃ | M6 | 249 | 2255547 | G | C | [symbol] | CED85640 |
| X₆₂₀₄ | M6 | 252 | 512 | C | T | [symbol] | |
| X₆₂₀₅ | M6 | 252 | 524 | C | T | [symbol] | |
| X₆₂₀₆ | M6 | 258 | 836 | A | G | [symbol] | |
| X₆₂₀₇ | M6 | 258 | 907 | A | G | [symbol] | |
| X₆₂₀₈ | M6 | 258 | 1139 | A | T | [symbol] | |
| X₆₂₀₉ | M6 | 259 | 91 | G | C | [symbol] | |
| X₆₂₁₀ | M6 | 260 | 156 | G | A | [symbol] | |
| X₆₂₁₁ | M6 | 262 | 4049 | G | A | [symbol] | CDZ98330 |
| X₆₂₁₂ | M6 | 262 | 12289 | C | T | [symbol] | CDZ98333 |
| X₆₂₁₃ | M6 | 262 | 30347 | G | A | [symbol] | CDZ98338 |
| X₆₂₁₄ | M6 | 262 | 30359 | G | A | [symbol] | CDZ98338 |
| X₆₂₁₅ | M6 | 262 | 39621 | C | T | [symbol] | CDZ98342 |
| X₆₂₁₆ | M6 | 262 | 81226 | G | A | [symbol] | CDZ98358 |
| X₆₂₁₇ | M6 | 262 | 109833 | C | T | [symbol] | CDZ98369 |
| X₆₂₁₈ | M6 | 262 | 139442 | C | T | [symbol] | CDZ98375 |
| X₆₂₁₉ | M6 | 262 | 148151 | C | T | [symbol] | CDZ98375 |
| X₆₂₂₀ | M6 | 262 | 164438 | G | A | [symbol] | CDZ98383 |
| X₆₂₂₁ | M6 | 262 | 175142 | G | A | [symbol] | CDZ98388 |
| X₆₂₂₂ | M6 | 262 | 201531 | G | A | [symbol] | CDZ98396 |
| X₆₂₂₃ | M6 | 262 | 242371 | C | T | [symbol] | CDZ98411 |
| X₆₂₂₄ | M6 | 262 | 314812 | T | C | [symbol] | CDZ98435 |
| X₆₂₂₅ | M6 | 262 | 319302 | G | A | [symbol] | CDZ98436 |
| X₆₂₂₆ | M6 | 262 | 345538 | C | T | [symbol] | CDZ98448 |
| X₆₂₂₇ | M6 | 262 | 373766 | C | T | [symbol] | CDZ98456 |
| X₆₂₂₈ | M6 | 262 | 380519 | C | T | [symbol] | CDZ98457 |
| X₆₂₂₉ | M6 | 262 | 397539 | G | A | [symbol] | CDZ98463 |
| X₆₂₃₀ | M6 | 262 | 420102 | G | A | [symbol] | CDZ98468 |
| X₆₂₃₁ | M6 | 262 | 446956 | C | T | [symbol] | CDZ98477 |
| X₆₂₃₂ | M6 | 262 | 471054 | C | T | [symbol] | CDZ98487 |
| X₆₂₃₃ | M6 | 262 | 498239 | C | T | [symbol] | CDZ98496 |
| X₆₂₃₄ | M6 | 262 | 503840 | G | A | [symbol] | CDZ98499 |
| X₆₂₃₅ | M6 | 262 | 510643 | G | A | [symbol] | CDZ98502 |
| X₆₂₃₆ | M6 | 262 | 539428 | G | A | [symbol] | CDZ98514 |
| X₆₂₃₇ | M6 | 262 | 539452 | A | G | [symbol] | CDZ98514 |
| X₆₂₃₈ | M6 | 262 | 539470 | A | G | [symbol] | CDZ98514 |
| X₆₂₃₉ | M6 | 262 | 539588 | A | G | [symbol] | CDZ98514 |
| X₆₂₄₀ | M6 | 262 | 552414 | C | T | [symbol] | CDZ98518 |
| X₆₂₄₁ | M6 | 262 | 557280 | G | A | [symbol] | CDZ98518 |
| X₆₂₄₂ | M6 | 262 | 560713 | G | A | [symbol] | CDZ98519 |
| X₆₂₄₃ | M6 | 262 | 562795 | C | T | [symbol] | CDZ98521 |
| X₆₂₄₄ | M6 | 262 | 567591 | G | A | [symbol] | CDZ98524 |
| X₆₂₄₅ | M6 | 262 | 567801 | G | A | [symbol] | CDZ98524 |
| X₆₂₄₆ | M6 | 262 | 618198 | G | A | [symbol] | CDZ98538 |
| X₆₂₄₇ | M6 | 262 | 639962 | A | G | [symbol] | CDZ98544 |
| X₆₂₄₈ | M6 | 262 | 653920 | A | G | [symbol] | CDZ98545 |
| X₆₂₄₉ | M6 | 262 | 656031 | C | A | [symbol] | CDZ98545 |
| X₆₂₅₀ | M6 | 262 | 668764 | C | T | [symbol] | CDZ98545 |
| X₆₂₅₁ | M6 | 262 | 691577 | C | T | [symbol] | CDZ98551 |
| X₆₂₅₂ | M6 | 262 | 722911 | C | A | [symbol] | CDZ98562 |
| X₆₂₅₃ | M6 | 262 | 751209 | C | T | [symbol] | CDZ98572 |
| X₆₂₅₄ | M6 | 262 | 759042 | A | T | [symbol] | CDZ98575 |
| X₆₂₅₅ | M6 | 262 | 822734 | G | A | [symbol] | CDZ98597 |
| X₆₂₅₆ | M6 | 262 | 835874 | C | T | [symbol] | CDZ98601 |
| X₆₂₅₇ | M6 | 262 | 865999 | G | A | [symbol] | CDZ98613 |
| X₆₂₅₈ | M6 | 262 | 950365 | A | T | [symbol] | CDZ98640 |
| X₆₂₅₉ | M6 | 262 | 971713 | G | A | [symbol] | CDZ98649 |
| X₆₂₆₀ | M6 | 262 | 1016286 | C | T | [symbol] | CDZ98661 |
| X₆₂₆₁ | M6 | 262 | 1021485 | C | T | [symbol] | CDZ98663 |
| X₆₂₆₂ | M6 | 262 | 1076581 | G | A | [symbol] | CDZ98679 |
| X₆₂₆₃ | M6 | 262 | 1080137 | G | A | [symbol] | CDZ98679 |
| X₆₂₆₄ | M6 | 262 | 1209662 | G | A | [symbol] | CDZ98723 |
| X₆₂₆₅ | M6 | 262 | 1226072 | C | T | [symbol] | CDZ98730 |
| X₆₂₆₆ | M6 | 262 | 1237021 | C | T | [symbol] | CDZ98735 |
| X₆₂₆₇ | M6 | 262 | 1277604 | G | A | [symbol] | CDZ98747 |
| X₆₂₆₈ | M6 | 262 | 1305361 | G | A | [symbol] | CDZ98753 |
| X₆₂₆₉ | M6 | 262 | 1329643 | C | T | [symbol] | CDZ98760 |
| X₆₂₇₀ | M6 | 262 | 1349561 | G | A | [symbol] | CDZ98766 |
| X₆₂₇₁ | M6 | 262 | 1351116 | A | G | [symbol] | CDZ98766 |
| X₆₂₇₂ | M6 | 262 | 1408928 | G | A | [symbol] | CDZ98786 |
| X₆₂₇₃ | M6 | 262 | 1424293 | G | A | [symbol] | CDZ98791 |
| X₆₂₇₄ | M6 | 262 | 1455677 | G | A | [symbol] | CDZ98804 |
| X₆₂₇₅ | M6 | 262 | 1505836 | G | A | [symbol] | CDZ98823 |
| X₆₂₇₆ | M6 | 262 | 1546246 | C | T | [symbol] | CDZ98833 |
| X₆₂₇₇ | M6 | 262 | 1549845 | C | T | [symbol] | CDZ98837 |
| X₆₂₇₈ | M6 | 262 | 1550311 | C | T | [symbol] | CDZ98836 |
| X₆₂₇₉ | M6 | 262 | 1550456 | C | T | [symbol] | CDZ98837 |
| X₆₂₈₀ | M6 | 262 | 1558074 | G | A | [symbol] | CDZ98840 |
| X₆₂₈₁ | M6 | 262 | 1577316 | G | A | [symbol] | CDZ98844 |
| X₆₂₈₂ | M6 | 262 | 1615762 | G | A | [symbol] | CDZ98855 |
| X₆₂₈₃ | M6 | 262 | 1694625 | C | T | [symbol] | CDZ98878 |
| X₆₂₈₄ | M6 | 262 | 1706076 | G | A | [symbol] | CDZ98885 |
| X₆₂₈₅ | M6 | 262 | 1709112 | G | A | [symbol] | CDZ98884 |
| X₆₂₈₆ | M6 | 262 | 1711527 | G | A | [symbol] | CDZ98886 |
| X₆₂₈₇ | M6 | 262 | 1716385 | G | A | [symbol] | CDZ98887 |
| X₆₂₈₈ | M6 | 265 | 272 | A | G | [symbol] | CDZ98889 |
| X₆₂₈₉ | M6 | 266 | 252 | C | T | [symbol] | |
| X₆₂₉₀ | M6 | 267 | 133 | T | C | [symbol] | |
| X₆₂₉₁ | M6 | 267 | 134 | C | T | [symbol] | |
| X₆₂₉₂ | M7 | 22 | 1643 | C | T | [symbol] | CED80056 |
| X₆₂₉₃ | M7 | 22 | 2524 | G | A | [symbol] | CED80056 |
| X₆₂₉₄ | M7 | 28 | 236 | G | A | [symbol] | CDZ96150 |
| X₆₂₉₅ | M7 | 28 | 377 | G | A | [symbol] | CDZ96150 |
| X₆₂₉₆ | M7 | 28 | 1506 | C | T | [symbol] | CDZ96150 |
| X₆₂₉₇ | M7 | 28 | 1677 | C | T | [symbol] | CDZ96150 |
| X₆₂₉₈ | M7 | 43 | 464 | G | A | [symbol] | CDZ96151 |
| X₆₂₉₉ | M7 | 43 | 753 | C | T | [symbol] | CDZ96151 |
| X₆₃₀₀ | M7 | 43 | 757 | G | A | [symbol] | CDZ96151 |
| X₆₃₀₁ | M7 | 43 | 1527 | C | T | [symbol] | CDZ96151 |
| X₆₃₀₂ | M7 | 43 | 1748 | G | A | [symbol] | CDZ96151 |
| X₆₃₀₃ | M7 | 43 | 1928 | C | T | [symbol] | CDZ96151 |
| X₆₃₀₄ | M7 | 55 | 1405 | C | T | [symbol] | CED80057 |
| X₆₃₀₅ | M7 | 62 | 26 | T | A | [symbol] | CDZ96152 |
| X₆₃₀₆ | M7 | 62 | 27 | T | C | [symbol] | CDZ96152 |
| X₆₃₀₇ | M7 | 62 | 55 | T | A | [symbol] | CDZ96152 |
| X₆₃₀₈ | M7 | 62 | 403 | C | T | [symbol] | CDZ96152 |
| X₆₃₀₉ | M7 | 62 | 669 | C | T | [symbol] | CDZ96152 |
| X₆₃₁₀ | M7 | 62 | 875 | G | A | [symbol] | CDZ96152 |
| X₆₃₁₁ | M7 | 102 | 339 | C | T | [symbol] | CDZ96153 |
| X₆₃₁₂ | M7 | 102 | 951 | G | A | [symbol] | CDZ96153 |
| X₆₃₁₃ | M7 | 102 | 1042 | G | A | [symbol] | CDZ96153 |
| X₆₃₁₄ | M7 | 102 | 2334 | G | A | [symbol] | CDZ96153 |
| X₆₃₁₅ | M7 | 102 | 2590 | C | T | [symbol] | CDZ96153 |
| X₆₃₁₆ | M7 | 102 | 3427 | C | T | [symbol] | CDZ96153 |
| X₆₃₁₇ | M7 | 176 | 358 | G | A | [symbol] | CED80058 |
| X₆₃₁₈ | M7 | 176 | 1872 | G | A | [symbol] | CED80058 |
| X₆₃₁₉ | M7 | 176 | 1980 | G | A | [symbol] | CED80058 |
| X₆₃₂₀ | M7 | 176 | 2335 | A | G | [symbol] | CED80058 |
| X₆₃₂₁ | M7 | 176 | 2349 | G | A | [symbol] | CED80058 |
| X₆₃₂₂ | M7 | 179 | 93 | A | G | [symbol] | CDZ96154 |
| X₆₃₂₃ | M7 | 179 | 96 | G | A | [symbol] | CDZ96154 |
| X₆₃₂₄ | M7 | 179 | 1177 | G | A | [symbol] | CDZ96154 |
| X₆₃₂₅ | M7 | 179 | 1525 | G | A | [symbol] | CDZ96154 |
| X₆₃₂₆ | M7 | 179 | 1739 | G | A | [symbol] | CDZ96154 |
| X₆₃₂₇ | M7 | 179 | 1780 | C | T | [symbol] | CDZ96154 |
| X₆₃₂₈ | M7 | 179 | 2324 | G | A | [symbol] | CDZ96154 |
| X₆₃₂₉ | M7 | 188 | 815 | C | T | [symbol] | CED80060 |
| X₆₃₃₀ | M7 | 188 | 1174 | C | T | [symbol] | CED80060 |
| X₆₃₃₁ | M7 | 188 | 2163 | G | A | [symbol] | CED80060 |
| X₆₃₃₂ | M7 | 188 | 2170 | G | A | [symbol] | CED80060 |
| X₆₃₃₃ | M7 | 247 | 102 | G | A | [symbol] | CED80061 |
| X₆₃₃₄ | M7 | 247 | 989 | C | T | [symbol] | CED80061 |
| X₆₃₃₅ | M7 | 247 | 1053 | G | A | [symbol] | CED80061 |
| X₆₃₃₆ | M7 | 247 | 1454 | C | T | [symbol] | CED80061 |
| X₆₃₃₇ | M7 | 4 | 781 | G | A | [symbol] | |
| X₆₃₃₈ | M7 | 7 | 160 | T | C | [symbol] | |
| X₆₃₃₉ | M7 | 8 | 1110 | C | T | [symbol] | |
| X₆₃₄₀ | M7 | 11 | 154 | A | C | [symbol] | |
| X₆₃₄₁ | M7 | 11 | 158 | C | T | [symbol] | |
| X₆₃₄₂ | M7 | 15 | 28 | C | G | [symbol] | |
| X₆₃₄₃ | M7 | 15 | 462 | C | T | [symbol] | |
| X₆₃₄₄ | M7 | 15 | 464 | C | A | [symbol] | |
| X₆₃₄₅ | M7 | 15 | 465 | C | T | [symbol] | |
| X₆₃₄₆ | M7 | 24 | 513 | T | C | [symbol] | CDZ96160 |
| X₆₃₄₇ | M7 | 24 | 520 | C | G | [symbol] | CDZ96160 |
| X₆₃₄₈ | M7 | 24 | 5850 | C | T | [symbol] | CDZ96160 |
| X₆₃₄₉ | M7 | 24 | 168850 | C | T | [symbol] | CDZ96214 |
| X₆₃₅₀ | M7 | 24 | 172698 | G | A | [symbol] | CDZ96215 |
| X₆₃₅₁ | M7 | 24 | 180340 | G | A | [symbol] | CDZ96216 |
| X₆₃₅₂ | M7 | 24 | 197593 | C | T | [symbol] | CDZ96224 |
| X₆₃₅₃ | M7 | 24 | 266697 | G | A | [symbol] | CDZ96242 |
| X₆₃₅₄ | M7 | 24 | 307114 | G | A | [symbol] | CDZ96255 |
| X₆₃₅₅ | M7 | 24 | 340663 | T | C | [symbol] | CDZ96265 |
| X₆₃₅₆ | M7 | 24 | 349340 | C | T | [symbol] | CDZ96270 |
| X₆₃₅₇ | M7 | 24 | 349352 | G | A | [symbol] | CDZ96270 |
| X₆₃₅₈ | M7 | 24 | 363770 | C | T | [symbol] | CDZ96275 |
| X₆₃₅₉ | M7 | 24 | 382911 | C | T | [symbol] | CDZ96282 |
| X₆₃₆₀ | M7 | 24 | 400418 | C | T | [symbol] | CDZ96287 |
| X₆₃₆₁ | M7 | 24 | 420108 | G | A | [symbol] | CDZ96291 |
| X₆₃₆₂ | M7 | 24 | 470687 | G | A | [symbol] | CDZ96308 |
| X₆₃₆₃ | M7 | 24 | 474698 | G | A | [symbol] | CDZ96308 |
| X₆₃₆₄ | M7 | 24 | 476097 | C | T | [symbol] | CDZ96309 |
| X₆₃₆₅ | M7 | 24 | 488439 | G | T | [symbol] | CDZ96315 |
| X₆₃₆₆ | M7 | 24 | 513701 | G | C | [symbol] | CDZ96326 |
| X₆₃₆₇ | M7 | 27 | 445 | C | T | [symbol] | |
| X₆₃₆₈ | M7 | 27 | 1012 | A | C | [symbol] | |
| X₆₃₆₉ | M7 | 27 | 1013 | T | A | [symbol] | |
| X₆₃₇₀ | M7 | 32 | 158 | C | T | [symbol] | CDZ96332 |
| X₆₃₇₁ | M7 | 32 | 215 | C | T | [symbol] | CDZ96332 |
| X₆₃₇₂ | M7 | 32 | 2438 | C | T | [symbol] | CDZ96332 |
| X₆₃₇₃ | M7 | 32 | 2734 | C | T | [symbol] | CDZ96332 |
| X₆₃₇₄ | M7 | 32 | 2821 | C | T | [symbol] | CDZ96332 |
| X₆₃₇₅ | M7 | 32 | 3067 | C | T | [symbol] | CDZ96332 |
| X₆₃₇₆ | M7 | 32 | 3500 | C | T | [symbol] | CDZ96332 |
| X₆₃₇₇ | M7 | 32 | 3562 | G | A | [symbol] | CDZ96332 |
| X₆₃₇₈ | M7 | 33 | 399 | T | A | [symbol] | CED82002 |
| X₆₃₇₉ | M7 | 33 | 20227 | G | A | [symbol] | CED82006 |
| X₆₃₈₀ | M7 | 33 | 24784 | C | T | [symbol] | CED82008 |
| X₆₃₈₁ | M7 | 33 | 45488 | C | T | [symbol] | CED82012 |
| X₆₃₈₂ | M7 | 33 | 79913 | G | A | [symbol] | CED82024 |
| X₆₃₈₃ | M7 | 33 | 105276 | G | A | [symbol] | CED82036 |
| X₆₃₈₄ | M7 | 33 | 109397 | G | A | [symbol] | CED82038 |
| X₆₃₈₅ | M7 | 33 | 129884 | G | A | [symbol] | CED82045 |
| X₆₃₈₆ | M7 | 33 | 165059 | C | T | [symbol] | CED82056 |
| X₆₃₈₇ | M7 | 33 | 192563 | C | T | [symbol] | CED82066 |
| X₆₃₈₈ | M7 | 33 | 215436 | C | T | [symbol] | CED82071 |
| X₆₃₈₉ | M7 | 33 | 223703 | G | A | [symbol] | CED82075 |
| X₆₃₉₀ | M7 | 33 | 257786 | C | T | [symbol] | CED82084 |
| X₆₃₉₁ | M7 | 33 | 260027 | G | A | [symbol] | CED82084 |
| X₆₃₉₂ | M7 | 33 | 274627 | G | A | [symbol] | CED82091 |
| X₆₃₉₃ | M7 | 33 | 283808 | C | T | [symbol] | CED82093 |
| X₆₃₉₄ | M7 | 33 | 317713 | G | A | [symbol] | CED82104 |
| X₆₃₉₅ | M7 | 33 | 327840 | C | T | [symbol] | CED82108 |
| X₆₃₉₆ | M7 | 33 | 341890 | G | A | [symbol] | CED82113 |
| X₆₃₉₇ | M7 | 33 | 374131 | A | G | [symbol] | CED82121- |
| | | | | | | | CED82122 |
| X₆₃₉₈ | M7 | 33 | 374134 | A | G | [symbol] | CED82121- |
| | | | | | | | CED82122 |
| X₆₃₉₉ | M7 | 33 | 374137 | A | T | [symbol] | CED82121- |
| | | | | | | | CED82122 |
| X₆₄₀₀ | M7 | 33 | 374141 | C | T | [symbol] | CED82121- |
| | | | | | | | CED82122 |
| X₆₄₀₁ | M7 | 33 | 374150 | A | C | [symbol] | CED82121- |
| | | | | | | | CED82122 |
| X₆₄₀₂ | M7 | 33 | 375339 | A | G | [symbol] | CED82121- |
| | | | | | | | CED82122 |
| X₆₄₀₃ | M7 | 33 | 375366 | T | C | [symbol] | CED82121- |
| | | | | | | | CED82122 |
| X₆₄₀₄ | M7 | 33 | 382577 | C | T | [symbol] | CED82122 |
| X₆₄₀₅ | M7 | 33 | 382832 | G | T | [symbol] | CED82122 |
| X₆₄₀₆ | M7 | 33 | 385319 | A | C | [symbol] | CED82122 |
| X₆₄₀₇ | M7 | 33 | 385527 | C | T | [symbol] | CED82122 |
| X₆₄₀₈ | M7 | 33 | 386225 | G | A | [symbol] | CED82124 |
| X₆₄₀₉ | M7 | 33 | 388969 | G | T | [symbol] | CED82124 |
| X₆₄₁₀ | M7 | 33 | 432188 | G | A | [symbol] | CED82137 |
| X₆₄₁₁ | M7 | 33 | 448815 | G | A | [symbol] | CED82144 |
| X₆₄₁₂ | M7 | 33 | 453975 | C | T | [symbol] | CED82147 |
| X₆₄₁₃ | M7 | 33 | 455451 | C | T | [symbol] | CED82146 |
| X₆₄₁₄ | M7 | 33 | 481361 | G | A | [symbol] | CED82157 |
| X₆₄₁₅ | M7 | 33 | 505339 | C | T | [symbol] | CED82165 |
| X₆₄₁₆ | M7 | 33 | 509127 | C | T | [symbol] | CED82166 |
| X₆₄₁₇ | M7 | 33 | 626521 | C | T | [symbol] | CED82203 |
| X₆₄₁₈ | M7 | 33 | 690180 | C | T | [symbol] | CED82224 |
| X₆₄₁₉ | M7 | 33 | 693516 | C | T | [symbol] | CED82225 |
| X₆₄₂₀ | M7 | 33 | 699423 | C | T | [symbol] | CED82227 |
| X₆₄₂₁ | M7 | 33 | 704096 | T | A | [symbol] | CED82227 |
| X₆₄₂₂ | M7 | 33 | 704097 | T | C | [symbol] | CED82227 |
| X₆₄₂₃ | M7 | 33 | 715901 | C | T | [symbol] | CED82231 |
| X₆₄₂₄ | M7 | 33 | 729705 | C | T | [symbol] | CED82234 |
| X₆₄₂₅ | M7 | 33 | 794549 | C | T | [symbol] | CED82254 |
| X₆₄₂₆ | M7 | 33 | 800536 | G | A | [symbol] | CED82259 |
| X₆₄₂₇ | M7 | 33 | 822184 | G | A | [symbol] | CED82264 |
| X₆₄₂₈ | M7 | 33 | 829825 | C | T | [symbol] | CED82271 |
| X₆₄₂₉ | M7 | 33 | 917545 | A | G | [symbol] | CED82294 |
| X₆₄₃₀ | M7 | 33 | 917558 | G | A | [symbol] | CED82294 |
| X₆₄₃₁ | M7 | 33 | 928726 | C | T | [symbol] | CED82295 |
| X₆₄₃₂ | M7 | 33 | 933316 | C | T | [symbol] | CED82299 |
| X₆₄₃₃ | M7 | 33 | 936350 | T | A | [symbol] | CED82300 |
| X₆₄₃₄ | M7 | 33 | 936356 | G | A | [symbol] | CED82300 |
| X₆₄₃₅ | M7 | 33 | 936571 | A | G | [symbol] | CED82300 |
| X₆₄₃₆ | M7 | 33 | 936600 | G | A | [symbol] | CED82300 |
| X₆₄₃₇ | M7 | 33 | 938992 | C | T | [symbol] | CED82300 |
| X₆₄₃₈ | M7 | 33 | 971503 | C | T | [symbol] | CED82307 |
| X₆₄₃₉ | M7 | 33 | 995886 | G | A | [symbol] | CED82318 |
| X₆₄₄₀ | M7 | 33 | 1038506 | C | T | [symbol] | CED82330 |
| X₆₄₄₁ | M7 | 33 | 1049699 | G | A | [symbol] | CED82334 |
| X₆₄₄₂ | M7 | 33 | 1088320 | C | T | [symbol] | CED82349 |
| X₆₄₄₃ | M7 | 33 | 1122362 | C | T | [symbol] | CED82358 |
| X₆₄₄₄ | M7 | 33 | 1126814 | G | A | [symbol] | CED82358 |
| X₆₄₄₅ | M7 | 33 | 1138444 | C | T | [symbol] | CED82363 |
| X₆₄₄₆ | M7 | 33 | 1165914 | G | A | [symbol] | CED82372 |
| X₆₄₄₇ | M7 | 33 | 1191727 | C | T | [symbol] | CED82379 |
| X₆₄₄₈ | M7 | 33 | 1291620 | C | T | [symbol] | CED82406 |
| X₆₄₄₉ | M7 | 33 | 1324395 | G | A | [symbol] | CED82417 |
| X₆₄₅₀ | M7 | 33 | 1339376 | C | T | [symbol] | CED82424 |
| X₆₄₅₁ | M7 | 33 | 1367015 | C | T | [symbol] | CED82432 |
| X₆₄₅₂ | M7 | 33 | 1396438 | C | T | [symbol] | CED82442 |
| X₆₄₅₃ | M7 | 33 | 1460436 | C | T | [symbol] | CED82462 |
| X₆₄₅₄ | M7 | 33 | 1467702 | C | T | [symbol] | CED82463 |
| X₆₄₅₅ | M7 | 33 | 1470375 | C | T | [symbol] | CED82465 |
| X₆₄₅₆ | M7 | 33 | 1499347 | G | A | [symbol] | CED82472 |
| X₆₄₅₇ | M7 | 33 | 1502644 | G | A | [symbol] | CED82474 |
| X₆₄₅₈ | M7 | 33 | 1515369 | G | A | [symbol] | CED82478 |
| X₆₄₅₉ | M7 | 33 | 1535434 | A | T | [symbol] | CED82483 |
| X₆₄₆₀ | M7 | 33 | 1538001 | G | A | [symbol] | CED82483 |
| X₆₄₆₁ | M7 | 33 | 1544767 | A | C | [symbol] | CED82485- |
| | | | | | | | CED82486 |
| X₆₄₆₂ | M7 | 33 | 1544951 | C | G | [symbol] | CED82485- |
| | | | | | | | CED82486 |
| X₆₄₆₃ | M7 | 33 | 1585699 | C | T | [symbol] | CED82497 |
| X₆₄₆₄ | M7 | 33 | 1587443 | C | T | [symbol] | CED82497 |
| X₆₄₆₅ | M7 | 33 | 1596242 | C | T | [symbol] | CED82498 |
| X₆₄₆₆ | M7 | 33 | 1628197 | G | A | [symbol] | CED82510 |
| X₆₄₆₇ | M7 | 33 | 1638726 | G | A | [symbol] | CED82513 |
| X₆₄₆₈ | M7 | 33 | 1642659 | G | A | [symbol] | CED82515 |
| X₆₄₆₉ | M7 | 33 | 1666106 | C | T | [symbol] | CED82522 |
| X₆₄₇₀ | M7 | 33 | 1675912 | C | T | [symbol] | CED82525 |
| X₆₄₇₁ | M7 | 33 | 1700000 | G | A | [symbol] | CED82534 |
| X₆₄₇₂ | M7 | 33 | 1714997 | G | A | [symbol] | CED82538 |
| X₆₄₇₃ | M7 | 33 | 1752414 | G | A | [symbol] | CED82547 |
| X₆₄₇₄ | M7 | 33 | 1767636 | C | T | [symbol] | CED82552 |
| X₆₄₇₅ | M7 | 33 | 1780044 | G | A | [symbol] | CED82555 |
| X₆₄₇₆ | M7 | 33 | 1826223 | G | A | [symbol] | CED82573 |
| X₆₄₇₇ | M7 | 33 | 1841263 | G | A | [symbol] | CED82576 |
| X₆₄₇₈ | M7 | 33 | 1860991 | G | A | [symbol] | CED82583 |
| X₆₄₇₉ | M7 | 33 | 1862200 | G | A | [symbol] | CED82582 |
| X₆₄₈₀ | M7 | 33 | 1887401 | C | T | [symbol] | CED82591 |
| X₆₄₈₁ | M7 | 33 | 1938614 | G | A | [symbol] | CED82612 |
| X₆₄₈₂ | M7 | 33 | 1950090 | G | A | [symbol] | CED82614 |
| X₆₄₈₃ | M7 | 33 | 1967112 | C | T | [symbol] | CED82622 |
| X₆₄₈₄ | M7 | 33 | 1975079 | G | A | [symbol] | CED82625 |
| X₆₄₈₅ | M7 | 33 | 2010230 | C | T | [symbol] | CED82636 |
| X₆₄₈₆ | M7 | 33 | 2014235 | C | T | [symbol] | CED82637 |
| X₆₄₈₇ | M7 | 33 | 2033881 | C | T | [symbol] | CED82645 |
| X₆₄₈₈ | M7 | 33 | 2054662 | G | A | [symbol] | CED82654 |
| X₆₄₈₉ | M7 | 36 | 320 | G | A | [symbol] | |
| X₆₄₉₀ | M7 | 36 | 1056 | G | A | [symbol] | |
| X₆₄₉₁ | M7 | 36 | 1295 | C | T | [symbol] | |
| X₆₄₉₂ | M7 | 36 | 1461 | A | T | [symbol] | |
| X₆₄₉₃ | M7 | 42 | 804 | C | T | [symbol] | CDZ96333 |
| X₆₄₉₄ | M7 | 42 | 1559 | G | A | [symbol] | CDZ96333 |
| X₆₄₉₅ | M7 | 49 | 400 | C | T | [symbol] | |
| X₆₄₉₆ | M7 | 50 | 145 | G | A | [symbol] | |
| X₆₄₉₇ | M7 | 52 | 3042 | A | G | [symbol] | CED82664 |
| X₆₄₉₈ | M7 | 52 | 63859 | C | T | [symbol] | CED82686 |
| X₆₄₉₉ | M7 | 52 | 144382 | C | T | [symbol] | CED82709 |
| X₆₅₀₀ | M7 | 52 | 145060 | G | A | [symbol] | CED82708 |
| X₆₅₀₁ | M7 | 52 | 151974 | C | T | [symbol] | CED82712 |
| X₆₅₀₂ | M7 | 52 | 183986 | C | T | [symbol] | CED82722 |
| X₆₅₀₃ | M7 | 52 | 187553 | C | T | [symbol] | CED82724 |
| X₆₅₀₄ | M7 | 52 | 203253 | A | G | [symbol] | CED82730 |
| X₆₅₀₅ | M7 | 52 | 207354 | C | T | [symbol] | CED82730 |
| X₆₅₀₆ | M7 | 52 | 239820 | A | T | [symbol] | CED82734 |
| X₆₅₀₇ | M7 | 52 | 239821 | A | G | [symbol] | CED82734 |
| X₆₅₀₈ | M7 | 52 | 239825 | T | C | [symbol] | CED82734 |
| X₆₅₀₉ | M7 | 52 | 239827 | A | G | [symbol] | CED82734 |
| X₆₅₁₀ | M7 | 52 | 240075 | G | C | [symbol] | CED82734 |
| X₆₅₁₁ | M7 | 52 | 240241 | T | C | [symbol] | CED82734 |
| X₆₅₁₂ | M7 | 52 | 240245 | A | C | [symbol] | CED82734 |
| X₆₅₁₃ | M7 | 52 | 244305 | G | A | [symbol] | CED82734 |
| X₆₅₁₄ | M7 | 52 | 268499 | G | A | [symbol] | CED82740 |
| X₆₅₁₅ | M7 | 52 | 284733 | G | A | [symbol] | CED82747 |
| X₆₅₁₆ | M7 | 52 | 346628 | G | A | [symbol] | CED82769 |
| X₆₅₁₇ | M7 | 52 | 390095 | G | A | [symbol] | CED82784 |
| X₆₅₁₈ | M7 | 52 | 390988 | C | T | [symbol] | CED82785 |
| X₆₅₁₉ | M7 | 52 | 395374 | G | A | [symbol] | CED82786 |
| X₆₅₂₀ | M7 | 52 | 398723 | C | T | [symbol] | CED82786 |
| X₆₅₂₁ | M7 | 52 | 398728 | C | T | [symbol] | CED82786 |
| X₆₅₂₂ | M7 | 52 | 487472 | C | T | [symbol] | CED82814 |
| X₆₅₂₃ | M7 | 52 | 516267 | C | T | [symbol] | CED82823 |
| X₆₅₂₄ | M7 | 52 | 532992 | G | A | [symbol] | CED82831 |
| X₆₅₂₅ | M7 | 52 | 548788 | G | A | [symbol] | CED82835 |
| X₆₅₂₆ | M7 | 52 | 552099 | G | A | [symbol] | CED82837 |
| X₆₅₂₇ | M7 | 52 | 552173 | G | A | [symbol] | CED82837 |
| X₆₅₂₈ | M7 | 52 | 595806 | C | T | [symbol] | CED82851 |
| X₆₅₂₉ | M7 | 52 | 631479 | C | T | [symbol] | CED82860 |
| X₆₅₃₀ | M7 | 52 | 638622 | G | A | [symbol] | CED82862 |
| X₆₅₃₁ | M7 | 52 | 679670 | G | A | [symbol] | CED82877 |
| X₆₅₃₂ | MZ | 52 | 737419 | G | A | [symbol] | CED82903 |
| X₆₅₃₃ | M7 | 52 | 817452 | T | A | [symbol] | CED82928 |
| X₆₅₃₄ | M7 | 52 | 840755 | C | T | [symbol] | CED82935 |
| X₆₅₃₅ | M7 | 52 | 849671 | G | A | [symbol] | CED82940 |
| X₆₅₃₆ | M7 | 52 | 868578 | C | T | [symbol] | CED82948 |
| X₆₅₃₇ | M7 | 52 | 870083 | G | A | [symbol] | CED82949 |
| X₆₅₃₈ | M7 | 52 | 938650 | G | A | [symbol] | CED82973 |
| X₆₅₃₉ | M7 | 52 | 967316 | A | T | [symbol] | CED82984 |
| X₆₅₄₀ | M7 | 52 | 967701 | T | A | [symbol] | CED82983 |
| X₆₅₄₁ | M7 | 52 | 1003899 | G | A | [symbol] | CED83001 |
| X₆₅₄₂ | M7 | 52 | 1004724 | G | A | [symbol] | CED83001 |
| X₆₅₄₃ | M7 | 52 | 1010006 | C | T | [symbol] | CED83002 |
| X₆₅₄₄ | M7 | 52 | 1029498 | G | A | [symbol] | CED83007 |
| X₆₅₄₅ | M7 | 52 | 1043011 | C | T | [symbol] | CED83012 |
| X₆₅₄₆ | M7 | 52 | 1067895 | C | T | [symbol] | CED83020 |
| X₆₅₄₇ | M7 | 52 | 1151398 | G | A | [symbol] | CED83046 |
| X₆₅₄₈ | M7 | 52 | 1158103 | C | T | [symbol] | CED83051 |
| X₆₅₄₉ | M7 | 52 | 1219894 | C | T | [symbol] | CED83071 |
| X₆₅₅₀ | M7 | 52 | 1227910 | G | A | [symbol] | CED83074 |
| X₆₅₅₁ | M7 | 52 | 1240590 | A | T | [symbol] | CED83078 |
| X₆₅₅₂ | M7 | 52 | 1292343 | G | A | [symbol] | CED83098 |
| X₆₅₅₃ | M7 | 52 | 1328337 | T | A | [symbol] | CED83112 |
| X₆₅₅₄ | M7 | 52 | 1363357 | G | A | [symbol] | CED83122 |
| X₆₅₅₅ | M7 | 52 | 1363405 | C | T | [symbol] | CED83123 |
| X₆₅₅₆ | M7 | 52 | 1373998 | C | T | [symbol] | CED83125 |
| X₆₅₅₇ | M7 | 52 | 1381652 | C | T | [symbol] | CED83129 |
| X₆₅₅₈ | M7 | 52 | 1389320 | G | A | [symbol] | CED83130 |
| X₆₅₅₉ | M7 | 52 | 1392238 | C | T | [symbol] | CED83130 |
| X₆₅₆₀ | M7 | 52 | 1399439 | G | A | [symbol] | CED83134 |
| X₆₅₆₁ | M7 | 52 | 1474315 | G | A | [symbol] | CED83164 |
| X₆₅₆₂ | M7 | 52 | 1475408 | C | T | [symbol] | CED83166 |
| X₆₅₆₃ | M7 | 52 | 1481345 | G | A | [symbol] | CED83165 |
| X₆₅₆₄ | M7 | 52 | 1509004 | G | A | [symbol] | CED83177 |
| X₆₅₆₅ | M7 | 52 | 1520883 | C | T | [symbol] | CED83179 |
| X₆₅₆₆ | M7 | 52 | 1526796 | G | A | [symbol] | CED83181 |
| X₆₅₆₇ | M7 | 52 | 1562633 | C | T | [symbol] | CED83195 |
| X₆₅₆₈ | M7 | 52 | 1583655 | C | T | [symbol] | CED83206 |
| X₆₅₆₉ | M7 | 52 | 1639254 | C | T | [symbol] | CED83227 |
| X₆₅₇₀ | M7 | 52 | 1650115 | C | T | [symbol] | CED83229 |
| X₆₅₇₁ | M7 | 52 | 1662924 | G | A | [symbol] | CED83236 |
| X₆₅₇₂ | M7 | 52 | 1708900 | A | G | [symbol] | CED83249 |
| X₆₅₇₃ | M7 | 52 | 1716210 | C | T | [symbol] | CED83250 |
| X₆₅₇₄ | M7 | 52 | 1793370 | G | A | [symbol] | CED83275 |
| X₆₅₇₅ | M7 | 52 | 1807309 | G | A | [symbol] | CED83283 |
| X₆₅₇₆ | M7 | 52 | 1826635 | G | A | [symbol] | CED83289 |
| X₆₅₇₇ | M7 | 52 | 1864608 | G | A | [symbol] | CED83303 |
| X₆₅₇₈ | M7 | 52 | 1871310 | C | T | [symbol] | CED83304 |
| X₆₅₇₉ | M7 | 52 | 1874150 | G | T | [symbol] | CED83305 |
| X₆₅₈₀ | M7 | 52 | 1874531 | C | T | [symbol] | CED83306 |
| X₆₅₈₁ | M7 | 52 | 1888989 | C | T | [symbol] | CED83312 |
| X₆₅₈₂ | M7 | 52 | 1893352 | G | A | [symbol] | CED83313 |
| X₆₅₈₃ | M7 | 52 | 1915927 | G | A | [symbol] | CED83315 |
| X₆₅₈₄ | M7 | 52 | 1938393 | C | T | [symbol] | CED83322 |
| X₆₅₈₅ | M7 | 52 | 1968215 | A | G | [symbol] | CED83335 |
| X₆₅₈₆ | M7 | 52 | 1969873 | C | T | [symbol] | CED83333 |
| X₆₅₈₇ | M7 | 52 | 1987096 | G | A | [symbol] | CED83341 |
| X₆₅₈₈ | M7 | 52 | 1995167 | G | A | [symbol] | CED83342 |
| X₆₅₈₉ | M7 | 52 | 2041781 | C | T | [symbol] | CED83355 |
| X₆₅₉₀ | M7 | 52 | 2050245 | G | A | [symbol] | CED83359 |
| X₆₅₉₁ | M7 | 52 | 2081390 | G | A | [symbol] | CED83367 |
| X₆₅₉₂ | M7 | 52 | 2093148 | C | T | [symbol] | CED83368 |
| X₆₅₉₃ | M7 | 52 | 2120665 | C | T | [symbol] | CED83376 |
| X₆₅₉₄ | M7 | 52 | 2136885 | A | G | [symbol] | CED83382 |
| X₆₅₉₅ | M7 | 52 | 2162878 | G | A | [symbol] | CED83392 |
| X₆₅₉₆ | M7 | 52 | 2171271 | C | T | [symbol] | CED83395 |
| X₆₅₉₇ | M7 | 52 | 2258192 | C | T | [symbol] | CED83422 |
| X₆₅₉₈ | M7 | 52 | 2265219 | C | T | [symbol] | CED83425 |
| X₆₅₉₉ | M7 | 52 | 2266378 | C | T | [symbol] | CED83423 |
| X₆₆₀₀ | M7 | 52 | 2308421 | T | C | [symbol] | CED83439 |
| X₆₆₀₁ | M7 | 52 | 2327266 | C | T | [symbol] | CED83445 |
| X₆₆₀₂ | M7 | 52 | 2338728 | G | A | [symbol] | CED83451 |
| X₆₆₀₃ | M7 | 52 | 2367582 | G | A | [symbol] | CED83455 |
| X₆₆₀₄ | M7 | 52 | 2373120 | C | T | [symbol] | CED83456 |
| X₆₆₀₅ | M7 | 52 | 2373426 | G | A | [symbol] | CED83456 |
| X₆₆₀₆ | M7 | 52 | 2373448 | C | T | [symbol] | CED83456 |
| X₆₆₀₇ | M7 | 52 | 2400778 | C | T | [symbol] | CED83463 |
| X₆₆₀₈ | M7 | 52 | 2531584 | C | T | [symbol] | 1CED83507 |
| X₆₆₀₉ | M7 | 52 | 2543220 | C | T | [symbol] | CED83509 |
| X₆₆₁₀ | M7 | 52 | 2574904 | G | T | [symbol] | CED83525 |
| X₆₆₁₁ | M7 | 52 | 2575090 | A | G | [symbol] | CED83525 |
| X₆₆₁₂ | M7 | 53 | 26291 | G | A | [symbol] | CDZ96348 |
| X₆₆₁₃ | M7 | 53 | 69664 | C | T | [symbol] | CDZ96363 |
| X₆₆₁₄ | M7 | 53 | 89575 | C | T | [symbol] | CDZ96369 |
| X₆₆₁₅ | M7 | 53 | 104248 | G | A | [symbol] | CDZ96376 |
| X₆₆₁₆ | M7 | 53 | 110192 | C | T | [symbol] | CDZ96376 |
| X₆₆₁₇ | M7 | 54 | 4815 | G | A | [symbol] | CDZ96384 |
| X₆₆₁₈ | M7 | 54 | 4816 | A | T | [symbol] | CDZ96384 |
| X₆₆₁₉ | M7 | 54 | 4817 | G | C | [symbol] | CDZ96384 |
| X₆₆₂₀ | M7 | 54 | 4821 | G | A | [symbol] | CDZ96384 |
| X₆₆₂₁ | M7 | 54 | 16963 | C | T | [symbol] | CDZ96386 |
| X₆₆₂₂ | M7 | 54 | 30229 | T | C | [symbol] | CDZ96390 |
| X₆₆₂₃ | M7 | 54 | 31207 | G | A | [symbol] | CDZ96392 |
| X₆₆₂₄ | M7 | 54 | 87947 | G | A | [symbol] | CDZ96410 |
| X₆₆₂₅ | M7 | 54 | 95921 | C | T | [symbol] | CDZ96413 |
| X₆₆₂₆ | M7 | 54 | 106086 | G | A | [symbol] | CDZ96416 |
| X₆₆₂₇ | M7 | 54 | 119439 | C | T | [symbol] | CDZ96420 |
| X₆₆₂₈ | M7 | 54 | 119791 | C | T | [symbol] | CDZ96419 |
| X₆₆₂₉ | M7 | 54 | 167759 | A | T | [symbol] | CDZ96438 |
| X₆₆₃₀ | M7 | 54 | 194230 | G | A | [symbol] | CDZ96446 |
| X₆₆₃₁ | M7 | 54 | 195804 | G | A | [symbol] | CDZ96447 |
| X₆₆₃₂ | M7 | 54 | 202467 | C | T | [symbol] | CDZ96448 |
| X₆₆₃₃ | M7 | 54 | 206494 | G | A | [symbol] | CDZ96450 |
| X₆₆₃₄ | M7 | 54 | 207267 | G | A | [symbol] | CDZ96450 |
| X₆₆₃₅ | M7 | 54 | 274617 | A | T | [symbol] | CDZ96471 |
| X₆₆₃₆ | M7 | 54 | 278479 | C | T | [symbol] | CDZ96474 |
| X₆₆₃₇ | M7 | 54 | 316790 | T | G | [symbol] | CDZ96487 |
| X₆₆₃₈ | M7 | 54 | 337228 | G | A | [symbol] | CDZ96488 |
| X₆₆₃₉ | M7 | 54 | 357149 | G | A | [symbol] | CDZ96496 |
| X₆₆₄₀ | M7 | 54 | 370320 | G | A | [symbol] | CDZ96499 |
| X₆₆₄₁ | M7 | 54 | 383172 | C | T | [symbol] | CDZ96506 |
| X₆₆₄₂ | M7 | 54 | 391223 | A | G | [symbol] | CDZ96509 |
| X₆₆₄₃ | M7 | 54 | 404968 | C | T | [symbol] | CDZ96513 |
| X₆₆₄₄ | M7 | 54 | 427542 | C | T | [symbol] | CDZ96520 |
| X₆₆₄₅ | M7 | 54 | 431404 | C | T | [symbol] | CDZ96520 |
| X₆₆₄₆ | M7 | 54 | 431851 | C | T | [symbol] | CDZ96520 |
| X₆₆₄₇ | M7 | 54 | 477453 | C | T | [symbol] | CDZ96537 |
| X₆₆₄₈ | M7 | 54 | 478527 | C | T | [symbol] | CDZ96537 |
| X₆₆₄₉ | M7 | 54 | 509857 | G | A | [symbol] | CDZ96544 |
| X₆₆₅₀ | M7 | 54 | 515478 | A | G | [symbol] | CDZ96546 |
| X₆₆₅₁ | M7 | 54 | 555986 | G | A | [symbol] | CDZ96563 |
| X₆₆₅₂ | M7 | 54 | 616268 | A | G | [symbol] | CDZ96581 |
| X₆₆₅₃ | M7 | 54 | 648472 | G | A | [symbol] | CDZ96590 |
| X₆₆₅₄ | M7 | 54 | 686223 | C | T | [symbol] | CDZ96602 |
| X₆₆₅₅ | M7 | 54 | 699570 | G | A | [symbol] | CDZ96609 |
| X₆₆₅₆ | M7 | 54 | 733193 | T | C | [symbol] | CDZ96619 |
| X₆₆₅₇ | M7 | 54 | 743396 | G | A | [symbol] | CDZ96621 |
| X₆₆₅₈ | M7 | 58 | 93 | T | C | [symbol] | |
| X₆₆₅₉ | M7 | 64 | 5037 | G | A | [symbol] | CDZ96626 |
| X₆₆₆₀ | M7 | 67 | 111 | G | A | [symbol] | |
| X₆₆₆₁ | M7 | 69 | 29006 | G | A | [symbol] | CED83534 |
| X₆₆₆₂ | M7 | 69 | 50642 | C | T | [symbol] | CED83543 |
| X₆₆₆₃ | M7 | 69 | 55826 | G | A | [symbol] | CED83545 |
| X₆₆₆₄ | M7 | 69 | 82710 | G | A | [symbol] | CED83554 |
| X₆₆₆₅ | M7 | 69 | 84820 | G | A | [symbol] | CED83555 |
| X₆₆₆₆ | M7 | 69 | 107920 | C | T | [symbol] | CED83561 |
| X₆₆₆₇ | M7 | 69 | 123084 | C | T | [symbol] | CED83563 |
| X₆₆₆₈ | M7 | 69 | 148757 | C | T | [symbol] | CED83575 |
| X₆₆₆₉ | M7 | 69 | 201005 | G | A | [symbol] | CED83595 |
| X₆₆₇₀ | M7 | 69 | 228938 | C | T | [symbol] | CED83603 |
| X₆₆₇₁ | M7 | 69 | 305997 | C | T | [symbol] | CED83631 |
| X₆₆₇₂ | M7 | 69 | 314003 | C | T | [symbol] | CED83634 |
| X₆₆₇₃ | M7 | 69 | 384418 | G | A | [symbol] | CED83656 |
| X₆₆₇₄ | M7 | 69 | 386371 | C | T | [symbol] | CED83657 |
| X₆₆₇₅ | M7 | 69 | 415659 | C | T | [symbol] | CED83665 |
| X₆₆₇₆ | M7 | 69 | 455639 | G | A | [symbol] | CED83677 |
| X₆₆₇₇ | M7 | 69 | 461021 | C | T | [symbol] | CED83680 |
| X₆₆₇₈ | M7 | 69 | 461986 | G | A | [symbol] | CED83680 |
| X₆₆₇₉ | M7 | 69 | 467686 | C | T | [symbol] | CED83681 |
| X₆₆₈₀ | M7 | 69 | 473113 | G | A | [symbol] | CED83684 |
| X₆₆₈₁ | M7 | 69 | 534495 | C | T | [symbol] | CED83704 |
| X₆₆₈₂ | M7 | 69 | 560348 | G | A | [symbol] | CED83713 |
| X₆₆₈₈ | M7 | 69 | 585845 | G | A | [symbol] | CED83722 |
| X₆₆₈₄ | M7 | 69 | 627928 | C | T | [symbol] | CED83740 |
| X₆₆₈₅ | M7 | 69 | 631511 | G | A | [symbol] | CED83742 |
| X₆₆₈₆ | M7 | 69 | 637299 | T | C | [symbol] | CED83744 |
| X₆₆₈₇ | M7 | 69 | 675579 | G | A | [symbol] | CED83760 |
| X₆₆₈₈ | M7 | 69 | 676581 | G | A | [symbol] | CED83760 |
| X₆₆₈₉ | M7 | 69 | 676853 | G | A | [symbol] | CED83759 |
| X₆₆₉₀ | M7 | 69 | 678406 | C | T | [symbol] | CED83759 |
| X₆₆₉₁ | M7 | 69 | 707651 | G | A | [symbol] | CED83771 |
| X₆₆₉₂ | M7 | 69 | 743019 | C | T | [symbol] | CED83782 |
| X₆₆₉₃ | M7 | 69 | 744021 | C | T | [symbol] | CED83780 |
| X₆₆₉₄ | M7 | 69 | 828676 | C | T | [symbol] | CED83805 |
| X₆₆₉₅ | M7 | 69 | 840268 | C | T | [symbol] | CED83808 |
| X₆₆₉₆ | M7 | 69 | 843668 | G | A | [symbol] | CED83809 |
| X₆₆₉₇ | M7 | 69 | 853384 | C | T | [symbol] | CED83809 |
| X₆₆₉₈ | M7 | 69 | 865820 | C | T | [symbol] | CED83814 |
| X₆₆₉₉ | M7 | 69 | 869706 | G | A | [symbol] | CED83815 |
| X₆₇₀₀ | M7 | 69 | 892199 | G | A | [symbol] | CED83822 |
| X₆₇₀₁ | M7 | 69 | 894462 | C | T | [symbol] | CED83821 |
| X₆₇₀₂ | M7 | 69 | 930700 | C | T | [symbol] | CED83835 |
| X₆₇₀₃ | M7 | 69 | 931399 | T | A | [symbol] | CED83835 |
| X₆₇₀₄ | M7 | 69 | 971219 | C | T | [symbol] | CED83847 |
| X₆₇₀₅ | M7 | 69 | 988364 | G | A | [symbol] | CED83857 |
| X₆₇₀₆ | M7 | 69 | 989781 | G | A | [symbol] | CED83858 |
| X₆₇₀₇ | M7 | 69 | 994927 | C | T | [symbol] | CED83859 |
| X₆₇₀₈ | M7 | 69 | 1017805 | G | A | [symbol] | CED83865 |
| X₆₇₀₉ | M7 | 69 | 1021695 | G | A | [symbol] | CED83868 |
| X₆₇₁₀ | M7 | 69 | 1042212 | G | A | [symbol] | CED83875 |
| X₆₇₁₁ | M7 | 69 | 1072756 | G | A | [symbol] | CED83886 |
| X₆₇₁₂ | M7 | 69 | 1080578 | G | A | [symbol] | CED83890 |
| X₆₇₁₃ | M7 | 69 | 1084824 | G | A | [symbol] | CED83891 |
| X₆₇₁₄ | M7 | 69 | 1084835 | C | T | [symbol] | CED83891 |
| X₆₇₁₅ | M7 | 69 | 1084848 | A | G | [symbol] | CED83891 |
| X₆₇₁₆ | M7 | 69 | 1084866 | A | G | [symbol] | CED83891 |
| X₆₇₁₇ | M7 | 69 | 1084931 | C | T | [symbol] | CED83891 |
| X₆₇₁₈ | M7 | 69 | 1102164 | C | T | [symbol] | CED83897 |
| X₆₇₁₉ | M7 | 69 | 1109785 | C | T | [symbol] | CED83899 |
| X₆₇₂₀ | M7 | 69 | 1109813 | A | G | [symbol] | CED83899 |
| X₆₇₂₁ | M7 | 69 | 1109818 | A | G | [symbol] | CED83899 |
| X₆₇₂₂ | M7 | 69 | 1109819 | T | A | [symbol] | CED83899 |
| X₆₇₂₃ | M7 | 69 | 1109823 | T | C | [symbol] | CED83899 |
| X₆₇₂₄ | M7 | 69 | 1109830 | A | G | [symbol] | CED83899 |
| X₆₇₂₅ | M7 | 69 | 1109831 | C | A | [symbol] | CED83899 |
| X₆₇₂₆ | M7 | 69 | 1109835 | A | G | [symbol] | CED83899 |
| X₆₇₂₇ | M7 | 69 | 1110547 | A | G | [symbol] | CED83899 |
| X₆₇₂₈ | M7 | 69 | 1110548 | T | C | [symbol] | CED83899 |
| X₆₇₂₉ | M7 | 69 | 1115486 | C | T | [symbol] | CED83902 |
| X₆₇₃₀ | M7 | 69 | 1152012 | G | A | [symbol] | CED83915 |
| X₆₇₃₁ | M7 | 69 | 1199155 | C | T | [symbol] | CED83929 |
| X₆₇₃₂ | M7 | 69 | 1205454 | C | T | [symbol] | CED83932 |
| X₆₇₃₃ | M7 | 69 | 1212727 | G | A | [symbol] | CED83933 |
| X₆₇₃₄ | M7 | 69 | 1218409 | G | A | [symbol] | CED83936 |
| X₆₇₃₅ | M7 | 69 | 1237631 | T | C | [symbol] | CED83942 |
| X₆₇₃₆ | M7 | 69 | 1260087 | G | A | [symbol] | CED83949 |
| X₆₇₃₇ | M7 | 69 | 1279557 | G | A | [symbol] | CED83959 |
| X₆₇₃₈ | M7 | 69 | 1285126 | A | G | [symbol] | CED83961 |
| X₆₇₃₉ | M7 | 69 | 1342909 | G | A | [symbol] | CED83975 |
| X₆₇₄₀ | M7 | 69 | 1372486 | C | T | [symbol] | CED83987 |
| X₆₇₄₁ | M7 | 69 | 1400546 | G | A | [symbol] | CED83995 |
| X₆₇₄₂ | M7 | 69 | 1411237 | C | T | [symbol] | CED84001 |
| X₆₇₄₃ | M7 | 69 | 1411421 | C | T | [symbol] | CED83999 |
| X₆₇₄₄ | M7 | 69 | 1423154 | G | A | [symbol] | CED84004 |
| X₆₇₄₅ | M7 | 69 | 1519151 | C | T | [symbol] | CED84034 |
| X₆₇₄₆ | M7 | 69 | 1521875 | C | T | [symbol] | CED84035 |
| X₆₇₄₇ | M7 | 69 | 1526612 | T | C | [symbol] | CED84036 |
| X₆₇₄₈ | M7 | 69 | 1530586 | C | T | [symbol] | CED84038 |
| X₆₇₄₉ | M7 | 69 | 1590302 | G | A | [symbol] | CED84059 |
| X₆₇₅₀ | M7 | 69 | 1642484 | C | T | [symbol] | CED84076 |
| X₆₇₅₁ | M7 | 69 | 1652288 | C | T | [symbol] | CED84077 |
| X₆₇₅₂ | M7 | 69 | 1674261 | C | T | [symbol] | CED84084 |
| X₆₇₅₃ | M7 | 69 | 1688555 | C | T | [symbol] | CED84088 |
| X₆₇₅₄ | M7 | 69 | 1714728 | G | A | [symbol] | CED84097 |
| X₆₇₅₅ | M7 | 69 | 1720116 | G | A | [symbol] | CED84102 |
| X₆₇₅₆ | M7 | 69 | 1771849 | C | T | [symbol] | CED84116 |
| X₆₇₅₇ | M7 | 69 | 1788929 | G | A | [symbol] | CED84124 |
| X₆₇₅₈ | M7 | 69 | 1790823 | C | T | [symbol] | CED84125 |
| X₆₇₅₉ | M7 | 69 | 1792597 | C | T | [symbol] | CED84126 |
| X₆₇₆₀ | M7 | 69 | 1800584 | G | A | [symbol] | CED84129 |
| X₆₇₆₁ | M7 | 69 | 1854739 | G | A | [symbol] | CED84145 |
| X₆₇₆₂ | M7 | 69 | 1878663 | C | T | [symbol] | CED84154 |
| X₆₇₆₃ | M7 | 69 | 1924621 | C | T | [symbol] | CED84172 |
| X₆₇₆₄ | M7 | 69 | 1957485 | C | T | [symbol] | CED84187 |
| X₆₇₆₅ | M7 | 69 | 1967529 | C | T | [symbol] | CED84189 |
| X₆₇₆₆ | M7 | 69 | 1968848 | A | G | [symbol] | CED84189 |
| X₆₇₆₇ | M7 | 69 | 1975946 | G | A | [symbol] | CED84193 |
| X₆₇₆₈ | M7 | 69 | 2001469 | C | T | [symbol] | CED84197 |
| X₆₇₆₉ | M7 | 69 | 2048332 | C | T | [symbol] | CED84217 |
| X₆₇₇₀ | M7 | 69 | 2075336 | C | T | [symbol] | CED84226 |
| X₆₇₇₁ | M7 | 69 | 2099418 | C | T | [symbol] | CED84233 |
| X₆₇₇₂ | M7 | 69 | 2113590 | G | A | [symbol] | CED84237 |
| X₆₇₇₃ | M7 | 69 | 2127656 | T | A | [symbol] | CED84242 |
| X₆₇₇₄ | M7 | 69 | 2160207 | C | T | [symbol] | CED84255 |
| X₆₇₇₅ | M7 | 69 | 2180564 | C | T | [symbol] | CED84264 |
| X₆₇₇₆ | M7 | 69 | 2226317 | C | T | [symbol] | CED84272 |
| X₆₇₇₇ | M7 | 69 | 2231926 | G | A | [symbol] | CED84274 |
| X₆₇₇₈ | M7 | 69 | 2240834 | C | T | [symbol] | CED84277 |
| X₆₇₇₉ | M7 | 69 | 2255001 | G | A | [symbol] | CED84282 |
| X₆₇₈₀ | M7 | 69 | 2283903 | G | A | [symbol] | CED84293 |
| X₆₇₈₁ | M7 | 69 | 2325544 | C | T | [symbol] | CED84307 |
| X₆₇₈₂ | M7 | 69 | 2438727 | C | T | [symbol] | CED84343 |
| X₆₇₈₃ | M7 | 69 | 2448630 | C | T | [symbol] | CED84344 |
| X₆₇₈₄ | M7 | 69 | 2481008 | G | A | [symbol] | CED84352 |
| X₆₇₈₅ | M7 | 69 | 2483433 | G | A | [symbol] | CED84352 |
| X₆₇₈₆ | M7 | 72 | 93 | T | C | [symbol] | |
| X₆₇₈₇ | M7 | 72 | 584 | G | A | [symbol] | |
| X₆₇₈₈ | M7 | 72 | 856 | C | T | [symbol] | |
| X₆₇₈₉ | M7 | 72 | 1163 | C | T | [symbol] | |
| X₆₇₉₀ | M7 | 77 | 18912 | A | G | [symbol] | CDZ96635 |
| X₆₇₉₁ | M7 | 77 | 26888 | G | A | [symbol] | CDZ96641 |
| X₆₇₉₂ | M7 | 77 | 34978 | G | T | [symbol] | CDZ96642 |
| X₆₇₉₃ | M7 | 77 | 39842 | G | A | [symbol] | CDZ96646 |
| X₆₇₉₄ | M7 | 77 | 84022 | G | A | [symbol] | CDZ96659 |
| X₆₇₉₅ | M7 | 77 | 105644 | G | A | [symbol] | CDZ96669 |
| X₆₇₉₆ | M7 | 78 | 6333 | G | A | [symbol] | CED84360 |
| X₆₇₉₇ | M7 | 78 | 26915 | C | T | [symbol] | CED84366 |
| X₆₇₉₈ | M7 | 78 | 35200 | G | A | [symbol] | CED84371 |
| X₆₇₉₉ | M7 | 78 | 66886 | G | A | [symbol] | CED84381 |
| X₆₈₀₀ | M7 | 78 | 158534 | G | A | [symbol] | CED84406 |
| X₆₈₀₁ | M7 | 78 | 238854 | C | T | [symbol] | CED84430 |
| X₆₈₀₂ | M7 | 78 | 263896 | T | C | [symbol] | CED84438 |
| X₆₈₀₃ | M7 | 78 | 281454 | C | A | [symbol] | CED84441 |
| X₆₈₀₄ | M7 | 78 | 292068 | C | T | [symbol] | CED84442 |
| X₆₈₀₅ | M7 | 78 | 298141 | C | G | [symbol] | CED84443 |
| X₆₈₀₆ | M7 | 78 | 299166 | G | A | [symbol] | CED84443 |
| X₆₈₀₇ | M7 | 78 | 299644 | G | A | [symbol] | CED84443 |
| X₆₈₀₈ | M7 | 78 | 302700 | A | G | [symbol] | CED84443 |
| X₆₈₀₉ | M7 | 78 | 302816 | T | C | [symbol] | CED84443 |
| X₆₈₁₀ | M7 | 78 | 304799 | A | C | [symbol] | CED84444 |
| X₆₈₁₁ | M7 | 78 | 333312 | A | G | [symbol] | CED84452 |
| X₆₈₁₂ | M7 | 78 | 386356 | C | T | [symbol] | CED84472 |
| X₆₈₁₃ | M7 | 78 | 444292 | C | T | [symbol] | CED84490 |
| X₆₈₁₄ | M7 | 78 | 480244 | C | T | [symbol] | CED84502 |
| X₆₈₁₅ | M7 | 78 | 492399 | C | T | [symbol] | CED84504 |
| X₆₈₁₆ | M7 | 78 | 626958 | C | A | [symbol] | CED84549 |
| X₆₈₁₇ | M7 | 78 | 658932 | G | A | [symbol] | CED84561 |
| X₆₈₁₈ | M7 | 78 | 666005 | C | T | [symbol] | CED84562 |
| X₆₈₁₉ | M7 | 78 | 710966 | G | A | [symbol] | CED84581 |
| X₆₈₂₀ | M7 | 78 | 715700 | G | A | [symbol] | CED84582 |
| X₆₈₂₁ | M7 | 78 | 718885 | C | T | [symbol] | CED84584 |
| X₆₈₂₂ | M7 | 78 | 763771 | G | A | [symbol] | CED84600 |
| X₆₈₂₃ | M7 | 78 | 774252 | G | A | [symbol] | CED84602 |
| X₆₈₂₄ | M7 | 78 | 853752 | G | A | [symbol] | CED84625 |
| X₆₈₂₅ | M7 | 78 | 895505 | C | T | [symbol] | CED84642 |
| X₆₈₂₆ | M7 | 78 | 928773 | C | T | [symbol] | CED84653 |
| X₆₈₂₇ | M7 | 78 | 939705 | G | A | [symbol] | CED84658 |
| X₆₈₂₈ | M7 | 78 | 952815 | G | A | [symbol] | CED84662 |
| X₆₈₂₉ | M7 | 78 | 952860 | G | A | [symbol] | CED84662 |
| X₆₈₃₀ | M7 | 78 | 976680 | C | T | [symbol] | CED84672 |
| X₆₈₃₁ | M7 | 78 | 976799 | C | T | [symbol] | CED84672 |
| X₆₈₃₂ | M7 | 78 | 1023381 | G | A | [symbol] | CED84688 |
| X₆₈₃₃ | M7 | 78 | 1029603 | A | T | [symbol] | CED84689 |
| X₆₈₃₄ | M7 | 78 | 1039555 | C | T | [symbol] | CED84695 |
| X₆₈₃₅ | M7 | 78 | 1077392 | T | C | [symbol] | CED84708 |
| X₆₈₃₆ | M7 | 78 | 1086537 | C | T | [symbol] | CED84713 |
| X₆₈₃₇ | M7 | 78 | 1111002 | G | A | [symbol] | CED84721 |
| X₆₈₃₈ | M7 | 78 | 1115511 | G | A | [symbol] | CED84723 |
| X₆₈₃₉ | M7 | 78 | 1199558 | G | A | [symbol] | CED84746 |
| X₆₈₄₀ | M7 | 78 | 1216573 | C | T | [symbol] | CED84752 |
| X₆₈₄₁ | M7 | 78 | 1289816 | C | T | [symbol] | CED84778 |
| X₆₈₄₂ | M7 | 78 | 1302286 | C | T | [symbol] | CED84782 |
| X₆₈₄₃ | M7 | 78 | 1315648 | C | T | [symbol] | CED84788 |
| X₆₈₄₄ | M7 | 78 | 1317124 | G | A | [symbol] | CED84787 |
| X₆₈₄₅ | M7 | 78 | 1320778 | G | A | [symbol] | CED84790 |
| X₆₈₄₆ | M7 | 78 | 1344640 | C | T | [symbol] | CED84795 |
| X₆₈₄₇ | M7 | 78 | 1426722 | C | T | [symbol] | CED84829 |
| X₆₈₄₈ | M7 | 78 | 1493542 | C | T | [symbol] | CED84851 |
| X₆₈₄₉ | M7 | 78 | 1532973 | G | A | [symbol] | CED84857 |
| X₆₈₅₀ | M7 | 78 | 1549659 | G | A | [symbol] | CED84862 |
| X₆₈₅₁ | M7 | 78 | 1568326 | C | T | [symbol] | CED84868 |
| X₆₈₅₂ | M7 | 78 | 1642170 | G | A | [symbol] | CED84889 |
| X₆₈₅₃ | M7 | 78 | 1658512 | G | A | [symbol] | CED84896 |
| X₆₈₅₄ | M7 | 78 | 1660408 | G | A | [symbol] | CED84897 |
| X₆₈₅₅ | M7 | 78 | 1674126 | G | A | [symbol] | CED84904 |
| X₆₈₅₆ | M7 | 78 | 1694684 | C | T | [symbol] | CED84912 |
| X₆₈₅₇ | M7 | 78 | 1721661 | G | A | [symbol] | CED84921 |
| X₆₀₅₈ | M7 | 78 | 1742796 | G | A | [symbol] | CED84926 |
| X₆₈₅₉ | M7 | 79 | 65 | G | A | [symbol] | CDZ96674 |
| X₆₈₆₀ | M7 | 79 | 133 | A | G | [symbol] | CDZ96674 |
| X₆₈₆₁ | M7 | 79 | 41271 | C | T | [symbol] | CDZ96684 |
| X₆₈₆₂ | M7 | 79 | 57804 | G | A | [symbol] | CDZ96687 |
| X₆₈₆₃ | M7 | 79 | 64658 | C | T | [symbol] | CDZ96688 |
| X₆₈₆₄ | M7 | 79 | 132683 | C | T | [symbol] | CDZ96713 |
| X₆₈₆₅ | M7 | 79 | 168708 | C | T | [symbol] | CDZ96727 |
| X₆₈₆₆ | M7 | 79 | 241350 | C | T | [symbol] | CDZ96753 |
| X₆₈₆₇ | M7 | 79 | 255780 | G | A | [symbol] | CDZ96757 |
| X₆₈₆₈ | M7 | 79 | 273231 | G | A | [symbol] | CDZ96764 |
| X₆₈₆₉ | M7 | 79 | 296092 | G | A | [symbol] | CDZ96772 |
| X₆₈₇₀ | M7 | 79 | 307021 | C | T | [symbol] | CDZ96777 |
| X₆₈₇₁ | M7 | 79 | 324145 | C | T | [symbol] | CDZ96781 |
| X₆₈₇₂ | M7 | 79 | 332458 | G | A | [symbol] | CDZ96785 |
| X₆₈₇₃ | M7 | 79 | 345646 | G | A | [symbol] | CDZ96789 |
| X₆₈₇₄ | M7 | 79 | 346823 | G | A | [symbol] | CDZ96789 |
| X₆₈₇₅ | M7 | 79 | 369258 | G | A | [symbol] | CDZ96793 |
| X₆₈₇₆ | M7 | 79 | 406184 | G | A | [symbol] | CDZ96808 |
| X₆₈₇₇ | M7 | 79 | 451849 | G | A | [symbol] | CDZ96827 |
| X₆₈₇₈ | M7 | 79 | 478890 | C | T | [symbol] | CDZ96834 |
| X₆₈₇₉ | M7 | 79 | 492523 | C | T | [symbol] | CDZ96842 |
| X₆₈₈₀ | M7 | 79 | 570874 | C | T | [symbol] | CDZ96867 |
| X₆₈₈₁ | M7 | 79 | 575411 | C | T | [symbol] | CDZ96868 |
| X₆₈₈₂ | M7 | 79 | 579674 | G | A | [symbol] | CDZ96871 |
| X₆₈₈₃ | M7 | 79 | 617057 | C | T | [symbol] | CDZ96882 |
| X₆₈₈₄ | M7 | 79 | 679140 | C | T | [symbol] | CDZ96904 |
| X₆₈₈₅ | M7 | 79 | 696174 | G | A | [symbol] | CDZ96911 |
| X₆₈₈₆ | M7 | 79 | 735053 | T | C | [symbol] | CDZ96928 |
| X₆₈₈₇ | M7 | 79 | 747717 | A | G | [symbol] | CDZ96929 |
| X₆₈₈₈ | M7 | 79 | 748083 | G | T | [symbol] | CDZ96929 |
| X₆₈₈₉ | M7 | 79 | 749595 | A | C | [symbol] | CDZ96930 |
| X₆₈₉₀ | M7 | 79 | 759195 | T | A | [symbol] | CDZ96931 |
| X₆₈₉₁ | M7 | 79 | 759403 | C | T | [symbol] | CDZ96931 |
| X₆₈₉₂ | M7 | 79 | 759589 | G | A | [symbol] | CDZ96932 |
| X₆₈₉₃ | M7 | 79 | 759659 | G | A | [symbol] | CDZ96931 |
| X₆₈₉₄ | M7 | 79 | 760458 | G | T | [symbol] | CDZ96932 |
| X₆₈₉₅ | M7 | 79 | 760891 | A | G | [symbol] | CDZ96932 |
| X₆₈₉₆ | M7 | 79 | 761056 | T | C | [symbol] | CDZ96932 |
| X₆₈₉₇ | M7 | 79 | 768537 | A | G | [symbol] | CDZ96933 |
| X₆₈₉₈ | M7 | 79 | 776112 | A | G | [symbol] | CDZ96934 |
| X₆₈₉₉ | M7 | 79 | 776150 | G | A | [symbol] | CDZ96934 |
| X₆₉₀₀ | M7 | 79 | 798799 | C | T | [symbol] | CDZ96939 |
| X₆₉₀₁ | M7 | 79 | 827202 | G | A | [symbol] | CDZ96946 |
| X₆₉₀₂ | M7 | 79 | 853944 | T | A | [symbol] | CDZ96957 |
| X₆₉₀₃ | M7 | 79 | 864204 | C | T | [symbol] | CDZ96959 |
| X₆₉₀₄ | M7 | 79 | 874161 | G | A | [symbol] | CDZ96965 |
| X₆₉₀₅ | M7 | 79 | 928047 | G | A | [symbol] | CDZ96986 |
| X₆₉₀₆ | M7 | 79 | 959497 | C | T | [symbol] | CDZ96997 |
| X₆₉₀₇ | M7 | 79 | 984226 | C | T | [symbol] | CDZ97004 |
| X₆₉₀₈ | M7 | 79 | 990910 | C | T | [symbol] | CDZ97004 |
| X₆₉₀₉ | M7 | 79 | 1000786 | A | G | [symbol] | CDZ97007 |
| X₆₉₁₀ | M7 | 79 | 1061879 | G | A | [symbol] | CDZ97030 |
| X₆₉₁₁ | M7 | 79 | 1069426 | C | T | [symbol] | CDZ97032 |
| X₆₉₁₂ | M7 | 79 | 1082233 | G | A | [symbol] | CDZ97037 |
| X₆₉₁₃ | M7 | 79 | 1117040 | C | T | [symbol] | CDZ97047 |
| X₆₉₁₄ | M7 | 79 | 1123288 | C | T | [symbol] | CDZ97049 |
| X₆₉₁₅ | M7 | 79 | 1140763 | G | A | [symbol] | CDZ97053 |
| X₆₉₁₆ | M7 | 79 | 1167374 | C | T | [symbol] | CDZ97065 |
| X₆₉₁₇ | M7 | 79 | 1229129 | G | A | [symbol] | CDZ97086 |
| X₆₉₁₈ | M7 | 79 | 1231740 | C | T | [symbol] | CDZ97087 |
| X₆₉₁₉ | M7 | 79 | 1279548 | G | A | [symbol] | CDZ97105 |
| X₆₉₂₀ | M7 | 79 | 1287496 | C | T | [symbol] | CDZ97107 |
| X₆₉₂₁ | M7 | 79 | 1318034 | C | T | [symbol] | CDZ97117 |
| X₆₉₂₂ | M7 | 79 | 1336766 | C | T | [symbol] | CDZ97121 |
| X₆₉₂₃ | M7 | 79 | 1344403 | C | T | [symbol] | CDZ97124 |
| X₆₉₂₄ | M7 | 79 | 1367232 | C | T | [symbol] | CDZ97129 |
| X₆₉₂₅ | M7 | 79 | 1430954 | G | A | [symbol] | CDZ97152 |
| X₆₉₂₆ | M7 | 79 | 1540205 | C | T | [symbol] | CDZ97186 |
| X₆₉₂₇ | M7 | 79 | 1541574 | G | A | [symbol] | CDZ97186 |
| X₆₉₂₈ | M7 | 79 | 1556423 | C | T | [symbol] | CDZ97191 |
| X₆₉₂₉ | M7 | 79 | 1624415 | G | A | [symbol] | CDZ97217 |
| X₆₉₃₀ | M7 | 79 | 1658469 | C | T | [symbol] | CDZ97227 |
| X₆₉₃₁ | M7 | 79 | 1674448 | G | A | [symbol] | CDZ97228 |
| X₆₉₃₂ | M7 | 79 | 1708225 | C | T | [symbol] | CDZ97238 |
| X₆₉₃₃ | M7 | 79 | 1797660 | C | T | [symbol] | CDZ97265 |
| X₆₉₃₄ | M7 | 79 | 1797676 | G | A | [symbol] | CDZ97265 |
| X₆₉₃₅ | M7 | 79 | 1842448 | G | A | [symbol] | CDZ97282 |
| X₆₉₃₆ | M7 | 79 | 1855037 | A | G | [symbol] | CDZ97285 |
| X₆₉₃₇ | M7 | 79 | 1859220 | A | G | [symbol] | CDZ97291 |
| X₆₉₃₈ | M7 | 79 | 1977783 | G | A | [symbol] | CDZ97330 |
| X₆₉₃₉ | M7 | 79 | 2013361 | C | T | [symbol] | CDZ97344 |
| X₆₉₄₀ | M7 | 79 | 2027861 | C | T | [symbol] | CDZ97350 |
| X₆₉₄₁ | M7 | 79 | 2055184 | G | A | [symbol] | CDZ97361 |
| X₆₉₄₂ | M7 | 79 | 2121041 | G | A | [symbol] | CDZ97380 |
| X₆₉₄₃ | M7 | 79 | 2152573 | C | T | [symbol] | CDZ97392 |
| X₆₉₄₄ | M7 | 79 | 2172085 | C | T | [symbol] | CDZ97397 |
| X₆₉₄₅ | M7 | 79 | 2188410 | G | A | [symbol] | CDZ97405 |
| X₆₉₄₆ | M7 | 79 | 2268540 | C | T | [symbol] | CDZ97435 |
| X₆₉₄₇ | M7 | 79 | 2339881 | C | G | [symbol] | CDZ97460 |
| X₆₉₄₈ | M7 | 79 | 2340326 | G | A | [symbol] | CDZ97462 |
| X₆₉₄₉ | M7 | 79 | 2340636 | T | C | [symbol] | CDZ97462 |
| X₆₉₅₀ | M7 | 85 | 512 | A | G | [symbol] | |
| X₆₉₅₁ | M7 | 85 | 516 | G | A | [symbol] | |
| X₆₉₅₂ | M7 | 85 | 521 | C | T | [symbol] | |
| X₆₉₅₃ | M7 | 85 | 522 | A | G | [symbol] | |
| X₆₉₅₄ | M7 | 88 | 119 | C | T | [symbol] | CDZ97464 |
| X₆₉₅₅ | M7 | 88 | 196 | G | A | [symbol] | CDZ97464 |
| X₆₉₅₆ | M7 | 88 | 898 | C | T | [symbol] | CDZ97464 |
| X₆₉₅₇ | M7 | 99 | 245 | G | A | [symbol] | |
| X₆₉₅₈ | M7 | 109 | 396 | T | C | [symbol] | |
| X₆₉₅₉ | M7 | 118 | 396 | C | T | [symbol] | CDZ97478 |
| X₆₉₆₀ | M7 | 118 | 870 | G | A | [symbol] | CDZ97478 |
| X₆₉₆₁ | M7 | 118 | 1491 | G | A | [symbol] | CDZ97478 |
| X₆₉₆₂ | M7 | 121 | 1402 | C | G | [symbol] | |
| X₆₉₆₃ | M7 | 124 | 72117 | G | A | [symbol] | CDZ97501 |
| X₆₉₆₄ | M7 | 124 | 72760 | G | A | [symbol] | CDZ97501 |
| X₆₉₆₅ | M7 | 124 | 94611 | G | A | [symbol] | CDZ97507 |
| X₆₉₆₆ | M7 | 124 | 138045 | C | T | [symbol] | CDZ97519 |
| X₆₉₆₇ | M7 | 124 | 156052 | G | A | [symbol] | CDZ97524 |
| X₆₉₆₈ | M7 | 124 | 166988 | C | T | [symbol] | CDZ97527 |
| X₆₉₆₉ | M7 | 124 | 179336 | C | T | [symbol] | CDZ97533 |
| X₆₉₇₀ | M7 | 124 | 222249 | C | T | [symbol] | CDZ97547 |
| X₆₉₇₁ | M7 | 125 | 35864 | T | C | [symbol] | CDZ97573 |
| X₆₉₇₂ | M7 | 129 | 299 | G | A | [symbol] | CDZ97577 |
| X₆₉₇₃ | M7 | 129 | 433 | G | A | [symbol] | CDZ97577 |
| X₆₉₇₄ | M7 | 129 | 856 | G | A | [symbol] | CDZ97577 |
| X₆₉₇₅ | M7 | 129 | 1013 | C | T | [symbol] | CDZ97577 |
| X₆₉₇₆ | M7 | 130 | 674 | C | T | [symbol] | CDZ97578 |
| X₆₉₇₇ | M7 | 138 | 123 | G | T | [symbol] | |
| X₆₉₇₈ | M7 | 141 | 10162 | G | A | [symbol] | CDZ97582 |
| X₆₉₇₉ | M7 | 141 | 14167 | G | A | [symbol] | CDZ97586 |
| X₆₉₈₀ | M7 | 141 | 15764 | C | T | [symbol] | CDZ97587 |
| X₆₉₈₁ | M7 | 141 | 17894 | G | A | [symbol] | CDZ97587 |
| X₆₉₈₂ | M7 | 145 | 296 | A | G | [symbol] | |
| X₆₉₈₃ | M7 | 147 | 335 | C | T | [symbol] | |
| X₆₉₈₄ | M7 | 149 | 2074 | T | G | [symbol] | CDZ97598 |
| X₆₉₈₅ | M7 | 149 | 2453 | C | T | [symbol] | CDZ97598 |
| X₆₉₈₆ | M7 | 159 | 106 | A | C | [symbol] | |
| X₆₉₈₇ | M7 | 162 | 70025 | C | T | [symbol] | CDZ97625 |
| X₆₉₈₈ | M7 | 162 | 70405 | A | G | [symbol] | CDZ97625 |
| X₆₉₈₉ | M7 | 162 | 91190 | C | T | [symbol] | CDZ97631 |
| X₆₉₉₀ | M7 | 162 | 117736 | G | A | [symbol] | CDZ97639 |
| X₆₉₉₁ | M7 | 162 | 118358 | T | C | [symbol] | CDZ97641 |
| X₆₉₉₂ | M7 | 162 | 207179 | C | T | [symbol] | CDZ97668 |
| X₆₉₉₃ | M7 | 162 | 207236 | G | A | [symbol] | CDZ97666 |
| X₆₉₉₄ | M7 | 162 | 223407 | C | T | [symbol] | CDZ97674 |
| X₆₉₉₅ | M7 | 162 | 230526 | C | T | [symbol] | CDZ97676 |
| X₆₉₉₆ | M7 | 162 | 231146 | C | T | [symbol] | CDZ97676 |
| X₆₉₉₇ | M7 | 162 | 233092 | C | T | [symbol] | CDZ97676 |
| X₆₉₉₈ | M7 | 162 | 243753 | G | A | [symbol] | CDZ97680 |
| X₆₉₉₉ | M7 | 162 | 268123 | G | A | [symbol] | CDZ97688 |
| X₇₀₀₀ | M7 | 162 | 291566 | G | A | [symbol] | CDZ97695 |
| X₇₀₀₁ | M7 | 162 | 308369 | T | C | [symbol] | CDZ97702 |
| X₇₀₀₂ | M7 | 162 | 309562 | A | G | [symbol] | CDZ97702 |
| X₇₀₀₃ | M7 | 162 | 321070 | C | T | [symbol] | CDZ97703 |
| X₇₀₀₄ | M7 | 162 | 321073 | A | G | [symbol] | CDZ97703 |
| X₇₀₀₅ | M7 | 162 | 321460 | C | G | [symbol] | CDZ97703 |
| X₂₀₀₆ | M7 | 162 | 321463 | A | C | [symbol] | CDZ97703 |
| X₇₀₀₇ | M7 | 162 | 321975 | T | G | [symbol] | CDZ97703 |
| X₇₀₀₈ | M7 | 162 | 322591 | G | A | [symbol] | CDZ97703 |
| X₇₀₀₉ | M7 | 162 | 323480 | T | C | [symbol] | CDZ97703 |
| X₇₀₁₀ | M7 | 162 | 324114 | T | C | [symbol] | CDZ97703 |
| X₇₀₁₁ | M7 | 162 | 324139 | C | T | [symbol] | CDZ97703 |
| X₇₀₁₂ | M7 | 162 | 324568 | A | G | [symbol] | CDZ97703 |
| X₇₀₁₃ | M7 | 162 | 360320 | G | A | [symbol] | CDZ97714 |
| X₇₀₁₄ | M7 | 162 | 408274 | C | T | [symbol] | CDZ97733 |
| X₇₀₁₅ | M7 | 162 | 424391 | C | T | [symbol] | CDZ97740 |
| X₇₀₁₆ | M7 | 162 | 444123 | T | C | [symbol] | CDZ97748 |
| X₇₀₁₇ | M7 | 162 | 467207 | C | T | [symbol] | CDZ97755 |
| X₇₀₁₈ | M7 | 162 | 476928 | C | T | [symbol] | CDZ97760 |
| X₇₀₁₉ | M7 | 162 | 491906 | G | A | [symbol] | CDZ97764 |
| X₇₀₂₀ | M7 | 162 | 492101 | G | A | [symbol] | CDZ97764 |
| X₇₀₂₁ | M7 | 162 | 501886 | C | T | [symbol] | CDZ97769 |
| X₇₀₂₂ | M7 | 162 | 581141 | C | A | [symbol] | CDZ97795 |
| X₇₀₂₃ | M7 | 162 | 632311 | G | A | [symbol] | CDZ97814 |
| X₇₀₂₄ | M7 | 162 | 633321 | G | A | [symbol] | CDZ97814 |
| X₇₀₂₅ | M7 | 162 | 633633 | G | A | [symbol] | CDZ97814 |
| X₇₀₂₆ | M7 | 162 | 640005 | C | T | [symbol] | CDZ97815 |
| X₇₀₂₇ | M7 | 162 | 680645 | G | A | [symbol] | CDZ97826 |
| X₇₀₂₈ | M7 | 162 | 721840 | G | A | [symbol] | CDZ97838 |
| X₇₀₂₉ | M7 | 162 | 738675 | G | A | [symbol] | CDZ97842 |
| X₇₀₃₀ | M7 | 162 | 738686 | C | T | [symbol] | CDZ97842 |
| X₇₀₃₁ | M7 | 162 | 801198 | C | T | [symbol] | CDZ97864 |
| X₇₀₃₂ | M7 | 162 | 808830 | G | A | [symbol] | CDZ97869 |
| X₇₀₃₃ | M7 | 162 | 871911 | C | T | [symbol] | CDZ97890 |
| X₇₀₃₄ | M7 | 162 | 946623 | C | T | [symbol] | CDZ97916 |
| X₇₀₃₅ | M7 | 162 | 971882 | G | A | [symbol] | CDZ97924 |
| X₇₀₃₆ | M7 | 163 | 225 | G | A | [symbol] | |
| X₇₀₃₇ | M7 | 169 | 1056 | C | T | [symbol] | |
| X₇₀₃₈ | M7 | 189 | 126573 | G | A | [symbol] | CDZ97996 |
| X₇₀₃₉ | M7 | 189 | 127356 | G | A | [symbol] | CDZ97996 |
| X₇₀₄₀ | M7 | 189 | 174918 | G | A | [symbol] | CDZ98015 |
| X₇₀₄₁ | M7 | 189 | 194647 | A | G | [symbol] | CDZ98020 |
| X₇₀₄₂ | M7 | 189 | 194665 | A | G | [symbol] | CDZ98020 |
| X₇₀₄₃ | M7 | 189 | 211865 | C | T | [symbol] | CDZ98028 |
| X₇₀₄₄ | M7 | 189 | 212408 | G | A | [symbol] | CDZ98028 |
| X₇₀₄₅ | M7 | 189 | 212649 | C | T | [symbol] | CDZ98028 |
| X₇₀₄₆ | M7 | 189 | 273599 | C | A | [symbol] | CDZ98048 |
| X₇₀₄₇ | M7 | 189 | 323680 | C | T | [symbol] | CDZ98063 |
| X₇₀₄₈ | M7 | 189 | 332957 | G | A | [symbol] | CDZ98065 |
| X₇₀₄₉ | M7 | 189 | 371247 | G | A | [symbol] | CDZ98078 |
| X₇₀₅₀ | M7 | 191 | 342 | A | G | [symbol] | CDZ98107 |
| X₇₀₅₁ | M7 | 191 | 347 | C | T | [symbol] | CDZ98107 |
| X₇₀₅₂ | M7 | 191 | 427 | C | T | [symbol] | CDZ98107 |
| X₇₀₅₃ | M7 | 191 | 451 | C | T | [symbol] | CDZ98107 |
| X₇₀₅₄ | M7 | 191 | 487 | A | G | [symbol] | CDZ98107 |
| X₇₀₅₅ | M7 | 197 | 624 | C | T | [symbol] | CDZ98111 |
| X₇₀₅₆ | M7 | 198 | 202 | C | A | [symbol] | |
| X₇₀₅₇ | M7 | 198 | 203 | G | A | [symbol] | |
| X₇₀₅₈ | M7 | 198 | 238 | C | T | [symbol] | |
| X₇₀₅₉ | M7 | 198 | 265 | C | T | [symbol] | |
| X₇₀₆₀ | M7 | 199 | 688 | C | T | [symbol] | |
| X₇₀₆₁ | M7 | 199 | 911 | C | T | [symbol] | |
| X₇₀₆₂ | M7 | 199 | 971 | C | T | [symbol] | |
| X₇₀₆₃ | M7 | 199 | 1218 | C | T | [symbol] | |
| X₇₀₆₄ | M7 | 202 | 171 | C | T | [symbol] | |
| X₇₀₆₅ | M7 | 202 | 221 | C | T | [symbol] | |
| X₇₀₆₆ | M7 | 205 | 360 | C | T | [symbol] | CDZ98112 |
| X₇₀₆₇ | M7 | 205 | 981 | C | T | [symbol] | CDZ98112 |
| X₇₀₆₈ | M7 | 206 | 400 | G | A | [symbol] | |
| X₇₀₆₉ | M7 | 208 | 117 | G | A | [symbol] | |
| X₇₀₇₀ | M7 | 208 | 476 | G | A | [symbol] | |
| X₇₀₇₁ | M7 | 208 | 616 | G | A | [symbol] | |
| X₇₀₇₂ | M7 | 208 | 935 | G | A | [symbol] | |
| X₇₀₇₃ | M7 | 209 | 312 | C | T | [symbol] | CDZ98113 |
| X₇₀₇₄ | M7 | 223 | 93 | G | A | [symbol] | CDZ98118 |
| X₇₀₇₅ | M7 | 223 | 102 | A | G | [symbol] | CDZ98118 |
| X₇₀₇₆ | M7 | 223 | 133 | A | G | [symbol] | CDZ98118 |
| X₇₀₇₇ | M7 | 223 | 150 | T | C | [symbol] | CDZ98118 |
| X₇₀₇₈ | M7 | 223 | 155 | A | C | [symbol] | CDZ98118 |
| X₇₀₇₉ | M7 | 223 | 158 | T | C | [symbol] | CDZ98118 |
| X₇₀₈₀ | M7 | 223 | 160 | C | T | [symbol] | CDZ98118 |
| X₇₀₈₁ | M7 | 223 | 175 | G | A | [symbol] | CDZ98118 |
| X₇₀₈₂ | M7 | 223 | 177 | A | G | [symbol] | CDZ98118 |
| X₇₀₈₃ | M7 | 223 | 188 | A | G | [symbol] | CDZ98118 |
| X₇₀₈₄ | M7 | 223 | 192 | C | A | [symbol] | CDZ98118 |
| X₇₀₈₅ | M7 | 223 | 194 | T | C | [symbol] | CDZ98118 |
| X₇₀₈₆ | M7 | 223 | 195 | T | G | [symbol] | CDZ98118 |
| X₇₀₈₇ | M7 | 223 | 197 | T | C | [symbol] | CDZ98118 |
| X₇₀₈₈ | M7 | 223 | 202 | G | C | [symbol] | CDZ98118 |
| X₇₀₈₉ | M7 | 223 | 205 | C | T | [symbol] | CDZ98118 |
| X₇₀₉₀ | M7 | 225 | 317 | C | T | [symbol] | |
| X₇₀₉₁ | M7 | 227 | 202 | G | A | [symbol] | |
| X₇₀₉₂ | M7 | 232 | 340 | G | A | [symbol] | CDZ98119 |
| X₇₀₉₃ | M7 | 232 | 415 | G | A | [symbol] | CDZ98119 |
| X₇₀₉₄ | M7 | 232 | 1379 | C | T | [symbol] | CDZ98119 |
| X₇₀₉₅ | M7 | 232 | 1521 | C | T | [symbol] | CDZ98119 |
| X₇₀₉₆ | M7 | 232 | 1662 | G | A | [symbol] | CDZ98119 |
| X₇₀₉₇ | M7 | 232 | 1761 | C | T | [symbol] | CDZ98119 |
| X₇₀₉₈ | M7 | 232 | 2027 | C | T | [symbol] | CDZ98119 |
| X₇₀₉₉ | M7 | 232 | 2028 | C | T | [symbol] | CDZ98119 |
| X₇₁₀₀ | M7 | 232 | 3060 | C | T | [symbol] | CDZ98120 |
| X₇₁₀₁ | M7 | 232 | 3284 | C | T | [symbol] | CDZ98119 |
| X₇₁₀₂ | M7 | 232 | 4437 | G | A | [symbol] | CDZ98121 |
| X₇₁₀₃ | M7 | 232 | 4561 | C | T | [symbol] | CDZ98120 |
| X₇₁₀₄ | M7 | 242 | 7334 | C | T | [symbol] | CDZ98125 |
| X₇₁₀₅ | M7 | 242 | 41710 | G | A | [symbol] | CDZ98131 |
| X₇₁₀₆ | M7 | 242 | 50262 | G | A | [symbol] | CDZ98135 |
| X₇₁₀₇ | M7 | 242 | 84132 | G | A | [symbol] | CDZ98146 |
| X₇₁₀₈ | M7 | 242 | 88037 | G | A | [symbol] | CDZ98147 |
| X₇₁₀₉ | M7 | 242 | 106805 | G | A | [symbol] | CDZ98152 |
| X₇₁₁₀ | M7 | 242 | 107587 | G | A | [symbol] | CDZ98153 |
| X₇₁₁₁ | M7 | 242 | 125424 | C | T | [symbol] | CDZ98159 |
| X₇₁₁₂ | M7 | 242 | 168689 | G | A | [symbol] | CDZ98172 |
| X₇₁₁₃ | M7 | 242 | 213986 | G | A | [symbol] | CDZ98185 |
| X₇₁₁₄ | M7 | 242 | 227271 | C | T | [symbol] | CDZ98191 |
| X₇₁₁₅ | M7 | 242 | 233088 | G | A | [symbol] | CDZ98193 |
| X₇₁₁₆ | M7 | 242 | 310353 | G | A | [symbol] | CDZ98216 |
| X₇₁₁₇ | M7 | 242 | 374897 | G | A | [symbol] | CDZ98238 |
| X₇₁₁₈ | M7 | 242 | 404132 | G | A | [symbol] | CDZ98248 |
| X₇₁₁₉ | M7 | 242 | 412676 | G | A | [symbol] | CDZ98249 |
| X₇₁₂₀ | M7 | 242 | 454709 | C | T | [symbol] | CDZ98263 |
| X₇₁₂₁ | M7 | 242 | 472562 | C | T | [symbol] | CDZ98266 |
| X₇₁₂₂ | M7 | 242 | 485936 | G | A | [symbol] | CDZ98270 |
| X₇₁₂₃ | M7 | 242 | 492389 | G | A | [symbol] | CDZ98274 |
| X₇₁₂₄ | M7 | 242 | 514780 | C | T | [symbol] | CDZ98282 |
| X₇₁₂₅ | M7 | 242 | 528340 | G | A | [symbol] | CDZ98288 |
| X₇₁₂₆ | M7 | 242 | 542663 | G | A | [symbol] | CDZ98290 |
| X₇₁₂₇ | M7 | 242 | 559999 | G | A | [symbol] | CDZ98297 |
| X₇₁₂₈ | M7 | 242 | 602456 | G | A | [symbol] | CDZ98308 |
| X₇₁₂₉ | M7 | 242 | 602531 | G | A | [symbol] | CDZ98308 |
| X₇₁₃₀ | M7 | 242 | 603042 | G | A | [symbol] | CDZ98308 |
| X₇₁₃₁ | M7 | 242 | 604704 | C | T | [symbol] | CDZ98309 |
| X₇₁₃₂ | M7 | 242 | 628488 | G | A | [symbol] | CDZ98317 |
| X₇₁₃₃ | M7 | 242 | 641669 | G | A | [symbol] | CDZ98323 |
| X₇₁₃₄ | M7 | 249 | 206 | G | A | [symbol] | CED84929 |
| X₇₁₃₅ | M7 | 249 | 42295 | C | T | [symbol] | CED84942 |
| X₇₁₃₆ | M7 | 249 | 52397 | G | A | [symbol] | CED84947 |
| X₇₁₃₇ | M7 | 249 | 72016 | C | T | [symbol] | CED84950 |
| X₇₁₃₈ | M7 | 249 | 141163 | A | G | [symbol] | CED84977 |
| X₇₁₃₉ | M7 | 249 | 157550 | C | T | [symbol] | CED84983 |
| X₇₁₄₀ | M7 | 249 | 191304 | C | T | [symbol] | CED84988 |
| X₇₁₄₁ | M7 | 249 | 269707 | G | A | [symbol] | CED85013 |
| X₇₁₄₂ | M7 | 249 | 272075 | C | T | [symbol] | CED85013 |
| X₇₁₄₃ | M7 | 249 | 285231 | C | T | [symbol] | CED85015 |
| X₇₁₄₄ | M7 | 249 | 289154 | C | T | [symbol] | CED85018 |
| X₇₁₄₅ | M7 | 249 | 305975 | G | A | [symbol] | CED85023 |
| X₇₁₄₆ | M7 | 249 | 342990 | G | A | [symbol] | CED85036 |
| X₇₁₄₇ | M7 | 249 | 377814 | G | A | [symbol] | CED85051 |
| X₇₁₄₈ | M7 | 249 | 390024 | G | A | [symbol] | CED85059 |
| X₇₁₄₉ | M7 | 249 | 434431 | G | A | [symbol] | CED85072 |
| X₇₁₅₀ | M7 | 249 | 485782 | C | T | [symbol] | CED85089 |
| X₇₁₅₁ | M7 | 249 | 488584 | G | A | [symbol] | CED85091 |
| X₇₁₅₂ | M7 | 249 | 501310 | C | T | [symbol] | CED85093 |
| X₇₁₅₃ | M7 | 249 | 506067 | A | G | [symbol] | CED85094 |
| X₇₁₅₄ | M7 | 249 | 509901 | C | T | [symbol] | CED85096 |
| X₇₁₅₅ | M7 | 249 | 532943 | A | G | [symbol] | CED85099 |
| X₇₁₅₆ | M7 | 249 | 574798 | C | T | [symbol] | CED85117 |
| X₇₁₅₇ | M7 | 249 | 598579 | C | T | [symbol] | CED85125 |
| X₇₁₅₈ | M7 | 249 | 626627 | G | A | [symbol] | CED85132 |
| X₇₁₅₉ | M7 | 249 | 637944 | G | A | [symbol] | CED85135 |
| X₇₁₆₀ | M7 | 249 | 660408 | G | A | [symbol] | CED85144 |
| X₇₁₆₁ | M7 | 249 | 665383 | G | A | [symbol] | CED85144 |
| X₇₁₆₂ | M7 | 249 | 683254 | T | C | [symbol] | CED85153 |
| X₇₁₆₃ | M7 | 249 | 728713 | T | C | [symbol] | CED85167 |
| X₇₁₆₄ | M7 | 249 | 746655 | C | T | [symbol] | CED85172 |
| X₇₁₆₅ | M7 | 249 | 751427 | G | A | [symbol] | CED85174 |
| X₇₁₆₆ | M7 | 249 | 803215 | C | T | [symbol] | CED85186 |
| X₇₁₆₇ | M7 | 249 | 823733 | C | T | [symbol] | CED85193 |
| X₇₁₆₈ | M7 | 249 | 854220 | G | A | [symbol] | CED85201 |
| X₇₁₆₉ | M7 | 249 | 911274 | C | T | [symbol] | CED85218 |
| X₇₁₇₀ | M7 | 249 | 932606 | C | T | [symbol] | CED85223 |
| X₇₁₇₁ | M7 | 249 | 945434 | C | T | [symbol] | CED85227 |
| X₇₁₇₂ | M7 | 249 | 945727 | C | T | [symbol] | CED85227 |
| X₇₁₇₃ | M7 | 249 | 953534 | A | T | [symbol] | CED85229 |
| X₇₁₇₄ | M7 | 249 | 969333 | G | T | [symbol] | CED85238 |
| X₇₁₇₅ | M7 | 249 | 977170 | C | T | [symbol] | CED85242 |
| X₇₁₇₆ | M7 | 249 | 989520 | C | T | [symbol] | CED85243 |
| X₇₁₇₇ | M7 | 249 | 1014098 | C | T | [symbol] | CED85252 |
| X₇₁₇₈ | M7 | 249 | 1058697 | C | T | [symbol] | CED85263 |
| X₇₁₇₉ | M7 | 249 | 1075522 | G | A | [symbol] | CED85270 |
| X₇₁₈₀ | M7 | 249 | 1114790 | G | A | [symbol] | CED85283 |
| X₇₁₈₁ | M7 | 249 | 1144309 | G | A | [symbol] | CED85291 |
| X₇₁₈₂ | M7 | 249 | 1176977 | G | A | [symbol] | CED85303 |
| X₇₁₈₃ | M7 | 249 | 1195367 | G | A | [symbol] | CED85307 |
| X₇₁₈₄ | M7 | 249 | 1209489 | C | T | [symbol] | CED85313 |
| X₇₁₈₅ | M7 | 249 | 1230068 | C | T | [symbol] | CED85319 |
| X₇₁₈₆ | M7 | 249 | 1245242 | G | A | [symbol] | CED85322 |
| X₇₁₈₇ | M7 | 249 | 1299472 | C | T | [symbol] | CED85343 |
| X₇₁₈₈ | M7 | 249 | 1305184 | G | A | [symbol] | CED85345 |
| X₇₁₈₉ | M7 | 249 | 1350079 | G | A | [symbol] | CED85357 |
| X₇₁₉₀ | M7 | 249 | 1391343 | G | A | [symbol] | CED85372 |
| X₇₁₉₁ | M7 | 249 | 1452855 | C | T | [symbol] | CED85396 |
| X₇₁₉₂ | M7 | 249 | 1486813 | A | C | [symbol] | CED85411 |
| X₇₁₉₃ | M7 | 249 | 1486828 | G | A | [symbol] | CED85411 |
| X₇₁₉₄ | M7 | 249 | 1486831 | T | C | [symbol] | CED85411 |
| X₇₁₉₅ | M7 | 249 | 1498029 | T | C | [symbol] | CED85415 |
| X₇₁₉₆ | M7 | 249 | 1498823 | T | C | [symbol] | CED85414 |
| X₇₁₉₇ | M7 | 249 | 1498829 | C | T | [symbol] | CED85414 |
| X₇₁₉₈ | M7 | 249 | 1499357 | T | G | [symbol] | CED85415 |
| X₇₁₉₉ | M7 | 249 | 1499741 | G | A | [symbol] | CED85415 |
| X₇₂₀₀ | M7 | 249 | 1502631 | G | T | [symbol] | CED85416 |
| X₇₂₀₁ | M7 | 249 | 1502641 | T | A | [symbol] | CED85416 |
| X₇₂₀₂ | M7 | 249 | 1502819 | T | G | [symbol] | CED85416 |
| X₇₂₀₃ | M7 | 249 | 1505138 | G | A | [symbol] | CED85416 |
| X₇₂₀₄ | M7 | 249 | 1518591 | T | A | [symbol] | CED85419 |
| X₇₂₀₅ | M7 | 249 | 1519431 | A | G | [symbol] | CED85419 |
| X₇₂₀₆ | M7 | 249 | 1553019 | G | A | [symbol] | CED85427 |
| X₇₂₀₇ | M7 | 249 | 1599808 | C | T | [symbol] | CED85444 |
| X₇₂₀₈ | M7 | 249 | 1599907 | C | T | [symbol] | CED85444 |
| X₇₂₀₉ | M7 | 249 | 1619036 | G | A | [symbol] | CED85450 |
| X₇₂₁₀ | M7 | 249 | 1621516 | G | A | [symbol] | CED85451 |
| X₇₂₁₁ | M7 | 249 | 1623151 | G | A | [symbol] | CED85451 |
| X₇₂₁₂ | M7 | 249 | 1636931 | G | A | [symbol] | CED85455 |
| X₇₂₁₃ | M7 | 249 | 1643086 | T | A | [symbol] | CED85456 |
| X₇₂₁₄ | M7 | 249 | 1653615 | T | G | [symbol] | CED85457 |
| X₇₂₁₅ | M7 | 249 | 1662746 | A | T | [symbol] | CED85459 |
| X₇₂₁₆ | M7 | 249 | 1666185 | C | T | [symbol] | CED85460 |
| X₇₂₁₇ | M7 | 249 | 1679615 | G | A | [symbol] | CED85461 |
| X₇₂₁₈ | M7 | 249 | 1726777 | C | T | [symbol] | CED85474 |
| X₇₂₁₉ | M7 | 249 | 1737431 | C | T | [symbol] | CED85478 |
| X₇₂₂₀ | M7 | 249 | 1741408 | C | T | [symbol] | CED85480 |
| X₇₂₂₁ | M7 | 249 | 1760715 | C | T | [symbol] | CED85489 |
| X₇₂₂₂ | M7 | 249 | 1768844 | C | T | [symbol] | CED85489 |
| X₇₂₂₃ | M7 | 249 | 1780551 | A | G | [symbol] | CED85493 |
| X₇₂₂₄ | M7 | 249 | 1780569 | A | G | [symbol] | CED85493 |
| X₇₂₂₅ | M7 | 249 | 1786050 | C | T | [symbol] | CED85496 |
| X₇₂₂₆ | M7 | 249 | 1803575 | C | T | [symbol] | CED85502 |
| X₇₂₂₇ | M7 | 249 | 1862072 | C | T | [symbol] | CED85520 |
| X₇₂₂₈ | M7 | 249 | 1880762 | G | A | [symbol] | CED85525 |
| X₇₂₂₉ | M7 | 249 | 1944026 | G | A | [symbol] | CED85545 |
| X₇₂₃₀ | M7 | 249 | 1969421 | G | A | [symbol] | CED85550 |
| X₇₂₃₁ | M7 | 249 | 1988505 | C | T | [symbol] | CED85560 |
| X₇₂₃₂ | M7 | 249 | 2010712 | G | A | [symbol] | CED85564 |
| X₇₂₃₃ | M7 | 249 | 2015272 | G | A | [symbol] | CED85567 |
| X₇₂₃₄ | M7 | 249 | 2027477 | G | A | [symbol] | CED85573 |
| X₇₂₃₅ | M7 | 249 | 2054064 | C | T | [symbol] | CED85583 |
| X₇₂₃₆ | M7 | 249 | 2063467 | G | A | [symbol] | CED85586 |
| X₇₂₃₇ | M7 | 249 | 2080239 | G | A | [symbol] | CED85590 |
| X₇₂₃₈ | M7 | 249 | 2110956 | C | T | [symbol] | CED85603 |
| X₇₂₃₉ | M7 | 249 | 2124517 | C | T | [symbol] | CED85607 |
| X₇₂₄₀ | M7 | 249 | 2131353 | C | T | [symbol] | CED85609 |
| X₇₂₄₁ | M7 | 249 | 2167664 | G | A | [symbol] | CED85620 |
| X₇₂₄₂ | M7 | 249 | 2178506 | G | A | [symbol] | CED85622 |
| X₇₂₄₃ | M7 | 249 | 2183695 | G | A | [symbol] | CED85623 |
| X₇₂₄₄ | M7 | 249 | 2198205 | G | A | [symbol] | CED85628 |
| X₇₂₄₅ | M7 | 249 | 2223626 | G | A | [symbol] | CED85633 |
| X₇₂₄₆ | M7 | 249 | 2255547 | G | C | [symbol] | CED85640 |
| X₇₂₄₇ | M7 | 252 | 512 | C | T | [symbol] | |
| X₇₂₄₈ | M7 | 252 | 524 | C | T | [symbol] | |
| X₇₂₄₉ | M7 | 258 | 267 | G | A | [symbol] | |
| X₇₂₅₀ | M7 | 258 | 1139 | A | T | [symbol] | |
| X₇₂₅₁ | M7 | 259 | 91 | G | C | [symbol] | |
| X₇₂₅₂ | M7 | 260 | 156 | G | A | [symbol] | |
| X₇₂₅₃ | M7 | 262 | 4049 | G | A | [symbol] | CDZ98330 |
| X₇₂₅₄ | M7 | 262 | 12289 | C | T | [symbol] | CDZ98333 |
| X₇₂₅₅ | M7 | 262 | 16863 | C | T | [symbol] | CDZ98334 |
| X₇₂₅₆ | M7 | 262 | 30347 | G | A | [symbol] | CDZ98338 |
| X₇₂₅₇ | M7 | 262 | 30359 | G | A | [symbol] | CDZ98338 |
| X₇₂₅₈ | M7 | 262 | 39621 | C | T | [symbol] | CDZ98342 |
| X₇₂₅₉ | M7 | 262 | 96076 | C | T | [symbol] | CDZ98364 |
| X₇₂₆₀ | M7 | 262 | 138889 | C | T | [symbol] | CDZ98375 |
| X₇₂₆₁ | M7 | 262 | 143387 | C | T | [symbol] | CDZ98375 |
| X₇₂₆₂ | M7 | 262 | 144566 | G | A | [symbol] | CDZ98375 |
| X₇₂₆₃ | M7 | 262 | 148151 | C | T | [symbol] | CDZ98375 |
| X₇₂₆₄ | M7 | 262 | 164438 | G | A | [symbol] | CDZ98383 |
| X₇₂₆₅ | M7 | 262 | 201531 | G | A | [symbol] | CDZ98396 |
| X₇₂₆₆ | M7 | 262 | 250756 | A | G | [symbol] | CDZ98412 |
| X₇₂₆₇ | M7 | 262 | 252708 | G | A | [symbol] | CDZ98414 |
| X₇₂₆₈ | M7 | 262 | 319302 | G | A | [symbol] | CDZ98436 |
| X₇₂₆₉ | M7 | 262 | 345538 | C | T | [symbol] | CDZ98448 |
| X₇₂₇₀ | M7 | 262 | 380519 | C | T | [symbol] | CDZ98457 |
| X₇₂₇₁ | M7 | 262 | 383215 | C | T | [symbol] | CDZ98459 |
| X₇₂₇₂ | M7 | 262 | 392930 | C | T | [symbol] | CDZ98461 |
| X₇₂₇₃ | M7 | 262 | 397539 | G | A | [symbol] | CDZ98463 |
| X₇₂₇₄ | M7 | 262 | 410688 | C | T | [symbol] | CDZ98466 |
| X₇₂₇₅ | M7 | 262 | 415802 | C | T | [symbol] | CDZ98467 |
| X₇₂₇₆ | M7 | 262 | 420102 | G | A | [symbol] | CDZ98468 |
| X₇₂₇₇ | M7 | 262 | 444331 | G | A | [symbol] | CDZ98477 |
| X₇₂₇₈ | M7 | 262 | 446956 | C | T | [symbol] | CDZ98477 |
| X₇₂₇₉ | M7 | 262 | 460607 | C | A | [symbol] | CDZ98482 |
| X₇₂₈₀ | M7 | 262 | 503840 | G | A | [symbol] | CDZ98499 |
| X₇₂₈₁ | M7 | 262 | 539428 | G | A | [symbol] | CDZ98514 |
| X₇₂₈₂ | M7 | 262 | 539452 | A | G | [symbol] | CDZ98514 |
| X₇₂₈₃ | M7 | 262 | 539470 | A | G | [symbol] | CDZ98514 |
| X₇₂₈₄ | M7 | 262 | 557280 | G | A | [symbol] | CDZ98518 |
| X₇₂₈₅ | M7 | 262 | 560713 | G | A | [symbol] | CDZ98519 |
| X₇₂₈₆ | M7 | 262 | 562795 | C | T | [symbol] | CDZ98521 |
| X₇₂₈₇ | M7 | 262 | 567591 | G | A | [symbol] | CDZ98524 |
| X₇₂₈₈ | M7 | 262 | 567801 | G | A | [symbol] | CDZ98524 |
| X₇₂₈₉ | M7 | 262 | 577776 | C | T | [symbol] | CDZ98526 |
| X₇₂₉₀ | M7 | 262 | 607661 | G | A | [symbol] | CDZ98533 |
| X₇₂₉₁ | M7 | 262 | 618198 | G | A | [symbol] | CDZ98538 |
| X₇₂₉₂ | M7 | 262 | 639869 | G | C | [symbol] | CDZ98544 |
| X₇₂₉₃ | M7 | 262 | 639962 | A | G | [symbol] | CDZ98544 |
| X₇₂₉₄ | M7 | 262 | 640066 | A | G | [symbol] | CDZ98544 |
| X₇₂₉₅ | M7 | 262 | 640111 | A | G | [symbol] | CDZ98544 |
| X₇₂₉₆ | M7 | 262 | 644677 | C | T | [symbol] | CDZ98544- |
| | | | | | | | CDZ98545 |
| X₇₂₉₇ | M7 | 262 | 647379 | A | G | [symbol] | CDZ98544- |
| | | | | | | | CDZ98545 |
| X₇₂₉₈ | M7 | 262 | 647388 | T | C | [symbol] | CDZ98544- |
| | | | | | | | CDZ98545 |
| X₇₂₉₉ | M7 | 262 | 647393 | G | A | [symbol] | CDZ98544- |
| | | | | | | | CDZ98545 |
| X₇₃₀₀ | M7 | 262 | 647394 | A | G | [symbol] | CDZ98544- |
| | | | | | | | CDZ98545 |
| X₇₃₀₁ | M7 | 262 | 647430 | C | T | [symbol] | CDZ98544- |
| | | | | | | | CDZ98545 |
| X₇₃₀₂ | M7 | 262 | 647433 | A | G | [symbol] | CDZ98544- |
| | | | | | | | CDZ98545 |
| X₇₃₀₃ | M7 | 262 | 653920 | A | G | [symbol] | CDZ98545 |
| X₇₃₀₄ | M7 | 262 | 656031 | C | A | [symbol] | CDZ98545 |
| X₇₃₀₅ | M7 | 262 | 656323 | C | G | [symbol] | CDZ98545 |
| X₇₃₀₆ | M7 | 262 | 668764 | C | T | [symbol] | CDZ98545 |
| X₇₃₀₇ | M7 | 262 | 669984 | C | T | [symbol] | CDZ98547 |
| X₇₃₀₈ | M7 | 262 | 691577 | C | T | [symbol] | CDZ98551 |
| X₇₃₀₉ | M7 | 262 | 759057 | C | A | [symbol] | CDZ98575 |
| X₇₃₁₀ | M7 | 262 | 759058 | A | C | [symbol] | CDZ98575 |
| X₇₃₁₁ | M7 | 262 | 801565 | G | A | [symbol] | CDZ98589 |
| X₇₃₁₂ | M7 | 262 | 822734 | G | A | [symbol] | CDZ98597 |
| X₇₃₁₃ | M7 | 262 | 835874 | C | T | [symbol] | CDZ98601 |
| X₇₃₁₄ | M7 | 262 | 856616 | G | A | [symbol] | CDZ98612 |
| X₇₃₁₅ | M7 | 262 | 865999 | G | A | [symbol] | CDZ98613 |
| X₇₃₁₆ | M7 | 262 | 882258 | G | A | [symbol] | CDZ98619 |
| X₇₃₁₇ | M7 | 262 | 971713 | G | A | [symbol] | CDZ98649 |
| X₇₃₁₈ | M7 | 262 | 1008174 | C | T | [symbol] | CDZ98659 |
| X₇₃₁₉ | M7 | 262 | 1021485 | C | T | [symbol] | CDZ98663 |
| X₇₃₂₀ | M7 | 262 | 1080137 | G | A | [symbol] | CDZ98679 |
| X₇₃₂₁ | M7 | 262 | 1120617 | G | A | [symbol] | CDZ98696 |
| X₇₃₂₂ | M7 | 262 | 1204782 | G | A | [symbol] | CDZ98722 |
| X₇₃₂₃ | M7 | 262 | 1209662 | G | A | [symbol] | CDZ98723 |
| X₇₃₂₄ | M7 | 262 | 1226072 | C | T | [symbol] | CDZ98730 |
| X₇₃₂₅ | M7 | 262 | 1229407 | G | A | [symbol] | CDZ98732 |
| X₇₃₂₆ | M7 | 262 | 1277604 | G | A | [symbol] | CDZ98747 |
| X₇₃₂₇ | M7 | 262 | 1305361 | G | A | [symbol] | CDZ98753 |
| X₇₃₂₈ | M7 | 262 | 1329643 | C | T | [symbol] | CDZ98760 |
| X₇₃₂₉ | M7 | 262 | 1332176 | C | T | [symbol] | CDZ98761 |
| X₇₃₃₀ | M7 | 262 | 1349561 | G | A | [symbol] | CDZ98766 |
| X₇₃₃₁ | M7 | 262 | 1351116 | A | G | [symbol] | CDZ98766 |
| X₇₃₃₂ | M7 | 262 | 1367361 | C | T | [symbol] | CDZ98772 |
| X₇₃₃₃ | M7 | 262 | 1408928 | G | A | [symbol] | CDZ98786 |
| X₇₃₃₄ | M7 | 262 | 1416604 | C | T | [symbol] | CDZ98788 |
| X₇₃₃₅ | M7 | 262 | 1424293 | G | A | [symbol] | CDZ98791 |
| X₇₃₃₆ | M7 | 262 | 1455677 | G | A | [symbol] | CDZ98804 |
| X₇₃₃₇ | M7 | 262 | 1458863 | C | T | [symbol] | CDZ98807 |
| X₇₃₃₈ | M7 | 262 | 1550311 | C | T | [symbol] | CDZ98836 |
| X₇₃₃₉ | M7 | 262 | 1550456 | C | T | [symbol] | CDZ98837 |
| X₇₃₄₀ | M7 | 262 | 1615762 | G | A | [symbol] | CDZ98855 |
| X₇₃₄₁ | M7 | 262 | 1677620 | G | A | [symbol] | CDZ98873 |
| X₇₃₄₂ | M7 | 262 | 1694625 | C | T | [symbol] | CDZ98878 |
| X₇₃₄₃ | M7 | 262 | 1704629 | T | C | [symbol] | CDZ98883 |
| X₇₃₄₄ | M7 | 262 | 1706076 | G | A | [symbol] | CDZ98885 |
| X₇₃₄₅ | M7 | 262 | 1709988 | G | A | [symbol] | CDZ98884 |
| X₇₃₄₆ | M7 | 262 | 1711527 | G | A | [symbol] | CDZ98886 |
| X₇₃₄₇ | M7 | 262 | 1716385 | G | A | [symbol] | CDZ98887 |
| X₇₃₄₈ | M7 | 266 | 252 | C | T | [symbol] | |
| X₇₃₄₉ | M7 | 267 | 93 | G | A | [symbol] | |
| X₇₃₅₀ | M7 | 267 | 94 | A | G | [symbol] | |
| X₇₃₅₁ | M7 | 267 | 133 | T | C | [symbol] | |
| X₇₃₅₂ | M7 | 267 | 134 | C | T | [symbol] | |
| X₇₃₅₃ | M7 | 267 | 201 | T | C | [symbol] | |
| X₇₃₅₄ | M7 | 267 | 208 | T | C | [symbol] | |
| X₇₃₅₅ | M7 | 267 | 234 | T | C | [symbol] | |
| X₇₃₅₆ | M7 | 267 | 254 | G | T | [symbol] | |
| X₇₃₅₇ | M7 | 267 | 256 | A | G | [symbol] | |
| X₇₃₅₈ | M7 | 267 | 257 | T | C | [symbol] | |
| X₇₃₅₉ | M8 | 22 | 1643 | C | T | [symbol] | CED80056 |
| X₇₃₆₀ | M8 | 22 | 2524 | G | A | [symbol] | CED80056 |
| X₇₃₆₁ | M8 | 28 | 236 | G | A | [symbol] | CDZ96150 |
| X₇₃₆₂ | M8 | 28 | 377 | G | A | [symbol] | CDZ96150 |
| X₇₃₆₃ | M8 | 28 | 1506995 | C | T | [symbol] | CDZ96150 |
| X₇₃₆₄ | M8 | 28 | 1677 | C | T | [symbol] | CDZ96150 |
| X₇₃₆₅ | M8 | 43 | 464 | G | A | [symbol] | CDZ96151 |
| X₇₃₆₆ | M8 | 43 | 753 | C | T | [symbol] | CDZ96151 |
| X₇₃₆₇ | M8 | 43 | 757 | G | A | [symbol] | CDZ96151 |
| X₇₃₆₈ | M8 | 43 | 1527 | C | T | [symbol] | CDZ96151 |
| X₇₃₆₉ | M8 | 43 | 1748 | G | A | [symbol] | CDZ96151 |
| X₇₃₇₀ | M8 | 43 | 1928 | C | T | [symbol] | CDZ96151 |
| X₇₃₇₁ | M8 | 62 | 26 | T | A | [symbol] | CDZ96152 |
| X₇₃₇₂ | M8 | 62 | 27 | T | C | [symbol] | CDZ96152 |
| X₇₃₇₃ | M8 | 62 | 55 | T | A | [symbol] | CDZ96152 |
| X₇₃₇₄ | M8 | 62 | 403 | C | T | [symbol] | CDZ96152 |
| X₇₃₇₅ | M8 | 62 | 875 | G | A | [symbol] | CDZ96152 |
| X₇₃₇₆ | M8 | 102 | 339 | C | T | [symbol] | CDZ96153 |
| X₇₃₇₇ | M8 | 102 | 951 | G | A | [symbol] | CDZ96153 |
| X₇₃₇₈ | M8 | 102 | 1042 | G | A | [symbol] | CDZ96153 |
| X₇₃₇₉ | M8 | 102 | 2334 | G | A | [symbol] | CDZ96153 |
| X₇₃₈₀ | M8 | 102 | 2590 | C | T | [symbol] | CDZ96153 |
| X₇₃₈₁ | M8 | 102 | 3427 | C | T | [symbol] | CDZ96153 |
| X₇₃₈₂ | M8 | 176 | 358 | G | A | [symbol] | CED80058 |
| X₇₃₈₃ | M8 | 176 | 1872 | G | A | [symbol] | CED80058 |
| X₇₃₈₄ | M8 | 176 | 1980 | G | A | [symbol] | CED80058 |
| X₇₃₈₅ | M8 | 179 | 1525 | G | A | [symbol] | CDZ96154 |
| X₇₃₈₆ | M8 | 179 | 1780 | C | T | [symbol] | CDZ96154 |
| X₇₃₈₇ | M8 | 179 | 2472 | G | A | [symbol] | CDZ96154 |
| X₇₃₈₈ | M8 | 179 | 2476 | C | T | [symbol] | CDZ96154 |
| X₇₃₈₉ | M8 | 188 | 815 | C | T | [symbol] | CED80060 |
| X₇₃₉₀ | M8 | 188 | 1174 | C | T | [symbol] | CED80060 |
| X₇₃₉₁ | M8 | 188 | 2163 | G | A | [symbol] | CED80060 |
| X₇₃₉₂ | M8 | 188 | 2170 | G | A | [symbol] | CED80060 |
| X₇₃₉₃ | M8 | 247 | 102 | G | A | [symbol] | CED80061 |
| X₇₃₉₄ | M8 | 247 | 989 | C | T | [symbol] | CED80061 |
| X₇₃₉₅ | M8 | 247 | 1053 | G | A | [symbol] | CED80061 |
| X₇₃₉₆ | M8 | 3 | 142 | C | T | [symbol] | |
| X₇₃₉₇ | M8 | 7 | 160 | T | C | [symbol] | |
| X₇₃₉₈ | M8 | 8 | 1110 | C | T | [symbol] | |
| X₇₃₉₉ | M8 | 11 | 154 | A | C | [symbol] | |
| X₇₄₀₀ | M8 | 11 | 158 | C | T | [symbol] | |
| X₇₄₀₁ | M8 | 15 | 28 | C | G | [symbol] | |
| X₇₄₀₂ | M8 | 15 | 214 | G | C | [symbol] | |
| X₇₄₀₃ | M8 | 15 | 419 | A | G | [symbol] | |
| X₇₄₀₄ | M8 | 15 | 421 | G | T | [symbol] | |
| X₇₄₀₅ | M8 | 15 | 422 | G | T | [symbol] | |
| X₇₄₀₆ | M8 | 15 | 424 | G | C | [symbol] | |
| X₇₄₀₇ | M8 | 15 | 431 | G | C | [symbol] | |
| X₇₄₀₈ | M8 | 15 | 433 | C | A | [symbol] | |
| X₇₄₀₉ | M8 | 15 | 434 | C | A | [symbol] | |
| X₇₄₁₀ | M8 | 15 | 462 | C | T | [symbol] | |
| X₇₄₁₁ | M8 | 15 | 464 | C | A | [symbol] | |
| X₇₄₁₂ | M8 | 15 | 465 | C | T | [symbol] | |
| X₇₄₁₃ | M8 | 24 | 513 | T | C | [symbol] | CDZ96160 |
| X₇₄₁₄ | M8 | 24 | 520 | C | G | [symbol] | CDZ96160 |
| X₇₄₁₅ | M8 | 24 | 5850 | C | T | [symbol] | CDZ96160 |
| X₇₄₁₆ | M8 | 24 | 118860 | C | T | [symbol] | CDZ96200 |
| X₇₄₁₇ | M8 | 24 | 180340 | G | A | [symbol] | CDZ96216 |
| X₇₄₁₈ | M8 | 24 | 266697 | G | A | [symbol] | CDZ96242 |
| X₇₄₁₉ | M8 | 24 | 307114 | G | A | [symbol] | CDZ96255 |
| X₇₄₂₀ | M8 | 24 | 335171 | C | T | [symbol] | CDZ96265 |
| X₇₄₂₁ | M8 | 24 | 340663 | T | C | [symbol] | CDZ96265 |
| X₇₄₂₂ | M8 | 24 | 349340 | C | T | [symbol] | CDZ96270 |
| X₇₄₂₃ | M8 | 24 | 349352 | G | A | [symbol] | CDZ96270 |
| X₇₄₂₄ | M8 | 24 | 363770 | C | T | [symbol] | CDZ96275 |
| X₇₄₂₅ | M8 | 24 | 420108 | G | A | [symbol] | CDZ96291 |
| X₇₄₂₆ | M8 | 24 | 422035 | G | A | [symbol] | CDZ96291 |
| X₇₄₂₇ | M8 | 24 | 484490 | C | T | [symbol] | CDZ96313 |
| X₇₄₂₈ | M8 | 27 | 445 | C | T | [symbol] | |
| X₇₄₂₉ | M8 | 27 | 1012 | A | C | [symbol] | |
| X₇₄₃₀ | M8 | 27 | 1013 | T | A | [symbol] | |
| X₇₄₃₁ | M8 | 32 | 158 | C | T | [symbol] | CDZ96332 |
| X₇₄₃₂ | M8 | 32 | 215 | C | T | [symbol] | CDZ96332 |
| X₇₄₃₃ | M8 | 32 | 2438 | C | T | [symbol] | CDZ96332 |
| X₇₄₃₄ | M8 | 32 | 2734 | C | T | [symbol] | CDZ96332 |
| X₇₄₃₅ | M8 | 32 | 2821 | C | T | [symbol] | CDZ96332 |
| X₇₄₃₆ | M8 | 32 | 3067 | C | T | [symbol] | CDZ96332 |
| X₇₄₃₇ | M8 | 32 | 3506 | C | T | [symbol] | CDZ96332 |
| X₇₄₃₈ | M8 | 32 | 3562 | G | A | [symbol] | CDZ96332 |
| X₇₄₃₉ | M8 | 33 | 399 | T | A | [symbol] | CED82002 |
| X₇₄₄₀ | M8 | 33 | 24784 | C | T | [symbol] | CED82008 |
| X₇₄₄₁ | M8 | 33 | 45488 | C | T | [symbol] | CED82012 |
| X₇₄₄₂ | M8 | 33 | 64676 | G | A | [symbol] | CED82019 |
| X₇₄₄₃ | M8 | 33 | 79913 | G | A | [symbol] | CED82024 |
| X₇₄₄₄ | M8 | 33 | 109397 | G | A | [symbol] | CED82038 |
| X₇₄₄₅ | M8 | 33 | 182417 | G | A | [symbol] | CED82062 |
| X₇₄₄₆ | M8 | 33 | 198050 | C | T | [symbol] | CED82067 |
| X₇₄₄₇ | M8 | 33 | 257786 | C | T | [symbol] | CED82084 |
| X₇₄₄₈ | M8 | 33 | 260027 | G | A | [symbol] | CED82084 |
| X₇₄₄₉ | M8 | 33 | 274627 | G | A | [symbol] | CED82091 |
| X₇₄₅₀ | M8 | 33 | 276128 | C | T | [symbol] | CED82092 |
| X₇₄₅₁ | M8 | 33 | 283808 | C | T | [symbol] | CED82093 |
| X₇₄₅₂ | M8 | 33 | 318242 | G | A | [symbol] | CED82105 |
| X₇₄₅₃ | M8 | 33 | 326016 | T | C | [symbol] | CED82108 |
| X₇₄₅₄ | M8 | 33 | 327840 | C | T | [symbol] | CED82108 |
| X₇₄₅₅ | M8 | 33 | 341890 | G | A | [symbol] | CED82113 |
| X₇₄₅₆ | M8 | 33 | 374131 | A | G | [symbol] | CED82121- |
| | | | | | | | CED82122 |
| X₇₄₅₇ | M8 | 33 | 374134 | A | G | [symbol] | CED82121- |
| | | | | | | | CED82122 |
| X₇₄₅₈ | M8 | 33 | 374137 | A | T | [symbol] | CED82121- |
| | | | | | | | CED82122 |
| X₇₄₅₉ | M8 | 33 | 374150 | A | C | [symbol] | CED82121- |
| | | | | | | | CED82122 |
| X₇₄₆₀ | M8 | 33 | 375339 | A | G | [symbol] | CED82121- |
| | | | | | | | CED82122 |
| X₇₄₆₁ | M8 | 33 | 375366 | T | C | [symbol] | CED82121- |
| | | | | | | | CED82122 |
| X₇₄₆₂ | M8 | 33 | 385139 | G | C | [symbol] | CED82122 |
| X₇₄₆₃ | M8 | 33 | 385155 | T | C | [symbol] | CED82122 |
| X₇₄₆₄ | M8 | 33 | 385319 | A | C | [symbol] | CED82122 |
| X₇₄₆₅ | M8 | 33 | 385527 | C | T | [symbol] | CED82122 |
| X₇₄₆₆ | M8 | 33 | 386116 | G | C | [symbol] | CED82124 |
| X₇₄₆₇ | M8 | 33 | 428329 | G | A | [symbol] | CED82136 |
| X₇₄₆₈ | M8 | 33 | 448815 | G | A | [symbol] | CED82144 |
| X₇₄₆₉ | M8 | 33 | 453975 | C | T | [symbol] | CED82147 |
| X₇₄₇₀ | M8 | 33 | 455451 | C | T | [symbol] | CED82146 |
| X₇₄₇₁ | M8 | 33 | 476006 | A | G | [symbol] | CED82153 |
| X₇₄₇₂ | M8 | 33 | 481361 | G | A | [symbol] | CED82157 |
| X₇₄₇₃ | M8 | 33 | 509127 | C | T | [symbol] | CED82166 |
| X₇₄₇₄ | M8 | 33 | 628247 | C | T | [symbol] | CED82205 |
| X₇₄₇₅ | M8 | 33 | 639242 | G | A | [symbol] | CED82208 |
| X₇₄₇₆ | M8 | 33 | 652012 | G | A | [symbol] | CED82212 |
| X₇₄₇₇ | M8 | 33 | 699423 | C | T | [symbol] | CED82227 |
| X₇₄₇₈ | M8 | 33 | 715901 | C | T | [symbol] | CED82231 |
| X₇₄₇₉ | M8 | 33 | 729705 | C | T | [symbol] | CED82234 |
| X₇₄₈₀ | M8 | 33 | 750883 | T | C | [symbol] | CED82240 |
| X₇₄₈₁ | M8 | 33 | 816048 | C | T | [symbol] | CED82262 |
| X₇₄₈₂ | M8 | 33 | 822184 | G | A | [symbol] | CED82264 |
| X₇₄₈₃ | M8 | 33 | 829825 | C | T | [symbol] | CED82271 |
| X₇₄₈₄ | M8 | 33 | 917771 | A | T | [symbol] | CED82294 |
| X₇₄₈₅ | M8 | 33 | 936571 | A | G | [symbol] | CED82300 |
| X₇₄₈₆ | M8 | 33 | 936600 | G | A | [symbol] | CED82300 |
| X₇₄₈₇ | M8 | 33 | 936667 | A | G | [symbol] | CED82300 |
| X₇₄₈₈ | M8 | 33 | 938634 | A | G | [symbol] | CED82300 |
| X₇₄₈₉ | M8 | 33 | 947993 | T | A | [symbol] | CED82302 |
| X₇₄₉₀ | M8 | 33 | 972701 | C | T | [symbol] | CED82309 |
| X₇₄₉₁ | M8 | 33 | 1049699 | G | A | [symbol] | CED82334 |
| X₇₄₉₂ | M8 | 33 | 1087834 | G | A | [symbol] | CED82350 |
| X₇₄₉₃ | M8 | 33 | 1088320 | C | T | [symbol] | CED82349 |
| X₇₄₉₄ | M8 | 33 | 1127676 | G | A | [symbol] | CED82358 |
| X₇₄₉₅ | M8 | 33 | 1138444 | C | T | [symbol] | CED82363 |
| X₇₄₉₆ | M8 | 33 | 1165914 | G | A | [symbol] | CED82372 |
| X₇₄₉₇ | M8 | 33 | 1191727 | C | T | [symbol] | CED82379 |
| X₇₄₉₈ | M8 | 33 | 1311181 | G | A | [symbol] | CED82413 |
| X₇₄₉₉ | M8 | 33 | 1316789 | C | T | [symbol] | CED82415 |
| X₇₅₀₀ | M8 | 33 | 1324395 | G | A | [symbol] | CED82417 |
| X₇₅₀₁ | M8 | 33 | 1339376 | C | T | [symbol] | CED82424 |
| X₇₅₀₂ | M8 | 33 | 1349009 | C | T | [symbol] | CED82427 |
| X₇₅₀₃ | M8 | 33 | 1367015 | C | T | [symbol] | CED82432 |
| X₇₅₀₄ | M8 | 33 | 1411059 | C | T | [symbol] | CED82446 |
| X₇₅₀₅ | M8 | 33 | 1460436 | C | T | [symbol] | CED82462 |
| X₇₅₀₆ | M8 | 33 | 1499347 | G | A | [symbol] | CED82472 |
| X₇₅₀₇ | M8 | 33 | 1507482 | A | G | [symbol] | CED82477 |
| X₇₅₀₈ | M8 | 33 | 1514686 | C | T | [symbol] | CED82478 |
| X₇₅₀₉ | M8 | 33 | 1515148 | G | A | [symbol] | CED82478 |
| X₇₅₁₀ | M8 | 33 | 1537835 | G | A | [symbol] | CED82483 |
| X₇₅₁₁ | M8 | 33 | 1544767 | A | C | [symbol] | CED82485- |
| | | | | | | | CED82486 |
| X₇₅₁₂ | M8 | 33 | 1585699 | C | T | [symbol] | CED82497 |
| X₇₅₁₃ | M8 | 33 | 1587443 | C | T | [symbol] | CED82497 |
| X₇₅₁₄ | M8 | 33 | 1596242 | C | T | [symbol] | CED82498 |
| X₇₅₁₅ | M8 | 33 | 1638726 | G | A | [symbol] | CED82513 |
| X₇₅₁₆ | M8 | 33 | 1642659 | G | A | [symbol] | CED82515 |
| X₇₅₁₇ | M8 | 33 | 1651969 | A | T | [symbol] | CED82517 |
| X₇₅₁₈ | M8 | 33 | 1689192 | C | T | [symbol] | CED82529 |
| X₇₅₁₉ | M8 | 33 | 1700000 | G | A | [symbol] | CED82534 |
| X₇₅₂₀ | M8 | 33 | 1716261 | C | T | [symbol] | CED82538 |
| X₇₅₂₁ | M8 | 33 | 1728730 | G | A | [symbol] | CED82541 |
| X₇₅₂₂ | M8 | 33 | 1740595 | C | T | [symbol] | CED82545 |
| X₇₅₂₃ | M8 | 33 | 1745517 | C | T | [symbol] | CED82546 |
| X₇₅₂₄ | M8 | 33 | 1780044 | G | A | [symbol] | CED82555 |
| X₇₅₂₅ | M8 | 33 | 1838447 | G | A | [symbol] | CED82575 |
| X₇₅₂₆ | M8 | 33 | 1841263 | G | A | [symbol] | CED82576 |
| X₇₅₂₇ | M8 | 33 | 1848935 | C | T | [symbol] | CED82579 |
| X₇₅₂₈ | M8 | 33 | 1860991 | G | A | [symbol] | CED82583 |
| X₇₅₂₉ | M8 | 33 | 1887401 | C | T | [symbol] | CED82591 |
| X₇₅₃₀ | M8 | 33 | 1905896 | G | A | [symbol] | CED82599 |
| X₇₅₃₁ | M8 | 33 | 1959142 | G | A | [symbol] | CED82619 |
| X₇₅₃₂ | M8 | 33 | 1967112 | C | T | [symbol] | CED82622 |
| X₇₅₃₃ | M8 | 33 | 2010230 | C | T | [symbol] | CED82636 |
| X₇₅₃₄ | M8 | 33 | 2034962 | G | A | [symbol] | CED82645 |
| X₇₅₃₅ | M8 | 33 | 2046185 | C | T | [symbol] | CED82650 |
| X₇₅₃₆ | M8 | 36 | 320 | G | A | [symbol] | |
| X₇₅₃₇ | M8 | 36 | 1056 | G | A | [symbol] | |
| X₇₅₃₈ | M8 | 36 | 1295 | C | T | [symbol] | |
| X₇₅₃₉ | M8 | 36 | 1461 | A | T | [symbol] | |
| X₇₅₄₀ | M8 | 42 | 804 | C | T | [symbol] | CDZ96333 |
| X₇₅₄₁ | M8 | 42 | 1559 | G | A | [symbol] | CDZ96333 |
| X₇₅₄₂ | M8 | 48 | 563 | C | T | [symbol] | |
| X₇₅₄₃ | M8 | 48 | 565 | T | C | [symbol] | |
| X₇₅₄₄ | M8 | 48 | 576 | G | A | [symbol] | |
| X₇₅₄₅ | M8 | 48 | 580 | T | A | [symbol] | |
| X₇₅₄₆ | M8 | 48 | 720 | A | G | [symbol] | |
| X₇₅₄₇ | M8 | 50 | 145 | G | A | [symbol] | |
| X₇₅₄₈ | M8 | 52 | 123705 | C | T | [symbol] | CED82700 |
| X₇₅₄₉ | M8 | 52 | 144382 | C | T | [symbol] | CED82709 |
| X₇₅₅₀ | M8 | 52 | 145060 | G | A | [symbol] | CED82708 |
| X₇₅₅₁ | M8 | 52 | 151974 | C | T | [symbol] | CED82712 |
| X₇₅₅₂ | M8 | 52 | 183986 | C | T | [symbol] | CED82722 |
| X₇₅₅₃ | M8 | 52 | 207354 | C | T | [symbol] | CED82730 |
| X₇₅₅₄ | M8 | 52 | 240241 | T | C | [symbol] | CED82734 |
| X₇₅₅₅ | M8 | 52 | 243203 | G | A | [symbol] | CED82733 |
| X₇₅₅₆ | M8 | 52 | 244305 | G | A | [symbol] | CED82734 |
| X₇₅₅₇ | M8 | 52 | 284733 | G | A | [symbol] | CED82747 |
| X₇₅₅₈ | M8 | 52 | 344346 | G | A | [symbol] | CED82767 |
| X₇₅₅₉ | M8 | 52 | 358601 | G | A | [symbol] | CED82772 |
| X₇₅₆₀ | M8 | 52 | 360279 | C | T | [symbol] | CED82772 |
| X₇₅₆₁ | M8 | 52 | 390095 | G | A | [symbol] | CED82784 |
| X₇₅₆₂ | M8 | 52 | 390988 | C | T | [symbol] | CED82785 |
| X₇₅₆₃ | M8 | 52 | 395374 | G | A | [symbol] | CED82786 |
| X₇₅₆₄ | M8 | 52 | 398723 | C | T | [symbol] | CED82786 |
| X₇₅₆₅ | M8 | 52 | 398728 | C | T | [symbol] | CED82786 |
| X₇₅₆₆ | M8 | 52 | 473123 | G | A | [symbol] | CED82810 |
| X₇₅₆₇ | M8 | 52 | 487472 | C | T | [symbol] | CED82814 |
| X₇₅₆₈ | M8 | 52 | 516267 | C | T | [symbol] | CED82823 |
| X₇₅₆₉ | M8 | 52 | 542542 | C | T | [symbol] | CED82833 |
| X₇₅₇₀ | M8 | 52 | 552099 | G | A | [symbol] | CED82837 |
| X₇₅₇₁ | M8 | 52 | 552173 | G | A | [symbol] | CED82837 |
| X₇₅₇₂ | M8 | 52 | 553027 | G | A | [symbol] | CED82837 |
| X₇₅₇₃ | M8 | 52 | 557092 | C | T | [symbol] | CED82840 |
| X₇₅₇₄ | M8 | 52 | 631479 | C | T | [symbol] | CED82860 |
| X₇₅₇₅ | M8 | 52 | 697002 | C | T | [symbol] | CED82888 |
| X₇₅₇₆ | M8 | 52 | 700204 | G | A | [symbol] | CED82888 |
| X₇₅₇₇ | M8 | 52 | 710492 | C | T | [symbol] | CED82891 |
| X₇₅₇₈ | M8 | 52 | 725138 | C | T | [symbol] | CED82899 |
| X₇₅₇₉ | M8 | 52 | 741861 | G | A | [symbol] | CED82905 |
| X₇₅₈₀ | M8 | 52 | 746548 | G | A | [symbol] | CED82907 |
| X₇₅₈₁ | M8 | 52 | 783981 | C | T | [symbol] | CED82919 |
| X₇₅₈₂ | M8 | 52 | 817452 | T | A | [symbol] | CED82928 |
| X₇₅₈₃ | M8 | 52 | 819924 | C | T | [symbol] | CED82929 |
| X₇₅₈₄ | M8 | 52 | 840755 | C | T | [symbol] | CED82935 |
| X₇₅₈₅ | M8 | 52 | 849671 | G | A | [symbol] | CED82940 |
| X₇₅₈₆ | M8 | 52 | 928854 | C | T | [symbol] | CED82971 |
| X₇₅₈₇ | M8 | 52 | 938650 | G | A | [symbol] | CED82973 |
| X₇₅₈₈ | M8 | 52 | 967316 | A | T | [symbol] | CED82984 |
| X₇₅₈₉ | M8 | 52 | 967701 | T | A | [symbol] | CED82983 |
| X₇₅₉₀ | M8 | 52 | 1003899 | G | A | [symbol] | CED83001 |
| X₇₅₉₁ | M8 | 52 | 1009554 | C | T | [symbol] | CED83003 |
| X₇₅₉₂ | M8 | 52 | 1010007 | C | T | [symbol] | CED83002 |
| X₇₅₉₃ | M8 | 52 | 1020804 | G | A | [symbol] | CED83005 |
| X₇₅₉₄ | M8 | 52 | 1021202 | C | T | [symbol] | CED83005 |
| X₇₅₉₅ | M8 | 52 | 1029498 | G | A | [symbol] | CED83007 |
| X₇₅₉₆ | M8 | 52 | 1043011 | C | T | [symbol] | CED83012 |
| X₇₅₉₇ | M8 | 52 | 1067895 | C | T | [symbol] | CED83020 |
| X₇₅₉₈ | M8 | 52 | 1158103 | C | T | [symbol] | CED83051 |
| X₇₅₉₉ | M8 | 52 | 1163067 | G | A | [symbol] | CED83051 |
| X₇₆₀₀ | M8 | 52 | 1227910 | G | A | [symbol] | CED83074 |
| X₇₆₀₁ | M8 | 52 | 1297876 | G | A | [symbol] | CED83099 |
| X₇₆₀₂ | M8 | 52 | 1336560 | G | A | [symbol] | CED83113 |
| X₇₆₀₃ | M8 | 52 | 1350463 | C | T | [symbol] | CED83118 |
| X₇₆₀₄ | M8 | 52 | 1356590 | C | T | [symbol] | CED83120 |
| X₇₆₀₅ | M8 | 52 | 1362217 | C | T | [symbol] | CED83122 |
| X₇₆₀₆ | M8 | 52 | 1363357 | G | A | [symbol] | CED83122 |
| X₇₆₀₇ | M8 | 52 | 1363405 | C | T | [symbol] | CED83123 |
| X₇₆₀₈ | M8 | 52 | 1365822 | C | T | [symbol] | CED83124 |
| X₇₆₀₉ | M8 | 52 | 1392238 | C | T | [symbol] | CED83130 |
| X₇₆₁₀ | M8 | 52 | 1399439 | G | A | [symbol] | CED83134 |
| X₇₆₁₁ | M8 | 52 | 1458571 | C | T | [symbol] | CED83158 |
| X₇₆₁₂ | M8 | 52 | 1475408 | C | T | [symbol] | CED83166 |
| X₇₆₁₃ | M8 | 52 | 1525824 | G | T | [symbol] | CED83180 |
| X₇₆₁₄ | M8 | 52 | 1526796 | G | A | [symbol] | CED83181 |
| X₇₆₁₅ | M8 | 52 | 1540879 | C | T | [symbol] | CED83186 |
| X₇₆₁₆ | M8 | 52 | 1583655 | C | T | [symbol] | CED83206 |
| X₇₆₁₇ | M8 | 52 | 1621166 | C | T | [symbol] | CED83219 |
| X₇₆₁₈ | M8 | 52 | 1700952 | G | A | [symbol] | CED83246 |
| X₇₆₁₉ | M8 | 52 | 1708900 | A | G | [symbol] | CED83249 |
| X₇₆₂₀ | M8 | 52 | 1715136 | C | T | [symbol] | CED83250 |
| X₇₆₂₁ | M8 | 52 | 1715899 | C | T | [symbol] | CED83250 |
| X₇₆₂₂ | M8 | 52 | 1716210 | C | T | [symbol] | CED83250 |
| X₇₆₂₃ | M8 | 52 | 1760440 | G | A | [symbol] | CED83266 |
| X₇₆₂₄ | M8 | 52 | 1765533 | G | A | [symbol] | CED83268 |
| X₇₆₂₅ | M8 | 52 | 1787135 | C | T | [symbol] | CED83275 |
| X₇₆₂₆ | M8 | 52 | 1793370 | G | A | [symbol] | CED83275 |
| X₇₆₂₇ | M8 | 52 | 1796780 | G | A | [symbol] | CED83279 |
| X₇₆₂₈ | M8 | 52 | 1807309 | G | A | [symbol] | CED83283 |
| X₇₆₂₉ | M8 | 52 | 1864608 | G | A | [symbol] | CED83303 |
| X₇₆₃₀ | M8 | 52 | 1871310 | C | T | [symbol] | CED83304 |
| X₇₆₃₁ | M8 | 52 | 1874150 | G | T | [symbol] | CED83305 |
| X₇₆₃₂ | M8 | 52 | 1874531 | C | T | [symbol] | CED83306 |
| X₇₆₃₃ | M8 | 52 | 1888989 | C | T | [symbol] | CED83312 |
| X₇₆₃₄ | M8 | 52 | 1892615 | C | T | [symbol] | CED83313 |
| X₇₆₃₅ | M8 | 52 | 1915927 | G | A | [symbol] | CED83315 |
| X₇₆₃₆ | M8 | 52 | 1938393 | C | T | [symbol] | CED83322 |
| X₇₆₃₇ | M8 | 52 | 1956479 | C | T | [symbol] | CED83330 |
| X₇₆₃₈ | M8 | 52 | 1987096 | G | A | [symbol] | CED83341 |
| X₇₆₃₉ | M8 | 52 | 2041781 | C | T | [symbol] | CED83355 |
| X₇₆₄₀ | M8 | 52 | 2047954 | G | A | [symbol] | CED83358 |
| X₇₆₄₁ | M8 | 52 | 2050245 | G | A | [symbol] | CED83359 |
| X₇₆₄₂ | M8 | 52 | 2071127 | C | T | [symbol] | CED83365 |
| X₇₆₄₃ | M8 | 52 | 2081390 | G | A | [symbol] | CED83367 |
| X₇₆₄₄ | M8 | 52 | 2093148 | C | T | [symbol] | CED83368 |
| X₇₆₄₅ | M8 | 52 | 2093251 | C | T | [symbol] | CED83368 |
| X₇₆₄₆ | M8 | 52 | 2120665 | C | T | [symbol] | CED83376 |
| X₇₆₄₇ | M8 | 52 | 2136885 | A | G | [symbol] | CED83382 |
| X₇₆₄₈ | M8 | 52 | 2162878 | G | A | [symbol] | CED83392 |
| X₇₆₄₉ | M8 | 52 | 2171271 | C | T | [symbol] | CED83395 |
| X₇₆₅₀ | M8 | 52 | 2258192 | C | T | [symbol] | CED83422 |
| X₇₆₅₁ | M8 | 52 | 2265219 | C | T | [symbol] | CED83425 |
| X₇₆₅₂ | M8 | 52 | 2266378 | C | T | [symbol] | CED83423 |
| X₇₆₅₃ | M8 | 52 | 2296638 | G | A | [symbol] | CED83432 |
| X₇₆₅₄ | M8 | 52 | 2308421 | T | C | [symbol] | CED83439 |
| X₇₆₅₅ | M8 | 52 | 2327266 | C | T | [symbol] | CED83445 |
| X₇₆₅₆ | M8 | 52 | 2400778 | C | T | [symbol] | CED83463 |
| X₇₆₅₇ | M8 | 52 | 2531584 | C | T | [symbol] | CED83507 |
| X₇₆₅₈ | M8 | 52 | 2543220 | C | T | [symbol] | CED83509 |
| X₇₆₅₉ | M8 | 52 | 2549953 | A | G | [symbol] | CED83516 |
| X₇₆₆₀ | M8 | 52 | 2566106 | C | T | [symbol] | CED83520 |
| X₇₆₆₁ | M8 | 52 | 2575090 | A | G | [symbol] | CED83525 |
| X₇₆₆₂ | M8 | 53 | 26291 | G | A | [symbol] | CDZ96348 |
| X₇₆₆₃ | M8 | 54 | 4815 | G | A | [symbol] | CDZ96384 |
| X₇₆₆₄ | M8 | 54 | 4816 | A | T | [symbol] | CDZ96384 |
| X₇₆₆₅ | M8 | 54 | 4817 | G | C | [symbol] | CDZ96384 |
| X₇₆₆₆ | M8 | 54 | 4821 | G | A | [symbol] | CDZ96384 |
| X₇₆₆₇ | M8 | 54 | 16963 | C | T | [symbol] | CDZ96386 |
| X₇₆₆₈ | M8 | 54 | 72235 | G | A | [symbol] | CDZ96406 |
| X₇₆₆₉ | M8 | 54 | 106086 | G | A | [symbol] | CDZ96416 |
| X₇₆₇₀ | M8 | 54 | 119439 | C | T | [symbol] | CDZ96420 |
| X₇₆₇₁ | M8 | 54 | 129380 | C | T | [symbol] | CDZ96423 |
| X₇₆₇₂ | M8 | 54 | 164317 | A | T | [symbol] | CDZ96437 |
| X₇₆₇₃ | M8 | 54 | 194230 | G | A | [symbol] | CDZ96446 |
| X₇₆₇₄ | M8 | 54 | 195804 | G | A | [symbol] | CDZ96447 |
| X₇₆₇₅ | M8 | 54 | 202467 | C | T | [symbol] | CDZ96448 |
| X₇₆₇₆ | M8 | 54 | 206494 | G | A | [symbol] | CDZ96450 |
| X₇₆₇₇ | M8 | 54 | 207267 | G | A | [symbol] | CDZ96450 |
| X₇₆₇₈ | M8 | 54 | 207737 | C | T | [symbol] | CDZ96450 |
| X₇₆₇₉ | M8 | 54 | 245840 | G | A | [symbol] | CDZ96464 |
| X₇₆₈₀ | M8 | 54 | 274617 | A | T | [symbol] | CDZ96471 |
| X₇₆₈₁ | M8 | 54 | 278479 | C | T | [symbol] | CDZ96474 |
| X₇₆₈₂ | M8 | 54 | 303553 | T | C | [symbol] | CDZ96484 |
| X₇₆₈₃ | M8 | 54 | 357141 | G | A | [symbol] | CDZ96496 |
| X₇₆₈₄ | M8 | 54 | 370320 | G | A | [symbol] | CDZ96499 |
| X₇₆₈₅ | M8 | 54 | 391223 | A | G | [symbol] | CDZ96509 |
| X₇₆₈₆ | M8 | 54 | 395454 | G | A | [symbol] | CDZ96509 |
| X₇₆₈₇ | M8 | 54 | 413765 | G | A | [symbol] | CDZ96516 |
| X₇₆₈₈ | M8 | 54 | 427542 | C | T | [symbol] | CDZ96520 |
| X₇₆₈₉ | M8 | 54 | 431404 | C | T | [symbol] | CDZ96520 |
| X₇₆₉₀ | M8 | 54 | 478527 | C | T | [symbol] | CDZ96537 |
| X₇₆₉₁ | M8 | 54 | 537282 | T | C | [symbol] | CDZ96557 |
| X₇₆₉₂ | M8 | 54 | 583996 | G | A | [symbol] | CDZ96570 |
| X₇₆₉₃ | M8 | 54 | 616268 | A | G | [symbol] | CDZ96581 |
| X₇₆₉₄ | M8 | 54 | 631489 | G | A | [symbol] | CDZ96585 |
| X₇₆₉₅ | M8 | 54 | 733193 | T | C | [symbol] | CDZ96619 |
| X₇₆₉₆ | M8 | 54 | 743396 | G | A | [symbol] | CDZ96621 |
| X₇₆₉₇ | M8 | 58 | 93 | T | C | [symbol] | |
| X₇₆₉₈ | M8 | 64 | 2113 | G | A | [symbol] | CDZ96626 |
| X₇₆₉₉ | M8 | 65 | 124 | C | T | [symbol] | CDZ96627 |
| X₇₇₀₀ | M8 | 67 | 111 | G | A | [symbol] | |
| X₇₇₀₁ | M8 | 67 | 176 | C | T | [symbol] | |
| X₇₇₀₂ | M8 | 69 | 19892 | T | C | [symbol] | CED83533 |
| X₇₇₀₃ | M8 | 69 | 19893 | G | A | [symbol] | CED83533 |
| X₇₇₀₄ | M8 | 69 | 20845 | C | T | [symbol] | CED83532 |
| X₇₇₀₅ | M8 | 69 | 29006 | G | A | [symbol] | CED83534 |
| X₇₇₀₆ | M8 | 69 | 48869 | G | A | [symbol] | CED83542 |
| X₇₇₀₇ | M8 | 69 | 52428 | G | A | [symbol] | CED83541 |
| X₇₇₀₈ | M8 | 69 | 55826 | G | A | [symbol] | CED83545 |
| X₇₇₀₉ | M8 | 69 | 82710 | G | A | [symbol] | CED83554 |
| X₇₇₁₀ | M8 | 69 | 107920 | C | T | [symbol] | CED83561 |
| X₇₇₁₁ | M8 | 69 | 148757 | C | T | [symbol] | CED83575 |
| X₇₇₁₂ | M8 | 69 | 179873 | C | T | [symbol] | CED83585 |
| X₇₇₁₃ | M8 | 69 | 196810 | G | A | [symbol] | CED83594 |
| X₇₇₁₄ | M8 | 69 | 201005 | G | A | [symbol] | CED83595 |
| X₇₇₁₅ | M8 | 69 | 201292 | G | A | [symbol] | CED83595 |
| X₇₇₁₆ | M8 | 69 | 312480 | A | G | [symbol] | CED83633 |
| X₇₇₁₇ | M8 | 69 | 371916 | C | T | [symbol] | CED83653 |
| X₇₇₁₈ | M8 | 69 | 384418 | G | A | [symbol] | CED83656 |
| X₇₇₁₉ | M8 | 69 | 386371 | C | T | [symbol] | CED83657 |
| X₇₇₂₀ | M8 | 69 | 415659 | C | T | [symbol] | CED83665 |
| X₇₇₂₁ | M8 | 69 | 431248 | G | A | [symbol] | CED83671 |
| X₇₇₂₂ | M8 | 69 | 461986 | G | A | [symbol] | CED83680 |
| X₇₇₂₃ | M8 | 69 | 523701 | C | T | [symbol] | CED83701 |
| X₇₇₂₄ | M8 | 69 | 534495 | C | T | [symbol] | CED83704 |
| X₇₇₂₅ | M8 | 69 | 585845 | G | A | [symbol] | CED83722 |
| X₇₇₂₆ | M8 | 69 | 631511 | G | A | [symbol] | CED83742 |
| X₇₇₂₇ | M8 | 69 | 637299 | T | C | [symbol] | CED83744 |
| X₇₇₂₈ | M8 | 69 | 638135 | G | A | [symbol] | CED83744 |
| X₇₇₂₉ | M8 | 69 | 675579 | G | A | [symbol] | CED83760 |
| X₇₇₃₀ | M8 | 69 | 676581 | G | A | [symbol] | CED83760 |
| X₇₇₃₁ | M8 | 69 | 676853 | G | A | [symbol] | CED83759 |
| X₇₇₃₂ | M8 | 69 | 678406 | C | T | [symbol] | CED83759 |
| X₇₇₃₃ | M8 | 69 | 682770 | C | T | [symbol] | CED83763 |
| X₇₇₃₄ | M8 | 69 | 741255 | C | T | [symbol] | CED83781 |
| X₇₇₃₅ | M8 | 69 | 761819 | C | T | [symbol] | CED83789 |
| X₇₇₃₆ | M8 | 69 | 777063 | G | A | [symbol] | CED83792 |
| X₇₇₃₇ | M8 | 69 | 859081 | C | T | [symbol] | CED83813 |
| X₇₇₃₈ | M8 | 69 | 865820 | C | T | [symbol] | CED83814 |
| X₇₇₃₉ | M8 | 69 | 869706 | G | A | [symbol] | CED83815 |
| X₇₇₄₀ | M8 | 69 | 892199 | G | A | [symbol] | CED83822 |
| X₇₇₄₁ | M8 | 69 | 894019 | G | A | [symbol] | CED83821 |
| X₇₇₄₂ | M8 | 69 | 894462 | C | T | [symbol] | CED83821 |
| X₇₇₄₃ | M8 | 69 | 930700 | C | T | [symbol] | CED83835 |
| X₇₇₄₄ | M8 | 69 | 971219 | C | T | [symbol] | CED83847 |
| X₇₇₄₅ | M8 | 69 | 988364 | G | A | [symbol] | CED83857 |
| X₇₇₄₆ | M8 | 69 | 989781 | G | A | [symbol] | CED83858 |
| X₇₇₄₇ | M8 | 69 | 994927 | C | T | [symbol] | CED83859 |
| X₇₇₄₈ | M8 | 69 | 1017805 | G | A | [symbol] | CED83865 |
| X₇₇₄₉ | M8 | 69 | 1038140 | G | A | [symbol] | CED83874 |
| X₇₇₅₀ | M8 | 69 | 1072756 | G | A | [symbol] | CED83886 |
| X₇₇₅₁ | M8 | 69 | 1084824 | G | A | [symbol] | CED83891 |
| X₇₇₅₂ | M8 | 69 | 1084835 | C | T | [symbol] | CED83891 |
| X₇₇₅₃ | M8 | 69 | 1084848 | A | G | [symbol] | CED83891 |
| X₇₇₅₄ | M8 | 69 | 1084866 | A | G | [symbol] | CED83891 |
| X₇₇₅₅ | M8 | 69 | 1084931 | C | T | [symbol] | CED83891 |
| X₇₇₅₆ | M8 | 69 | 1102164 | C | T | [symbol] | CED83897 |
| X₇₇₅₇ | M8 | 69 | 1109785 | C | T | [symbol] | CED83899 |
| X₇₇₅₈ | M8 | 69 | 1109813 | A | G | [symbol] | CED83899 |
| X₇₇₅₉ | M8 | 69 | 1109818 | A | G | [symbol] | CED83899 |
| X₇₇₆₀ | M8 | 69 | 1109819 | T | A | [symbol] | CED83899 |
| X₇₇₆₁ | M8 | 69 | 1109823 | T | C | [symbol] | CED83899 |
| X₇₇₆₂ | M8 | 69 | 1109830 | A | G | [symbol] | CED83899 |
| X₇₇₆₃ | M8 | 69 | 1109831 | C | A | [symbol] | CED83899 |
| X₇₇₆₄ | M8 | 69 | 1109835 | A | G | [symbol] | CED83899 |
| X₇₇₆₅ | M8 | 69 | 1143597 | C | G | [symbol] | CED83911 |
| X₇₇₆₆ | M8 | 69 | 1153118 | G | A | [symbol] | CED83915 |
| X₇₇₆₇ | M8 | 69 | 1199155 | C | T | [symbol] | CED83929 |
| X₇₇₆₈ | M8 | 69 | 1218409 | G | A | [symbol] | CED83936 |
| X₇₇₆₉ | M8 | 69 | 1228996 | G | A | [symbol] | CED83939 |
| X₇₇₇₀ | M8 | 69 | 1229023 | G | A | [symbol] | CED83939 |
| X₇₇₇₁ | M8 | 69 | 1245509 | C | T | [symbol] | CED83943 |
| X₇₇₇₂ | M8 | 69 | 1255967 | C | T | [symbol] | CED83949 |
| X₇₇₇₃ | M8 | 69 | 1260087 | G | A | [symbol] | CED83949 |
| X₇₇₇₄ | M8 | 69 | 1285491 | T | C | [symbol] | CED83961 |
| X₇₇₇₅ | M8 | 69 | 1326247 | G | A | [symbol] | CED83969 |
| X₇₇₇₆ | M8 | 69 | 1342909 | G | A | [symbol] | CED83975 |
| X₇₇₇₇ | M8 | 69 | 1372486 | C | T | [symbol] | CED83987 |
| X₇₇₇₈ | M8 | 69 | 1411237 | C | T | [symbol] | CED84001 |
| X₇₇₇₉ | M8 | 69 | 1411421 | C | T | [symbol] | CED83999 |
| X₇₇₈₀ | M8 | 69 | 1412234 | G | A | [symbol] | CED83999 |
| X₇₇₈₁ | M8 | 69 | 1433913 | C | T | [symbol] | CED84007 |
| X₇₇₈₂ | M8 | 69 | 1485040 | G | A | [symbol] | CED84023 |
| X₇₇₈₃ | M8 | 69 | 1529637 | C | T | [symbol] | CED84038 |
| X₇₇₈₄ | M8 | 69 | 1539964 | C | T | [symbol] | CED84041 |
| X₇₇₈₅ | M8 | 69 | 1590302 | G | A | [symbol] | CED84059 |
| X₇₇₈₆ | M8 | 69 | 1601173 | C | T | [symbol] | CED84062 |
| X₇₇₈₇ | M8 | 69 | 1652288 | C | T | [symbol] | CED84077 |
| X₇₇₈₈ | M8 | 69 | 1674261 | C | T | [symbol] | CED84084 |
| X₇₇₈₉ | M8 | 69 | 1678475 | C | T | [symbol] | CED84085 |
| X₇₇₉₀ | M8 | 69 | 1687066 | A | G | [symbol] | CED84088 |
| X₇₇₉₁ | M8 | 69 | 1687394 | G | A | [symbol] | CED84087 |
| X₇₇₉₂ | M8 | 69 | 1688555 | C | T | [symbol] | CED84088 |
| X₇₇₉₃ | M8 | 69 | 1688570 | C | T | [symbol] | CED84088 |
| X₇₇₉₄ | M8 | 69 | 1714728 | G | A | [symbol] | CED84097 |
| X₇₇₉₅ | M8 | 69 | 1735172 | G | A | [symbol] | CED84108 |
| X₇₇₉₆ | M8 | 69 | 1740723 | T | C | [symbol] | CED84109 |
| X₇₇₉₇ | M8 | 69 | 1771849 | C | T | [symbol] | CED84116 |
| X₇₇₉₈ | M8 | 69 | 1784836 | C | T | [symbol] | CED84123 |
| X₇₇₉₉ | M8 | 69 | 1792597 | C | T | [symbol] | CED84126 |
| X₇₈₀₀ | M8 | 69 | 1800584 | G | A | [symbol] | CED84129 |
| X₇₈₀₁ | M8 | 69 | 1854739 | G | A | [symbol] | CED84145 |
| X₇₈₀₂ | M8 | 69 | 1855951 | C | T | [symbol] | CED84145 |
| X₇₈₀₃ | M8 | 69 | 1871563 | G | A | [symbol] | CED84153 |
| X₇₈₀₄ | M8 | 69 | 1924621 | C | T | [symbol] | CED84172 |
| X₇₈₀₅ | M8 | 69 | 1967529 | C | T | [symbol] | CED84189 |
| X₇₈₀₆ | M8 | 69 | 1968848 | A | G | [symbol] | CED84189 |
| X₇₈₀₇ | M8 | 69 | 2001469 | C | T | [symbol] | CED84197 |
| X₇₈₀₈ | M8 | 69 | 2089848 | A | G | [symbol] | CED84232 |
| X₇₈₀₉ | M8 | 69 | 2089870 | C | T | [symbol] | CED84232 |
| X₇₈₁₀ | M8 | 69 | 2089876 | C | A | [symbol] | CED84232 |
| X₇₈₁₁ | M8 | 69 | 2156050 | C | T | [symbol] | CED84252 |
| X₇₈₁₂ | M8 | 69 | 2160207 | C | T | [symbol] | CED84255 |
| X₇₈₁₃ | M8 | 69 | 2213959 | C | T | [symbol] | CED84269 |
| X₇₈₁₄ | M8 | 69 | 2240834 | C | T | [symbol] | CED84277 |
| X₇₈₁₅ | M8 | 69 | 2245069 | G | A | [symbol] | CED84279 |
| X₇₈₁₆ | M8 | 69 | 2264484 | G | A | [symbol] | CED84284 |
| X₇₈₁₇ | M8 | 69 | 2283903 | G | A | [symbol] | CED84293 |
| X₇₈₁₈ | M8 | 69 | 2366082 | C | T | [symbol] | CED84318 |
| X₇₈₁₉ | M8 | 69 | 2438727 | C | T | [symbol] | CED84343 |
| X₇₈₂₀ | M8 | 69 | 2483433 | G | A | [symbol] | CED84352 |
| X₇₈₂₁ | M8 | 72 | 93 | T | C | [symbol] | |
| X₇₈₂₂ | M8 | 72 | 584 | G | A | [symbol] | |
| X₇₈₂₃ | M8 | 72 | 856 | C | T | [symbol] | |
| X₇₈₂₄ | M8 | 72 | 1163 | C | T | [symbol] | |
| X₇₈₂₅ | M8 | 73 | 730 | C | A | [symbol] | CDZ96628 |
| X₇₈₂₆ | M8 | 77 | 18912 | A | G | [symbol] | CDZ96635 |
| X₇₈₂₇ | M8 | 77 | 34978 | G | T | [symbol] | CDZ96642 |
| X₇₈₂₈ | M8 | 77 | 39842 | G | A | [symbol] | CDZ96646 |
| X₇₈₂₉ | M8 | 77 | 84022 | G | A | [symbol] | CDZ96659 |
| X₇₈₃₀ | M8 | 77 | 116691 | G | A | [symbol] | CDZ96671 |
| X₇₈₃₁ | M8 | 78 | 1865 | C | T | [symbol] | CED84359 |
| X₇₈₃₂ | M8 | 78 | 6333 | G | A | [symbol] | CED84360 |
| X₇₈₃₃ | M8 | 78 | 21716 | C | T | [symbol] | CED84364 |
| X₇₈₃₄ | M8 | 78 | 35200 | G | A | [symbol] | CED84371 |
| X₇₈₃₅ | M8 | 78 | 109325 | G | A | [symbol] | CED84390 |
| X₇₈₃₆ | M8 | 78 | 127873 | G | A | [symbol] | CED84395 |
| X₇₈₃₇ | M8 | 78 | 158434 | G | A | [symbol] | CED84406 |
| X₇₈₃₈ | M8 | 78 | 158534 | G | A | [symbol] | CED84406 |
| X₇₈₃₉ | M8 | 78 | 200234 | C | T | [symbol] | CED84420 |
| X₇₈₄₀ | M8 | 78 | 238854 | C | T | [symbol] | CED84430 |
| X₇₈₄₁ | M8 | 78 | 262614 | A | C | [symbol] | CED84438 |
| X₇₈₄₂ | M8 | 78 | 262683 | G | A | [symbol] | CED84438 |
| X₇₈₄₃ | M8 | 78 | 263826 | C | T | [symbol] | CED84438 |
| X₇₈₄₄ | M8 | 78 | 263828 | T | C | [symbol] | CED84438 |
| X₇₈₄₅ | M8 | 78 | 281454 | C | A | [symbol] | CED84441 |
| X₇₈₄₆ | M8 | 78 | 292068 | C | T | [symbol] | CED84442 |
| X₇₈₄₇ | M8 | 78 | 292139 | A | T | [symbol] | CED84442 |
| X₇₈₄₈ | M8 | 78 | 292243 | G | A | [symbol] | CED84442 |
| X₇₈₄₉ | M8 | 78 | 298141 | C | G | [symbol] | CED84443 |
| X₇₈₅₀ | M8 | 78 | 299644 | G | A | [symbol] | CED84443 |
| X₇₈₅₁ | M8 | 78 | 301815 | T | C | [symbol] | CED84443 |
| X₇₈₅₂ | M8 | 78 | 304799 | A | C | [symbol] | CED84444 |
| X₇₈₅₃ | M8 | 78 | 311586 | C | T | [symbol] | CED84447 |
| X₇₈₅₄ | M8 | 78 | 333312 | A | G | [symbol] | CED84452 |
| X₇₈₅₅ | M8 | 78 | 368785 | G | A | [symbol] | CED84464 |
| X₇₈₅₆ | M8 | 78 | 403167 | G | A | [symbol] | CED84476 |
| X₇₈₅₇ | M8 | 78 | 404944 | G | A | [symbol] | CED84475 |
| X₇₈₅₈ | M8 | 78 | 439640 | C | T | [symbol] | CED84487 |
| X₇₈₅₉ | M8 | 78 | 462728 | G | A | [symbol] | CED84497 |
| X₇₈₆₀ | M8 | 78 | 480848 | C | T | [symbol] | CED84500 |
| X₇₈₆₁ | M8 | 78 | 506703 | G | A | [symbol] | CED84508 |
| X₇₈₆₂ | M8 | 78 | 534530 | C | T | [symbol] | CED84520 |
| X₇₈₆₃ | M8 | 78 | 611362 | C | T | [symbol] | CED84546 |
| X₇₈₆₄ | M8 | 78 | 626958 | C | A | [symbol] | CED84549 |
| X₇₈₆₅ | M8 | 78 | 631891 | C | T | [symbol] | CED84551 |
| X₇₈₆₆ | M8 | 78 | 726983 | G | A | [symbol] | CED84587 |
| X₇₈₆₇ | M8 | 78 | 763771 | G | A | [symbol] | CED84600 |
| X₇₈₆₈ | M8 | 78 | 774252 | G | A | [symbol] | CED84602 |
| X₇₈₆₉ | M8 | 78 | 847242 | G | T | [symbol] | CED84624 |
| X₇₈₇₀ | M8 | 78 | 905457 | A | G | [symbol] | CED84645 |
| X₇₈₇₁ | M8 | 78 | 952815 | G | A | [symbol] | CED84662 |
| X₇₈₇₂ | M8 | 78 | 952860 | G | A | [symbol] | CED84662 |
| X₇₈₇₃ | M8 | 78 | 1023381 | G | A | [symbol] | CED84688 |
| X₇₈₇₄ | M8 | 78 | 1086537 | C | T | [symbol] | CED84713 |
| X₇₈₇₅ | M8 | 78 | 1095322 | C | T | [symbol] | CED84715 |
| X₇₈₇₆ | M8 | 78 | 1112587 | C | T | [symbol] | CED84722 |
| X₇₈₇₇ | M8 | 78 | 1115511 | G | A | [symbol] | CED84723 |
| X₇₈₇₈ | M8 | 78 | 1199323 | C | T | [symbol] | CED84746 |
| X₇₈₇₉ | M8 | 78 | 1216573 | C | T | [symbol] | CED84752 |
| X₇₈₈₀ | M8 | 78 | 1247678 | C | T | [symbol] | CED84761 |
| X₇₈₈₁ | M8 | 78 | 1278982 | C | T | [symbol] | CED84773 |
| X₇₈₈₂ | M8 | 78 | 1302286 | C | T | [symbol] | CED84782 |
| X₇₈₈₃ | M8 | 78 | 1317124 | G | A | [symbol] | CED84787 |
| X₇₈₈₄ | M8 | 78 | 1344640 | C | T | [symbol] | CED84795 |
| X₇₈₈₅ | M8 | 78 | 1356873 | C | T | [symbol] | CED84800 |
| X₇₈₈₆ | M8 | 78 | 1376517 | G | A | [symbol] | CED84810 |
| X₇₈₈₇ | M8 | 78 | 1406981 | C | T | [symbol] | CED84821 |
| X₇₈₈₈ | M8 | 78 | 1426722 | C | T | [symbol] | CED84829 |
| X₇₈₈₉ | M8 | 78 | 1475803 | G | A | [symbol] | CED84845 |
| X₇₈₉₀ | M8 | 78 | 1495995 | C | T | [symbol] | CED84852 |
| X₇₈₉₁ | M8 | 78 | 1498179 | G | A | [symbol] | CED84852 |
| X₇₈₉₂ | M8 | 78 | 1520586 | C | T | [symbol] | CED84855 |
| X₇₈₉₃ | M8 | 78 | 1549659 | G | A | [symbol] | CED84862 |
| X₇₈₉₄ | M8 | 78 | 1568326 | C | T | [symbol] | CED84868 |
| X₇₈₉₅ | M8 | 78 | 1578844 | G | A | [symbol] | CED84871 |
| X₇₈₉₆ | M8 | 78 | 1642170 | G | A | [symbol] | CED84889 |
| X₇₈₉₇ | M8 | 78 | 1660408 | G | A | [symbol] | CED84897 |
| X₇₈₉₈ | M8 | 78 | 1694684 | C | T | [symbol] | CED84912 |
| X₇₈₉₉ | M8 | 78 | 1742796 | G | A | [symbol] | CED84926 |
| X₇₉₀₀ | M8 | 79 | 161 | T | C | [symbol] | CDZ96674 |
| X₇₉₀₁ | M8 | 79 | 6477 | T | C | [symbol] | CDZ96674- |
| | | | | | | | CDZ96675 |
| X₇₉₀₂ | M8 | 79 | 37870 | T | C | [symbol] | CDZ96683 |
| X₇₉₀₃ | M8 | 79 | 47085 | G | A | [symbol] | CDZ96684 |
| X₇₉₀₄ | M8 | 79 | 57804 | G | A | [symbol] | CDZ96687 |
| X₇₉₀₅ | M8 | 79 | 64658 | C | T | [symbol] | CDZ96688 |
| X₇₉₀₆ | M8 | 79 | 132683 | C | T | [symbol] | CDZ96713 |
| X₇₉₀₇ | M8 | 79 | 168570 | C | T | [symbol] | CDZ96727 |
| X₇₉₀₈ | M8 | 79 | 255486 | C | T | [symbol] | CDZ96758 |
| X₇₉₀₉ | M8 | 79 | 255780 | G | A | [symbol] | CDZ96757 |
| X₇₉₁₀ | M8 | 79 | 307021 | C | T | [symbol] | CDZ96777 |
| X₇₉₁₁ | M8 | 79 | 318883 | G | A | [symbol] | CDZ96779 |
| X₇₉₁₂ | M8 | 79 | 346823 | G | A | [symbol] | CDZ96789 |
| X₇₉₁₃ | M8 | 79 | 406184 | G | A | [symbol] | CDZ96808 |
| X₇₉₁₄ | M8 | 79 | 409085 | C | T | [symbol] | CDZ96810 |
| X₇₉₁₅ | M8 | 79 | 484404 | C | T | [symbol] | CDZ96840 |
| X₇₉₁₆ | M8 | 79 | 492523 | C | T | [symbol] | CDZ96842 |
| X₇₉₁₇ | M8 | 79 | 526910 | C | T | [symbol] | CDZ96854 |
| X₇₉₁₈ | M8 | 79 | 552798 | C | T | [symbol] | CDZ96861 |
| X₇₉₁₉ | M8 | 79 | 570874 | C | T | [symbol] | CDZ96867 |
| X₇₉₂₀ | M8 | 79 | 575411 | C | T | [symbol] | CDZ96868 |
| X₇₉₂₁ | M8 | 79 | 579674 | G | A | [symbol] | CDZ96871 |
| X₇₉₂₂ | M8 | 79 | 617057 | C | T | [symbol] | CDZ96882 |
| X₇₉₂₃ | M8 | 79 | 679140 | C | T | [symbol] | CDZ96904 |
| X₇₉₂₄ | M8 | 79 | 683546 | T | C | [symbol] | CDZ96904 |
| X₇₉₂₅ | M8 | 79 | 683550 | A | G | [symbol] | CDZ96904 |
| X₇₉₂₆ | M8 | 79 | 691309 | C | T | [symbol] | CDZ96911 |
| X₇₉₂₇ | M8 | 79 | 696174 | G | A | [symbol] | CDZ96911 |
| X₇₉₂₈ | M8 | 79 | 710422 | G | A | [symbol] | CDZ96916 |
| X₇₉₂₉ | M8 | 79 | 735053 | T | C | [symbol] | CDZ96928 |
| X₇₉₃₀ | M8 | 79 | 758025 | G | A | [symbol] | CDZ96931 |
| X₇₉₃₁ | M8 | 79 | 758078 | T | C | [symbol] | CDZ96931 |
| X₇₉₃₂ | M8 | 79 | 759403 | C | T | [symbol] | CDZ96931 |
| X₇₉₃₃ | M8 | 79 | 759589 | G | A | [symbol] | CDZ96932 |
| X₇₉₃₄ | M8 | 79 | 759659 | G | A | [symbol] | CDZ96931 |
| X₇₉₃₅ | M8 | 79 | 760430 | T | C | [symbol] | CDZ96932 |
| X₇₉₃₆ | M8 | 79 | 760541 | A | G | [symbol] | CDZ96932 |
| X₇₉₃₇ | M8 | 79 | 760891 | A | G | [symbol] | CDZ96932 |
| X₇₉₃₈ | M8 | 79 | 760904 | T | C | [symbol] | CDZ96932 |
| X₇₉₃₉ | M8 | 79 | 760988 | G | T | [symbol] | CDZ96932 |
| X₇₉₄₀ | M8 | 79 | 761056 | T | C | [symbol] | CDZ96932 |
| X₇₉₄₁ | M8 | 79 | 761210 | T | C | [symbol] | CDZ96932 |
| X₇₉₄₂ | M8 | 79 | 768537 | A | G | [symbol] | CDZ96933 |
| X₇₉₄₃ | M8 | 79 | 776112 | A | G | [symbol] | CDZ96934 |
| X₇₉₄₄ | M8 | 79 | 776301 | C | A | [symbol] | CDZ96934 |
| X₇₉₄₅ | M8 | 79 | 776304 | G | T | [symbol] | CDZ96934 |
| X₇₉₄₆ | M8 | 79 | 780012 | T | C | [symbol] | CDZ96934 |
| X₇₉₄₇ | M8 | 79 | 786064 | A | G | [symbol] | CDZ96934 |
| X₇₉₄₈ | M8 | 79 | 798799 | C | T | [symbol] | CDZ96939 |
| X₇₉₄₉ | M8 | 79 | 973493 | C | T | [symbol] | CDZ97000 |
| X₇₉₅₀ | M8 | 79 | 993074 | C | T | [symbol] | CDZ97006 |
| X₇₉₅₁ | M8 | 79 | 1073437 | C | A | [symbol] | CDZ97033 |
| X₇₉₅₂ | M8 | 79 | 1123288 | C | T | [symbol] | CDZ97049 |
| X₇₉₅₃ | M8 | 79 | 1140763 | G | A | [symbol] | CDZ97053 |
| X₇₉₅₄ | M8 | 79 | 1209208 | G | A | [symbol] | CDZ97077 |
| X₇₉₅₅ | M8 | 79 | 1211985 | G | A | [symbol] | CDZ97077 |
| X₇₉₅₆ | M8 | 79 | 1229129 | G | A | [symbol] | CDZ97086 |
| X₇₉₅₇ | M8 | 79 | 1231740 | C | T | [symbol] | CDZ97087 |
| X₇₉₅₈ | M8 | 79 | 1254757 | G | A | [symbol] | CDZ97095 |
| X₇₉₅₉ | M8 | 79 | 1318034 | C | T | [symbol] | CDZ97117 |
| X₇₉₆₀ | M8 | 79 | 1336766 | C | T | [symbol] | CDZ97121 |
| X₇₉₆₁ | M8 | 79 | 1344338 | G | A | [symbol] | CDZ97124 |
| X₇₉₆₂ | M8 | 79 | 1371491 | C | T | [symbol] | CDZ97129 |
| X₇₉₆₃ | M8 | 79 | 1430954 | G | A | [symbol] | CDZ97152 |
| X₇₉₆₄ | M8 | 79 | 1494219 | T | A | [symbol] | CDZ97172 |
| X₇₉₆₅ | M8 | 79 | 1540205 | C | T | [symbol] | CDZ97186 |
| X₇₉₆₆ | M8 | 79 | 1541574 | G | A | [symbol] | CDZ97186 |
| X₇₉₆₇ | M8 | 79 | 1624415 | G | A | [symbol] | CDZ97217 |
| X₇₉₆₈ | M8 | 79 | 1658469 | C | T | [symbol] | CDZ97227 |
| X₇₉₆₉ | M8 | 79 | 1674448 | G | A | [symbol] | CDZ97228 |
| X₇₉₇₀ | M8 | 79 | 1694516 | C | T | [symbol] | CDZ97233 |
| X₇₉₇₁ | M8 | 79 | 1696776 | G | A | [symbol] | CDZ97234 |
| X₇₉₇₂ | M8 | 79 | 1738540 | A | T | [symbol] | CDZ97247 |
| X₇₉₇₃ | M8 | 79 | 1772244 | G | A | [symbol] | CDZ97258 |
| X₇₉₇₄ | M8 | 79 | 1788096 | G | A | [symbol] | CDZ97262 |
| X₇₉₇₅ | M8 | 79 | 1791482 | G | A | [symbol] | CDZ97263 |
| X₇₉₇₆ | M8 | 79 | 1797660 | C | T | [symbol] | CDZ97265 |
| X₇₉₇₇ | M8 | 79 | 1797676 | G | A | [symbol] | CDZ97265 |
| X₇₉₇₈ | M8 | 79 | 1809211 | C | T | [symbol] | CDZ97268 |
| X₇₉₇₉ | M8 | 79 | 1842448 | G | A | [symbol] | CDZ97282 |
| X₇₉₈₀ | M8 | 79 | 1855037 | A | G | [symbol] | CDZ97285 |
| X₇₉₈₁ | M8 | 79 | 1859220 | A | G | [symbol] | CDZ97291 |
| X₇₉₈₂ | M8 | 79 | 1863178 | A | T | [symbol] | CDZ97292 |
| X₇₉₈₃ | M8 | 79 | 1930688 | G | A | [symbol] | CDZ97315 |
| X₇₉₈₄ | M8 | 79 | 2001789 | C | T | [symbol] | CDZ97339 |
| X₇₉₈₅ | M8 | 79 | 2002072 | C | T | [symbol] | CDZ97339 |
| X₇₉₈₆ | M8 | 79 | 2013361 | C | T | [symbol] | CDZ97344 |
| X₇₉₈₇ | M8 | 79 | 2113064 | G | A | [symbol] | CDZ97381 |
| X₇₉₈₈ | M8 | 79 | 2115870 | G | A | [symbol] | CDZ97380 |
| X₇₉₈₉ | M8 | 79 | 2121041 | G | A | [symbol] | CDZ97380 |
| X₇₉₉₀ | M8 | 79 | 2152573 | C | T | [symbol] | CDZ97392 |
| X₇₉₉₁ | M8 | 79 | 2172085 | C | T | [symbol] | CDZ97397 |
| X₇₉₉₂ | M8 | 79 | 2182096 | G | A | [symbol] | CDZ97403 |
| X₇₉₉₃ | M8 | 79 | 2188410 | G | A | [symbol] | CDZ97405 |
| X₇₉₉₄ | M8 | 79 | 2220254 | C | T | [symbol] | CDZ97414 |
| X₇₉₉₅ | M8 | 79 | 2268540 | C | T | [symbol] | CDZ97435 |
| X₇₉₉₆ | M8 | 79 | 2304361 | G | A | [symbol] | CDZ97450 |
| X₇₉₉₇ | M8 | 79 | 2317043 | G | A | [symbol] | CDZ97452 |
| X₇₉₉₈ | M8 | 79 | 2339881 | C | G | [symbol] | CDZ97460 |
| X₇₉₉₉ | M8 | 79 | 2340326 | G | A | [symbol] | CDZ97462 |
| X₈₀₀₀ | M8 | 85 | 462 | G | A | [symbol] | |
| X₈₀₀₁ | M8 | 85 | 463 | A | G | [symbol] | |
| X₈₀₀₂ | M8 | 85 | 467 | A | T | [symbol] | |
| X₈₀₀₃ | M8 | 85 | 512 | A | G | [symbol] | |
| X₈₀₀₄ | M8 | 85 | 516 | G | A | [symbol] | |
| X₈₀₀₅ | M8 | 85 | 521 | C | T | [symbol] | |
| X₈₀₀₆ | M8 | 85 | 522 | A | G | [symbol] | |
| X₈₀₀₇ | M8 | 88 | 119 | C | T | [symbol] | CDZ97464 |
| X₈₀₀₈ | M8 | 88 | 196 | G | A | [symbol] | CDZ97464 |
| X₈₀₀₉ | M8 | 88 | 898 | C | T | [symbol] | CDZ97464 |
| X₈₀₁₀ | M8 | 99 | 245 | G | A | [symbol] | |
| X₈₀₁₁ | M8 | 105 | 429 | C | T | [symbol] | CDZ97474 |
| X₈₀₁₂ | M8 | 109 | 396 | T | C | [symbol] | |
| X₈₀₁₃ | M8 | 113 | 411 | G | A | [symbol] | |
| X₈₀₁₄ | M8 | 118 | 396 | C | T | [symbol] | CDZ97478 |
| X₈₀₁₅ | M8 | 118 | 870 | G | A | [symbol] | CDZ97478 |
| X₈₀₁₆ | M8 | 118 | 1491 | G | A | [symbol] | CDZ97478 |
| X₈₀₁₇ | M8 | 121 | 318 | A | T | [symbol] | |
| X₈₀₁₈ | M8 | 124 | 72117 | G | A | [symbol] | CDZ97501 |
| X₈₀₁₉ | M8 | 124 | 72760 | G | A | [symbol] | CDZ97501 |
| X₈₀₂₀ | M8 | 124 | 94611 | G | A | [symbol] | CDZ97507 |
| X₈₀₂₁ | M8 | 124 | 156052 | G | A | [symbol] | CDZ97524 |
| X₈₀₂₂ | M8 | 124 | 166988 | C | T | [symbol] | CDZ97527 |
| X₈₀₂₃ | M8 | 124 | 207992 | C | T | [symbol] | CDZ97541 |
| X₈₀₂₄ | M8 | 124 | 222249 | C | T | [symbol] | CDZ97547 |
| X₈₀₂₅ | M8 | 125 | 35864 | T | C | [symbol] | CDZ97573 |
| X₈₀₂₆ | M8 | 126 | 104 | C | T | [symbol] | |
| X₈₀₂₇ | M8 | 129 | 299 | G | A | [symbol] | CDZ97577 |
| X₈₀₂₈ | M8 | 129 | 433 | G | A | [symbol] | CDZ97577 |
| X₈₀₂₉ | M8 | 129 | 856 | G | A | [symbol] | CDZ97577 |
| X₈₀₃₀ | M8 | 129 | 1013 | C | T | [symbol] | CDZ97577 |
| X₈₀₃₁ | M8 | 130 | 674 | C | T | [symbol] | CDZ97578 |
| X₈₀₃₂ | M8 | 141 | 5931 | G | A | [symbol] | CDZ97582 |
| X₈₀₃₃ | M8 | 141 | 15764 | C | T | [symbol] | CDZ97587 |
| X₈₀₃₄ | M8 | 141 | 17894 | G | A | [symbol] | CDZ97587 |
| X₈₀₃₅ | M8 | 141 | 25126 | C | T | [symbol] | CDZ97591 |
| X₈₀₃₆ | M8 | 149 | 1663 | A | G | [symbol] | CDZ97598 |
| X₈₀₃₇ | M8 | 149 | 2074 | T | G | [symbol] | CDZ97598 |
| X₈₀₃₈ | M8 | 152 | 141 | G | A | [symbol] | |
| X₈₀₃₉ | M8 | 159 | 106 | A | C | [symbol] | |
| X₈₀₄₀ | M8 | 162 | 70025 | C | T | [symbol] | CDZ97625 |
| X₈₀₄₁ | M8 | 162 | 70405 | A | G | [symbol] | CDZ97625 |
| X₈₀₄₂ | M8 | 162 | 93443 | G | A | [symbol] | CDZ97631 |
| X₈₀₄₃ | M8 | 162 | 199495 | G | A | [symbol] | CDZ97664 |
| X₈₀₄₄ | M8 | 162 | 207179 | C | T | [symbol] | CDZ97668 |
| X₈₀₄₅ | M8 | 162 | 207236 | G | A | [symbol] | CDZ97666 |
| X₈₀₄₆ | M8 | 162 | 223407 | C | T | [symbol] | CDZ97674 |
| X₈₀₄₇ | M8 | 162 | 233092 | C | T | [symbol] | CDZ97676 |
| X₈₀₄₈ | M8 | 162 | 268123 | G | A | [symbol] | CDZ97688 |
| X₈₀₄₉ | M8 | 162 | 291566 | G | A | [symbol] | CDZ97695 |
| X₈₀₅₀ | M8 | 162 | 304976 | A | G | [symbol] | CDZ97701 |
| X₈₀₅₁ | M8 | 162 | 308369 | T | C | [symbol] | CDZ97702 |
| X₈₀₅₂ | M8 | 162 | 321460 | C | G | [symbol] | CDZ97703 |
| X₈₀₅₃ | M8 | 162 | 321463 | A | C | [symbol] | CDZ97703 |
| X₈₀₅₄ | M8 | 162 | 322591 | G | A | [symbol] | CDZ97703 |
| X₈₀₅₅ | M8 | 162 | 324114 | T | C | [symbol] | CDZ97703 |
| X₈₀₅₆ | M8 | 162 | 324139 | C | T | [symbol] | CDZ97703 |
| X₈₀₅₇ | M8 | 16 | 324568 | A | G | [symbol] | CDZ97703 |
| X₈₀₅₈ | M8 | 162 | 324604 | G | A | [symbol] | CDZ97703 |
| X₈₀₅₉ | M8 | 162 | 351016 | C | T | [symbol] | CDZ97711 |
| X₈₀₆₀ | M8 | 162 | 360320 | G | A | [symbol] | CDZ97714 |
| X₈₀₆₁ | M8 | 162 | 424391 | C | T | [symbol] | CDZ97740 |
| X₈₀₆₂ | M8 | 162 | 444123 | T | C | [symbol] | CDZ97748 |
| X₈₀₆₃ | M8 | 162 | 457645 | G | A | [symbol] | CDZ97752 |
| X₈₀₆₄ | M8 | 162 | 467207 | C | T | [symbol] | CDZ97755 |
| X₈₀₆₅ | M8 | 162 | 476267 | C | T | [symbol] | CDZ97760 |
| X₈₀₆₆ | M8 | 162 | 491906 | G | A | [symbol] | CDZ97764 |
| X₈₀₆₇ | M8 | 162 | 492101 | G | A | [symbol] | CDZ97764 |
| X₈₀₆₈ | M8 | 162 | 501886 | C | T | [symbol] | CDZ97769 |
| X₈₀₆₉ | M8 | 162 | 541612 | G | A | [symbol] | CDZ97784 |
| X₈₀₇₀ | M8 | 162 | 632311 | G | A | [symbol] | CDZ97814 |
| X₈₀₇₁ | M8 | 162 | 633321 | G | A | [symbol] | CDZ97814 |
| X₈₀₇₂ | M8 | 162 | 633633 | G | A | [symbol] | CDZ97814 |
| X₈₀₇₃ | M8 | 162 | 638083 | C | T | [symbol] | CDZ97814 |
| X₈₀₇₄ | M8 | 162 | 640005 | C | T | [symbol] | CDZ97815 |
| X₈₀₇₅ | M8 | 162 | 674192 | C | T | [symbol] | CDZ97824 |
| X₈₀₇₆ | M8 | 162 | 680645 | G | A | [symbol] | CDZ97826 |
| X₈₀₇₇ | M8 | 162 | 738675 | G | A | [symbol] | CDZ97842 |
| X₈₀₇₈ | M8 | 162 | 738686 | C | T | [symbol] | CDZ97842 |
| X₈₀₇₉ | M8 | 162 | 808830 | G | A | [symbol] | CDZ97869 |
| X₈₀₈₀ | M8 | 162 | 861370 | G | T | [symbol] | CDZ97889 |
| X₈₀₈₁ | M8 | 162 | 871911 | C | T | [symbol] | CDZ97890 |
| X₈₀₈₂ | M8 | 162 | 914850 | C | T | [symbol] | CDZ97904 |
| X₈₀₈₃ | M8 | 162 | 966019 | C | T | [symbol] | CDZ97923 |
| X₈₀₈₄ | M8 | 162 | 972973 | G | A | [symbol] | CDZ97925 |
| X₈₀₈₅ | M8 | 162 | 997234 | C | T | [symbol] | CDZ97933 |
| X₈₀₈₆ | M8 | 175 | 385 | A | C | [symbol] | |
| X₈₀₈₇ | M8 | 185 | 758 | C | T | [symbol] | CDZ97958 |
| X₈₀₈₈ | M8 | 189 | 1306 | C | T | [symbol] | CDZ97961 |
| X₈₀₈₉ | M8 | 189 | 126573 | G | A | [symbol] | CDZ97996 |
| X₈₀₉₀ | M8 | 189 | 127356 | G | A | [symbol] | CDZ97996 |
| X₈₀₉₁ | M8 | 189 | 149091 | G | A | [symbol] | CDZ98004 |
| X₈₀₉₂ | M8 | 189 | 174918 | G | A | [symbol] | CDZ98015 |
| X₈₀₉₃ | M8 | 189 | 194647 | A | G | [symbol] | CDZ98020 |
| X₈₀₉₄ | M8 | 189 | 194665 | A | G | [symbol] | CDZ98020 |
| X₈₀₉₅ | M8 | 189 | 212649 | C | T | [symbol] | CDZ98028 |
| X₈₀₉₆ | M8 | 189 | 257670 | C | T | [symbol] | CDZ98041 |
| X₈₀₉₇ | M8 | 189 | 300343 | C | T | [symbol] | CDZ98056 |
| X₈₀₉₈ | M8 | 189 | 323680 | C | T | [symbol] | CDZ98063 |
| X₈₀₉₉ | M8 | 189 | 332957 | G | A | [symbol] | CDZ98065 |
| X₈₁₀₀ | M8 | 189 | 371247 | G | A | [symbol] | CDZ98078 |
| X₈₁₀₁ | M8 | 191 | 342 | A | G | [symbol] | CDZ98107 |
| X₈₁₀₂ | M8 | 191 | 347 | C | T | [symbol] | CDZ98107 |
| X₈₁₀₃ | M8 | 191 | 427 | C | T | [symbol] | CDZ98107 |
| X₈₁₀₄ | M8 | 191 | 451 | C | T | [symbol] | CDZ98107 |
| X₈₁₀₅ | M8 | 191 | 487 | A | G | [symbol] | CDZ98107 |
| X₈₁₀₆ | M8 | 197 | 624 | C | T | [symbol] | CDZ98111 |
| X₈₁₀₇ | M8 | 198 | 202 | C | A | [symbol] | |
| X₈₁₀₈ | M8 | 198 | 203 | G | A | [symbol] | |
| X₈₁₀₉ | M8 | 198 | 238 | C | T | [symbol] | |
| X₈₁₁₀ | M8 | 198 | 265 | C | T | [symbol] | |
| X₈₁₁₁ | M8 | 199 | 688 | C | T | [symbol] | |
| X₈₁₁₂ | M8 | 199 | 911 | C | T | [symbol] | |
| X₈₁₁₃ | M8 | 199 | 971 | C | T | [symbol] | |
| X₈₁₁₄ | M8 | 199 | 1218 | C | T | [symbol] | |
| X₈₁₁₅ | M8 | 202 | 171 | C | T | [symbol] | |
| X₈₁₁₆ | M8 | 205 | 360 | C | T | [symbol] | CDZ98112 |
| X₈₁₁₇ | M8 | 205 | 981 | C | T | [symbol] | CDZ98112 |
| X₈₁₁₈ | M8 | 208 | 117 | G | A | [symbol] | |
| X₈₁₁₉ | M8 | 208 | 476 | G | A | [symbol] | |
| X₈₁₂₀ | M8 | 208 | 616 | G | A | [symbol] | |
| X₈₁₂₁ | M8 | 208 | 935 | G | A | [symbol] | |
| X₈₁₂₂ | M8 | 209 | 312 | C | T | [symbol] | CDZ98113 |
| X₈₁₂₃ | M8 | 21 | 197 | C | G | [symbol] | CDZ98116 |
| X₈₁₂₄ | M8 | 223 | 93 | G | A | [symbol] | CDZ98118 |
| X₈₁₂₅ | M8 | 223 | 102 | A | G | [symbol] | CDZ98118 |
| X₈₁₂₆ | M8 | 223 | 133 | A | G | [symbol] | CDZ98118 |
| X₈₁₂₇ | M8 | 223 | 150 | T | C | [symbol] | CDZ98118 |
| X₈₁₂₈ | M8 | 223 | 155 | A | C | [symbol] | CDZ98118 |
| X₈₁₂₉ | M8 | 223 | 158 | T | C | [symbol] | CDZ98118 |
| X₈₁₃₀ | M8 | 223 | 160 | C | T | [symbol] | CDZ98118 |
| X₈₁₃₁ | M8 | 223 | 175 | G | A | [symbol] | CDZ98118 |
| X₈₁₃₂ | M8 | 223 | 177 | A | G | [symbol] | CDZ98118 |
| X₈₁₃₃ | M8 | 22 | 291 | C | A | [symbol] | CDZ98118 |
| X₈₁₃₄ | M8 | 225 | 317 | C | T | [symbol] | |
| X₈₁₃₅ | M8 | 227 | 202 | G | A | [symbol] | |
| X₈₁₃₆ | M8 | 232 | 340 | G | A | [symbol] | CDZ98119 |
| X₈₁₃₇ | M8 | 232 | 1379 | C | T | [symbol] | CDZ98119 |
| X₈₁₃₈ | M8 | 232 | 1521 | C | T | [symbol] | CDZ98119 |
| X₈₁₃₉ | M8 | 232 | 1662 | G | A | [symbol] | CDZ98119 |
| X₈₁₄₀ | M8 | 232 | 1761 | C | T | [symbol] | CDZ98119 |
| X₈₁₄₁ | M8 | 232 | 2027 | C | T | [symbol] | CDZ98119 |
| X₈₁₄₂ | M8 | 232 | 3060 | C | T | [symbol] | CDZ98120 |
| X₈₁₄₃ | M8 | 232 | 3284 | C | T | [symbol] | CDZ98119 |
| X₈₁₄₄ | M8 | 232 | 4437 | G | A | [symbol] | CDZ98121 |
| X₈₁₄₅ | M8 | 232 | 4561 | C | T | [symbol] | CDZ98120 |
| X₈₁₄₆ | M8 | 242 | 7334 | C | T | [symbol] | CDZ98125 |
| X₈₁₄₇ | M8 | 242 | 41710 | G | A | [symbol] | CDZ98131 |
| X₈₁₄₈ | M8 | 242 | 50262 | G | A | [symbol] | CDZ98135 |
| X₈₁₄₉ | M8 | 242 | 57480 | C | T | [symbol] | CDZ98137 |
| X₈₁₅₀ | M8 | 242 | 88037 | G | A | [symbol] | CDZ98147 |
| X₈₁₅₁ | M8 | 242 | 107587 | G | A | [symbol] | CDZ98153 |
| X₈₁₅₂ | M8 | 242 | 112244 | G | A | [symbol] | CDZ98154 |
| X₈₁₅₃ | M8 | 242 | 147104 | G | A | [symbol] | CDZ98164 |
| X₈₁₅₄ | M8 | 242 | 155049 | C | T | [symbol] | CDZ98168 |
| X₈₁₅₅ | M8 | 242 | 168689 | G | A | [symbol] | CDZ98172 |
| X₈₁₅₆ | M8 | 242 | 194983 | C | T | [symbol] | CDZ98179 |
| X₈₁₅₇ | M8 | 242 | 213986 | G | A | [symbol] | CDZ98185 |
| X₈₁₅₈ | M8 | 242 | 219778 | C | T | [symbol] | CDZ98187 |
| X₈₁₅₉ | M8 | 242 | 227271 | C | T | [symbol] | CDZ98191 |
| X₈₁₆₀ | M8 | 242 | 233088 | G | A | [symbol] | CDZ98193 |
| X₈₁₆₁ | M8 | 242 | 263218 | G | T | [symbol] | CDZ98201 |
| X₈₁₆₂ | M8 | 242 | 263678 | G | T | [symbol] | CDZ98202 |
| X₈₁₆₃ | M8 | 242 | 310353 | G | A | [symbol] | CDZ98216 |
| X₈₁₆₄ | M8 | 242 | 376713 | G | A | [symbol] | CDZ98238 |
| X₈₁₆₅ | M8 | 242 | 404132 | G | A | [symbol] | CDZ98248 |
| X₈₁₆₆ | M8 | 242 | 405825 | G | A | [symbol] | CDZ98250 |
| X₈₁₆₇ | M8 | 242 | 412676 | G | A | [symbol] | CDZ98249 |
| X₈₁₆₈ | M8 | 242 | 437941 | G | A | [symbol] | CDZ98258 |
| X₈₁₆₉ | M8 | 242 | 485936 | G | A | [symbol] | CDZ98270 |
| X₈₁₇₀ | M8 | 242 | 492389 | G | A | [symbol] | CDZ98274 |
| X₈₁₇₁ | M8 | 242 | 499956 | A | G | [symbol] | CDZ98276 |
| X₈₁₇₂ | M8 | 242 | 514780 | C | T | [symbol] | CDZ98282 |
| X₈₁₇₃ | M8 | 242 | 542663 | G | A | [symbol] | CDZ98290 |
| X₈₁₇₄ | M8 | 242 | 559999 | G | A | [symbol] | CDZ98297 |
| X₈₁₇₅ | M8 | 242 | 602456 | G | A | [symbol] | CDZ98308 |
| X₈₁₇₆ | M8 | 242 | 602531 | G | A | [symbol] | CDZ98308 |
| X₈₁₇₇ | M8 | 242 | 604704 | C | T | [symbol] | CDZ98309 |
| X₈₁₇₈ | M8 | 242 | 630157 | C | T | [symbol] | CDZ98319 |
| X₈₁₇₉ | M8 | 249 | 5307 | C | T | [symbol] | CED84929 |
| X₈₁₈₀ | M8 | 249 | 17416 | A | G | [symbol] | CED84933 |
| X₈₁₈₁ | M8 | 249 | 72016 | C | T | [symbol] | CED84950 |
| X₈₁₈₂ | M8 | 249 | 137338 | G | A | [symbol] | CED84976 |
| X₈₁₈₃ | M8 | 249 | 157550 | C | T | [symbol] | CED84983 |
| X₈₁₈₄ | M8 | 249 | 171600 | G | A | [symbol] | CED84986 |
| X₈₁₈₅ | M8 | 249 | 191304 | C | T | [symbol] | CED84988 |
| X₈₁₈₆ | M8 | 249 | 212696 | G | A | [symbol] | CED84994 |
| X₈₁₈₇ | M8 | 249 | 253494 | C | T | [symbol] | CED85008 |
| X₈₁₈₈ | M8 | 249 | 272075 | C | T | [symbol] | CED85013 |
| X₈₁₈₉ | M8 | 249 | 285231 | C | T | [symbol] | CED85015 |
| X₈₁₉₀ | M8 | 249 | 294161 | G | A | [symbol] | CED85018 |
| X₈₁₉₁ | M8 | 249 | 305975 | G | A | [symbol] | CED85023 |
| X₈₁₉₂ | M8 | 249 | 311154 | G | A | [symbol] | CED85025 |
| X₈₁₉₃ | M8 | 249 | 359266 | C | T | [symbol] | CED85041 |
| X₈₁₉₄ | M8 | 249 | 371535 | G | A | [symbol] | CED85048 |
| X₈₁₉₅ | M8 | 249 | 377814 | G | A | [symbol] | CED85051 |
| X₈₁₉₆ | M8 | 249 | 420458 | C | T | [symbol] | CED85069 |
| X₈₁₉₇ | M8 | 249 | 485782 | C | T | [symbol] | CED85089 |
| X₈₁₉₈ | M8 | 249 | 500195 | C | T | [symbol] | CED85093 |
| X₈₁₉₉ | M8 | 249 | 501310 | C | T | [symbol] | CED85093 |
| X₈₂₀₀ | M8 | 249 | 501510 | T | C | [symbol] | CED85093 |
| X₈₂₀₁ | M8 | 249 | 509901 | C | T | [symbol] | CED85096 |
| X₈₂₀₂ | M8 | 249 | 532943 | A | G | [symbol] | CED85099 |
| X₈₂₀₃ | M8 | 249 | 576070 | G | A | [symbol] | CED85118 |
| X₈₂₀₄ | M8 | 249 | 598579 | C | T | [symbol] | CED85125 |
| X₈₂₀₅ | M8 | 249 | 626627 | G | A | [symbol] | CED85132 |
| X₈₂₀₆ | M8 | 249 | 637944 | G | A | [symbol] | CED85135 |
| X₈₂₀₇ | M8 | 249 | 660408 | G | A | [symbol] | CED85144 |
| X₈₂₀₈ | M8 | 249 | 715551 | G | A | [symbol] | CED85163 |
| X₈₂₀₉ | M8 | 249 | 751427 | G | A | [symbol] | CED85174 |
| X₈₂₁₀ | M8 | 249 | 803215 | C | T | [symbol] | CED85186 |
| X₈₂₁₁ | M8 | 249 | 805141 | T | A | [symbol] | CED85187 |
| X₈₂₁₂ | M8 | 249 | 854220 | G | A | [symbol] | CED85201 |
| X₈₂₁₃ | M8 | 249 | 871689 | G | A | [symbol] | CED85204 |
| X₈₂₁₄ | M8 | 249 | 889853 | G | A | [symbol] | CED85211 |
| X₈₂₁₅ | M8 | 249 | 911274 | C | T | [symbol] | CED85218 |
| X₈₂₁₆ | M8 | 249 | 927876 | C | T | [symbol] | CED85222 |
| X₈₂₁₇ | M8 | 249 | 932606 | C | T | [symbol] | CED85223 |
| X₈₂₁₈ | M8 | 249 | 934305 | G | A | [symbol] | CED85225 |
| X₈₂₁₉ | M8 | 249 | 935435 | C | T | [symbol] | CED85225 |
| X₈₂₂₀ | M8 | 249 | 945434 | C | T | [symbol] | CED85227 |
| X₈₂₂₁ | M8 | 249 | 945727 | C | T | [symbol] | CED85227 |
| X₈₂₂₂ | M8 | 249 | 969333 | G | T | [symbol] | CED85238 |
| X₈₂₂₃ | M8 | 249 | 977170 | C | T | [symbol] | CED85242 |
| X₈₂₂₄ | M8 | 249 | 983120 | T | A | [symbol] | CED85242 |
| X₈₂₂₅ | M8 | 249 | 989520 | C | T | [symbol] | CED85243 |
| X₈₂₂₆ | M8 | 249 | 1014098 | C | T | [symbol] | CED85252 |
| X₈₂₂₇ | M8 | 249 | 1021827 | C | T | [symbol] | CED85254 |
| X₈₂₂₈ | M8 | 249 | 1050123 | C | T | [symbol] | CED85262 |
| X₈₂₂₉ | M8 | 249 | 1062282 | G | A | [symbol] | CED85266 |
| X₈₂₃₀ | M8 | 249 | 1067266 | G | A | [symbol] | CED85267 |
| X₈₂₃₁ | M8 | 249 | 1075522 | G | A | [symbol] | CED85270 |
| X₈₂₃₂ | M8 | 249 | 1076333 | G | A | [symbol] | CED85270 |
| X₈₂₃₃ | M8 | 249 | 1089152 | G | A | [symbol] | CED85274 |
| X₈₂₃₄ | M8 | 249 | 1108583 | A | G | [symbol] | CED85281 |
| X₈₂₃₅ | M8 | 249 | 1114790 | G | A | [symbol] | CED85283 |
| X₈₂₃₆ | M8 | 249 | 1143228 | C | T | [symbol] | CED85292 |
| X₈₂₃₇ | M8 | 249 | 1144309 | G | A | [symbol] | CED85291 |
| X₈₂₃₈ | M8 | 249 | 1176977 | G | A | [symbol] | CED85303 |
| X₈₂₃₉ | M8 | 249 | 1195367 | G | A | [symbol] | CED85307 |
| X₈₂₄₀ | M8 | 249 | 1230068 | C | T | [symbol] | CED85319 |
| X₈₂₄₁ | M8 | 249 | 1299472 | C | T | [symbol] | CED85343 |
| X₈₂₄₂ | M8 | 249 | 1359399 | G | A | [symbol] | CED85360 |
| X₈₂₄₃ | M8 | 249 | 1391343 | G | A | [symbol] | CED85372 |
| X₈₂₄₄ | M8 | 249 | 1393971 | G | A | [symbol] | CED85373 |
| X₈₂₄₅ | M8 | 249 | 1457529 | G | A | [symbol] | CED85398 |
| X₈₂₄₆ | M8 | 249 | 1473400 | C | T | [symbol] | CED85404 |
| X₈₂₄₇ | M8 | 249 | 1475200 | G | A | [symbol] | CED85404 |
| X₈₂₄₈ | M8 | 249 | 1486325 | G | A | [symbol] | CED85409 |
| X₈₂₄₉ | M8 | 249 | 1498029 | T | C | [symbol] | CED85415 |
| X₈₂₅₀ | M8 | 249 | 1502631 | G | T | [symbol] | CED85416 |
| X₈₂₅₁ | M8 | 249 | 1502641 | T | A | [symbol] | CED85416 |
| X₈₂₅₂ | M8 | 249 | 1502819 | T | G | [symbol] | CED85416 |
| X₈₂₅₃ | M8 | 249 | 1505138 | G | A | [symbol] | CED85416 |
| X₈₂₅₄ | M8 | 249 | 1505634 | T | G | [symbol] | CED85416 |
| X₈₂₅₅ | M8 | 249 | 1519431 | A | G | [symbol] | CED85419 |
| X₈₂₅₆ | M8 | 249 | 1555472 | C | T | [symbol] | CED85428 |
| X₈₂₅₇ | M8 | 249 | 1619036 | G | A | [symbol] | CED85450 |
| X₈₂₅₈ | M8 | 249 | 1621516 | G | A | [symbol] | CED85451 |
| X₈₂₅₉ | M8 | 249 | 1623151 | G | A | [symbol] | CED85451 |
| X₈₂₆₀ | M8 | 249 | 1653615 | T | G | [symbol] | CED85457 |
| X₈₂₆₁ | M8 | 249 | 1653962 | C | T | [symbol] | CED85458 |
| X₈₂₆₂ | M8 | 249 | 1655479 | C | T | [symbol] | CED85458 |
| X₈₂₆₃ | M8 | 249 | 1666185 | C | T | [symbol] | CED85460 |
| X₈₂₆₄ | M8 | 249 | 1679615 | G | A | [symbol] | CED85461 |
| X₈₂₆₅ | M8 | 249 | 1726777 | C | T | [symbol] | CED85474 |
| X₈₂₆₆ | M8 | 249 | 1737431 | C | T | [symbol] | CED85478 |
| X₈₂₆₇ | M8 | 249 | 1780551 | A | G | [symbol] | CED85493 |
| X₈₂₆₈ | M8 | 249 | 1780569 | A | G | [symbol] | CED85493 |
| X₈₂₆₉ | M8 | 249 | 1782582 | G | T | [symbol] | CED85493 |
| X₈₂₇₀ | M8 | 249 | 1786050 | C | T | [symbol] | CED85496 |
| X₈₂₇₁ | M8 | 249 | 1801965 | G | A | [symbol] | CED85501 |
| X₈₂₇₂ | M8 | 249 | 1803575 | C | T | [symbol] | CED85502 |
| X₈₂₇₃ | M8 | 249 | 1803790 | G | A | [symbol] | CED85502 |
| X₈₂₇₄ | M8 | 249 | 1804835 | G | A | [symbol] | CED85502 |
| X₈₂₇₅ | M8 | 249 | 1834426 | G | A | [symbol] | CED85511 |
| X₈₂₇₆ | M8 | 249 | 1880762 | G | A | [symbol] | CED85525 |
| X₈₂₇₇ | M8 | 249 | 1944026 | G | A | [symbol] | CED85545 |
| X₈₂₇₈ | M8 | 249 | 1988505 | C | T | [symbol] | CED85560 |
| X₈₂₇₉ | M8 | 249 | 2010712 | G | A | [symbol] | CED85564 |
| X₈₂₈₀ | M8 | 249 | 2015272 | G | A | [symbol] | CED85567 |
| X₈₂₈₁ | M8 | 249 | 2027477 | G | A | [symbol] | CED85573 |
| X₈₂₈₂ | M8 | 249 | 2063467 | G | A | [symbol] | CED85586 |
| X₈₂₈₃ | M8 | 249 | 2110956 | C | T | [symbol] | CED85603 |
| X₈₂₈₄ | M8 | 249 | 2163304 | G | A | [symbol] | CED85618 |
| X₈₂₈₅ | M8 | 249 | 2170103 | C | T | [symbol] | CED85620 |
| X₈₂₈₆ | M8 | 249 | 2198205 | G | A | [symbol] | CED85628 |
| X₈₂₈₇ | M8 | 249 | 2223626 | G | A | [symbol] | CED85633 |
| X₈₂₈₈ | M8 | 249 | 2255547 | G | C | [symbol] | CED85640 |
| X₈₂₈₉ | M8 | 252 | 512 | C | T | [symbol] | |
| X₈₂₉₀ | M8 | 252 | 524 | C | T | [symbol] | |
| X₈₂₉₁ | M8 | 258 | 576 | A | G | [symbol] | |
| X₈₂₉₂ | M8 | 258 | 1139 | A | T | [symbol] | |
| X₈₂₉₃ | M8 | 260 | 156 | G | A | [symbol] | |
| X₈₂₉₄ | M8 | 262 | 4049 | G | A | [symbol] | CDZ98330 |
| X₈₂₉₅ | M8 | 262 | 30347 | G | A | [symbol] | CDZ98338 |
| X₈₂₉₆ | M8 | 262 | 30359 | G | A | [symbol] | CDZ98338 |
| X₈₂₉₇ | M8 | 262 | 39621 | C | T | [symbol] | CDZ98342 |
| X₈₂₉₈ | M8 | 262 | 109833 | C | T | [symbol] | CDZ98369 |
| X₈₂₉₉ | M8 | 262 | 130174 | C | T | [symbol] | CDZ98372 |
| X₈₃₀₀ | M8 | 262 | 138889 | C | T | [symbol] | CDZ98375 |
| X₈₃₀₁ | M8 | 262 | 144566 | G | A | [symbol] | CDZ98375 |
| X₈₃₀₂ | M8 | 262 | 148151 | C | T | [symbol] | CDZ98375 |
| X₈₃₀₃ | M8 | 262 | 164438 | G | A | [symbol] | CDZ98383 |
| X₈₃₀₄ | M8 | 262 | 191816 | C | T | [symbol] | CDZ98393 |
| X₈₃₀₅ | M8 | 262 | 201531 | G | A | [symbol] | CDZ98396 |
| X₈₃₀₆ | M8 | 262 | 226006 | C | A | [symbol] | CDZ98404 |
| X₈₃₀₇ | M8 | 262 | 233570 | G | A | [symbol] | CDZ98407 |
| X₈₃₀₈ | M8 | 262 | 252305 | G | A | [symbol] | CDZ98412 |
| X₈₃₀₉ | M8 | 262 | 319302 | G | A | [symbol] | CDZ98436 |
| X₈₃₁₀ | M8 | 262 | 327677 | C | T | [symbol] | CDZ98440 |
| X₈₃₁₁ | M8 | 262 | 366171 | G | A | [symbol] | CDZ98452 |
| X₈₃₁₂ | M8 | 262 | 373766 | C | T | [symbol] | CDZ98456 |
| X₈₃₁₃ | M8 | 262 | 380519 | C | T | [symbol] | CDZ98457 |
| X₈₃₁₄ | M8 | 262 | 390030 | C | T | [symbol] | CDZ98460 |
| X₈₃₁₅ | M8 | 262 | 397539 | G | A | [symbol] | CDZ98463 |
| X₈₃₁₆ | M8 | 262 | 410688 | C | T | [symbol] | CDZ98466 |
| X₈₃₁₇ | M8 | 262 | 418834 | C | T | [symbol] | CDZ98468 |
| X₈₃₁₈ | M8 | 262 | 420102 | G | A | [symbol] | CDZ98468 |
| X₈₃₁₉ | M8 | 262 | 446956 | C | T | [symbol] | CDZ98477 |
| X₈₃₂₀ | M8 | 262 | 471054 | C | T | [symbol] | CDZ98487 |
| X₈₃₂₁ | M8 | 262 | 498239 | C | T | [symbol] | CDZ98496 |
| X₈₃₂₂ | M8 | 262 | 539428 | G | A | [symbol] | CDZ98514 |
| X₈₃₂₃ | M8 | 262 | 539452 | A | G | [symbol] | CDZ98514 |
| X₈₃₂₄ | M8 | 262 | 539470 | A | G | [symbol] | CDZ98514 |
| X₈₃₂₅ | M8 | 262 | 539588 | A | G | [symbol] | CDZ98514 |
| X₈₃₂₆ | M8 | 262 | 552414 | C | T | [symbol] | CDZ98518 |
| X₈₃₂₇ | M8 | 262 | 557280 | G | A | [symbol] | CDZ98518 |
| X₈₃₂₈ | M8 | 262 | 560713 | G | A | [symbol] | CDZ98519 |
| X₈₃₂₉ | M8 | 262 | 562795 | C | T | [symbol] | CDZ98521 |
| X₈₃₃₀ | M8 | 262 | 567591 | G | A | [symbol] | CDZ98524 |
| X₈₃₃₁ | M8 | 262 | 567801 | G | A | [symbol] | CDZ98524 |
| X₈₃₃₂ | M8 | 262 | 618198 | G | A | [symbol] | CDZ98538 |
| X₈₃₃₃ | M8 | 262 | 627573 | T | C | [symbol] | CDZ98542 |
| X₈₃₃₄ | M8 | 262 | 627705 | C | T | [symbol] | CDZ98542 |
| X₈₃₃₅ | M8 | 262 | 628911 | G | C | [symbol] | CDZ98543 |
| X₈₃₃₆ | M8 | 262 | 632409 | A | T | [symbol] | CDZ98543 |
| X₈₃₃₇ | M8 | 262 | 632450 | T | G | [symbol] | CDZ98543 |
| X₈₃₃₈ | M8 | 262 | 632476 | T | C | [symbol] | CDZ98543 |
| X₈₃₃₉ | M8 | 262 | 638804 | G | C | [symbol] | CDZ98544 |
| X₈₃₄₀ | M8 | 262 | 638833 | G | T | [symbol] | CDZ98544 |
| X₈₃₄₁ | M8 | 262 | 639962 | A | G | [symbol] | CDZ98544 |
| X₈₃₄₂ | M8 | 262 | 640066 | A | G | [symbol] | CDZ98544 |
| X₈₃₄₃ | M8 | 262 | 644395 | A | G | [symbol] | CDZ98544 |
| X₈₃₄₄ | M8 | 262 | 653920 | A | G | [symbol] | CDZ98545 |
| X₈₃₄₅ | M8 | 262 | 655754 | A | T | [symbol] | CDZ98545 |
| X₈₃₄₆ | M8 | 262 | 655757 | T | C | [symbol] | CDZ98545 |
| X₈₃₄₇ | M8 | 262 | 656323 | C | G | [symbol] | CDZ98545 |
| X₈₃₄₈ | M8 | 262 | 666563 | G | A | [symbol] | CDZ98545 |
| X₈₃₄₉ | M8 | 262 | 668764 | C | T | [symbol] | CDZ98545 |
| X₈₃₅₀ | M8 | 262 | 669984 | C | T | [symbol] | CDZ98547 |
| X₈₃₅₁ | M8 | 262 | 691577 | C | T | [symbol] | CDZ98551 |
| X₈₃₅₂ | M8 | 262 | 771330 | G | A | [symbol] | CDZ98578 |
| X₈₃₅₃ | M8 | 262 | 801565 | G | A | [symbol] | CDZ98589 |
| X₈₃₅₄ | M8 | 262 | 822734 | G | A | [symbol] | CDZ98597 |
| X₈₃₅₅ | M8 | 262 | 825723 | A | G | [symbol] | CDZ98598 |
| X₈₃₅₆ | M8 | 262 | 835874 | C | T | [symbol] | CDZ98601 |
| X₈₃₅₇ | M8 | 262 | 865999 | G | A | [symbol] | CDZ98613 |
| X₈₃₅₈ | M8 | 262 | 886842 | C | T | [symbol] | CDZ98621 |
| X₈₃₅₉ | M8 | 262 | 971713 | G | A | [symbol] | CDZ98649 |
| X₈₃₆₀ | M8 | 262 | 1011054 | G | A | [symbol] | CDZ98659 |
| X₈₃₆₁ | M8 | 262 | 1051389 | C | T | [symbol] | CDZ98670 |
| X₈₃₆₂ | M8 | 262 | 1076581 | G | A | [symbol] | CDZ98679 |
| X₈₃₆₃ | M8 | 262 | 1107286 | C | T | [symbol] | CDZ98689 |
| X₈₃₆₄ | M8 | 262 | 1140198 | C | T | [symbol] | CDZ98701 |
| X₈₃₆₅ | M8 | 262 | 1152058 | C | A | [symbol] | CDZ98703 |
| X₈₃₆₆ | M8 | 262 | 1209662 | G | A | [symbol] | CDZ98723 |
| X₈₃₆₇ | M8 | 262 | 1226072 | C | T | [symbol] | CDZ98730 |
| X₈₃₆₈ | M8 | 262 | 1237021 | C | T | [symbol] | CDZ98735 |
| X₈₃₆₉ | M8 | 262 | 1257961 | C | T | [symbol] | CDZ98739 |
| X₈₃₇₀ | M8 | 262 | 1277604 | G | A | [symbol] | CDZ98747 |
| X₈₃₇₁ | M8 | 262 | 1284428 | G | A | [symbol] | CDZ98748 |
| X₈₃₇₂ | M8 | 262 | 1305361 | G | A | [symbol] | CDZ98753 |
| X₈₃₇₃ | M8 | 262 | 1329643 | C | T | [symbol] | CDZ98760 |
| X₈₃₇₄ | M8 | 262 | 1351116 | A | G | [symbol] | CDZ98766 |
| X₈₃₇₅ | M8 | 262 | 1415479 | C | T | [symbol] | CDZ98789 |
| X₈₃₇₆ | M8 | 262 | 1424293 | G | A | [symbol] | CDZ98791 |
| X₈₃₇₇ | M8 | 262 | 1455677 | G | A | [symbol] | CDZ98804 |
| X₈₃₇₈ | M8 | 262 | 1458863 | C | T | [symbol] | CDZ98807 |
| X₈₃₇₉ | M8 | 262 | 1550311 | C | T | [symbol] | CDZ98836 |
| X₈₃₈₀ | M8 | 262 | 1550456 | C | T | [symbol] | CDZ98837 |
| X₈₃₈₁ | M8 | 262 | 1615762 | G | A | [symbol] | CDZ98855 |
| X₈₃₈₂ | M8 | 262 | 1677620 | G | A | [symbol] | CDZ98873 |
| X₈₃₈₃ | M8 | 262 | 1684375 | G | A | [symbol] | CDZ98876 |
| X₈₃₈₄ | M8 | 262 | 1694625 | C | T | [symbol] | CDZ98878 |
| X₈₃₈₅ | M8 | 262 | 1712334 | G | A | [symbol] | CDZ98886 |
| X₈₃₈₆ | M8 | 262 | 1716385 | G | A | [symbol] | CDZ98887 |
| X₈₃₈₇ | M8 | 262 | 1717611 | C | T | [symbol] | CDZ98888 |
| X₈₃₈₈ | M8 | 266 | 85 | C | T | [symbol] | |
| X₈₃₈₉ | M8 | 266 | 252 | C | T | [symbol] | |
| X₈₃₉₀ | M8 | 267 | 93 | G | A | [symbol] | |
| X₈₃₉₁ | M8 | 267 | 94 | A | G | [symbol] | |
| X₈₃₉₂ | M8 | 267 | 133 | T | C | [symbol] | |
| X₈₃₉₃ | M8 | 267 | 134 | C | T | [symbol] | |
| X₈₃₉₄ | M8 | 267 | 201 | T | C | [symbol] | |
| X₈₃₉₅ | M8 | 267 | 208 | T | C | [symbol] | |

[symbol]

indicates data missing or illegible when filed

TABLE 13

| V | RS | RP | RB | SB | T | GC | CDS | AA Change |
|---|---|---|---|---|---|---|---|---|
| Y₁ | 22 | 1643 | C | T | 2 | cytochrome c oxidase subunit 1 | CED80056 | p.Leu366Phe |
| Y₂ | 22 | 2524 | G | A | 4 | cytochrome c oxidase subunit 1 | CED80056 | |
| Y₃ | 28 | 236 | G | A | 3 | atp synthase subunit 6 | CDZ96150 | |
| Y₄ | 28 | 377 | G | A | 3 | atp synthase subunit 6 | CDZ96150 | |
| Y₅ | 28 | 1506 | C | T | 2 | atp synthase subunit 6 | CDZ96150 | p.Ala227Val |
| Y₆ | 28 | 1677 | C | T | 4 | atp synthase subunit 6 | CDZ96150 | |
| Y₇ | 43 | 464 | G | A | 2 | nadh dehydrogenase subunit 4 | CDZ96151 | p.Thr147Ile |
| Y₈ | 43 | 753 | C | T | 2 | nadh dehydrogenase subunit 4 | CDZ96151 | p.Val51Ile |
| Y₉ | 43 | 1527 | C | T | 3 | nadh dehydrogenase subunit 4 | CDZ96151 | |
| Y₁₀ | 43 | 1748 | G | A | 3 | nadh dehydrogenase subunit 4 | CDZ96151 | |
| Y₁₁ | 43 | 1928 | C | T | 3 | nadh dehydrogenase subunit 4 | CDZ96151 | |
| Y₁₂ | 62 | 875 | G | A | 2 | cytochrome c oxidase subunit 2 | CDZ96152 | p.Thr110Ile |
| Y₁₃ | 102 | 339 | C | T | 4 | NADH dehydrogenase subunits 2, 5, and related proteins | CDZ96153 | |
| Y₁₄ | 102 | 951 | G | A | 4 | NADH dehydrogenase subunits 2, 5, and related proteins | CDZ96153 | |
| Y₁₅ | 102 | 1042 | G | A | 4 | NADH dehydrogenase subunits 2, 5, and related proteins | CDZ96153 | |
| Y₁₆ | 102 | 2334 | G | A | 2 | NADH dehydrogenase subunits 2, 5, and related proteins | CDZ96153 | p.Ser181Phe |
| Y₁₇ | 102 | 2590 | C | T | 2 | NADH dehydrogenase subunits 2, 5, and related proteins | CDZ96153 | p.Ala96Thr |
| Y₁₈ | 102 | 3427 | C | T | 3 | NADH dehydrogenase subunits 2, 5, and related proteins | CDZ96153 | |
| Y₁₉ | 176 | 358 | G | A | 3 | cytochrome b | CED80058 | |
| Y₂₀ | 176 | 1872 | G | A | 4 | cytochrome b | CED80058 | |
| Y₂₁ | 176 | 1980 | G | A | 4 | cytochrome b | CED80058 | |
| Y₂₂ | 179 | 1525 | G | A | 7 | NADH | CDZ96154 | |
| Y₂₃ | 179 | 1780 | C | T | 7 | NADH | CDZ96154 | |
| Y₂₄ | 188 | 815 | C | T | 2 | nadh dehydrogenase subunit 6 | CED80060 | p.Ala79Val |
| Y₂₅ | 188 | 1174 | C | T | 4 | nadh dehydrogenase subunit 6 | CED80060 | |
| Y₂₆ | 188 | 2163 | G | A | 4 | nadh dehydrogenase subunit 6 | CED80060 | |
| Y₂₇ | 188 | 2170 | G | A | 4 | nadh dehydrogenase subunit 6 | CED80060 | |
| Y₂₈ | 247 | 102 | G | A | 4 | cytochrome c oxidase subunit 3 | CED80061 | |
| Y₂₉ | 247 | 989 | C | T | 2 | cytochrome c oxidase subunit 3 | CED80061 | p.Gly104Glu |
| Y₃₀ | 247 | 1053 | G | A | 2 | cytochrome c oxidase subunit 3 | CED80061 | p.Leu83Phe |
| Y₃₁ | 7 | 160 | T | C | 6 | | | |
| Y₃₂ | 8 | 1110 | C | T | 6 | | | |
| Y₃₃ | 11 | 154 | A | C | 6 | | | |
| Y₃₄ | 11 | 158 | C | T | 6 | | | |
| Y₃₅ | 15 | 28 | C | G | 6 | | | |
| Y₃₆ | 24 | 5850 | C | T | 2 | hypothetical protein | CDZ96160 | p.Leu85Phe |
| Y₃₇ | 24 | 180340 | G | A | 3 | V-SNARE | CDZ96216 | |
| Y₃₈ | 24 | 307114 | G | A | 2 | DNA polymerase zeta, catalytic subunit | CDZ96255 | p.Gly717Glu |
| Y₃₉ | 24 | 340663 | T | C | 3 | mRNA splicing factor | CDZ96265 | |
| Y₄₀ | 24 | 420108 | G | A | 3 | Ubiquitin-conjugating enzyme E2-binding protein | CDZ96291 | |
| Y₄₁ | 27 | 445 | C | T | 6 | | | |
| Y₄₂ | 27 | 1012 | A | C | 6 | | | |
| Y₄₃ | 27 | 1013 | T | A | 6 | | | |
| Y₄₄ | 32 | 158 | C | T | 3 | hypothetical protein | CDZ96332 | |
| Y₄₅ | 32 | 215 | C | T | 3 | hypothetical protein | CDZ96332 | |
| Y₄₆ | 32 | 2438 | C | T | 3 | hypothetical protein | CDZ96332 | |
| Y₄₇ | 32 | 2821 | C | T | 7 | hypothetical protein | CDZ96332 | |
| Y₄₈ | 32 | 3067 | C | T | 2 | hypothetical protein | CDZ96332 | p.Ser105Phe |
| Y₄₉ | 32 | 3506 | C | T | 4 | hypothetical protein | CDZ96332 | |
| Y₅₀ | 32 | 3562 | G | A | 4 | hypothetical protein | CDZ96332 | |
| Y₅₁ | 33 | 45488 | C | T | 3 | transcription coactivator | CED82012 | |
| Y₅₂ | 33 | 109397 | G | A | 2 | phosphoenolpyruvate carboxykinase | CED82038 | p.Thr326Ile |
| Y₅₃ | 33 | 260027 | G | A | 3 | hypothetical protein | CED82084 | |
| Y₅₄ | 33 | 274627 | G | A | 8 | Mercaptopyruvate sulfurtransferase/thiosulfate | CED82091 | p.Ala256Thr |
| | | | | | | sulfurtransferase | | |
| Y₅₅ | 33 | 283808 | C | T | 2 | HLH transcription factor EBF/Olf-1 and related | CED82093 | p.Gly36Asp |
| | | | | | | DNA binding proteins | | |
| Y₅₆ | 33 | 341890 | G | A | 2 | Peptidase S9, prolyl oligopeptidase, catalytic domain | CED82113 | p.Pro656Ser |
| Y₅₇ | 33 | 448815 | G | A | 2 | cap1-related protein | CED82144 | p.Val460Ile |
| Y₅₈ | 33 | 453975 | C | T | 2 | C4-type Zn-finger protein | CED82147 | p.Asp703Asn |
| Y₅₉ | 33 | 455451 | C | T | 3 | hypothetical protein | CED82146 | |
| Y₆₀ | 33 | 509127 | C | T | 3 | Endonuclease MUS81 | CED82166 | |
| Y₆₁ | 33 | 693516 | C | T | 2 | p-loop containing nucleoside triphosphate | CED82225 | p.Leu417Phe |
| | | | | | | hydrolase protein | | |
| Y₆₂ | 33 | 1049699 | G | A | 3 | Inorganic phosphate transporter | CED82334 | |
| Y₆₃ | 33 | 1138444 | C | T | 3 | Nuclear AAA ATPase (VCP subfamily) | CED82363 | |
| Y₆₄ | 33 | 1165914 | G | A | 2 | leucine-trna ligase | CED82372 | p.Ser992Phe |
| Y₆₅ | 33 | 1191727 | C | T | 2 | xanthine uracil permease | CED82379 | p.Ser617Asn |
| Y₆₆ | 33 | 1324395 | G | A | 3 | Basic-leucine zipper domain | CED82417 | |
| Y₆₇ | 33 | 1339376 | C | T | 2 | DNA-binding centromere protein B (CENP-B) | CED82424 | p.Thr69Met |
| Y₆₈ | 33 | 1499347 | G | A | 3 | Dimeric dihydrodiol dehydrogenase | CED82472 | |
| Y₆₉ | 33 | 1502644 | G | A | 2 | pre-mrna splicing factor prp1 | CED82474 | p.Pro248Ser |
| Y₇₀ | 33 | 1515369 | G | A | 3 | hypothetical protein | CED82478 | |
| Y₇₁ | 33 | 1544951 | C | G | 6 | hypothetical protein | CED82485 | |
| Y₇₂ | 33 | 1587443 | C | T | 3 | atp-dependent clp protease proteolytic subunit | CED82497 | |
| Y₇₃ | 33 | 1596242 | C | T | 3 | myosin 5 | CED82498 | |
| Y₇₄ | 33 | 1638726 | G | A | 3 | hypothetical protein | CED82513 | |
| Y₇₅ | 33 | 1642659 | G | A | 2 | Uncharacterized MYND Zn-finger protein | CED82515 | p.Ala199Thr |
| Y₇₆ | 33 | 1700000 | G | A | 2 | hypothetical protein | CED82534 | p.Pro888Ser |
| Y₇₇ | 33 | 1938614 | G | A | 2 | Splicing factor 3b, subunit 2 | CED82612 | p.His486Tyr |
| Y₇₈ | 33 | 2010230 | C | T | 2 | OCH1 | CED82636 | p.Thr257Ile |
| Y₇₉ | 33 | 2014235 | C | T | 2 | Putative phosphoinositide phosphatase | CED82637 | p.Val106Ile |
| Y₈₀ | 36 | 320 | G | A | 6 | | | |
| Y₈₁ | 36 | 1056 | G | A | 6 | | | |
| Y₈₂ | 36 | 1295 | C | T | 6 | | | |
| Y₈₃ | 36 | 1461 | A | T | 6 | | | |
| Y₈₄ | 42 | 804 | C | T | 4 | atp synthase subunit mitochondrial | CDZ96333 | |
| Y₈₅ | 42 | 1559 | G | A | 4 | atp synthase subunit mitochondrial | CDZ96333 | |
| Y₈₆ | 50 | 145 | G | A | 6 | | | |
| Y₈₇ | 52 | 63859 | C | T | 2 | kinase-like protein | CED82686 | p.Pro363Ser |
| Y₈₈ | 52 | 144382 | C | T | 3 | hypothetical protein | CED82709 | |
| Y₈₉ | 52 | 151974 | C | T | 2 | Predicted small molecule transporter | CED82712 | p.Gly179Glu |
| Y₉₀ | 52 | 203253 | A | G | 3 | B-block binding subunit of TFIIIC | CED82730 | |
| Y₉₁ | 52 | 207354 | C | T | 8 | B-block binding subunit of TFIIIC | CED82730 | |
| Y₉₂ | 52 | 390095 | G | A | 3 | pre-mrna-splicing factor clf1 | CED82784 | |
| Y₉₃ | 52 | 395374 | G | A | 2 | transcriptional regulator | CED82786 | p.Pro371Ser |
| Y₉₄ | 52 | 398723 | C | T | 3 | transcriptional regulator | CED82786 | |
| Y₉₅ | 52 | 398728 | C | T | 3 | transcriptional regulator | CED82786 | |
| Y₉₆ | 52 | 552099 | G | A | 2 | Zuotin and related molecular chaperones (DnaJ | CED82837 | p.Val134Ile |
| | | | | | | superfamily), contains DNA-binding domains | | |
| Y₉₇ | 52 | 817452 | T | A | 2 | atypical pikk frap protein kinase | CED82928 | p.His270Leu |
| Y₉₈ | 52 | 938650 | G | A | 3 | hypothetical protein | CED82973 | |
| Y₉₉ | 52 | 967316 | A | T | 2 | 1,4-alpha-glucan branching enzyme/starch | CED82984 | p.Glu582Val |
| | | | | | | branching enzyme II | | |
| Y₁₀₀ | 52 | 967701 | T | A | 3 | cyclin-dependent kinase regulatory subunit | CED82983 | |
| Y₁₀₁ | 52 | 1003899 | G | A | 2 | heat shock protein 60 ame | CED83001 | p.Gly126Asp |
| Y₁₀₂ | 52 | 1151398 | G | A | 3 | FOG | CED83046 | |
| Y₁₀₃ | 52 | 1158103 | C | T | 3 | Density-regulated protein related to translation | CED83051 | |
| | | | | | | initiation factor 1 (elF-1/SUI1) | | |
| Y₁₀₄ | 52 | 1219894 | C | T | 3 | probable cdc12-septin | CED83071 | |
| Y₁₀₅ | 52 | 1363357 | G | A | 3 | mrg-domain-containing protein | CED83122 | |
| Y₁₀₆ | 52 | 1399439 | G | A | 2 | mitochondrial cytochrome b2- | CED83134 | p.Arg244Gln |
| Y₁₀₇ | 52 | 1526796 | G | A | 2 | eukaryotic translation initiation factor 2 subunit alpha | CED83181 | p.Gly134Glu |
| Y₁₀₈ | 52 | 1583655 | C | T | 2 | Predicted Zn-finger protein | CED83206 | p.Pro285Leu |
| Y₁₀₉ | 52 | 1708900 | A | G | 3 | dna mismatch repair protein | CED83249 | |
| Y₁₁₀ | 52 | 1716210 | C | T | 3 | atpase | CED83250 | |
| Y₁₁₁ | 52 | 1793370 | G | A | 3 | RNA polymerase II, subunit POLR2C/RPB3 | CED83275 | |
| Y₁₁₂ | 52 | 1807309 | G | A | 3 | Protein of unknown function DUF1264 | CED83283 | |
| Y₁₁₃ | 52 | 1871310 | C | T | 2 | Uncharacterized conserved protein | CED83304 | p.Ala574Val |
| Y₁₁₄ | 52 | 1893352 | G | A | 2 | atp-dependent metallopeptidase hfl | CED83313 | p.Pro375Leu |
| Y₁₁₅ | 52 | 1915927 | G | A | 3 | hypothetical protein | CED83315 | |
| Y₁₁₆ | 52 | 1987096 | G | A | 4 | Sexual differentiation process protein ISP4 | CED83341 | |
| Y₁₁₇ | 52 | 2050245 | G | A | 3 | Extracellular protein SEL-1 and related proteins | CED83359 | |
| Y₁₁₈ | 52 | 2120665 | C | T | 4 | hypothetical protein | CED83376 | |
| Y₁₁₉ | 52 | 2162878 | G | A | 2 | Nucleic acid-binding, OB-fold | CED83392 | p.Asp942Asn |
| Y₁₂₀ | 52 | 2258192 | C | T | 2 | hypothetical protein | CED83422 | p.Pro126Ser |
| Y₁₂₁ | 52 | 2265219 | C | T | 2 | hypothetical protein | CED83425 | p.Glu74Lys |
| Y₁₂₂ | 52 | 2266378 | C | T | 3 | dead-domain-containing protein | CED83423 | |
| Y₁₂₃ | 52 | 2327266 | C | T | 3 | 60s ribosomal protein I21 | CED83445 | |
| Y₁₂₄ | 52 | 2373120 | C | T | 2 | Protein phosphatase, regulatory subunit | CED83456 | p.Gly789Glu |
| | | | | | | PPP1R3C/D | | |
| Y₁₂₅ | 52 | 2400778 | C | T | 4 | atp-dependent rna helicase dhx8 | CED83463 | |
| Y₁₂₆ | 52 | 2543220 | C | T | 3 | Protein of unknown function DUF3468 | CED83509 | |
| Y₁₂₇ | 53 | 26291 | G | A | 2 | Uncharacterised protein family UPF0390 | CDZ96348 | p.Ser75Asn |
| Y₁₂₈ | 54 | 106086 | G | A | 3 | FOG | CDZ96416 | |
| Y₁₂₉ | 54 | 194230 | G | A | 2 | RmlC-like jelly roll fold | CDZ96446 | p.Pro179Ser |
| Y₁₃₀ | 54 | 206494 | G | A | 2 | white collar 1 protein | CDZ96450 | p.Asp88Asn |
| Y₁₃₁ | 54 | 370320 | G | A | 2 | UV radiation resistance associated protein | CDZ96499 | p.Thr57Ile |
| Y₁₃₂ | 54 | 391223 | A | G | 2 | SAP family cell cycle dependent phosphatase- | CDZ96509 | p.Asp477Gly |
| | | | | | | associated protein | | |
| Y₁₃₃ | 54 | 427542 | C | T | 2 | Exosomal 3′-5′ exoribonuclease complex, subunit | CDZ96520 | p.Ser51Asn |
| | | | | | | Rrp44/Dis3 | | |
| Y₁₃₄ | 54 | 431851 | C | T | 3 | Exosomal 3′-5′ exoribonuclease complex, subunit | CDZ96520 | |
| | | | | | | Rrp44/Dis3 | | |
| Y₁₃₅ | 54 | 478527 | C | T | 2 | abc transporter | CDZ96537 | p.Val1423Ile |
| Y₁₃₆ | 54 | 555986 | G | A | 2 | hypothetical protein | CDZ96563 | p.Pro8Ser |
| Y₁₃₇ | 54 | 733193 | T | C | start_lost | hypothetical protein | CDZ96619 | p.Met1? |
| Y₁₃₈ | 67 | 111 | G | A | 6 | | | |
| Y₁₃₉ | 69 | 29006 | G | A | 3 | hypothetical protein | CED83534 | |
| Y₁₄₀ | 69 | 201005 | G | A | 3 | hypothetical protein | CED83595 | |
| Y₁₄₁ | 69 | 305997 | C | T | 2 | Peptidase M28 | CED83631 | p.Leu341Phe |
| Y₁₄₂ | 69 | 384418 | G | A | 2 | Delta 9 fatty acid desaturase | CED83656 | p.Ala149Thr |
| Y₁₄₃ | 69 | 415659 | C | T | 2 | microtubule binding protein | CED83665 | p.Asp305Asn |
| Y₁₄₄ | 69 | 467686 | C | T | 2 | dna replication factor large subunit | CED83681 | p.Asp244Asn |
| Y₁₄₅ | 69 | 585845 | G | A | 2 | beta and beta-prime subunits of dna dependent | CED83722 | p.Gly1099Ser |
| | | | | | | rna-polymerase | | |
| Y₁₄₆ | 69 | 631511 | G | A | 2 | aspartate-semialdehyde dehydrogenase | CED83742 | p.Ala15Thr |
| Y₁₄₇ | 69 | 637299 | T | C | 2 | hypothetical protein | CED83744 | p.Thr41Ala |
| Y₁₄₈ | 69 | 675579 | G | A | 3 | hypothetical protein | CED83760 | |
| Y₁₄₉ | 69 | 676581 | G | A | 2 | hypothetical protein | CED83760 | p.Ala2Thr |
| Y₁₅₀ | 69 | 676853 | G | A | 3 | Metalloexopeptidases | CED83759 | |
| Y₁₅₁ | 69 | 678406 | C | T | 3 | Metalloexopeptidases | CED83759 | |
| Y₁₅₂ | 69 | 865820 | C | T | 2 | acyl-oxidase | CED83814 | p.Leu1253Phe |
| Y₁₅₃ | 69 | 869706 | G | A | 8 | Predicted E3 ubiquitin ligase | CED83815 | |
| Y₁₅₄ | 69 | 894462 | C | T | 3 | TPR repeat-containing protein | CED83821 | |
| Y₁₅₅ | 69 | 971219 | C | T | 3 | probable peroxisomal half abc transporter | CED83847 | |
| Y₁₅₆ | 69 | 988364 | G | A | 2 | hypothetical protein | CED83857 | p.Gly911Glu |
| Y₁₅₇ | 69 | 1102164 | C | T | 2 | nucleosome assembly protein | CED83897 | p.Glu89Lys |
| Y₁₅₈ | 69 | 1218409 | G | A | 3 | FOG | CED83936 | |
| Y₁₅₉ | 69 | 1260087 | G | A | 3 | mRNA cleavage and polyadenylation factor II | CED83949 | |
| | | | | | | complex, subunit CFT2 (CPSF subunit) | | |
| Y₁₆₀ | 69 | 1342909 | G | A | 3 | Inositol polyphosphate multikinase, component | CED83975 | |
| | | | | | | of the ARGR transcription regulatory complex | | |
| Y₁₆₁ | 69 | 1411421 | C | T | 3 | hypothetical protein | CED83999 | |
| Y₁₆₂ | 69 | 1652288 | C | T | 3 | atp-dependent rna helicase dbp5 | CED84077 | |
| Y₁₆₃ | 69 | 1714728 | G | A | 3 | hypothetical protein | CED84097 | |
| Y₁₆₄ | 69 | 1790823 | C | T | 2 | Protein of unknown function DUF1183, TMEM66 | CED84125 | p.Ala253Val |
| Y₁₆₅ | 69 | 1792597 | C | T | 2 | SMAD/FHA domain | CED84126 | p.Ala258Val |
| Y₁₆₆ | 69 | 1800584 | G | A | 3 | Uncharacterized conserved protein | CED84129 | |
| Y₁₆₇ | 69 | 1854739 | G | A | 3 | RNA polymerase II transcription | CED84145 | |
| | | | | | | initiation/nucleotide excision repair factor TFIIH, | | |
| | | | | | | subunit TFB2 | | |
| Y₁₆₈ | 69 | 1924621 | C | T | 3 | hypothetical protein | CED84172 | |
| Y₁₆₉ | 69 | 1968848 | A | G | 3 | Immunoglobulin-like fold | CED84189 | |
| Y₁₇₀ | 69 | 2001469 | C | T | 3 | pre-mrna splicing factor | CED84197 | |
| Y₁₇₁ | 69 | 2255001 | G | A | 2 | hypothetical protein | CED84282 | p.Ala239Val |
| Y₁₇₂ | 69 | 2438727 | C | T | 3 | g-protein alpha-subunit | CED84343 | |
| Y₁₇₃ | 69 | 2483433 | G | A | 3 | F-box protein containing LRR | CED84352 | |
| Y₁₇₄ | 72 | 584 | G | A | 6 | | | |
| Y₁₇₅ | 72 | 856 | C | T | 6 | | | |
| Y₁₇₆ | 72 | 1163 | C | T | 6 | | | |
| Y₁₇₇ | 77 | 84022 | G | A | 8 | DRIM (Down-regulated in metastasis)-like proteins | CDZ96659 | |
| Y₁₇₈ | 78 | 6333 | G | A | 8 | acid phosphatase | CED84360 | |
| Y₁₇₉ | 78 | 238854 | C | T | 3 | Extracellular matrix glycoprotein Laminin | CED84430 | |
| | | | | | | subunits alpha and gamma | | |
| Y₁₈₀ | 78 | 299644 | G | A | 3 | hypothetical protein | CED84443 | |
| Y₁₈₁ | 78 | 444292 | C | T | 2 | gtp-binding protein ypt1 | CED84490 | p.Ala156Val |
| Y₁₈₂ | 78 | 666005 | C | T | 3 | Protein involved in DNA repair | CED84562 | |
| Y₁₈₃ | 78 | 763771 | G | A | 3 | mrna (guanine-n7-)-methyltransferase | CED84600 | |
| Y₁₈₄ | 78 | 774252 | G | A | 2 | ataxia telangiectasia mutated | CED84602 | p.Val2842Ile |
| Y₁₈₅ | 78 | 952815 | G | A | 2 | Ornithine decarboxylase antizyme | CED84662 | p.Gly246Glu |
| Y₁₈₆ | 78 | 952860 | G | A | 2 | Ornithine decarboxylase antizyme | CED84662 | p.Gly261Glu |
| Y₁₈₇ | 78 | 1023381 | G | A | 2 | Aldo/keto reductase family proteins | CED84688 | p.Val43Ile |
| Y₁₈₈ | 78 | 1302286 | C | T | 3 | Spermidine/spermine synthases family | CED84782 | |
| Y₁₈₉ | 78 | 1317124 | G | A | 3 | hypothetical protein | CED84787 | |
| Y₁₉₀ | 78 | 1344640 | C | T | 3 | Predicted membrane protein | CED84795 | |
| Y₁₉₁ | 78 | 1426722 | C | T | 2 | high-affinity cell membrane calcium channel | CED84829 | p.Val866Ile |
| Y₁₉₂ | 78 | 1568326 | C | T | 2 | WD40/YVTN repeat-like-containing domain | CED84868 | p.Val1033Ile |
| Y₁₉₃ | 78 | 1642170 | G | A | 3 | hypothetical protein | CED84889 | |
| Y₁₉₄ | 78 | 1660408 | G | A | 2 | golgi apparatus membrane protein tvp23 | CED84897 | p.Thr539Ile |
| Y₁₉₅ | 78 | 1694684 | C | T | 2 | Conserved hypothetical protein CHP02453 | CED84912 | p.Asp239Asn |
| Y₁₉₆ | 79 | 57804 | G | A | 2 | AAA-type ATPase | CDZ96687 | p.Glu1838Lys |
| Y₁₉₇ | 79 | 132683 | C | T | 2 | Immunoglobulin-like fold | CDZ96713 | p.Ser156Asn |
| Y₁₉₈ | 79 | 307021 | C | T | 3 | HSP20-like chaperone | CDZ96777 | |
| Y₁₉₉ | 79 | 346823 | G | A | 2 | Pantothenate kinase PanK and related proteins | CDZ96789 | p.Gly590Arg |
| Y₂₀₀ | 79 | 492523 | C | T | 3 | hypothetical protein | CDZ96842 | |
| Y₂₀₁ | 79 | 575411 | C | T | 3 | Mediator complex, subunit Med4 | CDZ96868 | |
| Y₂₀₂ | 79 | 617057 | C | T | 2 | M13 family peptidase | CDZ96882 | p.Gly670Asp |
| Y₂₀₃ | 79 | 679140 | C | T | 2 | ca-transporting atpase | CDZ96904 | p.Met334Ile |
| Y₂₀₄ | 79 | 696174 | G | A | 4 | | CDZ96911 | |
| Y₂₀₅ | 79 | 747717 | A | G | 3 | Predicted histone tail methylase containing | CDZ96929 | |
| | | | | | | SET domain | | |
| Y₂₀₆ | 79 | 759589 | G | A | 4 | FOG | CDZ96932 | |
| Y₂₀₇ | 79 | 776112 | A | G | 4 | ribonuclease iii | CDZ96934 | |
| Y₂₀₈ | 79 | 1123288 | C | T | 2 | Zinc finger, RING-type | CDZ97049 | p.Thr364Ile |
| Y₂₀₉ | 79 | 1140763 | G | A | 3 | 40s ribosomal protein s15 | CDZ97053 | |
| Y₂₁₀ | 79 | 1229129 | G | A | 2 | nuclear export receptor crm1 | CDZ97086 | p.Leu253Phe |
| Y₂₁₁ | 79 | 1231740 | C | T | 2 | voltage-gated chloride channel | CDZ97087 | p.Arg715His |
| Y₂₁₂ | 79 | 1336766 | C | T | 4 | phosphatidylinositol 4-kinase | CDZ97121 | |
| Y₂₁₃ | 79 | 1430954 | G | A | 2 | hypothetical protein | CDZ97152 | p.Pro1037Ser |
| Y₂₁₄ | 79 | 1540205 | C | T | 2 | Geranylgeranyl pyrophosphate synthase | CDZ97186 | p.Met237Ile |
| Y₂₁₅ | 79 | 1674448 | G | A | 4 | Membrane coat complex Retromer, | CDZ97228 | |
| | | | | | | subunit VPS5/SNX1, Sorting nexins, and | | |
| | | | | | | related PX domain-containing proteins | | |
| Y₂₁₆ | 79 | 1797660 | C | T | 3 | Iron permease FTR1 | CDZ97265 | |
| Y₂₁₇ | 79 | 1797676 | G | A | 3 | Iron permease FTR1 | CDZ97265 | |
| Y₂₁₈ | 79 | 1842448 | G | A | 2 | t-complex protein alpha subunit (tcp-1-alpha) | CDZ97282 | p.Ser212Asn |
| Y₂₁₉ | 79 | 1855037 | A | G | 3 | hypothetical protein | CDZ97285 | |
| Y₂₂₀ | 79 | 2013361 | C | T | 8 | Predicted membrane protein | CDZ97344 | |
| Y₂₂₁ | 79 | 2027861 | C | T | 8 | Predicted short chain-type dehydrogenase | CDZ97350 | |
| Y₂₂₂ | 79 | 2121041 | G | A | 3 | Polyadenylation factor I complex, subunit, | CDZ97380 | |
| | | | | | | Yth1 (CPSF subunit) | | |
| Y₂₂₃ | 79 | 2152573 | C | T | 2 | Predicted lipase | CDZ97392 | p.Gly230Asp |
| Y₂₂₄ | 79 | 2188410 | G | A | 2 | RNA polymerase III, subunit C34 | CDZ97405 | p.Glu121Lys |
| Y₂₂₅ | 85 | 512 | A | G | 6 | | | |
| Y₂₂₆ | 85 | 516 | G | A | 6 | | | |
| Y₂₂₇ | 88 | 119 | C | T | 3 | hypothetical protein | CDZ97464 | |
| Y₂₂₈ | 88 | 196 | G | A | 3 | hypothetical protein | CDZ97464 | |
| Y₂₂₉ | 88 | 898 | C | T | 4 | hypothetical protein | CDZ97464 | |
| Y₂₃₀ | 99 | 245 | G | A | 6 | | | |
| Y₂₃₁ | 109 | 396 | T | C | 6 | | | |
| Y₂₃₂ | 118 | 396 | C | T | 2 | hypothetical protein | CDZ97478 | p.Pro41Ser |
| Y₂₃₃ | 118 | 870 | G | A | 4 | hypothetical protein | CDZ97478 | |
| Y₂₃₄ | 118 | 1491 | G | A | 4 | hypothetical protein | CDZ97478 | |
| Y₂₃₅ | 124 | 72117 | G | A | 2 | p-loop containing nucleoside triphosphate | CDZ97501 | p.Gly510Asp |
| | | | | | | hydrolase protein | | |
| Y₂₃₆ | 124 | 156052 | G | A | 2 | cytoplasm protein | CDZ97524 | p.Ala504Thr |
| Y₂₃₇ | 125 | 35864 | T | C | 3 | Plant ascorbate peroxidase | CDZ97573 | |
| Y₂₃₈ | 129 | 299 | G | A | 3 | hypothetical protein | CDZ97577 | |
| Y₂₃₉ | 129 | 433 | G | A | 3 | hypothetical protein | CDZ97577 | |
| Y₂₄₀ | 129 | 856 | G | A | 2 | hypothetical protein | CDZ97577 | p.Gly103Glu |
| Y₂₄₁ | 130 | 674 | C | T | 4 | hypothetical protein | CDZ97578 | |
| Y₂₄₂ | 141 | 15764 | C | T | 2 | lysine-trna ligase | CDZ97587 | p.Gly589Asp |
| Y₂₄₃ | 162 | 223407 | C | T | 2 | Protein of unknown function DUF2419 | CDZ97674 | p.Gly204Glu |
| Y₂₄₄ | 162 | 321070 | C | T | 4 | retrotransposon ty1-copia subclass | CDZ97703 | |
| Y₂₄₅ | 162 | 321073 | A | G | 4 | retrotransposon ty1-copia subclass | CDZ97703 | |
| Y₂₄₆ | 162 | 360320 | G | A | 2 | mRNA splicing factor | CDZ97714 | p.Ala393Val |
| Y₂₄₇ | 162 | 424391 | C | T | 3 | gtp-binding protein | CDZ97740 | |
| Y₂₄₈ | 162 | 444123 | T | C | 2 | ring finger protein | CDZ97748 | p.Asp157Gly |
| Y₂₄₉ | 162 | 467207 | C | T | 3 | cytochrome b5 | CDZ97755 | |
| Y₂₅₀ | 162 | 491906 | G | A | 3 | hypothetical protein | CDZ97764 | |
| Y₂₅₁ | 162 | 501886 | C | T | 8 | Tetraspanin/Peripherin | CDZ97769 | |
| Y₂₅₂ | 162 | 632311 | G | A | 2 | snf2 family amino-terminal protein | CDZ97814 | p.Gly88Asp |
| Y₂₅₃ | 162 | 633321 | G | A | 2 | snf2 family amino-terminal protein | CDZ97814 | p.Gly304Ser |
| Y₂₅₄ | 162 | 640005 | C | T | 2 | arginyl-trna synthetase | CDZ97815 | p.Arg318Lys |
| Y₂₅₅ | 162 | 808830 | G | A | 3 | dihydropteroate synthase | CDZ97869 | |
| Y₂₅₆ | 162 | 871911 | C | T | 8 | Predicted Zn-finger protein | CDZ97890 | |
| Y₂₅₇ | 189 | 126573 | G | A | 3 | START-like domain | CDZ97996 | |
| Y₂₅₈ | 189 | 323680 | C | T | 2 | hypothetical protein | CDZ98063 | p.Ala390Thr |
| Y₂₅₉ | 189 | 371247 | G | A | 5 | WD40 repeat-containing protein | CDZ98078 | p.Arg144* |
| Y₂₆₀ | 197 | 624 | C | T | 3 | hypothetical protein | CDZ98111 | |
| Y₂₆₁ | 198 | 238 | C | T | 6 | | | |
| Y₂₆₂ | 198 | 265 | C | T | 6 | | | |
| Y₂₆₃ | 199 | 688 | C | T | 6 | | | |
| Y₂₆₄ | 199 | 911 | C | T | 6 | | | |
| Y₂₆₅ | 199 | 971 | C | T | 6 | | | |
| Y₂₆₆ | 199 | 1218 | C | T | 6 | | | |
| Y₂₆₇ | 202 | 171 | C | T | 6 | | | |
| Y₂₆₈ | 205 | 360 | C | T | 3 | hypothetical protein | CDZ98112 | |
| Y₂₆₉ | 205 | 981 | C | T | 4 | hypothetical protein | CDZ98112 | |
| Y₂₇₀ | 208 | 117 | G | A | 6 | | | |
| Y₂₇₁ | 208 | 476 | G | A | 6 | | | |
| Y₂₇₂ | 208 | 616 | G | A | 6 | | | |
| Y₂₇₃ | 208 | 935 | G | A | 6 | | | |
| Y₂₇₄ | 209 | 312 | C | T | 8 | hypothetical protein | CDZ98113 | |
| Y₂₇₅ | 225 | 317 | C | T | 6 | | | |
| Y₂₇₆ | 227 | 202 | G | A | 6 | | | |
| Y₂₇₇ | 232 | 340 | G | A | 3 | hypothetical protein | CDZ98119 | |
| Y₂₇₈ | 232 | 1521 | C | T | 2 | hypothetical protein | CDZ98119 | p.Pro84Leu |
| Y₂₇₉ | 232 | 1662 | G | A | 2 | hypothetical protein | CDZ98119 | p.Ser131Asn |
| Y₂₈₀ | 232 | 1761 | C | T | 2 | hypothetical protein | CDZ98119 | p.Ser140Phe |
| Y₂₈₁ | 232 | 2027 | C | T | 4 | hypothetical protein | CDZ98119 | |
| Y₂₈₂ | 232 | 3060 | C | T | 2 | hypothetical protein | CDZ98120 | p.Gly115Glu |
| Y₂₈₃ | 232 | 3284 | C | T | 4 | hypothetical protein | CDZ98119 | |
| Y₂₈₄ | 232 | 4437 | G | A | 2 | hypothetical protein | CDZ98121 | p.Ser21Phe |
| Y₂₈₅ | 232 | 4561 | C | T | 3 | hypothetical protein | CDZ98120 | |
| Y₂₈₆ | 242 | 7334 | C | T | 2 | alanine-trna ligase | CDZ98125 | p.Asp781Asn |
| Y₂₈₇ | 242 | 41710 | G | A | 3 | Protein kinase essential for the initiation of | CDZ98131 | |
| | | | | | | DNA replication | | |
| Y₂₈₈ | 242 | 50262 | G | A | 8 | palp-domain-containing protein | CDZ98135 | |
| Y₂₈₉ | 242 | 88037 | G | A | 2 | hypothetical protein | CDZ98147 | p.Leu39Phe |
| Y₂₉₀ | 242 | 107587 | G | A | 4 | arginase deacetylase | CDZ98153 | |
| Y₂₉₁ | 242 | 213986 | G | A | 8 | atp-nad kinase | CDZ98185 | p.Asp654Asn |
| Y₂₉₂ | 242 | 227271 | C | T | 3 | hypothetical protein | CDZ98191 | |
| Y₂₉₃ | 242 | 233088 | G | A | 2 | fumarate reductase | CDZ98193 | p.Gly11Glu |
| Y₂₉₄ | 242 | 310353 | G | A | 2 | hypothetical protein | CDZ98216 | p.Ser76Phe |
| Y₂₉₅ | 242 | 404132 | G | A | 3 | hypothetical protein | CDZ98248 | |
| Y₂₉₆ | 242 | 412676 | G | A | 3 | peptidase c1b bleomycin hydrolase | CDZ98249 | |
| Y₂₉₇ | 242 | 485936 | G | A | 4 | Cytochrome c oxidase, subunit IV/COX5b | CDZ98270 | |
| Y₂₉₈ | 242 | 492389 | G | A | 2 | 1-aminocyclopropane-1-carboxylate | CDZ98274 | p.Glu201Lys |
| | | | | | | synthase, and related proteins | | |
| Y₂₉₉ | 242 | 514780 | C | T | 2 | endosomal p24a protein | CDZ98282 | p.Val585Met |
| Y₃₀₀ | 242 | 542663 | G | A | 3 | Sirtuin 4 and related class Il sirtuins (SIR2 family) | CDZ98290 | |
| Y₃₀₁ | 242 | 559999 | G | A | 2 | Protein-tyrosine/Dual specificity phosphatase | CDZ98297 | p.Glu29Lys |
| Y₃₀₂ | 242 | 602456 | G | A | 2 | calcineurin responsive transcription factor prz1 | CDZ98308 | p.Asp190Asn |
| Y₃₀₃ | 242 | 602531 | G | A | 2 | calcineurin responsive transcription factor prz1 | CDZ98308 | p.Ala215Thr |
| Y₃₀₄ | 242 | 604704 | C | T | 2 | hypothetical protein | CDZ98309 | p.Pro155Leu |
| Y₃₀₅ | 249 | 72016 | C | T | 3 | hypothetical protein | CED84950 | |
| Y₃₀₆ | 249 | 157550 | C | T | 8 | acyl-n-acyltransferase | CED84983 | |
| Y₃₀₇ | 249 | 191304 | C | T | 2 | Maintenance of telomere capping protein 1, Mtc1 | CED84988 | p.Gly64Ser |
| Y₃₀₈ | 249 | 272075 | C | T | 8 | beta-1,6-N-acetylglucosaminyltransferase, | CED85013 | |
| | | | | | | contains WSC domain | | |
| Y₃₀₉ | 249 | 305975 | G | A | 2 | Zn(2)-C6 fungal-type DNA-binding domain | CED85023 | p.Val135Ile |
| Y₃₁₀ | 249 | 485782 | C | T | 3 | Nuclear pore complex, Nup155 component | CED85089 | |
| | | | | | | (D Nup154, sc Nup157/Nup170) | | |
| Y₃₁₁ | 249 | 501310 | C | T | 3 | hypothetical protein | CED85093 | |
| Y₃₁₂ | 249 | 532943 | A | G | 3 | Zn(2)-C6 fungal-type DNA-binding domain | CED85099 | |
| Y₃₁₃ | 249 | 598579 | C | T | 2 | hypothetical protein | CED85125 | p.Val310Ile |
| Y₃₁₄ | 249 | 626627 | G | A | 2 | hypothetical protein | CED85132 | p.Asp723Asn |
| Y₃₁₅ | 249 | 660408 | G | A | 2 | Zn(2)-C6 fungal-type DNA-binding domain | CED85144 | p.Pro300Ser |
| Y₃₁₆ | 249 | 854220 | G | A | 2 | E3 ubiquitin protein ligase | CED85201 | p.Leu266Phe |
| Y₃₁₇ | 249 | 911274 | C | T | 2 | Putative transmembrane protein | CED85218 | p.Thr1003Ile |
| Y₃₁₈ | 249 | 932606 | C | T | 3 | hypothetical protein | CED85223 | |
| Y₃₁₉ | 249 | 945434 | C | T | 3 | Major facilitator superfamily domain, | CED85227 | |
| | | | | | | general substrate transporter | | |
| Y₃₂₀ | 249 | 945727 | C | T | 3 | Major facilitator superfamily domain, | CED85227 | |
| | | | | | | general substrate transporter | | |
| Y₃₂₁ | 249 | 977170 | C | T | 2 | protein kinase | CED85242 | p.Thr32Ile |
| Y₃₂₂ | 249 | 989520 | C | T | 3 | Glucosyltransferase-Alg8p | CED85243 | |
| Y₃₂₃ | 249 | 1014098 | C | T | 2 | Golgi-associated protein/Nedd4 WW | CED85252 | p.Val612Ile |
| | | | | | | domain-binding protein | | |
| Y₃₂₄ | 249 | 1144309 | G | A | 3 | Origin recognition complex, subunit 1, and | CED85291 | |
| | | | | | | related proteins | | |
| Y₃₂₅ | 249 | 1230068 | C | T | 2 | cysteine proteinase | CED85319 | p.Ala803Thr |
| Y₃₂₆ | 249 | 1619036 | G | A | 2 | related to c2h2 zinc finger protein flbc | CED85450 | p.Asp967Asn |
| Y₃₂₇ | 249 | 1726777 | C | T | 3 | C-14 reductase | CED85474 | |
| Y₃₂₈ | 249 | 1803575 | C | T | 2 | Hydroxymethylglutaryl-CoA reductase | CED85502 | p.Pro144Ser |
| Y₃₂₉ | 249 | 1988505 | C | T | 3 | Zn(2)-C6 fungal-type DNA-binding domain | CED85560 | |
| Y₃₃₀ | 249 | 2015272 | G | A | 2 | RNA polymerase II transcription | CED85567 | p.Arg120Lys |
| | | | | | | initiation/nucleotide excision repair factor | | |
| | | | | | | TFIIH, subunit TFB4 | | |
| Y₃₃₁ | 249 | 2198205 | G | A | 8 | Zn(2)-C6 fungal-type DNA-binding domain | CED85628 | |
| Y₃₃₂ | 252 | 512 | C | T | 6 | | | |
| Y₃₃₃ | 252 | 524 | C | T | 6 | | | |
| Y₃₃₄ | 260 | 156 | G | A | 6 | | | |
| Y₃₃₅ | 262 | 30347 | G | A | 2 | Microfibrillar-associated protein MFAP1 | CDZ98338 | p.Glu44Lys |
| Y₃₃₆ | 262 | 30359 | G | A | 2 | Microfibrillar-associated protein MFAP1 | CDZ98338 | p.Gly48Arg |
| Y₃₃₇ | 262 | 148151 | C | T | 3 | Tetratricopeptide-like helical | CDZ98375 | |
| Y₃₃₈ | 262 | 164438 | G | A | 2 | hypothetical protein | CDZ98383 | p.Gly276Glu |
| Y₃₃₉ | 262 | 201531 | G | A | 8 | snf2-family atp dependent chromatin | CDZ98396 | p.Arg1238Gln |
| | | | | | | remodeling factor snf21 | | |
| Y₃₄₀ | 262 | 252708 | G | A | 8 | arsenical pump-driving atpase | CDZ98414 | p.Gly39Asp |
| Y₃₄₁ | 262 | 380519 | C | T | 4 | guanylate kinase | CDZ98457 | |
| Y₃₄₂ | 262 | 397539 | G | A | 3 | fatty acid-2 hydroxylase | CDZ98463 | |
| Y₃₄₃ | 262 | 420102 | G | A | 2 | Sucrose transporter and related proteins | CDZ98468 | p.Thr146Ile |
| Y₃₄₄ | 262 | 446956 | C | T | 2 | RNA polymerase I termination factor, Myb | CDZ98477 | p.Ala859Val |
| | | | | | | superfamily | | |
| Y₃₄₅ | 262 | 557280 | G | A | 2 | Uncharacterized conserved protein | CDZ98518 | p.Gly2213Arg |
| Y₃₄₆ | 262 | 560713 | G | A | 8 | Uncharacterized conserved protein | CDZ98519 | |
| Y₃₄₇ | 262 | 562795 | C | T | 2 | Ferredoxin/adrenodoxin reductase | CDZ98521 | p.Gly327Asp |
| Y₃₄₈ | 262 | 567591 | G | A | 2 | wd40 repeat-like protein | CDZ98524 | p.Pro364Ser |
| Y₃₄₉ | 262 | 567801 | G | A | 2 | wd40 repeat-like protein | CDZ98524 | p.Pro294Ser |
| Y₃₅₀ | 262 | 618198 | G | A | 3 | Hsp90 co-chaperone CNS1 (contains TPR repeats) | CDZ98538 | |
| Y₃₅₁ | 262 | 639962 | A | G | 4 | hypothetical protein | CDZ98544 | |
| Y₃₅₂ | 262 | 656323 | C | G | 4 | RhoGEF GTPase | CDZ98545 | |
| Y₃₅₃ | 262 | 668764 | C | T | 3 | RhoGEF GTPase | CDZ98545 | |
| Y₃₅₄ | 262 | 691577 | C | T | 2 | Protein involved in vacuole import and degradation | CDZ98551 | p.Gly28Arg |
| Y₃₅₅ | 262 | 822734 | G | A | 2 | hypothetical protein | CDZ98597 | p.Gly184Asp |
| Y₃₅₆ | 262 | 835874 | C | T | 3 | FOG | CDZ98601 | |
| Y₃₅₇ | 262 | 865999 | G | A | 3 | Mitochondrial Fe2 transporter MMT1 and | CDZ98613 | |
| | | | | | | related transporters (cation diffusion | | |
| | | | | | | facilitator superfamily) | | |
| Y₃₅₈ | 262 | 971713 | G | A | 3 | Glycoside hydrolase, superfamily | CDZ98649 | |
| Y₃₅₉ | 262 | 1209662 | G | A | 2 | CLASP N-terminal domain | CDZ98723 | p.Leu48Phe |
| Y₃₆₀ | 262 | 1226072 | C | T | 2 | hypothetical protein | CDZ98730 | p.Ala131Val |
| Y₃₆₁ | 262 | 1305361 | G | A | 3 | Extracellular matrix glycoprotein Laminin | CDZ98753 | |
| | | | | | | subunits alpha and gamma | | |
| Y₃₆₂ | 262 | 1424293 | G | A | 2 | Pinin/SDK/MemA protein | CDZ98791 | p.Leu131Phe |
| Y₃₆₃ | 262 | 1455677 | G | A | 5 | G protein-coupled receptor, rhodopsin-like | CDZ98804 | p.Trp395* |
| Y₃₆₄ | 262 | 1550311 | C | T | 3 | Uncharacterized conserved protein | CDZ98836 | |
| Y₃₆₅ | 262 | 1615762 | G | A | 2 | histone deacetylase clr6 | CDZ98855 | p.Gly615Glu |
| Y₃₆₆ | 262 | 1677620 | G | A | 3 | Methylase | CDZ98873 | |
| Y₃₆₇ | 262 | 1694625 | C | T | 3 | RAB proteins geranylgeranyltransferase | CDZ98878 | |
| | | | | | | component A (RAB escort protein) | | |
| Y₃₆₈ | 266 | 252 | C | T | 6 | | | |

TABLE 14

| Var- | | | Refer- | | | | | | |
|---|---|---|---|---|---|---|---|---|---|
| ia- | Reference | Reference | ence | Strain | | | | | Amino Acid |
| tion | Scaffold | Position | Base | Base | Change Type | Impact | Gene Classification | CDS | Change |
| Z₁ | Scaffold_262 | 562795 | C | T | missense_variant | MODERATE | Ferredoxin/ | CDZ98521 | p.Gly327Asp |
| | | | | | | | adrenodoxin | | |
| | | | | | | | reductase | | |
| Z₂ | Scaffold_52 | 1399439 | G | A | missense_variant | MODERATE | Mitochondrial | CED83134 | p.Arg244Gln |
| | | | | | | | cytochrome b2- | | |
| Z₃ | Scaffold_176 | 358 | G | A | upstream_gene_variant | MODIFIER | Cytochrome b | CED80058 | |
| Z₄ | Scaffold_22 | 1643 | C | T | missense_variant | MODERATE | Cytochrome c oxidase | CED80056 | p.Leu366Phe |
| | | | | | | | subunit 1 | | |
| Z₅ | Scaffold_28 | 1506 | C | T | missense_variant | MODERATE | ATP synthase subunit | CDZ96150 | p.Ala227Val |
| | | | | | | | 6 | | |
| Z₆ | Scaffold_43 | 464 | G | A | missense_variant | MODERATE | NADH dehydrogenase | CDZ96151 | p.Thr147Ile |
| | | | | | | | subunit 4 | | |
| Z₇ | Scaffold_43 | 753 | C | T | missense_variant | MODERATE | NADH dehydrogenase | CDZ96151 | p.Val51Ile |
| | | | | | | | subunit 4 | | |
| Z₈ | Scaffold_62 | 875 | G | A | missense_variant | MODERATE | Cytochrome c oxidase | CDZ96152 | p.Thr110Ile |
| | | | | | | | subunit 2 | | |
| Z₉ | Scaffold_102 | 2334 | G | A | missense_variant | MODERATE | NADH dehydrogenase | CDZ96153 | p.Ser181Phe |
| | | | | | | | subunits 2, 5, and | | |
| | | | | | | | related proteins | | |
| Z₁₀ | Scaffold_102 | 2590 | C | T | missense_variant | MODERATE | NADH dehydrogenase | CDZ96153 | p.Ala96Thr |
| | | | | | | | subunits 2, 5, and | | |
| | | | | | | | related proteins | | |
| Z₁₁ | Scaffold_188 | 815 | C | T | missense_variant | MODERATE | NADH dehydrogenase | CED80060 | p.Ala79Val |
| | | | | | | | subunit 6 | | |
| Z₁₂ | Scaffold_247 | 989 | C | T | missense_variant | MODERATE | Cytochrome c oxidase | CED80061 | p.Gly104Glu |
| | | | | | | | subunit 3 | | |
| Z₁₃ | Scaffold_247 | 1053 | G | A | missense_variant | MODERATE | Cytochrome c oxidase | CED80061 | p.Leu83Phe |
| | | | | | | | subunit 3 | | |
| Z₁₄ | Scaffold_52 | 1399439 | G | A | missense_variant | MODERATE | Mitochondrial | CED83134 | p.Arg244Gln |
| | | | | | | | cytochrome b2- | | |
| Z₁₅ | Scaffold_69 | 384418 | G | A | missense_variant | MODERATE | Delta 9 fatty acid | CED83656 | p.Ala149Thr |
| | | | | | | | desaturase | | |
| Z₁₆ | Scaffold_69 | 865820 | C | T | missense_variant | MODERATE | Acyl-CoA-oxidase | CED83814 | p.Leu1253Phe |
| Z₁₇ | Scaffold_79 | 346823 | G | A | missense_variant | MODERATE | Pantothenate kinase | CDZ96789 | p.Gly590Arg |
| | | | | | | | PanK and related | | |
| | | | | | | | proteins | | |
| Z₁₈ | Scaffold_79 | 1540205 | C | T | missense_variant | MODERATE | Geranylgeranyl | CDZ97186 | p.Met237Ile |
| | | | | | | | pyrophosphate | | |
| | | | | | | | synthase | | |
| Z₁₉ | Scaffold_242 | 233088 | G | A | missense_variant | MODERATE | fumarate reductase | CDZ98193 | p.Gly11Glu |
| Z₂₀ | Scaffold_262 | 420102 | G | A | missense_variant | MODERATE | Sucrose transporter | CDZ98468 | p.Thr146Ile |
| | | | | | | | and related proteins | | |
| Z₂₁ | Scaffold_262 | 562795 | C | T | missense_variant | MODERATE | Ferredoxin/ | CDZ98521 | p.Gly327Asp |
| | | | | | | | adrenodoxin reductase | | |
| Z₂₂ | Scaffold_176 | 358 | G | A | upstream_gene_variant | MODIFIER | Cytochrome b | CED80058 | |
| Z₂₃ | Scaffold_32 | 2438 | C | T | upstream_gene_variant | MODIFIER | hypothetical protein | CDZ96332 | |
| Z₂₄ | Scaffold_69 | 1342909 | G | A | upstream_gene_variant | MODIFIER | Inositol polyphosphate | CED83975 | |
| | | | | | | | multikinase, | | |
| | | | | | | | component of the | | |
| | | | | | | | ARGR transcription | | |
| | | | | | | | regulatory complex | | |
| Z₂₅ | Scaffold_262 | 1455677 | G | A | stop_gained | HIGH | G protein-coupled | CDZ98804 | p.Trp395* |
| | | | | | | | receptor, rhodopsin- | | |
| | | | | | | | like | | |

TABLE 15

| | RS | RP | Change | T | GC | CDS |
|---|---|---|---|---|---|---|
| V₁ | Scaffold_262 | 562795 | c.980G > A | missense_variant | Ferredoxin/adrenodoxin reductase | CDZ98521 |
| | Scaffold_79 | 1540205 | c.711G > A | missense_variant | Geranylgeranyl pyrophosphate | CDZ97186 |
| | | | | | synthase | |
| | Scaffold_242 | 233088 | c.32G > A | missense_variant | Succinate dehydrogenase | CDZ98193 |
| | Scaffold_32 | 2438 | c.-9C > T | upstream_gene_variant | hypothetical protein | CDZ96332 |
| | Scaffold_176 | 358 | c.-91G > A | upstream_gene_variant | cytochrome b | CED80058 |
| | Scaffold_176 | 1872 | c.*923G > A | downstream_gene_variant | cytochrome b | CED80058 |
| | Scaffold_176 | 1980 | c.*1031G > A | downstream_gene_variant | cytochrome b | CED80058 |
| | Scaffold_179 | 1525 | c.516-188G > A | intron_variant | NADH:ubiquinone/plastoquinone | CDZ96154 |
| | | | | | oxidoreductase, chain 3 | |
| | Scaffold_179 | 1780 | c.556 + 27C > T | intron_variant | NADH:ubiquinone/plastoquinone | CDZ96154 |
| | | | | | oxidoreductase, chain 3 | |
| | Scaffold_22 | 1643 | c.1096C > T | missense_variant | cytochrome c oxidase subunit 1 | CED80056 |
| | Scaffold_22 | 2524 | c.*244G > A | downstream_gene_variant | cytochrome c oxidase subunit 1 | CED80056 |
| | Scaffold_62 | 875 | c.329C > T | missense_variant | cytochrome c oxidase subunit 2 | CDZ96152 |
| | Scaffold_247 | 102 | c.*598C > T | downstream_gene_variant | cytochrome c oxidase subunit 3 | CED80061 |
| | Scaffold_247 | 989 | c.311G > A | missense_variant | cytochrome c oxidase subunit 3 | CED80061 |
| | Scaffold_28 | 236 | c.-513G > A | upstream_gene_variant | atp synthase subunit 6 | CDZ96150 |
| | Scaffold_28 | 377 | c.-372G > A | upstream_gene_variant | atp synthase subunit 6 | CDZ96150 |
| | Scaffold_28 | 1506 | c.680C > T | missense_variant | atp synthase subunit 6 | CDZ96150 |
| | Scaffold_28 | 1677 | c.*89C > T | downstream_gene_variant | atp synthase subunit 6 | CDZ96150 |
| | Scaffold_42 | 804 | c.*2C > T | downstream_gene_variant | atp synthase subunit mitochondrial | CDZ96333 |
| | Scaffold_42 | 1559 | c.*757G > A | downstream_gene_variant | atp synthase subunit mitochondrial | CDZ96333 |
| | Scaffold_43 | 464 | c.440C > T | missense_variant | nadh dehydrogenase subunit 4 | CDZ96151 |
| | Scaffold_43 | 753 | c.151G > A | missense_variant | nadh dehydrogenase subunit 4 | CDZ96151 |
| | Scaffold_43 | 1527 | c.-6246 > A | upstream_gene_variant | nadb dehydrogenase subunit 4 | CDZ96151 |
| | Scaffold_43 | 1748 | c.-845C > T | upstream_gene_variant | nadh dehydrogenase subunit 4 | CDZ96151 |
| | Scaffold_43 | 1928 | c.-1025G > A | upstream_gene_variant | nadh dehydrogenase subunit 4 | CDZ96151 |
| V₂₆ | Scaffold_247 | 1053 | c.247C > T | missense_variant | cytochrome c oxidase subunit 3 | CED80061 |

TABLE 16

| | | log2 (fold | | |
|---|---|---|---|---|
| CDS | Genomic locus | change) | q value | Uniprot ID |
| CED85080 | Scaffold_249:455827-456255 | 9.2956 | 0.00107 | Uniprot/SPTREMBL:A0A0F7SWN7 |
| CDZ98286 | Scaffold_242:525123-525779 | 6.86206 | 0.00107 | Uniprot/SPTREMBL:A0A0F7SHB1 |
| CED84930 | Scaffold_249:6618-7695 | 6.68973 | 0.00107 | Uniprot/SPTREMBL:A0A0F7SWF7 |
| CDZ96332 | Scaffold_32:2446-3134 | 6.52032 | 0.00107 | Uniprot/SPTREMBL:A0A0F7SH35 |
| CED82392 | Scaffold_33:1246311-1246833 | 6.42503 | 0.00107 | Uniprot/SPTREMBL:A0A0F7SJX8 |
| CED82668 | Scaffold_52:13001-13910 | 6.33106 | 0.00107 | Uniprot/SPTREMBL:A0A0F7SRH2 |
| CDZ97516 | Scaffold_124:123781-126151 | 6.23645 | 0.00107 | Uniprot/SPTREMBL:A0A0F7SFE4 |
| CDZ98118 | Scaffold_223:211-788 | 6.11075 | 0.03259 | Uniprot/SPTREMBL:A0A0F7SKA6 |
| CDZ97577 | Scaffold_129:458-1082 | 6.09922 | 0.0043 | Uniprot/SPTREMBL:A0A0F7SKU8 |
| CED85432 | Scaffold_249:1559276-1562689 | 6.02212 | 0.00107 | Uniprot/SPTREMBL:A0A0F7STB7 |
| CDZ96153 | Scaffold_102:1486-3157 | 6.01467 | 0.00107 | Uniprot/SPTREMBL:A0A0F7SF89 |
| CDZ96711 | Scaffold_79:125953-127385 | 5.94901 | 0.00107 | Uniprot/SPTREMBL:A0A0F7SEF7 |
| CED80057 | Scaffold_55:626-1151 | 5.84552 | 0.0043 | Uniprot/SPTREMBL:A0A0F7SIU3 |
| CDZ96413 | Scaffold_54:92765-93283 | 5.60714 | 0.00107 | Uniprot/SPTREMBL:A0A0F7SFN7 |
| CED80056 | Scaffold_22:403-2280 | 5.57479 | 0.00107 | Uniprot/SPTREMBL:A0A0F7SKT4 |
| CDZ98119 | Scaffold_232:1114-1783 | 5.55736 | 0.00107 | Uniprot/SPTREMBL:A0A0F7SKZ2 |
| CED82658 | Scaffold_33:2068732-2071114 | 5.41561 | 0.00107 | Uniprot/SPTREMBL:A0A0F7SRG4 |
| CDZ97957 | Scaffold_185:365-1651 | 5.40013 | 0.00107 | Uniprot/SPTREMBL:A0A0F7SLT0 |
| CED80059 | Scaffold_176:1042-1615 | 5.32443 | 0.00107 | Uniprot/SPTREMBL:A0A0F7SMU1 |
| CDZ96152 | Scaffold_62:372-1203 | 5.30334 | 0.00107 | Uniprot/SPTREMBL:A0A0F7SGI5 |
| CDZ96150 | Scaffold_28:748-1588 | 5.25135 | 0.00107 | Uniprot/SPTREMBL:A0A0F7SEE8 |
| CDZ97807 | Scaffold_162:611422-614937 | 5.25097 | 0.00107 | Uniprot/SPTREMBL:A0A0F7SLF3 |
| CED85517 | Scaffold_249:1853363-1853729 | 5.20577 | 0.00107 | Uniprot/SPTREMBL:A0A0F7STM0 |
| CED80058 | Scaffold_176:448-949 | 5.15189 | 0.00107 | Uniprot/SPTREMBL:A0A0F7SPC4 |
| CED85164 | Scaffold_249:714476-715290 | 5.07079 | 0.00107 | Uniprot/SPTREMBL:A0A0F7SWM1 |
| CED80061 | Scaffold_247:699-1383 | 5.01115 | 0.00107 | Uniprot/SPTREMBL:A0A0F7SKU0 |
| CED80060 | Scaffold_188:484-1047 | 4.99248 | 0.00107 | Uniprot/SPTREMBL:A0A0F7SN33 |
| CDZ96865 | Scaffold_79:565627-566760 | 4.86279 | 0.00107 | Uniprot/SPTREMBL:A0A0F7SF95 |
| CDZ96908 | Scaffold_79:687220-687896 | 4.79852 | 0.00107 | Uniprot/SPTREMBL:A0A0F7SGN7 |
| CDZ96154 | Scaffold_179:741-2193 | 4.72875 | 0.0043 | Uniprot/SPTREMBL:A0A0F7SFY0 |
| CDZ96333 | Scaffold_42:183-802 | 4.64957 | 0.00107 | Uniprot/SPTREMBL:A0A0F7SFK3 |
| CED83979 | Scaffold_69:1348663-1349659 | 4.6261 | 0.00107 | Uniprot/SPTREMBL:A0A0F7STJ2 |
| CED83252 | Scaffold_52:1717622-1718044 | 4.60829 | 0.00107 | Uniprot/SPTREMBL:A0A0F7SMG2 |
| CDZ96608 | Scaffold_54:697264-699076 | 4.57217 | 0.00107 | Uniprot/SPTREMBL:A0A0F7SFX6 |
| CED83097 | Scaffold_52:1287383-1290100 | 4.53939 | 0.00107 | Uniprot/SPTREMBL:A0A0F7SM09 |
| CED84673 | Scaffold_78:975769-976045 | 4.39949 | 0.00107 | Uniprot/SPTREMBL:A0A0F7SX11 |
| CED83815 | Scaffold_69:866921-870931 | 4.33088 | 0.00107 | Uniprot/SPTREMBL:A0A0F7STE4 |
| CED84179 | Scaffold_69:1939122-1939368 | 4.33055 | 0.00879 | Uniprot/SPTREMBL:A0A0F7SU00 |
| CDZ98103 | Scaffold_189:441479-441882 | 4.29543 | 0.00107 | Uniprot/SPTREMBL:A0A0F7SK73 |
| CED84804 | Scaffold_78:1364982-1365369 | 4.25633 | 0.00107 | Uniprot/SPTREMBL:A0A0F7SVT2 |
| CDZ96624 | Scaffold_54:747460-747837 | 4.25212 | 0.00107 | Uniprot/SPTREMBL:A0A0F7SGZ8 |
| CED82344 | Scaffold_33:1072903-1074335 | 4.23726 | 0.00107 | Uniprot/SPTREMBL:A0A0F7SNU8 |
| CDZ96883 | Scaffold_79:620425-621230 | 4.23607 | 0.00283 | Uniprot/SPTREMBL:A0A0F7SGL4 |
| CDZ97379 | Scaffold_79:2108930-2109657 | 4.19032 | 0.00107 | Uniprot/SPTREMBL:A0A0F7SJ70 |
| CDZ98774 | Scaffold_262:1369422-1370696 | 4.17583 | 0.00107 | Uniprot/SPTREMBL:A0A0F7SMU0 |
| CDZ96507 | Scaffold_54:383723-384889 | 4.13638 | 0.01841 | Uniprot/SPTREMBL:A0A0F7SHK0 |
| CDZ98452 | Scaffold_262:363391-365724 | 4.1355 | 0.00107 | Uniprot/SPTREMBL:A0A0F7SMX1 |
| CDZ96151 | Scaffold_43:7-903 | 4.12896 | 0.00107 | Uniprot/SPTREMBL:A0A0F7SE37 |
| CDZ97452 | Scaffold_79:2315499-2317423 | 4.1242 | 0.00107 | Uniprot/SPTREMBL:A0A0F7SKJ4 |
| CED84998 | Scaffold_249:216807-219954 | 4.04803 | 0.00107 | Uniprot/SPTREMBL:A0A0F7SXS0 |
| CED84637 | Scaffold_78:884903-885167 | 3.95039 | 0.00107 | Uniprot/SPTREMBL:A0A0F7SQY4 |
| CDZ97448 | Scaffold_79:2302089-2304040 | 3.94041 | 0.00107 | Uniprot/SPTREMBL:A0A0F7SIB1 |
| CED82193 | Scaffold_33:587555-588509 | 3.91561 | 0.00107 | Uniprot/SPTREMBL:A0A0F7SQ04 |
| CED82352 | Scaffold_33:1097639-1100022 | 3.88483 | 0.00107 | Uniprot/SPTREMBL:A0A0F7SJU9 |
| CDZ96740 | Scaffold_79:209909-212506 | 3.85991 | 0.00107 | Uniprot/SPTREMBL:A0A0F7SF48 |
| CED84786 | Scaffold_78:1308664-1311311 | 3.84158 | 0.00107 | Uniprot/SPTREMBL:A0A0F7STE6 |
| CDZ96633 | Scaffold_77:13254-14404 | 3.73031 | 0.00107 | Uniprot/SPTREMBL:A0A0F7SG10 |
| CED84404 | Scaffold_78:150939-152226 | 3.6789 | 0.00107 | Uniprot/SPTREMBL:A0A0F7SUN7 |
| CDZ97797 | Scaffold_162:579804-580834 | 3.66705 | 0.00283 | Uniprot/SPTREMBL:A0A0F7SLE9 |
| CDZ96601 | Scaffold_54:679325-679595 | 3.62342 | 0.00107 | Uniprot/SPTREMBL:A0A0F7SEC7 |
| CDZ96647 | Scaffold_77:41123-42029 | 3.61574 | 0.00107 | Uniprot/SPTREMBL:A0A0F7SI01 |
| CDZ98284 | Scaffold_242:519765-520056 | 3.58807 | 0.00107 | Uniprot/SPTREMBL:A0A0F7SLM1 |
| CDZ96434 | Scaffold_54:155782-156484 | 3.56523 | 0.00107 | Uniprot/SPTREMBL:A0A0F7SGI3 |
| CED84299 | Scaffold_69:2304680-2305552 | 3.54505 | 0.00107 | Uniprot/SPTREMBL:A0A0F7SUE9 |
| CDZ96748 | Scaffold_79:228516-229974 | 3.54221 | 0.00107 | Uniprot/SPTREMBL:A0A0F7SGA0 |
| CDZ97723 | Scaffold_162:378790-379092 | 3.53675 | 0.00107 | Uniprot/SPTREMBL:A0A0F7SJ97 |
| CED84714 | Scaffold_78:1088351-1090512 | 3.52201 | 0.01647 | Uniprot/SPTREMBL:A0A0F7SVC6 |
| CED85389 | Scaffold_249:1437652-1438024 | 3.48785 | 0.00635 | Uniprot/SPTREMBL:A0A0F7SXD3 |
| CDZ96731 | Scaffold_79:178450-181319 | 3.46595 | 0.00107 | Uniprot/SPTREMBL:A0A0F7SEG4 |
| CDZ97605 | Scaffold_162:65-848 | 3.46476 | 0.02837 | Uniprot/SPTREMBL:A0A0F7SGW0 |
| CDZ96885 | Scaffold_79:627282-627758 | 3.4451 | 0.00107 | Uniprot/SPTREMBL:A0A0F7SFA1 |
| CED82665 | Scaffold_52:6245-6725 | 3.42717 | 0.00107 | Uniprot/SPTREMBL:A0A0F7SQ81 |
| CED82464 | Scaffold_33:1468151-1469956 | 3.40784 | 0.00572 | Uniprot/SPTREMBL:A0A0F7SP15 |
| CED83623 | Scaffold_69:281336-282359 | 3.39893 | 0.00107 | Uniprot/SPTREMBL:A0A0F7SUF7 |
| CDZ97986 | Scaffold_189:92376-93295 | 3.39563 | 0.00763 | Uniprot/SPTREMBL:A0A0F7SGH5 |
| CED84525 | Scaffold_78:546343-546634 | 3.39311 | 0.04003 | Uniprot/SPTREMBL:A0A0F7SVG1 |
| CED83140 | Scaffold_52:1417194-1418401 | 3.36574 | 0.00107 | Uniprot/SPTREMBL:A0A0F7SRK1 |
| CDZ97423 | Scaffold_79:2235980-2237243 | 3.33272 | 0.04038 | Uniprot/SPTREMBL:A0A0F7SI94 |
| CED83508 | Scaffold_52:2532580-2533462 | 3.2865 | 0.00107 | Uniprot/SPTREMBL:A0A0F7SU56 |
| CDZ96639 | Scaffold_77:25331-25763 | 3.27732 | 0.00107 | Uniprot/SPTREMBL:A0A0F7SH10 |
| CED82131 | Scaffold_33:407214-418268 | 3.23793 | 0.002 | Uniprot/SPTREMBL:A0A0F7SL25 |
| CDZ97778 | Scaffold_162:523968-524442 | 3.23551 | 0.00107 | Uniprot/SPTREMBL:A0A0F7SJE9 |
| CDZ97625 | Scaffold_162:67635-70853 | 3.23462 | 0.00107 | Uniprot/SPTREMBL:A0A0F7SGX3 |
| CED83795 | Scaffold_69:789151-792032 | 3.19812 | 0.00107 | Uniprot/SPTREMBL:A0A0F7STA1 |
| CED83831 | Scaffold_69:919459-919771 | 3.18779 | 0.00107 | Uniprot/SPTREMBL:A0A0F7SR09 |
| CED84215 | Scaffold_69:2041512-2041896 | 3.18206 | 0.00107 | Uniprot/SPTREMBL:A0A0F7SUD7 |
| CDZ97534 | Scaffold_124:183269-184916 | 3.17103 | 0.00107 | Uniprot/SPTREMBL:A0A0F7SJL0 |
| CDZ96425 | Scaffold_54:135835-138404 | 3.1657 | 0.00572 | Uniprot/SPTREMBL:A0A0F7SEQ8 |
| CDZ96691 | Scaffold_79:68284-68653 | 3.14581 | 0.00107 | Uniprot/SPTREMBL:A0A0F7SEF0 |
| CDZ96227 | Scaffold_24:209321-215733 | 3.10759 | 0.00107 | Uniprot/SPTREMBL:A0A0F7SGW6 |
| CDZ97639 | Scaffold_162:112120-113343 | 3.09923 | 0.00107 | Uniprot/SPTREMBL:A0A0F7SJT0 |
| CED85588 | Scaffold_249:2066996-2068681 | 3.09682 | 0.002 | Uniprot/SPTREMBL:A0A0F7SZ44 |
| CED82973 | Scaffold_52:935660-938515 | 3.08913 | 0.03157 | Uniprot/SPTREMBL:A0A0F7SSD6 |
| CED85285 | Scaffold_249:1122388-1123094 | 3.0859 | 0.02777 | Uniprot/SPTREMBL:A0A0F7SXA0 |
| CDZ96723 | Scaffold_79:158597-160262 | 3.05802 | 0.00107 | Uniprot/SPTREMBL:A0A0F7SG83 |
| CED85248 | Scaffold_249:999627-1000271 | 3.04686 | 0.00818 | Uniprot/SPTREMBL:A0A0F7SYB5 |
| CED84711 | Scaffold_78:1081064-1082458 | 3.03732 | 0.00107 | Uniprot/SPTREMBL:A0A0F7ST85 |
| CED84955 | Scaffold_249:76786-77647 | 3.03601 | 0.002 | Uniprot/SPTREMBL:A0A0F7SWH0 |
| CDZ96974 | Scaffold_79:897858-899128 | 3.01336 | 0.00107 | Uniprot/SPTREMBL:A0A0F7SI66 |
| CED82598 | Scaffold_33:1903939-1904188 | 3.00633 | 0.00697 | Uniprot/SPTREMBL:A0A0F7SRB7 |
| CDZ97694 | Scaffold_162:285211-285769 | 2.98783 | 0.00107 | Uniprot/SPTREMBL:A0A0F7SK35 |
| CDZ97736 | Scaffold_162:413045-414204 | 2.98331 | 0.00107 | Uniprot/SPTREMBL:A0A0F7SFV1 |
| CDZ96625 | Scaffold_63:96-1049 | 2.97684 | 0.026 | Uniprot/SPTREMBL:A0A0F7SEZ4 |
| CDZ97068 | Scaffold_79:1181236-1182652 | 2.93609 | 0.00107 | Uniprot/SPTREMBL:A0A0F7SH49 |
| CDZ97420 | Scaffold_79:2229810-2232473 | 2.93301 | 0.0139 | Uniprot/SPTREMBL:A0A0F7SG73 |
| CED85140 | Scaffold_249:646783-648424 | 2.92845 | 0.00635 | Uniprot/SPTREMBL:A0A0F7SWV9 |
| CDZ97946 | Scaffold_162:1032808-1037153 | 2.91654 | 0.00107 | Uniprot/SPTREMBL:A0A0F7SGD0 |
| CDZ98244 | Scaffold_242:394144-394730 | 2.91373 | 0.00763 | Uniprot/SPTREMBL:A0A0F7SLI0 |
| CED83041 | Scaffold_52:1131882-1133579 | 2.91355 | 0.00107 | Uniprot/SPTREMBL:A0A0F7SNP9 |
| CED85485 | Scaffold_249:1757631-1758380 | 2.9125 | 0.03701 | Uniprot/SPTREMBL:A0A0F7SY71 |
| CDZ96603 | Scaffold_54:684615-686836 | 2.86775 | 0.00107 | Uniprot/SPTREMBL:A0A0F7SFX2 |
| CED85448 | Scaffold_249:1609292-1610258 | 2.86226 | 0.01647 | Uniprot/SPTREMBL:A0A0F7SYS0 |
| CDZ96340 | Scaffold_53:5356-7165 | 2.86174 | 0.00107 | Uniprot/SPTREMBL:A0A0F7SEM7 |
| CDZ97752 | Scaffold_162:456053-456784 | 2.8593 | 0.01003 | Uniprot/SPTREMBL:A0A0F7SLC8 |
| CDZ96938 | Scaffold_79:794992-795565 | 2.85559 | 0.00107 | Uniprot/SPTREMBL:A0A0F7SGR2 |
| CED84750 | Scaffold_78:1210728-1211551 | 2.84262 | 0.01157 | Uniprot/SPTREMBL:A0A0F7SVW4 |
| CED84654 | Scaffold_78:929313-930567 | 2.83588 | 0.0421 | Uniprot/SPTREMBL:A0A0F7SV72 |
| CED84929 | Scaffold_249:1542-2672 | 2.82389 | 0.03333 | Uniprot/SPTREMBL:A0A0F7SW16 |
| CED85266 | Scaffold_249:1061106-1062657 | 2.81591 | 0.03435 | Uniprot/SPTREMBL:A0A0F7SUQ5 |
| CED83544 | Scaffold_69:53167-54761 | 2.81175 | 0.02802 | Uniprot/SPTREMBL:A0A0F7SSD9 |
| CED83430 | Scaffold_52:2282025-2284655 | 2.81037 | 0.00107 | Uniprot/SPTREMBL:A0A0F7SSA2 |
| CDZ97896 | Scaffold_162:885528-888251 | 2.8087 | 0.00107 | Uniprot/SPTREMBL:A0A0F7SG99 |
| CDZ98193 | Scaffold_242:233056-236702 | 2.80059 | 0.00107 | Uniprot/SPTREMBL:A0A0F7SKM1 |
| CDZ96546 | Scaffold_54:514464-515814 | 2.79309 | 0.01003 | Uniprot/SPTREMBL:A0A0F7SEB6 |
| CED82513 | Scaffold_33:1637082-1638403 | 2.78715 | 0.04003 | Uniprot/SPTREMBL:A0A0F7SR22 |
| CDZ98764 | Scaffold_262:1341690-1344148 | 2.77661 | 0.00107 | Uniprot/SPTREMBL:A0A0F7SMT3 |
| CED83907 | Scaffold_69:1129431-1131705 | 2.76752 | 0.01794 | Uniprot/SPTREMBL:A0A0F7SP83 |
| CED82580 | Scaffold_33:1852517-1855025 | 2.76238 | 0.00107 | Uniprot/SPTREMBL:A0A0F7SQ15 |
| CED83518 | Scaffold_52:2553963-2555767 | 2.743 | 0.03229 | Uniprot/SPTREMBL:A0A0F7SU60 |
| CDZ97921 | Scaffold_162:964515-965232 | 2.71423 | 0.00107 | Uniprot/SPTREMBL:A0A0F7SGB4 |
| CED85536 | Scaffold_249:1908367-1911501 | 2.71336 | 0.00107 | Uniprot/SPTREMBL:A0A0F7SVG2 |
| CDZ97601 | Scaffold_155:195-516 | 2.70857 | 0.03157 | Uniprot/SPTREMBL:A0A0F7SFL1 |
| CDZ96325 | Scaffold_24:506034-507436 | 2.69145 | 0.00283 | Uniprot/SPTREMBL:A0A0F7SEM4 |
| CDZ97097 | Scaffold_79:1257573-1259549 | 2.67196 | 0.04565 | Uniprot/SPTREMBL:A0A0F7SJC9 |
| CED85390 | Scaffold_249:1438192-1439158 | 2.66706 | 0.03435 | Uniprot/SPTREMBL:A0A0F7SXN7 |
| CED85516 | Scaffold_249:1850199-1853329 | 2.65919 | 0.00107 | Uniprot/SPTREMBL:A0A0F7SVE4 |
| CED83983 | Scaffold_69:1358613-1359556 | 2.64233 | 0.00107 | Uniprot/SPTREMBL:A0A0F7SVK0 |
| CDZ98301 | Scaffold_242:578537-580259 | 2.63598 | 0.00107 | Uniprot/SPTREMBL:A0A0F7SHC5 |
| CED84180 | Scaffold_69:1939424-1940850 | 2.63185 | 0.02306 | Uniprot/SPTREMBL:A0A0F7SUC0 |
| CED85339 | Scaffold_249:1288096-1288987 | 2.63024 | 0.00879 | Uniprot/SPTREMBL:A0A0F7SXB4 |
| CED83447 | Scaffold_52:2325373-2326346 | 2.60976 | 0.00635 | Uniprot/SPTREMBL:A0A0F7SN08 |
| CDZ98703 | Scaffold_262:1147976-1153160 | 2.60584 | 0.00572 | Uniprot/SPTREMBL:A0A0F7SM10 |
| CED83390 | Scaffold_52:2154690-2155940 | 2.57361 | 0.00107 | Uniprot/SPTREMBL:A0A0F7SS86 |
| CED85564 | Scaffold_249:2007538-2009056 | 2.56166 | 0.00107 | Uniprot/SPTREMBL:A0A0F7SXT5 |
| CED85596 | Scaffold_249:2088847-2090119 | 2.55839 | 0.02962 | Uniprot/SPTREMBL:A0A0F7SVL0 |
| CDZ96960 | Scaffold_79:859494-860819 | 2.55825 | 0.02443 | Uniprot/SPTREMBL:A0A0F7SFD1 |
| CED83901 | Scaffold_69:1112099-1112693 | 2.55723 | 0.00107 | Uniprot/SPTREMBL:A0A0F7SR75 |
| CDZ96968 | Scaffold_79:880554-881657 | 2.55571 | 0.00107 | Uniprot/SPTREMBL:A0A0F7SGU9 |
| CDZ97756 | Scaffold_162:463549-466940 | 2.55281 | 0.00107 | Uniprot/SPTREMBL:A0A0F7SFY6 |
| CED84981 | Scaffold_249:153000-155119 | 2.54954 | 0.00635 | Uniprot/SPTREMBL:A0A0F7SU30 |
| CDZ96256 | Scaffold_24:312616-313991 | 2.54133 | 0.00107 | Uniprot/SPTREMBL:A0A0F7SE58 |
| CED84768 | Scaffold_78:1260836-1261396 | 2.53828 | 0.00107 | Uniprot/SPTREMBL:A0A0F7SX63 |
| CDZ97720 | Scaffold_162:372745-373132 | 2.53182 | 0.00107 | Uniprot/SPTREMBL:A0A0F7SH84 |
| CDZ96462 | Scaffold_54:239487-240962 | 2.52853 | 0.03333 | Uniprot/SPTREMBL:A0A0F7SHG2 |
| CDZ96308 | Scaffold_24:471561-474813 | 2.51609 | 0.03435 | Uniprot/SPTREMBL:A0A0F7SFJ8 |
| CDZ96398 | Scaffold_54:48224-49552 | 2.51507 | 0.00107 | Uniprot/SPTREMBL:A0A0F7SFM9 |
| CED84125 | Scaffold_69:1789791-1791079 | 2.51267 | 0.00107 | Uniprot/SPTREMBL:A0A0F7SUA0 |
| CED82597 | Scaffold_33:1901063-1903830 | 2.51046 | 0.00107 | Uniprot/SPTREMBL:A0A0F7SKE3 |
| CDZ96812 | Scaffold_79:415347-415957 | 2.50562 | 0.00358 | Uniprot/SPTREMBL:A0A0F7SIN5 |
| CED85214 | Scaffold_249:894511-897801 | 2.4834 | 0.00107 | Uniprot/SPTREMBL:A0A0F7SWT8 |
| CED83935 | Scaffold_69:1212340-1212580 | 2.44459 | 0.00879 | Uniprot/SPTREMBL:A0A0F7STN4 |
| CDZ96565 | Scaffold_54:557265-562538 | 2.44098 | 0.00107 | Uniprot/SPTREMBL:A0A0F7SEY2 |
| CDZ96800 | Scaffold_79:377379-378952 | 2.43835 | 0.03632 | Uniprot/SPTREMBL:A0A0F7SF69 |
| CDZ97029 | Scaffold_79:1059984-1060272 | 2.43064 | 0.02802 | Uniprot/SPTREMBL:A0A0F7SI95 |
| CDZ97443 | Scaffold_79:2290162-2292224 | 2.41806 | 0.02473 | Uniprot/SPTREMBL:A0A0F7SIA8 |
| CED83766 | Scaffold_69:691513-694257 | 2.41186 | 0.00358 | Uniprot/SPTREMBL:A0A0F7SQQ1 |
| CDZ97945 | Scaffold_162:1031822-1032537 | 2.40755 | 0.02802 | Uniprot/SPTREMBL:A0A0F7SHT1 |
| CDZ96911 | Scaffold_79:690843-693362 | 2.38478 | 0.00107 | Uniprot/SPTREMBL:A0A0F7SEN6 |
| CDZ98503 | Scaffold_262:516975-517254 | 2.38051 | 0.00358 | Uniprot/SPTREMBL:A0A0F7SLD8 |
| CED84099 | Scaffold_69:1712578-1712954 | 2.37587 | 0.00107 | Uniprot/SPTREMBL:A0A0F7STV4 |
| CDZ97312 | Scaffold_79:1919826-1920493 | 2.37313 | 0.00107 | Uniprot/SPTREMBL:A0A0F7SK29 |
| CED82663 | Scaffold_33:2085699-2086479 | 2.36106 | 0.00107 | Uniprot/SPTREMBL:A0A0F7SRG7 |
| CED84677 | Scaffold_78:988591-991029 | 2.35953 | 0.00107 | Uniprot/SPTREMBL:A0A0F7SR25 |
| CED83843 | Scaffold_69:952349-955303 | 2.35584 | 0.00818 | Uniprot/SPTREMBL:A0A0F7SV82 |
| CED82395 | Scaffold_33:1255820-1258390 | 2.35553 | 0.00107 | Uniprot/SPTREMBL:A0A0F7SPH8 |
| CED85400 | Scaffold_249:1464663-1466595 | 2.35205 | 0.00107 | Uniprot/SPTREMBL:A0A0F7SXS5 |
| CED82482 | Scaffold_33:1524955-1525327 | 2.33456 | 0.00107 | Uniprot/SPTREMBL:A0A0F7SK45 |
| CED82114 | Scaffold_33:344547-346439 | 2.33295 | 0.00107 | Uniprot/SPTREMBL:A0A0F7SN68 |
| CDZ96998 | Scaffold_79:961487-962221 | 2.33028 | 0.00283 | Uniprot/SPTREMBL:A0A0F7SH05 |
| CDZ96634 | Scaffold_77:15676-16842 | 2.32402 | 0.00107 | Uniprot/SPTREMBL:A0A0F7SH06 |
| CDZ98558 | Scaffold_262:709808-712125 | 2.31029 | 0.00107 | Uniprot/SPTREMBL:A0A0F7SLI7 |
| CED85083 | Scaffold_249:460456-461478 | 2.30642 | 0.02919 | Uniprot/SPTREMBL:A0A0F7SXY0 |
| CDZ96978 | Scaffold_79:911819-914066 | 2.2979 | 0.03435 | Uniprot/SPTREMBL:A0A0F7SGV8 |
| CDZ97884 | Scaffold_162:847559-849948 | 2.28895 | 0.02473 | Uniprot/SPTREMBL:A0A0F7SKD6 |
| CED83769 | Scaffold_69:699438-701924 | 2.28794 | 0.00107 | Uniprot/SPTREMBL:A0A0F7SSZ9 |
| CED85326 | Scaffold_249:1257258-1258493 | 2.2837 | 0.00107 | Uniprot/SPTREMBL:A0A0F7SUY7 |
| CDZ96782 | Scaffold_79:323429-325820 | 2.27037 | 0.00107 | Uniprot/SPTREMBL:A0A0F7SIK6 |
| CDZ97481 | Scaffold_124:7774-13138 | 2.25897 | 0.0043 | Uniprot/SPTREMBL:A0A0F7SFC7 |
| CED85048 | Scaffold_249:370221-371540 | 2.25614 | 0.00697 | Uniprot/SPTREMBL:A0A0F7SXU8 |
| CED85332 | Scaffold_249:1271082-1271657 | 2.24969 | 0.02351 | Uniprot/SPTREMBL:A0A0F7ST43 |
| CED82842 | Scaffold_52:560142-562537 | 2.24235 | 0.04884 | Uniprot/SPTREMBL:A0A0F7SL74 |
| CDZ96732 | Scaffold_79:181587-183236 | 2.23715 | 0.00107 | Uniprot/SPTREMBL:A0A0F7SIG1 |
| CED83940 | Scaffold_69:1230023-1233189 | 2.23141 | 0.00572 | Uniprot/SPTREMBL:Q3HR17 |
| CDZ98646 | Scaffold_262:962599-964005 | 2.22002 | 0.00107 | Uniprot/SPTREMBL:A0A0F7SI49 |
| CED84596 | Scaffold_78:751045-752053 | 2.21506 | 0.00107 | Uniprot/SPTREMBL:A0A0F7ST01 |
| CED84309 | Scaffold_69:2328564-2329971 | 2.18139 | 0.00818 | Uniprot/SPTREMBL:A0A0F7SUF6 |
| CED85421 | Scaffold_249:1522642-1523545 | 2.1799 | 0.00107 | Uniprot/SPTREMBL:A0A0F7SV61 |
| CDZ96632 | Scaffold_77:12435-12783 | 2.17855 | 0.02637 | Uniprot/SPTREMBL:A0A0F7SHY4 |
| CED82055 | Scaffold_33:159801-160443 | 2.16592 | 0.00107 | Uniprot/SPTREMBL:A0A0F7SN80 |
| CED84932 | Scaffold_249:11741-12335 | 2.15937 | 0.00107 | Uniprot/SPTREMBL:A0A0F7SRU4 |
| CED83176 | Scaffold_52:1503852-1505330 | 2.15895 | 0.00107 | Uniprot/SPTREMBL:A0A0F7SP03 |
| CED85184 | Scaffold_249:785518-789209 | 2.15362 | 0.00107 | Uniprot/SPTREMBL:A0A0F7SWN9 |
| CED83158 | Scaffold_52:1456975-1458575 | 2.15067 | 0.01101 | Uniprot/SPTREMBL:A0A0F7SSX2 |
| CED84360 | Scaffold_78:4618-6613 | 2.14217 | 0.00107 | Uniprot/SPTREMBL:A0A0F7SV22 |
| CDZ96606 | Scaffold_54:691857-692139 | 2.13837 | 0.03188 | Uniprot/SPTREMBL:A0A0F7SEC8 |
| CDZ98281 | Scaffold_242:511970-514140 | 2.12939 | 0.03362 | Uniprot/SPTREMBL:A0A0F7SHA8 |
| CED83450 | Scaffold_52:2332008-2335986 | 2.12535 | 0.00107 | Uniprot/SPTREMBL:A0A0F7SSD2 |
| CDZ96255 | Scaffold_24:304621-312364 | 2.12075 | 0.00107 | Uniprot/SPTREMBL:A0A0F7SEK2 |
| CED84965 | Scaffold_249:100476-100977 | 2.12015 | 0.02687 | Uniprot/SPTREMBL:A0A0F7SWH8 |
| CED83977 | Scaffold_69:1344500-1346015 | 2.11941 | 0.03518 | Uniprot/SPTREMBL:A0A0F7SPB1 |
| CDZ96598 | Scaffold_54:674693-675994 | 2.11801 | 0.002 | Uniprot/SPTREMBL:A0A0F7SFW9 |
| CED85198 | Scaffold_249:838732-840721 | 2.11044 | 0.0431 | Uniprot/SPTREMBL:A0A0F7SY45 |
| CDZ98037 | Scaffold_189:240085-243335 | 2.1099 | 0.0367 | Uniprot/SPTREMBL:A0A0F7SM63 |
| CED84166 | Scaffold_69:1902914-1907183 | 2.10358 | 0.00107 | Uniprot/SPTREMBL:A0A0F7SRS9 |
| CDZ96253 | Scaffold_24:294036-295659 | 2.09696 | 0.00107 | Uniprot/SPTREMBL:A0A0F7SFH1 |
| CDZ96587 | Scaffold_54:635972-638782 | 2.07411 | 0.00283 | Uniprot/SPTREMBL:A0A0F7SHU1 |
| CDZ97406 | Scaffold_79:2190879-2192026 | 2.06531 | 0.01217 | Uniprot/SPTREMBL:A0A0F7SF75 |
| CED82118 | Scaffold_33:355172-355794 | 2.06453 | 0.00107 | Uniprot/SPTREMBL:A0A0F7SPR7 |
| CED84712 | Scaffold_78:1082888-1085081 | 2.04606 | 0.00107 | Uniprot/SPTREMBL:A0A0F7SR59 |
| CED83235 | Scaffold_52:1658608-1660253 | 2.03709 | 0.02837 | Uniprot/SPTREMBL:A0A0F7SRX7 |
| CDZ96808 | Scaffold_79:402194-403759 | 2.01274 | 0.00107 | Uniprot/SPTREMBL:A0A0F7SGF8 |
| CED84189 | Scaffold_69:1965278-1968656 | 2.00903 | 0.00107 | Uniprot/SPTREMBL:A0A0F7SU09 |
| CDZ96972 | Scaffold_79:892224-894290 | 2.00813 | 0.00107 | Uniprot/SPTREMBL:A0A0F7SJ16 |
| CDZ98157 | Scaffold_242:119690-120995 | 2.00799 | 0.01157 | Uniprot/SPTREMBL:A0A0F7SMG5 |
| CED85202 | Scaffold_249:856230-864330 | 2.00782 | 0.00107 | Uniprot/SPTREMBL:A0A0F7SSN3 |
| CDZ97710 | Scaffold_162:350139-350415 | 2.00424 | 0.00697 | Uniprot/SPTREMBL:A0A0F7SH77 |
| CED82504 | Scaffold_33:1607291-1608419 | 2.0026 | 0.00107 | Uniprot/SPTREMBL:A0A0F7SP52 |
| CDZ98740 | Scaffold_262:1258470-1261686 | 2.00231 | 0.00107 | Uniprot/SPTREMBL:A0A0F7SK26 |
| CDZ98359 | Scaffold_262:82976-84729 | 1.99765 | 0.01794 | Uniprot/SPTREMBL:A0A0F7SLU2 |
| CED84345 | Scaffold_69:2445860-2450055 | 1.99755 | 0.00107 | Uniprot/SPTREMBL:A0A0F7SV09 |
| CDZ96501 | Scaffold_54:372224-373353 | 1.99602 | 0.00635 | Uniprot/SPTREMBL:A0A0F7SEA7 |
| CED84559 | Scaffold_78:652640-653728 | 1.99303 | 0.00107 | Uniprot/SPTREMBL:A0A0F7SUY8 |
| CED82642 | Scaffold_33:2021875-2023953 | 1.98413 | 0.00107 | Uniprot/SPTREMBL:A0A0F7SKH3 |
| CED82491 | Scaffold_33:1571741-1572704 | 1.96965 | 0.00107 | Uniprot/SPTREMBL:A0A0F7SM44 |
| CDZ98294 | Scaffold_242:549453-552382 | 1.96744 | 0.01332 | Uniprot/SPTREMBL:A0A0F7SLM8 |
| CDZ96892 | Scaffold_79:643491-645980 | 1.96608 | 0.00107 | Uniprot/SPTREMBL:A0A0F7SIU6 |
| CED82781 | Scaffold_52:377187-379075 | 1.96467 | 0.03632 | Uniprot/SPTREMBL:A0A0F7SMZ9 |
| CED84469 | Scaffold_78:379871-381445 | 1.96299 | 0.0161 | Uniprot/SPTREMBL:A0A0F7SUT6 |
| CED83869 | Scaffold_69:1022462-1022876 | 1.9615 | 0.02177 | Uniprot/SPTREMBL:A0A0F7STC4 |
| CDZ98639 | Scaffold_262:942397-945182 | 1.96071 | 0.00697 | Uniprot/SPTREMBL:A0A0F7SMC3 |
| CED82294 | Scaffold_33:915824-923046 | 1.95891 | 0.00107 | Uniprot/SPTREMBL:A0A0F7SNM9 |
| CDZ98287 | Scaffold_242:526295-527209 | 1.95446 | 0.01694 | Uniprot/SPTREMBL:A0A0F7SMN1 |
| CDZ98041 | Scaffold_189:257441-258978 | 1.95439 | 0.00358 | Uniprot/SPTREMBL:A0A0F7SGM4 |
| CDZ96788 | Scaffold_79:341612-343354 | 1.95437 | 0.03157 | Uniprot/SPTREMBL:A0A0F7SGC5 |
| CDZ97716 | Scaffold_162:364590-367041 | 1.95203 | 0.00107 | Uniprot/SPTREMBL:A0A0F7SFU3 |
| CED83610 | Scaffold_69:242322-244080 | 1.92797 | 0.00107 | Uniprot/SPTREMBL:A0A0F7SST7 |
| CDZ96286 | Scaffold_24:397274-398963 | 1.92503 | 0.00107 | Uniprot/SPTREMBL:A0A0F7SE64 |
| CED84538 | Scaffold_78:578277-581045 | 1.90782 | 0.03932 | Uniprot/SPTREMBL:A0A0F7SWS5 |
| CED84356 | Scaffold_69:2490030-2490654 | 1.90252 | 0.00107 | Uniprot/SPTREMBL:A0A0F7SS99 |
| CED85504 | Scaffold_249:1810396-1812070 | 1.90035 | 0.00358 | Uniprot/SPTREMBL:A0A0F7SXP5 |
| CED82460 | Scaffold_33:1453523-1455238 | 1.89525 | 0.00107 | Uniprot/SPTREMBL:A0A0F7SPP5 |
| CED82775 | Scaffold_52:362164-364734 | 1.89452 | 0.00283 | Uniprot/SPTREMBL:A0A0F7SQD0 |
| CED83321 | Scaffold_52:1931031-1933041 | 1.88777 | 0.002 | Uniprot/SPTREMBL:A0A0F7SPI4 |
| CDZ96906 | Scaffold_79:681395-683346 | 1.88628 | 0.01694 | Uniprot/SPTREMBL:A0A0F7SEN4 |
| CED84096 | Scaffold_69:1707265-1708032 | 1.88125 | 0.0161 | Uniprot/SPTREMBL:A0A0F7SRN0 |
| CED85628 | Scaffold_249:2197535-2201856 | 1.8811 | 0.00107 | Uniprot/SPTREMBL:A0A0F7SZ68 |
| CED82156 | Scaffold_33:478691-480487 | 1.88089 | 0.0043 | Uniprot/SPTREMBL:A0A0F7SL33 |
| CDZ96504 | Scaffold_54:378291-378717 | 1.88061 | 0.00283 | Uniprot/SPTREMBL:A0A0F7SGP6 |
| CED85328 | Scaffold_249:1262299-1263857 | 1.87508 | 0.0043 | Uniprot/SPTREMBL:A0A0F7SYK9 |
| CED83818 | Scaffold_69:877698-879991 | 1.86052 | 0.00107 | Uniprot/SPTREMBL:A0A0F7SV21 |
| CDZ97194 | Scaffold_79:1560834-1562423 | 1.84772 | 0.0043 | Uniprot/SPTREMBL:A0A0F7SIN2 |
| CDZ98812 | Scaffold_262:1476547-1477648 | 1.84706 | 0.00697 | Uniprot/SPTREMBL:A0A0F7SP59 |
| CDZ97787 | Scaffold_162:549563-550935 | 1.83915 | 0.00107 | Uniprot/SPTREMBL:A0A0F7SLE1 |
| CDZ98454 | Scaffold_262:367663-368762 | 1.83557 | 0.04815 | Uniprot/SPTREMBL:A0A0F7SLZ5 |
| CED83570 | Scaffold_69:136793-137232 | 1.83385 | 0.00763 | Uniprot/SPTREMBL:A0A0F7SSP1 |
| CDZ98779 | Scaffold_262:1387128-1388066 | 1.83114 | 0.02143 | Uniprot/SPTREMBL:A0A0F7SMW1 |
| CED82892 | Scaffold_52:712686-713696 | 1.82093 | 0.002 | Uniprot/SPTREMBL:A0A0F7SLA9 |
| CDZ98620 | Scaffold_262:884277-885713 | 1.82088 | 0.00107 | Uniprot/SPTREMBL:A0A0F7SJJ9 |
| CED84456 | Scaffold_78:343882-347639 | 1.80468 | 0.00107 | Uniprot/SPTREMBL:A0A0F7SSL8 |
| CED85327 | Scaffold_249:1258917-1262061 | 1.79428 | 0.00107 | Uniprot/SPTREMBL:A0A0F7ST41 |
| CED83941 | Scaffold_69:1233633-1235992 | 1.7932 | 0.00107 | Uniprot/SPTREMBL:A0A0F7SRB6 |
| CED82524 | Scaffold_33:1668538-1669641 | 1.79313 | 0.04455 | Uniprot/SPTREMBL:A0A0F7SP69 |
| CED85282 | Scaffold_249:1112034-1112403 | 1.79149 | 0.00107 | Uniprot/SPTREMBL:A0A0F7SSW3 |
| CED84172 | Scaffold_69:1923729-1924227 | 1.7889 | 0.01101 | Uniprot/SPTREMBL:A0A0F7SPM4 |
| CDZ96439 | Scaffold_54:171347-173858 | 1.78847 | 0.00107 | Uniprot/SPTREMBL:A0A0F7SGI8 |
| CDZ96760 | Scaffold_79:261484-264763 | 1.78807 | 0.0043 | Uniprot/SPTREMBL:A0A0F7SF56 |
| CDZ97680 | Scaffold_162:241932-249389 | 1.78266 | 0.00107 | Uniprot/SPTREMBL:A0A0F7SH15 |
| CED83749 | Scaffold_69:647207-648008 | 1.7682 | 0.00763 | Uniprot/SPTREMBL:A0A0F7SSY3 |
| CED84996 | Scaffold_249:211362-212285 | 1.76735 | 0.01647 | Uniprot/SPTREMBL:A0A0F7SU50 |
| CDZ97494 | Scaffold_124:53255-54125 | 1.76701 | 0.01332 | Uniprot/SPTREMBL:A0A0F7SJH9 |
| CDZ98520 | Scaffold_262:561685-561949 | 1.76251 | 0.03739 | Uniprot/SPTREMBL:A0A0F7SJA9 |
| CED82328 | Scaffold_33:1021691-1022108 | 1.76246 | 0.00107 | Uniprot/SPTREMBL:A0A0F7SQL5 |
| CDZ97939 | Scaffold_162:1013551-1014587 | 1.75145 | 0.002 | Uniprot/SPTREMBL:A0A0F7SKJ1 |
| CDZ96930 | Scaffold_79:746360-747555 | 1.75114 | 0.00572 | Uniprot/SPTREMBL:A0A0F7SFC1 |
| CED82946 | Scaffold_52:860371-866416 | 1.74633 | 0.00283 | Uniprot/SPTREMBL:A0A0F7SNI9 |
| CED83711 | Scaffold_69:554013-556123 | 1.74598 | 0.00107 | Uniprot/SPTREMBL:A0A0F7SQJ8 |
| CED82123 | Scaffold_33:388987-389410 | 1.74366 | 0.01053 | Uniprot/SPTREMBL:A0A0F7SPS1 |
| CED82360 | Scaffold_33:1130243-1130913 | 1.73312 | 0.00107 | Uniprot/SPTREMBL:A0A0F7SPF3 |
| CDZ97779 | Scaffold_162:525829-527237 | 1.733 | 0.01694 | Uniprot/SPTREMBL:A0A0F7SK88 |
| CED85569 | Scaffold_249:2019949-2021723 | 1.72317 | 0.01945 | Uniprot/SPTREMBL:A0A0F7SXT8 |
| CED83436 | Scaffold_52:2300030-2305206 | 1.71845 | 0.0431 | Uniprot/SPTREMBL:A0A0F7SPX5 |
| CDZ98665 | Scaffold_262:1028338-1030601 | 1.71812 | 0.00358 | Uniprot/SPTREMBL:A0A0F7SJN5 |
| CED85020 | Scaffold_249:296664-298545 | 1.71623 | 0.01157 | Uniprot/SPTREMBL:A0A0F7SWL0 |
| CED84604 | Scaffold_78:779180-781208 | 1.71439 | 0.00107 | Uniprot/SPTREMBL:A0A0F7SV30 |
| CED82780 | Scaffold_52:376708-377149 | 1.70936 | 0.02871 | Uniprot/SPTREMBL:A0A0F7SQD4 |
| CDZ97281 | Scaffold_79:1836628-1840608 | 1.69661 | 0.00107 | Uniprot/SPTREMBL:A0A0F7SF24 |
| CED83439 | Scaffold_52:2309892-2311510 | 1.69193 | 0.02637 | Uniprot/SPTREMBL:A0A0F7SS19 |
| CED82079 | Scaffold_33:239200-241650 | 1.69016 | 0.002 | Uniprot/SPTREMBL:A0A0F7SN39 |
| CDZ98224 | Scaffold_242:327901-329763 | 1.68977 | 0.04003 | Uniprot/SPTREMBL:A0A0F7SLH2 |
| CED82722 | Scaffold_52:182117-184588 | 1.68822 | 0.00107 | Uniprot/SPTREMBL:A0A0F7SKP4 |
| CDZ97375 | Scaffold_79:2093793-2097753 | 1.68491 | 0.02103 | Uniprot/SPTREMBL:A0A0F7SG41 |
| CDZ97123 | Scaffold_79:1339460-1339748 | 1.67557 | 0.00879 | Uniprot/SPTREMBL:A0A0F7SH81 |
| CED84572 | Scaffold_78:691147-691673 | 1.6727 | 0.03932 | Uniprot/SPTREMBL:A0A0F7SQS5 |
| CDZ97512 | Scaffold_124:111983-114421 | 1.65771 | 0.00283 | Uniprot/SPTREMBL:A0A0F7SKN5 |
| CED85019 | Scaffold_249:294296-296444 | 1.65616 | 0.00283 | Uniprot/SPTREMBL:A0A0F7SW64 |
| CED82324 | Scaffold_33:1008644-1009307 | 1.65351 | 0.04494 | Uniprot/SPTREMBL:A0A0F7SNU0 |
| CED82508 | Scaffold_33:1616764-1620298 | 1.65261 | 0.02995 | Uniprot/SPTREMBL:A0A0F7SR17 |
| CDZ97693 | Scaffold_162:282885-284685 | 1.6427 | 0.04565 | Uniprot/SPTREMBL:A0A0F7SJ63 |
| CED82289 | Scaffold_33:901370-903029 | 1.64196 | 0.02177 | Uniprot/SPTREMBL:A0A0F7SNM3 |
| CED83145 | Scaffold_52:1426474-1427363 | 1.63568 | 0.00501 | Uniprot/SPTREMBL:A0A0F7SRK6 |
| CED85454 | Scaffold_249:1633248-1635799 | 1.63511 | 0.01053 | Uniprot/SPTREMBL:A0A0F7SXI7 |
| CED82385 | Scaffold_33:1213652-1214862 | 1.63026 | 0.00107 | Uniprot/SPTREMBL:A0A0F7SPH1 |
| CED85322 | Scaffold_249:1240882-1244102 | 1.62685 | 0.00107 | Uniprot/SPTREMBL:A0A0F7ST39 |
| CED84535 | Scaffold_78:571126-573958 | 1.62373 | 0.00107 | Uniprot/SPTREMBL:A0A0F7SVH0 |
| CED84214 | Scaffold_69:2039858-2041252 | 1.61855 | 0.00283 | Uniprot/SPTREMBL:A0A0F7SU34 |
| CDZ98683 | Scaffold_262:1084236-1085358 | 1.61606 | 0.03299 | Uniprot/SPTREMBL:A0A0F7SLR4 |
| CDZ96646 | Scaffold_77:37852-39974 | 1.61544 | 0.0412 | Uniprot/SPTREMBL:A0A0F7SED6 |
| CDZ97655 | Scaffold_162:169391-171313 | 1.61044 | 0.00107 | Uniprot/SPTREMBL:A0A0F7SGZ1 |
| CED82435 | Scaffold_33:1375657-1376321 | 1.60294 | 0.03362 | Uniprot/SPTREMBL:A0A0F7SPL1 |
| CDZ96353 | Scaffold_53:39630-41165 | 1.60113 | 0.01332 | Uniprot/SPTREMBL:A0A0F7SFK7 |
| CED85316 | Scaffold_249:1219985-1222284 | 1.5994 | 0.00107 | Uniprot/SPTREMBL:A0A0F7SUY1 |
| CED85551 | Scaffold_249:1966372-1966880 | 1.5983 | 0.02306 | Uniprot/SPTREMBL:A0A0F7SVH5 |
| CED82905 | Scaffold_52:739430-741872 | 1.5972 | 0.00283 | Uniprot/SPTREMBL:A0A0F7SQX4 |
| CED82119 | Scaffold_33:356446-357504 | 1.59584 | 0.02777 | Uniprot/SPTREMBL:A0A0F7SN74 |
| CDZ97090 | Scaffold_79:1242177-1243818 | 1.58623 | 0.00358 | Uniprot/SPTREMBL:A0A0F7SFM3 |
| CDZ98290 | Scaffold_242:536964-539211 | 1.58604 | 0.00107 | Uniprot/SPTREMBL:A0A0F7SIT5 |
| CDZ97832 | Scaffold_162:704190-706731 | 1.58336 | 0.02802 | Uniprot/SPTREMBL:A0A0F7SLG7 |
| CED85078 | Scaffold_249:452193-453817 | 1.58042 | 0.00572 | Uniprot/SPTREMBL:A0A0F7SXX7 |
| CDZ97342 | Scaffold_79:2008848-2011495 | 1.57743 | 0.00358 | Uniprot/SPTREMBL:A0A0F7SKA1 |
| CDZ98442 | Scaffold_262:332477-334363 | 1.57599 | 0.00107 | Uniprot/SPTREMBL:A0A0F7SMW0 |
| CDZ98586 | Scaffold_262:790738-792475 | 1.57232 | 0.036 | Uniprot/SPTREMBL:A0A0F7SHZ1 |
| CDZ96795 | Scaffold_79:367786-368691 | 1.57118 | 0.00107 | Uniprot/SPTREMBL:A0A0F7SF68 |
| CED84312 | Scaffold_69:2335968-2339524 | 1.56304 | 0.00107 | Uniprot/SPTREMBL:A0A0F7SQ38 |
| CED85566 | Scaffold_249:2010817-2014453 | 1.56205 | 0.00818 | Uniprot/SPTREMBL:A0A0F7SVJ0 |
| CDZ96533 | Scaffold_54:469308-470639 | 1.54987 | 0.00107 | Uniprot/SPTREMBL:A0A0F7SFT7 |
| CED83799 | Scaffold_69:806596-808829 | 1.54674 | 0.01555 | Uniprot/SPTREMBL:A0A0F7ST65 |
| CDZ97301 | Scaffold_79:1895673-1899713 | 1.54005 | 0.00107 | Uniprot/SPTREMBL:A0A0F7SF28 |
| CDZ98863 | Scaffold_262:1643499-1645000 | 1.53807 | 0.015 | Uniprot/SPTREMBL:A0A0F7SMR4 |
| CDZ97626 | Scaffold_162:72630-75282 | 1.53645 | 0.02871 | Uniprot/SPTREMBL:A0A0F7SFM5 |
| CED85634 | Scaffold_249:2227699-2228797 | 1.53581 | 0.00107 | Uniprot/SPTREMBL:A0A0F7SXX2 |
| CED85324 | Scaffold_249:1248991-1251887 | 1.53192 | 0.02802 | Uniprot/SPTREMBL:A0A0F7SXA4 |
| CED82417 | Scaffold_33:1319300-1322122 | 1.53133 | 0.04244 | Uniprot/SPTREMBL:A0A0F7SJZ6 |
| CDZ97678 | Scaffold_162:232784-241715 | 1.52463 | 0.0043 | Uniprot/SPTREMBL:A0A0F7SIX8 |
| CDZ96274 | Scaffold_24:357108-358373 | 1.51225 | 0.005005 | Uniprot/SPTREMBL:A0A0F7SG98 |
| CDZ98521 | Scaffold_262:562010-563865 | 1.50923 | 0.043893 | Uniprot/SPTREMBL:A0A0F7SHS8 |
| CDZ98400 | Scaffold_262:210340-212288 | 1.50678 | 0.021773 | Uniprot/SPTREMBL:A0A0F7SJ22 |
| CED83916 | Scaffold_69:1154024-1156799 | 1.50497 | 0.001074 | Uniprot/SPTREMBL:A0A0F7SR83 |
| CDZ96728 | Scaffold_79:169536-171538 | 1.50386 | 0.001074 | Uniprot/SPTREMBL:A0A0F7SG87 |
| CED84058 | Scaffold_69:1585666-1587120 | 1.50001 | 0.04038 | Uniprot/SPTREMBL:A0A0F7SVP8 |
| CDZ97260 | Scaffold_79:1781498-1784170 | 1.49942 | 0.001074 | Uniprot/SPTREMBL:A0A0F7SFY9 |
| CDZ98006 | Scaffold_189:153436-154789 | 1.49578 | 0.011005 | Uniprot/SPTREMBL:A0A0F7SGJ4 |
| CDZ97704 | Scaffold_162:327605-328202 | 1.49284 | 0.023995 | Uniprot/SPTREMBL:A0A0F7SK48 |
| CED85192 | Scaffold_249:819695-822477 | 1.47714 | 0.001074 | Uniprot/SPTREMBL:A0A0F7SSM3 |
| CED82260 | Scaffold_33:802880-804905 | 1.47606 | 0.001074 | Uniprot/SPTREMBL:A0A0F7SNS6 |
| CDZ96526 | Scaffold_54:453538-454510 | 1.47498 | 0.001074 | Uniprot/SPTREMBL:A0A0F7SEB2 |
| CED85479 | Scaffold_249:1739097-1739458 | 1.46899 | 0.036699 | Uniprot/SPTREMBL:A0A0F7SXK3 |
| CDZ97261 | Scaffold_79:1784392-1786248 | 1.46845 | 0.031209 | Uniprot/SPTREMBL:A0A0F7SF20 |
| CDZ98595 | Scaffold_262:816862-820045 | 1.46285 | 0.002826 | Uniprot/SPTREMBL:A0A0F7SJH7 |
| CDZ96765 | Scaffold_79:275148-276949 | 1.46086 | 0.005005 | Uniprot/SPTREMBL:A0A0F7SF58 |
| CED82370 | Scaffold_33:1159209-1160902 | 1.46051 | 0.003585 | Uniprot/SPTREMBL:A0A0F7SPF9 |
| CED84441 | Scaffold_78:281458-282484 | 1.45883 | 0.039325 | Uniprot/SPTREMBL:A0A0F7SSK4 |
| CED84821 | Scaffold_78:1400404-1402626 | 1.45723 | 0.001998 | Uniprot/SPTREMBL:A0A0F7STL5 |
| CED83992 | Scaffold_69:1384528-1390584 | 1.45703 | 0.003585 | Uniprot/SPTREMBL:A0A0F7SPB8 |
| CED85625 | Scaffold_249:2187157-2189464 | 1.45685 | 0.001074 | Uniprot/SPTREMBL:A0A0F7SYG0 |
| CED82584 | Scaffold_33:1862368-1865218 | 1.45497 | 0.004297 | Uniprot/SPTREMBL:A0A0F7SPF4 |
| CDZ98305 | Scaffold_242:592604-596344 | 1.45288 | 0.002826 | Uniprot/SPTREMBL:A0A0F7SIU5 |
| CDZ96872 | Scaffold_79:584983-586708 | 1.45215 | 0.001074 | Uniprot/SPTREMBL:A0A0F7SIT0 |
| CED85203 | Scaffold_249:864947-866126 | 1.44951 | 0.001074 | Uniprot/SPTREMBL:A0A0F7SY49 |
| CDZ96187 | Scaffold_24:77879-79734 | 1.44778 | 0.005005 | Uniprot/SPTREMBL:A0A0F7SGN5 |
| CED82635 | Scaffold_33:2007011-2008851 | 1.44289 | 0.039087 | Uniprot/SPTREMBL:A0A0F7SQ56 |
| CDZ97519 | Scaffold_124:131824-135131 | 1.43614 | 0.008176 | Uniprot/SPTREMBL:A0A0F7SJJ8 |
| CDZ98632 | Scaffold_262:919319-922346 | 1.43541 | 0.00697 | Uniprot/SPTREMBL:A0A0F7SNG6 |
| CED82723 | Scaffold_52:185738-187226 | 1.43248 | 0.039087 | Uniprot/SPTREMBL:A0A0F7SRM0 |
| CED83667 | Scaffold_69:419784-423977 | 1.4323 | 0.033335 | Uniprot/SPTREMBL:A0A0F7SNP5 |
| CED84931 | Scaffold_249:9545-10343 | 1.43044 | 0.001998 | Uniprot/SPTREMBL:A0A0F7STW7 |
| CDZ97346 | Scaffold_79:2017105-2018316 | 1.42427 | 0.001998 | Uniprot/SPTREMBL:A0A0F7SF46 |
| CED82879 | Scaffold_52:682296-684509 | 1.42222 | 0.023063 | Uniprot/SPTREMBL:A0A0F7SQF9 |
| CED85363 | Scaffold_249:1364200-1366036 | 1.41596 | 0.028707 | Uniprot/SPTREMBL:A0A0F7SYN0 |
| CED83093 | Scaffold_52:1272752-1277459 | 1.40238 | 0.012168 | Uniprot/SPTREMBL:A0A0F7SSQ8 |
| CDZ97515 | Scaffold_124:121733-122976 | 1.40111 | 0.008176 | Uniprot/SPTREMBL:A0A0F7SGL2 |
| CED85082 | Scaffold_249:459159-460208 | 1.39994 | 0.001998 | Uniprot/SPTREMBL:A0A0F7SS76 |
| CED82416 | Scaffold_33:1318149-1318509 | 1.39146 | 0.006352 | Uniprot/SPTREMBL:A0A0F7SLW8 |
| CDZ96555 | Scaffold_54:529030-531464 | 1.39109 | 0.001998 | Uniprot/SPTREMBL:A0A0F7SEX9 |
| CED83550 | Scaffold_69:64295-67968 | 1.38618 | 0.030842 | Uniprot/SPTREMBL:A0A0F7SSM5 |
| CED84396 | Scaffold_78:126951-127725 | 1.38546 | 0.038728 | Uniprot/SPTREMBL:A0A0F7SSE3 |
| CED85377 | Scaffold_249:1408739-1411342 | 1.38479 | 0.00946 | Uniprot/SPTREMBL:A0A0F7ST75 |
| CED83404 | Scaffold_52:2200688-2204341 | 1.38262 | 0.006352 | Uniprot/SPTREMBL:A0A0F7SRT1 |
| CDZ96537 | Scaffold_54:476312-485671 | 1.37497 | 0.002826 | Uniprot/SPTREMBL:A0A0F7SHN7 |
| CED84907 | Scaffold_78:1683401-1684137 | 1.37476 | 0.030421 | Uniprot/SPTREMBL:A0A0F7SRT5 |
| CDZ98870 | Scaffold_262:1660620-1662142 | 1.37382 | 0.003585 | Uniprot/SPTREMBL:A0A0F7SKR5 |
| CED83400 | Scaffold_52:2189553-2193378 | 1.37211 | 0.038544 | Uniprot/SPTREMBL:A0A0F7SS90 |
| CED84717 | Scaffold_78:1099608-1102777 | 1.36352 | 0.004297 | Uniprot/SPTREMBL:A0A0F7SR63 |
| CED83262 | Scaffold_52:1748245-1750055 | 1.36085 | 0.033623 | Uniprot/SPTREMBL:A0A0F7SMH1 |
| CED85636 | Scaffold_249:2230567-2237697 | 1.35621 | 0.008176 | Uniprot/SPTREMBL:A0A0F7SVP1 |
| CED83271 | Scaffold_52:1774600-1776501 | 1.35608 | 0.00946 | Uniprot/SPTREMBL:A0A0F7SP88 |
| CED84610 | Scaffold_78:800066-802271 | 1.35432 | 0.006352 | Uniprot/SPTREMBL:A0A0F7SVM2 |
| CDZ98422 | Scaffold_262:270958-273739 | 1.35393 | 0.002826 | Uniprot/SPTREMBL:A0A0F7SMV6 |
| CDZ96871 | Scaffold_79:578233-584688 | 1.34599 | 0.036323 | Uniprot/SPTREMBL:A0A0F7SEK9 |
| CDZ97741 | Scaffold_162:423647-425533 | 1.34525 | 0.021426 | Uniprot/SPTREMBL:A0A0F7SFV3 |
| CDZ98291 | Scaffold_242:539694-546572 | 1.33466 | 0.018414 | Uniprot/SPTREMBL:A0A0F7SHB6 |
| CDZ97728 | Scaffold_162:389377-390258 | 1.33175 | 0.04182 | Uniprot/SPTREMBL:A0A0F7SJA1 |
| CDZ98348 | Scaffold_262:54334-55821 | 1.33015 | 0.003585 | Uniprot/SPTREMBL:A0A0F7SL12 |
| CED84009 | Scaffold_69:1435898-1436204 | 1.3291 | 0.019451 | Uniprot/SPTREMBL:A0A0F7STM1 |
| CED85638 | Scaffold_249:2239413-2241638 | 1.32658 | 0.003585 | Uniprot/SPTREMBL:A0A0F7SZ74 |
| CED84849 | Scaffold_78:1487844-1488916 | 1.32316 | 0.044221 | Uniprot/SPTREMBL:A0A0F7SVU9 |
| CDZ98506 | Scaffold_262:521152-522007 | 1.32277 | 0.012761 | Uniprot/SPTREMBL:A0A0F7SHR7 |
| CED82117 | Scaffold_33:352832-354601 | 1.31747 | 0.00697 | Uniprot/SPTREMBL:A0A0F7SJ77 |
| CED83712 | Scaffold_69:556348-559225 | 1.31552 | 0.023063 | Uniprot/SPTREMBL:A0A0F7SNS0 |
| CDZ98726 | Scaffold_262:1216707-1218957 | 1.3071 | 0.004297 | Uniprot/SPTREMBL:A0A0F7SIG5 |
| CED84966 | Scaffold_249:102086-105924 | 1.30617 | 0.008794 | Uniprot/SPTREMBL:A0A0F7SU20 |
| CDZ98007 | Scaffold_189:155162-158020 | 1.3058 | 0.02103 | Uniprot/SPTREMBL:A0A0F7SM57 |
| CED83352 | Scaffold_52:2030450-2032970 | 1.30096 | 0.011005 | Uniprot/SPTREMBL:A0A0F7SMR0 |
| CED83142 | Scaffold_52:1419683-1420879 | 1.29835 | 0.010025 | Uniprot/SPTREMBL:A0A0F7SM39 |
| CED82188 | Scaffold_33:570189-571995 | 1.29177 | 0.008176 | Uniprot/SPTREMBL:A0A0F7SPX6 |
| CDZ96996 | Scaffold_79:956121-957904 | 1.28872 | 0.011005 | Uniprot/SPTREMBL:A0A0F7SER8 |
| CDZ96775 | Scaffold_79:305615-306953 | 1.28852 | 0.044221 | Uniprot/SPTREMBL:A0A0F7SF61 |
| CED82003 | Scaffold_33:6263-8990 | 1.28482 | 0.008176 | Uniprot/SPTREMBL:A0A0F7SPC8 |
| CDZ98578 | Scaffold_262:765711-771979 | 1.28359 | 0.038544 | Uniprot/SPTREMBL:A0A0F7SLK6 |
| CED85353 | Scaffold_249:1337251-1339959 | 1.27446 | 0.025536 | Uniprot/SPTREMBL:A0A0F7SYM4 |
| CDZ98588 | Scaffold_262:796167-798642 | 1.27123 | 0.013896 | Uniprot/SPTREMBL:A0A0F7SLL0 |
| CED83307 | Scaffold_52:1875282-1876355 | 1.27038 | 0.022195 | Uniprot/SPTREMBL:A0A0F7SMM7 |
| CED84032 | Scaffold_69:1512430-1513697 | 1.27023 | 0.021426 | Uniprot/SPTREMBL:A0A0F7SPE1 |
| CDZ98569 | Scaffold_262:739379-741142 | 1.26802 | 0.041478 | Uniprot/SPTREMBL:A0A0F7SM79 |
| CDZ98688 | Scaffold_262:1099242-1100302 | 1.26445 | 0.00697 | Uniprot/SPTREMBL:A0A0F7SLR9 |
| CED83715 | Scaffold_69:562869-565995 | 1.24624 | 0.008794 | Uniprot/SPTREMBL:A0A0F7ST29 |
| CED83820 | Scaffold_69:882993-885014 | 1.24418 | 0.024732 | Uniprot/SPTREMBL:A0A0F7STE7 |
| CED83549 | Scaffold_69:61984-62980 | 1.24031 | 0.042833 | Uniprot/SPTREMBL:A0A0F7SSE4 |
| CED84460 | Scaffold_78:355483-356767 | 1.23011 | 0.023063 | Uniprot/SPTREMBL:A0A0F7SV97 |
| CED85451 | Scaffold_249:1624921-1626657 | 1.22324 | 0.043104 | Uniprot/SPTREMBL:A0A0F7SV85 |
| CED82386 | Scaffold_33:1218622-1221995 | 1.22271 | 0.012761 | Uniprot/SPTREMBL:A0A0F7SLV1 |
| CED83043 | Scaffold_52:1135868-1137875 | 1.22052 | 0.010525 | Uniprot/SPTREMBL:A0A0F7SSK2 |
| CDZ98811 | Scaffold_262:1473304-1476092 | 1.21361 | 0.032587 | Uniprot/SPTREMBL:A0A0F7SIN6 |
| CDZ97506 | Scaffold_124:85143-88051 | 1.20656 | 0.02507 | Uniprot/SPTREMBL:A0A0F7SFD8 |
| CED83086 | Scaffold_52:1254770-1257364 | 1.19723 | 0.014998 | Uniprot/SPTREMBL:A0A0F7SNS9 |
| CED82788 | Scaffold_52:403939-404632 | 1.19422 | 0.037386 | Uniprot/SPTREMBL:A0A0F7SRW9 |
| CED84216 | Scaffold_69:2042437-2043750 | 1.19358 | 0.017391 | Uniprot/SPTREMBL:A0A0F7SRY7 |
| CDZ96433 | Scaffold_54:153007-155386 | 1.1934 | 0.032587 | Uniprot/SPTREMBL:A0A0F7SFP7 |
| CDZ98349 | Scaffold_262:56113-60622 | 1.19098 | 0.016475 | Uniprot/SPTREMBL:A0A0F7SLT6 |
| CED83191 | Scaffold_52:1553706-1554253 | 1.18772 | 0.004297 | Uniprot/SPTREMBL:A0A0F7SP16 |
| CDZ98482 | Scaffold_262:454249-456087 | 1.18613 | 0.028368 | Uniprot/SPTREMBL:A0A0F7SN03 |
| CDZ96583 | Scaffold_54:624919-626843 | 1.18481 | 0.034352 | Uniprot/SPTREMBL:A0A0F7SFW2 |
| CDZ97486 | Scaffold_124:25893-28134 | 1.18431 | 0.020508 | Uniprot/SPTREMBL:A0A0F7SFC9 |
| CED83905 | Scaffold_69:1122672-1126938 | 1.17901 | 0.015553 | Uniprot/SPTREMBL:A0A0F7STK9 |
| CED83752 | Scaffold_69:654878-656794 | 1.17072 | 0.031879 | Uniprot/SPTREMBL:A0A0F7SNU1 |
| CED83251 | Scaffold_52:1716422-1717459 | 1.16668 | 0.038728 | Uniprot/SPTREMBL:A0A0F7SP79 |
| CED83476 | Scaffold_52:2440519-2444040 | 1.16528 | 0.024732 | Uniprot/SPTREMBL:A0A0F7SPZ9 |
| CED83038 | Scaffold_52:1127125-1128716 | 1.16222 | 0.031209 | Uniprot/SPTREMBL:A0A0F7SSJ7 |
| CDZ96921 | Scaffold_79:720825-723066 | 1.15054 | 0.018414 | Uniprot/SPTREMBL:A0A0F7SEP0 |
| CED83405 | Scaffold_52:2204641-2206845 | 1.15036 | 0.014466 | Uniprot/SPTREMBL:A0A0F7SS92 |
| CED85577 | Scaffold_249:2034538-2035696 | 1.14395 | 0.022695 | Uniprot/SPTREMBL:A0A0F7STS4 |
| CED84341 | Scaffold_69:2427694-2431829 | 1.14249 | 0.016101 | Uniprot/SPTREMBL:A0A0F7SS93 |
| CED84378 | Scaffold_78:55256-58433 | 1.14156 | 0.025536 | Uniprot/SPTREMBL:A0A0F7SWD5 |
| CED83553 | Scaffold_69:75264-77681 | 1.14032 | 0.024732 | Uniprot/SPTREMBL:A0A0F7SU77 |
| CED82771 | Scaffold_52:350846-353099 | 1.13824 | 0.033623 | Uniprot/SPTREMBL:A0A0F7SMY7 |
| CDZ97021 | Scaffold_79:1038990-1040784 | 1.13037 | 0.017391 | Uniprot/SPTREMBL:A0A0F7SES3 |
| CED83259 | Scaffold_52:1738331-1743319 | 1.12885 | 0.038544 | Uniprot/SPTREMBL:A0A0F7SR95 |
| CED82412 | Scaffold_33:1307391-1309267 | 1.12475 | 0.016475 | Uniprot/SPTREMBL:A0A0F7SJZ3 |
| CDZ98341 | Scaffold_262:35667-38316 | 1.12283 | 0.028368 | Uniprot/SPTREMBL:A0A0F7SHG1 |
| CED85033 | Scaffold_249:337165-337887 | 1.12149 | 0.019982 | Uniprot/SPTREMBL:A0A0F7SXU0 |
| CED82616 | Scaffold_33:1954259-1956659 | 1.10658 | 0.039325 | Uniprot/SPTREMBL:A0A0F7SMG4 |
| CDZ97069 | Scaffold_79:1183297-1184786 | 1.09255 | 0.029947 | Uniprot/SPTREMBL:A0A0F7SIC3 |
| CED85496 | Scaffold_249:1787393-1789483 | 1.08855 | 0.020508 | Uniprot/SPTREMBL:A0A0F7SVC5 |
| CED83300 | Scaffold_52:1857521-1859414 | 1.08604 | 0.031209 | Uniprot/SPTREMBL:A0A0F7SS27 |
| CDZ98200 | Scaffold_242:250674-254711 | 1.08597 | 0.041478 | Uniprot/SPTREMBL:A0A0F7SIG2 |
| CED84746 | Scaffold_78:1198346-1201827 | 1.0804 | 0.029947 | Uniprot/SPTREMBL:A0A0F7STB9 |
| CED83284 | Scaffold_52:1809160-1812031 | 1.07834 | 0.04884 | Uniprot/SPTREMBL:A0A0F7SRE1 |
| CED84708 | Scaffold_78:1072521-1075060 | 1.07826 | 0.031879 | Uniprot/SPTREMBL:A0A0F7SX29 |
| CED83817 | Scaffold_69:875777-877630 | 1.07691 | 0.02507 | Uniprot/SPTREMBL:A0A0F7SP17 |
| CED83099 | Scaffold_52:1293775-1295496 | 1.06576 | 0.021773 | Uniprot/SPTREMBL:A0A0F7SQW6 |
| CDZ98336 | Scaffold_262:20250-23019 | 1.05966 | 0.037015 | Uniprot/SPTREMBL:A0A0F7SHF8 |
| CED82364 | Scaffold_33:1144383-1147171 | 1.04101 | 0.03518 | Uniprot/SPTREMBL:A0A0F7SNW1 |
| CDZ98117 | Scaffold_217:54-464 | 1.02549 | 0.038728 | Uniprot/SPTREMBL:A0A0F7SMD0 |
| CED83984 | Scaffold_69:1359841-1362644 | 1.01909 | 0.029947 | Uniprot/SPTREMBL:A0A0F7STJ7 |
| CED84925 | Scaffold_78:1735682-1737491 | 1.01862 | 0.038728 | Uniprot/SPTREMBL:A0A0F7SWF4 |
| CED82457 | Scaffold_33:1446468-1449248 | 1.01542 | 0.04182 | Uniprot/SPTREMBL:A0A0F7SK27 |
| CDZ96188 | Scaffold_24:80138-81245 | 1.00373 | 0.04884 | Uniprot/SPTREMBL:A0A0F7SFB4 |
| CED82454 | Scaffold_33:1436004-1437727 | 0.992268 | 0.042098 | Uniprot/SPTREMBL:A0A0F7SP07 |
| CED83808 | Scaffold_69:839010-841550 | −0.978993 | 0.047318 | Uniprot/SPTREMBL:A0A0F7SV12 |
| CED85102 | Scaffold_249:534858-536034 | −0.985732 | 0.041196 | Uniprot/SPTREMBL:A0A0F7SSD5 |
| CDZ97737 | Scaffold_162:414362-415273 | −1.0347 | 0.037015 | Uniprot/SPTREMBL:A0A0F7SLC3 |
| CED83020 | Scaffold_52:1066811-1069140 | −1.03586 | 0.038238 | Uniprot/SPTREMBL:A0A0F7SRA2 |
| CDZ97353 | Scaffold_79:2035798-2037908 | −1.04258 | 0.035564 | Uniprot/SPTREMBL:A0A0F7SI09 |
| CED83590 | Scaffold_69:189208-191677 | −1.04734 | 0.044547 | Uniprot/SPTREMBL:A0A0F7SSR7 |
| CDZ97748 | Scaffold_162:440988-444947 | −1.05847 | 0.046032 | Uniprot/SPTREMBL:A0A0F7SJC1 |
| CDZ98248 | Scaffold_242:405320-407366 | −1.06134 | 0.034795 | Uniprot/SPTREMBL:A0A0F7SKS7 |
| CED82777 | Scaffold_52:366477-369023 | −1.06214 | 0.024433 | Uniprot/SPTREMBL:A0A0F7SKV2 |
| CDZ98789 | Scaffold_262:1417586-1420425 | −1.06443 | 0.02507 | Uniprot/SPTREMBL:A0A0F7SMW9 |
| CED85557 | Scaffold_249:1982109-1984110 | −1.07089 | 0.042435 | Uniprot/SPTREMBL:A0A0F7STQ4 |
| CDZ98271 | Scaffold_242:482272-482866 | −1.09092 | 0.045325 | Uniprot/SPTREMBL:A0A0F7SHA1 |
| CDZ98662 | Scaffold_262:1019159-1020208 | −1.10081 | 0.023509 | Uniprot/SPTREMBL:A0A0F7SNI7 |
| CED82472 | Scaffold_33:1494855-1496930 | −1.10171 | 0.018414 | Uniprot/SPTREMBL:A0A0F7SK39 |
| CDZ97707 | Scaffold_162:338953-341097 | −1.10306 | 0.021773 | Uniprot/SPTREMBL:A0A0F7SL99 |
| CED85444 | Scaffold_249:1598059-1599446 | −1.10671 | 0.024433 | Uniprot/SPTREMBL:A0A0F7SXI2 |
| CDZ96809 | Scaffold_79:406623-408256 | −1.12039 | 0.024732 | Uniprot/SPTREMBL:A0A0F7SHL1 |
| CDZ96362 | Scaffold_53:66709-68840 | −1.12412 | 0.041478 | Uniprot/SPTREMBL:A0A0F7SH50 |
| CDZ97886 | Scaffold_162:851177-854338 | −1.12472 | 0.019451 | Uniprot/SPTREMBL:A0A0F7SG92 |
| CDZ97531 | Scaffold_124:175643-176259 | −1.1447 | 0.011566 | Uniprot/SPTREMBL:A0A0F7SFF4 |
| CDZ97630 | Scaffold_162:88839-89856 | −1.14847 | 0.014998 | Uniprot/SPTREMBL:A0A0F7SGX6 |
| CED85001 | Scaffold_249:223979-224984 | −1.15874 | 0.02507 | Uniprot/SPTREMBL:A0A0F7SU78 |
| CED84248 | Scaffold_69:2142836-2144757 | −1.1588 | 0.021426 | Uniprot/SPTREMBL:A0A0F7SW39 |
| CED84017 | Scaffold_69:1466803-1468329 | −1.16715 | 0.023509 | Uniprot/SPTREMBL:A0A0F7SPD2 |
| CED84592 | Scaffold_78:741601-743464 | −1.17198 | 0.017391 | Uniprot/SPTREMBL:A0A0F7SQU2 |
| CDZ96956 | Scaffold_79:851688-852078 | −1.17483 | 0.032587 | Uniprot/SPTREMBL:A0A0F7SEQ5 |
| CED85092 | Scaffold_249:492482-495573 | −1.17844 | 0.027321 | Uniprot/SPTREMBL:A0A0F7SSC5 |
| CED82255 | Scaffold_33:790676-791081 | −1.18841 | 0.012761 | Uniprot/SPTREMBL:A0A0F7SNS2 |
| CED82283 | Scaffold_33:873009-877325 | −1.19141 | 0.008176 | Uniprot/SPTREMBL:A0A0F7SQ69 |
| CED82196 | Scaffold_33:596179-597979 | −1.19696 | 0.017391 | Uniprot/SPTREMBL:A0A0F7SL59 |
| CED83534 | Scaffold_69:26238-28721 | −1.20197 | 0.014466 | Uniprot/SPTREMBL:A0A0F7SSC9 |
| CDZ97633 | Scaffold_162:95746-97373 | −1.20226 | 0.016937 | Uniprot/SPTREMBL:A0A0F7SIS9 |
| CED83828 | Scaffold_69:912691-913814 | −1.20493 | 0.010025 | Uniprot/SPTREMBL:A0A0F7SV71 |
| CED83695 | Scaffold_69:506810-508045 | −1.20596 | 0.011566 | Uniprot/SPTREMBL:A0A0F7ST13 |
| CED85542 | Scaffold_249:1928334-1933614 | −1.20846 | 0.010525 | Uniprot/SPTREMBL:A0A0F7STP1 |
| CED85129 | Scaffold_249:609328-612469 | −1.23685 | 0.00946 | Uniprot/SPTREMBL:A0A0F7SWG4 |
| CDZ98405 | Scaffold_262:223404-225048 | −1.24291 | 0.02962 | Uniprot/SPTREMBL:A0A0F7SJ27 |
| CED85490 | Scaffold_249:1765942-1769233 | −1.25343 | 0.00697 | Uniprot/SPTREMBL:A0A0F7SY73 |
| CED85278 | Scaffold_249:1098519-1099599 | −1.25754 | 0.011005 | Uniprot/SPTREMBL:A0A0F7SYD5 |
| CDZ98800 | Scaffold_262:1444973-1446444 | −1.26381 | 0.010525 | Uniprot/SPTREMBL:A0A0F7SKG7 |
| CED84979 | Scaffold_249:145173-147662 | −1.26975 | 0.014466 | Uniprot/SPTREMBL:A0A0F7SW36 |
| CED83389 | Scaffold_52:2151630-2154286 | −1.27251 | 0.010525 | Uniprot/SPTREMBL:A0A0F7SRR6 |
| CDZ97840 | Scaffold_162:727586-729218 | −1.27681 | 0.00697 | Uniprot/SPTREMBL:A0A0F7SHH7 |
| CED82710 | Scaffold_52:148247-149050 | −1.27962 | 0.022195 | Uniprot/SPTREMBL:A0A0F7SQ90 |
| CDZ97744 | Scaffold_162:432635-434201 | −1.288 | 0.021773 | Uniprot/SPTREMBL:A0A0F7SK71 |
| CED83517 | Scaffold_52:2551434-2553636 | −1.30585 | 0.010025 | Uniprot/SPTREMBL:A0A0F7SN81 |
| CED83472 | Scaffold_52:2424613-2428401 | −1.31501 | 0.002826 | Uniprot/SPTREMBL:A0A0F7SN42 |
| CDZ98888 | Scaffold_262:1716621-1719877 | −1.31542 | 0.010525 | Uniprot/SPTREMBL:A0A0F7SMT6 |
| CED84417 | Scaffold_78:190965-193843 | −1.31562 | 0.022695 | Uniprot/SPTREMBL:A0A0F7SQC2 |
| CED82856 | Scaffold_52:615050-617722 | −1.34542 | 0.008176 | Uniprot/SPTREMBL:A0A0F7SNB3 |
| CDZ98000 | Scaffold_189:132797-135164 | −1.35451 | 0.010025 | Uniprot/SPTREMBL:A0A0F7SHY2 |
| CED85117 | Scaffold_249:574535-575250 | −1.36204 | 0.021426 | Uniprot/SPTREMBL:A0A0F7SSF0 |
| CED85637 | Scaffold_249:2238001-2238611 | −1.36511 | 0.006352 | Uniprot/SPTREMBL:A0A0F7STX9 |
| CED83010 | Scaffold_52:1037960-1038639 | −1.36959 | 0.005005 | Uniprot/SPTREMBL:A0A0F7SR81 |
| CED84090 | Scaffold_69:1691100-1694027 | −1.3718 | 0.031879 | Uniprot/SPTREMBL:A0A0F7SU51 |
| CED84418 | Scaffold_78:194247-197639 | −1.37256 | 0.016937 | Uniprot/SPTREMBL:A0A0F7SWE3 |
| CDZ97839 | Scaffold_162:723435-726147 | −1.38193 | 0.019982 | Uniprot/SPTREMBL:A0A0F7SKA0 |
| CED85497 | Scaffold_249:1790049-1792770 | −1.38461 | 0.022195 | Uniprot/SPTREMBL:A0A0F7STK2 |
| CED84228 | Scaffold_69:2077031-2079020 | −1.38491 | 0.006352 | Uniprot/SPTREMBL:A0A0F7SW30 |
| CDZ98142 | Scaffold_242:72315-72984 | −1.39076 | 0.016475 | Uniprot/SPTREMBL:A0A0F7SMF1 |
| CED83841 | Scaffold_69:945530-947189 | −1.3942 | 0.003585 | Uniprot/SPTREMBL:A0A0F7SR18 |
| CED84830 | Scaffold_78:1431276-1433662 | −1.41737 | 0.001074 | Uniprot/SPTREMBL:A0A0F7SW68 |
| CDZ96750 | Scaffold_79:233801-235070 | −1.41946 | 0.005005 | Uniprot/SPTREMBL:A0A0F7SF52 |
| CDZ98868 | Scaffold_262:1656053-1656973 | −1.42059 | 0.001074 | Uniprot/SPTREMBL:A0A0F7SMR8 |
| CDZ97870 | Scaffold_162:808063-808921 | −1.42078 | 0.028707 | Uniprot/SPTREMBL:A0A0F7SHK3 |
| CED84081 | Scaffold_69:1660757-1661605 | −1.42877 | 0.010525 | Uniprot/SPTREMBL:A0A0F7SRM1 |
| CED84218 | Scaffold_69:2047542-2048755 | −1.43718 | 0.001074 | Uniprot/SPTREMBL:A0A0F7SW26 |
| CED84514 | Scaffold_78:515920-517461 | −1.44083 | 0.005005 | Uniprot/SPTREMBL:A0A0F7SUW1 |
| CED84940 | Scaffold_249:36361-39079 | −1.44153 | 0.006352 | Uniprot/SPTREMBL:A0A0F7SWG3 |
| CDZ96815 | Scaffold_79:422815-425938 | −1.44247 | 0.001074 | Uniprot/SPTREMBL:A0A0F7SF76 |
| CED82015 | Scaffold_33:46454-47993 | −1.44841 | 0.001074 | Uniprot/SPTREMBL:A0A0F7SN44 |
| CED84369 | Scaffold_78:29525-30507 | −1.45503 | 0.003585 | Uniprot/SPTREMBL:A0A0F7SUL5 |
| CED83696 | Scaffold_69:511061-511998 | −1.46363 | 0.011005 | Uniprot/SPTREMBL:A0A0F7SQJ0 |
| CED85556 | Scaffold_249:1975453-1981704 | −1.47673 | 0.016475 | Uniprot/SPTREMBL:A0A0F7SVI0 |
| CED82505 | Scaffold_33:1609241-1610829 | −1.48428 | 0.032989 | Uniprot/SPTREMBL:A0A0F7SPT3 |
| CED82697 | Scaffold_52:107314-110474 | −1.4861 | 0.001074 | Uniprot/SPTREMBL:A0A0F7SKM2 |
| CDZ96174 | Scaffold_24:45029-46448 | −1.48792 | 0.001998 | Uniprot/SPTREMBL:A0A0F7SFZ4 |
| CDZ97929 | Scaffold_162:985527-987486 | −1.49375 | 0.001074 | Uniprot/SPTREMBL:A0A0F7SKI7 |
| CED83303 | Scaffold_52:1864569-1867964 | −1.49629 | 0.001998 | Uniprot/SPTREMBL:A0A0F7STG3 |
| CDZ96727 | Scaffold_79:168408-169040 | −1.50767 | 0.035564 | Uniprot/SPTREMBL:A0A0F7SID4 |
| CDZ98383 | Scaffold_262:163269-165707 | −1.50983 | 0.001998 | Uniprot/SPTREMBL:A0A0F7SL66 |
| CDZ97208 | Scaffold_79:1602896-1603746 | −1.52051 | 0.028024 | Uniprot/SPTREMBL:A0A0F7SHL4 |
| CED84696 | Scaffold_78:1041262-1043489 | −1.52121 | 0.005005 | Uniprot/SPTREMBL:A0A0F7ST73 |
| CED85179 | Scaffold_249:768057-769128 | −1.528 | 0.005005 | Uniprot/SPTREMBL:A0A0F7SWN5 |
| CDZ97456 | Scaffold_79:2325450-2327139 | −1.53518 | 0.001998 | Uniprot/SPTREMBL:A0A0F7SFB7 |
| CED84729 | Scaffold_78:1142277-1143722 | −1.54842 | 0.02507 | Uniprot/SPTREMBL:A0A0F7SVE0 |
| CDZ97944 | Scaffold_162:1029594-1031564 | −1.55315 | 0.032288 | Uniprot/SPTREMBL:A0A0F7SKJ3 |
| CED82961 | Scaffold_52:899381-901807 | −1.55439 | 0.001998 | Uniprot/SPTREMBL:A0A0F7SNJ6 |
| CED85145 | Scaffold_249:663696-666391 | −1.56549 | 0.001074 | Uniprot/SPTREMBL:A0A0F7SWW3 |
| CDZ96993 | Scaffold_79:946175-946791 | −1.56735 | 0.010025 | Uniprot/SPTREMBL:A0A0F7SH00 |
| CDZ98218 | Scaffold_242:313958-314979 | −1.57462 | 0.039739 | Uniprot/SPTREMBL:A0A0F7SKP7 |
| CDZ97354 | Scaffold_79:2038247-2039550 | −1.57466 | 0.026366 | Uniprot/SPTREMBL:A0A0F7SJ53 |
| CED85074 | Scaffold_249:442737-444170 | −1.58124 | 0.001074 | Uniprot/SPTREMBL:A0A0F7SWB7 |
| CDZ97526 | Scaffold_124:162119-164466 | −1.5836 | 0.001074 | Uniprot/SPTREMBL:A0A0F7SFF1 |
| CDZ98135 | Scaffold_242:49823-52376 | −1.58729 | 0.001074 | Uniprot/SPTREMBL:A0A0F7SI82 |
| CED84431 | Scaffold_78:237377-238586 | −1.60776 | 0.001074 | Uniprot/SPTREMBL:A0A0F7SSJ4 |
| CED85313 | Scaffold_249:1208655-1213573 | −1.63218 | 0.004297 | Uniprot/SPTREMBL:A0A0F7SYJ8 |
| CED84836 | Scaffold_78:1448687-1449940 | −1.63345 | 0.023995 | Uniprot/SPTREMBL:A0A0F7STM7 |
| CED83274 | Scaffold_52:1784640-1786631 | −1.66122 | 0.022195 | Uniprot/SPTREMBL:A0A0F7SRA0 |
| CED85626 | Scaffold_249:2189998-2193215 | −1.6636 | 0.001074 | Uniprot/SPTREMBL:A0A0F7SVN2 |
| CDZ97245 | Scaffold_79:1727581-1730708 | −1.66565 | 0.015553 | Uniprot/SPTREMBL:A0A0F7SFX9 |
| CED85026 | Scaffold_249:319340-320235 | −1.6759 | 0.001074 | Uniprot/SPTREMBL:A0A0F7SU90 |
| CDZ97907 | Scaffold_162:926229-926777 | −1.68467 | 0.001074 | Uniprot/SPTREMBL:A0A0F7SLM2 |
| CED85146 | Scaffold_249:667273-668861 | −1.6897 | 0.001074 | Uniprot/SPTREMBL:A0A0F7SUH6 |
| CDZ96446 | Scaffold_54:193458-195288 | −1.69218 | 0.005005 | Uniprot/SPTREMBL:A0A0F7SE96 |
| CED85386 | Scaffold_249:1428350-1429974 | −1.69704 | 0.007631 | Uniprot/SPTREMBL:A0A0F7SV36 |
| CED83236 | Scaffold_52:1660428-1665437 | −1.70904 | 0.019451 | Uniprot/SPTREMBL:A0A0F7SP66 |
| CDZ97306 | Scaffold_79:1907876-1908952 | −1.71442 | 0.001074 | Uniprot/SPTREMBL:A0A0F7SF29 |
| CED83823 | Scaffold_69:895697-898455 | −1.71756 | 0.007631 | Uniprot/SPTREMBL:A0A0F7SV28 |
| CDZ97777 | Scaffold_162:521349-522266 | −1.7263 | 0.001074 | Uniprot/SPTREMBL:A0A0F7SLD7 |
| CED85594 | Scaffold_249:2084742-2086107 | −1.73902 | 0.001074 | Uniprot/SPTREMBL:A0A0F7SXV3 |
| CDZ97012 | Scaffold_79:1010624-1012290 | −1.74477 | 0.017391 | Uniprot/SPTREMBL:A0A0F7SJ45 |
| CED85303 | Scaffold_249:1175266-1177190 | −1.75298 | 0.030842 | Uniprot/SPTREMBL:A0A0F7SYJ0 |
| CDZ98236 | Scaffold_242:365011-366553 | −1.76023 | 0.001074 | Uniprot/SPTREMBL:A0A0F7SH75 |
| CDZ96540 | Scaffold_54:497380-497756 | −1.77148 | 0.001074 | Uniprot/SPTREMBL:A0A0F7SEX6 |
| CDZ96250 | Scaffold_24:287314-287702 | −1.78589 | 0.025999 | Uniprot/SPTREMBL:A0A0F7SEK1 |
| CED82279 | Scaffold_33:855341-858413 | −1.78693 | 0.001074 | Uniprot/SPTREMBL:A0A0F7SNL5 |
| CDZ96876 | Scaffold_79:595587-596170 | −1.79528 | 0.005005 | Uniprot/SPTREMBL:A0A0F7SEL1 |
| CED83529 | Scaffold_69:6140-10274 | −1.79593 | 0.013319 | Uniprot/SPTREMBL:A0A0F7SSA8 |
| CDZ98664 | Scaffold_262:1024362-1027941 | −1.79806 | 0.043499 | Uniprot/SPTREMBL:A0A0F7SMD3 |
| CED83336 | Scaffold_52:1970924-1973100 | −1.84478 | 0.001074 | Uniprot/SPTREMBL:A0A0F7SPJ6 |
| CDZ98496 | Scaffold_262:495689-498598 | −1.86631 | 0.001998 | Uniprot/SPTREMBL:A0A0F7SHQ9 |
| CED83987 | Scaffold_69:1368003-1369177 | −1.87504 | 0.001074 | Uniprot/SPTREMBL:A0A0F7SPB6 |
| CED82860 | Scaffold_52:629851-631596 | −1.8768 | 0.001074 | Uniprot/SPTREMBL:A0A0F7SQU0 |
| CED84129 | Scaffold_69:1804647-1807171 | −1.89497 | 0.001074 | Uniprot/SPTREMBL:A0A0F7STY0 |
| CDZ96983 | Scaffold_79:922379-925116 | −1.90977 | 0.004297 | Uniprot/SPTREMBL:A0A0F7SGZ2 |
| CED83784 | Scaffold_69:745510-748575 | −1.91551 | 0.001998 | Uniprot/SPTREMBL:A0A0F7ST12 |
| CED82708 | Scaffold_52:141033-144586 | −1.92185 | 0.001074 | Uniprot/SPTREMBL:A0A0F7SRK5 |
| CED84035 | Scaffold_69:1521641-1522601 | −1.947 | 0.026366 | Uniprot/SPTREMBL:A0A0F7STX6 |
| CED83223 | Scaffold_52:1629817-1630351 | −1.95328 | 0.001074 | Uniprot/SPTREMBL:A0A0F7ST26 |
| CED85344 | Scaffold_249:1300710-1302755 | −1.95401 | 0.001074 | Uniprot/SPTREMBL:A0A0F7SXB7 |
| CED83617 | Scaffold_69:267842-271966 | −1.96243 | 0.001074 | Uniprot/SPTREMBL:A0A0F7SNK8 |
| CDZ96955 | Scaffold_79:850815-851296 | −1.96811 | 0.011005 | Uniprot/SPTREMBL:A0A0F7SFD0 |
| CDZ96589 | Scaffold_54:643479-645853 | −1.98651 | 0.001074 | Uniprot/SPTREMBL:A0A0F7SGX4 |
| CDZ97148 | Scaffold_79:1418833-1420884 | −1.98756 | 0.012168 | Uniprot/SPTREMBL:A0A0F7SHA7 |
| CED83678 | Scaffold_69:454981-456676 | −2.00538 | 0.013896 | Uniprot/SPTREMBL:A0A0F7SUM1 |
| CDZ97063 | Scaffold_79:1160642-1161469 | −2.00563 | 0.001074 | Uniprot/SPTREMBL:A0A0F7SH47 |
| CED83232 | Scaffold_52:1650347-1652499 | −2.00736 | 0.019451 | Uniprot/SPTREMBL:A0A0F7SME3 |
| CED84569 | Scaffold_78:683193-684172 | −2.01311 | 0.038728 | Uniprot/SPTREMBL:A0A0F7SUZ6 |
| CED85593 | Scaffold_249:2082233-2084415 | −2.0274 | 0.001074 | Uniprot/SPTREMBL:A0A0F7SZ46 |
| CED82571 | Scaffold_33:1819188-1821941 | −2.03204 | 0.001074 | Uniprot/SPTREMBL:A0A0F7SMA6 |
| CED82024 | Scaffold_33:79365-79988 | −2.03815 | 0.001074 | Uniprot/SPTREMBL:A0A0F7SMX9 |
| CED84840 | Scaffold_78:1463247-1464601 | −2.04553 | 0.001074 | Uniprot/SPTREMBL:O60019 |
| CDZ97958 | Scaffold_185:2335-3721 | −2.05558 | 0.001074 | Uniprot/SPTREMBL:A0A0F7SJV4 |
| CDZ96695 | Scaffold_79:79818-82579 | −2.05792 | 0.001074 | Uniprot/SPTREMBL:A0A0F7SF31 |
| CED84039 | Scaffold_69:1530094-1533993 | −2.06613 | 0.001074 | Uniprot/SPTREMBL:A0A0F7STP4 |
| CED83377 | Scaffold_52:2117808-2120026 | −2.08523 | 0.001074 | Uniprot/SPTREMBL:A0A0F7SMT0 |
| CED82529 | Scaffold_33:1688676-1690831 | −2.1036 | 0.010525 | Uniprot/SPTREMBL:A0A0F7SP73 |
| CDZ97994 | Scaffold_189:112542-113046 | −2.11032 | 0.001074 | Uniprot/SPTREMBL:A0A0F7SKM4 |
| CED83890 | Scaffold_69:1080480-1082192 | −2.12819 | 0.001074 | Uniprot/SPTREMBL:A0A0F7STJ5 |
| CED82578 | Scaffold_33:1844646-1846846 | −2.14622 | 0.001074 | Uniprot/SPTREMBL:A0A0F7SR99 |
| CDZ98694 | Scaffold_262:1113499-1115533 | −2.17485 | 0.001074 | Uniprot/SPTREMBL:A0A0F7SMM3 |
| CED84398 | Scaffold_78:130805-133471 | −2.17595 | 0.001074 | Uniprot/SPTREMBL:A0A0F7SWD9 |
| CED85532 | Scaffold_249:1894027-1894929 | −2.22038 | 0.001074 | Uniprot/SPTREMBL:A0A0F7STN3 |
| CDZ96641 | Scaffold_77:26787-28432 | −2.23969 | 0.001074 | Uniprot/SPTREMBL:A0A0F7SED5 |
| CED85075 | Scaffold_249:444854-448105 | −2.24815 | 0.001074 | Uniprot/SPTREMBL:A0A0F7SWN2 |
| CDZ96176 | Scaffold_24:47894-49932 | −2.26781 | 0.037813 | Uniprot/SPTREMBL:A0A0F7SE42 |
| CDZ96699 | Scaffold_79:90772-94522 | −2.27624 | 0.001074 | Uniprot/SPTREMBL:A0A0F7SHB2 |
| CED82661 | Scaffold_33:2081366-2082099 | −2.29285 | 0.042098 | Uniprot/SPTREMBL:A0A0F7SMJ8 |
| CED85015 | Scaffold_249:277754-280328 | −2.2993 | 0.001074 | Uniprot/SPTREMBL:A0A0F7SWK9 |
| CDZ97249 | Scaffold_79:1746839-1748488 | −2.3182 | 0.001074 | Uniprot/SPTREMBL:A0A0F7SIX6 |
| CDZ96334 | Scaffold_47:23-661 | −2.32877 | 0.001074 | Uniprot/SPTREMBL:A0A0F7SGD2 |
| CED83532 | Scaffold_69:20326-23031 | −2.35024 | 0.001074 | Uniprot/SPTREMBL:A0A0F7SN95 |
| CED84492 | Scaffold_78:450665-451017 | −2.36775 | 0.001074 | Uniprot/SPTREMBL:A0A0F7SQK1 |
| CED85166 | Scaffold_249:719426-720769 | −2.37977 | 0.001074 | Uniprot/SPTREMBL:A0A0F7SUI9 |
| CED83489 | Scaffold_52:2474332-2475671 | −2.38064 | 0.001074 | Uniprot/SPTREMBL:A0A0F7SS48 |
| CDZ96278 | Scaffold_24:368207-369197 | −2.39138 | 0.002826 | Uniprot/SPTREMBL:A0A0F7SFI5 |
| CED84034 | Scaffold_69:1519055-1520738 | −2.4005 | 0.001074 | Uniprot/SPTREMBL:A0A0F7STP0 |
| CED84697 | Scaffold_78:1044388-1045081 | −2.40872 | 0.001998 | Uniprot/SPTREMBL:A0A0F7SR45 |
| CDZ97983 | Scaffold_189:81800-82783 | −2.41732 | 0.001074 | Uniprot/SPTREMBL:A0A0F7SJX3 |
| CDZ96933 | Scaffold_79:768574-769075 | −2.44241 | 0.001074 | Uniprot/SPTREMBL:A0A0F7SGQ8 |
| CDZ98807 | Scaffold_262:1460878-1462217 | −2.46814 | 0.001998 | Uniprot/SPTREMBL:A0A0F7SP56 |
| CED84065 | Scaffold_69:1612575-1613807 | −2.48264 | 0.001074 | Uniprot/SPTREMBL:A0A0F7SU21 |
| CDZ98091 | Scaffold_189:408922-411088 | −2.49712 | 0.001074 | Uniprot/SPTREMBL:A0A0F7SGR3 |
| CED85462 | Scaffold_249:1676632-1679003 | −2.50553 | 0.001074 | Uniprot/SPTREMBL:A0A0F7STH0 |
| CDZ97924 | Scaffold_162:971572-972091 | −2.53677 | 0.001074 | Uniprot/SPTREMBL:A0A0F7SKI4 |
| CDZ97392 | Scaffold_79:2152424-2153342 | −2.55979 | 0.001074 | Uniprot/SPTREMBL:A0A0F7SKD9 |
| CED84952 | Scaffold_249:70148-71328 | −2.57142 | 0.001074 | Uniprot/SPTREMBL:A0A0F7SRU8 |
| CED83368 | Scaffold_52:2087748-2092282 | −2.61347 | 0.001074 | Uniprot/SPTREMBL:A0A0F7STR3 |
| CED83646 | Scaffold_69:350868-353990 | −2.66544 | 0.001074 | Uniprot/SPTREMBL:A0A0F7SQG8 |
| CED85401 | Scaffold_249:1467760-1469049 | −2.69341 | 0.001074 | Uniprot/SPTREMBL:A0A0F7SV47 |
| CED82304 | Scaffold_33:956694-958264 | −2.71132 | 0.001074 | Uniprot/SPTREMBL:A0A0F7SNT3 |
| CDZ97780 | Scaffold_162:527493-528752 | −2.73943 | 0.001074 | Uniprot/SPTREMBL:A0A0F7SHC9 |
| CED82230 | Scaffold_33:706033-706327 | −2.74682 | 0.001074 | Uniprot/SPTREMBL:A0A0F7SNQ6 |
| CED84839 | Scaffold_78:1459933-1461957 | −2.76513 | 0.001074 | Uniprot/SPTREMBL:A0A0F7SVU5 |
| CDZ97703 | Scaffold_162:322608-324574 | −2.77756 | 0.002826 | Uniprot/SPTREMBL:A0A0F7SJ79 |
| CED84019 | Scaffold_69:1470201-1473044 | −2.78186 | 0.001074 | Uniprot/SPTREMBL:A0A0F7STM8 |
| CED85116 | Scaffold_249:571977-573388 | −2.84413 | 0.001074 | Uniprot/SPTREMBL:A0A0F7SUE0 |
| CDZ98221 | Scaffold_242:318841-320089 | −2.86547 | 0.001074 | Uniprot/SPTREMBL:A0A0F7SH67 |
| CDZ96418 | Scaffold_54:107801-108657 | −2.90528 | 0.001074 | Uniprot/SPTREMBL:A0A0F7SFN9 |
| CED82158 | Scaffold_33:482712-485332 | −2.96302 | 0.001074 | Uniprot/SPTREMBL:A0A0F7SPV3 |
| CED83626 | Scaffold_69:289862-292855 | −3.0158 | 0.001074 | Uniprot/SPTREMBL:A0A0F7SQF3 |
| CED82286 | Scaffold_33:885651-888286 | −3.13743 | 0.001074 | Uniprot/SPTREMBL:A0A0F7SLG8 |
| CED82646 | Scaffold_33:2036969-2038849 | −3.14718 | 0.001074 | Uniprot/SPTREMBL:A0A0F7SMJ2 |
| CED82645 | Scaffold_33:2031954-2035008 | −3.22499 | 0.001074 | Uniprot/SPTREMBL:A0A0F7SQ63 |
| CED82966 | Scaffold_52:916495-918046 | −3.27009 | 0.001074 | Uniprot/SPTREMBL:A0A0F7SNJ9 |
| CDZ98220 | Scaffold_242:316107-317206 | −3.33816 | 0.001074 | Uniprot/SPTREMBL:A0A0F7SII2 |
| CDZ97624 | Scaffold_162:66225-66878 | −3.36931 | 0.001074 | Uniprot/SPTREMBL:A0A0F7SJS3 |
| CED83962 | Scaffold_69:1288283-1292950 | −3.46187 | 0.003585 | Uniprot/SPTREMBL:A0A0F7SPA4 |
| CDZ96670 | Scaffold_77:106964-110092 | −3.50921 | 0.001074 | Uniprot/SPTREMBL:A0A0F7SF12 |
| CDZ96436 | Scaffold_54:160437-161831 | −3.66011 | 0.001074 | Uniprot/SPTREMBL:A0A0F7SE94 |
| CED85434 | Scaffold_249:1567428-1569423 | −3.74639 | 0.001074 | Uniprot/SPTREMBL:A0A0F7SXH6 |
| CDZ96388 | Scaffold_54:18689-18947 | −3.88549 | 0.001074 | Uniprot/SPTREMBL:A0A0F7SFM4 |
| CDZ96819 | Scaffold_79:433020-434119 | −4.1179 | 0.001074 | Uniprot/SPTREMBL:A0A0F7SHM0 |
| CDZ96258 | Scaffold_24:318573-319834 | −4.2954 | 0.001074 | Uniprot/SPTREMBL:A0A0F7SFH3 |
| CED83073 | Scaffold_52:1224482-1226236 | −4.39164 | 0.001074 | Uniprot/SPTREMBL:A0A0F7SSN1 |
| CDZ97363 | Scaffold_79:2059802-2060236 | −4.42159 | 0.014998 | Uniprot/SPTREMBL:A0A0F7SI17 |
| CDZ96973 | Scaffold_79:896164-897185 | −4.44277 | 0.001074 | Uniprot/SPTREMBL:A0A0F7SGV4 |
| CED84060 | Scaffold_69:1592326-1594451 | −4.5001 | 0.001074 | Uniprot/SPTREMBL:A0A0F7SU17 |
| CDZ98154 | Scaffold_242:109579-111004 | −4.58439 | 0.001074 | Uniprot/SPTREMBL:A0A0F7SL35 |
| CED82980 | Scaffold_52:954553-957016 | −4.59855 | 0.001074 | Uniprot/SPTREMBL:A0A0F7SR47 |
| CED85062 | Scaffold_249:401979-403145 | −4.85533 | 0.001074 | Uniprot/SPTREMBL:A0A0F7SS57 |

TABLE 17

| | Enzyme name | Gene ID | Log₂ change | q-value | Significant |
|---|---|---|---|---|---|
| T₁ | Glucose-6-phosphate 1-dehydrogenase | CED83429 | −0.42 | 0.6681 | no |
| T₂ | Phosphoglucomutase/phosphomannomutase | CDZ97253 | −0.74 | 0.2877 | no |
| T₃ | 6-phosphofructokinase | CDZ97071 | −1.04 | 0.2238 | no |
| T₄ | Glucose-6-phosphate isomerase | CED83630 | −1.43 | 0.1038 | no |
| T₅ | Fructose 1,6-bisphosphate aldolase | CDZ98367 | 0 | 0.9982 | no |
| T₆ | Phosphoglycerate mutase | CED83840 | −0.79 | 0.1508 | no |
| T₇ | Phosphoglycerate kinase | CDZ96270 | −0.96 | 0.3434 | no |
| T₈ | Glyceraldehyde-3-phosphate dehydrogenase | CED85376 | −0.5 | 0.8098 | no |
| T₉ | Enolase | CED83362 | −0.51 | 0.7568 | no |
| T₁₀ | Pyruvate kinase | CDZ98564 | −0.87 | 0.398 | no |
| T₁₁ | Pyruvate decarboxylase | CED83333 | −1.68 | 0.0746 | no |
| T₁₂ | Isocitrate lyase | CED85129 | −1.24 | 0.0095 | yes |
| T₁₃ | Citrate synthase | CED84246 | 0.71 | 0.2059 | no |
| T₁₄ | Aconitate hydratase | CED85427 | −0.18 | 0.8799 | no |
| T₁₅ | Ketoglutarate dehydrogenase | CED83799 | 1.55 | 0.0156 | yes |
| T₁₆ | Succinate dehydroganase | CDZ98193 | 2.8 | 0.0011 | yes |
| T₁₇ | Malate synthase | CED85130 | −0.14 | 0.8949 | no |
| T₁₈ | 6-phosphogluconate decarboxylating | CED82777 | −1.06 | 0.0244 | yes |
| T₁₉ | Gluconate kinase | CDZ97633 | −1.2 | 0.0169 | yes |
| T₂₀ | Geranylgeranyl pyrophosphate synthase | CED82684 | 0.86 | 0.4365 | no |
| T₂₁ | Phytoene dehydrogenase | CED83513 | 1.8 | 0.2754 | no |
| T₂₂ | Phytoene-beta carotene synthase | CED83449 | 1.52 | 0.087 | no |
| T₂₃ | Astaxanthin synthase | CED83940 | 2.23 | 0.0057 | yes |
| T₂₄ | Cytochrome P450 CYP3/CYP5/CYP6/CYP9 subfamilies | CED84998 | 4.05 | 0.0011 | yes |
| T₂₅ | Cytochrome P450 CYP4/CYP19/CYP26 subfamilies | CDZ98632 | 1.44 | 0.007 | yes |
| T₂₆ | Cytochrome P450 CYP2 subfamily | CED85015 | −2.3 | 0.0011 | yes |
| T₂₇ | Apocytochrome b | CED80059 | 5.32 | 0.0011 | yes |
| T₂₈ | Cytochrome b | CED80058 | 5.15 | 0.0011 | yes |
| T₂₉ | NADH: ubiquinone/plastoquinone oxidoreductase, chain 3 | CDZ96154 | 4.73 | 0.0043 | yes |
| T₃₀ | NADH: ubiquinone oxidoreductase, NDUFA9/39 kDa subunit | CED84925 | 1.02 | 0.0387 | yes |
| T₃₁ | Cytochrome c oxidase subunit 1 | CED80056 | 5.57 | 0.0011 | yes |
| T₃₂ | Cytochrome c oxidase subunit 2 | CDZ96152 | 5.3 | 0.0011 | yes |
| T₃₃ | Cytochrome c oxidase subunit 3 | CED80061 | 5.01 | 0.0011 | yes |
| T₃₄ | Cytochrome c oxidase assembly protein PET191 | CED84572 | 1.67 | 0.0393 | yes |
| T₃₅ | Putative cytochrome c oxidase, subunit COX19 | CED82283 | −1.19 | 0.0082 | yes |
| T₃₆ | ATP synthase subunit 6 | CDZ96150 | 5.25 | 0.0011 | yes |
| T₃₇ | ATP synthase subunit mitochondrial | CDZ96333 | 4.65 | 0.0011 | yes |
| T₃₈ | NADH dehydrogenase subunits 2, 5, and related proteins | CDZ96153 | 6.01 | 0.0011 | yes |
| T₃₉ | NADH dehydrogenase subunit 4 | CDZ96151 | 4.13 | 0.0011 | yes |
| T₄₀ | Aldo/keto reductase family proteins | CDZ97194 | 1.85 | 0.0043 | yes |
| T₄₁ | Aldo/keto reductase family proteins | CDZ97021 | 1.13 | 0.0174 | yes |
| T₄₂ | Hexose carrier protein | CED82529 | −2.1 | 0.0105 | yes |
| T₄₃ | Hexokinase | CED82858 | −1.3 | 0.1658 | no |
| T₄₄ | Ferredoxin/adrenodoxin reductase | CDZ98521 | 1.87 | 0.0438 | yes |
| T₄₅ | acyl-dehydrogenase | CED84717 | 1.36 | 0.0042 | yes |
| T₄₆ | hexose carrier protein | CED82529 | −2.1 | 0.0105 | yes |
| T₄₇ | Catalase 1 | CDZ96425 | 3.16 | 0.0057 | yes |
| T₄₈ | Catalase-like domain | CDZ98863 | 1.53 | 0.0149 | yes |
| T₄₉ | Glutathione S-transferase | CED84930 | 6.68 | 0.001 | yes |
| T₅₀ | Glutathione S-transferase | CDZ97957 | 5.4 | 0.001 | yes |
| T₅₁ | Predicted glutathione S-transferase | CED82196 | −1.19 | 0.0173 | yes |
| T₅₂ | Delta 9 fatty acid desaturase | CED83656 | −0.53 | 0.7699 | no |
| T₅₃ | Asparagine synthase | CED83843 | 2.35 | 0.0081 | yes |
| T₅₄ | Copper transporter | CED82663 | 2.36 | 0.001 | yes |
| T₅₅ | Conidiation-specific protein 6 | CDZ97534 | 3.17 | 0.001 | yes |

TABLE 18

| Protein | log2FC | adj. pvalue | Protein name |
|---|---|---|---|
| CDZ96711 | 3.272326443 | 0.00057316 | short-chain dehydrogenase |
| CED82658 | 2.557045495 | 0.01154058 | Deoxyribodipyrimidine photolyase/cryptochrome |
| CED84367 | 2.318616444 | 0.01799604 | Nuclear transport factor 2 |
| CED85066 | 2.239020548 | 0.03538521 | 60s ribosomal protein I32 |
| CED82723 | 2.140206091 | 0.01820556 | Armadillo/beta-catenin-like repeat-containing protein |
| CDZ96346 | 2.1328695 | 0.02431585 | 60s ribosomal protein I10a |
| CED83196 | 2.114885867 | 0.00057316 | Pyridoxamine 5′-phosphate oxidase-like, FMN-binding domain |
| CDZ97688 | 1.962719758 | 0.0472272 | glutaredoxin-related protein |
| CED82344 | 1.930631149 | 0.01505998 | Glycosyl transferase, family 8-glycogenin |
| CDZ96731 | 1.92915995 | 0.0156358 | mitochondrial carrier |
| CED85501 | 1.799809195 | 0.02428145 | nucleosome assembly protein |
| CED85055 | 1.731193901 | 0.03932151 | Sterile alpha motif, type 2 |
| CED84052 | 1.690858303 | 0.00057316 | snare protein ykt6 |
| CED82753 | 1.672583239 | 0.0472272 | udp-glucose dehydrogenase |
| CED82004 | 1.648999686 | 0.0188681 | Predicted translation factor, contains W2 domain |
| CED82942 | 1.626342108 | 0.0195415 | G protein beta subunit-like protein |
| CED82433 | 1.618966359 | 0.01109532 | heat shock protein hss1 |
| CED84880 | 1.610275821 | 0.03376189 | 40s ribosomal protein s7 |
| CED83740 | 1.589899371 | 0.00905229 | atp synthase f1 beta subunit |
| CED82360 | 1.561433152 | 0.01276031 | hypothetical protein |
| CDZ96425 | 1.552015184 | 0.01060517 | catalase 1 |
| CED84965 | 1.534893848 | 0.04391875 | Stress responsive alpha-beta barrel |
| CED83216 | 1.48274394 | 0.01649435 | Uncharacterized conserved protein |
| CED85079 | 1.452711928 | 0.03416979 | Cytokinin riboside 5′-monophosphate phosphoribohydrolase LOG |
| CDZ96256 | 1.400081003 | 0.01652963 | EF-hand domain pair |
| CED84618 | 1.363223801 | 0.01583624 | 20s proteasome subunit |
| CDZ96583 | 1.336737341 | 0.0020292 | ferrochelatase |
| CDZ98540 | 1.304088911 | 0.03416979 | glycine hydroxymethyltransferase |
| CDZ98291 | 1.299250675 | 0.03376189 | carboxypeptidase s |
| CED84995 | 1.27866734 | 0.0020292 | nadh-ubiquinone oxidoreductase 304 kda subunit precursor |
| CED83513 | 1.272006068 | 0.00427065 | phytoene dehydrogenase |
| CED82558 | 1.255848347 | 0.03519539 | Ribosomal protein L49/IMG2 |
| CED85248 | 1.21214568 | 0.03416979 | nop10p-domain-containing protein |
| CED84223 | 1.211570639 | 0.03297342 | Thioredoxin/protein disulfide isomerase |
| CDZ96398 | 1.210395736 | 0.01697913 | Predicted dehydrogenase |
| CED82081 | 1.124637661 | 0.03043844 | 6-phosphogluconate dehydrogenase |
| CDZ97938 | 1.090327535 | 0.01799604 | NADH-dehydrogenase (ubiquinone) |
| CDZ97188 | 1.041653101 | 0.02454864 | COPII vesicle protein |
| CED82583 | 0.972812085 | 0.04622283 | ornithine aminotransferase |
| CDZ97607 | 0.961343748 | 0.02264306 | er-associated protein catabolism-related protein |
| CDZ98576 | 0.956641899 | 0.04101412 | isocitrate dehydrogenase |
| CED83153 | 0.937690879 | 0.00164427 | aaa atpase |
| CED83708 | 0.919968342 | 0.02513695 | probable nadp( )-dependent dehydrogenase acting on 3-hydroxy acids |
| CED82933 | 0.915744964 | 0.00468473 | cndp dipeptidase |
| CED83441 | 0.899886386 | 0.01389815 | Actin-related protein Arp2/3 complex, subunit ARPC2 |
| CED84012 | 0.882703364 | 0.0396396 | branched-chain amino acid aminotransferase ii |
| CED85564 | 0.878847461 | 0.03403724 | Carbon-nitrogen hydrolase |
| CED84537 | 0.86940849 | 0.04899403 | aspartate aminotransferase |
| CED84732 | 0.819059765 | 0.01820556 | NADPH oxidase |
| CDZ96582 | 0.783460677 | 0.00757621 | 26s proteasome subunit p45 |
| CED84295 | 0.727101959 | 0.04595691 | pre-mrna-splicing factor rse1 |
| CDZ97585 | 0.702232279 | 0.04441472 | Porphobilinogen deaminase |
| CDZ97299 | 0.684300182 | 0.02856417 | prolyl oligopeptidase |
| CED83351 | 0.53279685 | 0.01820556 | abc transporter |
| CED83447 | −0.540843796 | 0.04054781 | 40s ribosomal protein s9 |
| CED82282 | −0.549026515 | 0.02203629 | polyadenylate-binding protein |
| CED83463 | −0.549306874 | 0.02914553 | atp-dependent rna helicase dhx8 |
| CDZ96620 | −0.550699218 | 0.00730273 | Fatty acid synthase complex subunit alpha |
| CED85357 | −0.552976694 | 0.03416979 | glycosyltransferase family 35 protein |
| CDZ98458 | −0.579289258 | 0.0396396 | hypothetical protein |
| CDZ97730 | −0.588026153 | 0.03442525 | WD repeat protein |
| CED83001 | −0.592611944 | 0.0185739 | heat shock protein 60 ame: full = 60 kda chaperonin |
| CED83868 | −0.625916454 | 0.03416979 | succinate dehydrogenase |
| CDZ97731 | −0.643476843 | 0.01109532 | Translocase of outer mitochondrial membrane complex, subunit TOM70/TOM72 |
| CDZ97556 | −0.643931077 | 0.02878294 | hypothetical protein |
| CDZ96847 | −0.645141048 | 0.01932677 | nucleic acid-binding protein |
| CDZ98246 | −0.651973989 | 0.03188712 | Nucleotide excision repair factor NEF2, RAD23 component |
| CED84183 | −0.685890266 | 0.0188681 | carbon-nitrogen hydrolase |
| CDZ97282 | −0.69152768 | 0.02513695 | t-complex protein alpha subunit (tcp-1-alpha) |
| CED82059 | −0.693303008 | 0.04460738 | fk506-binding protein 2 |
| CED85216 | −0.695853389 | 0.04250729 | Aromatic amino acid aminotransferase and related proteins |
| CDZ97115 | −0.702133604 | 0.00468473 | adenylate kinase |
| CDZ96380 | −0.730249947 | 0.0188681 | alpha-aminoadipate reductase lys1p |
| CED82220 | −0.730863676 | 0.02513695 | coatomer protein subunit alpha |
| CED82774 | −0.745400143 | 0.04391875 | 40s ribosomal protein s21 |
| CED83139 | −0.74609568 | 0.00828925 | carbamoyl-phosphate synth |
| CDZ98369 | −0.746457631 | 0.00730273 | Histone acetyltransferase SAGA, TRRAP/TRA1 component, PI-3 kinase superfamily |
| CED82652 | −0.758190818 | 0.04990868 | SAM-dependent RNA methyltransferase, predicted |
| CED83286 | −0.775731741 | 0.01093808 | related to 2-hydroxy-3-oxopropionate reductase |
| CED84878 | −0.779531222 | 0.03376189 | Transcriptional coactivator p100 |
| CDZ97020 | −0.781450259 | 0.02649992 | 60s ribosomal protein I13a |
| CDZ96477 | −0.785705988 | 0.01799604 | ornithine carbamoyltransferase |
| CDZ98039 | −0.788300037 | 0.01799604 | eukaryotic translation initiation factor 5b |
| CED85427 | −0.80342206 | 0.03519539 | aconitate hydratase |
| CED82232 | −0.807033917 | 0.02649992 | rrna 2-o-methyltransferase fibrillarin |
| CED84791 | −0.820220844 | 0.03029551 | t-complex protein beta subunit (tcp-1-beta) |
| CED84188 | −0.835143316 | 0.02856417 | voltage-dependent ion-selective channel |
| CDZ98718 | −0.835331242 | 0.0118088 | coatomer beta subunit |
| CED84980 | −0.840382794 | 0.00778145 | succinate-ligase (adp-forming) |
| CED83765 | −0.85429126 | 0.02649992 | carbamoyl-phosphate synthase |
| CDZ96447 | −0.854577872 | 0.03155268 | related to ste23-metalloprotease involved in a- factor processing |
| CED83665 | −0.85634675 | 0.02060729 | microtubule binding protein |
| CED82100 | −0.857908244 | 0.0396396 | Pyridoxalphosphate-dependent enzyme/predicted threonine synthase |
| CED83464 | −0.868102971 | 0.04122494 | fact complex subunit spt16 |
| CDZ96576 | −0.876356779 | 0.01688491 | sly1 vesicle trafficking sec1-like protein |
| CDZ97524 | −0.883112569 | 0.01168687 | cytoplasm protein |
| CED84444 | −0.893719592 | 0.00229207 | nadh dehydrogenase |
| CED85449 | −0.903173171 | 0.04250729 | 20s proteasome subunit |
| CDZ96270 | −0.918929334 | 0.0020292 | phosphoglycerate kinase |
| CED84681 | −0.929912636 | 0.03631055 | arm repeat-containing protein |
| CDZ97551 | −0.932426135 | 0.03282912 | Ribonuclease III domain |
| CED84960 | −0.938006938 | 0.02264306 | gtp binding protein 4 |
| CED82053 | −0.949623751 | 0.03376189 | peptidyl-prolyl cis-trans isomerase b |
| CDZ96524 | −0.9544128 | 0.01168687 | Translation initiation factor 4F, ribosome/mRNA- bridging subunit (elF-4G) |
| CED83897 | −0.974995763 | 0.02513695 | nucleosome assembly protein |
| CDZ96352 | −0.983332602 | 0.00831414 | eukaryotic polypeptide chain release factor 3 |
| CDZ98525 | −0.992343475 | 0.02215675 | asparagine synthase (glutamine-hydrolyzing) |
| CED84522 | −0.993837467 | 0.04128817 | Splicing factor U2AF, large subunit (RRM superfamily) |
| CED82181 | −1.005480158 | 0.00925982 | nadh-cytochrome b5 reductase |
| CED82591 | −1.029639978 | 0.03787191 | histidine biosynthesis trifunctional-protein |
| CED83723 | −1.037585374 | 0.04122494 | Enoyl-CoA hydratase |
| CDZ97012 | −1.043031541 | 0.01932677 | alcohol |
| CDZ96916 | −1.043355493 | 0.01932677 | imidazoleglycerol phosphate synthase |
| CED84114 | −1.043474748 | 0.03331544 | Thioredoxin-like fold |
| CDZ98026 | −1.044917324 | 0.02651527 | ef-hand |
| CED83652 | −1.05240471 | 0.00905229 | electron-transferring-flavoprotein dehydrogenase |
| CED82273 | −1.056067612 | 0.02914553 | mbf1-domain-containing protein |
| CED82044 | −1.067075615 | 0.02878294 | Transcription factor IIS, N-terminal |
| CED83685 | −1.067321455 | 0.00361302 | heat shock protein 70 |
| CED82614 | −1.073787138 | 0.0020292 | pyruvate carboxylase |
| CDZ96680 | −1.074598877 | 0.01276031 | homoaconitate hydratase |
| CED83939 | −1.078083583 | 0.03043844 | Uncharacterized conserved coiled-coil protein |
| CED83611 | −1.095807523 | 0.0238592 | Alternative splicing factor SRp55/B52/SRp75 (RRM superfamily) |
| CDZ98729 | −1.100728689 | 0.02432014 | eukaryotic translation initiation factor 3 subunit 7 |
| CED83034 | −1.100823166 | 0.00057316 | threonyl-trna synthetase |
| CDZ98402 | −1.103940436 | 0.02748415 | RmIC-like jelly roll fold |
| CED85540 | −1.128024094 | 0.00468473 | 60s ribosomal protein I20 |
| CED83021 | −1.148413349 | 0.04783144 | mRNA splicing factor |
| CDZ97267 | −1.149128196 | 0.00905229 | pre-mrna-processing protein 45 |
| CED82480 | −1.149348994 | 0.04702612 | atp-dependent rrna helicase rrp3 |
| CDZ96746 | −1.154878981 | 0.02549825 | dihydrolipoyllysine-residue acetyltransferase |
| CED83926 | −1.172857388 | 0.04339517 | Acyl-CoA synthetase |
| CDZ98315 | −1.177258586 | 0.02432014 | Ribosomal protein S5 |
| CDZ98765 | −1.177803882 | 0.01508991 | phenylalanyl-trna synthetase subunit beta |
| CED82811 | −1.179117557 | 0.00926928 | wd40 repeat-like protein |
| CDZ98852 | −1.183130469 | 0.01583624 | vacuolar atp synthase subunit d |
| CED83932 | −1.187051314 | 0.03441326 | Phosphatidylserine decarboxylase |
| CDZ98263 | −1.196465477 | 0.02645449 | Vigilin |
| CED84276 | −1.205793039 | 0.02212097 | RNA recognition motif domain |
| CED82490 | −1.21564933 | 0.01253292 | plasma membrane h( )-atpase 1 |
| CED83200 | −1.217961416 | 0.03376189 | RRM motif-containing protein |
| CDZ97483 | −1.218256982 | 0.01389815 | Predicted GTPase-activating protein |
| CED83084 | −1.225292534 | 0.0472272 | F1-ATP synthase assembly protein |
| CDZ97245 | −1.233180145 | 0.00905229 | acetyl-hydrolase |
| CDZ96943 | −1.240309934 | 0.01350904 | Peptidyl-prolyl cis-trans isomerase |
| CDZ97689 | −1.249838431 | 0.02649992 | antiviral helicase |
| CDZ98179 | −1.259964099 | 0.00361302 | Acetyl CoA carboxylase |
| CED84347 | −1.263008364 | 0.01393287 | agc pka protein kinase |
| CED83589 | −1.265861918 | 0.01218618 | ATP-dependent RNA helicase pitchoune |
| CED83658 | −1.266003386 | 0.04316808 | Microtubule-associated protein |
| CED82186 | −1.268044412 | 0.0308089 | Cell-cycle nuclear protein, contains WD-40 repeats |
| CED84113 | −1.27089372 | 0.03126627 | phosphoserine aminotransferase |
| CED84268 | −1.280229433 | 0.02549825 | Vacuolar protein sorting-associated protein |
| CED84243 | −1.280431294 | 0.0018404 | gmp synthase |
| CDZ96172 | −1.287548114 | 0.02941486 | translational regulator gcn20-like abc transporter |
| CED82356 | −1.288291751 | 0.04391875 | GDP-mannose pyrophosphorylase |
| CED84922 | −1.289443888 | 0.00874094 | Acetyl CoA acyltransferase 2 |
| CED84556 | −1.292812239 | 0.01932677 | phosphoketolase |
| CED84796 | −1.295776509 | 0.03376189 | Delta 12 fatty acid desaturase |
| CED83864 | −1.313283154 | 0.02649992 | vacuolar protein 8 |
| CED82178 | −1.320876871 | 0.0118088 | Predicted haloacid-halidohydrolase and related hydrolases |
| CDZ97101 | −1.32734763 | 0.02988308 | peptidyl-prolyl cis-trans isomerase |
| CDZ98445 | −1.335643909 | 0.03297342 | class iii adh enzyme |
| CDZ98528 | −1.335685233 | 0.00905229 | carbamoyl-phosphate synthase |
| CED83469 | −1.348907932 | 0.00905229 | t-complex protein 1 |
| CED85129 | −1.349857903 | 0.03671048 | isocitrate lyase |
| CED83250 | −1.359585261 | 0.02549825 | atpase |
| CED83852 | −1.380864038 | 0.04702612 | 6-phosphogluconolactonase |
| CDZ97571 | −1.389240555 | 0.04290904 | mitochondrial inner membrane protein |
| CED84645 | −1.395950599 | 0.02960533 | t-complex protein 1 subunit delta |
| CED84885 | −1.400831215 | 0.0310697 | adaptor protein complex ap-1 gamma subunit |
| CED84004 | −1.410145406 | 0.03448029 | arm repeat-containing protein |
| CDZ96721 | −1.413395703 | 0.02914553 | rRNA processing protein Rrp5 |
| CDZ97030 | −1.424528912 | 0.00099383 | succinate:fumarate antiporter |
| CED85589 | −1.425848646 | 0.02839911 | Predicted proline-serine-threonine phosphatase- interacting protein (PSTPIP) |
| CED82624 | −1.43305487 | 0.02513695 | phospho-2-dehydro-3-deoxyheptonate aldolase |
| CED84664 | −1.433337418 | 0.00831414 | rna-binding domain-containing protein |
| CED83236 | −1.435153651 | 0.00374945 | epsilon dna polymerase |
| CDZ96438 | −1.436256297 | 0.0182665 | Cullins |
| CED82493 | −1.438548522 | 0.03416979 | asparaginyl-trna synthetase |
| CED83361 | −1.441977148 | 0.00926928 | dihydroxy-acid dehydratase |
| CDZ97327 | −1.448812399 | 0.00828925 | hypothetical protein |
| CDZ96706 | −1.450203913 | 0.01001901 | SNARE protein SED5/Syntaxin 5 |
| CDZ96703 | −1.468072782 | 0.02645449 | centromere microtubule binding protein cbf5 |
| CDZ97922 | −1.483216564 | 0.01093808 | histidyl-trna synthetase |
| CDZ96493 | −1.521855592 | 0.01791703 | Endoplasmic reticulum protein EP58, contains filamin rod domain and KDEL motif |
| CED84450 | −1.533220692 | 0.01109532 | 3-isopropylmalate dehydrogenase |
| CED84039 | −1.537415237 | 0.04054781 | Glycosyl transferase, family 1 |
| CDZ97426 | −1.540107744 | 0.00468097 | eukaryotic translation initiation factor 3 subunit 6 |
| CED83785 | −1.543039464 | 0.04316808 | phosphoglycerate mutase family |
| CED84622 | −1.543931468 | 0.02549825 | chromatin remodelling complex atpase chain |
| CED85268 | −1.553723075 | 0.04128817 | carboxypeptidase s |
| CED84941 | −1.560007044 | 0.01060517 | Predicted hydrolases or acyltransferases (alpha/beta hydrolase superfamily) |
| CDZ96153 | −1.561131772 | 0.04339517 | NADH dehydrogenase subunits 2, 5, and related proteins |
| CDZ97901 | −1.567756257 | 0.03376189 | synaptobrevin-like protein |
| CED85483 | −1.575183227 | 0.00905229 | 40s ribosomal protein s6 |
| CDZ98650 | −1.581282177 | 0.02428145 | Ubiquitin C-terminal hydrolase UCHL1 |
| CED82653 | −1.587084692 | 0.02889048 | PolyC-binding proteins alphaCP-1 and related KH domain proteins |
| CED82987 | −1.587476435 | 0.02432014 | Nucleolar RNA-associated protein (NRAP) |
| CED82302 | −1.589117444 | 0.03376189 | WD40 repeat-containing protein |
| CED83333 | −1.590249823 | 0.0020292 | pyruvate decarboxylase |
| CED84972 | −1.5960981 | 0.01093808 | NADH-dehydrogenase (ubiquinone) |
| CDZ97229 | −1.596189229 | 0.04783144 | RhoGEF GTPase |
| CED82008 | −1.598536492 | 0.01016089 | Ca2-dependent lipid-binding protein CLB1/vesicle protein vp115/Granuphilin A, contains C2 domain |
| CED85558 | −1.602612866 | 0.03649973 | Molecular co-chaperone STI1 |
| CED84694 | −1.608371962 | 0.02031649 | Acyl-CoA synthetase |
| CED85482 | −1.609224816 | 0.02478907 | Vacuolar H-ATPase V1 sector, subunit E |
| CDZ97501 | −1.609942116 | 0.04201509 | p-loop containing nucleoside triphosphate hydrolase protein |
| CED82887 | −1.614733759 | 0.02490967 | Spliceosome subunit |
| CED82373 | −1.617336057 | 0.01331504 | Microtubule-binding protein involved in cell cycle control |
| CED85608 | −1.627956219 | 0.01688491 | Karyopherin (importin) beta 3 |
| CDZ96772 | −1.633635875 | 0.0215727 | dna-dependent rna polymerase ii second largest subunit |
| CED82875 | −1.636243466 | 0.02878294 | p-loop containing nucleoside triphosphate hydrolase protein |
| CDZ96709 | −1.643701581 | 0.01276031 | coatomer subunit gamma |
| CED82718 | −1.653749043 | 0.02914553 | Dehydrogenase kinase |
| CDZ98379 | −1.654855765 | 0.03376189 | mitochondrial pyruvate dehydrogenase e1 component beta subunit |
| CDZ98664 | −1.664987389 | 0.00828925 | glycoside hydrolase family 13 protein |
| CDZ97227 | −1.676730007 | 0.01799604 | nad-specific glutamate dehydrogenase |
| CED84865 | −1.693137102 | 0.02925005 | mitochondrial 50s ribosomal protein I3 |
| CDZ97357 | −1.69780688 | 0.01583624 | Ran GTPase-activating protein |
| CDZ97274 | −1.703158642 | 0.04128817 | fkbp-type peptidyl-prolyl cis-trans isomerase |
| CED85510 | −1.704437647 | 0.03376189 | hypothetical protein |
| CDZ98568 | −1.707428296 | 0.01799604 | 60s ribosomal protein I19 |
| CDZ97211 | −1.708555431 | 0.0195227 | Small Nuclear ribonucleoprotein splicing factor |
| CED84798 | −1.717516704 | 0.01276031 | Mannosyltransferase |
| CDZ98483 | −1.722444299 | 0.02914553 | dutp pyrophosphatase |
| CED82387 | −1.7250923 | 0.01109532 | GST, gst |
| CED84167 | −1.738514851 | 0.01583624 | glutamate-trna ligase |
| CDZ98702 | −1.740168961 | 0.01799604 | mov34-domain-containing protein |
| CED82610 | −1.741145108 | 0.01389815 | mitochondrial nuclease |
| CDZ97968 | −1.747047833 | 0.04735782 | 1,4-benzoquinone reductase-like |
| CED85256 | −1.763029564 | 0.03716115 | thiamine biosynthetic bifunctional enzyme |
| CED83533 | −1.763463174 | 0.02889048 | Protein of unknown function DUF3602 |
| CDZ96990 | −1.765718629 | 0.01508991 | upf0041-domain-containing protein |
| CED83331 | −1.766842575 | 0.01575712 | 60s ribosomal protein I11 |
| CED83746 | −1.777546216 | 0.03227504 | hypothetical protein |
| CDZ97319 | −1.785289098 | 0.02212097 | Serine/threonine protein phosphatase 2A, regulatory subunit |
| CDZ98624 | −1.788285059 | 0.03416979 | argininosuccinate lyase |
| CED85213 | −1.790475985 | 0.00905229 | elongation factor 1 beta delta chain |
| CED83192 | −1.803912495 | 0.02988308 | bar-domain-containing protein |
| CED84181 | −1.815242741 | 0.01154058 | uridylate kinase |
| CDZ96394 | −1.816977653 | 0.04455459 | phosphatidylethanolamine n-methyltransferase |
| CDZ98091 | −1.818027401 | 0.01389815 | stomatin family protein |
| CED83762 | −1.820314128 | 0.03448029 | ubiquitin-conjugating enzyme |
| CED85505 | −1.834634216 | 0.01109532 | glycosyltransferase family 2 protein |
| CED84800 | −1.838524109 | 0.04128817 | hypothetical protein |
| CED84682 | −1.839194002 | 0.03631055 | Uncharacterized conserved protein |
| CED83350 | −1.852972209 | 0.0163988 | signal recognition particle protein |
| CED85299 | −1.854408376 | 0.0110087 | B-cell receptor-associated protein and related proteins |
| CED82175 | −1.859771924 | 0.01109532 | RNA-binding S4 domain |
| CDZ97972 | −1.872513626 | 0.03376189 | Drebrins and related actin binding proteins |
| CED82401 | −1.880909466 | 0.03376189 | small gtpase-binding protein |
| CDZ98059 | −1.883204159 | 0.02914553 | gtp cyclohydrolase i |
| CED84722 | −1.892048206 | 0.02649992 | ps16 protein |
| CED85186 | −1.895077938 | 0.03376189 | eukaryotic translation initiation factor 3 subunit 6 |
| CED84608 | −1.909008315 | 0.00730273 | hypothetical protein |
| CDZ97780 | −1.93325344 | 0.01276031 | hypothetical protein |
| CDZ96174 | −1.939004053 | 0.04792978 | Predicted hydrolase related to dienelactone hydrolase |
| CED84225 | −1.953742748 | 0.02748415 | Nuclear localization sequence binding protein |
| CED83904 | −1.954139811 | 0.02914553 | swi snf complex protein |
| CDZ96983 | −1.969755024 | 0.01462763 | hypothetical protein |
| CED84490 | −1.981429905 | 0.01164179 | gtp-binding protein ypt1 |
| CDZ96924 | −1.982246388 | 0.0423797 | ATPase, F0 complex, subunit H |
| CED82203 | −1.986159712 | 0.02649992 | metal resistance protein ycf1 |
| CED83393 | −2.006367081 | 0.02513695 | Outer membrane protein, MIM1/TOM13, mitochondrial |
| CDZ96807 | −2.019757778 | 0.04399915 | ubiquitin-protein ligase molybdopterin-converting factor |
| CDZ97740 | −2.025807045 | 0.01799604 | gtp-binding protein |
| CDZ96605 | −2.027729754 | 0.00229207 | Predicted mitochondrial carrier protein |
| CDZ97834 | −2.031082152 | 0.0185739 | rna-binding domain-containing protein |
| CED82245 | −2.038685375 | 0.03019913 | 28 kda golgi snare protein |
| CED83853 | −2.040204572 | 0.0070558 | dead-domain-containing protein |
| CED84219 | −2.042496249 | 0.01505725 | -trehalose-phosphate synthase (udp-forming) |
| CDZ96300 | −2.058119 | 0.02914553 | ran protein binding protein |
| CED82557 | −2.061553008 | 0.02748415 | pkinase-domain-containing protein |
| CED83678 | −2.063129603 | 0.03376189 | hypothetical protein |
| CDZ97373 | −2.067413471 | 0.0118088 | Ribosome recycling factor domain |
| CED82649 | −2.069249285 | 0.01109532 | phosphatase |
| CED85233 | −2.082511905 | 0.03019913 | 20s proteasome subunit |
| CDZ96426 | −2.089091053 | 0.02549825 | Nucleic acid-binding, OB-fold |
| CED84077 | −2.089257714 | 0.03043844 | atp-dependent rna helicase dbp5 |
| CDZ97849 | −2.094972329 | 0.01408931 | mRNA export protein (contains WD40 repeats) |
| CDZ97098 | −2.104764907 | 0.02264306 | Protein phosphatase 2A regulatory subunit A and related proteins |
| CDZ96939 | −2.109542723 | 0.01649499 | glutaminyl-trna synthetase |
| CED85158 | −2.11839784 | 0.02914553 | Prolactin regulatory element-binding protein/Protein transport protein SEC12p |
| CDZ97144 | −2.124633505 | 0.01508991 | Ribosome Assembly protein |
| CED82147 | −2.141163817 | 0.02107155 | C4-type Zn-finger protein |
| CED82043 | −2.145468246 | 0.00521227 | Exosomal 3′-5′ exoribonuclease complex subunit Rrp40 |
| CED84757 | −2.14801929 | 0.02513695 | Transcription regulator HTH, APSES-type DNA-binding domain |
| CED82578 | −2.169428299 | 0.01222391 | hypothetical protein |
| CED84478 | −2.18760405 | 0.02800499 | RIB7, arfC |
| CED83083 | −2.18959859 | 0.04028758 | 60s ribosomal protein I12 |
| CDZ98457 | −2.202848188 | 0.02878294 | guanylate kinase |
| CED85582 | −2.214308627 | 0.02344303 | Predicted membrane protein |
| CED82465 | −2.237750259 | 0.04122494 | glycerol-3-phosphate o-acyltransferase |
| CED85428 | −2.244873713 | 0.02203629 | Cactin |
| CED82211 | −2.259799008 | 0.01218618 | translation initiation factor eif3 subunit |
| CDZ97947 | −2.280864979 | 0.02064593 | hypothetical protein |
| CDZ96867 | −2.283479884 | 0.04861292 | Biotin holocarboxylase synthetase/biotin-protein ligase |
| CDZ97011 | −2.299863258 | 0.00164427 | 60s ribosomal protein I23 |
| CDZ96733 | −2.311515815 | 0.04054781 | Inositol monophosphatase |
| CED84213 | −2.315052185 | 0.00816695 | ras-domain-containing protein |
| CED83529 | −2.316671565 | 0.00208188 | Maltase glucoamylase and related hydrolases, glycosyl hydrolase family 31 |
| CED82724 | −2.319660064 | 0.02026839 | Ribosomal protein S24/S35, mitochondrial, conserved domain |
| CED83468 | −2.36448386 | 0.04092267 | Peptide methionine sulfoxide reductase |
| CDZ96813 | −2.365957431 | 0.0188681 | hypothetical protein |
| CDZ97212 | −2.366141588 | 0.02365796 | mitochondrial carrier |
| CED83014 | −2.380596735 | 0.01583624 | nad-dependent formate dehydrogenase |
| CDZ98614 | −2.387150324 | 0.0238592 | Molecular chaperone (DnaJ superfamily) |
| CDZ97075 | −2.394628831 | 0.03019913 | hypothetical protein |
| CED82399 | −2.402453576 | 0.02264306 | hypothetical protein |
| CDZ98618 | −2.404051464 | 0.02649992 | Immunoglobulin-like fold |
| CED84473 | −2.413270708 | 0.00905229 | Translational repressor Pumilio/PUF3 and related RNA-binding proteins (Puf superfamily) |
| CED82408 | −2.41927523 | 0.0195415 | urease accessory protein |
| CED84497 | −2.49808343 | 0.02856417 | modular protein with glycoside hydrolase family 13 and glycosyltransferase family 5 domains |
| CDZ98509 | −2.522541548 | 0.04122494 | orotidine-5-phosphate decarboxylase |
| CED85305 | −2.529111979 | 0.03376189 | Phosphoprotein/predicted coiled-coil protein |
| CED83414 | −2.530683213 | 0.02889048 | nucleosome remodeling subunit caf1 nurf55 msi1 |
| CDZ97207 | −2.557565145 | 0.04227026 | Zinc finger, RING/FYVE/PHD-type |
| CED83149 | −2.582726156 | 0.02212097 | Prefoldin subunit 6, KE2 family |
| CED83584 | −2.597234359 | 0.0238592 | hypothetical protein |
| CDZ98446 | −2.624539223 | 0.04976639 | thioredoxin h |
| CED83860 | −2.651386968 | 0.03649973 | adf-like domain-containing protein |
| CDZ98236 | −2.710676815 | 0.00361302 | Alcohol dehydrogenase, class V |
| CED84022 | −2.725408421 | 0.0296611 | 60s ribosomal protein I13 |
| CDZ97924 | −2.727453323 | 0.03021911 | hypothetical protein |
| CED83962 | −2.754374803 | 0.02513695 | glycoside hydrolase family 3 protein |
| CED84144 | −2.826532427 | 0.02060729 | Uncharacterized conserved protein |
| CED82710 | −2.848141147 | 0.02649992 | hypothetical protein |
| spiRT_peptides | −2.91938355 | 0.02549825 | |
| CED83656 | −2.929874549 | 0.02889048 | Delta 9 fatty acid desaturase |
| CDZ97129 | −2.950421262 | 0.01445079 | Predicted regulator of rRNA gene transcription (MYB-binding protein) |
| CDZ96235 | −2.965311288 | 0.03160153 | Regulator of ribosome synthesis |
| CDZ98212 | −2.984446161 | 0.00164427 | WD40 repeat-containing protein |
| CDZ96176 | −3.011422884 | 0.02221332 | hexose transport-related protein |
| CED83954 | −3.013895262 | 0.01541525 | hypothetical protein |
| CED82845 | −3.017036891 | 0.01649499 | protein-histidine kinase |
| CED83160 | −3.02768041 | 0.02344303 | dna-directed rna polymerase ii subunit i |
| CED85402 | −3.073843435 | 0.01168687 | inositol-3-phosphate synthase |
| CDZ98485 | −3.089642931 | 0.03416979 | protein transport protein sec22 |
| CED82033 | −3.191081754 | 0.04783144 | hypothetical protein |
| CDZ98496 | −3.283958645 | 0.01943629 | hypothetical protein |
| CED82966 | −3.393186712 | 0.0188681 | Taurine catabolism dioxygenase TauD/TfdA |
| CED82706 | −4.028618494 | 0.04727779 | ATPase inhibitor, IATP, mitochondria |
| CED84836 | −4.452914746 | 0.01439857 | hypothetical protein |
| CDZ96436 | −4.833255417 | 0.00066477 | alcohol |

TABLE 19

| Protein | log2FC | adj. pvalue | Protein name |
|---|---|---|---|
| CED82765 | 4.231508 | 0.0223966 | Quinonprotein alcohol dehydrogenase-like superfamily |
| CDZ96711 | 3.818548 | 0.0001057 | short-chain dehydrogenase |
| CDZ96246 | 3.561085 | 0.1088072 | NADH-ubiquinone oxidoreductase, 21 kDa subunit, N- terminal |
| CDZ96507 | 3.488467 | 0.0608974 | RmlC-like jelly roll fold |
| CDZ98452 | 3.28816 | 0.0026732 | hypothetical protein |
| CED82658 | 3.161995 | 0.0056835 | Deoxyribodipyrimidine photolyase/cryptochrome |
| CDZ96283 | 3.050237 | 0.0858571 | hypothetical protein |
| CDZ97053 | 2.989734 | 0.0771834 | 40s ribosomal protein s15 |
| CED82874 | 2.544518 | 0.0751553 | gdp-I-fucose synthetase |
| CED85319 | 2.31452 | 0.0859067 | cysteine proteinase |
| CDZ96948 | 2.310015 | 0.1080156 | Protein phosphatase 1, regulatory subunit, and related proteins |
| CED83221 | 2.288329 | 0.0494045 | acn9-domain-containing protein |
| CED85064 | 2.19761 | 0.0871441 | actin depolymerizing factor |
| CDZ98400 | 2.176685 | 0.0222833 | proline iminopeptidase |
| CDZ97081 | 2.10351 | 0.0433728 | cyclophilin family peptidyl-prolyl cis-trans isomerase cyp2 |
| CED83979 | 2.095009 | 0.042078 | Flavonol reductase/cinnamoyl-CoA reductase |
| CDZ98539 | 2.094727 | 0.0462864 | SNARE protein Syntaxin 1 and related proteins |
| CED82343 | 2.043645 | 0.1001054 | serine threonine-protein phosphatase pp1 |
| CDZ98291 | 1.972938 | 0.0126954 | carboxypeptidase s |
| CED85501 | 1.949842 | 0.0293803 | nucleosome assembly protein |
| CDZ96399 | 1.929302 | 0.0342256 | Manganese/iron superoxide dismutase, C-terminal |
| CDZ98540 | 1.883129 | 0.0146614 | glycine hydroxymethyltransferase |
| CDZ96534 | 1.875855 | 0.0313407 | dihydroorotase |
| CDZ97688 | 1.871465 | 0.0962559 | glutaredoxin-related protein |
| CED84517 | 1.856004 | 0.0514783 | Protein required for normal rRNA processing |
| CDZ98438 | 1.840714 | 0.1080156 | hit family protein 1 |
| CDZ97677 | 1.834903 | 0.0147147 | Mitochondrial F1F0-ATP synthase, subunit OSCP/ATP5 |
| CED82582 | 1.820626 | 0.067386 | 20s proteasome subunit beta 3 |
| CED82590 | 1.788278 | 0.0137624 | Thioredoxin-like protein |
| CDZ97777 | 1.77895 | 0.1232547 | hypothetical protein |
| CED82380 | 1.776832 | 0.17568 | Predicted metal-binding protein |
| CDZ97534 | 1.701239 | 0.0190789 | Predicted dehydrogenase |
| CDZ98470 | 1.682504 | 0.1568805 | short-chain dehydrogenase |
| CED85564 | 1.678797 | 0.0044979 | Carbon-nitrogen hydrolase |
| CED82723 | 1.676945 | 0.067386 | Armadillo/beta-catenin-like repeat-containing protein |
| CED82970 | 1.676565 | 0.0233499 | 40s ribosomal protein s22 |
| CDZ97699 | 1.63503 | 0.1706431 | hypothetical protein |
| CDZ96647 | 1.60157 | 0.1214901 | TPMT family |
| CED84186 | 1.583796 | 0.0284367 | dna-directed rna polymerases and iii kda polypeptide |
| CED82344 | 1.533073 | 0.0512013 | Glycosyl transferase, family 8-glycogenin |
| CDZ96748 | 1.510136 | 0.1080156 | short-chain dehydrogenase |
| CDZ97010 | 1.506639 | 0.1232547 | Coproporphyrinogen III oxidase CPO/HEM13 |
| CDZ96544 | 1.475695 | 0.1378419 | 40s ribosomal protein s19 |
| CED83819 | 1.457086 | 0.17568 | adp-ribosylation factor |
| CED85610 | 1.456115 | 0.0460038 | Salt-sensitive 3′-phosphoadenosine-5′-phosphatase HAL2/SAL1 |
| CED83449 | 1.448604 | 0.0419656 | phytoene-beta carotene synthase |
| CED83814 | 1.431916 | 0.1150853 | acyl-oxidase |
| CED83513 | 1.427821 | 0.0026732 | phytoene dehydrogenase |
| CED84484 | 1.41827 | 0.1309145 | Peripheral-type benzodiazepine receptor and related proteins |
| CED85105 | 1.417928 | 0.0552523 | cytochrome-c oxidase chain vi precursor |
| CDZ97542 | 1.417535 | 0.0560071 | 60s acidic ribosomal protein p0 |
| CED84758 | 1.387994 | 0.0175187 | ubc-like protein |
| CED82388 | 1.380273 | 0.0126954 | Cytochrome b5 |
| CDZ97342 | 1.320494 | 0.1706431 | Voltage-gated shaker-like K channel, subunit beta/KCNAB |
| CED83339 | 1.297949 | 0.1706431 | Phox/Bem1p |
| CED83687 | 1.280689 | 0.0561539 | mitochondrial protein |
| CED83477 | 1.267639 | 0.1001054 | Uncharacterized mRNA-associated protein RAP55 |
| CED82722 | 1.25782 | 0.0126954 | aspartyl aminopeptidase |
| CED85079 | 1.246104 | 0.0982665 | Cytokinin riboside 5′-monophosphate phosphoribohydrolase LOG |
| CDZ97430 | 1.235358 | 0.1175347 | phosphomevalonate kinase |
| CDZ97936 | 1.205686 | 0.1704925 | Histone deacetylase complex, SIN3 component |
| CDZ98639 | 1.203896 | 0.0503176 | Aromatic amino acid aminotransferase and related proteins |
| CED82942 | 1.194279 | 0.0876816 | G protein beta subunit-like protein |
| CED83940 | 1.151573 | 0.1305788 | astaxanthin synthase |
| CDZ96405 | 1.130725 | 0.1271256 | phospho protein phosphatase ppz |
| CED85454 | 1.126577 | 0.1740412 | Putative metallopeptidase |
| CED82502 | 1.126374 | 0.033781 | nascent polypeptide-associated complex subunit beta |
| CDZ96583 | 1.116411 | 0.0050139 | ferrochelatase |
| CDZ96773 | 1.090504 | 0.0297939 | Translocase of outer mitochondrial membrane complex, subunit TOM20 |
| CDZ96425 | 1.0814 | 0.0514783 | catalase 1 |
| CED83372 | 1.071293 | 0.106338 | ATPase, F0 complex, subunit J |
| CED85327 | 1.026518 | 0.0982665 | carboxypeptidase s |
| CDZ97990 | 1.014943 | 0.0763925 | cwf21-domain-containing protein |
| CED84048 | 1.014714 | 0.1715042 | dsRNA-activated protein kinase inhibitor P58, contains TPR and DnaJ domains |
| CDZ96728 | 0.981699 | 0.0962559 | Voltage-gated shaker-like K channel, subunit beta/KCNAB |
| CDZ97775 | 0.956662 | 0.147114 | serine carboxypeptidase |
| CDZ96256 | 0.936691 | 0.0962559 | EF-hand domain pair |
| CED82360 | 0.933276 | 0.1042988 | hypothetical protein |
| CDZ98787 | 0.909721 | 0.1042988 | Predicted transporter (major facilitator superfamily) |
| CED84688 | 0.906571 | 0.1378419 | Aldo/keto reductase family proteins |
| CED83668 | 0.886642 | 0.0994446 | 26S proteasome regulatory complex, subunit RPN12/PSMD8 |
| CDZ98576 | 0.88097 | 0.0947646 | isocitrate dehydrogenase |
| CED84981 | 0.870208 | 0.1309145 | Iron/ascorbate family oxidoreductases |
| CED82222 | 0.859981 | 0.026263 | udp-glucose 4-epimerase |
| CED83351 | 0.848954 | 0.0037331 | abc transporter |
| CED83536 | 0.799797 | 0.0152746 | N-terminal acetyltransferase |
| CED85376 | 0.797626 | 0.0376685 | glyceraldehyde-3-phosphate dehydrogenase |
| CED83362 | 0.79424 | 0.1348598 | enolase |
| CDZ97741 | 0.772249 | 0.0126954 | prtase-like protein |
| CDZ98574 | 0.770517 | 0.1709508 | Protein of unknown function DUF4336 |
| CDZ96706 | 0.754757 | 0.1158743 | SNARE protein SED5/Syntaxin 5 |
| CDZ97607 | 0.735133 | 0.0876816 | er-associated protein catabolism-related protein |
| CED85341 | 0.732743 | 0.1007998 | Armadillo-type fold |
| CED84995 | 0.728884 | 0.0270372 | nadh-ubiquinone oxidoreductase 304 kda subunit precursor |
| CED82933 | 0.727752 | 0.0137624 | cndp dipeptidase |
| CED83153 | 0.722774 | 0.0050139 | aaa atpase |
| CDZ98090 | 0.718506 | 0.1709508 | adenylosuccinate lyase |
| CED83950 | 0.717521 | 0.1437425 | Beta-tubulin folding cofactor A |
| CDZ96168 | 0.702449 | 0.0486467 | 26s proteasome subunit p45 |
| CDZ96582 | 0.698475 | 0.0126954 | 26s proteasome subunit p45 |
| CED82968 | 0.695251 | 0.0886062 | Protein of unknown function DUF4449 |
| CED82394 | 0.677589 | 0.1229557 | protein transporter sec23 |
| CDZ96849 | 0.673448 | 0.0126954 | udp-glucuronic acid decarboxylase uxs1p |
| CDZ97626 | 0.588913 | 0.1740412 | FOG: RCC1 domain |
| CDZ98437 | 0.553298 | 0.1295739 | 26s proteasome regulatory atpase rpt6 |
| CED85458 | 0.552718 | 0.147114 | methylenetetrahydrofolate dehydrogenase (nad) |
| CED85255 | 0.518057 | 0.1605827 | serine threonine-protein kinase |
| CDZ97299 | 0.50117 | 0.1214901 | prolyl oligopeptidase |
| CDZ98246 | 0.498631 | 0.1224769 | Nucleotide excision repair factor NEF2, RAD23 component |
| CED85160 | 0.482933 | 0.0920586 | prolyl-trna synthetase |
| CDZ97055 | 0.461666 | 0.1715042 | Predicted hydrolase related to dienelactone hydrolase |
| CED84052 | 0.426502 | 0.1295739 | snare protein ykt6 |
| CED83796 | 0.420471 | 0.0763925 | clathrin heavy chain |
| CDZ96488 | 0.315547 | 0.0876816 | sulfite reductase subunit beta |
| CDZ97115 | −0.28538 | 0.1309145 | adenylate kinase |
| CED84486 | −0.28928 | 0.067386 | Protein disulfide isomerase (prolyl 4-hydroxylase beta subunit) |
| CDZ96784 | −0.31858 | 0.1740412 | nop domain-containing protein |
| CED82089 | −0.32425 | 0.1740412 | fthfs-domain-containing protein |
| CED85467 | −0.32433 | 0.1194556 | pre-mrna-processing-splicing factor |
| CDZ98125 | −0.38528 | 0.1295739 | alanine-trna ligase |
| CDZ97113 | −0.40583 | 0.0039799 | elongation factor 3 |
| CDZ96742 | −0.44015 | 0.1362808 | ck1 ck1 ck1-d protein kinase |
| CDZ97730 | −0.44278 | 0.1363624 | WD repeat protein |
| CED84752 | −0.45235 | 0.147114 | acetylglutamate kinase arg6 |
| CED83281 | −0.4789 | 0.0993953 | succinate-semialdehyde dehydrogenase |
| CED83652 | −0.4896 | 0.1437425 | electron-transferring-flavoprotein dehydrogenase |
| CED83072 | −0.49434 | 0.1232547 | probable eukaryotic translation initiation factor eif-3 |
| CED83685 | −0.50318 | 0.0771834 | heat shock protein 70 |
| CED82409 | −0.50357 | 0.1214901 | Nuclear pore complex component (sc Seh1) |
| CED83447 | −0.50974 | 0.0871441 | 40s ribosomal protein s9 |
| CED85459 | −0.51688 | 0.1305788 | mrna export factor elf1 |
| CED84188 | −0.51802 | 0.1780931 | voltage-dependent ion-selective channel |
| CDZ96380 | −0.53846 | 0.0840022 | alpha-aminoadipate reductase lys1p |
| CDZ98458 | −0.55225 | 0.0833734 | hypothetical protein |
| CDZ97062 | −0.55468 | 0.0486384 | heat shock protein |
| CED85365 | −0.56215 | 0.1221611 | dna-directed rna polymerases i and iii 40 kda polypeptide |
| CDZ98494 | −0.56276 | 0.0678771 | eukaryotic translation initiation factor 3 subunit 8 |
| CDZ98088 | −0.57308 | 0.0771834 | p-loop containing nucleoside triphosphate hydrolase protein |
| CED84980 | −0.58057 | 0.0366021 | succinate-ligase (adp-forming) |
| CED83665 | −0.58523 | 0.1082151 | microtubule binding protein |
| CDZ98525 | −0.58767 | 0.1671066 | asparagine synthase (glutamine-hydrolyzing) |
| CED82622 | −0.59254 | 0.0561539 | aspartate aminotransferase |
| CDZ97426 | −0.59941 | 0.1442533 | eukaryotic translation initiation factor 3 subunit 6 |
| CED83056 | −0.6003 | 0.0453207 | nadh-ubiquinone oxidoreductase |
| CED82811 | −0.60717 | 0.1150853 | wd40 repeat-like protein |
| CED85357 | −0.61544 | 0.0453661 | glycosyltransferase family 35 protein |
| CED82178 | −0.61759 | 0.175572 | Predicted haloacid-halidohydrolase and related hydrolases |
| CED82614 | −0.61903 | 0.0270372 | pyruvate carboxylase |
| CDZ97815 | −0.62037 | 0.1214901 | arginyl-trna synthetase |
| CED83286 | −0.62139 | 0.0336204 | related to 2-hydroxy-3-oxopropionate reductase |
| CDZ97020 | −0.62766 | 0.0898908 | 60s ribosomal protein I13a |
| CDZ97800 | −0.6307 | 0.0876816 | Uncharacterized protein CLU1/cluA |
| CED82608 | −0.63681 | 0.1621596 | 20s proteasome subunit |
| CED84183 | −0.63746 | 0.0404174 | carbon-nitrogen hydrolase |
| CED83611 | −0.6446 | 0.1739511 | Alternative splicing factor SRp55/B52/SRp75 (RRM superfamily) |
| CDZ98039 | −0.64953 | 0.0560934 | eukaryotic translation initiation factor 5b |
| CED82220 | −0.65032 | 0.0636023 | coatomer protein subunit alpha |
| CDZ98017 | −0.67442 | 0.1739511 | oligosaccharyl transferase stt3 subunit |
| CDZ98852 | −0.67733 | 0.1359709 | vacuolar atp synthase subunit d |
| CED84347 | −0.68098 | 0.1436534 | agc pka protein kinase |
| CDZ98026 | −0.6936 | 0.147114 | ef-hand |
| CDZ96254 | −0.69919 | 0.1715042 | phospholipid-translocating atpase |
| CED82282 | −0.69928 | 0.0126954 | polyadenylate-binding protein |
| CED84522 | −0.70055 | 0.180053 | Splicing factor U2AF, large subunit (RRM superfamily) |
| CED85187 | −0.70282 | 0.1080156 | Cullins |
| CDZ96402 | −0.70365 | 0.067386 | hypothetical protein |
| CDZ97267 | −0.70539 | 0.0682198 | pre-mrna-processing protein 45 |
| CDZ97731 | −0.71288 | 0.008825 | Translocase of outer mitochondrial membrane complex, subunit TOM70/TOM72 |
| CED84243 | −0.71406 | 0.023715 | gmp synthase |
| CED84922 | −0.71629 | 0.0858571 | Acetyl CoA acyltransferase 2 |
| CDZ97556 | −0.71738 | 0.033781 | hypothetical protein |
| CED84878 | −0.72579 | 0.0771834 | Transcriptional coactivator p100 |
| CED82908 | −0.728 | 0.0514783 | Metallopeptidase |
| CED84394 | −0.73313 | 0.10398 | Protein of unknown function DUF938 |
| CED82377 | −0.73346 | 0.0695725 | phosphoglucomutase |
| CED83089 | −0.73572 | 0.1296054 | NADH-cytochrome b-5 reductase |
| CED82587 | −0.74374 | 0.1709508 | hypothetical protein |
| CED83181 | −0.75247 | 0.0851343 | eukaryotic translation initiation factor 2 subunit alpha |
| CED83463 | −0.75304 | 0.014415 | atp-dependent rna helicase dhx8 |
| CED82100 | −0.75699 | 0.1007998 | Pyridoxalphosphate-dependent enzyme/predicted threonine synthase |
| CED82249 | −0.76428 | 0.0614175 | Lysophospholipase |
| CED84107 | −0.77092 | 0.0561539 | dead-domain-containing protein |
| CED83380 | −0.78096 | 0.1080156 | Oligosaccharyltransferase, alpha subunit (ribophorin I) |
| CED84444 | −0.78774 | 0.0050139 | nadh dehydrogenase |
| CDZ97088 | −0.78881 | 0.0611706 | gtp binding protein |
| CED84659 | −0.79415 | 0.0830572 | pentafunctional protein |
| CDZ98670 | −0.79644 | 0.1359709 | glycoside hydrolase family 13 protein |
| CED85615 | −0.79864 | 0.1738004 | HMG box-containing protein |
| CED85600 | −0.81236 | 0.0851343 | Acyl-CoA synthetase |
| CED83469 | −0.81611 | 0.0692198 | t-complex protein 1 |
| CED84268 | −0.81772 | 0.1568805 | Vacuolar protein sorting-associated protein |
| CED83034 | −0.82036 | 0.0026732 | threonyl-trna synthetase |
| CED83688 | −0.82625 | 0.0075757 | acetate--ligase |
| CED82774 | −0.82864 | 0.056208 | 40s ribosomal protein s21 |
| CED85021 | −0.82999 | 0.014415 | pkinase-domain-containing protein |
| CDZ96352 | −0.83139 | 0.016916 | eukaryotic polypeptide chain release factor 3 |
| CDZ98212 | −0.84074 | 0.1739511 | WD40 repeat-containing protein |
| CED82307 | −0.84346 | 0.126792 | mevalonate kinase |
| CED84984 | −0.84578 | 0.1150853 | developmentally regulated gtp-binding protein |
| CED82186 | −0.85369 | 0.1657161 | Cell-cycle nuclear protein, contains WD-40 repeats |
| CED82250 | −0.8599 | 0.095967 | coatomer protein |
| CDZ97948 | −0.86811 | 0.1089294 | coatomer subunit delta |
| CDZ98402 | −0.87314 | 0.0979159 | RmlC-like jelly roll fold |
| CDZ98765 | −0.89458 | 0.0608974 | phenylalanyl-trna synthetase subunit beta |
| CED84877 | −0.90761 | 0.1733975 | Nuclear pore complex component (sc Nup85) |
| CED84055 | −0.91098 | 0.0026732 | Fatty acid synthase complex subunit beta |
| CED84731 | −0.91787 | 0.0982665 | cAMP-dependent protein kinase types I and II, regulatory subunit |
| CED84608 | −0.92458 | 0.1043385 | hypothetical protein |
| CDZ97164 | −0.94756 | 0.0957369 | Phosphomethylpyrimidine kinase |
| CDZ96172 | −0.95622 | 0.1229731 | translational regulator gcn20-like abc transporter |
| CDZ98173 | −0.95779 | 0.0692198 | methionyl-trna synthetase |
| CED85540 | −0.96249 | 0.0109769 | 60s ribosomal protein I20 |
| CDZ96447 | −0.96398 | 0.0374233 | related to ste23-metalloprotease involved in a- factor processing |
| CED85618 | −0.96416 | 0.1173322 | glutamate dehydrogenase (nadp ) |
| CDZ97095 | −0.96481 | 0.0222833 | Uncharacterized enzymes related to aldose 1- epimerase |
| CDZ97844 | −0.9676 | 0.0871441 | glycerol-3-phosphate dehydrogenase |
| CDZ96270 | −0.96864 | 0.0026273 | phosphoglycerate kinase |
| CDZ96943 | −0.96912 | 0.0495848 | Peptidyl-prolyl cis-trans isomerase |
| CED84004 | −0.97124 | 0.1715042 | arm repeat-containing protein |
| CED84854 | −0.97158 | 0.0062306 | heat shock protein |
| CED82490 | −0.97251 | 0.042078 | plasma membrane h( )-atpase 1 |
| CED84115 | −0.97573 | 0.0771834 | 2-hydroxyacyl-lyase |
| CDZ96746 | −0.97629 | 0.0751553 | dihydrolipoyllysine-residue acetyltransferase |
| CED84246 | −0.98073 | 0.0175187 | citrate synthase |
| CED84490 | −0.98102 | 0.1512838 | gtp-binding protein ypt1 |
| CED83658 | −0.98434 | 0.1491807 | Microtubule-associated protein |
| CDZ97241 | −0.98541 | 0.0167343 | mitochondrial nad-homo-isocitrate dehydrogenase |
| CED83932 | −0.98644 | 0.1080156 | Phosphatidylserine decarboxylase |
| CED85039 | −0.99117 | 0.067386 | C-24 methyl transferase |
| CDZ98702 | −1.00312 | 0.147114 | mov34-domain-containing protein |
| CED82008 | −1.00466 | 0.0699152 | Ca2-dependent lipid-binding protein CLB1/vesicle protein vp115 |
| CED84681 | −1.01627 | 0.0503176 | arm repeat-containing protein |
| CED84622 | −1.01707 | 0.1438478 | chromatin remodelling complex atpase chain |
| CDZ97595 | −1.01773 | 0.133613 | glycoside hydrolase family 32 protein |
| CDZ96916 | −1.018 | 0.033781 | imidazoleglycerol phosphate synthase |
| CDZ97922 | −1.02091 | 0.0560934 | histidyl-trna synthetase |
| CED82181 | −1.02603 | 0.010389 | nadh-cytochrome b5 reductase |
| CED85375 | −1.03089 | 0.1718815 | 3-deoxy-7-phosphoheptulonate synthase |
| CDZ98718 | −1.03454 | 0.0061469 | coatomer beta subunit |
| CDZ98706 | −1.03631 | 0.0420255 | Phosphatidylinositol transfer protein SEC14 and related proteins |
| CED83084 | −1.03953 | 0.1289963 | F1-ATP synthase assembly protein |
| CDZ97544 | −1.04039 | 0.1512838 | 40s ribosomal protein s20 |
| CED85505 | −1.04548 | 0.1017034 | glycosyltransferase family 2 protein |
| CED85306 | −1.05206 | 0.0561539 | Allantoicase |
| CED83834 | −1.0581 | 0.1080156 | mfs monosaccharide transporter |
| CED82858 | −1.06144 | 0.1232547 | hexokinase |
| CED85483 | −1.06172 | 0.0499431 | 40s ribosomal protein s6 |
| CED83589 | −1.06427 | 0.033781 | ATP-dependent RNA helicase pitchoune |
| CDZ97357 | −1.06568 | 0.1080156 | Ran GTPase-activating protein |
| CED84737 | −1.07129 | 0.0146614 | nad binding dehydrogenase |
| CED83001 | −1.07233 | 0.0026732 | heat shock protein 60 ame: full = 60 kda chaperonin |
| CED83179 | −1.07579 | 0.0137624 | Perilipin |
| CDZ97007 | −1.08006 | 0.0794147 | sugar transporter |
| CED82373 | −1.08484 | 0.0778048 | Microtubule-binding protein involved in cell cycle control |
| CED82356 | −1.08878 | 0.1232547 | GDP-mannose pyrophosphorylase |
| CED83350 | −1.09326 | 0.1295739 | signal recognition particle protein |
| CED83064 | −1.09776 | 0.0126954 | glutamate-5-semialdehyde dehydrogenase |
| CED83338 | −1.11182 | 0.1715042 | protein phosphatase |
| CED85589 | −1.11338 | 0.1017034 | Predicted proline-serine-threonine phosphatase- interacting protein (PSTPIP) |
| CED83853 | −1.11353 | 0.0735099 | dead-domain-containing protein |
| CDZ98315 | −1.11958 | 0.0490554 | Ribosomal protein S5 |
| CDZ96576 | −1.12389 | 0.008825 | sly1 vesicle trafficking sec1-like protein |
| CED83290 | −1.1313 | 0.0161918 | trehalose 6-phosphate phosphatase |
| CED82302 | −1.13258 | 0.1541232 | WD40 repeat-containing protein |
| CED84865 | −1.14211 | 0.1563611 | mitochondrial 50s ribosomal protein I3 |
| CED82772 | −1.14646 | 0.130641 | Histidine-specific methyltransferase, SAM- dependent |
| CED83357 | −1.14982 | 0.1568805 | 60s ribosomal protein I7 |
| CED84941 | −1.15648 | 0.0427497 | Predicted hydrolases or acyltransferases (alpha/beta hydrolase superfamily) |
| CED84219 | −1.15953 | 0.130641 | -trehalose-phosphate synthase (udp-forming) |
| CED84803 | −1.16039 | 0.0871441 | imp dehydrogenase |
| CED82043 | −1.17161 | 0.0643306 | Exosomal 3′-5′ exoribonuclease complex subunit Rrp40 |
| CDZ97030 | −1.17282 | 0.0026732 | succinate:fumarate antiporter |
| CDZ97070 | −1.1786 | 0.1706431 | glutathione peroxidase |
| CED84796 | −1.18436 | 0.0794704 | Delta 12 fatty acid desaturase |
| CED82849 | −1.19242 | 0.067386 | transaldolase |
| CED82649 | −1.19995 | 0.0993953 | phosphatase |
| CED84960 | −1.21113 | 0.0126954 | gtp binding protein 4 |
| CED82693 | −1.21672 | 0.1706431 | arm repeat-containing protein |
| CED83746 | −1.22831 | 0.1583372 | hypothetical protein |
| CDZ96477 | −1.23733 | 0.0037331 | ornithine carbamoyltransferase |
| CDZ96620 | −1.238 | 9.181E−05 | Fatty acid synthase complex subunit alpha |
| CED83014 | −1.24085 | 0.1715042 | nad-dependent formate dehydrogenase |
| CDZ96709 | −1.24506 | 0.0509018 | coatomer subunit gamma |
| CDZ97588 | −1.24644 | 0.0692198 | RP-S18, MRPS18, rpsR |
| CED83573 | −1.24929 | 0.0470041 | UDP-N-acetylglucosamine pyrophosphorylase |
| CDZ98425 | −1.25161 | 0.0147147 | aldehyde dehydrogenase |
| CDZ98564 | −1.25786 | 0.0289389 | pyruvate kinase |
| CED82578 | −1.26332 | 0.1079024 | hypothetical protein |
| CED84238 | −1.26644 | 0.0123918 | gtp-binding protein ypt3 |
| CED83765 | −1.27793 | 0.008825 | carbamoyl-phosphate synthase |
| CED84213 | −1.29049 | 0.0771834 | ras-domain-containing protein |
| CDZ98136 | −1.30498 | 0.1706431 | Vesicle trafficking protein Sec1 |
| CED82875 | −1.33021 | 0.0957369 | p-loop containing nucleoside triphosphate hydrolase protein |
| CDZ97947 | −1.34958 | 0.157889 | hypothetical protein |
| CED83361 | −1.35397 | 0.0139816 | dihydroxy-acid dehydratase |
| CDZ97373 | −1.36287 | 0.0751553 | Ribosome recycling factor domain |
| CDZ97245 | −1.37 | 0.0062306 | acetyl-hydrolase |
| CED84039 | −1.39997 | 0.0961632 | Glycosyl transferase, family 1 |
| CED84809 | −1.40948 | 0.0147147 | mago nashi |
| CDZ98813 | −1.41592 | 0.17568 | WD40/YVTN repeat-like-containing domain |
| CED83572 | −1.41975 | 0.0141757 | atp-citrate synthase |
| CED85129 | −1.44133 | 0.0554217 | isocitrate lyase |
| CED82387 | −1.44764 | 0.0294131 | GST, gst |
| CED82450 | −1.46422 | 0.0783197 | amidophosphoribosyltransferase |
| CDZ97923 | −1.47301 | 0.1097654 | d-arabinitol 2-dehydrogenase |
| CED84763 | −1.4734 | 0.0957766 | SPCS2, SPC2 |
| CDZ98236 | −1.47606 | 0.0499431 | Alcohol dehydrogenase, class V |
| CED84181 | −1.47713 | 0.0342256 | uridylate kinase |
| CED83904 | −1.48705 | 0.1150287 | swi snf complex protein |
| CED82339 | −1.52561 | 0.1709508 | Protein involved in Snf1 protein kinase complex assembly |
| CDZ97501 | −1.5258 | 0.087982 | p-loop containing nucleoside triphosphate hydrolase protein |
| CED85216 | −1.54086 | 0.0032648 | Aromatic amino acid aminotransferase and related proteins |
| CDZ96990 | −1.56918 | 0.0342256 | upf0041-domain-containing protein |
| CED83533 | −1.58921 | 0.0699152 | Protein of unknown function DUF3602 |
| CDZ97740 | −1.61523 | 0.0614175 | gtp-binding protein |
| CED83642 | −1.61929 | 0.1378419 | Uncharacterized conserved protein |
| CED83331 | −1.63302 | 0.0314857 | 60s ribosomal protein I11 |
| CED84705 | −1.63799 | 0.0270372 | arginine biosynthesis protein |
| CED82059 | −1.63961 | 0.0026732 | fk506-binding protein 2 |
| CED83630 | −1.64796 | 0.0037331 | glucose-6-phosphate isomerase |
| CDZ97129 | −1.67776 | 0.1289963 | Predicted regulator of rRNA gene transcription (MYB-binding protein) |
| CED83333 | −1.68454 | 0.0026273 | pyruvate decarboxylase |
| CED84473 | −1.68967 | 0.0457587 | Translational repressor Pumilio/PUF3 and related RNA-binding proteins (Puf superfamily) |
| CDZ97012 | −1.69367 | 0.0037331 | alcohol |
| CDZ98664 | −1.71814 | 0.0075757 | glycoside hydrolase family 13 protein |
| CED83236 | −1.73513 | 0.0026273 | epsilon dna polymerase |
| CDZ97011 | −1.73773 | 0.0050139 | 60s ribosomal protein I23 |
| CED83452 | −1.78054 | 0.0503176 | glycoside hydrolase family 13 protein |
| CED84972 | −1.79085 | 0.008062 | NADH-dehydrogenase (ubiquinone) |
| CDZ97924 | −1.81654 | 0.1672259 | hypothetical protein |
| CED84831 | −1.82825 | 0.1089294 | -like helicase 1 |
| CDZ97488 | −1.87594 | 0.0126954 | isocitrate dehydrogenase |
| CDZ98179 | −1.89763 | 0.0005055 | Acetyl CoA carboxylase |
| CED82102 | −1.90183 | 0.0274228 | tetrapyrrole biosynthesis porphobilinogen synthase |
| CED83672 | −1.91539 | 0.0751553 | hypothetical protein |
| CED84851 | −1.96978 | 0.0345283 | acetylornithine aminotransferase |
| CED84556 | −1.97107 | 0.0050139 | phosphoketolase |
| CDZ96541 | −1.97312 | 0.0993953 | atp phosphoribosyltransferase |
| CED84497 | −1.97839 | 0.0993953 | modular protein with glycoside hydrolase family 13 and glycosyltransferase family 5 domains |
| CED85401 | −2.09905 | 0.0122113 | |
| CDZ96394 | −2.10898 | 0.0503176 | phosphatidylethanolamine n-methyltransferase |
| CED83656 | −2.11939 | 0.1273922 | Delta 9 fatty acid desaturase |
| CED82203 | −2.24415 | 0.0289389 | metal resistance protein ycf1 |
| CED83962 | −2.41422 | 0.0666375 | glycoside hydrolase family 3 protein |
| CDZ96382 | −2.78513 | 0.008825 | glycoside hydrolase family 32 protein |
| CED82966 | −3.24281 | 0.0345283 | Taurine catabolismdioxygenase TauD/TfdA |
| CDZ96436 | −3.52954 | 0.0032648 | alcohol |
| CED83529 | −4.59152 | 6.361E−05 | Maltase glucoamylase and related hydrolases, glycosyl hydrolase family 31 |

TABLE 20

| CDS | SEQ | | |
|---|---|---|---|
| Accession | ID NO | Table(s) | Variation(s) |
| CDZ98521 | 14894 | Table 14 and Table 15 | Z₁; Z₂₁; V₁ |
| CED83134 | 16402 | Table 14 only | Z₂; Z₁₄ |
| CED80058 | 13437 | Table 14 and Table 15 | Z₃; Z₂₂; V₅; V₆; V₇ |
| CED80056 | 13605 | Table 14 and Table 15 | Z₄; V₁₀; V₁₁ |
| CDZ96150 | 15263 | Table 14 and Table 15 | Z₅; V₁₅; V₁₆; V₁₇; V₁₈ |
| CDZ96151 | 15929 | Table 14 and Table 15 | Z₆; Z₇; V₂₁; V₂₂; V₂₃; |
| | | | V₂₄; V₂₅ |
| CDZ96152 | 17082 | Table 14 and Table 15 | Z₈; V₁₂ |
| CDZ96153 | 12953 | Table 14 only | Z₉; Z₁₀ |
| CED80060 | 13445 | Table 14 only | Z₁₁ |
| CED80061 | 13989 | Table 14 and Table 15 | Z₁₂; Z₁₃; V₁₃; V₁₄; V₂₆ |
| CED83656 | 17215 | Table 14 only | Z₁₅ |
| CED83814 | 17373 | Table 14 only | Z₁₆ |
| CDZ96789 | 18649 | Table 14 only | Z₁₇ |
| CDZ97186 | 19046 | Table 14 and Table 15 | Z₁₈; V₂ |
| CDZ98193 | 13853 | Table 14 and Table 15 | Z₁₉; V₃ |
| CDZ98468 | 14841 | Table 14 only | Z₂₀ |
| CDZ96332 | 15265 | Table 14 and Table 15 | Z₂₃; V₄ |
| CED83975 | 17534 | Table 14 only | Z₂₄ |
| CDZ98804 | 15177 | Table 14 only | Z₂₅ |
| CDZ96154 | 13440 | Table 15 only | V₈; V₉ |
| CDZ96333 | 15928 | Table 15 only | V₁₉; V₂₀ |

1. An isolated nucleic acid comprising a variant of a nucleotide sequence set forth in:
SEQ ID NO: 14894 (corresponding to CDS accession CDZ98521 of X. dendrorhous CBS 6938);
SEQ ID NO: 16402 (corresponding to CDS accession CED83134 of X. dendrorhous CBS 6938);
SEQ ID NO: 13437 (corresponding to CDS accession CED80058 of X. dendrorhous CBS 6938);
SEQ ID NO: 13605 (corresponding to CDS accession CED80056 of X. dendrorhous CBS 6938);
SEQ ID NO: 15263 (corresponding to CDS accession CDZ96150 of X. dendrorhous CBS 6938);
SEQ ID NO: 15929 (corresponding to CDS accession CDZ96151 of X. dendrorhous CBS 6938);
SEQ ID NO: 17082 (corresponding to CDS accession CDZ96152 of X. dendrorhous CBS 6938);
SEQ ID NO: 12953 (corresponding to CDS accession CDZ96153 of X. dendrorhous CBS 6938);
SEQ ID NO: 13445 (corresponding to CDS accession CED80060 of X. dendrorhous CBS 6938);
SEQ ID NO: 13989 (corresponding to CDS accession CED80061 of X. dendrorhous CBS 6938);
SEQ ID NO: 17215 (corresponding to CDS accession CED83656 of X. dendrorhous CBS 6938);
SEQ ID NO:17373 (corresponding to CDS accession CED83814 of X. dendrorhous CBS 6938);
SEQ ID NO: 18649 (corresponding to CDS accession CDZ96789 of X. dendrorhous CBS 6938);
SEQ ID NO: 19046 (corresponding to CDS accession CDZ97186 of X. dendrorhous CBS 6938);
SEQ ID NO: 13853 (corresponding to CDS accession CDZ98193 of X. dendrorhous CBS 6938);
SEQ ID NO: 14841 (corresponding to CDS accession CDZ98468 of X. dendrorhous CBS 6938);
SEQ ID NO: 15265 (corresponding to CDS accession CDZ96332 of X. dendrorhous CBS 6938);
SEQ ID NO: 17534 (corresponding to CDS accession CED83975 of X. dendrorhous CBS 6938);
SEQ ID NO: 15177 (corresponding to CDS accession CDZ98804 of X. dendrorhous CBS 6938);
SEQ ID NO: 13440 (corresponding to CDS accession CDZ96154 of X. dendrorhous CBS 6938); or
SEQ ID NO:15928 (corresponding to CDS accession CDZ96333 of X. dendrorhous CBS 6938),
wherein the variant is at least 80% identical to the nucleotide sequence, or a fragment of the isolated nucleic acid.

2-23. (canceled)

24. An isolated protein encoded by the isolated nucleic acid of claim 1, or a fragment or derivative thereof.

25. The isolated protein of claim 24, wherein the protein is of a protein classification selected from ferredoxin/adrenodoxin reductase; cytochrome; ATP synthase; NADH dehydrogenase; fatty acid desaturase; Acyl-CoA-oxidase; pantothenate kinase; polyphosphate multikinase; G protein-coupled receptor; and succinate dehydrogenase.

26. The isolated protein of claim 24, wherein the protein is selected from ferredoxin/adrenodoxin reductase; mitochondrial cytochrome b2; cytochrome b; cytochrome c oxidase subunit 1; ATP synthase subunit 6; NADH dehydrogenase subunit 4; cytochrome c oxidase subunit 2; cytochrome c oxidase subunit 3; NADH dehydrogenase subunit 2; NADH dehydrogenase subunit 5; NADH dehydrogenase subunit 6; cytochrome c oxidase subunit 3; delta 9 fatty acid desaturase; Acyl-CoA-oxidase; pantothenate kinase PanK; geranylgeranyl pyrophosphate synthase; fumarate reductase; sucrose transporter; inositol polyphosphate multikinase, ARGR transcription regulatory complex component; G protein-coupled receptor, rhodopsin-like; succinate dehydrogenase; and ATP synthase subunit mitochondrial.

27. A method of modifying a nucleic acid including a step of changing one or more nucleotides of the nucleic acid to produce the isolated nucleic acid of any one of claim 1.

28. (canceled)

29. A genetic vector or construct comprising the isolated nucleic acid of any one of claim 1.

30. (canceled)

31. The vector or construct of claim 29, wherein the vector or construct is adapted for protein expression in yeast.

32. (canceled)

33. The vector or construct of claim 29, wherein the vector or construct is adapted for gene silencing in yeast.

34. (canceled)

35. The vector or construct of claim 29, wherein the vector or construct is adapted for genome editing in yeast.

36. An isolated cell comprising the nucleic acid of claim 1.

37-42. (canceled)

43. The isolated cell of claim 36, wherein the cell is a yeast cell.

44. The isolated cell of claim 43, wherein the cell is a Xanthophyllomyces cell or a Xanthophyllomyces dendrorhous cell.

45. An isolated yeast strain comprising the cell claim 43.

46-47. (canceled)

48. A method of producing astaxanthin including a step of expressing the isolated nucleic acid of claim 1 in vitro or in vivo, to thereby produce the astaxanthin.

49. A method of producing astaxanthin including a step of performing metabolism with the isolated cell of claim 36, to thereby produce the astaxanthin.

50. The method of claim 49, wherein the step of performing metabolism is or includes a step of performing fermentation with the isolated cell.

51. The method of claim 50, including a step of combining the cell or organism with a nitrogen source metabolite, such as malt extract.

52. (canceled)

53. The method of claim 50, including a step of combining the cell with a carbon source metabolite, such as molasses.

54-62. (canceled)

63. The isolated nucleic acid of claim 1, wherein the variant nucleotide sequence affects amino acid sequence or expression of an encoded protein or protein fragment.

64. An isolated cell, wherein the isolated cell has been mutagenised or genetically modified to alter expression of a protein encoded by:
SEQ ID NO: 14894 (corresponding to CDS accession CDZ98521 of X. dendrorhous CBS 6938);
SEQ ID NO: 16402 (corresponding to CDS accession CED83134 of X. dendrorhous CBS 6938);
SEQ ID NO: 13437 (corresponding to CDS accession CED80058 of X. dendrorhous CBS 6938);
SEQ ID NO: 13605 (corresponding to CDS accession CED80056 of X. dendrorhous CBS 6938);
SEQ ID NO: 15263 (corresponding to CDS accession CDZ96150 of X. dendrorhous CBS 6938);
SEQ ID NO: 15929 (corresponding to CDS accession CDZ96151 of X. dendrorhous CBS 6938);
SEQ ID NO: 17082 (corresponding to CDS accession CDZ96152 of X. dendrorhous CBS 6938);
SEQ ID NO: 12953 (corresponding to CDS accession CDZ96153 of X. dendrorhous CBS 6938);
SEQ ID NO: 13445 (corresponding to CDS accession CED80060 of X. dendrorhous CBS 6938);
SEQ ID NO: 13989 (corresponding to CDS accession CED80061 of X. dendrorhous CBS 6938);
SEQ ID NO: 17215 (corresponding to CDS accession CED83656 of X. dendrorhous CBS 6938);
SEQ ID NO:17373 (corresponding to CDS accession CED83814 of X. dendrorhous CBS 6938);
SEQ ID NO: 18649 (corresponding to CDS accession CDZ96789 of X. dendrorhous CBS 6938);
SEQ ID NO: 19046 (corresponding to CDS accession CDZ97186 of X. dendrorhous CBS 6938);
SEQ ID NO: 13853 (corresponding to CDS accession CDZ98193 of X. dendrorhous CBS 6938);
SEQ ID NO: 14841 (corresponding to CDS accession CDZ98468 of X. dendrorhous CBS 6938);
SEQ ID NO: 15265 (corresponding to CDS accession CDZ96332 of X. dendrorhous CBS 6938);
SEQ ID NO: 17534 (corresponding to CDS accession CED83975 of X. dendrorhous CBS 6938);
SEQ ID NO: 15177 (corresponding to CDS accession CDZ98804 of X. dendrorhous CBS 6938);
SEQ ID NO: 13440 (corresponding to CDS accession CDZ96154 of X. dendrorhous CBS 6938); or
SEQ ID NO:15928 (corresponding to CDS accession CDZ96333 of X. dendrorhous CBS 6938),
or to alter expression of a protein fragment or derivative of the protein.
